SEQUENCE LISTING (300 sequences)

SEQ ID NO: 1 - DNA, 1155 bp, Chlamydomonas reinhardtii
atgcaggtgtatgggtacgaggtcgtgggctgggaggaggcgcacgcgaaggagcccaagctcccggcggcggacccatacgcccctagccagctggtgacacccttggactcacagcagcagcaacagcagcagcaacagccgccgccgccatctgcggcctccaaggcttcgccactgggcgtgcccagacacggccagcgaaccatcttcaatgtggaggtgcggcgtccgagcagtttcgcgtcggcagccgaacagcagcagcaccagttggcggttctgcgtgctgattgcgagctcgtgattatacagcgcgcggaggcggcgcagggcccgccagcccccgaggagcatacgtcggctggggcggcggcggccaggggcccagcagcaggcggagctgaagcggcggaggcggccgcgccggtgccgtgcgatgaggtggtgaccctggtgccggccttcttcttctgctgcagtagcggcggccgcgtgacggtgcggctgcggccggggcgggatggctacgtggcaggcgaggcggcggaggtggtggtcgaggttgacaaccggtcgaatcaggagtttcgggatgtgcggcttgaagtggagcgccgcctcacattggtcagcaacagcgccggcggaggcggtagcgccggcagcagcggcagcggcagtagcagcgccaccgcggggcttgtgccgggatgcttcactgaagaggagcggatcttcaagagcaagaccacggcctgctacctgggagccaacgcgctgcggctgccggtgcccctgccctccaacacgccgccctccacctccggcgcgcttgtgcgctgctcctacaccgccacggtggaggtgctgccggcgtcggcgacagcgctgcgcggcgcggcgccgccgcggctgcgtgtgccgctgaccgtgttcgcatccgcgccgagctcgttcgccacggcggcggcacggcatgctcacctgcagcaggacgcaagcgagcaagcgccggcgcacgtgttggtggtggtgccgcccgtggatgtagtgctccccgcagctgcgccgcagctgcctcccaccgccgaggtaaatgtcaaacagcacaacggcgtggctggcgcaaacccgatgtacgcgggcccgtag

SEQ ID NO: 2 - DNA, 1149 bp, Chlamydomonas reinhardtii
caggtgtatgggtacgaggtcgtgggctgggaggaggcgcacgcgaaggagcccaagctcccggcggcggacccatacgcccctagccagctggtgacacccttggactcacagcagcagcaacagcagcagcaacagccgccgccgccatctgcggcctccaaggcttcgccactgggcgtgcccagacacggccagcgaaccatcttcaatgtggaggtgcggcgtccgagcagtttcgcgtcggcagccgaacagcagcagcaccagttggcggttctgcgtgctgattgcgagctcgtgattatacagcgcgcggaggcggcgcagggcccgccagcccccgaggagcatacgtcggctggggcggcggcggccaggggcccagcagcaggcggagctgaagcggcggaggcggccgcgccggtgccgtgcgatgaggtggtgaccctggtgccggccttcttcttctgctgcagtagcggcggccgcgtgacggtgcggctgcggccggggcgggatggctacgtggcaggcgaggcggcggaggtggtggtcgaggttgacaaccggtcgaatcaggagtttcgggatgtgcggcttgaagtggagcgccgcctcacattggtcagcaacagcgccggcggaggcggtagcgccggcagcagcggcagcggcagtagcagcgccaccgcggggcttgtgccgggatgcttcactgaagaggagcggatcttcaagagcaagaccacggcctgctacctgggagccaacgcgctgcggctgccggtgcccctgccctccaacacgccgccctccacctccggcgcgcttgtgcgctgctcctacaccgccacggtggaggtgctgccggcgtcggcgacagcgctgcgcggcgcggcgccgccgcggctgcgtgtgccgctgaccgtgttcgcatccgcgccgagctcgttcgccacggcggcggcacggcatgctcacctgcagcaggacgcaagcgagcaagcgccggcgcacgtgttggtggtggtgccgcccgtggatgtagtgctccccgcagctgcgccgcagctgcctcccaccgccgaggtaaatgtcaaacagcacaacggcgtggctggcgcaaacccgatgtacgcgggcccg

SEQ ID NO: 3 - DNA, 1152 bp, Artificial Sequence
  codon optimized
atgcaagtgtatggttacgaggtggtgggttgggaggaggctcatgctaaggagcccaagctgcccgcggccgacccctacgccccatcccaactggtcactccgctggacagccagcagcagcaacagcagcaacaacaaccgccgcccccgtccgccgccagcaaggcctccccgctcggcgtgcctcgtcacggtcaacgcacaattttcaacgtcgaggtccggcgtccctcgtccttcgcgtcggcggcagagcaacaacagcaccagctggccgtgctgcgggcggactgcgagctcgtcatcatccagcgcgcggaggccgcccagggcccaccagcccccgaggagcatacgtccgccggtgccgctgccgctcgcgggccagcggctgggggtgctgaggcggcggaggcggctgcccccgtgccgtgcgacgaggtggtgacgctggtccccgccttctttttctgctgctcgtccgggggtcgcgtgaccgtgcgcctgcgcccaggccgcgacggttacgtggctggcgaggccgctgaggtcgtggtggaggtggacaaccggagcaaccaggagttccgtgacgtgcgcctggaggtcgagcgccgcctcacgctggtgtcgaactcggcgggtggcggcggctcggcggggtcctcgggctcgggcagctcgtccgctacggccggtctggtgccaggctgcttcacggaggaggagcggatcttcaagtcgaagacgacagcgtgttacctgggcgcgaacgccctgcgcctgccggtccccctgcccagcaacaccccgccttccacctcgggcgcgctggtgcgttgcagctataccgcgaccgtcgaggtgctgccggcgagcgcgacggcgctgcgtggggccgctcccccgcgtctccgtgtgccgctgaccgtgttcgcgtccgcgccttcgtcgttcgccaccgccgcagcccgccacgcgcacctgcaacaggacgccagcgagcaggcaccggcccacgtcctggtggtggtgccgcccgtggacgtggtgctgccagccgccgcaccccagctgcctcccaccgcggaggtgaacgtgaagcagcacaacggcgtggcgggcgccaaccccatgtacgccggtccc

SEQ ID NO: 4 - DNA, 1149 bp, Artificial Sequence
  codon optimized
caagtgtatggttacgaggtggtgggttgggaggaggctcatgctaaggagcccaagctgcccgcggccgacccctacgccccatcccaactggtcactccgctggacagccagcagcagcaacagcagcaacaacaaccgccgcccccgtccgccgccagcaaggcctccccgctcggcgtgcctcgtcacggtcaacgcacaattttcaacgtcgaggtccggcgtccctcgtccttcgcgtcggcggcagagcaacaacagcaccagctggccgtgctgcgggcggactgcgagctcgtcatcatccagcgcgcggaggccgcccagggcccaccagcccccgaggagcatacgtccgccggtgccgctgccgctcgcgggccagcggctgggggtgctgaggcggcggaggcggctgcccccgtgccgtgcgacgaggtggtgacgctggtccccgccttctttttctgctgctcgtccgggggtcgcgtgaccgtgcgcctgcgcccaggccgcgacggttacgtggctggcgaggccgctgaggtcgtggtggaggtggacaaccggagcaaccaggagttccgtgacgtgcgcctggaggtcgagcgccgcctcacgctggtgtcgaactcggcgggtggcggcggctcggcggggtcctcgggctcgggcagctcgtccgctacggccggtctggtgccaggctgcttcacggaggaggagcggatcttcaagtcgaagacgacagcgtgttacctgggcgcgaacgccctgcgcctgccggtccccctgcccagcaacaccccgccttccacctcgggcgcgctggtgcgttgcagctataccgcgaccgtcgaggtgctgccggcgagcgcgacggcgctgcgtggggccgctcccccgcgtctccgtgtgccgctgaccgtgttcgcgtccgcgccttcgtcgttcgccaccgccgcagcccgccacgcgcacctgcaacaggacgccagcgagcaggcaccggcccacgtcctggtggtggtgccgcccgtggacgtggtgctgccagccgccgcaccccagctgcctcccaccgcggaggtgaacgtgaagcagcacaacggcgtggcgggcgccaaccccatgtacgccggtccc

SEQ ID NO: 5 - DNA, 1212 bp, Artificial Sequence
  codon optimized
  misc_feature (1153)..(1158): AgeI restriction site
  misc_feature (1159)..(1182): FLAG Tag
  misc_feature (1183)..(1203): MAT Tag
  misc_feature (1204)..(1209): AgeI restriction site
atgcaagtgtatggttacgaggtggtgggttgggaggaggctcatgctaaggagcccaagctgcccgcggccgacccctacgccccatcccaactggtcactccgctggacagccagcagcagcaacagcagcaacaacaaccgccgcccccgtccgccgccagcaaggcctccccgctcggcgtgcctcgtcacggtcaacgcacaattttcaacgtcgaggtccggcgtccctcgtccttcgcgtcggcggcagagcaacaacagcaccagctggccgtgctgcgggcggactgcgagctcgtcatcatccagcgcgcggaggccgcccagggcccaccagcccccgaggagcatacgtccgccggtgccgctgccgctcgcgggccagcggctgggggtgctgaggcggcggaggcggctgcccccgtgccgtgcgacgaggtggtgacgctggtccccgccttctttttctgctgctcgtccgggggtcgcgtgaccgtgcgcctgcgcccaggccgcgacggttacgtggctggcgaggccgctgaggtcgtggtggaggtggacaaccggagcaaccaggagttccgtgacgtgcgcctggaggtcgagcgccgcctcacgctggtgtcgaactcggcgggtggcggcggctcggcggggtcctcgggctcgggcagctcgtccgctacggccggtctggtgccaggctgcttcacggaggaggagcggatcttcaagtcgaagacgacagcgtgttacctgggcgcgaacgccctgcgcctgccggtccccctgcccagcaacaccccgccttccacctcgggcgcgctggtgcgttgcagctataccgcgaccgtcgaggtgctgccggcgagcgcgacggcgctgcgtggggccgctcccccgcgtctccgtgtgccgctgaccgtgttcgcgtccgcgccttcgtcgttcgccaccgccgcagcccgccacgcgcacctgcaacaggacgccagcgagcaggcaccggcccacgtcctggtggtggtgccgcccgtggacgtggtgctgccagccgccgcaccccagctgcctcccaccgcggaggtgaacgtgaagcagcacaacggcgtggcgggcgccaaccccatgtacgccggtcccaccggtgactacaaggacgacgacgacaagcacaaccaccgccataagcacaccggttga

SEQ ID NO: 6 - protein, 384 aa, Chlamydomonas reinhardtii
MQVYGYEVVGWEEAHAKEPKLPAADPYAPSQLVTPLDSQQQQQQQQQPPPPSAASKASPLGVPRHGQRTIFNVEVRRPSSFASAAEQQQHQLAVLRADCELVIIQRAEAAQGPPAPEEHTSAGAAAARGPAAGGAEAAEAAAPVPCDEVVTLVPAFFFCCSSGGRVTVRLRPGRDGYVAGEAAEVVVEVDNRSNQEFRDVRLEVERRLTLVSNSAGGGGSAGSSGSGSSSATAGLVPGCFTEEERIFKSKTTACYLGANALRLPVPLPSNTPPSTSGALVRCSYTATVEVLPASATALRGAAPPRLRVPLTVFASAPSSFATAAARHAHLQQDASEQAPAHVLVVVPPVDVVLPAAAPQLPPTAEVNVKQHNGVAGANPMYAGP

SEQ ID NO: 7 - protein, 403 aa, Artificial Sequence
  modified protein sequence
  MISC_FEATURE (384)..(385): AgeI restriction site
  MISC_FEATURE (386)..(394): FLAG Tag
  MISC_FEATURE (395)..(401): MAT Tag
  MISC_FEATURE (402)..(403): AgeI restriction site
MQVYGYEVVGWEEAHAKEPKLPAADPYAPSQLVTPLDSQQQQQQQQQPPPPSAASKASPLGVPRHGQRTIFNVEVRRPSSFASAAEQQQHQLAVLRADCELVIIQRAEAAQGPPAPEEHTSAGAAAARGPAAGGAEAAEAAAPVPCDEVVTLVPAFFFCCSSGGRVTVRLRPGRDGYVAGEAAEVVVEVDNRSNQEFRDVRLEVERRLTLVSNSAGGGGSAGSSGSGSSSATAGLVPGCFTEEERIFKSKTTACYLGANALRLPVPLPSNTPPSTSGALVRCSYTATVEVLPASATALRGAAPPRLRVPLTVFASAPSSFATAAARHAHLQQDASEQAPAHVLVVVPPVDVVLPAAAPQLPPTAEVNVKQHNGVAGANPMYAGPTGDYKDDDDKHNHRHKHTG

SEQ ID NO: 8 - DNA, 1356 bp, Chlamydomonas reinhardtii
atgcaggtgtatgggtacgaggtcgtgggctgggaggaggcgcacgcgaaggagcccaagctcccggcggcggacccatacgcccctagccagctggtgacacccttggactcacagcagcagcaacagcagcagcaacagccgccgccgccatctgcggcctccaaggcttcgccactgggcgtgcccagacacggccagcgaaccatcttcaatgtatgcgtcccactgctggcgggcgggcggcaagtgctgccgccggggacgtacaggcttcccttccggctgcaactccctgcagatctgccagggacgtttcggctggccggcacaccagcacgcaccattggagacgtgagctaccggaacctctctggcgaggtcagctacggcttgcaggtggaggtgcggcgtccgagcagtttcgcgtcggcagccgaacagcagcagcaccagttggcggttctgcgtgctgattgcgagctcgtgattatacagcgcgcggaggcggcgcagggcccgccagcccccgaggagcatacgtcggctggggcggcggcggccaggggcccagcagcaggcggagctgaagcggcggaggcggccgcgccggtgccgtgcgatgaggtggtgaccctggtgccggccttcttcttctgctgcagtagcggcggccgcgtgacggtgcggctgcggccggggcgggatggctacgtggcaggcgaggcggcggaggtggtggtcgaggttgacaaccggtcgaatcaggagtttcgggatgtgcggcttgaagtggagcgccgcctcacattggtcagcaacagcgccggcggaggcggtagcgccggcagcagcggcagcggcagtagcagcgccaccgcggggcttgtgccgggatgcttcactgaagaggagcggatcttcaagagcaagaccacggccgccctactaccgggagcctgctacctgggagccaacgcgctgcggctgccggtgcccctgccctccaacacgccgccctccacctccggcgcgcttgtgcgctgctcctacaccgccacggtggaggtgctgccggcgtcggcgacagcgctgcgcggcgcggcgccgccgcggctgcgtgtgccgctgaccgtgttcgcatccgcgccgagctcgttcgccacggcggcggcacggcatgctcacctgcagcaggacgcaagcgagcaagcgccggcgcacgtgttggtggtggtgccgcccgtggatgtagtgctccccgcagctgcgccgcagctgcctcccaccgccgaggtaaatgtcaaacagcacaacggcgtggctggcgcaaacccgatgtacgcgggcccgtag

SEQ ID NO: 9 - DNA, 1350 bp, Chlamydomonas reinhardtii
caggtgtatgggtacgaggtcgtgggctgggaggaggcgcacgcgaaggagcccaagctcccggcggcggacccatacgcccctagccagctggtgacacccttggactcacagcagcagcaacagcagcagcaacagccgccgccgccatctgcggcctccaaggcttcgccactgggcgtgcccagacacggccagcgaaccatcttcaatgtatgcgtcccactgctggcgggcgggcggcaagtgctgccgccggggacgtacaggcttcccttccggctgcaactccctgcagatctgccagggacgtttcggctggccggcacaccagcacgcaccattggagacgtgagctaccggaacctctctggcgaggtcagctacggcttgcaggtggaggtgcggcgtccgagcagtttcgcgtcggcagccgaacagcagcagcaccagttggcggttctgcgtgctgattgcgagctcgtgattatacagcgcgcggaggcggcgcagggcccgccagcccccgaggagcatacgtcggctggggcggcggcggccaggggcccagcagcaggcggagctgaagcggcggaggcggccgcgccggtgccgtgcgatgaggtggtgaccctggtgccggccttcttcttctgctgcagtagcggcggccgcgtgacggtgcggctgcggccggggcgggatggctacgtggcaggcgaggcggcggaggtggtggtcgaggttgacaaccggtcgaatcaggagtttcgggatgtgcggcttgaagtggagcgccgcctcacattggtcagcaacagcgccggcggaggcggtagcgccggcagcagcggcagcggcagtagcagcgccaccgcggggcttgtgccgggatgcttcactgaagaggagcggatcttcaagagcaagaccacggccgccctactaccgggagcctgctacctgggagccaacgcgctgcggctgccggtgcccctgccctccaacacgccgccctccacctccggcgcgcttgtgcgctgctcctacaccgccacggtggaggtgctgccggcgtcggcgacagcgctgcgcggcgcggcgccgccgcggctgcgtgtgccgctgaccgtgttcgcatccgcgccgagctcgttcgccacggcggcggcacggcatgctcacctgcagcaggacgcaagcgagcaagcgccggcgcacgtgttggtggtggtgccgcccgtggatgtagtgctccccgcagctgcgccgcagctgcctcccaccgccgaggtaaatgtcaaacagcacaacggcgtggctggcgcaaacccgatgtacgcgggcccg

SEQ ID NO: 10 - DNA, 1416 bp, Artificial Sequence
  modified nucleotide sequence
  misc_feature (1)..(6): XhoI restriction site
  misc_feature (1357)..(1362): AgeI restriction site
  misc_feature (1363)..(1386): FLAG Tag
  misc_feature (1387)..(1407): MAT Tag
  misc_feature (1408)..(1413): XmaI/AgeI restriction site
ctcgagcaggtgtatgggtacgaggtcgtgggctgggaggaggcgcacgcgaaggagcccaagctcccggcggcggacccatacgcccctagccagctggtgacacccttggactcacagcagcagcaacagcagcagcaacagccgccgccgccatctgcggcctccaaggcttcgccactgggcgtgcccagacacggccagcgaaccatcttcaatgtatgcgtcccactgctggcgggcgggcggcaagtgctgccgccggggacgtacaggcttcccttccggctgcaactccctgcagatctgccagggacgtttcggctggccggcacaccagcacgcaccattggagacgtgagctaccggaacctctctggcgaggtcagctacggcttgcaggtggaggtgcggcgtccgagcagtttcgcgtcggcagccgaacagcagcagcaccagttggcggttctgcgtgctgattgcgagctcgtgattatacagcgcgcggaggcggcgcagggcccgccagcccccgaggagcatacgtcggctggggcggcggcggccaggggcccagcagcaggcggagctgaagcggcggaggcggccgcgccggtgccgtgcgatgaggtggtgaccctggtgccggccttcttcttctgctgcagtagcggcggccgcgtgacggtgcggctgcggccggggcgggatggctacgtggcaggcgaggcggcggaggtggtggtcgaggttgacaaccggtcgaatcaggagtttcgggatgtgcggcttgaagtggagcgccgcctcacattggtcagcaacagcgccggcggaggcggtagcgccggcagcagcggcagcggcagtagcagcgccaccgcggggcttgtgccgggatgcttcactgaagaggagcggatcttcaagagcaagaccacggccgccctactaccgggagcctgctacctgggagccaacgcgctgcggctgccggtgcccctgccctccaacacgccgccctccacctccggcgcgcttgtgcgctgctcctacaccgccacggtggaggtgctgccggcgtcggcgacagcgctgcgcggcgcggcgccgccgcggctgcgtgtgccgctgaccgtgttcgcatccgcgccgagctcgttcgccacggcggcggcacggcatgctcacctgcagcaggacgcaagcgagcaagcgccggcgcacgtgttggtggtggtgccgcccgtggatgtagtgctccccgcagctgcgccgcagctgcctcccaccgccgaggtaaatgtcaaacagcacaacggcgtggctggcgcaaacccgatgtacgcgggcccgaccggtgactacaaggacgacgacgacaagcacaaccaccgccacaagcaccccggttaa

SEQ ID NO: 11 - DNA, 1356 bp, Artificial Sequence
  codon optimized
atgcaagtgtatggttacgaggtggtgggttgggaggaggctcatgctaaggagcccaagctgcccgcggccgacccctacgccccatcccaactggtcactccgctggacagccagcagcagcaacagcagcaacaacaaccgccgcccccgtccgccgccagcaaggcctccccgctcggcgtgcctcgtcacggtcaacgcacaattttcaacgtctgtgtgccactcctggctgggggccgtcaagtgctccctcccggcacctaccgcctgcccttccgcctccagctgccggctgacctgccaggcacgtttcgcctggccggcaccccggcgcgcacgatcggcgacgtgtcctaccggaacctgtccggggaggtgagctacggcctccaggtggaggtccggcgtccctcgtccttcgcgtcggcggcagagcaacaacagcaccagctggccgtgctgcgggcggactgcgagctcgtcatcatccagcgcgcggaggccgcccagggcccaccagcccccgaggagcatacgtccgccggtgccgctgccgctcgcgggccagcggctgggggtgctgaggcggcggaggcggctgcccccgtgccgtgcgacgaggtggtgacgctggtccccgccttctttttctgctgctcgtccgggggtcgcgtgaccgtgcgcctgcgcccaggccgcgacggttacgtggctggcgaggccgctgaggtcgtggtggaggtggacaaccggagcaaccaggagttccgtgacgtgcgcctggaggtcgagcgccgcctcacgctggtgtcgaactcggcgggtggcggcggctcggcggggtcctcgggctcgggcagctcgtccgctacggccggtctggtgccaggctgcttcacggaggaggagcggatcttcaagtcgaagacgacagcggcgctgctgccaggggcctgttacctgggcgcgaacgccctgcgcctgccggtccccctgcccagcaacaccccgccttccacctcgggcgcgctggtgcgttgcagctataccgcgaccgtcgaggtgctgccggcgagcgcgacggcgctgcgtggggccgctcccccgcgtctccgtgtgccgctgaccgtgttcgcgtccgcgccttcgtcgttcgccaccgccgcagcccgccacgcgcacctgcaacaggacgccagcgagcaggcaccggcccacgtcctggtggtggtgccgcccgtggacgtggtgctgccagccgccgcaccccagctgcctcccaccgcggaggtgaacgtgaagcagcacaacggcgtggcgggcgccaaccccatgtacgccggtccctag

SEQ ID NO: 12 - DNA, 1350 bp, Artificial Sequence
  codon optimized
caagtgtatggttacgaggtggtgggttgggaggaggctcatgctaaggagcccaagctgcccgcggccgacccctacgccccatcccaactggtcactccgctggacagccagcagcagcaacagcagcaacaacaaccgccgcccccgtccgccgccagcaaggcctccccgctcggcgtgcctcgtcacggtcaacgcacaattttcaacgtctgtgtgccactcctggctgggggccgtcaagtgctccctcccggcacctaccgcctgcccttccgcctccagctgccggctgacctgccaggcacgtttcgcctggccggcaccccggcgcgcacgatcggcgacgtgtcctaccggaacctgtccggggaggtgagctacggcctccaggtggaggtccggcgtccctcgtccttcgcgtcggcggcagagcaacaacagcaccagctggccgtgctgcgggcggactgcgagctcgtcatcatccagcgcgcggaggccgcccagggcccaccagcccccgaggagcatacgtccgccggtgccgctgccgctcgcgggccagcggctgggggtgctgaggcggcggaggcggctgcccccgtgccgtgcgacgaggtggtgacgctggtccccgccttctttttctgctgctcgtccgggggtcgcgtgaccgtgcgcctgcgcccaggccgcgacggttacgtggctggcgaggccgctgaggtcgtggtggaggtggacaaccggagcaaccaggagttccgtgacgtgcgcctggaggtcgagcgccgcctcacgctggtgtcgaactcggcgggtggcggcggctcggcggggtcctcgggctcgggcagctcgtccgctacggccggtctggtgccaggctgcttcacggaggaggagcggatcttcaagtcgaagacgacagcggcgctgctgccaggggcctgttacctgggcgcgaacgccctgcgcctgccggtccccctgcccagcaacaccccgccttccacctcgggcgcgctggtgcgttgcagctataccgcgaccgtcgaggtgctgccggcgagcgcgacggcgctgcgtggggccgctcccccgcgtctccgtgtgccgctgaccgtgttcgcgtccgcgccttcgtcgttcgccaccgccgcagcccgccacgcgcacctgcaacaggacgccagcgagcaggcaccggcccacgtcctggtggtggtgccgcccgtggacgtggtgctgccagccgccgcaccccagctgcctcccaccgcggaggtgaacgtgaagcagcacaacggcgtggcgggcgccaaccccatgtacgccggtccc

SEQ ID NO: 13 - DNA, 1416 bp, Artificial Sequence
  codon optimized
  misc_feature (1)..(6): XhoI restriction site
  misc_feature (1357)..(1362): AgeI restriction site
  misc_feature (1363)..(1386): FLAG Tag
  misc_feature (1387)..(1407): MAT Tag
  misc_feature (1408)..(1413): XmaI/AgeI restriction site
ctcgagcaagtgtatggttacgaggtggtgggttgggaggaggctcatgctaaggagcccaagctgcccgcggccgacccctacgccccatcccaactggtcactccgctggacagccagcagcagcaacagcagcaacaacaaccgccgcccccgtccgccgccagcaaggcctccccgctcggcgtgcctcgtcacggtcaacgcacaattttcaacgtctgtgtgccactcctggctgggggccgtcaagtgctccctcccggcacctaccgcctgcccttccgcctccagctgccggctgacctgccaggcacgtttcgcctggccggcaccccggcgcgcacgatcggcgacgtgtcctaccggaacctgtccggggaggtgagctacggcctccaggtggaggtccggcgtccctcgtccttcgcgtcggcggcagagcaacaacagcaccagctggccgtgctgcgggcggactgcgagctcgtcatcatccagcgcgcggaggccgcccagggcccaccagcccccgaggagcatacgtccgccggtgccgctgccgctcgcgggccagcggctgggggtgctgaggcggcggaggcggctgcccccgtgccgtgcgacgaggtggtgacgctggtccccgccttctttttctgctgctcgtccgggggtcgcgtgaccgtgcgcctgcgcccaggccgcgacggttacgtggctggcgaggccgctgaggtcgtggtggaggtggacaaccggagcaaccaggagttccgtgacgtgcgcctggaggtcgagcgccgcctcacgctggtgtcgaactcggcgggtggcggcggctcggcggggtcctcgggctcgggcagctcgtccgctacggccggtctggtgccaggctgcttcacggaggaggagcggatcttcaagtcgaagacgacagcggcgctgctgccaggggcctgttacctgggcgcgaacgccctgcgcctgccggtccccctgcccagcaacaccccgccttccacctcgggcgcgctggtgcgttgcagctataccgcgaccgtcgaggtgctgccggcgagcgcgacggcgctgcgtggggccgctcccccgcgtctccgtgtgccgctgaccgtgttcgcgtccgcgccttcgtcgttcgccaccgccgcagcccgccacgcgcacctgcaacaggacgccagcgagcaggcaccggcccacgtcctggtggtggtgccgcccgtggacgtggtgctgccagccgccgcaccccagctgcctcccaccgcggaggtgaacgtgaagcagcacaacggcgtggcgggcgccaaccccatgtacgccggtcccaccggtgactacaaggacgacgacgacaagcacaaccaccgccacaagcaccccggttaa

SEQ ID NO: 14 - protein, 451 aa, Chlamydomonas reinhardtii
MQVYGYEVVGWEEAHAKEPKLPAADPYAPSQLVTPLDSQQQQQQQQQPPPPSAASKASPLGVPRHGQRTIFNVCVPLLAGGRQVLPPGTYRLPFRLQLPADLPGTFRLAGTPARTIGDVSYRNLSGEVSYGLQVEVRRPSSFASAAEQQQHQLAVLRADCELVIIQRAEAAQGPPAPEEHTSAGAAAARGPAAGGAEAAEAAAPVPCDEVVTLVPAFFFCCSSGGRVTVRLRPGRDGYVAGEAAEVVVEVDNRSNQEFRDVRLEVERRLTLVSNSAGGGGSAGSSGSGSSSATAGLVPGCFTEEERIFKSKTTAALLPGACYLGANALRLPVPLPSNTPPSTSGALVRCSYTATVEVLPASATALRGAAPPRLRVPLTVFASAPSSFATAAARHAHLQQDASEQAPAHVLVVVPPVDVVLPAAAPQLPPTAEVNVKQHNGVAGANPMYAGP

SEQ ID NO: 15 - protein, 471 aa, Artificial Sequence
  modified protein sequence
  MISC_FEATURE (1)..(2): XhoI restriction site
  MISC_FEATURE (453)..(454): AgeI restriction site
  MISC_FEATURE (455)..(462): FLAG Tag
  MISC_FEATURE (463)..(469): MAT Tag
  MISC_FEATURE (470)..(471): XmaI/AgeI restriction site
LEQVYGYEVVGWEEAHAKEPKLPAADPYAPSQLVTPLDSQQQQQQQQQPPPPSAASKASPLGVPRHGQRTIFNVCVPLLAGGRQVLPPGTYRLPFRLQLPADLPGTFRLAGTPARTIGDVSYRNLSGEVSYGLQVEVRRPSSFASAAEQQQHQLAVLRADCELVIIQRAEAAQGPPAPEEHTSAGAAAARGPAAGGAEAAEAAAPVPCDEVVTLVPAFFFCCSSGGRVTVRLRPGRDGYVAGEAAEVVVEVDNRSNQEFRDVRLEVERRLTLVSNSAGGGGSAGSSGSGSSSATAGLVPGCFTEEERIFKSKTTAALLPGACYLGANALRLPVPLPSNTPPSTSGALVRCSYTATVEVLPASATALRGAAPPRLRVPLTVFASAPSSFATAAARHAHLQQDASEQAPAHVLVVVPPVDVVLPAAAPQLPPTAEVNVKQHNGVAGANPMYAGPTGDYKDDDDKHNHRHKHPG

SEQ ID NO: 16 - DNA, 1215 bp, Artificial Sequence
  codon optimized
  misc_feature (1)..(6): XhoI restriction site
  misc_feature (1156)..(1161): AgeI restriction site
  misc_feature (1162)..(1185): FLAG Tag
  misc_feature (1186)..(1206): MAT Tag
  misc_feature (1207)..(1212): AgeI restriction site
ctcgagcaagtgtatggttacgaggtggtgggttgggaggaggctcatgctaaggagcccaagctgcccgcggccgacccctacgccccatcccaactggtcactccgctggacagccagcagcagcaacagcagcaacaacaaccgccgcccccgtccgccgccagcaaggcctccccgctcggcgtgcctcgtcacggtcaacgcacaattttcaacgtcgaggtccggcgtccctcgtccttcgcgtcggcggcagagcaacaacagcaccagctggccgtgctgcgggcggactgcgagctcgtcatcatccagcgcgcggaggccgcccagggcccaccagcccccgaggagcatacgtccgccggtgccgctgccgctcgcgggccagcggctgggggtgctgaggcggcggaggcggctgcccccgtgccgtgcgacgaggtggtgacgctggtccccgccttctttttctgctgctcgtccgggggtcgcgtgaccgtgcgcctgcgcccaggccgcgacggttacgtggctggcgaggccgctgaggtcgtggtggaggtggacaaccggagcaaccaggagttccgtgacgtgcgcctggaggtcgagcgccgcctcacgctggtgtcgaactcggcgggtggcggcggctcggcggggtcctcgggctcgggcagctcgtccgctacggccggtctggtgccaggctgcttcacggaggaggagcggatcttcaagtcgaagacgacagcgtgttacctgggcgcgaacgccctgcgcctgccggtccccctgcccagcaacaccccgccttccacctcgggcgcgctggtgcgttgcagctataccgcgaccgtcgaggtgctgccggcgagcgcgacggcgctgcgtggggccgctcccccgcgtctccgtgtgccgctgaccgtgttcgcgtccgcgccttcgtcgttcgccaccgccgcagcccgcaccgcgaccctgcaacaggacgccagcgagcaggcaccggcccacgtcctggtggtggtgccgcccgtggacgtggtgctgccagccgccgcaccccagctgcctcccaccgcggaggtgaacgtgaagcagcacaacggcgtggcgggcgccaaccccatgtacgccggtcccaccggtgactacaaggacgacgacgacaagcacaaccaccgccataagcacaccggttga

SEQ ID NO: 17 - DNA, 1215 bp, Artificial Sequence
  codon optimized
  misc_feature (1)..(6): Xhol restriction site
  misc_feature (1156)..(1161): AgeI restriction site
  misc_feature (1162)..(1185): FLAG Tag
  misc_feature (1186)..(1206): MAT Tag
  misc_feature (1207)..(1212): AgeI restriction site
ctcgagcaagtgtatggttacgaggtggtgggttgggaggaggctcatgctaaggagcccaagctgcccgcggccgacccctacgccccatcccaactggtcactccgctggacagccagcagcagcaacagcagcaacaacaaccgccgcccccgtccgccgccagcaaggcctccccgctcggcgtgcctcgtcacggtcaacgcacaattttcaacgtcgaggtccggcgtccctcgtccttcgcgtcggcggcagagcaacaacagcaccagctggccgtgctgcgggcggactgcgagctcgtcatcatccagcgcgcggaggccgcccagggcccaccagcccccgaggagcatacgtccgccggtgccgctgccgctcgcgggccagcggctgggggtgctgaggcggcggaggcggctgcccccgtgccgtgcgacgaggtggtgacgctggtccccgccttctttttctgctgctcgtccgggggtcgcgtgaccgtgcgcctgcgcccaggccgcgacggttacgtggctggcgaggccgctgaggtcgtggtggaggtggacaaccggagcaaccaggagttccgtgacgtgcgcctggaggtcgagcgccgcctcacgctggtgtcgaactcggcgggtggcggcggctcggcggggtcctcgggctcgggcagctcgtccgctacggccggtctggtgccaggctgcttcacggaggaggagcggatcttcaagtcgaagacgacagcgtgttacctgggcgcgaacgccctgcgcctgccggtccccctgcccagcaacaccccgccttccacctcgggcgcgctggtgcgttgcagctataccgcgaccgtcgaggtgctgccggcgagcgcgacggcgctgcgtggggccgctcccccgcgtctccgtgtgccgctgaccgtgttcgcgtccgcgccttcgtcgttcgccaccgccgcagcccgccacgcgcacctgcaacaggacgccagcgagcaggcaccggccaccgtcctggtggtggtgccgcccgtggacgtggtgctgccagccgccgcaccccagctgcctcccaccgcggaggtgaacgtgaagcagcacaacggcgtggcgggcgccaaccccatgtacgccggtcccaccggtgactacaaggacgacgacgacaagcacaaccaccgccataagcacaccggttga

SEQ ID NO: 18 - DNA, 1215 bp, Artificial Sequence
  codon optimized
  misc_feature (1)..(6): XhoI restriction site
  misc_feature (1156)..(1161): AgeI restriction site
  misc_feature (1162)..(1185): FLAG Tag
  misc_feature (1186)..(1206): MAT Tag
  misc_feature (1207)..(1212): AgeI restriction site
ctcgagcaagtgtatggttacgaggtggtgggttgggaggaggctcatgctaaggagcccaagctgcccgcggccgacccctacgccccatcccaactggtcactccgctggacagccagcagcagcaacagcagcaacaacaaccgccgcccccgtccgccgccagcaaggcctccccgctcggcgtgcctcgtcacggtcaacgcacaattttcaacgtcgaggtccggcgtccctcgtccttcgcgtcggcggcagagcaacaacagcaccagctggccgtgctgcgggcggactgcgagctcgtcatcatccagcgcgcggaggccgcccagggcccaccagcccccgaggagcatacgtccgccggtgccgctgccgctcgcgggccagcggctgggggtgctgaggcggcggaggcggctgcccccgtgccgtgcgacgaggtggtgacgctggtccccgccttctttttctgctgctcgtccgggggtcgcgtgaccgtgcgcctgcgcccaggccgcgacggttacgtggctggcgaggccgctgaggtcgtggtggaggtggacaaccggagcaaccaggagttccgtgacgtgcgcctggaggtcgagcgccgcctcacgctggtgtcgaactcggcgggtggcggcggctcggcggggtcctcgggctcgggcagctcgtccgctacggccggtctggtgccaggctgcttcacggaggaggagcggatcttcaagtcgaagacgacagcgtgttacctgggcgcgaacgccctgcgcctgccggtccccctgcccagcaacaccccgccttccacctcgggcgcgctggtgcgttgcagctataccgcgaccgtcgaggtgctgccggcgagcgcgacggcgctgcgtggggccgctcccccgcgtctccgtgtgccgctgaccgtgttcgcgtccgcgccttcgtcgttcgccaccgccgcagcccgcaccgcgaccctgcaacaggacgccagcgagcaggcaccggccaccgtcctggtggtggtgccgcccgtggacgtggtgctgccagccgccgcaccccagctgcctcccaccgcggaggtgaacgtgaagcagcacaacggcgtggcgggcgccaaccccatgtacgccggtcccaccggtgactacaaggacgacgacgacaagcacaaccaccgccataagcacaccggttga

SEQ ID NO: 19 - protein, 406 aa, Artificial Sequence
  modified protein sequence
  MISC_FEATURE (1)..(2): XhoI restriction site
  MISC_FEATURE (388)..(389): AgeI restriction site
  MISC_FEATURE (390)..(397): FLAG Tag
  MISC_FEATURE (398)..(404): MAT Tag
  MISC_FEATURE (405)..(406): AgeI restriction site
LEHMQVYGYEVVGWEEAHAKEPKLPAADPYAPSQLVTPLDSQQQQQQQQQPPPPSAASKASPLGVPRHGQRTIFNVEVRRPSSFASAAEQQQHQLAVLRADCELVIIQRAEAAQGPPAPEEHTSAGAAAARGPAAGGAEAAEAAAPVPCDEVVTLVPAFFFCCSSGGRVTVRLRPGRDGYVAGEAAEVVVEVDNRSNQEFRDVRLEVERRLTLVSNSAGGGGSAGSSGSGSSSATAGLVPGCFTEEERIFKSKTTACYLGANALRLPVPLPSNTPPSTSGALVRCSYTATVEVLPASATALRGAAPPRLRVPLTVFASAPSSFATAAARTATLQQDASEQAPAHVLVVVPPVDVVLPAAAPQLPPTAEVNVKQHNGVAGANPMYAGPTGDYKDDDDKHNHRHKHTG

SEQ ID NO: 20 - protein, 406 aa, Artificial Sequence
  modified protein sequence
  MISC_FEATURE (1)..(2): XhoI restriction site
  MISC_FEATURE (388)..(389): AgeI restriction site
  MISC_FEATURE (390)..(397): FLAG Tag
  MISC_FEATURE (398)..(404): MAT Tag
  MISC_FEATURE (405)..(406): AgeI restriction site
LEHMQVYGYEVVGWEEAHAKEPKLPAADPYAPSQLVTPLDSQQQQQQQQQPPPPSAASKASPLGVPRHGQRTIFNVEVRRPSSFASAAEQQQHQLAVLRADCELVIIQRAEAAQGPPAPEEHTSAGAAAARGPAAGGAEAAEAAAPVPCDEVVTLVPAFFFCCSSGGRVTVRLRPGRDGYVAGEAAEVVVEVDNRSNQEFRDVRLEVERRLTLVSNSAGGGGSAGSSGSGSSSATAGLVPGCFTEEERIFKSKTTACYLGANALRLPVPLPSNTPPSTSGALVRCSYTATVEVLPASATALRGAAPPRLRVPLTVFASAPSSFATAAARHAHLQQDASEQAPATVLVVVPPVDVVLPAAAPQLPPTAEVNVKQHNGVAGANPMYAGPTGDYKDDDDKHNHRHKHTG

SEQ ID NO: 21 - protein, 406 aa, Artificial Sequence
  modified protein sequence
  MISC_FEATURE (1)..(2): XhoI restriction site
  MISC_FEATURE (388)..(389): AgeI restriction site
  MISC_FEATURE (390)..(397): FLAG Tag
  MISC_FEATURE (398)..(404): MAT Tag
  MISC_FEATURE (405)..(406): AgeI restriction site
LEHMQVYGYEVVGWEEAHAKEPKLPAADPYAPSQLVTPLDSQQQQQQQQQPPPPSAASKASPLGVPRHGQRTIFNVEVRRPSSFASAAEQQQHQLAVLRADCELVIIQRAEAAQGPPAPEEHTSAGAAAARGPAAGGAEAAEAAAPVPCDEVVTLVPAFFFCCSSGGRVTVRLRPGRDGYVAGEAAEVVVEVDNRSNQEFRDVRLEVERRLTLVSNSAGGGGSAGSSGSGSSSATAGLVPGCFTEEERIFKSKTTACYLGANALRLPVPLPSNTPPSTSGALVRCSYTATVEVLPASATALRGAAPPRLRVPLTVFASAPSSFATAAARTATLQQDASEQAPATVLVVVPPVDVVLPAAAPQLPPTAEVNVKQHNGVAGANPMYAGPTGDYKDDDDKHNHRHKHTG

SEQ ID NO: 22 - DNA, 21 bp, Artificial Sequence
  PCR primer
gacttctacgtgtgcctggag

SEQ ID NO: 23 - DNA, 21 bp, Artificial Sequence
  PCR primer
catctgtcatcaccagcctct

SEQ ID NO: 24 - DNA, 24 bp, Artificial Sequence
  PCR primer
tatgcaagtgtatggttacgaggt

SEQ ID NO: 25 - DNA, 20 bp, Artificial Sequence
  PCR primer
ggttgttgttgctgctgttg

SEQ ID NO: 26 - DNA, 19 bp, Artificial Sequence
  PCR primer
ctcacgctggtgtcgaact

SEQ ID NO: 27 - DNA, 21 bp, Artificial Sequence
  PCR primer
aggtaacacgctgtcgtcttc

SEQ ID NO: 28 - DNA, 19 bp, Artificial Sequence
  PCR primer
ggaggtgaacgtgaagcag

SEQ ID NO: 29 - DNA, 20 bp, Artificial Sequence
  PCR primer
tcctcaaccggtgtgcttat

SEQ ID NO: 30 - DNA, 19 bp, Artificial Sequence
  PCR primer
cgagctcgtcatcatccag

SEQ ID NO: 31 - DNA, 18 bp, Artificial Sequence
  PCR primer
gaccagcgtcaccacctc

SEQ ID NO: 32 - DNA, 24 bp, Artificial Sequence
  PCR Primer
agtcccatatttacacaagggcta

SEQ ID NO: 33 - DNA, 24 bp, Artificial Sequence
  PCR primer
tttcaaccaaaatgatatgcagtc

SEQ ID NO: 34 - DNA, 30 bp, Artificial Sequence
  PCR primer
taccgtactcaccgtgcgagatactgctgc

SEQ ID NO: 35 - DNA, 30 bp, Artificial Sequence
  PCR primer
cgcgcgcaaaaggctacttcccctctacgg

SEQ ID NO: 36 - DNA, 30 bp, Artificial Sequence
  PCR primer
ctctacgggcccgcgtacatcgggtttgcg

SEQ ID NO: 37 - DNA, 30 bp, Artificial Sequence
  PCR primer
atgcaggtgtatgggtacgaggtcgtgggc

SEQ ID NO: 38 - DNA, 21 bp, Chlamydomonas reinhardtii
tgtataatcacgagctcccaa

SEQ ID NO: 39 - DNA, 21 bp, Chlamydomonas reinhardtii
ttcttcagtgaagcatccctg

SEQ ID NO: 40 - DNA, 21 bp, Chlamydomonas reinhardtii
ttttgacatttacctcggcag

SEQ ID NO: 41 - DNA, 21 bp, Chlamydomonas reinhardtii
tacattgaagatggttcgctg

SEQ ID NO: 42 - DNA, 20 bp, Artificial Sequence
  PCR primer
ggtcgtgtccacgaacttcc

SEQ ID NO: 43 - DNA, 19 bp, Artificial Sequence
  PCR primer
ctcacgctggtgtcgaact

SEQ ID NO: 44 - DNA, 34 bp, Artificial Sequence
  PCR Primer
gactattaatggtgttgggtcggtgtttttggtc

SEQ ID NO: 45 - DNA, 28 bp, Artificial Sequence
  PCR Primer
agatctcagctggaacactgcgcccagg

SEQ ID NO: 46 - DNA, 42 bp, Artificial Sequence
  PCR Primer
gcagtgttccagctgagatctagccggaacactgccaggaag

SEQ ID NO: 47 - DNA, 33 bp, Artificial Sequence
  PCR Primer
gactggatccggtgtaactaagccagcccaaac

SEQ ID NO: 48 - DNA, 714 bp, Trichoderma reesei
atggtcgagtcgtgccgcccggctgcggaggtggagtcggtggccgtggagaagcgccagacgattcagccgggcaccggctacaacaacggctatttctactcctactggaacgacggccacggtggcgtcacctacaccaacggccccgggggtcagttcagcgtgaactggtcgaactccggcaacttcgtgggtggcaagggttggcagcccggcacgaagaacaaggtgatcaacttcagcggcagctacaaccctaacggcaacagctacctgtccgtgtacggttggtcccgcaaccctctcatcgagtactacatcgtggagaacttcggcacctacaatccgagcaccggcgcgacaaagctgggcgaggtcacctcggacggcagcgtgtacgacatctaccgcacacagcgcgtcaaccagccctcgatcatcggcacggcaacgttctaccagtattggtccgtgcggcggaatcaccgcagctccggttcggtgaatacggccaaccatttcaacgcttgggcccagcagggcctgacgctgggcacaatggactaccagatcgtggcggtggagggttacttcagctcgggctcggccagcatcactgtgagcaccggtgactacaaggacgacgacgacaagtccggcgagaacctgtactttcaggggcacaaccaccgccataagcacaccggttaa

SEQ ID NO: 49 - DNA, 18 bp, Artificial Sequence
  PCR Primer
cgcagccgggcggcacga

SEQ ID NO: 50 - DNA, 1215 bp, Artificial Sequence
  codon optimized
  misc_feature (1)..(6): XhoI restriction site
  misc_feature (1156)..(1161): AgeI restriction site
  misc_feature (1162)..(1185): FLAG Tag
  misc_feature (1186)..(1206): MAT Tag
  misc_feature (1207)..(1212): AgeI restriction site
ctcgagcaagtgtatggttacgaggtggtgggttgggaggaggctcatgctaaggagcccaagctgcccgcggccgacccctacgccccatcccaactggtcactccgctggacagccagcagcagcaacagcagcaacaacaaccgccgcccccgtccgccgccagcaaggcctccccgctcggcgtgcctcgtcacggtcaacgcacaattttcaacgtcgaggtccggcgtccctcgtccttcgcgtcggcggcagagcaacaacagcaccagctggccgtgctgcgggcggactgcgagctcgtcatcatccagcgcgcggaggccgcccagggcccaccagcccccgaggagcatacgtccgccggtgccgctgccgctcgcgggccagcggctgggggtgctgaggcggcggaggcggctgcccccgtgccgtgcgacgaggtggtgacgctggtccccgccttctttttctgctgctcgtccgggggtcgcgtgaccgtgcgcctgcgcccaggccgcgacggttacgtggctggcgaggccgctgaggtcgtggtggaggtggacaaccggagcaaccaggagttccgtgacgtgcgcctggaggtcgagcgccgcctcacgctggtgtcgaactcggcgggtggcggcggctcggcggggtcctcgggctcgggcagctcgtccgctacggccggtctggtgccaggctgcttcacggaggaggagcggatcttcaagtcgaagacgacagcgtgttacctgggcgcgaacgccctgcgcctgccggtccccctgcccagcaacaccccgccttccacctcgggcgcgctggtgcgttgcagctataccgcgaccgtcgaggtgctgccggcgagcgcgacggcgctgcgtggggccgctcccccgcgtctccgtgtgccgctgaccgtgttcgcgtccgcgccttcgtcgttcgccaccgccgcagcccgccacgcgcacctgcaacaggacgccagcgagcaggcaccggcccacgtcctggtggtggtgccgcccgtggacgtggtgctgccagccgccgcaccccagctgcctcccaccgcggaggtgaacgtgaagcagcacaacggcgtggcgggcgccaaccccatgtacgccggtcccaccggtgactacaaggacgacgacgacaagcacaaccaccgccataagcacaccggttga

SEQ ID NO: 51 - protein, 383 aa, Chlamydomonas reinhardtii
QVYGYEVVGWEEAHAKEPKLPAADPYAPSQLVTPLDSQQQQQQQQQPPPPSAASKASPLGVPRHGQRTIFNVEVRRPSSFASAAEQQQHQLAVLRADCELVIIQRAEAAQGPPAPEEHTSAGAAAARGPAAGGAEAAEAAAPVPCDEVVTLVPAFFFCCSSGGRVTVRLRPGRDGYVAGEAAEVVVEVDNRSNQEFRDVRLEVERRLTLVSNSAGGGGSAGSSGSGSSSATAGLVPGCFTEEERIFKSKTTACYLGANALRLPVPLPSNTPPSTSGALVRCSYTATVEVLPASATALRGAAPPRLRVPLTVFASAPSSFATAAARHAHLQQDASEQAPAHVLVVVPPVDVVLPAAAPQLPPTAEVNVKQHNGVAGANPMYAGP

SEQ ID NO: 52 - protein, 450 aa, Chlamydomonas reinhardtii
QVYGYEVVGWEEAHAKEPKLPAADPYAPSQLVTPLDSQQQQQQQQQPPPPSAASKASPLGVPRHGQRTIFNVCVPLLAGGRQVLPPGTYRLPFRLQLPADLPGTFRLAGTPARTIGDVSYRNLSGEVSYGLQVEVRRPSSFASAAEQQQHQLAVLRADCELVIIQRAEAAQGPPAPEEHTSAGAAAARGPAAGGAEAAEAAAPVPCDEVVTLVPAFFFCCSSGGRVTVRLRPGRDGYVAGEAAEVVVEVDNRSNQEFRDVRLEVERRLTLVSNSAGGGGSAGSSGSGSSSATAGLVPGCFTEEERIFKSKTTAALLPGACYLGANALRLPVPLPSNTPPSTSGALVRCSYTATVEVLPASATALRGAAPPRLRVPLTVFASAPSSFATAAARHAHLQQDASEQAPAHVLVVVPPVDVVLPAAAPQLPPTAEVNVKQHNGVAGANPMYAGP

SEQ ID NO: 53 - DNA, 1149 bp, Artificial Sequence
  codon optimized
caagtgtatggttacgaggtggtgggttgggaggaggctcatgctaaggagcccaagctgcccgcggccgacccctacgccccatcccaactggtcactccgctggacagccagcagcagcaacagcagcaacaacaaccgccgcccccgtccgccgccagcaaggcctccccgctcggcgtgcctcgtcacggtcaacgcacaattttcaacgtcgaggtccggcgtccctcgtccttcgcgtcggcggcagagcaacaacagcaccagctggccgtgctgcgggcggactgcgagctcgtcatcatccagcgcgcggaggccgcccagggcccaccagcccccgaggagcatacgtccgccggtgccgctgccgctcgcgggccagcggctgggggtgctgaggcggcggaggcggctgcccccgtgccgtgcgacgaggtggtgacgctggtccccgccttctttttctgctgctcgtccgggggtcgcgtgaccgtgcgcctgcgcccaggccgcgacggttacgtggctggcgaggccgctgaggtcgtggtggaggtggacaaccggagcaaccaggagttccgtgacgtgcgcctggaggtcgagcgccgcctcacgctggtgtcgaactcggcgggtggcggcggctcggcggggtcctcgggctcgggcagctcgtccgctacggccggtctggtgccaggctgcttcacggaggaggagcggatcttcaagtcgaagacgacagcgtgttacctgggcgcgaacgccctgcgcctgccggtccccctgcccagcaacaccccgccttccacctcgggcgcgctggtgcgttgcagctataccgcgaccgtcgaggtgctgccggcgagcgcgacggcgctgcgtggggccgctcccccgcgtctccgtgtgccgctgaccgtgttcgcgtccgcgccttcgtcgttcgccaccgccgcagcccgcaccgcgaccctgcaacaggacgccagcgagcaggcaccggcccacgtcctggtggtggtgccgcccgtggacgtggtgctgccagccgccgcaccccagctgcctcccaccgcggaggtgaacgtgaagcagcacaacggcgtggcgggcgccaaccccatgtacgccggtccc

SEQ ID NO: 54 - DNA, 1149 bp, Artificial Sequence
  codon optimized
caagtgtatggttacgaggtggtgggttgggaggaggctcatgctaaggagcccaagctgcccgcggccgacccctacgccccatcccaactggtcactccgctggacagccagcagcagcaacagcagcaacaacaaccgccgcccccgtccgccgccagcaaggcctccccgctcggcgtgcctcgtcacggtcaacgcacaattttcaacgtcgaggtccggcgtccctcgtccttcgcgtcggcggcagagcaacaacagcaccagctggccgtgctgcgggcggactgcgagctcgtcatcatccagcgcgcggaggccgcccagggcccaccagcccccgaggagcatacgtccgccggtgccgctgccgctcgcgggccagcggctgggggtgctgaggcggcggaggcggctgcccccgtgccgtgcgacgaggtggtgacgctggtccccgccttctttttctgctgctcgtccgggggtcgcgtgaccgtgcgcctgcgcccaggccgcgacggttacgtggctggcgaggccgctgaggtcgtggtggaggtggacaaccggagcaaccaggagttccgtgacgtgcgcctggaggtcgagcgccgcctcacgctggtgtcgaactcggcgggtggcggcggctcggcggggtcctcgggctcgggcagctcgtccgctacggccggtctggtgccaggctgcttcacggaggaggagcggatcttcaagtcgaagacgacagcgtgttacctgggcgcgaacgccctgcgcctgccggtccccctgcccagcaacaccccgccttccacctcgggcgcgctggtgcgttgcagctataccgcgaccgtcgaggtgctgccggcgagcgcgacggcgctgcgtggggccgctcccccgcgtctccgtgtgccgctgaccgtgttcgcgtccgcgccttcgtcgttcgccaccgccgcagcccgccacgcgcacctgcaacaggacgccagcgagcaggcaccggccaccgtcctggtggtggtgccgcccgtggacgtggtgctgccagccgccgcaccccagctgcctcccaccgcggaggtgaacgtgaagcagcacaacggcgtggcgggcgccaaccccatgtacgccggtccc

SEQ ID NO: 55 - DNA, 1149 bp, Artificial Sequence
  codon optimized
caagtgtatggttacgaggtggtgggttgggaggaggctcatgctaaggagcccaagctgcccgcggccgacccctacgccccatcccaactggtcactccgctggacagccagcagcagcaacagcagcaacaacaaccgccgcccccgtccgccgccagcaaggcctccccgctcggcgtgcctcgtcacggtcaacgcacaattttcaacgtcgaggtccggcgtccctcgtccttcgcgtcggcggcagagcaacaacagcaccagctggccgtgctgcgggcggactgcgagctcgtcatcatccagcgcgcggaggccgcccagggcccaccagcccccgaggagcatacgtccgccggtgccgctgccgctcgcgggccagcggctgggggtgctgaggcggcggaggcggctgcccccgtgccgtgcgacgaggtggtgacgctggtccccgccttctttttctgctgctcgtccgggggtcgcgtgaccgtgcgcctgcgcccaggccgcgacggttacgtggctggcgaggccgctgaggtcgtggtggaggtggacaaccggagcaaccaggagttccgtgacgtgcgcctggaggtcgagcgccgcctcacgctggtgtcgaactcggcgggtggcggcggctcggcggggtcctcgggctcgggcagctcgtccgctacggccggtctggtgccaggctgcttcacggaggaggagcggatcttcaagtcgaagacgacagcgtgttacctgggcgcgaacgccctgcgcctgccggtccccctgcccagcaacaccccgccttccacctcgggcgcgctggtgcgttgcagctataccgcgaccgtcgaggtgctgccggcgagcgcgacggcgctgcgtggggccgctcccccgcgtctccgtgtgccgctgaccgtgttcgcgtccgcgccttcgtcgttcgccaccgccgcagcccgcaccgcgaccctgcaacaggacgccagcgagcaggcaccggccaccgtcctggtggtggtgccgcccgtggacgtggtgctgccagccgccgcaccccagctgcctcccaccgcggaggtgaacgtgaagcagcacaacggcgtggcgggcgccaaccccatgtacgccggtccc

SEQ ID NO: 56 - protein, 385 aa, Artificial Sequence
  modified protein sequence
HMQVYGYEVVGWEEAHAKEPKLPAADPYAPSQLVTPLDSQQQQQQQQQPPPPSAASKASPLGVPRHGQRTIFNVEVRRPSSFASAAEQQQHQLAVLRADCELVIIQRAEAAQGPPAPEEHTSAGAAAARGPAAGGAEAAEAAAPVPCDEVVTLVPAFFFCCSSGGRVTVRLRPGRDGYVAGEAAEVVVEVDNRSNQEFRDVRLEVERRLTLVSNSAGGGGSAGSSGSGSSSATAGLVPGCFTEEERIFKSKTTACYLGANALRLPVPLPSNTPPSTSGALVRCSYTATVEVLPASATALRGAAPPRLRVPLTVFASAPSSFATAAARTATLQQDASEQAPAHVLVVVPPVDVVLPAAAPQLPPTAEVNVKQHNGVAGANPMYAGP

SEQ ID NO: 57 - protein, 385 aa, Artificial Sequence
  modified protein sequence
HMQVYGYEVVGWEEAHAKEPKLPAADPYAPSQLVTPLDSQQQQQQQQQPPPPSAASKASPLGVPRHGQRTIFNVEVRRPSSFASAAEQQQHQLAVLRADCELVIIQRAEAAQGPPAPEEHTSAGAAAARGPAAGGAEAAEAAAPVPCDEVVTLVPAFFFCCSSGGRVTVRLRPGRDGYVAGEAAEVVVEVDNRSNQEFRDVRLEVERRLTLVSNSAGGGGSAGSSGSGSSSATAGLVPGCFTEEERIFKSKTTACYLGANALRLPVPLPSNTPPSTSGALVRCSYTATVEVLPASATALRGAAPPRLRVPLTVFASAPSSFATAAARHAHLQQDASEQAPATVLVVVPPVDVVLPAAAPQLPPTAEVNVKQHNGVAGANPMYAGP

SEQ ID NO: 58 - protein, 385 aa, Artificial Sequence
  modified protein sequence
HMQVYGYEVVGWEEAHAKEPKLPAADPYAPSQLVTPLDSQQQQQQQQQPPPPSAASKASPLGVPRHGQRTIFNVEVRRPSSFASAAEQQQHQLAVLRADCELVIIQRAEAAQGPPAPEEHTSAGAAAARGPAAGGAEAAEAAAPVPCDEVVTLVPAFFFCCSSGGRVTVRLRPGRDGYVAGEAAEVVVEVDNRSNQEFRDVRLEVERRLTLVSNSAGGGGSAGSSGSGSSSATAGLVPGCFTEEERIFKSKTTACYLGANALRLPVPLPSNTPPSTSGALVRCSYTATVEVLPASATALRGAAPPRLRVPLTVFASAPSSFATAAARTATLQQDASEQAPATVLVVVPPVDVVLPAAAPQLPPTAEVNVKQHNGVAGANPMYAGP

SEQ ID NO: 59 - DNA, 500 bp, Chlamydomonas reinhardtii
aggggaagtagccttttgcgcgcgtcgttcgggccgcggcgcttgcagcagtatctcgcacggtgagtacggtattccgattcccggcagtcgcagaagcgtgatgaaacaggcaatagcaggtatcgaaacggcctgcggttgcgtggaagccgctgcgctgttgtgtgatgcattgttaagttgcatgcatagcccttgtgtaaatatgggactgcatatcattttggttgaaagggcagagggacgaccctgtgggtgcctcgggtcacggcgtggccgaggtgcacccttgctgcgtaggaaggcgtgtggcgtgccttcggacggcacgcagggcggttgaagtaaggcactgggtcgtggtgtgttcatttatgcgctccttcaagtatcctgcttatttgatgcgtgtttgattgctagcattagcaatatgtactgtgaggcctactttgctcgctgcacaccgcacacatggacggacggaattatggtct

SEQ ID NO: 60 - DNA, 500 bp, Chlamydomonas reinhardtii
ggaataacgacctgacgcgagcggggcaagaggcgccttcatgccgctactgcaagtggtcatgaagacagtcgcttctcgcgagacagcccaaatgatatcaataagatgttacaaggccgatacctgaagctgcagctcaagagcgtgccaaagggtcgctcccccctccccactcagtctggaacgggcagcttcgagcgccttcaatatgccttcggggttgccgttaggcgccttgcctaccgggctacaagggcgctctcatcttagcggcgtgatccctcagatgtgcaagggggaaacgcaccggggggcgggggcggcaggccgctgccaactgtgcctgctggcctgctggccctgtcaacgggtgtgcgtgctggttggtatacgaaccggcgcgggctgcggcgtgttcacgtgagcggctcccgcatgcacccaacgtcgcccccttttctgttttctgcctgccgcccgtgattgatgcccgtggc

SEQ ID NO: 61 - DNA, 768 bp, Chlamydomonas reinhardtii
atgacggcgttcctgttattcagcaacgctatgcgggcagccgtcaaggcggagagccctggcattgatttcggcgaggtcagcaagatcctcggcgagaaatgggcgagaatatgtgcaaaggagaaggcggagtacgaggcaaaggcggcggaggacaaggatcgctatctgcgggagatgcaggaatatgcgagcaccaagagcgacagcgagagtgaggctcggtcgccctcaggcaagaagcacaagggcggccatgtgaaggcctcagcggctcaagcatacgcgcaggagtggcgcaaggagcctgccgtggagtctgcgcgcctgggaggcaacgagcgcaaggcctccggcgctcccaagaagcccatgacgccgttcctgcatttcagcaacgcggtgcgggagtccgtcaaggccgagaaccctggcatcgcctttggcgagctcgccaaggtcatcggcgagaaatgggcgaagctgagtgcacaggagaaggcggagtacgtgaagcggttcgatgaggataagcagcgctatgcgcgggagatgcaggactatgccttagccagggagggcgccgccggctctgggagcgcggccgctgatgcgcccatacgggtggaggaggagacgacggaggaggaggacgagacggatgtggaggaggaggaggaggaggaggaggagaaggtggaggaggaggaggaggaggaggagcagcgggaagaggagtacaggagcaatgagtggcaggtgtga

SEQ ID NO: 62 - protein, 255 aa, Chlamydomonas reinhardtii
MTAFLLFSNAMRAAVKAESPGIDFGEVSKILGEKWARICAKEKAEYEAKAAEDKDRYLREMQEYASTKSDSESEARSPSGKKHKGGHVKASAAQAYAQEWRKEPAVESARLGGNERKASGAPKKPMTPFLHFSNAVRESVKAENPGIAFGELAKVIGEKWAKLSAQEKAEYVKRFDEDKQRYAREMQDYALAREGAAGSGSAAADAPIRVEEETTEEEDETDVEEEEEEEEEKVEEEEEEEEQREEEYRSNEWQV

SEQ ID NO: 63 - DNA, 774 bp, artificial sequence
  codon optimized
catatgactgctttcctgctgttttccaatgcaatgcgcgctgctgtcaaggctgagtcgccgggcatcgactttggcgaggtgtccaagatcctcggcgagaagtgggcccgtatctgcgctaaggagaaggccgagtacgaggcgaaggccgccgaggacaaggaccgctacctgcgtgagatgcaggagtacgcttcgaccaagtccgacagcgagagcgaggcccgctcccccagcggtaagaagcacaagggcggccacgtgaaggcctcggcggcccaagcctatgcgcaggagtggcgcaaggagcccgcggtggagagcgcccgcctgggtggcaacgagcgcaaggcgagcggtgcgcccaagaagcccatgaccccgttcctccacttctcgaacgcggtgcgcgagtcggtcaaggcggagaacccgggcatcgccttcggcgagctggcaaaggtcatcggcgagaagtgggccaagctcagcgctcaggagaaggcggagtacgtgaagcgcttcgacgaggacaagcagcgctatgcccgcgagatgcaggactatgccctggcccgggagggcgcggccggctccggctccgcggctgccgacgcccccattcgtgtggaggaggagacaactgaggaggaggacgagacagacgtggaggaggaggaggaggaggaggaggagaaggtcgaggaggaggaggaggaggaggagcagcgggaggaggagtatcggtccaacgagtggcaggtgaccggt

SEQ ID NO: 64 - DNA, 762 bp, artificial sequence
  codon optimized
actgctttcctgctgttttccaatgcaatgcgcgctgctgtcaaggctgagtcgccgggcatcgactttggcgaggtgtccaagatcctcggcgagaagtgggcccgtatctgcgctaaggagaaggccgagtacgaggcgaaggccgccgaggacaaggaccgctacctgcgtgagatgcaggagtacgcttcgaccaagtccgacagcgagagcgaggcccgctcccccagcggtaagaagcacaagggcggccacgtgaaggcctcggcggcccaagcctatgcgcaggagtggcgcaaggagcccgcggtggagagcgcccgcctgggtggcaacgagcgcaaggcgagcggtgcgcccaagaagcccatgaccccgttcctccacttctcgaacgcggtgcgcgagtcggtcaaggcggagaacccgggcatcgccttcggcgagctggcaaaggtcatcggcgagaagtgggccaagctcagcgctcaggagaaggcggagtacgtgaagcgcttcgacgaggacaagcagcgctatgcccgcgagatgcaggactatgccctggcccgggagggcgcggccggctccggctccgcggctgccgacgcccccattcgtgtggaggaggagacaactgaggaggaggacgagacagacgtggaggaggaggaggaggaggaggaggagaaggtcgaggaggaggaggaggaggaggagcagcgggaggaggagtatcggtccaacgagtggcaggtg

SEQ ID NO: 65 - DNA, 762 bp, Chlamydomonas reinhardtii
acggcgttcctgttattcagcaacgctatgcgggcagccgtcaaggcggagagccctggcattgatttcggcgaggtcagcaagatcctcggcgagaaatgggcgagaatatgtgcaaaggagaaggcggagtacgaggcaaaggcggcggaggacaaggatcgctatctgcgggagatgcaggaatatgcgagcaccaagagcgacagcgagagtgaggctcggtcgccctcaggcaagaagcacaagggcggccatgtgaaggcctcagcggctcaagcatacgcgcaggagtggcgcaaggagcctgccgtggagtctgcgcgcctgggaggcaacgagcgcaaggcctccggcgctcccaagaagcccatgacgccgttcctgcatttcagcaacgcggtgcgggagtccgtcaaggccgagaaccctggcatcgcctttggcgagctcgccaaggtcatcggcgagaaatgggcgaagctgagtgcacaggagaaggcggagtacgtgaagcggttcgatgaggataagcagcgctatgcgcgggagatgcaggactatgccttagccagggagggcgccgccggctctgggagcgcggccgctgatgcgcccatacgggtggaggaggagacgacggaggaggaggacgagacggatgtggaggaggaggaggaggaggaggaggagaaggtggaggaggaggaggaggaggaggagcagcgggaagaggagtacaggagcaatgagtggcaggtg

SEQ ID NO: 66 - protein, 254 aa, Chlamydomonas reinhardtii
TAFLLFSNAMRAAVKAESPGIDFGEVSKILGEKWARICAKEKAEYEAKAAEDKDRYLREMQEYASTKSDSESEARSPSGKKHKGGHVKASAAQAYAQEWRKEPAVESARLGGNERKASGAPKKPMTPFLHFSNAVRESVKAENPGIAFGELAKVIGEKWAKLSAQEKAEYVKRFDEDKQRYAREMQDYALAREGAAGSGSAAADAPIRVEEETTEEEDETDVEEEEEEEEEKVEEEEEEEEQREEEYRSNEWQV

SEQ ID NO: 67 - DNA, 1155 bp, Chlamydomonas reinhardtii; identical to SEQ ID NO: 1

SEQ ID NO: 68 - protein, 384 aa, Chlamydomonas reinhardtii; identical to SEQ ID NO: 6

SEQ ID NO: 69 - DNA, 1161 bp, artificial sequence
  codon optimized
catatgcaagtgtatggttacgaggtggtgggttgggaggaggctcatgctaaggagcccaagctgcccgcggccgacccctacgccccatcccaactggtcactccgctggacagccagcagcagcaacagcagcaacaacaaccgccgcccccgtccgccgccagcaaggcctccccgctcggcgtgcctcgtcacggtcaacgcacaattttcaacgtcgaggtccggcgtccctcgtccttcgcgtcggcggcagagcaacaacagcaccagctggccgtgctgcgggcggactgcgagctcgtcatcatccagcgcgcggaggccgcccagggcccaccagcccccgaggagcatacgtccgccggtgccgctgccgctcgcgggccagcggctgggggtgctgaggcggcggaggcggctgcccccgtgccgtgcgacgaggtggtgacgctggtccccgccttctttttctgctgctcgtccgggggtcgcgtgaccgtgcgcctgcgcccaggccgcgacggttacgtggctggcgaggccgctgaggtcgtggtggaggtggacaaccggagcaaccaggagttccgtgacgtgcgcctggaggtcgagcgccgcctcacgctggtgtcgaactcggcgggtggcggcggctcggcggggtcctcgggctcgggcagctcgtccgctacggccggtctggtgccaggctgcttcacggaggaggagcggatcttcaagtcgaagacgacagcgtgttacctgggcgcgaacgccctgcgcctgccggtccccctgcccagcaacaccccgccttccacctcgggcgcgctggtgcgttgcagctataccgcgaccgtcgaggtgctgccggcgagcgcgacggcgctgcgtggggccgctcccccgcgtctccgtgtgccgctgaccgtgttcgcgtccgcgccttcgtcgttcgccaccgccgcagcccgccacgcgcacctgcaacaggacgccagcgagcaggcaccggcccacgtcctggtggtggtgccgcccgtggacgtggtgctgccagccgccgcaccccagctgcctcccaccgcggaggtgaacgtgaagcagcacaacggcgtggcgggcgccaaccccatgtacgccggtcccaccggt

SEQ ID NO: 70 - DNA, 1149 bp, artificial sequence; identical to SEQ ID NO: 4
  codon optimized

SEQ ID NO: 71 - DNA, 1149 bp, Chlamydomonas reinhardtii; identical to SEQ ID NO: 2

SEQ ID NO: 72 - protein, 383 aa, Chlamydomonas reinhardtii; identical to SEQ ID NO: 51

SEQ ID NO: 73 - DNA, 1230 bp, Chlamydomonas reinhardtii
atggtgtatttatgcgaatcgcagacgcttgacttgcagttcaagattgagttctccatcctcagcgtgggcaccgtgttcccgctggtgttcagcatccagcaggccttctcccggcgtgaggaggcgctgagagacctgtccatgctcaagtcgcaactcatggccctgtacttcgccaaccgcgactgggacttccacgacggcgccgcggtgcgctccaacctgggcaacgacgaggcgccgcacgcggtggccagtgcccggctgatcgtgacgctgctggcgaccatgaagaaatggctgtcgggcgcgttcttccgtgggaaagagggccgccgggatgtggacgacagcagtgacgacgacagcggcgatgagggcgatgaggtcgctgagggcggcaaggacgcgggcggcgagggcggcggcggcggcggcggcggcgaggagcggttgacggctgaggacattgccgaggcggacccgcactggcagttgtactacgagatgtacgacatcatctcccagatccaacttaacaacgaagccatgacggtgccggcgggctgctccaagggaggcaagtgcgagggcggcatgagccgcatggcgggctacgtggagaagatagtcgagatggtggagaggctgcggcatctgcgcgagtaccgcacgcccttcatgcttcgctacgtgtcgttcacgctggtgtgcgcctcaatcttcctggccgcgccctacttcgcctggctgtgcgagggcacgcgctgggacggcgacagctcgggccgctgccccgccggctacttcacaggggtgctgtacgtgctggtggtgtccacgctttttcacgtgcaggtggctctggagaacccgtttgacggcgtggggctggatgacgtgttcttcaacatggaccgggagttcgcggtcactgtgcgcagtgccgaaaaggacgccgccggcgggggcagcggccgtgattcgggtggggacccgcgcgcgccggcagacgcggtgggaaaggtggcggcgctggcctccgccgccaccactgctccggtgaaagccgtggccacgcttgctgtgggcggtggcgaggcaggcggccgcgttgtggctggcacgacggcaccggtggcggcactggtgctgccggcagcggttgagcgagcgaacacgctgaccccggcgacagtggtggaggactgggatggtcgggtgtag

SEQ ID NO: 74 - protein, 409 aa, Chlamydomonas reinhardtii
MVYLCESQTLDLQFKIEFSILSVGTVFPLVFSIQQAFSRREEALRDLSMLKSQLMALYFANRDWDFHDGAAVRSNLGNDEAPHAVASARLIVTLLATMKKWLSGAFFRGKEGRRDVDDSSDDDSGDEGDEVAEGGKDAGGEGGGGGGGGEERLTAEDIAEADPHWQLYYEMYDIISQIQLNNEAMTVPAGCSKGGKCEGGMSRMAGYVEKIVEMVERLRHLREYRTPFMLRYVSFTLVCASIFLAAPYFAWLCEGTRWDGDSSGRCPAGYFTGVLYVLVVSTLFHVQVALENPFDGVGLDDVFFNMDREFAVTVRSAEKDAAGGGSGRDSGGDPRAPADAVGKVAALASAATTAPVKAVATLAVGGGEAGGRVVAGTTAPVAALVLPAAVERANTLTPATVVEDWDGRV

SEQ ID NO: 75 - DNA, 1251 bp, artificial sequence
  codon optimized
catatgctcgaggtgtacctgtgtgagtcccaaaccctcgacctccaatttaagattgagttctccatcctgtccgtgggcaccgtgtttccgctcgtgttcagcatccagcaagcgtttagccgccgtgaggaggcgctgcgcgacctgtccatgctcaagagccagctgatggccctgtactttgccaaccgcgactgggatttccacgacggcgcagccgtgcgcagcaacctgggcaacgacgaggcgccccatgccgtggctagcgcgcgcctcatcgtgaccctgctggccaccatgaagaagtggctgtccggcgccttcttccggggcaaggagggccgccgcgacgtggacgactcgtcggacgacgacagcggggacgagggcgacgaggtggccgagggcggcaaggacgcaggcggggagggtggtggtggcggtgggggtggcgaggagcgcctgacggctgaggacatcgccgaggcggacccccactggcagctgtactacgagatgtacgacatcatttcccagatccagctgaacaacgaggcgatgacggtgcctgccgggtgcagcaagggtggcaagtgcgagggcggcatgtcccggatggccggctatgtggagaagatcgtcgagatggtcgagcgtctccgccacctccgtgagtaccggaccccgtttatgctgcggtacgtctcgttcaccctggtgtgcgcctccattttcctcgctgccccatacttcgcgtggctgtgcgagggcacccgctgggacggcgactccagcgggcgttgccccgcaggttacttcacgggcgtgctgtatgtcctggtcgtgagcaccctgttccacgtgcaggtcgcactggagaacccattcgacggcgtgggcctggacgacgtgttcttcaacatggaccgtgagtttgcggtgaccgtccgctcggcggagaaggacgcagcgggcgggggcagcggccgtgattccggcggtgacccacgcgctcccgccgacgccgtggggaaggtggccgcgctggcgtcggctgcaacgactgcacccgtgaaggccgtggctacgctggccgtgggcggtggcgaggcaggcggtcgcgtggtggccggcaccacggctcctgtggccgccctggtgctccctgcggcagtggagcgcgcgaacaccctgacccccgcgaccgtggtggaggattgggacggccgcgtcaccggttagggatcc

SEQ ID NO: 76 - DNA, 1224 bp, artificial sequence
  codon optimized
gtgtacctgtgtgagtcccaaaccctcgacctccaatttaagattgagttctccatcctgtccgtgggcaccgtgtttccgctcgtgttcagcatccagcaagcgtttagccgccgtgaggaggcgctgcgcgacctgtccatgctcaagagccagctgatggccctgtactttgccaaccgcgactgggatttccacgacggcgcagccgtgcgcagcaacctgggcaacgacgaggcgccccatgccgtggctagcgcgcgcctcatcgtgaccctgctggccaccatgaagaagtggctgtccggcgccttcttccggggcaaggagggccgccgcgacgtggacgactcgtcggacgacgacagcggggacgagggcgacgaggtggccgagggcggcaaggacgcaggcggggagggtggtggtggcggtgggggtggcgaggagcgcctgacggctgaggacatcgccgaggcggacccccactggcagctgtactacgagatgtacgacatcatttcccagatccagctgaacaacgaggcgatgacggtgcctgccgggtgcagcaagggtggcaagtgcgagggcggcatgtcccggatggccggctatgtggagaagatcgtcgagatggtcgagcgtctccgccacctccgtgagtaccggaccccgtttatgctgcggtacgtctcgttcaccctggtgtgcgcctccattttcctcgctgccccatacttcgcgtggctgtgcgagggcacccgctgggacggcgactccagcgggcgttgccccgcaggttacttcacgggcgtgctgtatgtcctggtcgtgagcaccctgttccacgtgcaggtcgcactggagaacccattcgacggcgtgggcctggacgacgtgttcttcaacatggaccgtgagtttgcggtgaccgtccgctcggcggagaaggacgcagcgggcgggggcagcggccgtgattccggcggtgacccacgcgctcccgccgacgccgtggggaaggtggccgcgctggcgtcggctgcaacgactgcacccgtgaaggccgtggctacgctggccgtgggcggtggcgaggcaggcggtcgcgtggtggccggcaccacggctcctgtggccgccctggtgctccctgcggcagtggagcgcgcgaacaccctgacccccgcgaccgtggtggaggattgggacggccgcgtc

SEQ ID NO: 77 - DNA, 1224 bp, Chlamydomonas reinhardtii
gtgtatttatgcgaatcgcagacgcttgacttgcagttcaagattgagttctccatcctcagcgtgggcaccgtgttcccgctggtgttcagcatccagcaggccttctcccggcgtgaggaggcgctgagagacctgtccatgctcaagtcgcaactcatggccctgtacttcgccaaccgcgactgggacttccacgacggcgccgcggtgcgctccaacctgggcaacgacgaggcgccgcacgcggtggccagtgcccggctgatcgtgacgctgctggcgaccatgaagaaatggctgtcgggcgcgttcttccgtgggaaagagggccgccgggatgtggacgacagcagtgacgacgacagcggcgatgagggcgatgaggtcgctgagggcggcaaggacgcgggcggcgagggcggcggcggcggcggcggcggcgaggagcggttgacggctgaggacattgccgaggcggacccgcactggcagttgtactacgagatgtacgacatcatctcccagatccaacttaacaacgaagccatgacggtgccggcgggctgctccaagggaggcaagtgcgagggcggcatgagccgcatggcgggctacgtggagaagatagtcgagatggtggagaggctgcggcatctgcgcgagtaccgcacgcccttcatgcttcgctacgtgtcgttcacgctggtgtgcgcctcaatcttcctggccgcgccctacttcgcctggctgtgcgagggcacgcgctgggacggcgacagctcgggccgctgccccgccggctacttcacaggggtgctgtacgtgctggtggtgtccacgctttttcacgtgcaggtggctctggagaacccgtttgacggcgtggggctggatgacgtgttcttcaacatggaccgggagttcgcggtcactgtgcgcagtgccgaaaaggacgccgccggcgggggcagcggccgtgattcgggtggggacccgcgcgcgccggcagacgcggtgggaaaggtggcggcgctggcctccgccgccaccactgctccggtgaaagccgtggccacgcttgctgtgggcggtggcgaggcaggcggccgcgttgtggctggcacgacggcaccggtggcggcactggtgctgccggcagcggttgagcgagcgaacacgctgaccccggcgacagtggtggaggactgggatggtcgggtg

SEQ ID NO: 78 - protein, 408 aa, Chlamydomonas reinhardtii
VYLCESQTLDLQFKIEFSILSVGTVFPLVFSIQQAFSRREEALRDLSMLKSQLMALYFANRDWDFHDGAAVRSNLGNDEAPHAVASARLIVTLLATMKKWLSGAFFRGKEGRRDVDDSSDDDSGDEGDEVAEGGKDAGGEGGGGGGGGEERLTAEDIAEADPHWQLYYEMYDIISQIQLNNEAMTVPAGCSKGGKCEGGMSRMAGYVEKIVEMVERLRHLREYRTPFMLRYVSFTLVCASIFLAAPYFAWLCEGTRWDGDSSGRCPAGYFTGVLYVLVVSTLFHVQVALENPFDGVGLDDVFFNMDREFAVTVRSAEKDAAGGGSGRDSGGDPRAPADAVGKVAALASAATTAPVKAVATLAVGGGEAGGRVVAGTTAPVAALVLPAAVERANTLTPATVVEDWDGRV

SEQ ID NO: 79 - DNA, 1371 bp, Chlamydomonas reinhardtii
atgggcgagttgagttcctccgcggcagccgagcccgccaccaacacgctgctgaacgggctgctggcgacagtggtgaagcggcagaagtctgtgtctacgcggtttcaaatgcactcccgcaaggtcaaggaacaggacgctgacgggctcctggacgaacagctgcccgacctggcggtggcgctggcgtcgctggtggcggaggcggcggcgcgggagcaggagcagcgggcagcggccggcggcggcggcggcctccgccggaacccctcttgctctcgctcaacgggaagcgacgacggcggcggccggcggtcgccgactgcccgaagtcgcagccccagctgcagccccagccgcagccctagccctggccggcggctcgctcaaaggaggacccagagcctacgcaacagcagcagcggcatggacgttccaagcaacggcaacgacgctgatgacacgggcgctgtcgccgcctcctcctcgacgctggcgccttcagcatcctttccggctcccggcgcctgcctgcctggcacagcacgccccagctccacccgcttcggccccacgcctccgacatcgggcccgccaccagcctcaaccaacagcgccagcggggcgccggcggcgtcggcgccccagccaccagctgacctgtccagcttccagcagacgcggcggcagcgcctgcaacagcagctccagcggccgcagctcagcaccggcggtggtggcccgggcaacgctgacggccttgcgccccgctcgtcctcctccttcacgcgcgtgcccgcctcccgcctggtgatgggcggcgtctcgcagtcgttggatggcggcgtctgggcgtcggcggagggcgcggcggccgctgtggtggcgctgtcaggcgaggtacggccgtactcgtcgcccttgcaggcgccgtggacccagggagacggactccagctgccgcgcctgaccagcgctggcggggcggcaagccaccgcacgcagcccgcgggcggcgttgccggggtgtcaggcgctgtggtcgatgcgagggcagcagcaggcggcggagccgcttcgcctgcgtggtcagcgcccggcggtgctgctccgacgggggagagcagcggcagcggtgtgtgtggtgccatgtcgccgcgattgcgcagcagcggcagccgcagtcgcttccaatcgccgtcatccagccgccgcctgataccgacgggtgccgctactggcgtttctggtggcggtggcagcaacggcgcgccaccatcatcgcctggcggctccgtgcctctcacacggggccagtcctggtgggctaagagcaccgtggcacgagagctggtgttcctcaaatag

SEQ ID NO: 80 - protein, 456 aa, Chlamydomonas reinhardtii
MGELSSSAAAEPATNTLLNGLLATVVKRQKSVSTRFQMHSRKVKEQDADGLLDEQLPDLAVALASLVAEAAAREQEQRAAAGGGGGLRRNPSCSRSTGSDDGGGRRSPTARSRSPSCSPSRSPSPGRRLAQRRTQSLRNSSSGMDVPSNGNDADDTGAVAASSSTLAPSASFPAPGACLPGTARPSSTRFGPTPPTSGPPPASTNSASGAPAASAPQPPADLSSFQQTRRQRLQQQLQRPQLSTGGGGPGNADGLAPRSSSSFTRVPASRLVMGGVSQSLDGGVWASAEGAAAAVVALSGEVRPYSSPLQAPWTQGDGLQLPRLTSAGGAASHRTQPAGGVAGVSGAVVDARAAAGGGAASPAWSAPGGAAPTGESSGSGVCGAMSPRLRSSGSRSRFQSPSSSRRLIPTGAATGVSGGGGSNGAPPSSPGGSVPLTRGQSWWAKSTVARELVFLK

SEQ ID NO: 81 - DNA, 1386 bp, artificial sequence
  codon optimized
catatgctcgagggtgagctctcctcctccgcagcagccgagccagccactaatacactgctgaacggcctgctggcgacggtggtgaagcgtcaaaagtcggtgagcacgcgtttccagatgcactcccgcaaggtcaaggagcaggacgctgatggcctgctcgatgagcagctgccggacctggcggtggccctggcctccctggtcgccgaggccgcagcccgcgagcaggagcagcgggctgctgctggcggcggtggcggcctgcgtcggaatccgagctgctcccgcagcacgggctccgacgatggcggcggtcgtcgcagccccactgcgcgctcccgttcgccctcctgctcccccagccgttcgccttcgccgggccgccgcctggcgcaacgccgcactcagagcctgcggaactcgtcctcgggcatggacgtgccgtccaacggcaacgacgccgacgacacgggcgcagtggcggcgtcctccagcacgctggcgccttccgcgagctttcccgcacccggtgcgtgcctgccggggacggctcggccttcctcgacacggttcggtcccactccgcctacttccggccctccacccgcaagcacgaacagcgcatcgggcgctcccgccgcttcggcgccacagccccctgctgatctgtcgagcttccaacagactcgccgccagcgcctccagcaacagctccagcgcccgcagctgagcactggtgggggtggtcccggcaacgcggacggcctggccccacgcagcagctcgtccttcacccgcgtgcccgccagccgtctggtgatgggcggcgtgagccagtcgctggacggtggcgtgtgggcctcggctgagggcgctgccgctgctgtcgtggccctgtccggcgaggtccgtccgtactcgtcgccgctccaggcgccctggacccagggcgacggcctgcaactgccccgcctcacgtccgctggcggtgccgcatcgcaccggacgcagccggcaggcggtgtggctggcgtgtcgggtgccgtcgtggacgctcgggctgctgccggcggtggcgctgcgtccccggcctggagcgccccaggcggcgctgcccccaccggcgagagctccggctcgggcgtctgcggcgcgatgagcccacgcctgcgcagctccggcagccgtagccgcttccagtcgccgtccagcagccgtcgcctgatcccgactggcgctgccaccggggtgagcggtggtggcggctccaacggtgcgccccctagctcgccgggtggctccgtgcccctgactcgcggccagagctggtgggccaagagcacagtggctcgggagctggtcttcctgaagtagggatcc

SEQ ID NO: 82 - DNA, 1365 bp, artificial sequence
  codon optimized
ggtgagctctcctcctccgcagcagccgagccagccactaatacactgctgaacggcctgctggcgacggtggtgaagcgtcaaaagtcggtgagcacgcgtttccagatgcactcccgcaaggtcaaggagcaggacgctgatggcctgctcgatgagcagctgccggacctggcggtggccctggcctccctggtcgccgaggccgcagcccgcgagcaggagcagcgggctgctgctggcggcggtggcggcctgcgtcggaatccgagctgctcccgcagcacgggctccgacgatggcggcggtcgtcgcagccccactgcgcgctcccgttcgccctcctgctcccccagccgttcgccttcgccgggccgccgcctggcgcaacgccgcactcagagcctgcggaactcgtcctcgggcatggacgtgccgtccaacggcaacgacgccgacgacacgggcgcagtggcggcgtcctccagcacgctggcgccttccgcgagctttcccgcacccggtgcgtgcctgccggggacggctcggccttcctcgacacggttcggtcccactccgcctacttccggccctccacccgcaagcacgaacagcgcatcgggcgctcccgccgcttcggcgccacagccccctgctgatctgtcgagcttccaacagactcgccgccagcgcctccagcaacagctccagcgcccgcagctgagcactggtgggggtggtcccggcaacgcggacggcctggccccacgcagcagctcgtccttcacccgcgtgcccgccagccgtctggtgatgggcggcgtgagccagtcgctggacggtggcgtgtgggcctcggctgagggcgctgccgctgctgtcgtggccctgtccggcgaggtccgtccgtactcgtcgccgctccaggcgccctggacccagggcgacggcctgcaactgccccgcctcacgtccgctggcggtgccgcatcgcaccggacgcagccggcaggcggtgtggctggcgtgtcgggtgccgtcgtggacgctcgggctgctgccggcggtggcgctgcgtccccggcctggagcgccccaggcggcgctgcccccaccggcgagagctccggctcgggcgtctgcggcgcgatgagcccacgcctgcgcagctccggcagccgtagccgcttccagtcgccgtccagcagccgtcgcctgatcccgactggcgctgccaccggggtgagcggtggtggcggctccaacggtgcgccccctagctcgccgggtggctccgtgcccctgactcgcggccagagctggtgggccaagagcacagtggctcgggagctggtcttcctgaag

SEQ ID NO: 83 - DNA, 1365 bp, Chlamydomonas reinhardtii
ggcgagttgagttcctccgcggcagccgagcccgccaccaacacgctgctgaacgggctgctggcgacagtggtgaagcggcagaagtctgtgtctacgcggtttcaaatgcactcccgcaaggtcaaggaacaggacgctgacgggctcctggacgaacagctgcccgacctggcggtggcgctggcgtcgctggtggcggaggcggcggcgcgggagcaggagcagcgggcagcggccggcggcggcggcggcctccgccggaacccctcttgctctcgctcaacgggaagcgacgacggcggcggccggcggtcgccgactgcccgaagtcgcagccccagctgcagccccagccgcagccctagccctggccggcggctcgctcaaaggaggacccagagcctacgcaacagcagcagcggcatggacgttccaagcaacggcaacgacgctgatgacacgggcgctgtcgccgcctcctcctcgacgctggcgccttcagcatcctttccggctcccggcgcctgcctgcctggcacagcacgccccagctccacccgcttcggccccacgcctccgacatcgggcccgccaccagcctcaaccaacagcgccagcggggcgccggcggcgtcggcgccccagccaccagctgacctgtccagcttccagcagacgcggcggcagcgcctgcaacagcagctccagcggccgcagctcagcaccggcggtggtggcccgggcaacgctgacggccttgcgccccgctcgtcctcctccttcacgcgcgtgcccgcctcccgcctggtgatgggcggcgtctcgcagtcgttggatggcggcgtctgggcgtcggcggagggcgcggcggccgctgtggtggcgctgtcaggcgaggtacggccgtactcgtcgcccttgcaggcgccgtggacccagggagacggactccagctgccgcgcctgaccagcgctggcggggcggcaagccaccgcacgcagcccgcgggcggcgttgccggggtgtcaggcgctgtggtcgatgcgagggcagcagcaggcggcggagccgcttcgcctgcgtggtcagcgcccggcggtgctgctccgacgggggagagcagcggcagcggtgtgtgtggtgccatgtcgccgcgattgcgcagcagcggcagccgcagtcgcttccaatcgccgtcatccagccgccgcctgataccgacgggtgccgctactggcgtttctggtggcggtggcagcaacggcgcgccaccatcatcgcctggcggctccgtgcctctcacacggggccagtcctggtgggctaagagcaccgtggcacgagagctggtgttcctcaaa

SEQ ID NO: 84 - protein, 455 aa, Chlamydomonas reinhardtii
GELSSSAAAEPATNTLLNGLLATVVKRQKSVSTRFQMHSRKVKEQDADGLLDEQLPDLAVALASLVAEAAAREQEQRAAAGGGGGLRRNPSCSRSTGSDDGGGRRSPTARSRSPSCSPSRSPSPGRRLAQRRTQSLRNSSSGMDVPSNGNDADDTGAVAASSSTLAPSASFPAPGACLPGTARPSSTRFGPTPPTSGPPPASTNSASGAPAASAPQPPADLSSFQQTRRQRLQQQLQRPQLSTGGGGPGNADGLAPRSSSSFTRVPASRLVMGGVSQSLDGGVWASAEGAAAAVVALSGEVRPYSSPLQAPWTQGDGLQLPRLTSAGGAASHRTQPAGGVAGVSGAVVDARAAAGGGAASPAWSAPGGAAPTGESSGSGVCGAMSPRLRSSGSRSRFQSPSSSRRLIPTGAATGVSGGGGSNGAPPSSPGGSVPLTRGQSWWAKSTVARELVFLK

SEQ ID NO: 85 - DNA, 2046 bp, Chlamydomonas reinhardtii
atgcagcagaatgacctaatcttggcgccctggcttcctgatgagcagggacaagccgagctggagctgcagcagcaacagcagcagcagcagcagcttacgtggcaatggcagcacgccgctgacatggacccaccgcagcaagagtctcagcagcaacacacacaacagctgcagctgcagcagcagtattattacatcaagcctgaccccgcagcggccggcgacgcagccacaggcttccctgctgctcctggcgtggctgcgcccggcccctactacaaccagcaacagcagcagcagcaacaggcccacgccgctgcgggcaccttgttcggtcgccagggtgtccccgccgcggccatgtcgtctcaggcctactggcaaaaccagcagcggcaggagcagcagcaaggcggcggcatgttcgcttcggcgcccgcgacgggatacggcacgttcgcgcaacagcagcagcaacagcagtacacctcgccacttcttggcactgcaactactgcagctagcggtggttggggcgggggcggcggaggtggcggcggcggtttcaggatgggcagtagcagcggcaacctcatgcgagccgcgtcgctgaccacgcctgtgtctgccgccgccgccgccgccgccgccgcggcagtcgtgccacatcagcgcggaggctggatggcggcgagcggcggcggcggcaacggcagcgatagtagcggcatggcgctagcggcggctgcagcagcaggcgatggcgctgtgcggatgctgcagccgccaccgttgaacgtagcaggaagtattgccggcggcggcggcgaggttgattacctcctgcaaatgcaacagcagcaccagctgcagctgcagctgctgcaacagcaccagcagcagccggagcagcggctgtactgctcctggacagacggaggcgccgaggtgtgggagacagtgttcggtgagccagctccgcaggacacctttggtgccagcggcagtagtgggcgtgcatatcagcaagacctaggggcccacgcttctggcgcgcacccggcgggcccgcctccgccgccgcagccgctgccgccgccgcagccgcaacagccagccgccgccgccgccgccatgatgcagccgtacaacaaatccatggcggcgatgtctgcttcgcccacgccgccgccgccatctgagctgctgcagcagctgccgatggattgcggccgcagcagcggcccaaacaccataggcggcggccatggcagtagtcgcgcctggtcaaccggcggcagcggctatgatgtcgcggcggaggcggctgcagctactgatggcgctgctgctgctgctggtagtgatgttggtgagagcggattcagcggcaacgcagcagatggcattggcaagagcggcggtaccaccacggtgaccatgatgttgtgcatgcaacaactgcagcagccggcggcggcggcggcggcgggttgcatcaacaacaacctcgccgacagttggatgaagatgcagcagggcggcggcggcggcagcgatggcggcggcagcggctcgcaacagcattactatagcggcgtcggcgttaagaatgccgttgccggtgcgccgctgcgctcccatctgctgtttgggaatgatggcggcggcgtggacggcctcgcgaccaagcttgggttgccgccagctccgccgccgccggtgaccgcgacttgggactaccgccgcgctagccacgccgccgctggcggcggcgggaacaccagcagcagcagttacggaaccggcggcggttatttgcatgcgttgactcatcagcagtgcggcgctggtggcggcggcagcggcgccgccagcagcggtgtgtggctgctgcagcagcagcagtcgccgcccacagctcctggcacgcccccaacatgcggcatgacaacgtccttctcagcaacaggtggcggtggctcggacactgcaggccgccagcagggcaagcccccattcaagcaatcaaacatcatttga

SEQ ID NO: 86 - protein, 681 aa, Chlamydomonas reinhardtii
MQQNDLILAPWLPDEQGQAELELQQQQQQQQQLTWQWQHAADMDPPQQESQQQHTQQLQLQQQYYYIKPDPAAAGDAATGFPAAPGVAAPGPYYNQQQQQQQQAHAAAGTLFGRQGVPAAAMSSQAYWQNQQRQEQQQGGGMFASAPATGYGTFAQQQQQQQYTSPLLGTATTAASGGWGGGGGGGGGGFRMGSSSGNLMRAASLTTPVSAAAAAAAAAAVVPHQRGGWMAASGGGGNGSDSSGMALAAAAAAGDGAVRMLQPPPLNVAGSIAGGGGEVDYLLQMQQQHQLQLQLLQQHQQQPEQRLYCSWTDGGAEVWETVFGEPAPQDTFGASGSSGRAYQQDLGAHASGAHPAGPPPPPQPLPPPQPQQPAAAAAAMMQPYNKSMAAMSASPTPPPPSELLQQLPMDCGRSSGPNTIGGGHGSSRAWSTGGSGYDVAAEAAAATDGAAAAAGSDVGESGFSGNAADGIGKSGGTTTVTMMLCMQQLQQPAAAAAAGCINNNLADSWMKMQQGGGGGSDGGGSGSQQHYYSGVGVKNAVAGAPLRSHLLFGNDGGGVDGLATKLGLPPAPPPPVTATWDYRRASHAAAGGGGNTSSSSYGTGGGYLHALTHQQCGAGGGGSGAASSGVWLLQQQQSPPTAPGTPPTCGMTTSFSATGGGGSDTAGRQQGKPPFKQSNII

SEQ ID NO: 87 - DNA, 2061 bp, artificial sequence
  codon optimized
catatgctcgagcaacaaaatgatctgattctggcgccgtggctgcctgatgagcaaggccaggccgagctggagctccagcagcagcaacagcagcagcaacaactgacgtggcagtggcagcacgctgctgatatggacccgcctcaacaggagagccaacagcagcacacgcagcagctccagctgcaacagcagtactactacatcaagccggaccccgccgcagctggcgacgccgccaccggcttcccagcggcgcctggcgtcgcggcgcctgggccctactacaaccagcaacaacaacagcagcaacaggcccacgctgccgcaggcacactgttcgggcgccagggcgtgcctgcggcagccatgtcgtcccaggcttactggcagaaccaacagcgccaggagcaacagcagggtggtggcatgttcgcctcggctcccgcgacgggttacggcacgttcgctcaacagcaacaacagcaacagtacacctccccgctgctgggcaccgccacgacggccgcttcgggcggctggggcggcggcggcggcgggggcggtggcggcttccggatgggctcgtcgtcgggcaacctgatgcgcgctgcgtccctgaccacccccgtctccgccgctgcggcagcggcggctgcggcggctgtggtgccccaccagcgcggtggctggatggccgcgtccggtggcggcggcaacggtagcgacagctcggggatggccctggcggctgccgctgctgctggcgatggcgctgtgcgtatgctccagccccctccgctgaacgtcgctgggtcgattgcggggggggggggcgaggtggattacctcctccagatgcagcagcagcaccagctccagctgcaactgctccagcagcaccagcagcagccagagcagcgcctctactgctcgtggaccgacggtggcgccgaggtgtgggagacggtgttcggcgagcccgctccccaggacacttttggcgcctccggttcgtcgggtcgtgcctaccagcaggacctgggcgcgcacgcctcgggcgctcacccggctggccctccgccacccccacagccactgcctccgccgcagccgcaacagcccgctgcggcagcggcagcgatgatgcagccctataacaagagcatggcggccatgtccgccagccccacgcctcccccgccctcggagctgctgcaacagctgcctatggactgcggccgttcgagcggccctaacaccatcggcggtggccacggctcctcccgcgcctggagcaccggcggcagcggctacgacgtggctgcggaggccgcagctgcgaccgatggcgcagccgcggcggctggttccgacgtgggcgagtcggggttctcgggcaacgcagcggacggcatcggcaagtccggcggcaccaccaccgtgacgatgatgctgtgtatgcagcagctccagcaacccgcggctgccgctgccgcagggtgcatcaacaataacctggctgatagctggatgaagatgcagcagggcggtggtggcggcagcgacggcggtggctcgggctcgcagcagcactactactcgggcgtgggggtgaagaacgccgtggcaggcgccccactgcgcagccacctgctcttcggcaacgacgggggtggcgtggacgggctcgcgaccaagctcggcctgcccccagctcccccgcctcccgtgactgcgacgtgggactatcgccgcgcgagccacgctgcggctggggggggcggcaacacgagcagcagctcctatggcactgggggtggctacctgcacgctctgacccaccagcagtgcggcgctggcggcggtggttccggcgcagcgtccagcggcgtgtggctgctgcaacaacagcagtccccgcccacggcgcctggcacccctccgacctgcggcatgaccacgtcgttcagcgccactggcggtggtggctccgacaccgctggccgccagcagggcaagcccccgttcaagcagtccaacatcatctagggatcc

SEQ ID NO: 88 - DNA, 2040 bp, artificial sequence
  codon optimized
caacaaaatgatctgattctggcgccgtggctgcctgatgagcaaggccaggccgagctggagctccagcagcagcaacagcagcagcaacaactgacgtggcagtggcagcacgctgctgatatggacccgcctcaacaggagagccaacagcagcacacgcagcagctccagctgcaacagcagtactactacatcaagccggaccccgccgcagctggcgacgccgccaccggcttcccagcggcgcctggcgtcgcggcgcctgggccctactacaaccagcaacaacaacagcagcaacaggcccacgctgccgcaggcacactgttcgggcgccagggcgtgcctgcggcagccatgtcgtcccaggcttactggcagaaccaacagcgccaggagcaacagcagggtggtggcatgttcgcctcggctcccgcgacgggttacggcacgttcgctcaacagcaacaacagcaacagtacacctccccgctgctgggcaccgccacgacggccgcttcgggcggctggggcggcggcggcggcgggggcggtggcggcttccggatgggctcgtcgtcgggcaacctgatgcgcgctgcgtccctgaccacccccgtctccgccgctgcggcagcggcggctgcggcggctgtggtgccccaccagcgcggtggctggatggccgcgtccggtggcggcggcaacggtagcgacagctcggggatggccctggcggctgccgctgctgctggcgatggcgctgtgcgtatgctccagccccctccgctgaacgtcgctgggtcgattgcggggggggggggcgaggtggattacctcctccagatgcagcagcagcaccagctccagctgcaactgctccagcagcaccagcagcagccagagcagcgcctctactgctcgtggaccgacggtggcgccgaggtgtgggagacggtgttcggcgagcccgctccccaggacacttttggcgcctccggttcgtcgggtcgtgcctaccagcaggacctgggcgcgcacgcctcgggcgctcacccggctggccctccgccacccccacagccactgcctccgccgcagccgcaacagcccgctgcggcagcggcagcgatgatgcagccctataacaagagcatggcggccatgtccgccagccccacgcctcccccgccctcggagctgctgcaacagctgcctatggactgcggccgttcgagcggccctaacaccatcggcggtggccacggctcctcccgcgcctggagcaccggcggcagcggctacgacgtggctgcggaggccgcagctgcgaccgatggcgcagccgcggcggctggttccgacgtgggcgagtcggggttctcgggcaacgcagcggacggcatcggcaagtccggcggcaccaccaccgtgacgatgatgctgtgtatgcagcagctccagcaacccgcggctgccgctgccgcagggtgcatcaacaataacctggctgatagctggatgaagatgcagcagggcggtggtggcggcagcgacggcggtggctcgggctcgcagcagcactactactcgggcgtgggggtgaagaacgccgtggcaggcgccccactgcgcagccacctgctcttcggcaacgacgggggtggcgtggacgggctcgcgaccaagctcggcctgcccccagctcccccgcctcccgtgactgcgacgtgggactatcgccgcgcgagccacgctgcggctggggggggcggcaacacgagcagcagctcctatggcactgggggtggctacctgcacgctctgacccaccagcagtgcggcgctggcggcggtggttccggcgcagcgtccagcggcgtgtggctgctgcaacaacagcagtccccgcccacggcgcctggcacccctccgacctgcggcatgaccacgtcgttcagcgccactggcggtggtggctccgacaccgctggccgccagcagggcaagcccccgttcaagcagtccaacatcatc

SEQ ID NO: 89 - DNA, 2040 bp, Chlamydomonas reinhardtii
cagcagaatgacctaatcttggcgccctggcttcctgatgagcagggacaagccgagctggagctgcagcagcaacagcagcagcagcagcagcttacgtggcaatggcagcacgccgctgacatggacccaccgcagcaagagtctcagcagcaacacacacaacagctgcagctgcagcagcagtattattacatcaagcctgaccccgcagcggccggcgacgcagccacaggcttccctgctgctcctggcgtggctgcgcccggcccctactacaaccagcaacagcagcagcagcaacaggcccacgccgctgcgggcaccttgttcggtcgccagggtgtccccgccgcggccatgtcgtctcaggcctactggcaaaaccagcagcggcaggagcagcagcaaggcggcggcatgttcgcttcggcgcccgcgacgggatacggcacgttcgcgcaacagcagcagcaacagcagtacacctcgccacttcttggcactgcaactactgcagctagcggtggttggggcgggggcggcggaggtggcggcggcggtttcaggatgggcagtagcagcggcaacctcatgcgagccgcgtcgctgaccacgcctgtgtctgccgccgccgccgccgccgccgccgcggcagtcgtgccacatcagcgcggaggctggatggcggcgagcggcggcggcggcaacggcagcgatagtagcggcatggcgctagcggcggctgcagcagcaggcgatggcgctgtgcggatgctgcagccgccaccgttgaacgtagcaggaagtattgccggcggcggcggcgaggttgattacctcctgcaaatgcaacagcagcaccagctgcagctgcagctgctgcaacagcaccagcagcagccggagcagcggctgtactgctcctggacagacggaggcgccgaggtgtgggagacagtgttcggtgagccagctccgcaggacacctttggtgccagcggcagtagtgggcgtgcatatcagcaagacctaggggcccacgcttctggcgcgcacccggcgggcccgcctccgccgccgcagccgctgccgccgccgcagccgcaacagccagccgccgccgccgccgccatgatgcagccgtacaacaaatccatggcggcgatgtctgcttcgcccacgccgccgccgccatctgagctgctgcagcagctgccgatggattgcggccgcagcagcggcccaaacaccataggcggcggccatggcagtagtcgcgcctggtcaaccggcggcagcggctatgatgtcgcggcggaggcggctgcagctactgatggcgctgctgctgctgctggtagtgatgttggtgagagcggattcagcggcaacgcagcagatggcattggcaagagcggcggtaccaccacggtgaccatgatgttgtgcatgcaacaactgcagcagccggcggcggcggcggcggcgggttgcatcaacaacaacctcgccgacagttggatgaagatgcagcagggcggcggcggcggcagcgatggcggcggcagcggctcgcaacagcattactatagcggcgtcggcgttaagaatgccgttgccggtgcgccgctgcgctcccatctgctgtttgggaatgatggcggcggcgtggacggcctcgcgaccaagcttgggttgccgccagctccgccgccgccggtgaccgcgacttgggactaccgccgcgctagccacgccgccgctggcggcggcgggaacaccagcagcagcagttacggaaccggcggcggttatttgcatgcgttgactcatcagcagtgcggcgctggtggcggcggcagcggcgccgccagcagcggtgtgtggctgctgcagcagcagcagtcgccgcccacagctcctggcacgcccccaacatgcggcatgacaacgtccttctcagcaacaggtggcggtggctcggacactgcaggccgccagcagggcaagcccccattcaagcaatcaaacatcatt

SEQ ID NO: 90 - protein, 680 aa, Chlamydomonas reinhardtii
QQNDLILAPWLPDEQGQAELELQQQQQQQQQLTWQWQHAADMDPPQQESQQQHTQQLQLQQQYYYIKPDPAAAGDAATGFPAAPGVAAPGPYYNQQQQQQQQAHAAAGTLFGRQGVPAAAMSSQAYWQNQQRQEQQQGGGMFASAPATGYGTFAQQQQQQQYTSPLLGTATTAASGGWGGGGGGGGGGFRMGSSSGNLMRAASLTTPVSAAAAAAAAAAVVPHQRGGWMAASGGGGNGSDSSGMALAAAAAAGDGAVRMLQPPPLNVAGSIAGGGGEVDYLLQMQQQHQLQLQLLQQHQQQPEQRLYCSWTDGGAEVWETVFGEPAPQDTFGASGSSGRAYQQDLGAHASGAHPAGPPPPPQPLPPPQPQQPAAAAAAMMQPYNKSMAAMSASPTPPPPSELLQQLPMDCGRSSGPNTIGGGHGSSRAWSTGGSGYDVAAEAAAATDGAAAAAGSDVGESGFSGNAADGIGKSGGTTTVTMMLCMQQLQQPAAAAAAGCINNNLADSWMKMQQGGGGGSDGGGSGSQQHYYSGVGVKNAVAGAPLRSHLLFGNDGGGVDGLATKLGLPPAPPPPVTATWDYRRASHAAAGGGGNTSSSSYGTGGGYLHALTHQQCGAGGGGSGAASSGVWLLQQQQSPPTAPGTPPTCGMTTSFSATGGGGSDTAGRQQGKPPFKQSNII

SEQ ID NO: 91 - DNA, 1875 bp, Chlamydomonas reinhardtii
atgagtgcggtcgaggttcaccataccgagaaacgctcctttaaggagcttatggtcggaacctacgacctgaaatatctctgcctgcctgcttttccatactttcggaaggggaaaggcgtacccccgccgcgcttcttcggcaaggatgattttctgggactgttcgtggccctggtcatgggcctgcagcacgcgctggccatggtgggaggcctcatcacgccgccgctgctagtgtcaaccctggccttcggcaaccggcctgggaccacaactccctatgcccagggcgatccagctgaaattcagcgctacctggtccaggcggcactcatcgtgtgcggtatcatgacctttttccaagtcctgggcgtgcgcatttacaaaaccaagtttcagtacggagcgggtgtgctctcttgtatgggcatctcctttacaaccgtgcccattgcgacctccgtcattggacagctcatgaaggagcagggacgaacttttgaggaggcgtacggcaactttctcggcaccatcgccatgtgcggggtaatccccgtaatcctttcattctttcccattcgtgtcatcaaaaagatcttcccgccccttgtgtgcggcattgtcattatgatgattggcgtgcatctgatcggttctgggtttaagaactggggcggcggcgccttctgtgccgataactggcagcacccgcccgcaaccagagcgtgcttcctgcccgccaagtaccccaacggcaccagttacttgcagctgaaccaatgctgggtgggccccggcgtcatgtgcggcgacaagaccaagaccgaggtattccttcctttcggctctcaggagtacctcggcctgggctttttggtcttcatcaccatcatcttcttggagatcttcggcagccccttcatgcgcaacgcctctgtcattctggcgctgctgttcggctacctcatcgccgctgtcaccacctaccagggcaagaagtacgtgatcaccaccaagattgaccaggcgcccggcatcaccttcctgtggaccaccaccttcccgctaggtttctatccgcccgccattatcccgctgatgattgtgtttatcatcacatccattgagaccgtgggcgacacttcagcaactatggaggcatcacgtatggctgttgacaccgaggacggcacgcggcgcattaagggcgcgctcctgaacgacggcatcagcggcatcttctccgccctggccacctcgctgcccctgaccacgttcgctcagaacaacggcgtcatagctttgaccaacgtggccgctcgtcaggccggctttgctgctgcgttctggctgttcctgctgggcattctaggcaaggtgggcgcctggatcaccaccatcccggagtgtgtgctgggcggcatgaccactttcctgttcgccaacgtcattgcctcgggcattaagattatcatcaacggcgatcccctcacgcgccgctcgcgcttcatcctggcgtgctcattggcgctggcgtttggcgtggagctggtcccgcagtgggccacgctcaacctgtggcccgtcacgcccggcatgtcgcccggcctgcgcggcctgcgtgacgccatcatcctggtcatctccacctccttcaccctgggcgccgtggtggcgctcatcctcaacctcatcataccgctggacaagaccgatcctacagtcacaagatgctctccaggtgctagctcggtcagcactgagaatgacgggaaggatgcttcgttccactcagacgcggcacaggccagcagtgctccaccgccggtggtgaccatcacgcagagacacagcagcaatggcttcgcgtag

SEQ ID NO: 92 - protein, 624 aa, Chlamydomonas reinhardtii
MSAVEVHHTEKRSFKELMVGTYDLKYLCLPAFPYFRKGKGVPPPRFFGKDDFLGLFVALVMGLQHALAMVGGLITPPLLVSTLAFGNRPGTTTPYAQGDPAEIQRYLVQAALIVCGIMTFFQVLGVRIYKTKFQYGAGVLSCMGISFTTVPIATSVIGQLMKEQGRTFEEAYGNFLGTIAMCGVIPVILSFFPIRVIKKIFPPLVCGIVIMMIGVHLIGSGFKNWGGGAFCADNWQHPPATRACFLPAKYPNGTSYLQLNQCWVGPGVMCGDKTKTEVFLPFGSQEYLGLGFLVFITIIFLEIFGSPFMRNASVILALLFGYLIAAVTTYQGKKYVITTKIDQAPGITFLWTTTFPLGFYPPAIIPLMIVFIITSIETVGDTSATMEASRMAVDTEDGTRRIKGALLNDGISGIFSALATSLPLTTFAQNNGVIALTNVAARQAGFAAAFWLFLLGILGKVGAWITTIPECVLGGMTTFLFANVIASGIKIIINGDPLTRRSRFILACSLALAFGVELVPQWATLNLWPVTPGMSPGLRGLRDAIILVISTSFTLGAVVALILNLIIPLDKTDPTVTRCSPGASSVSTENDGKDASFHSDAAQASSAPPPVVTITQRHSSNGFA

SEQ ID NO: 93 - DNA, 1890 bp, artificial sequence
  codon optimized
catatgctcgagtcggcagtcgaggtgcatcatacagagaagcgttcgtttaaggagctgatggtgggcacctacgacctgaagtacctgtgcctgcccgctttcccgtacttccgcaagggcaagggggtcccgcctcctcgcttcttcggcaaggatgacttcctcggcctgttcgtggcgctcgtgatgggcctccagcacgccctggcgatggtcggcgggctgattacgcctcccctgctcgtgagcaccctggcgttcggtaaccgccctggcaccacgacaccgtacgcgcagggggaccccgccgagatccagcgctacctggtgcaggctgccctgatcgtctgcggcatcatgaccttctttcaggtgctgggggtgcgcatctacaagaccaagttccagtacggtgccggggtgctgtcgtgcatgggcatctcgttcacgacggtgcccatcgcaacatccgtgatcggccaactgatgaaggagcagggccgcacgtttgaggaggcgtacggcaacttcctgggcaccatcgctatgtgcggcgtgatcccagtgatcctgtcgtttttccccatccgcgtcattaagaagattttccctcctctggtgtgcggcatcgtgatcatgatgattggcgtgcacctgattggttcggggttcaagaactggggcggcggtgcgttctgcgccgataactggcagcaccctcctgctactcgcgcctgcttcctgcctgctaagtaccctaacggcacctcctacctccagctgaaccagtgctgggtgggccctggggtgatgtgcggggataagaccaagaccgaggtcttcctccccttcggctcccaggagtacctgggcctcggcttcctggtgtttatcacgatcatcttcctggagattttcggcagcccgttcatgcggaacgccagcgtgatcctggccctgctgttcggctacctgatcgctgccgtgacgacgtatcagggtaagaagtatgtcatcacgacgaagattgaccaggcccctggcatcactttcctgtggacgacgaccttccccctgggcttttatcctccagccattatcccgctgatgattgtgttcatcatcacctccatcgagactgtgggcgacacgtcggccacgatggaggcgtcccgcatggcagtggacaccgaggatggcactcggcgcatcaagggcgctctgctgaacgacgggatctccggcatcttcagcgcactggccacgtccctgcccctgaccaccttcgcccagaacaacggggtcatcgccctgaccaacgtcgccgcacgtcaggctggcttcgctgcggccttctggctgttcctgctgggcattctcggcaaggtgggggcctggatcaccaccatccccgagtgcgtgctcggcggcatgacgacattcctgtttgcgaacgtcatcgccagcggcatcaagatcatcatcaacggcgaccccctgacccgccgcagccggttcattctggcctgctcgctggcgctggccttcggtgtcgagctcgtgccgcagtgggctacgctgaatctctggcccgtgacccccggtatgagccctggtctgcgcggcctgcgcgacgccatcatcctggtgatcagcaccagctttacactgggggcggtggtggcgctgatcctgaacctcatcatcccgctggacaagacggacccaacggtgacccgttgctccccaggcgcatcctcggtgagcaccgagaacgacggcaaggacgcatcgttccactccgacgcggcccaggccagctcggccccccctcccgtggtgaccatcacccagcgccactccagcaatggcttcgcgtgaggatcc

SEQ ID NO: 94 - DNA, 1869 bp, artificial sequence
  codon optimized
tcggcagtcgaggtgcatcatacagagaagcgttcgtttaaggagctgatggtgggcacctacgacctgaagtacctgtgcctgcccgctttcccgtacttccgcaagggcaagggggtcccgcctcctcgcttcttcggcaaggatgacttcctcggcctgttcgtggcgctcgtgatgggcctccagcacgccctggcgatggtcggcgggctgattacgcctcccctgctcgtgagcaccctggcgttcggtaaccgccctggcaccacgacaccgtacgcgcagggggaccccgccgagatccagcgctacctggtgcaggctgccctgatcgtctgcggcatcatgaccttctttcaggtgctgggggtgcgcatctacaagaccaagttccagtacggtgccggggtgctgtcgtgcatgggcatctcgttcacgacggtgcccatcgcaacatccgtgatcggccaactgatgaaggagcagggccgcacgtttgaggaggcgtacggcaacttcctgggcaccatcgctatgtgcggcgtgatcccagtgatcctgtcgtttttccccatccgcgtcattaagaagattttccctcctctggtgtgcggcatcgtgatcatgatgattggcgtgcacctgattggttcggggttcaagaactggggcggcggtgcgttctgcgccgataactggcagcaccctcctgctactcgcgcctgcttcctgcctgctaagtaccctaacggcacctcctacctccagctgaaccagtgctgggtgggccctggggtgatgtgcggggataagaccaagaccgaggtcttcctccccttcggctcccaggagtacctgggcctcggcttcctggtgtttatcacgatcatcttcctggagattttcggcagcccgttcatgcggaacgccagcgtgatcctggccctgctgttcggctacctgatcgctgccgtgacgacgtatcagggtaagaagtatgtcatcacgacgaagattgaccaggcccctggcatcactttcctgtggacgacgaccttccccctgggcttttatcctccagccattatcccgctgatgattgtgttcatcatcacctccatcgagactgtgggcgacacgtcggccacgatggaggcgtcccgcatggcagtggacaccgaggatggcactcggcgcatcaagggcgctctgctgaacgacgggatctccggcatcttcagcgcactggccacgtccctgcccctgaccaccttcgcccagaacaacggggtcatcgccctgaccaacgtcgccgcacgtcaggctggcttcgctgcggccttctggctgttcctgctgggcattctcggcaaggtgggggcctggatcaccaccatccccgagtgcgtgctcggcggcatgacgacattcctgtttgcgaacgtcatcgccagcggcatcaagatcatcatcaacggcgaccccctgacccgccgcagccggttcattctggcctgctcgctggcgctggccttcggtgtcgagctcgtgccgcagtgggctacgctgaatctctggcccgtgacccccggtatgagccctggtctgcgcggcctgcgcgacgccatcatcctggtgatcagcaccagctttacactgggggcggtggtggcgctgatcctgaacctcatcatcccgctggacaagacggacccaacggtgacccgttgctccccaggcgcatcctcggtgagcaccgagaacgacggcaaggacgcatcgttccactccgacgcggcccaggccagctcggccccccctcccgtggtgaccatcacccagcgccactccagcaatggcttcgcg

SEQ ID NO: 95 - DNA, 1869 bp, Chlamydomonas reinhardtii
agtgcggtcgaggttcaccataccgagaaacgctcctttaaggagcttatggtcggaacctacgacctgaaatatctctgcctgcctgcttttccatactttcggaaggggaaaggcgtacccccgccgcgcttcttcggcaaggatgattttctgggactgttcgtggccctggtcatgggcctgcagcacgcgctggccatggtgggaggcctcatcacgccgccgctgctagtgtcaaccctggccttcggcaaccggcctgggaccacaactccctatgcccagggcgatccagctgaaattcagcgctacctggtccaggcggcactcatcgtgtgcggtatcatgacctttttccaagtcctgggcgtgcgcatttacaaaaccaagtttcagtacggagcgggtgtgctctcttgtatgggcatctcctttacaaccgtgcccattgcgacctccgtcattggacagctcatgaaggagcagggacgaacttttgaggaggcgtacggcaactttctcggcaccatcgccatgtgcggggtaatccccgtaatcctttcattctttcccattcgtgtcatcaaaaagatcttcccgccccttgtgtgcggcattgtcattatgatgattggcgtgcatctgatcggttctgggtttaagaactggggcggcggcgccttctgtgccgataactggcagcacccgcccgcaaccagagcgtgcttcctgcccgccaagtaccccaacggcaccagttacttgcagctgaaccaatgctgggtgggccccggcgtcatgtgcggcgacaagaccaagaccgaggtattccttcctttcggctctcaggagtacctcggcctgggctttttggtcttcatcaccatcatcttcttggagatcttcggcagccccttcatgcgcaacgcctctgtcattctggcgctgctgttcggctacctcatcgccgctgtcaccacctaccagggcaagaagtacgtgatcaccaccaagattgaccaggcgcccggcatcaccttcctgtggaccaccaccttcccgctaggtttctatccgcccgccattatcccgctgatgattgtgtttatcatcacatccattgagaccgtgggcgacacttcagcaactatggaggcatcacgtatggctgttgacaccgaggacggcacgcggcgcattaagggcgcgctcctgaacgacggcatcagcggcatcttctccgccctggccacctcgctgcccctgaccacgttcgctcagaacaacggcgtcatagctttgaccaacgtggccgctcgtcaggccggctttgctgctgcgttctggctgttcctgctgggcattctaggcaaggtgggcgcctggatcaccaccatcccggagtgtgtgctgggcggcatgaccactttcctgttcgccaacgtcattgcctcgggcattaagattatcatcaacggcgatcccctcacgcgccgctcgcgcttcatcctggcgtgctcattggcgctggcgtttggcgtggagctggtcccgcagtgggccacgctcaacctgtggcccgtcacgcccggcatgtcgcccggcctgcgcggcctgcgtgacgccatcatcctggtcatctccacctccttcaccctgggcgccgtggtggcgctcatcctcaacctcatcataccgctggacaagaccgatcctacagtcacaagatgctctccaggtgctagctcggtcagcactgagaatgacgggaaggatgcttcgttccactcagacgcggcacaggccagcagtgctccaccgccggtggtgaccatcacgcagagacacagcagcaatggcttcgcg

SEQ ID NO: 96 - protein, 623 aa, Chlamydomonas reinhardtii
SAVEVHHTEKRSFKELMVGTYDLKYLCLPAFPYFRKGKGVPPPRFFGKDDFLGLFVALVMGLQHALAMVGGLITPPLLVSTLAFGNRPGTTTPYAQGDPAEIQRYLVQAALIVCGIMTFFQVLGVRIYKTKFQYGAGVLSCMGISFTTVPIATSVIGQLMKEQGRTFEEAYGNFLGTIAMCGVIPVILSFFPIRVIKKIFPPLVCGIVIMMIGVHLIGSGFKNWGGGAFCADNWQHPPATRACFLPAKYPNGTSYLQLNQCWVGPGVMCGDKTKTEVFLPFGSQEYLGLGFLVFITIIFLEIFGSPFMRNASVILALLFGYLIAAVTTYQGKKYVITTKIDQAPGITFLWTTTFPLGFYPPAIIPLMIVFIITSIETVGDTSATMEASRMAVDTEDGTRRIKGALLNDGISGIFSALATSLPLTTFAQNNGVIALTNVAARQAGFAAAFWLFLLGILGKVGAWITTIPECVLGGMTTFLFANVIASGIKIIINGDPLTRRSRFILACSLALAFGVELVPQWATLNLWPVTPGMSPGLRGLRDAIILVISTSFTLGAVVALILNLIIPLDKTDPTVTRCSPGASSVSTENDGKDASFHSDAAQASSAPPPVVTITQRHSSNGFA

SEQ ID NO: 97 - DNA, 804 bp, Chlamydomonas reinhardtii
atggaccccgctgctgaagccgcggctcaggctgccgctcaggctgcccaggccgctgctgctcagcaggctctcgcccagcaggctgccgctgcggccgctgccgctgcagctgcggctgccgccgcgccgcgtgccggctctggtggctccgctgctccgtcgtctgtccacctgccccctcgccctgcatccgcgccgggcatgggctcgaacaacgacgacgagatgctctccgccggcgaaggcgatgagtacggctacgaccaccagccctccgctcccatggcgccggccagctacggccccggccccacctacggtcacgccgggcctgcctttggcaccggcggtcctgccttccaccagccgcccagcgtcatgtcgcagctggacaccctgcgccaccggcagaacggccacggccacctgctggaggacgtccagcgcaccggccacggcacccagcaggcggtcgagagtctgcgcaacgggctgcagaacatgcactcgctgctggtcaacaccaacactgacgtggccaacctccgcaactctcaggctccgctgaacgccgccgtccacgagattcagctggcgctcggacgcctggagaacaagttcgacgccctgcgcgaccgcatgggcgacggcgccggcggctccgcgaagcgccagcgctcggaggaggacaagagcattgccggcctgaagactctcgggttctgcaccaagtgcgcccgtgaggacgtcagccgcaagtggaacacctgccgcgaccacaaccccaagtttgccgcgtag

SEQ ID NO: 98 - protein, 267 aa, Chlamydomonas reinhardtii
MDPAAEAAAQAAAQAAQAAAAQQALAQQAAAAAAAAAAAAAAAPRAGSGGSAAPSSVHLPPRPASAPGMGSNNDDEMLSAGEGDEYGYDHQPSAPMAPASYGPGPTYGHAGPAFGTGGPAFHQPPSVMSQLDTLRHRQNGHGHLLEDVQRTGHGTQQAVESLRNGLQNMHSLLVNTNTDVANLRNSQAPLNAAVHEIQLALGRLENKFDALRDRMGDGAGGSAKRQRSEEDKSIAGLKTLGFCTKCAREDVSRKWNTCRDHNPKFAA

SEQ ID NO: 99 - DNA, 825 bp, artificial sequence
  codon optimized
catatgctcgaggacccagcggcggaggcagcagctcaagcagcagcacaagcagcacaggccgctgccgcgcagcaggccctggcgcagcaggctgccgctgcggcagcggcagccgcagcagcagcggctgcggcgcctcgcgcaggctcgggcggctcggctgccccgagcagcgtgcacctgccgcctcgccccgccagcgctcccggcatgggctcgaacaacgacgacgagatgctgtcggcgggcgagggggacgagtacgggtacgaccaccagccctccgcaccgatggcccctgcgtcctatggcccaggccctacctacggccacgcgggtcccgcatttggcactggtggccctgccttccatcagccacccagcgtgatgagccagctggacactctgcggcaccgccagaacggccacggccacctgctggaggatgtgcagcgcacgggccacggcacccagcaggccgtggagagcctgcgcaacgggctgcaaaacatgcatagcctgctggtgaacaccaacaccgacgtggcgaatctgcgcaactcccaagccccgctgaacgcggccgtgcacgagatccaactggctctgggccggctggagaacaagttcgacgccctccgcgaccggatgggcgacggcgctggcgggtcggccaagcgtcagcgctccgaggaggacaagtcgatcgcgggcctgaagacgctgggcttttgcacgaagtgcgctcgggaggatgtgtcccggaagtggaatacctgccgcgaccacaacccaaagtttgcggccaccggttaaggatcc

SEQ ID NO: 100 - DNA, 798 bp, artificial sequence
  codon optimized
gacccagcggcggaggcagcagctcaagcagcagcacaagcagcacaggccgctgccgcgcagcaggccctggcgcagcaggctgccgctgcggcagcggcagccgcagcagcagcggctgcggcgcctcgcgcaggctcgggcggctcggctgccccgagcagcgtgcacctgccgcctcgccccgccagcgctcccggcatgggctcgaacaacgacgacgagatgctgtcggcgggcgagggggacgagtacgggtacgaccaccagccctccgcaccgatggcccctgcgtcctatggcccaggccctacctacggccacgcgggtcccgcatttggcactggtggccctgccttccatcagccacccagcgtgatgagccagctggacactctgcggcaccgccagaacggccacggccacctgctggaggatgtgcagcgcacgggccacggcacccagcaggccgtggagagcctgcgcaacgggctgcaaaacatgcatagcctgctggtgaacaccaacaccgacgtggcgaatctgcgcaactcccaagccccgctgaacgcggccgtgcacgagatccaactggctctgggccggctggagaacaagttcgacgccctccgcgaccggatgggcgacggcgctggcgggtcggccaagcgtcagcgctccgaggaggacaagtcgatcgcgggcctgaagacgctgggcttttgcacgaagtgcgctcgggaggatgtgtcccggaagtggaatacctgccgcgaccacaacccaaagtttgcggcc

SEQ ID NO: 101 - DNA, 798 bp, Chlamydomonas reinhardtii
gaccccgctgctgaagccgcggctcaggctgccgctcaggctgcccaggccgctgctgctcagcaggctctcgcccagcaggctgccgctgcggccgctgccgctgcagctgcggctgccgccgcgccgcgtgccggctctggtggctccgctgctccgtcgtctgtccacctgccccctcgccctgcatccgcgccgggcatgggctcgaacaacgacgacgagatgctctccgccggcgaaggcgatgagtacggctacgaccaccagccctccgctcccatggcgccggccagctacggccccggccccacctacggtcacgccgggcctgcctttggcaccggcggtcctgccttccaccagccgcccagcgtcatgtcgcagctggacaccctgcgccaccggcagaacggccacggccacctgctggaggacgtccagcgcaccggccacggcacccagcaggcggtcgagagtctgcgcaacgggctgcagaacatgcactcgctgctggtcaacaccaacactgacgtggccaacctccgcaactctcaggctccgctgaacgccgccgtccacgagattcagctggcgctcggacgcctggagaacaagttcgacgccctgcgcgaccgcatgggcgacggcgccggcggctccgcgaagcgccagcgctcggaggaggacaagagcattgccggcctgaagactctcgggttctgcaccaagtgcgcccgtgaggacgtcagccgcaagtggaacacctgccgcgaccacaaccccaagtttgccgcg

SEQ ID NO: 102 - protein, 266 aa, Chlamydomonas reinhardtii
DPAAEAAAQAAAQAAQAAAAQQALAQQAAAAAAAAAAAAAAAPRAGSGGSAAPSSVHLPPRPASAPGMGSNNDDEMLSAGEGDEYGYDHQPSAPMAPASYGPGPTYGHAGPAFGTGGPAFHQPPSVMSQLDTLRHRQNGHGHLLEDVQRTGHGTQQAVESLRNGLQNMHSLLVNTNTDVANLRNSQAPLNAAVHEIQLALGRLENKFDALRDRMGDGAGGSAKRQRSEEDKSIAGLKTLGFCTKCAREDVSRKWNTCRDHNPKFAA

SEQ ID NO: 103 - DNA, 1920 bp, Chlamydomonas reinhardtii
atgcgggtcagtcttcttctgtctgtggcgttcttgtctgtgctggcgctcccaagcgccacaaagctatcgtggtcacgactgcagacgtcagctgcgaaccagccctcgcctcggcgtggagctgcccttgtgcacgatgcgttcacagagcagttgttccttttcggtggcagcgatggcgagtcagtcgcgctgaacgatgtttgggtcttccagctgaacaaccagagttggcgacaactcaacgtgaccgggcctcgcccagctggccggcacaccttcgtgtacggcctgtacgtcaacagcagcagtagtgcctcgggcagcagcacggctgcgcgccccacacgtgtgctggttgtggccactgggcagggctccgccgtgttctccgacatatgggccctggatctggattcggtcacgtggcgacagctgccgcaagcgggggatccgcccaacacggtgtacggctccgcaggcggcatcgcgccctcggtcccgggcggcccgcacagcagccgcttctggctgtcgcacggcttctcctccaagcggcgctactcaaccactcactactatgacctacaggcggagaggtgggtccttgtgcacggcgccataaactcgtacgatgctacgggcccgcactcacgctgcatcgtgagcagcaccgtcatgagcgacgagcggatcgtcatgtacggaggctgcgcccagaacggaggcacgggcggcccctgccctgcgcgcgacgcctggacgtttgacgggtcaagctggaagcaggcctccacctgtcccacaccccgcacacgtggcgtcatggtgcccctcacgtcccctctcctgtcgctgcccacatcggctgacatggccgccgcggcggctcaggaggcgatgatggggccgggccgaacgggcagtggtggcataatggggcccgacagcctgaaccaaaccgccgggctgcgagtgtacggcgggcgttacgtgcttctgtacggcggttacgagcgcgacaagcagaccatctccgtgtcgtcggcaccagacgaccagctcccggtgctggatcttgacaatggcgagtggctgcttctgcgcgcgtcgggggaggtcccggcattcaggggccagcccgcggtcgcacacgacgtggcgaccgggcgtgtgtgggtgtttggcggccagctccgcggcagtggcgccctcagcaacgacctgtacgagctgcagggcgacccggaggccacgccgccctacccggatggctcatgtggctccaccttcctatacccgcatctgcacggcatatttatgggccttgcgtggggcatactgctacaggcaggctggtttatcgcgcgctacttcaagcggagcacgacttggttcaatctgcatcgtgcctgtcagatttccgggctggtgctttccatcgtcggtttggcggtggtgatggcaggcggtgtgaagcccagcaatctgggtttctcccacggcgccatcgggctcacggcgctgggcctgggactgctgcagccgctgaacgccttcttccgcccgcacaagggcgagcgctggcgcatgcagtgggagtggctgcacctcaccaccggcaggtgtgcggtggtgctgggggcggcaaacgtgagcctgggcaccttcctggtgcagggcccctacgcggtgtggatctcgtggcacgtcctgctgggcgtgttcgtcatcgtggtcatcattatggaggttcggcatcagcgggatctgcggcgcaatgctgggcgcccagatgccgcagccgcacccgccaaggatgttgacacatcaaccgactctgctgaagctggaaccagtggcggcacatctggtggcaaggcatctgtcgcacctgcaccgagcaagctggcctag

SEQ ID NO: 104 - protein, 639 aa, Chlamydomonas reinhardtii
MRVSLLLSVAFLSVLALPSATKLSWSRLQTSAANQPSPRRGAALVHDAFTEQLFLFGGSDGESVALNDVWVFQLNNQSWRQLNVTGPRPAGRHTFVYGLYVNSSSSASGSSTAARPTRVLVVATGQGSAVFSDIWALDLDSVTWRQLPQAGDPPNTVYGSAGGIAPSVPGGPHSSRFWLSHGFSSKRRYSTTHYYDLQAERWVLVHGAINSYDATGPHSRCIVSSTVMSDERIVMYGGCAQNGGTGGPCPARDAWTFDGSSWKQASTCPTPRTRGVMVPLTSPLLSLPTSADMAAAAAQEAMMGPGRTGSGGIMGPDSLNQTAGLRVYGGRYVLLYGGYERDKQTISVSSAPDDQLPVLDLDNGEWLLLRASGEVPAFRGQPAVAHDVATGRVWVFGGQLRGSGALSNDLYELQGDPEATPPYPDGSCGSTFLYPHLHGIFMGLAWGILLQAGWFIARYFKRSTTWFNLHRACQISGLVLSIVGLAVVMAGGVKPSNLGFSHGAIGLTALGLGLLQPLNAFFRPHKGERWRMQWEWLHLTTGRCAVVLGAANVSLGTFLVQGPYAVWISWHVLLGVFVIVVIIMEVRHQRDLRRNAGRPDAAAAPAKDVDTSTDSAEAGTSGGTSGGKASVAPAPSKLA

SEQ ID NO: 105 - DNA, 1941 bp, artificial sequence
  codon optimized
catatgctcgagcgtgtgtcgctcctgctgtcggtggcatttctctccgtgctggctctgccgagcgccaccaagctgagctggtcccgtctccagacctcggccgctaaccagccgagcccacggcggggtgccgccctcgtgcatgacgcattcaccgagcagctgttcctgtttggcggctccgacggcgagagcgtggcgctcaacgatgtgtgggtgttccagctgaataaccagtcctggcgtcagctgaacgtgacaggcccacgccccgctggtcgccacaccttcgtgtacggcctgtacgtgaacagctccagcagcgcgtccggcagcagcacggccgctcggcccactcgcgtgctggtggtcgctaccgggcagggctcggcggtgttcagcgacatttgggcactggatctggatagcgtgacttggcgccagctgccccaggcaggtgatcccccgaacacagtgtacggcagcgctggcggcattgcaccttccgtgcctggcggtccccactcgtcgcgcttctggctgagccacggcttctcgtcgaagcggcgctactcgaccacgcactactacgacctccaggcggagcgctgggtcctcgtccacggcgcgatcaactcctacgacgcaactggcccccacagccgctgcatcgtgtcgagcaccgtgatgtccgacgagcgtatcgtcatgtacgggggctgtgcgcagaacggcggcacaggcggtccatgccctgcgcgcgacgcctggacgttcgacggttcctcctggaagcaggcttcgacctgcccgacccctcgtacgcgtggcgtgatggtccccctgacttcccctctgctgagcctgcccacctcggcggacatggctgcggcagccgctcaggaggctatgatgggtcccggccgcaccggctccggcggcatcatgggccccgacagcctgaaccagactgcgggcctgcgggtgtacggcggtcgctacgtgctgctgtatggcggttacgagcgcgacaagcagaccatcagcgtgtccagcgctcccgacgaccaactgccggtgctggacctggacaacggtgagtggctgctgctgcgtgcaagcggcgaggtcccggccttccgcggccagcccgcggtcgcacacgacgtggcgaccggccgtgtctgggtcttcggcgggcaactgcgggggagcggtgcgctgagcaacgacctgtacgagctccagggcgatcccgaggccactccgccgtaccctgacggttcgtgcggctccacgtttctgtatccgcacctgcacggcatcttcatggggctggcgtggggcatcctgctgcaagcgggttggttcattgcgcgctacttcaagcgttccacgacatggtttaacctgcaccgcgcgtgccagattagcggcctggtgctgagcatcgtgggcctggccgtggtgatggctggcggcgtgaagccgtcgaacctggggttcagccacggcgcaattggcctgaccgccctgggcctcggcctgctccagcccctgaacgcctttttccgcccccacaagggcgagcggtggcggatgcagtgggagtggctccacctgaccacgggccgctgcgctgtggtcctgggcgctgcgaacgtgtccctgggtacgttcctggtgcagggcccttacgcggtctggattagctggcacgtcctgctgggggtgttcgtcatcgtcgtgatcatcatggaggtgcgccaccagcgggacctgcgccgcaacgccggtcgcccggacgccgccgctgcaccggcgaaggacgtggacacgagcacggacagcgccgaggcggggacctccggtggcacttcgggcggcaaggcctccgtcgcaccggccccttccaagctggccaccggttaaggatcc

SEQ ID NO: 106 - DNA, 1914 bp, artificial sequence
  codon optimized
cgtgtgtcgctcctgctgtcggtggcatttctctccgtgctggctctgccgagcgccaccaagctgagctggtcccgtctccagacctcggccgctaaccagccgagcccacggcggggtgccgccctcgtgcatgacgcattcaccgagcagctgttcctgtttggcggctccgacggcgagagcgtggcgctcaacgatgtgtgggtgttccagctgaataaccagtcctggcgtcagctgaacgtgacaggcccacgccccgctggtcgccacaccttcgtgtacggcctgtacgtgaacagctccagcagcgcgtccggcagcagcacggccgctcggcccactcgcgtgctggtggtcgctaccgggcagggctcggcggtgttcagcgacatttgggcactggatctggatagcgtgacttggcgccagctgccccaggcaggtgatcccccgaacacagtgtacggcagcgctggcggcattgcaccttccgtgcctggcggtccccactcgtcgcgcttctggctgagccacggcttctcgtcgaagcggcgctactcgaccacgcactactacgacctccaggcggagcgctgggtcctcgtccacggcgcgatcaactcctacgacgcaactggcccccacagccgctgcatcgtgtcgagcaccgtgatgtccgacgagcgtatcgtcatgtacgggggctgtgcgcagaacggcggcacaggcggtccatgccctgcgcgcgacgcctggacgttcgacggttcctcctggaagcaggcttcgacctgcccgacccctcgtacgcgtggcgtgatggtccccctgacttcccctctgctgagcctgcccacctcggcggacatggctgcggcagccgctcaggaggctatgatgggtcccggccgcaccggctccggcggcatcatgggccccgacagcctgaaccagactgcgggcctgcgggtgtacggcggtcgctacgtgctgctgtatggcggttacgagcgcgacaagcagaccatcagcgtgtccagcgctcccgacgaccaactgccggtgctggacctggacaacggtgagtggctgctgctgcgtgcaagcggcgaggtcccggccttccgcggccagcccgcggtcgcacacgacgtggcgaccggccgtgtctgggtcttcggcgggcaactgcgggggagcggtgcgctgagcaacgacctgtacgagctccagggcgatcccgaggccactccgccgtaccctgacggttcgtgcggctccacgtttctgtatccgcacctgcacggcatcttcatggggctggcgtggggcatcctgctgcaagcgggttggttcattgcgcgctacttcaagcgttccacgacatggtttaacctgcaccgcgcgtgccagattagcggcctggtgctgagcatcgtgggcctggccgtggtgatggctggcggcgtgaagccgtcgaacctggggttcagccacggcgcaattggcctgaccgccctgggcctcggcctgctccagcccctgaacgcctttttccgcccccacaagggcgagcggtggcggatgcagtgggagtggctccacctgaccacgggccgctgcgctgtggtcctgggcgctgcgaacgtgtccctgggtacgttcctggtgcagggcccttacgcggtctggattagctggcacgtcctgctgggggtgttcgtcatcgtcgtgatcatcatggaggtgcgccaccagcgggacctgcgccgcaacgccggtcgcccggacgccgccgctgcaccggcgaaggacgtggacacgagcacggacagcgccgaggcggggacctccggtggcacttcgggcggcaaggcctccgtcgcaccggccccttccaagctggcc

SEQ ID NO: 107 - DNA, 1914 bp, Chlamydomonas reinhardtii
cgggtcagtcttcttctgtctgtggcgttcttgtctgtgctggcgctcccaagcgccacaaagctatcgtggtcacgactgcagacgtcagctgcgaaccagccctcgcctcggcgtggagctgcccttgtgcacgatgcgttcacagagcagttgttccttttcggtggcagcgatggcgagtcagtcgcgctgaacgatgtttgggtcttccagctgaacaaccagagttggcgacaactcaacgtgaccgggcctcgcccagctggccggcacaccttcgtgtacggcctgtacgtcaacagcagcagtagtgcctcgggcagcagcacggctgcgcgccccacacgtgtgctggttgtggccactgggcagggctccgccgtgttctccgacatatgggccctggatctggattcggtcacgtggcgacagctgccgcaagcgggggatccgcccaacacggtgtacggctccgcaggcggcatcgcgccctcggtcccgggcggcccgcacagcagccgcttctggctgtcgcacggcttctcctccaagcggcgctactcaaccactcactactatgacctacaggcggagaggtgggtccttgtgcacggcgccataaactcgtacgatgctacgggcccgcactcacgctgcatcgtgagcagcaccgtcatgagcgacgagcggatcgtcatgtacggaggctgcgcccagaacggaggcacgggcggcccctgccctgcgcgcgacgcctggacgtttgacgggtcaagctggaagcaggcctccacctgtcccacaccccgcacacgtggcgtcatggtgcccctcacgtcccctctcctgtcgctgcccacatcggctgacatggccgccgcggcggctcaggaggcgatgatggggccgggccgaacgggcagtggtggcataatggggcccgacagcctgaaccaaaccgccgggctgcgagtgtacggcgggcgttacgtgcttctgtacggcggttacgagcgcgacaagcagaccatctccgtgtcgtcggcaccagacgaccagctcccggtgctggatcttgacaatggcgagtggctgcttctgcgcgcgtcgggggaggtcccggcattcaggggccagcccgcggtcgcacacgacgtggcgaccgggcgtgtgtgggtgtttggcggccagctccgcggcagtggcgccctcagcaacgacctgtacgagctgcagggcgacccggaggccacgccgccctacccggatggctcatgtggctccaccttcctatacccgcatctgcacggcatatttatgggccttgcgtggggcatactgctacaggcaggctggtttatcgcgcgctacttcaagcggagcacgacttggttcaatctgcatcgtgcctgtcagatttccgggctggtgctttccatcgtcggtttggcggtggtgatggcaggcggtgtgaagcccagcaatctgggtttctcccacggcgccatcgggctcacggcgctgggcctgggactgctgcagccgctgaacgccttcttccgcccgcacaagggcgagcgctggcgcatgcagtgggagtggctgcacctcaccaccggcaggtgtgcggtggtgctgggggcggcaaacgtgagcctgggcaccttcctggtgcagggcccctacgcggtgtggatctcgtggcacgtcctgctgggcgtgttcgtcatcgtggtcatcattatggaggttcggcatcagcgggatctgcggcgcaatgctgggcgcccagatgccgcagccgcacccgccaaggatgttgacacatcaaccgactctgctgaagctggaaccagtggcggcacatctggtggcaaggcatctgtcgcacctgcaccgagcaagctggcc

SEQ ID NO: 108 - protein, 638 aa, Chlamydomonas reinhardtii
RVSLLLSVAFLSVLALPSATKLSWSRLQTSAANQPSPRRGAALVHDAFTEQLFLFGGSDGESVALNDVWVFQLNNQSWRQLNVTGPRPAGRHTFVYGLYVNSSSSASGSSTAARPTRVLVVATGQGSAVFSDIWALDLDSVTWRQLPQAGDPPNTVYGSAGGIAPSVPGGPHSSRFWLSHGFSSKRRYSTTHYYDLQAERWVLVHGAINSYDATGPHSRCIVSSTVMSDERIVMYGGCAQNGGTGGPCPARDAWTFDGSSWKQASTCPTPRTRGVMVPLTSPLLSLPTSADMAAAAAQEAMMGPGRTGSGGIMGPDSLNQTAGLRVYGGRYVLLYGGYERDKQTISVSSAPDDQLPVLDLDNGEWLLLRASGEVPAFRGQPAVAHDVATGRVWVFGGQLRGSGALSNDLYELQGDPEATPPYPDGSCGSTFLYPHLHGIFMGLAWGILLQAGWFIARYFKRSTTWFNLHRACQISGLVLSIVGLAVVMAGGVKPSNLGFSHGAIGLTALGLGLLQPLNAFFRPHKGERWRMQWEWLHLTTGRCAVVLGAANVSLGTFLVQGPYAVWISWHVLLGVFVIVVIIMEVRHQRDLRRNAGRPDAAAAPAKDVDTSTDSAEAGTSGGTSGGKASVAPAPSKLA

SEQ ID NO: 109 - DNA, 2223 bp, Chlamydomonas reinhardtii
atgcgttctctgatagcgtgcgaatgcgtgtgcggatccgggagcacgcacggccatgccagtagtaacaccaaggcggaccgtattgacacacatgctctgctacacgtcggcacatgtggggagccctgcaaccactcatacgacgcggtagacgcaacggccaccgccaccgcaacaaccttccgcaactttggtgcttcccgcggcagcagcgggtcaccacaatcccgcggtacagctccattccaaacgagcgccggcggcttctgcaatacgcacggcgttcacagcaacggcggaagcgggggcgcggccggcagcccagaggtcggcgctggccacaagcgtggctcttcgccttctcgtgtgcacaagtaccaccaaccgccgccggctggtgagcctttggactactgccagccgccggcatttgcaatgcctgcaaccctgccgcggcagcggacgcagcgctcatgccagccgacgacgccgcaccaggcgtcggagtcgcaggaccagcagcagcatccacagcagcggtggaagggcgggagcacggctgctgcagcggatgaggactgtgctgcagatgacctgctgctggtcaatcggggggctgtgggcctactggatttgagcggccgcgcgggggagctggtgcgcctggggcaaggcctcgcgcacaagctcaagggcgtacagtctgcgcgcgcctcgctgcaacggctggagttggagctgggccatgcgctaggtcgcctgaggtcccagctgcaggcaacactgcggcagcagcacccacaccagctgctaccgcccgagtggatacagctgctacagcccccgcccccgctgctggcggttgcagcggcagggaacgcggaaatggaggacagctcaccaccgccacgcagtgcgctgccgtcaccacctcaaatgctacagccgcagccgagttcaacgccgccgcgagcgccggaggaactgccgctgccattggcggctcctcagtcgccattttgggagcagcgggcggcggtggccgccacgaccgccactgccagcggcggtcatcccgggtgtaatgctggtacggctgcggccggtgctgccgcagccgctggcgcctctaccgccgctcgcacctctgcctccgcccccgcttgtgccgccagcacggctagtctacagcggcaggagcgcacggcgggtggcggcgtagctcaaggaggtgcccagccggcaattgctccgcccacgtcgcagccgcctgcatcgccacctgcgtcgcagccccgctgctggcccctcggcgtgttgccacccatggagctcgtctcgtccggcggagcgccagcggcgcgcgtcacgccattggggcgacgagcgtatgcagctgccgtagccgccgccagtggtgtccctgcaccggtggatggcagcagcagtaggagcaggaacaagcgtgcattggattcaaccagtgctgagaaaggaggaggagaggaggcagccctgaggttgctggctgcggcagccgcagccgcagccgcagccgccgtcacggcaggcgaccagccgccgctgaatgcgcgccgccgcagccatgcagctagggaaaccacgcagccacctaagccatcaccctcaaagctgccgacagcggcacagcaggcggcgccacagcctcgtgacggcggcagctttggtaacactagcagcggcggcgccgccgccgtccctgccaccgccagcactggcgcagccgtgcccatgtcgacgatcgacgccgccgccgctgccgccatcgacccaatgctttgggacgagtcgcccgcagatgacgtgctgcgggctgcggctgcagttatgaagtctgcatctgtggctgccgccgccgccgctgcacctgcgtcggcgtcagcgtccgtgcagctcccggcgcattcgtcagcaaagcgcccgcgctatagcgacagcggtgctgaaagcggtggcagggcgcaaggtggcgagatggtggcggcagcggcagcggcagcggcgctgtcttcggcaatgatgccaccgccgcggccaaggcctgcagcggctgcggcgagggagcagccggcaggcatgccggtcgccgcggcggcggcggcgagagtgattgaggagcatatcgatgtgattggcctgctccggggcctgctccatcaacatcagggaggaaagtag

SEQ ID NO: 110 - protein, 740 aa, Chlamydomonas reinhardtii
MRSLIACECVCGSGSTHGHASSNTKADRIDTHALLHVGTCGEPCNHSYDAVDATATATATTFRNFGASRGSSGSPQSRGTAPFQTSAGGFCNTHGVHSNGGSGGAAGSPEVGAGHKRGSSPSRVHKYHQPPPAGEPLDYCQPPAFAMPATLPRQRTQRSCQPTTPHQASESQDQQQHPQQRWKGGSTAAAADEDCAADDLLLVNRGAVGLLDLSGRAGELVRLGQGLAHKLKGVQSARASLQRLELELGHALGRLRSQLQATLRQQHPHQLLPPEWIQLLQPPPPLLAVAAAGNAEMEDSSPPPRSALPSPPQMLQPQPSSTPPRAPEELPLPLAAPQSPFWEQRAAVAATTATASGGHPGCNAGTAAAGAAAAAGASTAARTSASAPACAASTASLQRQERTAGGGVAQGGAQPAIAPPTSQPPASPPASQPRCWPLGVLPPMELVSSGGAPAARVTPLGRRAYAAAVAAASGVPAPVDGSSSRSRNKRALDSTSAEKGGGEEAALRLLAAAAAAAAAAAVTAGDQPPLNARRRSHAARETTQPPKPSPSKLPTAAQQAAPQPRDGGSFGNTSSGGAAAVPATASTGAAVPMSTIDAAAAAAIDPMLWDESPADDVLRAAAAVMKSASVAAAAAAAPASASASVQLPAHSSAKRPRYSDSGAESGGRAQGGEMVAAAAAAAALSSAMMPPPRPRPAAAAAREQPAGMPVAAAAAARVIEEHIDVIGLLRGLLHQHQGGK

SEQ ID NO: 111 - DNA, 2238 bp, artificial sequence
  codon optimized
catatgctcgagcgtagcctcatcgcctgcgagtgcgtgtgcggttccggctcgacccacggccacgcgtcgagcaacactaaggccgatcgcatcgacacgcacgccctgctccacgtgggcacctgcggcgagccgtgcaaccacagctacgatgcagtggatgccactgccaccgcaaccgcgaccaccttccgcaacttcggcgcgtcgcggggcagcagcggctcgccccagagccggggcacggcgccttttcagacgagcgcaggcggtttctgcaacacgcacggcgtgcacagcaacggcgggtcgggcggcgctgcgggttcccccgaggtcggtgcgggccacaagcgcggcagctcgccctcgcgcgtgcacaagtaccaccagccacccccagccggcgagcccctggactactgccagcctcccgccttcgccatgcccgcgacgctgccccgccagcgcacacaacgctcgtgccaaccgaccacgccccaccaggcctcggagtcgcaggaccagcagcagcatccccagcagcgctggaagggcggctcgacagccgcagccgcagacgaggactgtgcggccgacgacctgctcctggtgaaccggggtgctgtgggtctgctggacctgtccggtcgcgcgggtgagctggtgcgcctgggtcaaggtctggcgcacaagctgaagggcgtgcagtccgcccgtgccagcctccagcggctggagctggagctggggcacgccctgggccgcctccgctcccagctccaggccaccctgcgccagcagcacccgcaccagctgctgccccctgagtggattcagctgctccagcccccacccccgctgctggcggtggcagcggccggtaacgctgagatggaggacagcagcccacctccgcgctccgcgctgccgagccctccacagatgctccagccgcagccgtcctccacacctccacgtgcgccggaggagctcccgctgcccctggcggctccgcagtcccccttttgggagcaacgcgccgcagtcgcggccaccaccgccacggcgagcggtggccaccccggctgcaacgccggcaccgccgctgcgggcgctgcggctgccgccggtgcctcgacggcggctcgcactagcgcctccgcgccagcgtgcgccgcgagcacggcttccctccagcgccaggagcgcaccgccggtggcggtgtggcccagggtggtgcgcagcccgctattgcgcctccgacctcgcaaccacctgcctcccctcccgcctcccaaccccgctgctggccgctgggcgtgctgcccccaatggagctggtgtcgtccggtggcgcgcctgctgctcgcgtcacgcccctcggtcgccgcgcttatgcggcggctgtggcagccgcttcgggcgtgcctgccccggtggacggctccagcagccggagccgcaacaagcgggcgctggactcgacctcggcggagaagggcggtggcgaggaggctgcgctgcggctgctcgcagccgctgccgcggcagctgccgccgcagcggtgactgccggcgaccagccgcccctgaacgctcgccggcgcagccacgctgcgcgggagacgacgcagcccccgaagccgtcgccctcgaagctgcccacagcggctcagcaggctgcaccgcagccccgcgacggcggctccttcggcaacaccagcagcggtggtgctgccgcggtgcctgccaccgcgtccactggtgcggcggtgcccatgagcacgattgacgccgctgccgctgccgcgatcgaccctatgctgtgggacgagagccctgcggacgacgtgctgcgcgctgccgccgctgtgatgaagagcgccagcgtcgcagcggcggcagccgcggctcccgcatcggcgtccgcgtccgtgcagctgcccgcccacagctccgcgaagcggcctcgctactccgattccggtgccgagtccggcggtcgcgcgcagggcggtgagatggtcgccgctgccgcagccgcagccgccctgagcagcgctatgatgcctccccctcgccctcgccccgctgctgctgccgcgcgtgagcaacccgcaggtatgccggtcgccgctgctgccgccgcacgcgtgattgaggagcacatcgacgtgattggcctgctgcgcggcctgctgcaccagcaccagggcggcaagtagggatcc

SEQ ID NO: 112 - DNA, 2217 bp, artificial sequence
  codon optimized
cgtagcctcatcgcctgcgagtgcgtgtgcggttccggctcgacccacggccacgcgtcgagcaacactaaggccgatcgcatcgacacgcacgccctgctccacgtgggcacctgcggcgagccgtgcaaccacagctacgatgcagtggatgccactgccaccgcaaccgcgaccaccttccgcaacttcggcgcgtcgcggggcagcagcggctcgccccagagccggggcacggcgccttttcagacgagcgcaggcggtttctgcaacacgcacggcgtgcacagcaacggcgggtcgggcggcgctgcgggttcccccgaggtcggtgcgggccacaagcgcggcagctcgccctcgcgcgtgcacaagtaccaccagccacccccagccggcgagcccctggactactgccagcctcccgccttcgccatgcccgcgacgctgccccgccagcgcacacaacgctcgtgccaaccgaccacgccccaccaggcctcggagtcgcaggaccagcagcagcatccccagcagcgctggaagggcggctcgacagccgcagccgcagacgaggactgtgcggccgacgacctgctcctggtgaaccggggtgctgtgggtctgctggacctgtccggtcgcgcgggtgagctggtgcgcctgggtcaaggtctggcgcacaagctgaagggcgtgcagtccgcccgtgccagcctccagcggctggagctggagctggggcacgccctgggccgcctccgctcccagctccaggccaccctgcgccagcagcacccgcaccagctgctgccccctgagtggattcagctgctccagcccccacccccgctgctggcggtggcagcggccggtaacgctgagatggaggacagcagcccacctccgcgctccgcgctgccgagccctccacagatgctccagccgcagccgtcctccacacctccacgtgcgccggaggagctcccgctgcccctggcggctccgcagtcccccttttgggagcaacgcgccgcagtcgcggccaccaccgccacggcgagcggtggccaccccggctgcaacgccggcaccgccgctgcgggcgctgcggctgccgccggtgcctcgacggcggctcgcactagcgcctccgcgccagcgtgcgccgcgagcacggcttccctccagcgccaggagcgcaccgccggtggcggtgtggcccagggtggtgcgcagcccgctattgcgcctccgacctcgcaaccacctgcctcccctcccgcctcccaaccccgctgctggccgctgggcgtgctgcccccaatggagctggtgtcgtccggtggcgcgcctgctgctcgcgtcacgcccctcggtcgccgcgcttatgcggcggctgtggcagccgcttcgggcgtgcctgccccggtggacggctccagcagccggagccgcaacaagcgggcgctggactcgacctcggcggagaagggcggtggcgaggaggctgcgctgcggctgctcgcagccgctgccgcggcagctgccgccgcagcggtgactgccggcgaccagccgcccctgaacgctcgccggcgcagccacgctgcgcgggagacgacgcagcccccgaagccgtcgccctcgaagctgcccacagcggctcagcaggctgcaccgcagccccgcgacggcggctccttcggcaacaccagcagcggtggtgctgccgcggtgcctgccaccgcgtccactggtgcggcggtgcccatgagcacgattgacgccgctgccgctgccgcgatcgaccctatgctgtgggacgagagccctgcggacgacgtgctgcgcgctgccgccgctgtgatgaagagcgccagcgtcgcagcggcggcagccgcggctcccgcatcggcgtccgcgtccgtgcagctgcccgcccacagctccgcgaagcggcctcgctactccgattccggtgccgagtccggcggtcgcgcgcagggcggtgagatggtcgccgctgccgcagccgcagccgccctgagcagcgctatgatgcctccccctcgccctcgccccgctgctgctgccgcgcgtgagcaacccgcaggtatgccggtcgccgctgctgccgccgcacgcgtgattgaggagcacatcgacgtgattggcctgctgcgcggcctgctgcaccagcaccagggcggcaag

SEQ ID NO: 113 - DNA, 2217 bp, Chlamydomonas reinhardtii
cgttctctgatagcgtgcgaatgcgtgtgcggatccgggagcacgcacggccatgccagtagtaacaccaaggcggaccgtattgacacacatgctctgctacacgtcggcacatgtggggagccctgcaaccactcatacgacgcggtagacgcaacggccaccgccaccgcaacaaccttccgcaactttggtgcttcccgcggcagcagcgggtcaccacaatcccgcggtacagctccattccaaacgagcgccggcggcttctgcaatacgcacggcgttcacagcaacggcggaagcgggggcgcggccggcagcccagaggtcggcgctggccacaagcgtggctcttcgccttctcgtgtgcacaagtaccaccaaccgccgccggctggtgagcctttggactactgccagccgccggcatttgcaatgcctgcaaccctgccgcggcagcggacgcagcgctcatgccagccgacgacgccgcaccaggcgtcggagtcgcaggaccagcagcagcatccacagcagcggtggaagggcgggagcacggctgctgcagcggatgaggactgtgctgcagatgacctgctgctggtcaatcggggggctgtgggcctactggatttgagcggccgcgcgggggagctggtgcgcctggggcaaggcctcgcgcacaagctcaagggcgtacagtctgcgcgcgcctcgctgcaacggctggagttggagctgggccatgcgctaggtcgcctgaggtcccagctgcaggcaacactgcggcagcagcacccacaccagctgctaccgcccgagtggatacagctgctacagcccccgcccccgctgctggcggttgcagcggcagggaacgcggaaatggaggacagctcaccaccgccacgcagtgcgctgccgtcaccacctcaaatgctacagccgcagccgagttcaacgccgccgcgagcgccggaggaactgccgctgccattggcggctcctcagtcgccattttgggagcagcgggcggcggtggccgccacgaccgccactgccagcggcggtcatcccgggtgtaatgctggtacggctgcggccggtgctgccgcagccgctggcgcctctaccgccgctcgcacctctgcctccgcccccgcttgtgccgccagcacggctagtctacagcggcaggagcgcacggcgggtggcggcgtagctcaaggaggtgcccagccggcaattgctccgcccacgtcgcagccgcctgcatcgccacctgcgtcgcagccccgctgctggcccctcggcgtgttgccacccatggagctcgtctcgtccggcggagcgccagcggcgcgcgtcacgccattggggcgacgagcgtatgcagctgccgtagccgccgccagtggtgtccctgcaccggtggatggcagcagcagtaggagcaggaacaagcgtgcattggattcaaccagtgctgagaaaggaggaggagaggaggcagccctgaggttgctggctgcggcagccgcagccgcagccgcagccgccgtcacggcaggcgaccagccgccgctgaatgcgcgccgccgcagccatgcagctagggaaaccacgcagccacctaagccatcaccctcaaagctgccgacagcggcacagcaggcggcgccacagcctcgtgacggcggcagctttggtaacactagcagcggcggcgccgccgccgtccctgccaccgccagcactggcgcagccgtgcccatgtcgacgatcgacgccgccgccgctgccgccatcgacccaatgctttgggacgagtcgcccgcagatgacgtgctgcgggctgcggctgcagttatgaagtctgcatctgtggctgccgccgccgccgctgcacctgcgtcggcgtcagcgtccgtgcagctcccggcgcattcgtcagcaaagcgcccgcgctatagcgacagcggtgctgaaagcggtggcagggcgcaaggtggcgagatggtggcggcagcggcagcggcagcggcgctgtcttcggcaatgatgccaccgccgcggccaaggcctgcagcggctgcggcgagggagcagccggcaggcatgccggtcgccgcggcggcggcggcgagagtgattgaggagcatatcgatgtgattggcctgctccggggcctgctccatcaacatcagggaggaaag

SEQ ID NO: 114 - protein, 739 aa, Chlamydomonas reinhardtii
RSLIACECVCGSGSTHGHASSNTKADRIDTHALLHVGTCGEPCNHSYDAVDATATATATTFRNFGASRGSSGSPQSRGTAPFQTSAGGFCNTHGVHSNGGSGGAAGSPEVGAGHKRGSSPSRVHKYHQPPPAGEPLDYCQPPAFAMPATLPRQRTQRSCQPTTPHQASESQDQQQHPQQRWKGGSTAAAADEDCAADDLLLVNRGAVGLLDLSGRAGELVRLGQGLAHKLKGVQSARASLQRLELELGHALGRLRSQLQATLRQQHPHQLLPPEWIQLLQPPPPLLAVAAAGNAEMEDSSPPPRSALPSPPQMLQPQPSSTPPRAPEELPLPLAAPQSPFWEQRAAVAATTATASGGHPGCNAGTAAAGAAAAAGASTAARTSASAPACAASTASLQRQERTAGGGVAQGGAQPAIAPPTSQPPASPPASQPRCWPLGVLPPMELVSSGGAPAARVTPLGRRAYAAAVAAASGVPAPVDGSSSRSRNKRALDSTSAEKGGGEEAALRLLAAAAAAAAAAAVTAGDQPPLNARRRSHAARETTQPPKPSPSKLPTAAQQAAPQPRDGGSFGNTSSGGAAAVPATASTGAAVPMSTIDAAAAAAIDPMLWDESPADDVLRAAAAVMKSASVAAAAAAAPASASASVQLPAHSSAKRPRYSDSGAESGGRAQGGEMVAAAAAAAALSSAMMPPPRPRPAAAAAREQPAGMPVAAAAAARVIEEHIDVIGLLRGLLHQHQGGK

SEQ ID NO: 115 - DNA, 1923 bp, Chlamydomonas reinhardtii
atgaacatcccggagcccctgctcgccgagcacccctcctggccgggcaccgctttcgtggctcactggggccgccccgagccctggcgtgtccttaaccggcgacaacggcatcgcctggtgtgcttggctgcctccagcctgcacccgcccagtctcgatgcggtgctagcacactgcggcgtccttccccgcgcagacgcgctggcctccgccgcggccgtgggcgacctgccgacatgccagcggctgctgctggaggagggctgccactgggacgcggcgaggctgtggcacgccgccgccggctacgggcacctgcatgtgtgcaaatggctggatgaaatgggcatggacaccgaatgggccatgtgcggcggtgatgtcttgagcttgggccgcggcggcgtgcccctcgcggtgcaggcggcggccgccaatggccaccgcgacgtactgcagtggctgcgggggatctggaagatacccgcggaagtcatcgcgggagcggccgcggaggggagccagatggggctgttctgggaacaggagcccgaggcggccggcgaccgtgtcggtcgccagctgctccttaaggtgctgcacggctgcccgctcagcgacctgcagcgcttctgcaaggcgcggaacctggaaagcgcgttgtctgacgcccccgacagcaccaagcacgatctgctgatgcgtgcggcgaccagccccacggccgactgggcggacaagtgcggctggctgctgtcgctgtggcgcaccccgccctcggaatgggcggagctagacgacgaaggcatcgaaggaaataagcttgtctacctttgggaacggctctggcaggccgtcgcgcggctgcagccggacacgttgctgccacgggtgcagtacctggtggctcgcgggctgcgcctgtcggagacggacgcgcttgtaccgcaccaggcagcggccgccgccggccacatggcaggggtggccatggcactgcgcgccgggccgccgctgctgcagcttcagggtccgtggggcagcggcaccgctgaggacccggcgcggcggggactgcggcgccacgtgtctagctttctctcctccgtcgcaattaaggcgggccaggcgtccgtccttccgttgtggggccgcgtcttcatgcctccgatcttaaaatgggccctgaagaatgtctgggacacgtatggcgacgtcgtggcttcccaaacggaggaccctttccaaggcctgcccgctgtgcgctaccttgtgatggaggaccaggtgtgcaagccagccaagggcgagcccggccccgactggtcggcgatcttccggcatgtggcgcgaagcggcgcggacttaccgctgctgcgcttcctacatgagcggcgtggcgcggccatcgacctggcggcggtagcgcgcggcggcggcgaggagcagctggagtgggcgctgggcgccctgcacgcagccggccagccatgtgacgagcctctggccgaggaccagtttatggcgattctggaggccggcaactgggcggcggcggactggctgctgcaccgcaggctggcaccagaacccctggagctgcagtttgacctgctgcagcacatcggagcgctcaaccggttggtcccggcgctgcgctggtttgtgagccgccacgaagggctgcgctggctgtcggacatggcagccgccgttaaggagaagtacagcgggggggcagcggagcaggaagacagcccggaggaagaatcggaggaagaatcggaggaagagtcggaggaagaatcggaggatagcgaggaggcgggtgaggaggcgggtgtgcaggaggacgcacaagagggcgcgcacgcggctgcaagcgcgggtgtgggcggcggtgctgctggagacacatga

SEQ ID NO: 116 - protein, 640 aa, Chlamydomonas reinhardtii
MNIPEPLLAEHPSWPGTAFVAHWGRPEPWRVLNRRQRHRLVCLAASSLHPPSLDAVLAHCGVLPRADALASAAAVGDLPTCQRLLLEEGCHWDAARLWHAAAGYGHLHVCKWLDEMGMDTEWAMCGGDVLSLGRGGVPLAVQAAAANGHRDVLQWLRGIWKIPAEVIAGAAAEGSQMGLFWEQEPEAAGDRVGRQLLLKVLHGCPLSDLQRFCKARNLESALSDAPDSTKHDLLMRAATSPTADWADKCGWLLSLWRTPPSEWAELDDEGIEGNKLVYLWERLWQAVARLQPDTLLPRVQYLVARGLRLSETDALVPHQAAAAAGHMAGVAMALRAGPPLLQLQGPWGSGTAEDPARRGLRRHVSSFLSSVAIKAGQASVLPLWGRVFMPPILKWALKNVWDTYGDVVASQTEDPFQGLPAVRYLVMEDQVCKPAKGEPGPDWSAIFRHVARSGADLPLLRFLHERRGAAIDLAAVARGGGEEQLEWALGALHAAGQPCDEPLAEDQFMAILEAGNWAAADWLLHRRLAPEPLELQFDLLQHIGALNRLVPALRWFVSRHEGLRWLSDMAAAVKEKYSGGAAEQEDSPEEESEEESEEESEEESEDSEEAGEEAGVQEDAQEGAHAAASAGVGGGAAGDT

SEQ ID NO: 117 - DNA, 1938 bp, artificial sequence
  codon optimized
catatgctcgagaacatccccgagccactcctggcagagcacccgagctggcccggcaccgcgttcgtcgcgcactggggccgtcctgagccgtggcgtgtgctcaaccgccggcagcgccaccggctggtgtgcctggccgccagcagcctgcatccgccctcgctggacgctgtcctggcccactgcggcgtgctcccccgtgctgatgccctggcctccgctgcggcggtcggcgacctgccgacctgccagcgcctcctgctggaggaggggtgtcactgggacgcagctcggctgtggcacgccgcagccgggtacggccatctgcacgtgtgcaagtggctggacgagatgggcatggacaccgagtgggcgatgtgcggtggcgacgtgctgtcgctgggtcgcggtggcgtccccctggcggtccaagccgccgcagctaacggccaccgcgacgtgctccagtggctgcgcggtatttggaagatcccggccgaggtgatcgccggtgcggcggcagagggctcgcagatgggcctgttctgggagcaggagccggaggccgctggggaccgcgtgggccgccagctgctgctgaaggtgctgcacggctgccccctgagcgatctccagcgcttctgcaaggcgcgcaatctggagagcgctctgtcggacgcgcccgactccaccaagcacgacctgctcatgcgcgctgccacgagccccaccgcggactgggcggataagtgtggctggctgctgagcctctggcgcaccccgccctccgagtgggcggagctcgacgacgaggggatcgagggcaacaagctggtgtatctgtgggagcgcctctggcaagctgtggcccggctgcaacctgacaccctgctgcctcgcgtgcagtacctcgtggcccgtgggctccgcctgtcggagacagacgcgctggtgccgcaccaggcagcggctgccgccggtcacatggcgggggtggcgatggctctccgcgctgggcctccgctgctgcaactccagggcccgtggggctccggcaccgcggaggacccggcacgccgtggcctgcgccgccacgtctccagcttcctgtccagcgtggcgatcaaggcgggccaggcgagcgtgctgcccctgtggggtcgcgtgtttatgccacccatcctcaagtgggcgctgaagaatgtgtgggacacctacggcgacgtggtggcttcgcagaccgaggaccccttccagggcctgcctgctgtgcgctacctggtgatggaggaccaggtgtgcaagcctgcgaagggcgagcctggcccggactggtcggctatttttcgccacgtcgcccgcagcggtgccgacctgcccctgctgcgcttcctgcacgagcgtcgtggcgctgccatcgacctcgcagccgtggcacgtggcggtggtgaggagcagctggagtgggccctgggggcactccacgctgccgggcaaccgtgcgacgagccgctggccgaggatcagttcatggccatcctggaggctgggaactgggccgcagcagactggctgctgcaccggcgcctggcccccgagcccctggagctgcaattcgacctgctccagcacatcggtgcgctgaaccgtctcgtgcctgcgctgcgctggttcgtctcgcgccacgagggcctgcgctggctctccgacatggcggctgctgtgaaggagaagtacagcggcggtgccgccgagcaggaggactcgcccgaggaggagtccgaggaggagtcggaggaggagagcgaggaggagagcgaggactccgaggaggccggcgaggaggcgggtgtccaggaggatgcgcaggagggcgctcacgcggcagccagcgctggcgtgggtggcggtgcggccggtgacacctaaggatcc

SEQ ID NO: 118 - DNA, 1917 bp, artificial sequence
  codon optimized
aacatccccgagccactcctggcagagcacccgagctggcccggcaccgcgttcgtcgcgcactggggccgtcctgagccgtggcgtgtgctcaaccgccggcagcgccaccggctggtgtgcctggccgccagcagcctgcatccgccctcgctggacgctgtcctggcccactgcggcgtgctcccccgtgctgatgccctggcctccgctgcggcggtcggcgacctgccgacctgccagcgcctcctgctggaggaggggtgtcactgggacgcagctcggctgtggcacgccgcagccgggtacggccatctgcacgtgtgcaagtggctggacgagatgggcatggacaccgagtgggcgatgtgcggtggcgacgtgctgtcgctgggtcgcggtggcgtccccctggcggtccaagccgccgcagctaacggccaccgcgacgtgctccagtggctgcgcggtatttggaagatcccggccgaggtgatcgccggtgcggcggcagagggctcgcagatgggcctgttctgggagcaggagccggaggccgctggggaccgcgtgggccgccagctgctgctgaaggtgctgcacggctgccccctgagcgatctccagcgcttctgcaaggcgcgcaatctggagagcgctctgtcggacgcgcccgactccaccaagcacgacctgctcatgcgcgctgccacgagccccaccgcggactgggcggataagtgtggctggctgctgagcctctggcgcaccccgccctccgagtgggcggagctcgacgacgaggggatcgagggcaacaagctggtgtatctgtgggagcgcctctggcaagctgtggcccggctgcaacctgacaccctgctgcctcgcgtgcagtacctcgtggcccgtgggctccgcctgtcggagacagacgcgctggtgccgcaccaggcagcggctgccgccggtcacatggcgggggtggcgatggctctccgcgctgggcctccgctgctgcaactccagggcccgtggggctccggcaccgcggaggacccggcacgccgtggcctgcgccgccacgtctccagcttcctgtccagcgtggcgatcaaggcgggccaggcgagcgtgctgcccctgtggggtcgcgtgtttatgccacccatcctcaagtgggcgctgaagaatgtgtgggacacctacggcgacgtggtggcttcgcagaccgaggaccccttccagggcctgcctgctgtgcgctacctggtgatggaggaccaggtgtgcaagcctgcgaagggcgagcctggcccggactggtcggctatttttcgccacgtcgcccgcagcggtgccgacctgcccctgctgcgcttcctgcacgagcgtcgtggcgctgccatcgacctcgcagccgtggcacgtggcggtggtgaggagcagctggagtgggccctgggggcactccacgctgccgggcaaccgtgcgacgagccgctggccgaggatcagttcatggccatcctggaggctgggaactgggccgcagcagactggctgctgcaccggcgcctggcccccgagcccctggagctgcaattcgacctgctccagcacatcggtgcgctgaaccgtctcgtgcctgcgctgcgctggttcgtctcgcgccacgagggcctgcgctggctctccgacatggcggctgctgtgaaggagaagtacagcggcggtgccgccgagcaggaggactcgcccgaggaggagtccgaggaggagtcggaggaggagagcgaggaggagagcgaggactccgaggaggccggcgaggaggcgggtgtccaggaggatgcgcaggagggcgctcacgcggcagccagcgctggcgtgggtggcggtgcggccggtgacacc

SEQ ID NO: 119 - DNA, 1917 bp, Chlamydomonas reinhardtii
aacatcccggagcccctgctcgccgagcacccctcctggccgggcaccgctttcgtggctcactggggccgccccgagccctggcgtgtccttaaccggcgacaacggcatcgcctggtgtgcttggctgcctccagcctgcacccgcccagtctcgatgcggtgctagcacactgcggcgtccttccccgcgcagacgcgctggcctccgccgcggccgtgggcgacctgccgacatgccagcggctgctgctggaggagggctgccactgggacgcggcgaggctgtggcacgccgccgccggctacgggcacctgcatgtgtgcaaatggctggatgaaatgggcatggacaccgaatgggccatgtgcggcggtgatgtcttgagcttgggccgcggcggcgtgcccctcgcggtgcaggcggcggccgccaatggccaccgcgacgtactgcagtggctgcgggggatctggaagatacccgcggaagtcatcgcgggagcggccgcggaggggagccagatggggctgttctgggaacaggagcccgaggcggccggcgaccgtgtcggtcgccagctgctccttaaggtgctgcacggctgcccgctcagcgacctgcagcgcttctgcaaggcgcggaacctggaaagcgcgttgtctgacgcccccgacagcaccaagcacgatctgctgatgcgtgcggcgaccagccccacggccgactgggcggacaagtgcggctggctgctgtcgctgtggcgcaccccgccctcggaatgggcggagctagacgacgaaggcatcgaaggaaataagcttgtctacctttgggaacggctctggcaggccgtcgcgcggctgcagccggacacgttgctgccacgggtgcagtacctggtggctcgcgggctgcgcctgtcggagacggacgcgcttgtaccgcaccaggcagcggccgccgccggccacatggcaggggtggccatggcactgcgcgccgggccgccgctgctgcagcttcagggtccgtggggcagcggcaccgctgaggacccggcgcggcggggactgcggcgccacgtgtctagctttctctcctccgtcgcaattaaggcgggccaggcgtccgtccttccgttgtggggccgcgtcttcatgcctccgatcttaaaatgggccctgaagaatgtctgggacacgtatggcgacgtcgtggcttcccaaacggaggaccctttccaaggcctgcccgctgtgcgctaccttgtgatggaggaccaggtgtgcaagccagccaagggcgagcccggccccgactggtcggcgatcttccggcatgtggcgcgaagcggcgcggacttaccgctgctgcgcttcctacatgagcggcgtggcgcggccatcgacctggcggcggtagcgcgcggcggcggcgaggagcagctggagtgggcgctgggcgccctgcacgcagccggccagccatgtgacgagcctctggccgaggaccagtttatggcgattctggaggccggcaactgggcggcggcggactggctgctgcaccgcaggctggcaccagaacccctggagctgcagtttgacctgctgcagcacatcggagcgctcaaccggttggtcccggcgctgcgctggtttgtgagccgccacgaagggctgcgctggctgtcggacatggcagccgccgttaaggagaagtacagcgggggggcagcggagcaggaagacagcccggaggaagaatcggaggaagaatcggaggaagagtcggaggaagaatcggaggatagcgaggaggcgggtgaggaggcgggtgtgcaggaggacgcacaagagggcgcgcacgcggctgcaagcgcgggtgtgggcggcggtgctgctggagacaca

SEQ ID NO: 120 - protein, 639 aa, Chlamydomonas reinhardtii
NIPEPLLAEHPSWPGTAFVAHWGRPEPWRVLNRRQRHRLVCLAASSLHPPSLDAVLAHCGVLPRADALASAAAVGDLPTCQRLLLEEGCHWDAARLWHAAAGYGHLHVCKWLDEMGMDTEWAMCGGDVLSLGRGGVPLAVQAAAANGHRDVLQWLRGIWKIPAEVIAGAAAEGSQMGLFWEQEPEAAGDRVGRQLLLKVLHGCPLSDLQRFCKARNLESALSDAPDSTKHDLLMRAATSPTADWADKCGWLLSLWRTPPSEWAELDDEGIEGNKLVYLWERLWQAVARLQPDTLLPRVQYLVARGLRLSETDALVPHQAAAAAGHMAGVAMALRAGPPLLQLQGPWGSGTAEDPARRGLRRHVSSFLSSVAIKAGQASVLPLWGRVFMPPILKWALKNVWDTYGDVVASQTEDPFQGLPAVRYLVMEDQVCKPAKGEPGPDWSAIFRHVARSGADLPLLRFLHERRGAAIDLAAVARGGGEEQLEWALGALHAAGQPCDEPLAEDQFMAILEAGNWAAADWLLHRRLAPEPLELQFDLLQHIGALNRLVPALRWFVSRHEGLRWLSDMAAAVKEKYSGGAAEQEDSPEEESEEESEEESEEESEDSEEAGEEAGVQEDAQEGAHAAASAGVGGGAAGDT

SEQ ID NO: 121 - DNA, 1875 bp, Chlamydomonas reinhardtii
atgcctccggagtattggaccgattacatgtggctggtgatttgcggcgccctcgcctcattcgccatggcctggggtattggtgctaatgacgtcgccaactccttcgccacctccgtcggcgccaagaccatctcccttcgccaggcctgcattatcgccggtatcttcgagttcgctggtgccctgggcctgggcggcgaggtggcacgcaccatcgccggctccatcgcccgccccgccgcgttccaagacatgcccgagatgtttgcgtacggcatgctgtgcgccctgatctccgcctctacctggctttacattgccacgtacctgtccctggccgtgtccaccacccactccatcatcggcgccgtgctgggctttgctctggtgtggggcggttccggcgccgtggtgtggaacgaccgcaagaaggagttcccctactctaccggtctggtgcccgtcatctgctcctggttcgtgtcgcccatcactgctggcattgctgccggtgtcctctactttttcaaccgcctcatcgtgctgcgccgccagaactccaccaccctggccatctgggtgttcccgttgctggtgttcctgactgtgttcatcaacctcttcttcgttatctacaagggcgccagcaaggttgcgggctggaccagccacaaggccgcctgggtgtcggccgtggtggctgctggcctgtttgtgctggcttgcttccccggcacttggattctgcgccgccaggtcaccaaggacatggacgaggctgcccagaaggccgctgacgccgaggccaacgctggcaaggagaaggagggtgtggaggatgctgagcccacctccaaggccatgaagatcttcaactcgatcaagaaggccgccacccgtggcctggacgtggacatccatgcgaacgtcgagaccgaccgtgccgtccacgacctgcacgccgccgccgaggtgttctcccccgagaccgagcaggtgtacaagtacctgcaggtcttctccgcctgcgccgtgtctttcgcgcacggagccaacgatgtcgccaacgctgtgggccccttcgccggtatctaccacgtgtaccgcttctggaacgtcgcctccaacggcgagacccccatctgggtgcttgctatgggcggcgccggcattgtggtgggcctggctacctacggctacaacatcattgtgaccctgggcgtgggcctggccaagatgactcccgcccgtggctactgcgctgagctggccgccggcatcaccatctccgtcgcctccgtgtacggcctgcctgtgtccaccactcagattatcaccggtgccgaaatgggtgtgggcctggtcgagagcgtccgcaccggcgtgaactacaggctgctggccaagcaattcgccgcctgggtgttcaccctgatcattgccggcttcctgtctgccgccatcttcgctttcggcgcctacgctccgtcgctgaccatgggcaaggacatcttgaagtatgagacgtccatccgcaacctgaccaccgtcatctaccgctcgctcaacgcctccaacgtggcggtgaacaccagcttccctggtcagttcgaccccacgctgaaccgcaccatcaccaccaacacgcggaccctcaccaacatgttcaacagtaagaccattggttacatcgaccccacccagctggcgaccgagatgaacaacgcgtaccttacctacgtgaactactctgtagtcaccaccggcttcaaccgctccacccgcgcctacgtgcccgccaaccagccctacgccgccagcactatcgaggtcaagccctacgccgtggccgctcccgccggcgggcggtaa

SEQ ID NO: 122 - protein, 624 aa, Chlamydomonas reinhardtii
MPPEYWTDYMWLVICGALASFAMAWGIGANDVANSFATSVGAKTISLRQACIIAGIFEFAGALGLGGEVARTIAGSIARPAAFQDMPEMFAYGMLCALISASTWLYIATYLSLAVSTTHSIIGAVLGFALVWGGSGAVVWNDRKKEFPYSTGLVPVICSWFVSPITAGIAAGVLYFFNRLIVLRRQNSTTLAIWVFPLLVFLTVFINLFFVIYKGASKVAGWTSHKAAWVSAVVAAGLFVLACFPGTWILRRQVTKDMDEAAQKAADAEANAGKEKEGVEDAEPTSKAMKIFNSIKKAATRGLDVDIHANVETDRAVHDLHAAAEVFSPETEQVYKYLQVFSACAVSFAHGANDVANAVGPFAGIYHVYRFWNVASNGETPIWVLAMGGAGIVVGLATYGYNIIVTLGVGLAKMTPARGYCAELAAGITISVASVYGLPVSTTQIITGAEMGVGLVESVRTGVNYRLLAKQFAAWVFTLIIAGFLSAAIFAFGAYAPSLTMGKDILKYETSIRNLTTVIYRSLNASNVAVNTSFPGQFDPTLNRTITTNTRTLTNMFNSKTIGYIDPTQLATEMNNAYLTYVNYSVVTTGFNRSTRAYVPANQPYAASTIEVKPYAVAAPAGGR

SEQ ID NO: 123 - DNA, 1896 bp, artificial sequence
  codon optimized
catatgctcgagcctcctgagtattggactgattatatgtggctggtgatttgtggtgctctggcgtccttcgctatggcgtggggcatcggcgcgaacgatgtggccaactcgttcgcaacgagcgtcggcgccaagactatctccctgcgccaggcctgcatcatcgcgggcatcttcgagtttgcaggggccctgggcctcggcggcgaggtggcgcgcaccatcgccgggagcatcgcccgtcccgccgcgttccaggacatgcctgagatgttcgcatacgggatgctgtgcgccctcatcagcgcttcgacatggctgtacatcgcgacgtacctgtccctggccgtctcgaccactcactccatcattggcgcggtgctcggtttcgccctggtgtggggcggttcgggggcggtggtgtggaacgaccgcaagaaggagttcccctacagcaccggcctggtgcctgtgatttgcagctggttcgtgagccctattaccgcggggatcgccgcaggcgtgctgtacttctttaaccgcctcattgtgctgcgccggcagaactccactacgctggcgatctgggtgttcccgctgctggtcttcctcacggtgttcatcaacctgttctttgtgatctataagggcgcctcgaaggtggccggctggacttcgcacaaggcggcgtgggtgtcggcggtggtggccgctggcctgttcgtgctggcttgcttccctggcacgtggattctgcgccgtcaggtgacaaaggacatggacgaggcggcccagaaggctgccgacgccgaggcaaacgccggtaaggagaaggagggcgtggaggacgcggagcctaccagcaaggccatgaagattttcaactcgatcaagaaggccgccacgcgtggcctggacgtggatatccacgccaacgtcgagactgaccgcgccgtgcacgacctgcacgctgcggcggaggtgttcagcccggagactgagcaagtgtacaagtatctgcaagtgttctccgcctgcgccgtgagcttcgcccacggcgccaacgatgtcgcaaacgctgtgggcccattcgccggcatctaccacgtgtaccgcttctggaacgtcgcgagcaacggcgagacacccatctgggtcctggcgatgggtggcgctggcatcgtggtgggcctggcgacgtacggctacaacatcatcgtcacgctgggcgtgggtctggctaagatgactcccgcgcgtggctactgcgcggagctggcggctggcatcacgatctcggtggcgtcggtgtacgggctgcccgtgtccacgacccagattatcacaggcgcggagatgggcgtcggcctcgtggagagcgtccgcaccggcgtgaattaccgtctcctggccaagcaattcgcggcttgggtgttcacactgatcatcgcaggtttcctgtccgccgcgatttttgcctttggcgcctacgcgccctccctgacgatggggaaggacatcctgaagtacgagacttccatccggaacctgaccacggtgatctaccgcagcctgaacgcctccaacgtcgcggtgaacaccagctttcccggccagttcgatccgacgctgaaccgcacgatcaccaccaacactcgcacgctcaccaacatgtttaacagcaagaccatcggctacattgacccgacgcagctggcgaccgagatgaacaacgcctacctgacatacgtgaactactcggtggtgaccacgggcttcaaccgctcgacacgtgcctacgtgcctgccaaccagccttacgcggcttcgaccattgaggtgaagccctacgccgtggccgccccggctggcgggcggaccggttaaggatcc

SEQ ID NO: 124 - DNA, 1869 bp, artificial sequence
  codon optimized
cctcctgagtattggactgattatatgtggctggtgatttgtggtgctctggcgtccttcgctatggcgtggggcatcggcgcgaacgatgtggccaactcgttcgcaacgagcgtcggcgccaagactatctccctgcgccaggcctgcatcatcgcgggcatcttcgagtttgcaggggccctgggcctcggcggcgaggtggcgcgcaccatcgccgggagcatcgcccgtcccgccgcgttccaggacatgcctgagatgttcgcatacgggatgctgtgcgccctcatcagcgcttcgacatggctgtacatcgcgacgtacctgtccctggccgtctcgaccactcactccatcattggcgcggtgctcggtttcgccctggtgtggggcggttcgggggcggtggtgtggaacgaccgcaagaaggagttcccctacagcaccggcctggtgcctgtgatttgcagctggttcgtgagccctattaccgcggggatcgccgcaggcgtgctgtacttctttaaccgcctcattgtgctgcgccggcagaactccactacgctggcgatctgggtgttcccgctgctggtcttcctcacggtgttcatcaacctgttctttgtgatctataagggcgcctcgaaggtggccggctggacttcgcacaaggcggcgtgggtgtcggcggtggtggccgctggcctgttcgtgctggcttgcttccctggcacgtggattctgcgccgtcaggtgacaaaggacatggacgaggcggcccagaaggctgccgacgccgaggcaaacgccggtaaggagaaggagggcgtggaggacgcggagcctaccagcaaggccatgaagattttcaactcgatcaagaaggccgccacgcgtggcctggacgtggatatccacgccaacgtcgagactgaccgcgccgtgcacgacctgcacgctgcggcggaggtgttcagcccggagactgagcaagtgtacaagtatctgcaagtgttctccgcctgcgccgtgagcttcgcccacggcgccaacgatgtcgcaaacgctgtgggcccattcgccggcatctaccacgtgtaccgcttctggaacgtcgcgagcaacggcgagacacccatctgggtcctggcgatgggtggcgctggcatcgtggtgggcctggcgacgtacggctacaacatcatcgtcacgctgggcgtgggtctggctaagatgactcccgcgcgtggctactgcgcggagctggcggctggcatcacgatctcggtggcgtcggtgtacgggctgcccgtgtccacgacccagattatcacaggcgcggagatgggcgtcggcctcgtggagagcgtccgcaccggcgtgaattaccgtctcctggccaagcaattcgcggcttgggtgttcacactgatcatcgcaggtttcctgtccgccgcgatttttgcctttggcgcctacgcgccctccctgacgatggggaaggacatcctgaagtacgagacttccatccggaacctgaccacggtgatctaccgcagcctgaacgcctccaacgtcgcggtgaacaccagctttcccggccagttcgatccgacgctgaaccgcacgatcaccaccaacactcgcacgctcaccaacatgtttaacagcaagaccatcggctacattgacccgacgcagctggcgaccgagatgaacaacgcctacctgacatacgtgaactactcggtggtgaccacgggcttcaaccgctcgacacgtgcctacgtgcctgccaaccagccttacgcggcttcgaccattgaggtgaagccctacgccgtggccgccccggctggcgggcgg

SEQ ID NO: 125 - DNA, 1869 bp, Chlamydomonas reinhardtii
cctccggagtattggaccgattacatgtggctggtgatttgcggcgccctcgcctcattcgccatggcctggggtattggtgctaatgacgtcgccaactccttcgccacctccgtcggcgccaagaccatctcccttcgccaggcctgcattatcgccggtatcttcgagttcgctggtgccctgggcctgggcggcgaggtggcacgcaccatcgccggctccatcgcccgccccgccgcgttccaagacatgcccgagatgtttgcgtacggcatgctgtgcgccctgatctccgcctctacctggctttacattgccacgtacctgtccctggccgtgtccaccacccactccatcatcggcgccgtgctgggctttgctctggtgtggggcggttccggcgccgtggtgtggaacgaccgcaagaaggagttcccctactctaccggtctggtgcccgtcatctgctcctggttcgtgtcgcccatcactgctggcattgctgccggtgtcctctactttttcaaccgcctcatcgtgctgcgccgccagaactccaccaccctggccatctgggtgttcccgttgctggtgttcctgactgtgttcatcaacctcttcttcgttatctacaagggcgccagcaaggttgcgggctggaccagccacaaggccgcctgggtgtcggccgtggtggctgctggcctgtttgtgctggcttgcttccccggcacttggattctgcgccgccaggtcaccaaggacatggacgaggctgcccagaaggccgctgacgccgaggccaacgctggcaaggagaaggagggtgtggaggatgctgagcccacctccaaggccatgaagatcttcaactcgatcaagaaggccgccacccgtggcctggacgtggacatccatgcgaacgtcgagaccgaccgtgccgtccacgacctgcacgccgccgccgaggtgttctcccccgagaccgagcaggtgtacaagtacctgcaggtcttctccgcctgcgccgtgtctttcgcgcacggagccaacgatgtcgccaacgctgtgggccccttcgccggtatctaccacgtgtaccgcttctggaacgtcgcctccaacggcgagacccccatctgggtgcttgctatgggcggcgccggcattgtggtgggcctggctacctacggctacaacatcattgtgaccctgggcgtgggcctggccaagatgactcccgcccgtggctactgcgctgagctggccgccggcatcaccatctccgtcgcctccgtgtacggcctgcctgtgtccaccactcagattatcaccggtgccgaaatgggtgtgggcctggtcgagagcgtccgcaccggcgtgaactacaggctgctggccaagcaattcgccgcctgggtgttcaccctgatcattgccggcttcctgtctgccgccatcttcgctttcggcgcctacgctccgtcgctgaccatgggcaaggacatcttgaagtatgagacgtccatccgcaacctgaccaccgtcatctaccgctcgctcaacgcctccaacgtggcggtgaacaccagcttccctggtcagttcgaccccacgctgaaccgcaccatcaccaccaacacgcggaccctcaccaacatgttcaacagtaagaccattggttacatcgaccccacccagctggcgaccgagatgaacaacgcgtaccttacctacgtgaactactctgtagtcaccaccggcttcaaccgctccacccgcgcctacgtgcccgccaaccagccctacgccgccagcactatcgaggtcaagccctacgccgtggccgctcccgccggcgggcgg

SEQ ID NO: 126 - protein, 623 aa, Chlamydomonas reinhardtii
PPEYWTDYMWLVICGALASFAMAWGIGANDVANSFATSVGAKTISLRQACIIAGIFEFAGALGLGGEVARTIAGSIARPAAFQDMPEMFAYGMLCALISASTWLYIATYLSLAVSTTHSIIGAVLGFALVWGGSGAVVWNDRKKEFPYSTGLVPVICSWFVSPITAGIAAGVLYFFNRLIVLRRQNSTTLAIWVFPLLVFLTVFINLFFVIYKGASKVAGWTSHKAAWVSAVVAAGLFVLACFPGTWILRRQVTKDMDEAAQKAADAEANAGKEKEGVEDAEPTSKAMKIFNSIKKAATRGLDVDIHANVETDRAVHDLHAAAEVFSPETEQVYKYLQVFSACAVSFAHGANDVANAVGPFAGIYHVYRFWNVASNGETPIWVLAMGGAGIVVGLATYGYNIIVTLGVGLAKMTPARGYCAELAAGITISVASVYGLPVSTTQIITGAEMGVGLVESVRTGVNYRLLAKQFAAWVFTLIIAGFLSAAIFAFGAYAPSLTMGKDILKYETSIRNLTTVIYRSLNASNVAVNTSFPGQFDPTLNRTITTNTRTLTNMFNSKTIGYIDPTQLATEMNNAYLTYVNYSVVTTGFNRSTRAYVPANQPYAASTIEVKPYAVAAPAGGR

SEQ ID NO: 127 - DNA, 1416 bp, Chlamydomonas reinhardtii
atgaccacaccacaaccacctgctgcacagcaagggcggctggtgagcatttcgttccgcggaagaacacgacgatttgacacggacggagacggccgtctctgcttcaagcacgtcgcgaaagtgttcaagctcaacgccaacctcagtacagccgatgactggacgctcgatgacacgtcgctgcgactcgacgaccagggcctaacctgcagtgcactccgggacctgtttcctctccctgcgggcaccgcagaacagccactcgtgctgcagacgggtgatgatgccacaccaccaccctcaacctctgggcgggggacacccggggtccgcggagcaagtaatcagaaagcactggggctgccactgccggccccgacgccgggccgggccgtgggcctgggtccacgtcagcagggcgcgctgctggacctgtctcggccgctgccgcccgacagcgagttgctgaggcacccgctcgggcatggctctttcttcttccccgcctccgccgccagccacctgcccatgctcatggcctgccacctgccgccgccgctgcagcgcctgctgcgcgccttccgactgggggcccagtgtgccatcacggcgcgggccatggcgctgtatgcggccaggacggcggcggccggcacggacaccggcgcaggggcaggcgcaggtccggggcagctggagtacttcgcggacgccaggctggaggtagctatcaccgaggcgcgcagctacaacccgccgcaggaccggtactgcgcctactcccaatccgtgcggctgacgggtaagaagcgcaagcccgccgcctcaccccggcccgcgcccgccacgccctcggctgccggcgctcgcgccaccaccccgctttcagcagcgcgcagcgctgccactgcggccgcctccgcctccgcaaccactatcggtggcgcagccggccccgcccccgctaccaccactcacggcaccaccgcagctgctgatgtggacatggacgcctcgccgttgggctgggcgttcgtgggcggcggcacagtgcactgcccgggcggcgtggcgtgccggcgcgcggggcggcatgagaactggctgctgtcggacgcggccgaccgccacatcaccttcctgcccgtgctgtacgacagcgaggagcacagcgccgtactgctgccgctggcggcggacgcgcgggcgggcggcccggtggggcgggccggcgacggaggccaggagggcgcggccgcgggtgtggtgtggttcaagtacgcgtatgagagccgcggggggcggcgcggccacaacttttgggactggggcctgggtgagcacttggagcaggcggtgcgggacgtggcgggagaggaggcactgcacgcggcgactggcggcattgcgtaa

SEQ ID NO: 128 - protein, 471 aa, Chlamydomonas reinhardtii
MTTPQPPAAQQGRLVSISFRGRTRRFDTDGDGRLCFKHVAKVFKLNANLSTADDWTLDDTSLRLDDQGLTCSALRDLFPLPAGTAEQPLVLQTGDDATPPPSTSGRGTPGVRGASNQKALGLPLPAPTPGRAVGLGPRQQGALLDLSRPLPPDSELLRHPLGHGSFFFPASAASHLPMLMACHLPPPLQRLLRAFRLGAQCAITARAMALYAARTAAAGTDTGAGAGAGPGQLEYFADARLEVAITEARSYNPPQDRYCAYSQSVRLTGKKRKPAASPRPAPATPSAAGARATTPLSAARSAATAAASASATTIGGAAGPAPATTTHGTTAAADVDMDASPLGWAFVGGGTVHCPGGVACRRAGRHENWLLSDAADRHITFLPVLYDSEEHSAVLLPLAADARAGGPVGRAGDGGQEGAAAGVVWFKYAYESRGGRRGHNFWDWGLGEHLEQAVRDVAGEEALHAATGGIA

SEQ ID NO: 129 - DNA, 1431 bp, artificial sequence
  codon optimized
catatgctcgagactacacctcagccaccggcagcacaacagggtcgtctggtgtccatcagcttccgtgggcggacccggcgtttcgacactgacggcgatgggcgcctgtgcttcaagcatgtggcgaaggtgttcaagctgaacgccaacctctcgaccgccgacgactggacgctcgacgatacgagcctgcgcctggacgaccagggcctgacctgctcggctctgcgcgacctcttcccgctgcctgctggcacagctgagcagccgctggtcctccagaccggcgacgacgcgacaccgcctccaagcacctccggccgtggcactccgggcgtccgcggtgccagcaaccagaaggccctgggcctgccactgccagcgccgacaccgggccgcgcggtcggcctcggcccacggcagcagggggccctgctcgatctgtcccgccctctgcctccggactcggagctgctgcggcaccccctgggccacggctcgttcttcttccctgcctcggccgcttcgcacctgccgatgctgatggcgtgccacctgccccctccgctgcaacgcctcctgcgcgccttccgcctgggggcccagtgcgcgatcaccgcacgtgcgatggccctgtatgccgctcgcacggcggctgcgggcacggacaccggcgctggcgcaggtgcgggccctggtcagctggagtacttcgcggacgcccggctggaggtcgccattacggaggcccgtagctataacccgccccaggaccgctactgcgcctactcccagtccgtgcggctgacggggaagaagcgcaagccggcagcgagccctcgccccgctcccgccactccgtccgcggctggcgcacgcgcaacaacccctctgtccgcggcacggagcgccgccacggccgcagcgagcgcctcggccactaccattggcggcgctgccggccctgcccccgccaccaccacgcacggcacgacggccgcggcggacgtggacatggatgcctccccgctgggctgggcctttgtgggtggcggcaccgtgcactgtcctggtggcgtggcctgccggcgggcgggtcggcacgagaactggctgctgtcggacgccgcagaccgtcacatcacgttcctgcccgtgctgtacgactcggaggagcattcggccgtgctgctgccgctggcggcagacgcgcgcgccggtggccccgtgggtcgcgcaggcgacgggggccaggagggcgcagctgcgggggtcgtgtggtttaagtacgcctacgagagccgcggtggtcgtcgtggccataacttctgggactggggcctgggcgagcacctggagcaagcggtgcgcgacgtggctggcgaggaggcgctccacgcggctaccggcggcattgcgtagggatcc

SEQ ID NO: 130 - DNA, 1410 bp, artificial sequence
  codon optimized
actacacctcagccaccggcagcacaacagggtcgtctggtgtccatcagcttccgtgggcggacccggcgtttcgacactgacggcgatgggcgcctgtgcttcaagcatgtggcgaaggtgttcaagctgaacgccaacctctcgaccgccgacgactggacgctcgacgatacgagcctgcgcctggacgaccagggcctgacctgctcggctctgcgcgacctcttcccgctgcctgctggcacagctgagcagccgctggtcctccagaccggcgacgacgcgacaccgcctccaagcacctccggccgtggcactccgggcgtccgcggtgccagcaaccagaaggccctgggcctgccactgccagcgccgacaccgggccgcgcggtcggcctcggcccacggcagcagggggccctgctcgatctgtcccgccctctgcctccggactcggagctgctgcggcaccccctgggccacggctcgttcttcttccctgcctcggccgcttcgcacctgccgatgctgatggcgtgccacctgccccctccgctgcaacgcctcctgcgcgccttccgcctgggggcccagtgcgcgatcaccgcacgtgcgatggccctgtatgccgctcgcacggcggctgcgggcacggacaccggcgctggcgcaggtgcgggccctggtcagctggagtacttcgcggacgcccggctggaggtcgccattacggaggcccgtagctataacccgccccaggaccgctactgcgcctactcccagtccgtgcggctgacggggaagaagcgcaagccggcagcgagccctcgccccgctcccgccactccgtccgcggctggcgcacgcgcaacaacccctctgtccgcggcacggagcgccgccacggccgcagcgagcgcctcggccactaccattggcggcgctgccggccctgcccccgccaccaccacgcacggcacgacggccgcggcggacgtggacatggatgcctccccgctgggctgggcctttgtgggtggcggcaccgtgcactgtcctggtggcgtggcctgccggcgggcgggtcggcacgagaactggctgctgtcggacgccgcagaccgtcacatcacgttcctgcccgtgctgtacgactcggaggagcattcggccgtgctgctgccgctggcggcagacgcgcgcgccggtggccccgtgggtcgcgcaggcgacgggggccaggagggcgcagctgcgggggtcgtgtggtttaagtacgcctacgagagccgcggtggtcgtcgtggccataacttctgggactggggcctgggcgagcacctggagcaagcggtgcgcgacgtggctggcgaggaggcgctccacgcggctaccggcggcattgcg

SEQ ID NO: 131 - DNA, 1410 bp, Chlamydomonas reinhardtii
accacaccacaaccacctgctgcacagcaagggcggctggtgagcatttcgttccgcggaagaacacgacgatttgacacggacggagacggccgtctctgcttcaagcacgtcgcgaaagtgttcaagctcaacgccaacctcagtacagccgatgactggacgctcgatgacacgtcgctgcgactcgacgaccagggcctaacctgcagtgcactccgggacctgtttcctctccctgcgggcaccgcagaacagccactcgtgctgcagacgggtgatgatgccacaccaccaccctcaacctctgggcgggggacacccggggtccgcggagcaagtaatcagaaagcactggggctgccactgccggccccgacgccgggccgggccgtgggcctgggtccacgtcagcagggcgcgctgctggacctgtctcggccgctgccgcccgacagcgagttgctgaggcacccgctcgggcatggctctttcttcttccccgcctccgccgccagccacctgcccatgctcatggcctgccacctgccgccgccgctgcagcgcctgctgcgcgccttccgactgggggcccagtgtgccatcacggcgcgggccatggcgctgtatgcggccaggacggcggcggccggcacggacaccggcgcaggggcaggcgcaggtccggggcagctggagtacttcgcggacgccaggctggaggtagctatcaccgaggcgcgcagctacaacccgccgcaggaccggtactgcgcctactcccaatccgtgcggctgacgggtaagaagcgcaagcccgccgcctcaccccggcccgcgcccgccacgccctcggctgccggcgctcgcgccaccaccccgctttcagcagcgcgcagcgctgccactgcggccgcctccgcctccgcaaccactatcggtggcgcagccggccccgcccccgctaccaccactcacggcaccaccgcagctgctgatgtggacatggacgcctcgccgttgggctgggcgttcgtgggcggcggcacagtgcactgcccgggcggcgtggcgtgccggcgcgcggggcggcatgagaactggctgctgtcggacgcggccgaccgccacatcaccttcctgcccgtgctgtacgacagcgaggagcacagcgccgtactgctgccgctggcggcggacgcgcgggcgggcggcccggtggggcgggccggcgacggaggccaggagggcgcggccgcgggtgtggtgtggttcaagtacgcgtatgagagccgcggggggcggcgcggccacaacttttgggactggggcctgggtgagcacttggagcaggcggtgcgggacgtggcgggagaggaggcactgcacgcggcgactggcggcattgcg

SEQ ID NO: 132 - protein, 470 aa, Chlamydomonas reinhardtii
TTPQPPAAQQGRLVSISFRGRTRRFDTDGDGRLCFKHVAKVFKLNANLSTADDWTLDDTSLRLDDQGLTCSALRDLFPLPAGTAEQPLVLQTGDDATPPPSTSGRGTPGVRGASNQKALGLPLPAPTPGRAVGLGPRQQGALLDLSRPLPPDSELLRHPLGHGSFFFPASAASHLPMLMACHLPPPLQRLLRAFRLGAQCAITARAMALYAARTAAAGTDTGAGAGAGPGQLEYFADARLEVAITEARSYNPPQDRYCAYSQSVRLTGKKRKPAASPRPAPATPSAAGARATTPLSAARSAATAAASASATTIGGAAGPAPATTTHGTTAAADVDMDASPLGWAFVGGGTVHCPGGVACRRAGRHENWLLSDAADRHITFLPVLYDSEEHSAVLLPLAADARAGGPVGRAGDGGQEGAAAGVVWFKYAYESRGGRRGHNFWDWGLGEHLEQAVRDVAGEEALHAATGGIA

SEQ ID NO: 133 - DNA, 741 bp, Chlamydomonas reinhardtii
atgggctccaacaaccagctcaagctcttggacctggggctgcagccgctactgccgcagctactgccggcgccagcttcagcagcagcagcagcagcagcagcagcagccgggccgccgccgcgcagcctgcccgcagacctgggtgcgctgctggaggacgcgcagcagcccggcggcggcgggactgcagacctcacgcttcgtgtgggcgagcggcgcttccactgccaccgtgccatcctgtccgcgcgctgcgactacttcaagcagcgcctggcggcgagcggcggcttcgcggacgcgcgcgcggcggagctggagctgccggacgcggacgccgacaccttcgcgctgctgctgcgatggctgtacacgggcgacacggacgttccgctggagcaggcgcgcggtgtggctgagctggcggaccggctgctgctgcctgagctttgcgcccgtgcattggacgtggtggcggcatccgtggacgcggcggccatcgtggacagcctgctgtgggccgcgggctgctgcgaggcgcgtggcggcggcggcggctttggcggcctgctggcgcggctgaaggagtggtacgtgtcgcaccacaaggccgtggcggcggaggcgggagtcagtcgcgccaggctggctgcggaggcgccgctgctgatggtggagctgatggacgccgtgctgagtcagccggacgggggagaccggaagcggcagcgcgcgtag

SEQ ID NO: 134 - protein, 246 aa, Chlamydomonas reinhardtii
MGSNNQLKLLDLGLQPLLPQLLPAPASAAAAAAAAAAGPPPRSLPADLGALLEDAQQPGGGGTADLTLRVGERRFHCHRAILSARCDYFKQRLAASGGFADARAAELELPDADADTFALLLRWLYTGDTDVPLEQARGVAELADRLLLPELCARALDVVAASVDAAAIVDSLLWAAGCCEARGGGGGFGGLLARLKEWYVSHHKAVAAEAGVSRARLAAEAPLLMVELMDAVLSQPDGGDRKRQRA

SEQ ID NO: 135 - DNA, 762 bp, artificial sequence
  codon optimized
catatgctcgagggctcgaataatcaactcaagctcctggatctgggtctgcaaccactgctgccccaactgctccccgcacctgcgtcggccgctgccgccgctgcggctgctgcggcgggcccccccccgcgttccctgcccgccgacctgggcgccctgctggaggacgcccagcagcctggcggtggcggcaccgctgacctgactctgcgggtgggcgagcgccggttccactgccaccgcgccatcctgtcggcccgttgcgactacttcaagcagcggctcgcggccagcggcgggttcgccgacgcccgtgccgcggagctggagctgccggacgcggacgccgacacgttcgcgctcctgctccgttggctctacaccggcgacaccgacgtgcccctggagcaggcacgcggcgtggccgagctggctgaccgcctcctgctgcccgagctgtgcgcccgtgccctggacgtggtggctgccagcgtggacgcggctgccatcgtggacagcctcctgtgggcggctgggtgctgcgaggcccgtgggggcggcggcggcttcggtggcctgctggcccgcctcaaggagtggtacgtgagccaccacaaggcggtggctgccgaggcgggcgtgtcgcgtgcgcgcctggccgccgaggcacccctgctgatggtggagctgatggacgccgtgctgtcgcagccggacggcggtgaccgtaagcgccagcgcgcgaccggttgaggatcc

SEQ ID NO: 136 - DNA, 735 bp, artificial sequence
  codon optimized
ggctcgaataatcaactcaagctcctggatctgggtctgcaaccactgctgccccaactgctccccgcacctgcgtcggccgctgccgccgctgcggctgctgcggcgggcccccccccgcgttccctgcccgccgacctgggcgccctgctggaggacgcccagcagcctggcggtggcggcaccgctgacctgactctgcgggtgggcgagcgccggttccactgccaccgcgccatcctgtcggcccgttgcgactacttcaagcagcggctcgcggccagcggcgggttcgccgacgcccgtgccgcggagctggagctgccggacgcggacgccgacacgttcgcgctcctgctccgttggctctacaccggcgacaccgacgtgcccctggagcaggcacgcggcgtggccgagctggctgaccgcctcctgctgcccgagctgtgcgcccgtgccctggacgtggtggctgccagcgtggacgcggctgccatcgtggacagcctcctgtgggcggctgggtgctgcgaggcccgtgggggcggcggcggcttcggtggcctgctggcccgcctcaaggagtggtacgtgagccaccacaaggcggtggctgccgaggcgggcgtgtcgcgtgcgcgcctggccgccgaggcacccctgctgatggtggagctgatggacgccgtgctgtcgcagccggacggcggtgaccgtaagcgccagcgcgcg

SEQ ID NO: 137 - DNA, 735 bp, Chlamydomonas reinhardtii
ggctccaacaaccagctcaagctcttggacctggggctgcagccgctactgccgcagctactgccggcgccagcttcagcagcagcagcagcagcagcagcagcagccgggccgccgccgcgcagcctgcccgcagacctgggtgcgctgctggaggacgcgcagcagcccggcggcggcgggactgcagacctcacgcttcgtgtgggcgagcggcgcttccactgccaccgtgccatcctgtccgcgcgctgcgactacttcaagcagcgcctggcggcgagcggcggcttcgcggacgcgcgcgcggcggagctggagctgccggacgcggacgccgacaccttcgcgctgctgctgcgatggctgtacacgggcgacacggacgttccgctggagcaggcgcgcggtgtggctgagctggcggaccggctgctgctgcctgagctttgcgcccgtgcattggacgtggtggcggcatccgtggacgcggcggccatcgtggacagcctgctgtgggccgcgggctgctgcgaggcgcgtggcggcggcggcggctttggcggcctgctggcgcggctgaaggagtggtacgtgtcgcaccacaaggccgtggcggcggaggcgggagtcagtcgcgccaggctggctgcggaggcgccgctgctgatggtggagctgatggacgccgtgctgagtcagccggacgggggagaccggaagcggcagcgcgcg

SEQ ID NO: 138 - protein, 245 aa, Chlamydomonas reinhardtii
GSNNQLKLLDLGLQPLLPQLLPAPASAAAAAAAAAAGPPPRSLPADLGALLEDAQQPGGGGTADLTLRVGERRFHCHRAILSARCDYFKQRLAASGGFADARAAELELPDADADTFALLLRWLYTGDTDVPLEQARGVAELADRLLLPELCARALDVVAASVDAAAIVDSLLWAAGCCEARGGGGGFGGLLARLKEWYVSHHKAVAAEAGVSRARLAAEAPLLMVELMDAVLSQPDGGDRKRQRA

SEQ ID NO: 139 - DNA, 1287 bp, Chlamydomonas reinhardtii
atgcatgccctgttcatcccctgccatgccacgccaccacctcccccacgccattccgcagatgattcccaagctcctccggattacggagcccagatggatcgggagcaccgacccaagcacaagctccgcgcctatctggtgtccggtctctgcaagaccagcagccagaaacaccccagcagcacgcctagtgatactgactgcattcttcgaagcgacaaggtcaatgccaaggccgaccaaaaggggtgtgtgaacccagatgcgcctgggcaccggtgcttcggccgcctactacatgccactgccctcttcggccgcgcgccgtcatccccaccattaccagcgcgcgccaaggccatctcgcgctcggtgatcatcatcatcgccatcctgccgaccctgtgcgccgccgcgctgtcggttgtctccactctgctcacggacgcgggcgcctgcaacctgacctcctccaccagcctgcgcatcaccctggccgccgccacctacaaccccggcgacgccatcactgtcgcggactcccagaccacgctgtatgccggcactgggctgagcaccgtgttcgacagcaccggcacccagatcaccgccgacctgaccggcgaccagtgcgcctccattctggaggtcaaggccggcaccaccgccaagaagtttgcctcctgcgccgtcagtggtgtcagctccggccgcggcactgtgctcacggtcaagctgctgggcaacaccacggacgtgtacgcctcgggcgacaccttcaacttcaaggacaccaacgccctgctgctggccggcaccaccaacaccgctcccgcctacaaggcgctggccaccgccgccaccatccgcatcacactgccctccgccagcggctttgtggtcagcggcgccgcctccaccaccatcgccgccgccaactgcgacttcctagtgttcaaccccgtgcgcaccgccaagaccgacaccgcctgcaacatcaccggctccacgcttaccatcaccctcaacaccgccattaccggcaccaccaccgtcaacatcgtgacgagccagaccaagctgctggtggccggcaccggcacctccggcccggccttcgtgccggccggctcccctatcgccatcagccccggcttcctgacctcacccgctgtggcgcgctcgctgacgcagctggatggttgtgtcgttgcccttcacctccaacgtgggcgccagcgcctcttggacccgcacccaggcgcagtgcaacagcgtcgttga

SEQ ID NO: 140 - protein, 428 aa, Chlamydomonas reinhardtii
MHALFIPCHATPPPPPRHSADDSQAPPDYGAQMDREHRPKHKLRAYLVSGLCKTSSQKHPSSTPSDTDCILRSDKVNAKADQKGCVNPDAPGHRCFGRLLHATALFGRAPSSPPLPARAKAISRSVIIIIAILPTLCAAALSVVSTLLTDAGACNLTSSTSLRITLAAATYNPGDAITVADSQTTLYAGTGLSTVFDSTGTQITADLTGDQCASILEVKAGTTAKKFASCAVSGVSSGRGTVLTVKLLGNTTDVYASGDTFNFKDTNALLLAGTTNTAPAYKALATAATIRITLPSASGFVVSGAASTTIAAANCDFLVFNPVRTAKTDTACNITGSTLTITLNTAITGTTTVNIVTSQTKLLVAGTGTSGPAFVPAGSPIAISPGFLTSPAVARSLTQLDGCVVALHLQRGRQRLLDPHPGAVQQRR

SEQ ID NO: 141 - DNA, 1302 bp, artificial sequence
  codon optimized
catatgctcgagcatgctctgtttattccttgtcatgctacacctccaccacctcctcgccacagcgcggacgactcgcaggcgcctcccgactacggcgcgcagatggaccgcgagcaccgccccaagcacaagctccgggcctacctggtgtcgggcctgtgcaagaccagcagccagaagcacccatcgtccacaccgagcgacacggactgcatcctgcgctccgacaaggtgaacgctaaggctgaccagaagggctgcgtgaacccagatgcaccaggccaccgctgtttcggtcgcctgctccacgcgaccgcgctctttggccgcgctccgtcgtcgccgccactgccagcccgcgctaaggctatcagccgctccgtgattatcatcatcgccatcctgccgaccctgtgcgcggcagcgctgagcgtggtgtccacgctgctgacggatgctggcgcgtgtaacctgaccagctccacgagcctgcgcattacgctggcggctgccacgtacaaccccggcgacgcaatcactgtggccgacagccagaccacgctgtacgcgggcacaggcctgagcaccgtcttcgactcgactggcacgcagattacggcggacctcactggcgaccagtgcgcgtcgatcctggaggtgaaggccggcaccacggcaaagaagttcgcctcctgtgccgtgagcggcgtgtccagcggtcgcggcacggtgctgacggtgaagctgctcggcaacacgaccgatgtgtacgcctcgggcgacacattcaactttaaggacacgaacgcgctgctcctggccgggaccaccaacactgccccagcctacaaggcgctggcaaccgcggctacgatccgcattaccctgccctcggcttcgggctttgtggtgtcgggcgccgcaagcactacgattgccgctgcgaactgcgacttcctggtcttcaaccctgtccgtaccgcaaagacagatacggcctgcaacatcacgggctccacgctgaccatcaccctgaacaccgccatcaccggcacgacaacggtcaacattgtgacgagccagacgaagctgctggtggccggcacgggcaccagcggtccagccttcgtgccggcaggctcgccaatcgcgatctccccagggttcctgactagcccagccgtggcccggtcgctgacccagctggacggctgcgtggtggccctgcacctccagcgtggccgtcagcggctgctggaccctcacccaggcgccgtgcagcagcgccgctaaggatcc

SEQ ID NO: 142 - DNA, 1281 bp, artificial sequence
  codon optimized
catgctctgtttattccttgtcatgctacacctccaccacctcctcgccacagcgcggacgactcgcaggcgcctcccgactacggcgcgcagatggaccgcgagcaccgccccaagcacaagctccgggcctacctggtgtcgggcctgtgcaagaccagcagccagaagcacccatcgtccacaccgagcgacacggactgcatcctgcgctccgacaaggtgaacgctaaggctgaccagaagggctgcgtgaacccagatgcaccaggccaccgctgtttcggtcgcctgctccacgcgaccgcgctctttggccgcgctccgtcgtcgccgccactgccagcccgcgctaaggctatcagccgctccgtgattatcatcatcgccatcctgccgaccctgtgcgcggcagcgctgagcgtggtgtccacgctgctgacggatgctggcgcgtgtaacctgaccagctccacgagcctgcgcattacgctggcggctgccacgtacaaccccggcgacgcaatcactgtggccgacagccagaccacgctgtacgcgggcacaggcctgagcaccgtcttcgactcgactggcacgcagattacggcggacctcactggcgaccagtgcgcgtcgatcctggaggtgaaggccggcaccacggcaaagaagttcgcctcctgtgccgtgagcggcgtgtccagcggtcgcggcacggtgctgacggtgaagctgctcggcaacacgaccgatgtgtacgcctcgggcgacacattcaactttaaggacacgaacgcgctgctcctggccgggaccaccaacactgccccagcctacaaggcgctggcaaccgcggctacgatccgcattaccctgccctcggcttcgggctttgtggtgtcgggcgccgcaagcactacgattgccgctgcgaactgcgacttcctggtcttcaaccctgtccgtaccgcaaagacagatacggcctgcaacatcacgggctccacgctgaccatcaccctgaacaccgccatcaccggcacgacaacggtcaacattgtgacgagccagacgaagctgctggtggccggcacgggcaccagcggtccagccttcgtgccggcaggctcgccaatcgcgatctccccagggttcctgactagcccagccgtggcccggtcgctgacccagctggacggctgcgtggtggccctgcacctccagcgtggccgtcagcggctgctggaccctcacccaggcgccgtgcagcagcgccgc

SEQ ID NO: 143 - DNA, 1281 bp, Chlamydomonas reinhardtii
catgccctgttcatcccctgccatgccacgccaccacctcccccacgccattccgcagatgattcccaagctcctccggattacggagcccagatggatcgggagcaccgacccaagcacaagctccgcgcctatctggtgtccggtctctgcaagaccagcagccagaaacaccccagcagcacgcctagtgatactgactgcattcttcgaagcgacaaggtcaatgccaaggccgaccaaaaggggtgtgtgaacccagatgcgcctgggcaccggtgcttcggccgcctactacatgccactgccctcttcggccgcgcgccgtcatccccaccattaccagcgcgcgccaaggccatctcgcgctcggtgatcatcatcatcgccatcctgccgaccctgtgcgccgccgcgctgtcggttgtctccactctgctcacggacgcgggcgcctgcaacctgacctcctccaccagcctgcgcatcaccctggccgccgccacctacaaccccggcgacgccatcactgtcgcggactcccagaccacgctgtatgccggcactgggctgagcaccgtgttcgacagcaccggcacccagatcaccgccgacctgaccggcgaccagtgcgcctccattctggaggtcaaggccggcaccaccgccaagaagtttgcctcctgcgccgtcagtggtgtcagctccggccgcggcactgtgctcacggtcaagctgctgggcaacaccacggacgtgtacgcctcgggcgacaccttcaacttcaaggacaccaacgccctgctgctggccggcaccaccaacaccgctcccgcctacaaggcgctggccaccgccgccaccatccgcatcacactgccctccgccagcggctttgtggtcagcggcgccgcctccaccaccatcgccgccgccaactgcgacttcctagtgttcaaccccgtgcgcaccgccaagaccgacaccgcctgcaacatcaccggctccacgcttaccatcaccctcaacaccgccattaccggcaccaccaccgtcaacatcgtgacgagccagaccaagctgctggtggccggcaccggcacctccggcccggccttcgtgccggccggctcccctatcgccatcagccccggcttcctgacctcacccgctgtggcgcgctcgctgacgcagctggatggttgtgtcgttgcccttcacctccaacgtgggcgccagcgcctcttggacccgcacccaggcgcagtgcaacagcgtcgt

SEQ ID NO: 144 - protein, 427 aa, Chlamydomonas reinhardtii
HALFIPCHATPPPPPRHSADDSQAPPDYGAQMDREHRPKHKLRAYLVSGLCKTSSQKHPSSTPSDTDCILRSDKVNAKADQKGCVNPDAPGHRCFGRLLHATALFGRAPSSPPLPARAKAISRSVIIIIAILPTLCAAALSVVSTLLTDAGACNLTSSTSLRITLAAATYNPGDAITVADSQTTLYAGTGLSTVFDSTGTQITADLTGDQCASILEVKAGTTAKKFASCAVSGVSSGRGTVLTVKLLGNTTDVYASGDTFNFKDTNALLLAGTTNTAPAYKALATAATIRITLPSASGFVVSGAASTTIAAANCDFLVFNPVRTAKTDTACNITGSTLTITLNTAITGTTTVNIVTSQTKLLVAGTGTSGPAFVPAGSPIAISPGFLTSPAVARSLTQLDGCVVALHLQRGRQRLLDPHPGAVQQRR

SEQ ID NO: 145 - DNA, 417 bp, Chlamydomonas reinhardtii
atgccactcgcaatcaccggcagcccgccttaccttgcatttacccgccctgcctgccacacctgcgagcacatctacgaccccaccacggatcgccctgatgacgatggcgtcatccccgcctgtcctgtgcccgactcttgcccggccgacgccgtgatgacggccgtcggcatccccgacctggcccgcagcaagtgcaagcccaacaccagctgcctgcagaagccgcccgagagccactactccaccttccccctggccaccttcccccggccaccgccaacatgctgtactccctgggctaccacgccaagtaagcccaccggggcccaagcgggcggcatgtggctggcgggcacctatacttcgaggcagccaacccgccggtgccaggaaacgcctgctacacgatga

SEQ ID NO: 146 - protein, 138 aa, Chlamydomonas reinhardtii
MPLAITGSPPYLAFTRPACHTCEHIYDPTTDRPDDDGVIPACPVPDSCPADAVMTAVGIPDLARSKCKPNTSCLQKPPESHYSTFPLATFPRPPPTCCTPWATTPSKPTGAQAGGMWLAGTYTSRQPTRRCQETPATR

SEQ ID NO: 147 - DNA, 432 bp, artificial sequence
  codon optimized
catatgctcgagcctctggctattacaggctccccaccttatctcgcttttactcgccccgcatgtcatacttgcgagcacatctacgaccccaccaccgaccgtccggatgacgacggcgtgatccccgcctgcccggtgccagactcctgccccgcggacgccgtgatgacggcggtgggcatcccggacctggctcggagcaagtgcaagccaaataccagctgcctgcaaaagccgcccgagtcgcattactcgacgttcccgctggccaccttcccgcgtccccctccgacctgctgcacgccctgggccactactccaagcaagcccacgggcgcgcaggctggcggcatgtggctggcgggcacctacaccagccgccaacccacccgtcggtgccaggagacgccagccacccgctaaggatcc

SEQ ID NO: 148 - DNA, 411 bp, artificial sequence
  codon optimized
cctctggctattacaggctccccaccttatctcgcttttactcgccccgcatgtcatacttgcgagcacatctacgaccccaccaccgaccgtccggatgacgacggcgtgatccccgcctgcccggtgccagactcctgccccgcggacgccgtgatgacggcggtgggcatcccggacctggctcggagcaagtgcaagccaaataccagctgcctgcaaaagccgcccgagtcgcattactcgacgttcccgctggccaccttcccgcgtccccctccgacctgctgcacgccctgggccactactccaagcaagcccacgggcgcgcaggctggcggcatgtggctggcgggcacctacaccagccgccaacccacccgtcggtgccaggagacgccagccacccgc

SEQ ID NO: 149 - DNA, 411 bp, Chlamydomonas reinhardtii
ccactcgcaatcaccggcagcccgccttaccttgcatttacccgccctgcctgccacacctgcgagcacatctacgaccccaccacggatcgccctgatgacgatggcgtcatccccgcctgtcctgtgcccgactcttgcccggccgacgccgtgatgacggccgtcggcatccccgacctggcccgcagcaagtgcaagcccaacaccagctgcctgcagaagccgcccgagagccactactccaccttccccctggccaccttcccccggccaccgccaacatgctgtactccctgggctaccacgccaagtaagcccaccggggcccaagcgggcggcatgtggctggcgggcacctatacttcgaggcagccaacccgccggtgccaggaaacgcctgctacacga

SEQ ID NO: 150 - protein, 137 aa, Chlamydomonas reinhardtii
PLAITGSPPYLAFTRPACHTCEHIYDPTTDRPDDDGVIPACPVPDSCPADAVMTAVGIPDLARSKCKPNTSCLQKPPESHYSTFPLATFPRPPPTCCTPWATTPSKPTGAQAGGMWLAGTYTSRQPTRRCQETPATR

SEQ ID NO: 151 - DNA, 279 bp, Chlamydomonas reinhardtii
atgacggaaaatgaattcgtcgtacggttctcgcatcgcaggtatccggcacgcccggaccagccgccgctgcggcagggctcctttggcgtgggcggcgccggtggcggcgaggagttcgggctgggggcggaggagcgtgtggaggtgcttgaggacgcagacgacgccggcagtgtggtggcggtgctgactgtgtcgctggtggcggaggcggccctggccacactgagggagaccatgaggcaccgcggcggcggagggggcgtgccgtactga

SEQ ID NO: 152 - protein, 92 aa, Chlamydomonas reinhardtii
MTENEFVVRFSHRRYPARPDQPPLRQGSFGVGGAGGGEEFGLGAEERVEVLEDADDAGSVVAVLTVSLVAEAALATLRETMRHRGGGGGVPY

SEQ ID NO: 153 - DNA, 288 bp, artificial sequence
  codon optimized
ctcgagactgagaatgagtttgtggtgcgttttagccatcgtcgttatcctgcgcgtcccgaccagcccccgctgcgccagggctcctttggcgtggggggcgcaggtggcggtgaggagttcggcctgggcgcggaggagcgtgtggaggtgctggaggacgcagacgacgccggtagcgtcgtggccgtgctgaccgtgtccctcgtggcggaggccgctctcgcgacgctgcgcgagactatgcgccatcgtggtggcggcgggggtgtgccgtactagggatcc

SEQ ID NO: 154 - DNA, 273 bp, artificial sequence
  codon optimized
actgagaatgagtttgtggtgcgttttagccatcgtcgttatcctgcgcgtcccgaccagcccccgctgcgccagggctcctttggcgtggggggcgcaggtggcggtgaggagttcggcctgggcgcggaggagcgtgtggaggtgctggaggacgcagacgacgccggtagcgtcgtggccgtgctgaccgtgtccctcgtggcggaggccgctctcgcgacgctgcgcgagactatgcgccatcgtggtggcggcgggggtgtgccgtac

SEQ ID NO: 155 - DNA, 273 bp, Chlamydomonas reinhardtii
acggaaaatgaattcgtcgtacggttctcgcatcgcaggtatccggcacgcccggaccagccgccgctgcggcagggctcctttggcgtgggcggcgccggtggcggcgaggagttcgggctgggggcggaggagcgtgtggaggtgcttgaggacgcagacgacgccggcagtgtggtggcggtgctgactgtgtcgctggtggcggaggcggccctggccacactgagggagaccatgaggcaccgcggcggcggagggggcgtgccgtac

SEQ ID NO: 156 - protein, 91 aa, Chlamydomonas reinhardtii
TENEFVVRFSHRRYPARPDQPPLRQGSFGVGGAGGGEEFGLGAEERVEVLEDADDAGSVVAVLTVSLVAEAALATLRETMRHRGGGGGVPY

SEQ ID NO: 157 - DNA, 345 bp, Chlamydomonas reinhardtii
atgccgctgatcgtggacgcgctgctgccgccgggcgtggccagggaggcctttccgttccgccggcccaactactggggccagctggtgcagctggtacgggccggcatcagcgcagggcagctgcagctcgaccacgtttgggatatcagcgcagcagaccaggagcaggtgctcagcacctgcctgggccccatatcttccgggatctgcgccaagatggctgccgactacgccattggggcgcacgtgaacatcaaggacctcatggagcaggcggcggcgttagagcaggcggcggcggtgcagcaggcggcagctggagcaggcggtgacatgcagtag

SEQ ID NO: 158 - protein, 114 aa, Chlamydomonas reinhardtii
MPLIVDALLPPGVAREAFPFRRPNYWGQLVQLVRAGISAGQLQLDHVWDISAADQEQVLSTCLGPISSGICAKMAADYAIGAHVNIKDLMEQAAALEQAAAVQQAAAGAGGDMQ

SEQ ID NO: 159 - DNA, 354 bp, artificial sequence
  codon optimized
ctcgagcctctcattgtggatgcactgctgccgcctggtgtcgctcgggaggcgttcccctttcgccggccgaactactggggccaactggtccagctggtccgggccgggatcagcgccggccagctccagctcgatcacgtgtgggacatctcggctgcggaccaggagcaagtgctctcgacctgcctcggcccgatcagcagcggcatttgcgcgaagatggcggcggactacgccatcggcgcgcacgtgaacatcaaggacctgatggagcaggctgctgccctggagcaggccgcagccgtgcagcaggcggctgccggtgccggcggcgacatgcaatagggatcc

SEQ ID NO: 160 - DNA, 339 bp, artificial sequence
  codon optimized
cctctcattgtggatgcactgctgccgcctggtgtcgctcgggaggcgttcccctttcgccggccgaactactggggccaactggtccagctggtccgggccgggatcagcgccggccagctccagctcgatcacgtgtgggacatctcggctgcggaccaggagcaagtgctctcgacctgcctcggcccgatcagcagcggcatttgcgcgaagatggcggcggactacgccatcggcgcgcacgtgaacatcaaggacctgatggagcaggctgctgccctggagcaggccgcagccgtgcagcaggcggctgccggtgccggcggcgacatgcaa

SEQ ID NO: 161 - DNA, 339 bp, Chlamydomonas reinhardtii
ccgctgatcgtggacgcgctgctgccgccgggcgtggccagggaggcctttccgttccgccggcccaactactggggccagctggtgcagctggtacgggccggcatcagcgcagggcagctgcagctcgaccacgtttgggatatcagcgcagcagaccaggagcaggtgctcagcacctgcctgggccccatatcttccgggatctgcgccaagatggctgccgactacgccattggggcgcacgtgaacatcaaggacctcatggagcaggcggcggcgttagagcaggcggcggcggtgcagcaggcggcagctggagcaggcggtgacatgcag

SEQ ID NO: 162 - protein, 113 aa, Chlamydomonas reinhardtii
PLIVDALLPPGVAREAFPFRRPNYWGQLVQLVRAGISAGQLQLDHVWDISAADQEQVLSTCLGPISSGICAKMAADYAIGAHVNIKDLMEQAAALEQAAAVQQAAAGAGGDMQ

SEQ ID NO: 163 - DNA, 156 bp, Chlamydomonas reinhardtii
atggccggcgtaggggctgtcgtgaagctccctgataatgcgggtgcgcaaatcgttgtagttagggacaacgattttgccttggcagcggtagaaaccatcagtttggtcgtaggtgacaccgtggcgggcgagtgcccttgcgtcgacgaatag

SEQ ID NO: 164 - protein, 51 aa, Chlamydomonas reinhardtii
MAGVGAVVKLPDNAGAQIVVVRDNDFALAAVETISLVVGDTVAGECPCVDE

SEQ ID NO: 165 - DNA, 165 bp, artificial sequence
  codon optimized
ctcgaggcaggtgtgggtgcagtggtcaagctccccgataacgcaggcgcacagatcgtggtggtgcgcgacaacgacttcgcgctggcggccgtggagacgatctccctggtggtgggcgacaccgtggctggcgagtgtccctgcgtggacgagtaaggatcc

SEQ ID NO: 166 - DNA, 150 bp, artificial sequence
  codon optimized
gcaggtgtgggtgcagtggtcaagctccccgataacgcaggcgcacagatcgtggtggtgcgcgacaacgacttcgcgctggcggccgtggagacgatctccctggtggtgggcgacaccgtggctggcgagtgtccctgcgtggacgag

SEQ ID NO: 167 - DNA, 150 bp, Chlamydomonas reinhardtii
gccggcgtaggggctgtcgtgaagctccctgataatgcgggtgcgcaaatcgttgtagttagggacaacgattttgccttggcagcggtagaaaccatcagtttggtcgtaggtgacaccgtggcgggcgagtgcccttgcgtcgacgaa

SEQ ID NO: 168 - protein, 50 aa, Chlamydomonas reinhardtii
AGVGAVVKLPDNAGAQIVVVRDNDFALAAVETISLVVGDTVAGECPCVDE

SEQ ID NO: 169 - DNA, 291 bp, Chlamydomonas reinhardtii
atgtcattctccggctccgctctgttcgcaaccatgcacgatcgtatcaccacaagcgagcgagccgctcaacctacacacagtatctacggattaggtcatgccagcaagacagccaagacacagtacagtcgcagcgccgagcaggcgcgcggtaccaatacgcaggctgcagtgtgcgggtgcgggtgcaccggggctctggcgcctggaccccctgctgaagcatgcatgtatcctcaaatttgtcgcgctacttacaacaaacaccgcacactaaccaccctatag

SEQ ID NO: 170 - protein, 96 aa, Chlamydomonas reinhardtii
MSFSGSALFATMHDRITTSERAAQPTHSIYGLGHASKTAKTQYSRSAEQARGTNTQAAVCGCGCTGALAPGPPAEACMYPQICRATYNKHRTLTTL

SEQ ID NO: 171 - DNA, 297 bp, artificial sequence
  codon optimized
ctcgagagcttctcggggagcgccctgttcgcgactatgcacgatcgcatcaccacctccgagcgcgccgcccagccaacccactccatctacggcctcggccacgcctccaagactgcgaagacccagtatagccgctccgccgagcaggcgcgtggcaccaacacccaggccgccgtgtgcggctgcggctgcaccggcgcgctggcacccggccctcctgcggaggcgtgcatgtacccccagatttgccgcgccacctacaacaagcaccgtaccctgaccacgctgaccggt

SEQ ID NO: 172 - DNA, 285 bp, artificial sequence
  codon optimized
agcttctcggggagcgccctgttcgcgactatgcacgatcgcatcaccacctccgagcgcgccgcccagccaacccactccatctacggcctcggccacgcctccaagactgcgaagacccagtatagccgctccgccgagcaggcgcgtggcaccaacacccaggccgccgtgtgcggctgcggctgcaccggcgcgctggcacccggccctcctgcggaggcgtgcatgtacccccagatttgccgcgccacctacaacaagcaccgtaccctgaccacgctg

SEQ ID NO: 173 - DNA, 285 bp, Chlamydomonas reinhardtii
tcattctccggctccgctctgttcgcaaccatgcacgatcgtatcaccacaagcgagcgagccgctcaacctacacacagtatctacggattaggtcatgccagcaagacagccaagacacagtacagtcgcagcgccgagcaggcgcgcggtaccaatacgcaggctgcagtgtgcgggtgcgggtgcaccggggctctggcgcctggaccccctgctgaagcatgcatgtatcctcaaatttgtcgcgctacttacaacaaacaccgcacactaaccacccta

SEQ ID NO: 174 - protein, 95 aa, Chlamydomonas reinhardtii
SFSGSALFATMHDRITTSERAAQPTHSIYGLGHASKTAKTQYSRSAEQARGTNTQAAVCGCGCTGALAPGPPAEACMYPQICRATYNKHRTLTTL

SEQ ID NO: 175 - DNA, 537 bp, Chlamydomonas reinhardtii
atggcagacgtagaggacgattcgggcggcggagtggacgcgggcggaggcgacgcagaggttgcggtggcacgtcaggcggagcctgcggccaagcggcggacctcggagaccgcggagggcgccaagcccgctcacaagctcccggcgcaggctgacgaggtcaggcgcaaggaggaggagcaggcgaggctggaggcgcggcggctggccatccgggagcgcattgccaagcagcacgaggtggctgccaagctggctgaggagcgcgccaaggctgaggaggccgccaagggtgccatcgaccgattcctggccgcaaagcccctgcacctcaaactgctggaggagtacgagaagcagcaggcggagctggaggcggagaagaagcgcgcctacgaggagggggaggcgcggaacaagctggtgcggccgcaccagatcatgtcagggcagacccgaggcccctgccactacggtaccgctcacgcatcaccaccactgtacgaactcgcgccgggccaggctcgcccgtga

SEQ ID NO: 176 - protein, 178 aa, Chlamydomonas reinhardtii
MADVEDDSGGGVDAGGGDAEVAVARQAEPAAKRRTSETAEGAKPAHKLPAQADEVRRKEEEQARLEARRLAIRERIAKQHEVAAKLAEERAKAEEAAKGAIDRFLAAKPLHLKLLEEYEKQQAELEAEKKRAYEEGEARNKLVRPHQIMSGQTRGPCHYGTAHASPPLYELAPGQARP

SEQ ID NO: 177 - DNA, 543 bp, artificial sequence
  codon optimized
catatggctgatgtggaggatgattccggtggtggtgtcgatgctggtggtggtgacgccgaggtggccgtggcccggcaagccgagcccgcagcaaagcgccgcacgagcgagacagcagagggcgctaagcccgcgcataagctcccagcacaggcagacgaggtgcgccgtaaggaggaggagcaggcgcgtctggaggctcggcgcctggcgattcgcgagcgtatcgcaaagcagcacgaggtcgccgcgaagctggcagaggagcgggcgaaggcggaggaggccgcgaagggtgctatcgaccgtttcctggctgctaagccgctccacctcaagctgctggaggagtacgagaagcagcaggcggagctggaggcggagaagaagcgcgcctacgaggagggcgaggcccgcaacaagctcgtgcgcccgcaccagatcatgtcgggccagacacgtggcccctgccactacggcactgcccacgcgagcccgccgctgtacgagctcgcccctggccaagcccgcccaaccggt

SEQ ID NO: 178 - DNA, 531 bp, artificial sequence
  codon optimized
gctgatgtggaggatgattccggtggtggtgtcgatgctggtggtggtgacgccgaggtggccgtggcccggcaagccgagcccgcagcaaagcgccgcacgagcgagacagcagagggcgctaagcccgcgcataagctcccagcacaggcagacgaggtgcgccgtaaggaggaggagcaggcgcgtctggaggctcggcgcctggcgattcgcgagcgtatcgcaaagcagcacgaggtcgccgcgaagctggcagaggagcgggcgaaggcggaggaggccgcgaagggtgctatcgaccgtttcctggctgctaagccgctccacctcaagctgctggaggagtacgagaagcagcaggcggagctggaggcggagaagaagcgcgcctacgaggagggcgaggcccgcaacaagctcgtgcgcccgcaccagatcatgtcgggccagacacgtggcccctgccactacggcactgcccacgcgagcccgccgctgtacgagctcgcccctggccaagcccgccca

SEQ ID NO: 179 - DNA, 531 bp, Chlamydomonas reinhardtii
gcagacgtagaggacgattcgggcggcggagtggacgcgggcggaggcgacgcagaggttgcggtggcacgtcaggcggagcctgcggccaagcggcggacctcggagaccgcggagggcgccaagcccgctcacaagctcccggcgcaggctgacgaggtcaggcgcaaggaggaggagcaggcgaggctggaggcgcggcggctggccatccgggagcgcattgccaagcagcacgaggtggctgccaagctggctgaggagcgcgccaaggctgaggaggccgccaagggtgccatcgaccgattcctggccgcaaagcccctgcacctcaaactgctggaggagtacgagaagcagcaggcggagctggaggcggagaagaagcgcgcctacgaggagggggaggcgcggaacaagctggtgcggccgcaccagatcatgtcagggcagacccgaggcccctgccactacggtaccgctcacgcatcaccaccactgtacgaactcgcgccgggccaggctcgcccg

SEQ ID NO: 180 - protein, 177 aa, Chlamydomonas reinhardtii
ADVEDDSGGGVDAGGGDAEVAVARQAEPAAKRRTSETAEGAKPAHKLPAQADEVRRKEEEQARLEARRLAIRERIAKQHEVAAKLAEERAKAEEAAKGAIDRFLAAKPLHLKLLEEYEKQQAELEAEKKRAYEEGEARNKLVRPHQIMSGQTRGPCHYGTAHASPPLYELAPGQARP

SEQ ID NO: 181 - DNA, 528 bp, Chlamydomonas reinhardtii
atgtcatcccttttatcagctggcactggctcgacttccacgcccgtgagcctagcaaaactcagcgcagcacgattggccaaccgtttcgtggagctgggcaagctacgtgacttggccccccgcgagtgcattctgctcacatgccgcggtctgtgcctgtcggcggacaccactggccgcctggcgctgcacatatggactcgggtagaaccgctggcgcgcctgttgctgtctgtgcggccagacgccaccgccgccgctgccccgctggctgtctacggctgtcggggcgctggtgctgcggccgcggggctgcaggtgtatgccgtggcggctgtgtgggtggcagcgaagcttgaggagcggcgtcaggaggtgccgggcagtggcgcgctagcggtggccgcgcgcagcagccccgcggccctggcggcagctgagctgcgcattctgcagtggtgcgactgggccccgtacactgggtttgtgcctgacgagtcccacctgctggtgtgggcaccctga

SEQ ID NO: 182 - protein, 175 aa, Chlamydomonas reinhardtii
MSSLLSAGTGSTSTPVSLAKLSAARLANRFVELGKLRDLAPRECILLTCRGLCLSADTTGRLALHIWTRVEPLARLLLSVRPDATAAAAPLAVYGCRGAGAAAAGLQVYAVAAVWVAAKLEERRQEVPGSGALAVAARSSPAALAAAELRILQWCDWAPYTGFVPDESHLLVWAP

SEQ ID NO: 183 - DNA, 549 bp, artificial sequence
  codon optimized
catatgctcgagtcgagcctgctgagcgctggtactggttccacttccactcctgtgtccctggccaagctgtcggctgcgcggctcgctaaccgcttcgtggagctgggcaagctgcgcgatctggctccgcgggagtgtatcctgctcacctgtcgcgggctctgcctgagcgcggacaccacgggccgcctggccctgcacatctggacccgcgtcgagcccctggcgcgcctgctcctgtcggtgcgccccgacgcgacagcggcagctgctccgctggcggtgtacggctgtcgcggcgcaggcgctgccgcggcggggctgcaggtctacgcggtggcggcagtgtgggtggcggcgaagctggaggagcgccgtcaggaggtgcctggcagcggcgccctggcggtggcggcccggtcctccccggcggctctggccgcggccgagctgcgcatcctgcaatggtgcgactgggccccgtacacggggtttgtcccggacgagtcccacctcctggtctgggcgccgaccggttaaggatcc

SEQ ID NO: 184 - DNA, 522 bp, artificial sequence
  codon optimized
tcgagcctgctgagcgctggtactggttccacttccactcctgtgtccctggccaagctgtcggctgcgcggctcgctaaccgcttcgtggagctgggcaagctgcgcgatctggctccgcgggagtgtatcctgctcacctgtcgcgggctctgcctgagcgcggacaccacgggccgcctggccctgcacatctggacccgcgtcgagcccctggcgcgcctgctcctgtcggtgcgccccgacgcgacagcggcagctgctccgctggcggtgtacggctgtcgcggcgcaggcgctgccgcggcggggctgcaggtctacgcggtggcggcagtgtgggtggcggcgaagctggaggagcgccgtcaggaggtgcctggcagcggcgccctggcggtggcggcccggtcctccccggcggctctggccgcggccgagctgcgcatcctgcaatggtgcgactgggccccgtacacggggtttgtcccggacgagtcccacctcctggtctgggcgccg

SEQ ID NO: 185 - DNA, 522 bp, Chlamydomonas reinhardtii
tcatcccttttatcagctggcactggctcgacttccacgcccgtgagcctagcaaaactcagcgcagcacgattggccaaccgtttcgtggagctgggcaagctacgtgacttggccccccgcgagtgcattctgctcacatgccgcggtctgtgcctgtcggcggacaccactggccgcctggcgctgcacatatggactcgggtagaaccgctggcgcgcctgttgctgtctgtgcggccagacgccaccgccgccgctgccccgctggctgtctacggctgtcggggcgctggtgctgcggccgcggggctgcaggtgtatgccgtggcggctgtgtgggtggcagcgaagcttgaggagcggcgtcaggaggtgccgggcagtggcgcgctagcggtggccgcgcgcagcagccccgcggccctggcggcagctgagctgcgcattctgcagtggtgcgactgggccccgtacactgggtttgtgcctgacgagtcccacctgctggtgtgggcaccc

SEQ ID NO: 186 - protein, 174 aa, Chlamydomonas reinhardtii
SSLLSAGTGSTSTPVSLAKLSAARLANRFVELGKLRDLAPRECILLTCRGLCLSADTTGRLALHIWTRVEPLARLLLSVRPDATAAAAPLAVYGCRGAGAAAAGLQVYAVAAVWVAAKLEERRQEVPGSGALAVAARSSPAALAAAELRILQWCDWAPYTGFVPDESHLLVWAP

SEQ ID NO: 187 - DNA, 525 bp, Chlamydomonas reinhardtii
atgcccgtccaggtcctcgagcaaacggcgcagcagctggctgggggaaggcccaaggaggaggtcatcgtcggctggggcaacgccaacactggccacggcggctgcgtcagcaggtcaggcaggggcccaaaccgggcgctgctgcgtctgctggtggacaagtacgcccacctggtggtctacctggacgaatactacaccagccagccctgcgacctgctggcggcggtggcggtgcagggcctggcggcggtggtggcagcggtgcagggcctggagacggcggtggcggcggcggtggcaccgcgcctaccggcggcaacagcagcggccacggcgttgggcccggtggtggcggcggcacgggcggccctgggcccagcggcagtggcggcgcgcaagtttggagcagagggggcgggcaaggccacgtggaggaggacagcgcggcgccgccttcaaagcggcgccggcgcgcaggttgagacctgccgttgttgtctggttagcgacctgtgctga

SEQ ID NO: 188 - protein, 174 aa, Chlamydomonas reinhardtii
MPVQVLEQTAQQLAGGRPKEEVIVGWGNANTGHGGCVSRSGRGPNRALLRLLVDKYAHLVVYLDEYYTSQPCDLLAAVAVQGLAAVVAAVQGLETAVAAAVAPRLPAATAAATALGPVVAAARAALGPAAVAARKFGAEGAGKATWRRTARRRLQSGAGAQVETCRCCLVSDLC

SEQ ID NO: 189 - DNA, 540 bp, artificial sequence
  codon optimized
catatgctcgagcctgtgcaggtcctggagcaaacggctcaacaactggcaggcggtcggcctaaggaggaggtgattgtgggctggggcaacgctaataccggccacggcggctgcgtgtcccgtagcggtcggggcccaaaccgcgcgctgctgcggctgctcgtggacaagtatgcccacctggtggtctacctggacgagtactacacctcccagccgtgcgacctgctcgcggcggtggccgtgcaggggctggcggctgtggtggccgcggtgcagggcctggagacggccgtggcggctgccgtcgccccacgcctgccagccgcgactgcggcggcgacagccctgggcccggtggtggctgctgcccgcgctgcactggggcctgccgcggtcgcagcgcgcaagttcggcgcggagggtgccggcaaggctacatggcggcgtaccgcgcgccgccgcctgcaaagcggcgcaggcgcgcaggtggagacttgccggtgctgcctggtgtcggacctgtgctagggatcc

SEQ ID NO: 190 - DNA, 519 bp, artificial sequence
  codon optimized
cctgtgcaggtcctggagcaaacggctcaacaactggcaggcggtcggcctaaggaggaggtgattgtgggctggggcaacgctaataccggccacggcggctgcgtgtcccgtagcggtcggggcccaaaccgcgcgctgctgcggctgctcgtggacaagtatgcccacctggtggtctacctggacgagtactacacctcccagccgtgcgacctgctcgcggcggtggccgtgcaggggctggcggctgtggtggccgcggtgcagggcctggagacggccgtggcggctgccgtcgccccacgcctgccagccgcgactgcggcggcgacagccctgggcccggtggtggctgctgcccgcgctgcactggggcctgccgcggtcgcagcgcgcaagttcggcgcggagggtgccggcaaggctacatggcggcgtaccgcgcgccgccgcctgcaaagcggcgcaggcgcgcaggtggagacttgccggtgctgcctggtgtcggacctgtgc

SEQ ID NO: 191 - DNA, 519 bp, Chlamydomonas reinhardtii
cccgtccaggtcctcgagcaaacggcgcagcagctggctgggggaaggcccaaggaggaggtcatcgtcggctggggcaacgccaacactggccacggcggctgcgtcagcaggtcaggcaggggcccaaaccgggcgctgctgcgtctgctggtggacaagtacgcccacctggtggtctacctggacgaatactacaccagccagccctgcgacctgctggcggcggtggcggtgcagggcctggcggcggtggtggcagcggtgcagggcctggagacggcggtggcggcggcggtggcaccgcgcctaccggcggcaacagcagcggccacggcgttgggcccggtggtggcggcggcacgggcggccctgggcccagcggcagtggcggcgcgcaagtttggagcagagggggcgggcaaggccacgtggaggaggacagcgcggcgccgccttcaaagcggcgccggcgcgcaggttgagacctgccgttgttgtctggttagcgacctgtgc

SEQ ID NO: 192 - protein, 173 aa, Chlamydomonas reinhardtii
PVQVLEQTAQQLAGGRPKEEVIVGWGNANTGHGGCVSRSGRGPNRALLRLLVDKYAHLVVYLDEYYTSQPCDLLAAVAVQGLAAVVAAVQGLETAVAAAVAPRLPAATAAATALGPVVAAARAALGPAAVAARKFGAEGAGKATWRRTARRRLQSGAGAQVETCRCCLVSDLC

SEQ ID NO: 193 - DNA, 279 bp, Chlamydomonas reinhardtii
atgaagatggcaagcgaggtatggaccaagggtggctatgctcacgtgcttggcagcggtggcagtaggttcacaggcagcgacgacgcggtcctgtgcggctaccgccgtgaggatgtgcctcagccctgggaggccgtgccggcgggcttcatgctgggtggactggtcgcggagcgtgccgccaacgcctccacattcagccgcgtcggctttgtgtttgtggccgagccgtccgcagaggagtgggcctcaatggcttcaatcaaaggcgagtag

SEQ ID NO: 194 - protein, 92 aa, Chlamydomonas reinhardtii
MKMASEVWTKGGYAHVLGSGGSRFTGSDDAVLCGYRREDVPQPWEAVPAGFMLGGLVAERAANASTFSRVGFVFVAEPSAEEWASMASIKGE

SEQ ID NO: 195 - DNA, 294 bp, artificial sequence
  codon optimized
catatgctcgagaagatggcttccgaggtgtggactaagggtggttatgctcatgtgctgggttccggcggcagccgctttacggggtcggatgacgcggtcctctgcggctaccgccgtgaggacgtgccgcagccctgggaggcggtgcccgcaggcttcatgctgggcggcctggtggccgagcgtgccgccaacgcgagcacgtttagccgcgtgggcttcgtgttcgtggcagagcccagcgccgaggagtgggcgtcgatggcgagcatcaagggtgagtaaggatcc

SEQ ID NO: 196 - DNA, 273 bp, artificial sequence
  codon optimized
aagatggcttccgaggtgtggactaagggtggttatgctcatgtgctgggttccggcggcagccgctttacggggtcggatgacgcggtcctctgcggctaccgccgtgaggacgtgccgcagccctgggaggcggtgcccgcaggcttcatgctgggcggcctggtggccgagcgtgccgccaacgcgagcacgtttagccgcgtgggcttcgtgttcgtggcagagcccagcgccgaggagtgggcgtcgatggcgagcatcaagggtgag

SEQ ID NO: 197 - DNA, 273 bp, Chlamydomonas reinhardtii
aagatggcaagcgaggtatggaccaagggtggctatgctcacgtgcttggcagcggtggcagtaggttcacaggcagcgacgacgcggtcctgtgcggctaccgccgtgaggatgtgcctcagccctgggaggccgtgccggcgggcttcatgctgggtggactggtcgcggagcgtgccgccaacgcctccacattcagccgcgtcggctttgtgtttgtggccgagccgtccgcagaggagtgggcctcaatggcttcaatcaaaggcgag

SEQ ID NO: 198 - protein, 91 aa, Chlamydomonas reinhardtii
KMASEVWTKGGYAHVLGSGGSRFTGSDDAVLCGYRREDVPQPWEAVPAGFMLGGLVAERAANASTFSRVGFVFVAEPSAEEWASMASIKGE

SEQ ID NO: 199 - DNA, 972 bp, Chlamydomonas reinhardtii
atgccggcgctgaaagccgacgtcctagcacccaccaacggatccagcctagccgctgacggtggcggcagccgccagcaggagcggcgccggtcggtcacgggcgcagtggtttgcaactgctacggtgagcctgcccgggacgaggcactggaacagctgctggctcgccgtcgcggtgctgctgggggcggtggcggacacggagacggcggcggaggcgtggacgcaggcggcggctttgtggggctgtgggcggactacgctatgttgaaccacagctgctgccctaacacgatcaactgggtgggcgggccgcacgaccacatggcagtcatcgccacggcgcccatcgccgcggggcaggagcgcagccacaacttcacttgcgcctgccgccgctgcgagcacgagcggcgcctgggctcggccctggaggccgccctgcagcatgtgtacgacagcgtgaacgtggagtgggggccgcggctgggcgcgctggcagaggaggtccaggatgcggcggaggcgctgcgggaattgcggcggcaacaggcgcaggcgcaggcgctgacaggtcaaggcaggcaggggcaggcgtcgcgcggcctcttcggtgcactgtttggcgctgtgggaggcggtggtgtggtcgcggcggcggcaacgatgcgggagcctgtggaacgcgcgcgggcggcgctgtcttcactgtcaaacgagcttgcagcgctcgaccagcaggttgcggcgaccctatccgacgttgaaatggcagcatcgcaagctgccgccgggaatgccgccgctgccaccaccgcagccgaggccgggtggtgggtgcgggccagcctttacgacgcctatgagctgcgggcatccattgcagaggtgctggccgacaccgcacgcgcagctgcgtcgacaacagcatcggcgccaggggccacactcagccccaaggtcacctag

SEQ ID NO: 200 - protein, 323 aa, Chlamydomonas reinhardtii
MPALKADVLAPTNGSSLAADGGGSRQQERRRSVTGAVVCNCYGEPARDEALEQLLARRRGAAGGGGGHGDGGGGVDAGGGFVGLWADYAMLNHSCCPNTINWVGGPHDHMAVIATAPIAAGQERSHNFTCACRRCEHERRLGSALEAALQHVYDSVNVEWGPRLGALAEEVQDAAEALRELRRQQAQAQALTGQGRQGQASRGLFGALFGAVGGGGVVAAAATMREPVERARAALSSLSNELAALDQQVAATLSDVEMAASQAAAGNAAAATTAAEAGWWVRASLYDAYELRASIAEVLADTARAAASTTASAPGATLSPKVT

SEQ ID NO: 201 - DNA, 993 bp, artificial sequence
  codon optimized
catatgctcgagccagctctgaaggccgatgtcctcgctccaacaaatggtagcagcctcgccgcagacggcggtggcagccgccaacaggagcgtcgccgtagcgtgacaggcgccgtcgtctgcaactgctacggggagccggcgcgcgacgaggcgctggagcaactgctggctcgtcggcggggtgcggctggcggcggtggtggccacggcgacggtggtggcggcgtggacgctggcggcggcttcgtcggcctgtgggcggactacgccatgctgaaccactcctgctgccccaacaccatcaactgggtggggggcccccacgaccacatggctgtgatcgcgaccgcgcccatcgccgctggccaggagcgctcgcacaatttcacctgcgcttgccgccgctgcgagcacgagcgtcggctcggcagcgccctggaggccgccctccagcacgtgtacgacagcgtcaacgtggagtgggggccacggctgggcgcgctcgctgaggaggtgcaggacgctgcggaggccctccgcgagctgcgccggcagcaggcgcaggcccaggccctgacaggccagggccggcagggtcaggcctcgcgtggcctctttggcgcgctgtttggcgcggtgggcggtggtggcgtggtggctgccgctgctacaatgcgcgagcccgtcgagcgcgcacgggctgcgctgtcctcgctgtcgaacgagctggccgcgctggaccagcaggtcgccgcgaccctgagcgacgtggagatggcggccagccaggcagcggctggcaacgcagccgctgcgacgaccgccgccgaggcgggctggtgggtgcgtgcatccctgtacgacgcttatgagctgcgcgcgtccattgcagaggtgctggcggacacggcccgtgccgccgcatcgacaacggccagcgcccccggcgcaaccctgagcccgaaggtcaccaccggttaaggatcc

SEQ ID NO: 202 - DNA, 966 bp, artificial sequence
  codon optimized
ccagctctgaaggccgatgtcctcgctccaacaaatggtagcagcctcgccgcagacggcggtggcagccgccaacaggagcgtcgccgtagcgtgacaggcgccgtcgtctgcaactgctacggggagccggcgcgcgacgaggcgctggagcaactgctggctcgtcggcggggtgcggctggcggcggtggtggccacggcgacggtggtggcggcgtggacgctggcggcggcttcgtcggcctgtgggcggactacgccatgctgaaccactcctgctgccccaacaccatcaactgggtggggggcccccacgaccacatggctgtgatcgcgaccgcgcccatcgccgctggccaggagcgctcgcacaatttcacctgcgcttgccgccgctgcgagcacgagcgtcggctcggcagcgccctggaggccgccctccagcacgtgtacgacagcgtcaacgtggagtgggggccacggctgggcgcgctcgctgaggaggtgcaggacgctgcggaggccctccgcgagctgcgccggcagcaggcgcaggcccaggccctgacaggccagggccggcagggtcaggcctcgcgtggcctctttggcgcgctgtttggcgcggtgggcggtggtggcgtggtggctgccgctgctacaatgcgcgagcccgtcgagcgcgcacgggctgcgctgtcctcgctgtcgaacgagctggccgcgctggaccagcaggtcgccgcgaccctgagcgacgtggagatggcggccagccaggcagcggctggcaacgcagccgctgcgacgaccgccgccgaggcgggctggtgggtgcgtgcatccctgtacgacgcttatgagctgcgcgcgtccattgcagaggtgctggcggacacggcccgtgccgccgcatcgacaacggccagcgcccccggcgcaaccctgagcccgaaggtcacc

SEQ ID NO: 203 - DNA, 966 bp, Chlamydomonas reinhardtii
ccggcgctgaaagccgacgtcctagcacccaccaacggatccagcctagccgctgacggtggcggcagccgccagcaggagcggcgccggtcggtcacgggcgcagtggtttgcaactgctacggtgagcctgcccgggacgaggcactggaacagctgctggctcgccgtcgcggtgctgctgggggcggtggcggacacggagacggcggcggaggcgtggacgcaggcggcggctttgtggggctgtgggcggactacgctatgttgaaccacagctgctgccctaacacgatcaactgggtgggcgggccgcacgaccacatggcagtcatcgccacggcgcccatcgccgcggggcaggagcgcagccacaacttcacttgcgcctgccgccgctgcgagcacgagcggcgcctgggctcggccctggaggccgccctgcagcatgtgtacgacagcgtgaacgtggagtgggggccgcggctgggcgcgctggcagaggaggtccaggatgcggcggaggcgctgcgggaattgcggcggcaacaggcgcaggcgcaggcgctgacaggtcaaggcaggcaggggcaggcgtcgcgcggcctcttcggtgcactgtttggcgctgtgggaggcggtggtgtggtcgcggcggcggcaacgatgcgggagcctgtggaacgcgcgcgggcggcgctgtcttcactgtcaaacgagcttgcagcgctcgaccagcaggttgcggcgaccctatccgacgttgaaatggcagcatcgcaagctgccgccgggaatgccgccgctgccaccaccgcagccgaggccgggtggtgggtgcgggccagcctttacgacgcctatgagctgcgggcatccattgcagaggtgctggccgacaccgcacgcgcagctgcgtcgacaacagcatcggcgccaggggccacactcagccccaaggtcacc

SEQ ID NO: 204 - protein, 322 aa, Chlamydomonas reinhardtii
PALKADVLAPTNGSSLAADGGGSRQQERRRSVTGAVVCNCYGEPARDEALEQLLARRRGAAGGGGGHGDGGGGVDAGGGFVGLWADYAMLNHSCCPNTINWVGGPHDHMAVIATAPIAAGQERSHNFTCACRRCEHERRLGSALEAALQHVYDSVNVEWGPRLGALAEEVQDAAEALRELRRQQAQAQALTGQGRQGQASRGLFGALFGAVGGGGVVAAAATMREPVERARAALSSLSNELAALDQQVAATLSDVEMAASQAAAGNAAAATTAAEAGWWVRASLYDAYELRASIAEVLADTARAAASTTASAPGATLSPKVT

SEQ ID NO: 205 - DNA, 264 bp, Chlamydomonas reinhardtii
atgcacctcaccctggtcattgtggcggcggtggcggtcaccgtcatcctcattgtcaccttcgtgcagggcacgctgacgacgttcacgcccgagaacgccgacctccaagtgtttgtggtgtgggtcatcccagcgttcatctacctggtggcgggtgtggtgctggacgtcatcatgtcggcctccacgggccagatgtaccacgacgacgtccaggtgctcacctcggaggcgacatttacgcacaattacacgccgtga

SEQ ID NO: 206 - protein, 87 aa, Chlamydomonas reinhardtii
MHLTLVIVAAVAVTVILIVTFVQGTLTTFTPENADLQVFVVWVIPAFIYLVAGVVLDVIMSASTGQMYHDDVQVLTSEATFTHNYTP

SEQ ID NO: 207 - DNA, 285 bp, artificial sequence
  codon optimized
catatgctcgagcatctgactctcgtgattgtcgctgctgtcgctgtgactgtgattctgattgtcacattcgtccagggcacactgaccacgttcaccccggagaacgccgatctccaggtgttcgtggtgtgggtgattccggcctttatttacctggtcgccggtgtcgtgctggacgtgatcatgagcgcctccaccggccagatgtaccacgacgacgtgcaggtgctgacctcggaggccaccttcacccacaactacacgccgaccggttgaggatcc

SEQ ID NO: 208 - DNA, 258 bp, artificial sequence
  codon optimized
catctgactctcgtgattgtcgctgctgtcgctgtgactgtgattctgattgtcacattcgtccagggcacactgaccacgttcaccccggagaacgccgatctccaggtgttcgtggtgtgggtgattccggcctttatttacctggtcgccggtgtcgtgctggacgtgatcatgagcgcctccaccggccagatgtaccacgacgacgtgcaggtgctgacctcggaggccaccttcacccacaactacacgccg

SEQ ID NO: 209 - DNA, 258 bp, Chlamydomonas reinhardtii
cacctcaccctggtcattgtggcggcggtggcggtcaccgtcatcctcattgtcaccttcgtgcagggcacgctgacgacgttcacgcccgagaacgccgacctccaagtgtttgtggtgtgggtcatcccagcgttcatctacctggtggcgggtgtggtgctggacgtcatcatgtcggcctccacgggccagatgtaccacgacgacgtccaggtgctcacctcggaggcgacatttacgcacaattacacgccg

SEQ ID NO: 210 - protein, 86 aa, Chlamydomonas reinhardtii
HLTLVIVAAVAVTVILIVTFVQGTLTTFTPENADLQVFVVWVIPAFIYLVAGVVLDVIMSASTGQMYHDDVQVLTSEATFTHNYTP

SEQ ID NO: 211 - DNA, 729 bp, Chlamydomonas reinhardtii
atgaccggcaagcacgacgcggcaagcacggctgtagtgttccctggagccgcggaggagtcgccgccgcgccccacccaacagcacgagcagcggctgtgctgccgcatcggccccaccggctcgcacggcgcgctggccctgcaggtgcctctggcgggccgcatggagggcgccgtggaggcggtgcgggacgtgttggcggaggcgctgggcaggctgtggcggcagccggaggcggcgggtgcatgcgcaggcggccaacagccgcaggcgcagccgcagccggcacctcgccacgaccaccgccagcaggagcaacagagggatctgccacggcgccaagatcacctgcggtcgccgccgcagcagctgctgctgctgcccgaccagcagcgtcccaatgagggtaaagaggagggggagggtggagccgtcgtgcaggcacgggtgcagcatgtatcccgtgctgctgaggccggagccgcaggccccgcacgtgagggcgtgagggtgaatgcgcagccgccagctgcggagcccgccccggccgccgccgctgccaagggcagccgcagccgcagcggcaacaggggcagatatggcaacagcagcagcaacggcatcagcagcagcggcggcggcagcagcggcggtagcagttgtgagagcatgcgggcgccgtcatggaacagccacgtggaagcggctggcaagccggctacatga

SEQ ID NO: 212 - protein, 242 aa, Chlamydomonas reinhardtii
MTGKHDAASTAVVFPGAAEESPPRPTQQHEQRLCCRIGPTGSHGALALQVPLAGRMEGAVEAVRDVLAEALGRLWRQPEAAGACAGGQQPQAQPQPAPRHDHRQQEQQRDLPRRQDHLRSPPQQLLLLPDQQRPNEGKEEGEGGAVVQARVQHVSRAAEAGAAGPAREGVRVNAQPPAAEPAPAAAAAKGSRSRSGNRGRYGNSSSNGISSSGGGSSGGSSCESMRAPSWNSHVEAAGKPAT

SEQ ID NO: 213 - DNA, 744 bp, artificial sequence
  codon optimized
catatgctcgagactggtaagcatgacgctgcaagcactgcggtggtgtttcctggcgctgctgaggagagcccgcctcgccctacgcagcagcacgagcagcgcctctgctgccgcattggccctacggggtcgcacggtgccctggccctgcaagtgcccctcgccggccgcatggagggcgccgtggaggcagtccgcgacgtgctggcggaggctctgggccgcctgtggcgccagcccgaggcagccggcgcttgcgccggcggccagcagccccaggcccaaccgcagcctgcgccccgccacgaccaccggcaacaggagcagcagcgcgacctcccccgccgtcaggatcatctgcggagcccgccacagcagctcctgctcctgcccgaccagcagcgcccgaacgagggcaaggaggagggggagggtggcgccgtggtgcaggctcgggtgcagcacgtcagccgggctgccgaggcgggtgcggctggccccgcccgcgagggcgtgcgtgtcaacgcgcagcctcctgccgcagagccggcccctgctgccgctgccgcgaagggcagccgctcccgctccggcaaccggggtcgttacggtaacagctcgtcgaacggcatctcgtcctccggcggtggctcgtccggcggttcgtcctgcgagtcgatgcgggcccccagctggaactcccacgtcgaggccgcaggcaagcccgcaacctgaggatcc

SEQ ID NO: 214 - DNA, 723 bp, artificial sequence
  codon optimized
actggtaagcatgacgctgcaagcactgcggtggtgtttcctggcgctgctgaggagagcccgcctcgccctacgcagcagcacgagcagcgcctctgctgccgcattggccctacggggtcgcacggtgccctggccctgcaagtgcccctcgccggccgcatggagggcgccgtggaggcagtccgcgacgtgctggcggaggctctgggccgcctgtggcgccagcccgaggcagccggcgcttgcgccggcggccagcagccccaggcccaaccgcagcctgcgccccgccacgaccaccggcaacaggagcagcagcgcgacctcccccgccgtcaggatcatctgcggagcccgccacagcagctcctgctcctgcccgaccagcagcgcccgaacgagggcaaggaggagggggagggtggcgccgtggtgcaggctcgggtgcagcacgtcagccgggctgccgaggcgggtgcggctggccccgcccgcgagggcgtgcgtgtcaacgcgcagcctcctgccgcagagccggcccctgctgccgctgccgcgaagggcagccgctcccgctccggcaaccggggtcgttacggtaacagctcgtcgaacggcatctcgtcctccggcggtggctcgtccggcggttcgtcctgcgagtcgatgcgggcccccagctggaactcccacgtcgaggccgcaggcaagcccgcaacc

SEQ ID NO: 215 - DNA, 723 bp, Chlamydomonas reinhardtii
accggcaagcacgacgcggcaagcacggctgtagtgttccctggagccgcggaggagtcgccgccgcgccccacccaacagcacgagcagcggctgtgctgccgcatcggccccaccggctcgcacggcgcgctggccctgcaggtgcctctggcgggccgcatggagggcgccgtggaggcggtgcgggacgtgttggcggaggcgctgggcaggctgtggcggcagccggaggcggcgggtgcatgcgcaggcggccaacagccgcaggcgcagccgcagccggcacctcgccacgaccaccgccagcaggagcaacagagggatctgccacggcgccaagatcacctgcggtcgccgccgcagcagctgctgctgctgcccgaccagcagcgtcccaatgagggtaaagaggagggggagggtggagccgtcgtgcaggcacgggtgcagcatgtatcccgtgctgctgaggccggagccgcaggccccgcacgtgagggcgtgagggtgaatgcgcagccgccagctgcggagcccgccccggccgccgccgctgccaagggcagccgcagccgcagcggcaacaggggcagatatggcaacagcagcagcaacggcatcagcagcagcggcggcggcagcagcggcggtagcagttgtgagagcatgcgggcgccgtcatggaacagccacgtggaagcggctggcaagccggctaca

SEQ ID NO: 216 - protein, 241 aa, Chlamydomonas reinhardtii
TGKHDAASTAVVFPGAAEESPPRPTQQHEQRLCCRIGPTGSHGALALQVPLAGRMEGAVEAVRDVLAEALGRLWRQPEAAGACAGGQQPQAQPQPAPRHDHRQQEQQRDLPRRQDHLRSPPQQLLLLPDQQRPNEGKEEGEGGAVVQARVQHVSRAAEAGAAGPAREGVRVNAQPPAAEPAPAAAAAKGSRSRSGNRGRYGNSSSNGISSSGGGSSGGSSCESMRAPSWNSHVEAAGKPAT

SEQ ID NO: 217 - DNA, 1395 bp, Chlamydomonas reinhardtii
atgtttgagtttcctgtcgcgcccgtacttgcgcgcaacggtagttccttacgcagcatatggcgtgtattagtatacctaacgtgcctagtcgccgggagagcagccagcggtggagcgcccggccagcaggatgtatcggacttactcgtcgtgtttcccaccttccacaagcgcattggcgtggtggaggcgtcgcgtgcttggcgcatgggtgtgagcacacacatcgtggttgacgacctggtccggctggacgcgctccgggccgcgggctcccgccacaacgagaccttctccgccctgcccgacctgccgggcatgcccaccagcatgcggcacgtgctggcgccgctgctggcgcacaccgccaccggcggccggtacaagtggatgctgctgggtgacgacgacacgctgttctcgctgccggccgtgcgggggctgctgcgggagatgcggctgccgcacacggagccgatcgccatctccgacttcctggtgcattgccgtttcgagcaaagcgccaagctggatgtggccggcggcggcaggcgctacaccgcacccgccacccgggacacgcgctgccccgccgccgccccatcaacacagaccgccgccgccagctcctccaacagcacccctctcatcacgacacccacgacaccggtgccctgcatgctgccgcccgcatcgcggcggccgccccgcttccgcccgcaccccgactgcccgcccgaaggccgcacctccttctacggtggcaccggcgtcattctgtcgctcgggctcatgcagcacctcgcccgcagaagcctgcacgcgcaagcagctctaggaccagactcattatcgtcagcagcagcagctgcggcagccgaggctgctgcggccgacgccgacacgtcattctacgcggtcgccatgtccaactactcaccggcaggggacgtgctcatgagcgaagcgtggcggcgtgccggcatcggcttcacaccgccgccgccactgccctaccagctcgcagtgcaccgcactgcgcagcagcagccctgcgtcgcccccaggcccgacggcgatggtggtggtggtggtggcggcagcgccggcacggcgggccgccccacgcctcctgccgtagtgccgccgcagtgccgccgctttggcagcctggtgggcttcaacgatgaggccagccccgaggccatggtccagcggcaccggctggcggcggaggccgcaccggaggccttccgcggggtggtgtctgcgcacctgcggcagcggcgggccaacaccacggcgttcctggccgccatgcgcgagctgggggcgctgctggcggccggaggggctgctgctgctgcaggcggggcgggggggccgagatga

SEQ ID NO: 218 - protein, 464 aa, Chlamydomonas reinhardtii
MFEFPVAPVLARNGSSLRSIWRVLVYLTCLVAGRAASGGAPGQQDVSDLLVVFPTFHKRIGVVEASRAWRMGVSTHIVVDDLVRLDALRAAGSRHNETFSALPDLPGMPTSMRHVLAPLLAHTATGGRYKWMLLGDDDTLFSLPAVRGLLREMRLPHTEPIAISDFLVHCRFEQSAKLDVAGGGRRYTAPATRDTRCPAAAPSTQTAAASSSNSTPLITTPTTPVPCMLPPASRRPPRFRPHPDCPPEGRTSFYGGTGVILSLGLMQHLARRSLHAQAALGPDSLSSAAAAAAAEAAAADADTSFYAVAMSNYSPAGDVLMSEAWRRAGIGFTPPPPLPYQLAVHRTAQQQPCVAPRPDGDGGGGGGGSAGTAGRPTPPAVVPPQCRRFGSLVGFNDEASPEAMVQRHRLAAEAAPEAFRGVVSAHLRQRRANTTAFLAAMRELGALLAAGGAAAAAGGAGGPR

SEQ ID NO: 219 - DNA, 1416 bp, artificial sequence
  codon optimized
catatgctcgagtttgagtttccagtcgcacctgtgctggctcgcaacggttcctccctgcgctcgatctggcgcgtgctggtgtacctgacctgcctcgtggcaggccgcgcggccagcggtggcgcgccggggcagcaggacgtgagcgacctcctggtggtgttccccactttccacaagcgcatcggcgtcgtggaggcatcccgcgcctggcgcatgggcgtgtccacacacatcgtcgtggacgacctggtgcgcctggacgcgctgcgcgccgccggctcccgccacaacgagacattctccgcgctgccggacctgccggggatgccgacctcgatgcgccacgtgctcgcgcccctgctggcccacaccgcgactgggggccgctacaagtggatgctgctcggcgacgacgacaccctcttctcgctccccgctgtgcggggcctgctgcgtgagatgcgcctgccgcacacggagcctatcgctatctccgacttcctggtgcactgccgcttcgagcagagcgcgaagctggatgtggccggcggcggccgccgctacacagcgcccgctacccgcgacactcgctgcccagcagcagccccgtccacacagacagccgcagcgagctccagcaactcgacgccgctgatcaccaccccgaccacgcccgtgccctgtatgctgcctcccgcgtcgcgtcgccccccacgcttccggccccacccggactgtccgccggagggccgcaccagcttctacggcggcaccggcgtgattctgagcctcggcctgatgcagcatctggcacgccgctccctgcacgcacaggccgcgctgggccccgacagcctgagctcggcagccgcagccgcggcggcggaggcagctgcggcggacgccgacacctccttctatgcggtcgcgatgagcaactacagccccgcaggcgacgtgctcatgtccgaggcatggcggcgggcaggcatcggctttaccccccctccgcccctcccctaccagctggccgtgcatcgcaccgcgcagcagcaaccctgcgtggcaccgcggcctgacggcgacggtggcggcggtggtgggggctcggcgggcacagcgggccgcccaacccctccggctgtggtgcctccgcagtgccgccgcttcggcagcctggtgggtttcaacgacgaggccagcccggaggcgatggtgcagcgccaccgcctggcagccgaggctgcccccgaggcgtttcggggcgtcgtgtccgcccacctgcgccagcgccgcgccaatacgacggcgttcctggccgcgatgcgcgagctgggcgcgctgctggctgccggcggcgctgccgcagccgcgggcggtgctgggggccctcgcaccggttaaggatcc

SEQ ID NO: 220 - DNA, 1389 bp, artificial sequence
  codon optimized
tttgagtttccagtcgcacctgtgctggctcgcaacggttcctccctgcgctcgatctggcgcgtgctggtgtacctgacctgcctcgtggcaggccgcgcggccagcggtggcgcgccggggcagcaggacgtgagcgacctcctggtggtgttccccactttccacaagcgcatcggcgtcgtggaggcatcccgcgcctggcgcatgggcgtgtccacacacatcgtcgtggacgacctggtgcgcctggacgcgctgcgcgccgccggctcccgccacaacgagacattctccgcgctgccggacctgccggggatgccgacctcgatgcgccacgtgctcgcgcccctgctggcccacaccgcgactgggggccgctacaagtggatgctgctcggcgacgacgacaccctcttctcgctccccgctgtgcggggcctgctgcgtgagatgcgcctgccgcacacggagcctatcgctatctccgacttcctggtgcactgccgcttcgagcagagcgcgaagctggatgtggccggcggcggccgccgctacacagcgcccgctacccgcgacactcgctgcccagcagcagccccgtccacacagacagccgcagcgagctccagcaactcgacgccgctgatcaccaccccgaccacgcccgtgccctgtatgctgcctcccgcgtcgcgtcgccccccacgcttccggccccacccggactgtccgccggagggccgcaccagcttctacggcggcaccggcgtgattctgagcctcggcctgatgcagcatctggcacgccgctccctgcacgcacaggccgcgctgggccccgacagcctgagctcggcagccgcagccgcggcggcggaggcagctgcggcggacgccgacacctccttctatgcggtcgcgatgagcaactacagccccgcaggcgacgtgctcatgtccgaggcatggcggcgggcaggcatcggctttaccccccctccgcccctcccctaccagctggccgtgcatcgcaccgcgcagcagcaaccctgcgtggcaccgcggcctgacggcgacggtggcggcggtggtgggggctcggcgggcacagcgggccgcccaacccctccggctgtggtgcctccgcagtgccgccgcttcggcagcctggtgggtttcaacgacgaggccagcccggaggcgatggtgcagcgccaccgcctggcagccgaggctgcccccgaggcgtttcggggcgtcgtgtccgcccacctgcgccagcgccgcgccaatacgacggcgttcctggccgcgatgcgcgagctgggcgcgctgctggctgccggcggcgctgccgcagccgcgggcggtgctgggggccctcgc

SEQ ID NO: 221 - DNA, 1389 bp, Chlamydomonas reinhardtii
tttgagtttcctgtcgcgcccgtacttgcgcgcaacggtagttccttacgcagcatatggcgtgtattagtatacctaacgtgcctagtcgccgggagagcagccagcggtggagcgcccggccagcaggatgtatcggacttactcgtcgtgtttcccaccttccacaagcgcattggcgtggtggaggcgtcgcgtgcttggcgcatgggtgtgagcacacacatcgtggttgacgacctggtccggctggacgcgctccgggccgcgggctcccgccacaacgagaccttctccgccctgcccgacctgccgggcatgcccaccagcatgcggcacgtgctggcgccgctgctggcgcacaccgccaccggcggccggtacaagtggatgctgctgggtgacgacgacacgctgttctcgctgccggccgtgcgggggctgctgcgggagatgcggctgccgcacacggagccgatcgccatctccgacttcctggtgcattgccgtttcgagcaaagcgccaagctggatgtggccggcggcggcaggcgctacaccgcacccgccacccgggacacgcgctgccccgccgccgccccatcaacacagaccgccgccgccagctcctccaacagcacccctctcatcacgacacccacgacaccggtgccctgcatgctgccgcccgcatcgcggcggccgccccgcttccgcccgcaccccgactgcccgcccgaaggccgcacctccttctacggtggcaccggcgtcattctgtcgctcgggctcatgcagcacctcgcccgcagaagcctgcacgcgcaagcagctctaggaccagactcattatcgtcagcagcagcagctgcggcagccgaggctgctgcggccgacgccgacacgtcattctacgcggtcgccatgtccaactactcaccggcaggggacgtgctcatgagcgaagcgtggcggcgtgccggcatcggcttcacaccgccgccgccactgccctaccagctcgcagtgcaccgcactgcgcagcagcagccctgcgtcgcccccaggcccgacggcgatggtggtggtggtggtggcggcagcgccggcacggcgggccgccccacgcctcctgccgtagtgccgccgcagtgccgccgctttggcagcctggtgggcttcaacgatgaggccagccccgaggccatggtccagcggcaccggctggcggcggaggccgcaccggaggccttccgcggggtggtgtctgcgcacctgcggcagcggcgggccaacaccacggcgttcctggccgccatgcgcgagctgggggcgctgctggcggccggaggggctgctgctgctgcaggcggggcgggggggccgaga

SEQ ID NO: 222 - protein, 463 aa, Chlamydomonas reinhardtii
FEFPVAPVLARNGSSLRSIWRVLVYLTCLVAGRAASGGAPGQQDVSDLLVVFPTFHKRIGVVEASRAWRMGVSTHIVVDDLVRLDALRAAGSRHNETFSALPDLPGMPTSMRHVLAPLLAHTATGGRYKWMLLGDDDTLFSLPAVRGLLREMRLPHTEPIAISDFLVHCRFEQSAKLDVAGGGRRYTAPATRDTRCPAAAPSTQTAAASSSNSTPLITTPTTPVPCMLPPASRRPPRFRPHPDCPPEGRTSFYGGTGVILSLGLMQHLARRSLHAQAALGPDSLSSAAAAAAAEAAAADADTSFYAVAMSNYSPAGDVLMSEAWRRAGIGFTPPPPLPYQLAVHRTAQQQPCVAPRPDGDGGGGGGGSAGTAGRPTPPAVVPPQCRRFGSLVGFNDEASPEAMVQRHRLAAEAAPEAFRGVVSAHLRQRRANTTAFLAAMRELGALLAAGGAAAAAGGAGGPR

SEQ ID NO: 223 - DNA, 1947 bp, Chlamydomonas reinhardtii
atgactgtatatgtccaccttgcactaaagttattcctactgttccgctcaagctgcccatcacctggcgccaaccgcatgttgcttgtgcactgttcgcatgcacggaccccttcgcgtgttcaggaggtgctgtggaagcctgctcctcgcggctccaaggagtgccccgacaactgcaacaacgtggggcgctgcaactacgacaccggctactgcgactgcgcggcggggtggacgggcgtgggctgcaagacgccccagaagcggccgtgcaccagtacctggagccgatgcggcggcatttgtgacgacgacctcgccatctgctactgtgacggccagggccccaaccagttcggtcgcatccccgcgcccccgggctcgccacccggaaccccgcccattcgtgtgggcaggccgctggtcaccgagtacatggcgccaaacgagacgtgggacggcaagtgggcgttcggcaggcagccgtactccaatgtgtacggcccgcaaggatactgcaacgtcagcaagccggtgtgggcggcggtgtgcagcatggacgcgctggccgggcccacctgtgacgagccgctggagtcgttctgccctggcgcctgcagcgggcatggccgctgctacctgggctactgctactgcgacgagggctactacggccacgactgtgcgcggcgcaaggctggactgccgcttctgccaagcagcatccccaccacgccctggctggcgtccgtggtgcgcgagccgccggcagcgcaggagccgccgccggtgcccacccgcaagcggccgctcgtgtacgtgtacgacctggagcccttctaccaggcccgcatcctgcagtacaggtggacggacgagtgggtgtacgcagtcgacaccctgctgcacgagagcttcctgatcagcgagcaccgcacctttgacccggaagaggctgactacttctacgttccccaccaagccacgtgtcttccgtttcccattggccgttgggcggatttcccatggtttggcggcactggaggcgcacggcctcgccagatgatcaatttcatccgggaggtgcacacctggatcgacaccaactaccccttctggaagcggcgccagggccgggaccatatttggacgtggacgcacgatgagggggcctgctgggctcccacggtcctcaacaactcggtttggctgacgcactgggggcgcatggagctcaaccacaagtctcacggccttcccgaggacaactacaacaaggagttcaagtccgtcaaccagcctgaggggtacctggtccacatccagggccatccctgctacaacccccaaaaagacctggtcattccctccttcaagcggcccgagcactaccacaaaagcggcctggttggaaacccgacacgcgaacgagatgtgtccttctacttcaagggagatgtgggcaagggcaggtttccgccgtatagccggggcgtacggcaagggatctacaagctagcaaaggagggcgactgggccaccaagcacaagttcctgatcgggggccgcagcgacgtgccgggcgactacagcgacatgctcagccgcgccatattctgcctcgtggcggcaggtgacggctggtcggggcgcatggaggacgccatgcttcacggttgcatccccgtcatcatcattgacgaagttcacgtcgttttcgagtcccttctagacgtggatactttctccatccgcatcgcacaaaaggatgtaccacgtattctggagatcctgcaagccgttcccgagcgcaaaattcgttccatgcaggcgcacctggggcacgtgtggcacaggtacgtctgggtgggcgatccaagcgtggatgatgcatttgggaccatcctgcagtggctgtattcaaggatcccttacacccggtga

SEQ ID NO: 224 - protein, 648 aa, Chlamydomonas reinhardtii
MTVYVHLALKLFLLFRSSCPSPGANRMLLVHCSHARTPSRVQEVLWKPAPRGSKECPDNCNNVGRCNYDTGYCDCAAGWTGVGCKTPQKRPCTSTWSRCGGICDDDLAICYCDGQGPNQFGRIPAPPGSPPGTPPIRVGRPLVTEYMAPNETWDGKWAFGRQPYSNVYGPQGYCNVSKPVWAAVCSMDALAGPTCDEPLESFCPGACSGHGRCYLGYCYCDEGYYGHDCARRKAGLPLLPSSIPTTPWLASVVREPPAAQEPPPVPTRKRPLVYVYDLEPFYQARILQYRWTDEWVYAVDTLLHESFLISEHRTFDPEEADYFYVPHQATCLPFPIGRWADFPWFGGTGGARPRQMINFIREVHTWIDTNYPFWKRRQGRDHIWTWTHDEGACWAPTVLNNSVWLTHWGRMELNHKSHGLPEDNYNKEFKSVNQPEGYLVHIQGHPCYNPQKDLVIPSFKRPEHYHKSGLVGNPTRERDVSFYFKGDVGKGRFPPYSRGVRQGIYKLAKEGDWATKHKFLIGGRSDVPGDYSDMLSRAIFCLVAAGDGWSGRMEDAMLHGCIPVIIIDEVHVVFESLLDVDTFSIRIAQKDVPRILEILQAVPERKIRSMQAHLGHVWHRYVWVGDPSVDDAFGTILQWLYSRIPYTR

SEQ ID NO: 225 - DNA, 1968 bp, artificial sequence
  codon optimized
catatgctcgagacggtgtatgtgcatctcgctctgaagctgtttctgctgtttcgttcctcctgcccttcgcccggtgctaaccggatgctgctcgtgcactgctcgcacgcccgcaccccatcgcgcgtgcaggaggtgctctggaagcccgcaccgcgcggtagcaaggagtgccctgacaactgcaacaacgtggggcgctgcaactacgacactggttactgcgactgcgcggctggttggacgggcgtgggctgcaagacaccgcagaagcgtccctgtacatcgacctggtcccgctgcggtggcatctgcgacgacgacctggccatctgctactgtgacggccagggccctaatcagttcggccggattccggcgccaccgggttcgccgcctgggactccccctatccgcgtgggccgtcccctggtgactgagtacatggcgcctaacgagacgtgggacggcaagtgggcgttcggtcgccagccctactccaacgtctatggcccacagggctactgcaacgtgagcaagccggtgtgggcagcggtgtgctcgatggacgcgctggcgggtcccacgtgcgatgagcctctggagagcttctgccctggtgcctgctccggccacggtcgctgctatctgggctattgctactgcgacgagggctactacggccacgactgcgctcgccgcaaggccgggctccccctgctgccctcgtcgatcccgacaactccgtggctggcctcggtggtgcgggagcctccagctgcccaggagccaccgcccgtgccaacgcgcaagcgcccgctggtgtacgtgtatgacctggagcccttctaccaggcgcgcatcctgcaataccgctggaccgacgagtgggtgtacgccgtcgataccctgctgcacgagagcttcctgatctcggagcatcggaccttcgaccccgaggaggcagactacttctacgtgccccaccaagcgacctgcctgccttttccgatcggccgctgggcggacttcccgtggtttggcggcactggcggcgcccgtccgcgccagatgattaactttatccgcgaggtgcacacgtggatcgacaccaactacccgttttggaagcgccggcagggccgcgaccacatttggacttggacccatgacgagggcgcctgctgggctcccacggtgctgaacaacagcgtgtggctgacccattgggggcgcatggagctgaaccacaagagccacggcctgcccgaggataactacaacaaggagttcaagagcgtgaaccagcctgagggctacctggtgcacatccagggccatccttgctacaatccccagaaggacctcgtgatcccctcgtttaagcggcccgagcactaccacaagtcggggctcgtgggcaacccaacccgtgagcgcgacgtgagcttctacttcaagggcgacgtgggcaagggccgcttcccgccctactcgcgcggtgtgcgccagggcatctacaagctggcgaaggagggggactgggcaaccaagcacaagttcctcattggcggccgtagcgatgtgcccggcgattactccgacatgctgtcgcgggccatcttctgcctggtggctgcgggcgacggctggagcggccgtatggaggatgccatgctgcacggctgcatccccgtcatcatcatcgacgaggtgcatgtggtgttcgagagcctgctggacgtggacaccttcagcatccgcattgctcaaaaggacgtgccccgcatcctggagatcctccaggctgtgccggagcgcaagatccgctccatgcaagcccacctgggccacgtgtggcaccgctacgtgtgggtcggggacccctcggtggacgatgcgttcggcaccatcctccagtggctctacagccgcattccgtacacccggaccggttaaggatcc

SEQ ID NO: 226 - DNA, 1941 bp, artificial sequence
  codon optimized
acggtgtatgtgcatctcgctctgaagctgtttctgctgtttcgttcctcctgcccttcgcccggtgctaaccggatgctgctcgtgcactgctcgcacgcccgcaccccatcgcgcgtgcaggaggtgctctggaagcccgcaccgcgcggtagcaaggagtgccctgacaactgcaacaacgtggggcgctgcaactacgacactggttactgcgactgcgcggctggttggacgggcgtgggctgcaagacaccgcagaagcgtccctgtacatcgacctggtcccgctgcggtggcatctgcgacgacgacctggccatctgctactgtgacggccagggccctaatcagttcggccggattccggcgccaccgggttcgccgcctgggactccccctatccgcgtgggccgtcccctggtgactgagtacatggcgcctaacgagacgtgggacggcaagtgggcgttcggtcgccagccctactccaacgtctatggcccacagggctactgcaacgtgagcaagccggtgtgggcagcggtgtgctcgatggacgcgctggcgggtcccacgtgcgatgagcctctggagagcttctgccctggtgcctgctccggccacggtcgctgctatctgggctattgctactgcgacgagggctactacggccacgactgcgctcgccgcaaggccgggctccccctgctgccctcgtcgatcccgacaactccgtggctggcctcggtggtgcgggagcctccagctgcccaggagccaccgcccgtgccaacgcgcaagcgcccgctggtgtacgtgtatgacctggagcccttctaccaggcgcgcatcctgcaataccgctggaccgacgagtgggtgtacgccgtcgataccctgctgcacgagagcttcctgatctcggagcatcggaccttcgaccccgaggaggcagactacttctacgtgccccaccaagcgacctgcctgccttttccgatcggccgctgggcggacttcccgtggtttggcggcactggcggcgcccgtccgcgccagatgattaactttatccgcgaggtgcacacgtggatcgacaccaactacccgttttggaagcgccggcagggccgcgaccacatttggacttggacccatgacgagggcgcctgctgggctcccacggtgctgaacaacagcgtgtggctgacccattgggggcgcatggagctgaaccacaagagccacggcctgcccgaggataactacaacaaggagttcaagagcgtgaaccagcctgagggctacctggtgcacatccagggccatccttgctacaatccccagaaggacctcgtgatcccctcgtttaagcggcccgagcactaccacaagtcggggctcgtgggcaacccaacccgtgagcgcgacgtgagcttctacttcaagggcgacgtgggcaagggccgcttcccgccctactcgcgcggtgtgcgccagggcatctacaagctggcgaaggagggggactgggcaaccaagcacaagttcctcattggcggccgtagcgatgtgcccggcgattactccgacatgctgtcgcgggccatcttctgcctggtggctgcgggcgacggctggagcggccgtatggaggatgccatgctgcacggctgcatccccgtcatcatcatcgacgaggtgcatgtggtgttcgagagcctgctggacgtggacaccttcagcatccgcattgctcaaaaggacgtgccccgcatcctggagatcctccaggctgtgccggagcgcaagatccgctccatgcaagcccacctgggccacgtgtggcaccgctacgtgtgggtcggggacccctcggtggacgatgcgttcggcaccatcctccagtggctctacagccgcattccgtacacccgg

SEQ ID NO: 227 - DNA, 1941 bp, Chlamydomonas reinhardtii
actgtatatgtccaccttgcactaaagttattcctactgttccgctcaagctgcccatcacctggcgccaaccgcatgttgcttgtgcactgttcgcatgcacggaccccttcgcgtgttcaggaggtgctgtggaagcctgctcctcgcggctccaaggagtgccccgacaactgcaacaacgtggggcgctgcaactacgacaccggctactgcgactgcgcggcggggtggacgggcgtgggctgcaagacgccccagaagcggccgtgcaccagtacctggagccgatgcggcggcatttgtgacgacgacctcgccatctgctactgtgacggccagggccccaaccagttcggtcgcatccccgcgcccccgggctcgccacccggaaccccgcccattcgtgtgggcaggccgctggtcaccgagtacatggcgccaaacgagacgtgggacggcaagtgggcgttcggcaggcagccgtactccaatgtgtacggcccgcaaggatactgcaacgtcagcaagccggtgtgggcggcggtgtgcagcatggacgcgctggccgggcccacctgtgacgagccgctggagtcgttctgccctggcgcctgcagcgggcatggccgctgctacctgggctactgctactgcgacgagggctactacggccacgactgtgcgcggcgcaaggctggactgccgcttctgccaagcagcatccccaccacgccctggctggcgtccgtggtgcgcgagccgccggcagcgcaggagccgccgccggtgcccacccgcaagcggccgctcgtgtacgtgtacgacctggagcccttctaccaggcccgcatcctgcagtacaggtggacggacgagtgggtgtacgcagtcgacaccctgctgcacgagagcttcctgatcagcgagcaccgcacctttgacccggaagaggctgactacttctacgttccccaccaagccacgtgtcttccgtttcccattggccgttgggcggatttcccatggtttggcggcactggaggcgcacggcctcgccagatgatcaatttcatccgggaggtgcacacctggatcgacaccaactaccccttctggaagcggcgccagggccgggaccatatttggacgtggacgcacgatgagggggcctgctgggctcccacggtcctcaacaactcggtttggctgacgcactgggggcgcatggagctcaaccacaagtctcacggccttcccgaggacaactacaacaaggagttcaagtccgtcaaccagcctgaggggtacctggtccacatccagggccatccctgctacaacccccaaaaagacctggtcattccctccttcaagcggcccgagcactaccacaaaagcggcctggttggaaacccgacacgcgaacgagatgtgtccttctacttcaagggagatgtgggcaagggcaggtttccgccgtatagccggggcgtacggcaagggatctacaagctagcaaaggagggcgactgggccaccaagcacaagttcctgatcgggggccgcagcgacgtgccgggcgactacagcgacatgctcagccgcgccatattctgcctcgtggcggcaggtgacggctggtcggggcgcatggaggacgccatgcttcacggttgcatccccgtcatcatcattgacgaagttcacgtcgttttcgagtcccttctagacgtggatactttctccatccgcatcgcacaaaaggatgtaccacgtattctggagatcctgcaagccgttcccgagcgcaaaattcgttccatgcaggcgcacctggggcacgtgtggcacaggtacgtctgggtgggcgatccaagcgtggatgatgcatttgggaccatcctgcagtggctgtattcaaggatcccttacacccgg

SEQ ID NO: 228 - protein, 647 aa, Chlamydomonas reinhardtii
TVYVHLALKLFLLFRSSCPSPGANRMLLVHCSHARTPSRVQEVLWKPAPRGSKECPDNCNNVGRCNYDTGYCDCAAGWTGVGCKTPQKRPCTSTWSRCGGICDDDLAICYCDGQGPNQFGRIPAPPGSPPGTPPIRVGRPLVTEYMAPNETWDGKWAFGRQPYSNVYGPQGYCNVSKPVWAAVCSMDALAGPTCDEPLESFCPGACSGHGRCYLGYCYCDEGYYGHDCARRKAGLPLLPSSIPTTPWLASVVREPPAAQEPPPVPTRKRPLVYVYDLEPFYQARILQYRWTDEWVYAVDTLLHESFLISEHRTFDPEEADYFYVPHQATCLPFPIGRWADFPWFGGTGGARPRQMINFIREVHTWIDTNYPFWKRRQGRDHIWTWTHDEGACWAPTVLNNSVWLTHWGRMELNHKSHGLPEDNYNKEFKSVNQPEGYLVHIQGHPCYNPQKDLVIPSFKRPEHYHKSGLVGNPTRERDVSFYFKGDVGKGRFPPYSRGVRQGIYKLAKEGDWATKHKFLIGGRSDVPGDYSDMLSRAIFCLVAAGDGWSGRMEDAMLHGCIPVIIIDEVHVVFESLLDVDTFSIRIAQKDVPRILEILQAVPERKIRSMQAHLGHVWHRYVWVGDPSVDDAFGTILQWLYSRIPYTR

SEQ ID NO: 229 - DNA, 273 bp, Chlamydomonas reinhardtii
atgccttcaaggtttcttggacgtgccgacgcagggagctattacgatattttgggtgtggagcagactgctgcatcggaggaaatcagggcggcataccggtcgctggcgaaggcgttccacccggacgtcagccaagaggactcgcacgaggtgtttgcggagatcaacagcgcctacgctgtgctgtcagacccggaggagcgcggccgctatgactacctgtggcgctatgagcaggtgcgcgtgtgtgcgtgtgtgcgggagagatag

SEQ ID NO: 230 - protein, 90 aa, Chlamydomonas reinhardtii
MPSRFLGRADAGSYYDILGVEQTAASEEIRAAYRSLAKAFHPDVSQEDSHEVFAEINSAYAVLSDPEERGRYDYLWRYEQVRVCACVRER

SEQ ID NO: 231 - DNA, 288 bp, artificial sequence
  codon optimized
catatgctcgagccaagccgttttctgggtcgcgccgacgcaggtagctattatgatattctgggcgtcgagcagacggcggcctccgaggagatccgcgctgcctaccgcagcctggcaaaggcgttccacccggacgtgtcgcaggaggacagccacgaggtgttcgcggagatcaactcggcgtacgctgtgctgtcggaccccgaggagcgcggtcgctacgactacctgtggcgttacgagcaggtccgggtctgcgcgtgcgtccgtgagcgctagggatcc

SEQ ID NO: 232 - DNA, 267 bp, artificial sequence
  codon optimized
ccaagccgttttctgggtcgcgccgacgcaggtagctattatgatattctgggcgtcgagcagacggcggcctccgaggagatccgcgctgcctaccgcagcctggcaaaggcgttccacccggacgtgtcgcaggaggacagccacgaggtgttcgcggagatcaactcggcgtacgctgtgctgtcggaccccgaggagcgcggtcgctacgactacctgtggcgttacgagcaggtccgggtctgcgcgtgcgtccgtgagcgc

SEQ ID NO: 233 - DNA, 267 bp, Chlamydomonas reinhardtii
ccttcaaggtttcttggacgtgccgacgcagggagctattacgatattttgggtgtggagcagactgctgcatcggaggaaatcagggcggcataccggtcgctggcgaaggcgttccacccggacgtcagccaagaggactcgcacgaggtgtttgcggagatcaacagcgcctacgctgtgctgtcagacccggaggagcgcggccgctatgactacctgtggcgctatgagcaggtgcgcgtgtgtgcgtgtgtgcgggagaga

SEQ ID NO: 234 - protein, 89 aa, Chlamydomonas reinhardtii
PSRFLGRADAGSYYDILGVEQTAASEEIRAAYRSLAKAFHPDVSQEDSHEVFAEINSAYAVLSDPEERGRYDYLWRYEQVRVCACVRER

SEQ ID NO: 235 - DNA, 1080 bp, Chlamydomonas reinhardtii
atggtgtatacaagggcgtcgcgggcgccggccggggccctactccttgccctcggcatcgtgtgcattgcggcactcagcatgcgaggtgctgacgcccgtcctggtcccggcggcaaggacctgttcaatggcactctccccgacttcaacggcacccaccctgacttcaacggcacccgccccgactgcaatggcacccgcaatggcacccgccctgacttcaatggcaccaagcttgagggcggtcgcggcggctcccaccgtggtctgcgtggcggcggcgaccgtggcggtcgtgatggcgacttcaacggcacccatcctgagtttaatggcactcgccccgacttcaacggcacccaccttgacttcaacggcacccgccccgactgcaatggcacccgcaatggcacccgccctgacttcaatggcaccaagcccgagggcggccgcggcggcttccaccgtggtctgcgtggcggcggcgaccgtggcggtcgtgatggcgacttcaacggcacccatcctgagtttaatggcactcgccccgacttcaacggcacccaccttgacttcgacggcacccgccccgactgcaatggcacccgcaatggcacccgccctgacttcaatggcaccaagcccgagggcggccgcggcggctcccaccgtggtctgcgtggcggcggcgaccgtggcggtcatgatggcgacttcaacggcacccatcctgagtttaatggcactcgccccgacttcaacggcacccaccctgacttcaacggcacccgccccgactgcaatggcacccgcgacggcaccctccctgacttcaatggcaccaagcccgagggcggccgcggcggctcccaccgtggtctgcgcggtggcggcggcggcggcggcagctccaccgacagcggcagcagcagcccgcgtccctcccgcccgcctcgcccagctgatctggccatgccgtcgcgccctgagcatggctcggtgccggcttcctctcgcccgcctcggcccccgggcgcgaagaccgccaggcctcccaagccgtcagcacggcggtag

SEQ ID NO: 236 - protein, 359 aa, Chlamydomonas reinhardtii
MVYTRASRAPAGALLLALGIVCIAALSMRGADARPGPGGKDLFNGTLPDFNGTHPDFNGTRPDCNGTRNGTRPDFNGTKLEGGRGGSHRGLRGGGDRGGRDGDFNGTHPEFNGTRPDFNGTHLDFNGTRPDCNGTRNGTRPDFNGTKPEGGRGGFHRGLRGGGDRGGRDGDFNGTHPEFNGTRPDFNGTHLDFDGTRPDCNGTRNGTRPDFNGTKPEGGRGGSHRGLRGGGDRGGHDGDFNGTHPEFNGTRPDFNGTHPDFNGTRPDCNGTRDGTLPDFNGTKPEGGRGGSHRGLRGGGGGGGSSTDSGSSSPRPSRPPRPADLAMPSRPEHGSVPASSRPPRPPGAKTARPPKPSARR

SEQ ID NO: 237 - DNA, 1101 bp, artificial sequence
  codon optimized
catatgctcgaggtgtacacacgcgccagccgtgcccctgccggcgccctgctgctggcactgggcatcgtgtgcatcgctgccctgtccatgcgtggcgctgacgcccgcccgggcccaggcgggaaggacctgttcaacggcacgctgcctgacttcaacggtacgcaccccgactttaacggcactcgcccggattgcaatggcacccgcaatggcacccgccccgacttcaacggcaccaagctggagggcggtcggggtggtagccaccgcggcctgcgcggtggcggcgaccgtggcggtcgcgacggtgacttcaacggcacgcaccctgagttcaacggcacccggccagacttcaacggcacccacctggacttcaacggcacccggcccgactgcaacggcacgcgcaatggcacgcgtccggacttcaacggcaccaagcccgaggggggtcgtggcggcttccaccggggcctccggggtggcggcgaccgcggtggccgcgacggcgacttcaacggcacccacccggagttcaatggcactcgccccgacttcaacggcactcacctggacttcgacgggacccgcccggactgcaacgggacgcgcaacggcactcgcccggatttcaacggcaccaagccggagggtggccgtggcggctcgcaccgcggtctgcgcggcggcggcgaccgcggtggccacgatggggacttcaacgggacccaccccgagttcaacggcacccgtcccgacttcaatggcacgcatcccgacttcaacggcactcgtccggactgcaacggcacacgcgacggcacgctgcctgatttcaacggcactaagcctgagggtggtcgcgggggctcgcaccgtggcctccgtggcggtggcggtggtgggggctcgtccaccgactccggcagcagcagccctcgcccctcgcgccctccccgccctgcggacctggccatgccctcgcgcccggagcacggctccgtgcccgccagcagccgtcccccacgccctcccggtgccaagaccgctcgtccgccgaagccctcggcccgtcggaccggttaaggatcc

SEQ ID NO: 238 - DNA, 1074 bp, artificial sequence
  codon optimized
gtgtacacacgcgccagccgtgcccctgccggcgccctgctgctggcactgggcatcgtgtgcatcgctgccctgtccatgcgtggcgctgacgcccgcccgggcccaggcgggaaggacctgttcaacggcacgctgcctgacttcaacggtacgcaccccgactttaacggcactcgcccggattgcaatggcacccgcaatggcacccgccccgacttcaacggcaccaagctggagggcggtcggggtggtagccaccgcggcctgcgcggtggcggcgaccgtggcggtcgcgacggtgacttcaacggcacgcaccctgagttcaacggcacccggccagacttcaacggcacccacctggacttcaacggcacccggcccgactgcaacggcacgcgcaatggcacgcgtccggacttcaacggcaccaagcccgaggggggtcgtggcggcttccaccggggcctccggggtggcggcgaccgcggtggccgcgacggcgacttcaacggcacccacccggagttcaatggcactcgccccgacttcaacggcactcacctggacttcgacgggacccgcccggactgcaacgggacgcgcaacggcactcgcccggatttcaacggcaccaagccggagggtggccgtggcggctcgcaccgcggtctgcgcggcggcggcgaccgcggtggccacgatggggacttcaacgggacccaccccgagttcaacggcacccgtcccgacttcaatggcacgcatcccgacttcaacggcactcgtccggactgcaacggcacacgcgacggcacgctgcctgatttcaacggcactaagcctgagggtggtcgcgggggctcgcaccgtggcctccgtggcggtggcggtggtgggggctcgtccaccgactccggcagcagcagccctcgcccctcgcgccctccccgccctgcggacctggccatgccctcgcgcccggagcacggctccgtgcccgccagcagccgtcccccacgccctcccggtgccaagaccgctcgtccgccgaagccctcggcccgtcgg

SEQ ID NO: 239 - DNA, 1074 bp, Chlamydomonas reinhardtii
gtgtatacaagggcgtcgcgggcgccggccggggccctactccttgccctcggcatcgtgtgcattgcggcactcagcatgcgaggtgctgacgcccgtcctggtcccggcggcaaggacctgttcaatggcactctccccgacttcaacggcacccaccctgacttcaacggcacccgccccgactgcaatggcacccgcaatggcacccgccctgacttcaatggcaccaagcttgagggcggtcgcggcggctcccaccgtggtctgcgtggcggcggcgaccgtggcggtcgtgatggcgacttcaacggcacccatcctgagtttaatggcactcgccccgacttcaacggcacccaccttgacttcaacggcacccgccccgactgcaatggcacccgcaatggcacccgccctgacttcaatggcaccaagcccgagggcggccgcggcggcttccaccgtggtctgcgtggcggcggcgaccgtggcggtcgtgatggcgacttcaacggcacccatcctgagtttaatggcactcgccccgacttcaacggcacccaccttgacttcgacggcacccgccccgactgcaatggcacccgcaatggcacccgccctgacttcaatggcaccaagcccgagggcggccgcggcggctcccaccgtggtctgcgtggcggcggcgaccgtggcggtcatgatggcgacttcaacggcacccatcctgagtttaatggcactcgccccgacttcaacggcacccaccctgacttcaacggcacccgccccgactgcaatggcacccgcgacggcaccctccctgacttcaatggcaccaagcccgagggcggccgcggcggctcccaccgtggtctgcgcggtggcggcggcggcggcggcagctccaccgacagcggcagcagcagcccgcgtccctcccgcccgcctcgcccagctgatctggccatgccgtcgcgccctgagcatggctcggtgccggcttcctctcgcccgcctcggcccccgggcgcgaagaccgccaggcctcccaagccgtcagcacggcgg

SEQ ID NO: 240 - protein, 358 aa, Chlamydomonas reinhardtii
VYTRASRAPAGALLLALGIVCIAALSMRGADARPGPGGKDLFNGTLPDFNGTHPDFNGTRPDCNGTRNGTRPDFNGTKLEGGRGGSHRGLRGGGDRGGRDGDFNGTHPEFNGTRPDFNGTHLDFNGTRPDCNGTRNGTRPDFNGTKPEGGRGGFHRGLRGGGDRGGRDGDFNGTHPEFNGTRPDFNGTHLDFDGTRPDCNGTRNGTRPDFNGTKPEGGRGGSHRGLRGGGDRGGHDGDFNGTHPEFNGTRPDFNGTHPDFNGTRPDCNGTRDGTLPDFNGTKPEGGRGGSHRGLRGGGGGGGSSTDSGSSSPRPSRPPRPADLAMPSRPEHGSVPASSRPPRPPGAKTARPPKPSARR

SEQ ID NO: 241 - DNA, 3444 bp, Chlamydomonas reinhardtii
atgcggccgacggcaccgccgccaatctcaaccaccggcggcggcggcggcggctgctcggcggcggcggcactgcctgcggcggcgccgcatgacgccgcccagacgcacggcgctggagctgtagcaggcggcggcggcgcgggagtcggcagtagcagcagcggcagtggcggtaacggcggcagtagcggcggcagtagcagctcggctggcggtggcagtggcggtggcggcggcggcggtcccaccactccgccggcatcgacgccgccgccgtcgagtcgcccctcggtgccgccaacgcaacagcaacagcaacagcaacagcaacagcaacagcaacagcaacagcaacagcaacagcaacagcagccggcgccggggactagtgcagggcagcgcgcgccaacggcggcggcgggactcgcccctgatgactcaccgcaggcacgacccacatccgtacccgcacccgcccgcagcagcggcggtgctacagccaccaccacctctggctccaccttccccactgcccttgcccctgccgccggagccattggcggcagcggcggcaacagaaccgccggggcggaaggtctcggtggcagtagcacggccggcggcggcggccccgacggttcccctcagccgcagcgcccgccgccgcaagagcagcagcagccacagcggccgcagcaacagaagcaacagcagccggagccgtcgccgccacaggagcagccacgccagcccgccgccgccgccgagcaaccggacgcggcgcggcagatcctgaacgagctactgttgagccgcggcggcggcggcggcggcggcggcaatgcaggcggcggcggcggcggcgacggtccagccaccggcgacagtgacatggcaactgccgccgcgccgtctgctcttcatggcgctattggcgccggcgctggcgcttctgggccggcagcggcgggccctgcgggcagtggcggcggcggcggcagcggcggcggcggcggcggcggccagctgccgccaagcggcgaggcggagacggcgcgggggacactgcggtggcggctggactcggcgggtggcggcgagctgacgtgggatgagcgcgagcaggagtctttggtgcccctggcggcagacttgctacagctctggggccgggtgctactgctgctgctgcgacgcagcctcagcctgcggcaggcggcgccgatgctggtccggctgcgccacaccagcctgcgcctggtacggttggcggtgatgcaggcgaccgcgccggcatgggtggagctgcacgtgctgccgctgcggctgcgactggtccagctgcaggtggtgggggcggcgcagtatggccctcctggttcggcaggtggtgttgctggcatggacgtggatggtggcgccggatcagctgctggtgctggcggcggtggcggcggcctagggctgcccacctcccagctgccgccgcagccgccgcagccgccgcagccgccttccaccgccctggcgccgccgctgcagctgtcccgcgagccctccctgggccactacttcgaccacttgctgccaccagccgtaggcggcggcggcggggcggcggccgcagcaacaggggcagtggcagcggcggcggcgccgccaccgccgcccctggcgccgccggcctggtctggtctggggctgggaccggggccgggtctgggcctgccttctagcggcggcgggcccggcggcggtgcggctgctgctccggagccgccgccgccgccgccacggccaccgccgccgtacgacagcggtacgacctcgccaaccgctggctttattggtggcggcggtggtgctggaggtagagtaggaggcggcgttgatggcggcggtggcggtgcagagggaggcggtcgcagtgggggcggcgacggtgcgcgcggtggtgcagacggaggtgacgtggatgggagtggcggcagtggcggcagcggtggcggcggtagtgtcgccagtggcggcggcggtagcggcagtggcgccagtcgggcactgtccgcctccatccggcaccccggcggcaccaccatcacgctacagctgccgcagggacagcaacagcaacagcaacagcaacagcaacagcaacagcaacagcgacagcaacagcaacagcaacagcaacagcaacagcaacagcaacagaacgccggcggcggcggcggcggcggcggcgataaacccaccgacacggcgctcgagccgccgccgccaccgccatgccaggcgctgcggaccggtgtggcagacaacatgcacgacttcgcaccgccgccgccagccgctacggcgacgggctttgccgccggcggcgttccgccgccgccccggacgccgccgccctctcctatgctggtggacggcggcggcggtgctgctggcggcggcggcagtgggcccggccctgccggcggcggtggcggggctggaggtcccggtggcggtgctgggtacgtgccgtcttcgtcgctcatgcagcaacagcagctgcagcagccacaacaaacgcagcaacaaccgcaaccaaaccccttcagcagcacggcgccgccgccgccgcccgccaccgcgccaacccctgcacccacgccgccagcggctgcaacttgggcggcggcggcggccccgtttccggctacgcagttcccgcctgcggcgcccgcgccctgcacgttgctcagcagctctgcagctgacccctttatcgaggcgccatcgggtagcaacacgcctgtcggaattgggagcggcggcggggctgcgggagagtgggcaggtggtgtgcagcagcatgtgtttcagcagcccctggcggcggggagtggcgggggcgttggtgccggtggtggcggcgaaaagcgcgttgacggcggcggcggcggcggcggcggcgcgttcgtcctgccctcggcggcgttggcggcggcgctgtcgccggcggaggtggcggcagtgcaggcggcggcggcgctcatgcagcgcggcgccgacccggcggtggcgggcctgccgccacaggccgccgccatcgctctgcaggcggcggcggcggaccagcaagccctggcggcggccgccgccgccgccgccggcgctgccggcggtgctgctagtgtcaatggtgctgctggggcgcagttgcctctggcggccccgtctccagcggccccgtcttcagcggctgcttggggcgcagctcctgccgccatggcgcctgcggcgggcccgcctgcgttcctgccatctgctgcggtgacgccgcctggctcgacggccgcaacagggctgctgccgccggaggcggcggctgtgtacccggcggcggcagaggcgccgccgccgccgaccgctttcagtgcaccccagcggtaa

SEQ ID NO: 242 - protein, 1147 aa, Chlamydomonas reinhardtii
MRPTAPPPISTTGGGGGGCSAAAALPAAAPHDAAQTHGAGAVAGGGGAGVGSSSSGSGGNGGSSGGSSSSAGGGSGGGGGGGPTTPPASTPPPSSRPSVPPTQQQQQQQQQQQQQQQQQQQQQQQPAPGTSAGQRAPTAAAGLAPDDSPQARPTSVPAPARSSGGATATTTSGSTFPTALAPAAGAIGGSGGNRTAGAEGLGGSSTAGGGGPDGSPQPQRPPPQEQQQPQRPQQQKQQQPEPSPPQEQPRQPAAAAEQPDAARQILNELLLSRGGGGGGGGNAGGGGGGDGPATGDSDMATAAAPSALHGAIGAGAGASGPAAAGPAGSGGGGGSGGGGGGGQLPPSGEAETARGTLRWRLDSAGGGELTWDEREQESLVPLAADLLQLWGRVLLLLLRRSLSLRQAAPMLVRLRHTSLRLVRLAVMQATAPAWVELHVLPLRLRLVQLQVVGAAQYGPPGSAGGVAGMDVDGGAGSAAGAGGGGGGLGLPTSQLPPQPPQPPQPPSTALAPPLQLSREPSLGHYFDHLLPPAVGGGGGAAAAATGAVAAAAAPPPPPLAPPAWSGLGLGPGPGLGLPSSGGGPGGGAAAAPEPPPPPPRPPPPYDSGTTSPTAGFIGGGGGAGGRVGGGVDGGGGGAEGGGRSGGGDGARGGADGGDVDGSGGSGGSGGGGSVASGGGGSGSGASRALSASIRHPGGTTITLQLPQGQQQQQQQQQQQQQQQRQQQQQQQQQQQQQQQNAGGGGGGGGDKPTDTALEPPPPPPCQALRTGVADNMHDFAPPPPAATATGFAAGGVPPPPRTPPPSPMLVDGGGGAAGGGGSGPGPAGGGGGAGGPGGGAGYVPSSSLMQQQQLQQPQQTQQQPQPNPFSSTAPPPPPATAPTPAPTPPAAATWAAAAAPFPATQFPPAAPAPCTLLSSSAADPFIEAPSGSNTPVGIGSGGGAAGEWAGGVQQHVFQQPLAAGSGGGVGAGGGGEKRVDGGGGGGGGAFVLPSAALAAALSPAEVAAVQAAAALMQRGADPAVAGLPPQAAAIALQAAAADQQALAAAAAAAAGAAGGAASVNGAAGAQLPLAAPSPAAPSSAAAWGAAPAAMAPAAGPPAFLPSAAVTPPGSTAATGLLPPEAAAVYPAAAEAPPPPTAFSAPQR

SEQ ID NO: 243 - DNA, 3459 bp, artificial sequence
  codon optimized
catatgctcgagcgtccgacggcacctccccctattagcaccactggcggtgggggtggcggttgctccgcagcagcggcgctccctgcggctgcgccccacgatgctgctcagacccacggcgcaggcgccgtggctggcggtggcggcgcaggggtgggcagcagcagctcgggcagcggcggcaacggtggtagctcgggcggctccagcagcagcgccggtggcggttcgggtggcggtggcgggggtggccccaccaccccaccagcgtccacgccacccccaagctcgcgcccgagcgtccctccgacccagcagcagcagcagcaacagcagcagcagcagcagcagcaacagcagcaacagcagcagcagcagcagccggcacccggcacctcggcaggccagcgtgcaccgaccgccgctgccggcctggcgccggacgacagcccccaggcccgtccgactagcgtcccggctccagcgcgctcctccggcggtgcgacggcgaccaccacctccggcagcacgttcccgactgcgctggcaccggcggctggcgctatcggcgggtcgggcggcaaccgcaccgccggtgcggagggcctgggcggctccagcaccgctggtggcggtggtccagatggctcgccgcagccgcagcggcctcctcctcaggagcagcagcagccgcaacgcccgcagcagcagaagcagcagcagcctgagccgagcccgccacaggagcagcctcgccagccggcagccgctgccgagcagcccgacgcggctcgccaaatcctcaacgagctgctcctgagccgcggtggcggcggtggtggcggtggtaatgccggcggtggtggcggtggcgacggtcccgccacgggcgatagcgacatggccaccgcagccgcgcccagcgctctgcacggggcgatcggtgcaggcgctggcgcatcgggtcccgcggcagctggccctgccgggagcggcggtggtggcggcagcggtggtggcggtggcggtggccagctgccgccctccggcgaggccgagacggcacgcggcaccctgcggtggcgcctggactcggctggcggtggcgagctcacgtgggacgagcgcgagcaggagtccctggtgccgctggccgccgacctgctgcaactgtggggccgtgtgctcctgctgctgctgcgccggagcctgtcgctgcgccaggctgcccccatgctggtgcgcctgcgccacacctcgctgcgcctggtgcggctggcggtgatgcaggctaccgcgcctgcgtgggtggagctgcacgtcctgccgctgcgcctgcggctggtgcagctgcaagtggtgggggcagcgcagtatggccctcccggctcggctgggggcgtggcgggcatggacgtggatggcggtgcgggttcggctgcgggtgcaggtggcggtggtggcggcctcggcctgcccacgtcgcagctcccgcctcagcccccacaaccaccgcagcccccgagcacagccctggcacccccgctccaactctcgcgtgagccttcgctgggccactacttcgaccacctgctgccccctgctgtgggtggtggcggtggtgccgctgcggcggccaccggcgcagtggctgccgccgctgcgccccctccccctccgctggccccaccggcgtggtccgggctgggcctgggcccaggtccgggcctgggcctgccgagcagcggcggtggtcctggcggcggtgccgccgctgcgccagagcctccgccacccccgcctcggccaccgcccccttacgactcgggcacgacgagcccgactgcgggcttcatcggcggtggtggcggcgcaggcggtcgggtgggtggtggcgtcgatggtggcggcggtggcgccgagggtggtggccgcagcggtggcggcgacggggcacgcgggggtgccgacggtggcgacgtggatggcagcggcggctcgggcggcagcggtggtggcggctccgtcgccagcggcggtgggggctcgggctccggcgcgtcccgtgcgctgagcgccagcatccgccaccccggtgggacgacgatcacactccagctgccccagggtcagcagcaacagcaacaacagcaacagcagcagcagcagcagcagcgtcagcagcagcagcagcagcaacagcaacagcagcagcaacagcagaatgccggtggcggtggcggcggcggcggcgacaagcccaccgacaccgccctggagcctccgccgccaccgccttgccaggccctgcgcaccggcgtggccgacaacatgcacgacttcgccccacctccgcctgcggccaccgccaccggcttcgcagctggcggtgtccctccccctcctcggacccctccacctagccccatgctcgtggacggtggcggtggcgcagcgggcggtggcggcagcggccctggcccagcgggtggtggtggcggggctggcggtcctggcggcggtgccggttacgtgcccagctccagcctgatgcagcagcagcagctgcaacaacctcagcagacccagcagcagccacagcccaaccccttctcgtccactgcgccgccaccgcctcccgctaccgcacccacaccggcaccgacaccgcccgctgctgccacctgggctgccgccgctgccccgtttcccgccacgcagtttccgcccgctgctccggccccttgcaccctgctctcgtcgagcgccgctgaccctttcatcgaggctcccagcggctcgaacacccctgtcggcattgggagcggtgggggtgccgccggtgagtgggccggtggcgtgcagcagcacgtgttccagcagcccctcgcggcaggcagcggcggtggcgtgggggctggcggcggtggcgagaagcgcgtggacgggggtggtggcggcggtggcggtgccttcgtgctgcccagcgcggctctcgctgcggccctctcgcctgcggaggtggccgccgtgcaggccgctgccgccctcatgcagcggggtgctgaccccgccgtggcgggcctgccacctcaggcagcggctatcgctctccaggcggctgcggccgaccagcaggccctggcggctgccgctgctgctgctgccggtgctgccggtggtgcggcctcggtgaatggtgccgctggcgcgcagctgcccctggcggctccctcgccggctgctccgtcgtcggccgcagcgtggggcgcagcaccggcagctatggcacctgccgcgggtccaccggcgtttctcccgagcgcggctgtgacgcctccggggagcaccgccgcaacgggcctgctgcccccagaggctgcggcagtgtaccccgctgctgccgaggcaccgcccccacccaccgcgttcagcgctccgcagcgctaaggatcc

SEQ ID NO: 244 - DNA, 3438 bp, artificial sequence
  codon optimized
cgtccgacggcacctccccctattagcaccactggcggtgggggtggcggttgctccgcagcagcggcgctccctgcggctgcgccccacgatgctgctcagacccacggcgcaggcgccgtggctggcggtggcggcgcaggggtgggcagcagcagctcgggcagcggcggcaacggtggtagctcgggcggctccagcagcagcgccggtggcggttcgggtggcggtggcgggggtggccccaccaccccaccagcgtccacgccacccccaagctcgcgcccgagcgtccctccgacccagcagcagcagcagcaacagcagcagcagcagcagcagcaacagcagcaacagcagcagcagcagcagccggcacccggcacctcggcaggccagcgtgcaccgaccgccgctgccggcctggcgccggacgacagcccccaggcccgtccgactagcgtcccggctccagcgcgctcctccggcggtgcgacggcgaccaccacctccggcagcacgttcccgactgcgctggcaccggcggctggcgctatcggcgggtcgggcggcaaccgcaccgccggtgcggagggcctgggcggctccagcaccgctggtggcggtggtccagatggctcgccgcagccgcagcggcctcctcctcaggagcagcagcagccgcaacgcccgcagcagcagaagcagcagcagcctgagccgagcccgccacaggagcagcctcgccagccggcagccgctgccgagcagcccgacgcggctcgccaaatcctcaacgagctgctcctgagccgcggtggcggcggtggtggcggtggtaatgccggcggtggtggcggtggcgacggtcccgccacgggcgatagcgacatggccaccgcagccgcgcccagcgctctgcacggggcgatcggtgcaggcgctggcgcatcgggtcccgcggcagctggccctgccgggagcggcggtggtggcggcagcggtggtggcggtggcggtggccagctgccgccctccggcgaggccgagacggcacgcggcaccctgcggtggcgcctggactcggctggcggtggcgagctcacgtgggacgagcgcgagcaggagtccctggtgccgctggccgccgacctgctgcaactgtggggccgtgtgctcctgctgctgctgcgccggagcctgtcgctgcgccaggctgcccccatgctggtgcgcctgcgccacacctcgctgcgcctggtgcggctggcggtgatgcaggctaccgcgcctgcgtgggtggagctgcacgtcctgccgctgcgcctgcggctggtgcagctgcaagtggtgggggcagcgcagtatggccctcccggctcggctgggggcgtggcgggcatggacgtggatggcggtgcgggttcggctgcgggtgcaggtggcggtggtggcggcctcggcctgcccacgtcgcagctcccgcctcagcccccacaaccaccgcagcccccgagcacagccctggcacccccgctccaactctcgcgtgagccttcgctgggccactacttcgaccacctgctgccccctgctgtgggtggtggcggtggtgccgctgcggcggccaccggcgcagtggctgccgccgctgcgccccctccccctccgctggccccaccggcgtggtccgggctgggcctgggcccaggtccgggcctgggcctgccgagcagcggcggtggtcctggcggcggtgccgccgctgcgccagagcctccgccacccccgcctcggccaccgcccccttacgactcgggcacgacgagcccgactgcgggcttcatcggcggtggtggcggcgcaggcggtcgggtgggtggtggcgtcgatggtggcggcggtggcgccgagggtggtggccgcagcggtggcggcgacggggcacgcgggggtgccgacggtggcgacgtggatggcagcggcggctcgggcggcagcggtggtggcggctccgtcgccagcggcggtgggggctcgggctccggcgcgtcccgtgcgctgagcgccagcatccgccaccccggtgggacgacgatcacactccagctgccccagggtcagcagcaacagcaacaacagcaacagcagcagcagcagcagcagcgtcagcagcagcagcagcagcaacagcaacagcagcagcaacagcagaatgccggtggcggtggcggcggcggcggcgacaagcccaccgacaccgccctggagcctccgccgccaccgccttgccaggccctgcgcaccggcgtggccgacaacatgcacgacttcgccccacctccgcctgcggccaccgccaccggcttcgcagctggcggtgtccctccccctcctcggacccctccacctagccccatgctcgtggacggtggcggtggcgcagcgggcggtggcggcagcggccctggcccagcgggtggtggtggcggggctggcggtcctggcggcggtgccggttacgtgcccagctccagcctgatgcagcagcagcagctgcaacaacctcagcagacccagcagcagccacagcccaaccccttctcgtccactgcgccgccaccgcctcccgctaccgcacccacaccggcaccgacaccgcccgctgctgccacctgggctgccgccgctgccccgtttcccgccacgcagtttccgcccgctgctccggccccttgcaccctgctctcgtcgagcgccgctgaccctttcatcgaggctcccagcggctcgaacacccctgtcggcattgggagcggtgggggtgccgccggtgagtgggccggtggcgtgcagcagcacgtgttccagcagcccctcgcggcaggcagcggcggtggcgtgggggctggcggcggtggcgagaagcgcgtggacgggggtggtggcggcggtggcggtgccttcgtgctgcccagcgcggctctcgctgcggccctctcgcctgcggaggtggccgccgtgcaggccgctgccgccctcatgcagcggggtgctgaccccgccgtggcgggcctgccacctcaggcagcggctatcgctctccaggcggctgcggccgaccagcaggccctggcggctgccgctgctgctgctgccggtgctgccggtggtgcggcctcggtgaatggtgccgctggcgcgcagctgcccctggcggctccctcgccggctgctccgtcgtcggccgcagcgtggggcgcagcaccggcagctatggcacctgccgcgggtccaccggcgtttctcccgagcgcggctgtgacgcctccggggagcaccgccgcaacgggcctgctgcccccagaggctgcggcagtgtaccccgctgctgccgaggcaccgcccccacccaccgcgttcagcgctccgcagcgc

SEQ ID NO: 245 - DNA, 3438 bp, Chlamydomonas reinhardtii
cggccgacggcaccgccgccaatctcaaccaccggcggcggcggcggcggctgctcggcggcggcggcactgcctgcggcggcgccgcatgacgccgcccagacgcacggcgctggagctgtagcaggcggcggcggcgcgggagtcggcagtagcagcagcggcagtggcggtaacggcggcagtagcggcggcagtagcagctcggctggcggtggcagtggcggtggcggcggcggcggtcccaccactccgccggcatcgacgccgccgccgtcgagtcgcccctcggtgccgccaacgcaacagcaacagcaacagcaacagcaacagcaacagcaacagcaacagcaacagcaacagcaacagcagccggcgccggggactagtgcagggcagcgcgcgccaacggcggcggcgggactcgcccctgatgactcaccgcaggcacgacccacatccgtacccgcacccgcccgcagcagcggcggtgctacagccaccaccacctctggctccaccttccccactgcccttgcccctgccgccggagccattggcggcagcggcggcaacagaaccgccggggcggaaggtctcggtggcagtagcacggccggcggcggcggccccgacggttcccctcagccgcagcgcccgccgccgcaagagcagcagcagccacagcggccgcagcaacagaagcaacagcagccggagccgtcgccgccacaggagcagccacgccagcccgccgccgccgccgagcaaccggacgcggcgcggcagatcctgaacgagctactgttgagccgcggcggcggcggcggcggcggcggcaatgcaggcggcggcggcggcggcgacggtccagccaccggcgacagtgacatggcaactgccgccgcgccgtctgctcttcatggcgctattggcgccggcgctggcgcttctgggccggcagcggcgggccctgcgggcagtggcggcggcggcggcagcggcggcggcggcggcggcggccagctgccgccaagcggcgaggcggagacggcgcgggggacactgcggtggcggctggactcggcgggtggcggcgagctgacgtgggatgagcgcgagcaggagtctttggtgcccctggcggcagacttgctacagctctggggccgggtgctactgctgctgctgcgacgcagcctcagcctgcggcaggcggcgccgatgctggtccggctgcgccacaccagcctgcgcctggtacggttggcggtgatgcaggcgaccgcgccggcatgggtggagctgcacgtgctgccgctgcggctgcgactggtccagctgcaggtggtgggggcggcgcagtatggccctcctggttcggcaggtggtgttgctggcatggacgtggatggtggcgccggatcagctgctggtgctggcggcggtggcggcggcctagggctgcccacctcccagctgccgccgcagccgccgcagccgccgcagccgccttccaccgccctggcgccgccgctgcagctgtcccgcgagccctccctgggccactacttcgaccacttgctgccaccagccgtaggcggcggcggcggggcggcggccgcagcaacaggggcagtggcagcggcggcggcgccgccaccgccgcccctggcgccgccggcctggtctggtctggggctgggaccggggccgggtctgggcctgccttctagcggcggcgggcccggcggcggtgcggctgctgctccggagccgccgccgccgccgccacggccaccgccgccgtacgacagcggtacgacctcgccaaccgctggctttattggtggcggcggtggtgctggaggtagagtaggaggcggcgttgatggcggcggtggcggtgcagagggaggcggtcgcagtgggggcggcgacggtgcgcgcggtggtgcagacggaggtgacgtggatgggagtggcggcagtggcggcagcggtggcggcggtagtgtcgccagtggcggcggcggtagcggcagtggcgccagtcgggcactgtccgcctccatccggcaccccggcggcaccaccatcacgctacagctgccgcagggacagcaacagcaacagcaacagcaacagcaacagcaacagcaacagcgacagcaacagcaacagcaacagcaacagcaacagcaacagcaacagaacgccggcggcggcggcggcggcggcggcgataaacccaccgacacggcgctcgagccgccgccgccaccgccatgccaggcgctgcggaccggtgtggcagacaacatgcacgacttcgcaccgccgccgccagccgctacggcgacgggctttgccgccggcggcgttccgccgccgccccggacgccgccgccctctcctatgctggtggacggcggcggcggtgctgctggcggcggcggcagtgggcccggccctgccggcggcggtggcggggctggaggtcccggtggcggtgctgggtacgtgccgtcttcgtcgctcatgcagcaacagcagctgcagcagccacaacaaacgcagcaacaaccgcaaccaaaccccttcagcagcacggcgccgccgccgccgcccgccaccgcgccaacccctgcacccacgccgccagcggctgcaacttgggcggcggcggcggccccgtttccggctacgcagttcccgcctgcggcgcccgcgccctgcacgttgctcagcagctctgcagctgacccctttatcgaggcgccatcgggtagcaacacgcctgtcggaattgggagcggcggcggggctgcgggagagtgggcaggtggtgtgcagcagcatgtgtttcagcagcccctggcggcggggagtggcgggggcgttggtgccggtggtggcggcgaaaagcgcgttgacggcggcggcggcggcggcggcggcgcgttcgtcctgccctcggcggcgttggcggcggcgctgtcgccggcggaggtggcggcagtgcaggcggcggcggcgctcatgcagcgcggcgccgacccggcggtggcgggcctgccgccacaggccgccgccatcgctctgcaggcggcggcggcggaccagcaagccctggcggcggccgccgccgccgccgccggcgctgccggcggtgctgctagtgtcaatggtgctgctggggcgcagttgcctctggcggccccgtctccagcggccccgtcttcagcggctgcttggggcgcagctcctgccgccatggcgcctgcggcgggcccgcctgcgttcctgccatctgctgcggtgacgccgcctggctcgacggccgcaacagggctgctgccgccggaggcggcggctgtgtacccggcggcggcagaggcgccgccgccgccgaccgctttcagtgcaccccagcgg

SEQ ID NO: 246 - protein, 1146 aa, Chlamydomonas reinhardtii
RPTAPPPISTTGGGGGGCSAAAALPAAAPHDAAQTHGAGAVAGGGGAGVGSSSSGSGGNGGSSGGSSSSAGGGSGGGGGGGPTTPPASTPPPSSRPSVPPTQQQQQQQQQQQQQQQQQQQQQQQPAPGTSAGQRAPTAAAGLAPDDSPQARPTSVPAPARSSGGATATTTSGSTFPTALAPAAGAIGGSGGNRTAGAEGLGGSSTAGGGGPDGSPQPQRPPPQEQQQPQRPQQQKQQQPEPSPPQEQPRQPAAAAEQPDAARQILNELLLSRGGGGGGGGNAGGGGGGDGPATGDSDMATAAAPSALHGAIGAGAGASGPAAAGPAGSGGGGGSGGGGGGGQLPPSGEAETARGTLRWRLDSAGGGELTWDEREQESLVPLAADLLQLWGRVLLLLLRRSLSLRQAAPMLVRLRHTSLRLVRLAVMQATAPAWVELHVLPLRLRLVQLQVVGAAQYGPPGSAGGVAGMDVDGGAGSAAGAGGGGGGLGLPTSQLPPQPPQPPQPPSTALAPPLQLSREPSLGHYFDHLLPPAVGGGGGAAAAATGAVAAAAAPPPPPLAPPAWSGLGLGPGPGLGLPSSGGGPGGGAAAAPEPPPPPPRPPPPYDSGTTSPTAGFIGGGGGAGGRVGGGVDGGGGGAEGGGRSGGGDGARGGADGGDVDGSGGSGGSGGGGSVASGGGGSGSGASRALSASIRHPGGTTITLQLPQGQQQQQQQQQQQQQQQRQQQQQQQQQQQQQQQNAGGGGGGGGDKPTDTALEPPPPPPCQALRTGVADNMHDFAPPPPAATATGFAAGGVPPPPRTPPPSPMLVDGGGGAAGGGGSGPGPAGGGGGAGGPGGGAGYVPSSSLMQQQQLQQPQQTQQQPQPNPFSSTAPPPPPATAPTPAPTPPAAATWAAAAAPFPATQFPPAAPAPCTLLSSSAADPFIEAPSGSNTPVGIGSGGGAAGEWAGGVQQHVFQQPLAAGSGGGVGAGGGGEKRVDGGGGGGGGAFVLPSAALAAALSPAEVAAVQAAAALMQRGADPAVAGLPPQAAAIALQAAAADQQALAAAAAAAAGAAGGAASVNGAAGAQLPLAAPSPAAPSSAAAWGAAPAAMAPAAGPPAFLPSAAVTPPGSTAATGLLPPEAAAVYPAAAEAPPPPTAFSAPQR

SEQ ID NO: 247 - DNA, 1620 bp, Chlamydomonas reinhardtii
atgtcaggagactttggctctgagcccctgggctcgtgctcagtggaaaccgttacggccttgctcggctatgggctcgagcaggacagcatcacggcgctttgccagcccgaggggggcgctggttgcaccagcacagataactgcatgttccagtatctgatgggcgcgaccgcggacgcgcagagcacggccagcgatgtgggagtcgggctcgacgtgagcttcctgttgttcagcggctatctggtattcgtgatgcagctcgggttcgctgtgctgtgcgctggaagcatccgctccaagaactgcatgaatatcctgctgaagaacatgctggacgcctgcgtgggcgctatcggcttctatgtcttcggatacgcgttcgcgtacggccgcaagtatggacagaatagcaacggcttcattggcaactggaactttgcgctctcgtacacaactcagacgtcaatgagcggcaccgagttcaccacgtttggatggcaccagttcttcttccagtggagcttctgcgccgccaccaccaccatcgtgtctggcgcggtggccgagcgctgcaccttcatggcctacatgatctacgcgttcttcctctcctccttcgtctaccccgtcatcgtgcactgggtgtgggacggccagggctggctcagtgccttcaacacattccaggacggctacgccctgatcctcaagaccggtgccattgactttgcgggctcgggcgtcgtgcacatgacgggtgggattgcggcactcatgggtgcctggattatgggcccccgtgtcggtcgcttcgctaatgacggcacggtgaatgagatgaggggccactcctccacgctggttgtgatgggcaccttcctgttgtggttcggctggttcggattcaaccccggcagcaacctggtggtcgcctcccaggctgcggccacagtcgtgtcgcgtgtcgcagtgaccaccgctctggcaggtggcgcgggtggaatctccatgctcttctacaagttcctgaccgtcaaggcttgggacgtggtggcgacgtgcaatggcatcctggctggcctcgtggccgtcacggctagctgctccgtcattgagccctgggcggccatcatcactggggccatcggggcaatcatcttttccattgctgattacgtgacgctttacaagctgaaggtggacgaccccgtgagcgcctttgccctgcacggcgccgtgggcgcatggggcgtgttgttccccggcttcctggcggctccccactatgtggtagaggtgtacggtgcctacggattcggaatggatgcacgggagggcaagaggttcggtctgttctacggcggccacggccaggtgctgctggtgcagctcatcgaggtgctggccatctttgggtggactggattcatgatgggcagcttcttcttcatcctcaacaaggctgggctgctgcgtgtgcccctgcaggaggagatggcgggtctggatgcggctaactacagcaagagcgttggcagcaaggacccctccgtccactgcaccgttggtgtggacaagctggaaggcggcgcgctgggggagggcaaggcctga

SEQ ID NO: 248 - protein, 539 aa, Chlamydomonas reinhardtii
MSGDFGSEPLGSCSVETVTALLGYGLEQDSITALCQPEGGAGCTSTDNCMFQYLMGATADAQSTASDVGVGLDVSFLLFSGYLVFVMQLGFAVLCAGSIRSKNCMNILLKNMLDACVGAIGFYVFGYAFAYGRKYGQNSNGFIGNWNFALSYTTQTSMSGTEFTTFGWHQFFFQWSFCAATTTIVSGAVAERCTFMAYMIYAFFLSSFVYPVIVHWVWDGQGWLSAFNTFQDGYALILKTGAIDFAGSGVVHMTGGIAALMGAWIMGPRVGRFANDGTVNEMRGHSSTLVVMGTFLLWFGWFGFNPGSNLVVASQAAATVVSRVAVTTALAGGAGGISMLFYKFLTVKAWDVVATCNGILAGLVAVTASCSVIEPWAAIITGAIGAIIFSIADYVTLYKLKVDDPVSAFALHGAVGAWGVLFPGFLAAPHYVVEVYGAYGFGMDAREGKRFGLFYGGHGQVLLVQLIEVLAIFGWTGFMMGSFFFILNKAGLLRVPLQEEMAGLDAANYSKSVGSKDPSVHCTVGVDKLEGGALGEGKA

SEQ ID NO: 249 - DNA, 1635 bp, artificial sequence
  codon optimized
catatgctcgagtcgggtgattttggtagcgagcctctgggttcgtgtagcgtggagacggtgaccgccctgctgggctacggtctggagcaggacagcatcaccgctctgtgccagcctgagggcggcgccggctgcactagcaccgacaactgcatgttccagtacctgatgggcgcgacggcggatgcacagagcaccgcctccgacgtgggcgtggggctggacgtgagcttcctgctcttcagcggctacctggtctttgtgatgcagctggggttcgccgtgctctgcgccggcagcatccggtccaagaactgcatgaacatcctgctcaagaacatgctggacgcctgtgtgggcgccattggtttctacgtcttcgggtacgccttcgcgtacggccgcaagtacggccagaactccaacggcttcatcggcaactggaacttcgccctgtcgtacacgacccagacctcgatgtcgggcaccgagttcaccaccttcggctggcaccagtttttcttccagtggtcgttctgcgcggctaccacgaccatcgtgagcggcgcagtcgctgagcggtgcacctttatggcctacatgatctacgccttctttctgagctcgttcgtgtacccggtgattgtgcactgggtgtgggacggccagggctggctgagcgcctttaacaccttccaagacggctacgcgctcatcctgaagaccggggccatcgactttgcgggtagcggcgtggtgcacatgaccggcggcatcgcagcgctcatgggcgcctggattatgggtccacgtgtgggtcgctttgcgaacgacggcacagtgaacgagatgcgcggtcactcctcgacgctggtggtgatggggactttcctcctgtggttcgggtggttcggcttcaaccccggcagcaacctggtggtggcttcccaggccgcagccactgtggtgagccgcgtcgccgtcacaacggccctggctgggggcgctggtggcatctcgatgctcttttacaagttcctgacggtgaaggcgtgggacgtggtcgccacttgcaacggcatcctcgccggtctggtggctgtgacagcctcctgtagcgtgatcgagccctgggcggcaatcatcaccggcgcgatcggggcgatcatctttagcatcgctgactacgtgaccctgtataagctgaaggtggacgacccggtgtcggcgttcgccctgcacggcgcggtgggcgcgtggggcgtcctgttccccggcttcctggctgcgccccactacgtggtggaggtgtacggcgcctacggcttcggcatggacgcgcgcgaggggaagcggttcggtctgttctacggcggtcatggccaggtgctcctggtgcaactgatcgaggtgctggccattttcggctggaccggcttcatgatgggcagcttcttcttcatcctcaacaaggccgggctgctgcgcgtccccctccaagaggagatggctggcctggacgcggcgaactacagcaagtccgtgggctcgaaggacccctcggtccactgcaccgtgggggtggacaagctggagggtggcgctctgggcgagggcaaggcgtaaggatcc

SEQ ID NO: 250 - DNA, 1614 bp, artificial sequence
  codon optimized
tcgggtgattttggtagcgagcctctgggttcgtgtagcgtggagacggtgaccgccctgctgggctacggtctggagcaggacagcatcaccgctctgtgccagcctgagggcggcgccggctgcactagcaccgacaactgcatgttccagtacctgatgggcgcgacggcggatgcacagagcaccgcctccgacgtgggcgtggggctggacgtgagcttcctgctcttcagcggctacctggtctttgtgatgcagctggggttcgccgtgctctgcgccggcagcatccggtccaagaactgcatgaacatcctgctcaagaacatgctggacgcctgtgtgggcgccattggtttctacgtcttcgggtacgccttcgcgtacggccgcaagtacggccagaactccaacggcttcatcggcaactggaacttcgccctgtcgtacacgacccagacctcgatgtcgggcaccgagttcaccaccttcggctggcaccagtttttcttccagtggtcgttctgcgcggctaccacgaccatcgtgagcggcgcagtcgctgagcggtgcacctttatggcctacatgatctacgccttctttctgagctcgttcgtgtacccggtgattgtgcactgggtgtgggacggccagggctggctgagcgcctttaacaccttccaagacggctacgcgctcatcctgaagaccggggccatcgactttgcgggtagcggcgtggtgcacatgaccggcggcatcgcagcgctcatgggcgcctggattatgggtccacgtgtgggtcgctttgcgaacgacggcacagtgaacgagatgcgcggtcactcctcgacgctggtggtgatggggactttcctcctgtggttcgggtggttcggcttcaaccccggcagcaacctggtggtggcttcccaggccgcagccactgtggtgagccgcgtcgccgtcacaacggccctggctgggggcgctggtggcatctcgatgctcttttacaagttcctgacggtgaaggcgtgggacgtggtcgccacttgcaacggcatcctcgccggtctggtggctgtgacagcctcctgtagcgtgatcgagccctgggcggcaatcatcaccggcgcgatcggggcgatcatctttagcatcgctgactacgtgaccctgtataagctgaaggtggacgacccggtgtcggcgttcgccctgcacggcgcggtgggcgcgtggggcgtcctgttccccggcttcctggctgcgccccactacgtggtggaggtgtacggcgcctacggcttcggcatggacgcgcgcgaggggaagcggttcggtctgttctacggcggtcatggccaggtgctcctggtgcaactgatcgaggtgctggccattttcggctggaccggcttcatgatgggcagcttcttcttcatcctcaacaaggccgggctgctgcgcgtccccctccaagaggagatggctggcctggacgcggcgaactacagcaagtccgtgggctcgaaggacccctcggtccactgcaccgtgggggtggacaagctggagggtggcgctctgggcgagggcaaggcg

SEQ ID NO: 251 - DNA, 1614 bp, Chlamydomonas reinhardtii
tcaggagactttggctctgagcccctgggctcgtgctcagtggaaaccgttacggccttgctcggctatgggctcgagcaggacagcatcacggcgctttgccagcccgaggggggcgctggttgcaccagcacagataactgcatgttccagtatctgatgggcgcgaccgcggacgcgcagagcacggccagcgatgtgggagtcgggctcgacgtgagcttcctgttgttcagcggctatctggtattcgtgatgcagctcgggttcgctgtgctgtgcgctggaagcatccgctccaagaactgcatgaatatcctgctgaagaacatgctggacgcctgcgtgggcgctatcggcttctatgtcttcggatacgcgttcgcgtacggccgcaagtatggacagaatagcaacggcttcattggcaactggaactttgcgctctcgtacacaactcagacgtcaatgagcggcaccgagttcaccacgtttggatggcaccagttcttcttccagtggagcttctgcgccgccaccaccaccatcgtgtctggcgcggtggccgagcgctgcaccttcatggcctacatgatctacgcgttcttcctctcctccttcgtctaccccgtcatcgtgcactgggtgtgggacggccagggctggctcagtgccttcaacacattccaggacggctacgccctgatcctcaagaccggtgccattgactttgcgggctcgggcgtcgtgcacatgacgggtgggattgcggcactcatgggtgcctggattatgggcccccgtgtcggtcgcttcgctaatgacggcacggtgaatgagatgaggggccactcctccacgctggttgtgatgggcaccttcctgttgtggttcggctggttcggattcaaccccggcagcaacctggtggtcgcctcccaggctgcggccacagtcgtgtcgcgtgtcgcagtgaccaccgctctggcaggtggcgcgggtggaatctccatgctcttctacaagttcctgaccgtcaaggcttgggacgtggtggcgacgtgcaatggcatcctggctggcctcgtggccgtcacggctagctgctccgtcattgagccctgggcggccatcatcactggggccatcggggcaatcatcttttccattgctgattacgtgacgctttacaagctgaaggtggacgaccccgtgagcgcctttgccctgcacggcgccgtgggcgcatggggcgtgttgttccccggcttcctggcggctccccactatgtggtagaggtgtacggtgcctacggattcggaatggatgcacgggagggcaagaggttcggtctgttctacggcggccacggccaggtgctgctggtgcagctcatcgaggtgctggccatctttgggtggactggattcatgatgggcagcttcttcttcatcctcaacaaggctgggctgctgcgtgtgcccctgcaggaggagatggcgggtctggatgcggctaactacagcaagagcgttggcagcaaggacccctccgtccactgcaccgttggtgtggacaagctggaaggcggcgcgctgggggagggcaaggcc

SEQ ID NO: 252 - protein, 538 aa, Chlamydomonas reinhardtii
SGDFGSEPLGSCSVETVTALLGYGLEQDSITALCQPEGGAGCTSTDNCMFQYLMGATADAQSTASDVGVGLDVSFLLFSGYLVFVMQLGFAVLCAGSIRSKNCMNILLKNMLDACVGAIGFYVFGYAFAYGRKYGQNSNGFIGNWNFALSYTTQTSMSGTEFTTFGWHQFFFQWSFCAATTTIVSGAVAERCTFMAYMIYAFFLSSFVYPVIVHWVWDGQGWLSAFNTFQDGYALILKTGAIDFAGSGVVHMTGGIAALMGAWIMGPRVGRFANDGTVNEMRGHSSTLVVMGTFLLWFGWFGFNPGSNLVVASQAAATVVSRVAVTTALAGGAGGISMLFYKFLTVKAWDVVATCNGILAGLVAVTASCSVIEPWAAIITGAIGAIIFSIADYVTLYKLKVDDPVSAFALHGAVGAWGVLFPGFLAAPHYVVEVYGAYGFGMDAREGKRFGLFYGGHGQVLLVQLIEVLAIFGWTGFMMGSFFFILNKAGLLRVPLQEEMAGLDAANYSKSVGSKDPSVHCTVGVDKLEGGALGEGKA

SEQ ID NO: 253 - DNA, 1770 bp, Chlamydomonas reinhardtii
atgcagtcgcgccagtgcttgaaccgcaaggccagcggcgcgcggccctgcgctaactcgcgcagcctcacagctcgcgtactcgctacggccgcgcctgtcgcgccgtccgccacacccgcctccgcccccctgcccctccccgatggcgttggcgagcacagcggcctgaagcacctgcccgaggccgcccgcactcgtgcgctcgacaagaaggccaacaagtttgagaaggttaaggtcgagaagtgcggctcgcgcgcctggaacgacgtgtttgagctgtcttccctgctgaaggagggcaagaccaagtgggaggaccttaacctcgatgatgtcgacatccgtctcaagtgggccggcctgttccaccgcggcaagcgcacccccggcaagttcatgatgcgtctcaaggtgcccaacggcgagctcaccgccgcgcagctgcgcttcctggcctcctccatcgcgccctacggcgctgacggctgcgccgacatcaccacccgcgccaacatccagctgcgcggcgtcaccatggaggactcggagacggtcatcaaggggctgtgggatgtgggcctgacgtccttccagtcgggcatggactccgtgcgcaacctcaccggcaaccccatcgccggagtcgacccacacgagctggtggacacgcggccgctgctgcgcgacatggaggcgatgctgttcaacaacggcaagggccgcgaggagtttgccaacctgccgcgcaagctgaacatctgcatctcctccacccgcgacgacttcccgcacacccacatcaacgacgttggctacgaggccgtggccaagcccaacggcgaggtggtgtacaatgtggtggtgggcggctacttctccatcaagcgcaacatcatgtccatcccgctgggctgctccatcacccaggaccagctgatgcccttcactgaggccctgctgcgcgtgttccgggatcacggcccgcgcggcgaccggcagcagacgcggctgatgtggctggtggaggcggtgggcgtggacaagttccgccagctgctgtcggagtacatgggcggcgccaccttcggcgagcccgtgcacgttcaccacgaccagccctgggagcggcgcaacctgctgggcgtgcaccggcagaggcaggccggcctgaactgggtcggcgcctgcgtgcccgcgggccgcctgcacgccgccgactttgaggagatcgcggctgtggctgagaagtacggcgacggcacggtgcgcatcacgtgcgaggagaacgtgatcttcaccaacgtgcccgacgccaagctggaggcgatgaaggcggagccgctgttccagcgcttccccatcttccccggcgtgctgctgtcgggcatggtgtcctgcaccggcaaccagttctgcggcttcggtctggctgagaccaaggcgaaggccgtgaaggtggtggaggcgctggacgcgcagctggagctgagccggcccgtgcgcatccacttcaccggctgccccaactcatgcggccaggcgcaggtgggcgacatcgggctgatgggcgcgcccgccaagcacgagggcaaggccgtggagggctacaagatcttcctgggcggcaagatcggcgagaaccccgcgctcgccaccgagttcgcgcagggtgtgccggccattgagagcgtgctggtgcctcggctaaaggagattctgatctccgagttcggtgccaaggagcgcgccaccgccaccgcctaa

SEQ ID NO: 254 - protein, 589 aa, Chlamydomonas reinhardtii
MQSRQCLNRKASGARPCANSRSLTARVLATAAPVAPSATPASAPLPLPDGVGEHSGLKHLPEAARTRALDKKANKFEKVKVEKCGSRAWNDVFELSSLLKEGKTKWEDLNLDDVDIRLKWAGLFHRGKRTPGKFMMRLKVPNGELTAAQLRFLASSIAPYGADGCADITTRANIQLRGVTMEDSETVIKGLWDVGLTSFQSGMDSVRNLTGNPIAGVDPHELVDTRPLLRDMEAMLFNNGKGREEFANLPRKLNICISSTRDDFPHTHINDVGYEAVAKPNGEVVYNVVVGGYFSIKRNIMSIPLGCSITQDQLMPFTEALLRVFRDHGPRGDRQQTRLMWLVEAVGVDKFRQLLSEYMGGATFGEPVHVHHDQPWERRNLLGVHRQRQAGLNWVGACVPAGRLHAADFEEIAAVAEKYGDGTVRITCEENVIFTNVPDAKLEAMKAEPLFQRFPIFPGVLLSGMVSCTGNQFCGFGLAETKAKAVKVVEALDAQLELSRPVRIHFTGCPNSCGQAQVGDIGLMGAPAKHEGKAVEGYKIFLGGKIGENPALATEFAQGVPAIESVLVPRLKEILISEFGAKERATATA

SEQ ID NO: 255 - DNA, 1791 bp, artificial sequence
  codon optimized
catatgctcgagcaatcccggcagtgtctcaatcgcaaggcttcgggtgcacggccatgcgcaaacagccgctcgctgaccgcccgtgtgctggcaacggctgcccccgtggcgccttccgccacgccggcgagcgccccactgccactgcccgacggcgtgggcgagcactccgggctgaagcatctgcccgaggcggcccgcacacgcgccctggacaagaaggccaacaagttcgagaaggtgaaggtggagaagtgcggctcgcgcgcctggaacgatgtctttgagctgtcctccctgctcaaggagggcaagaccaagtgggaggacctgaacctggacgacgtggacattcgtctgaagtgggctggcctctttcaccgcggcaagcgcacgccagggaagttcatgatgcgtctgaaggtgcccaatggcgagctgaccgcggcacagctgcggttcctggcctcgtcgatcgctccgtacggcgccgacggctgcgccgacatcacaacacgggccaacattcagctgcgcggcgtgacgatggaggactcggagacggtcatcaagggcctgtgggacgtgggtctgacctccttccagagcggcatggactccgtgcgcaacctcactggcaaccccatcgcgggggtggacccccatgagctggtggacactcggcccctcctgcgtgacatggaggccatgctgttcaacaacggcaagggtcgtgaggagttcgccaacctgccgcgcaagctgaacatctgtatcagctccacgcgggatgacttcccccacacccatattaacgacgtgggctacgaggccgtcgcgaagcccaacggcgaggtggtgtacaacgtcgtggtcggcggctacttctccatcaagcgtaacattatgagcattccgctgggctgctcgattacccaggaccagctgatgccctttaccgaggcgctgctccgcgtcttccgcgaccacggccctcggggcgaccgccaacaaacccgcctgatgtggctggtggaggccgtgggcgtggacaagttccggcagctcctcagcgagtacatgggcggcgccacgttcggcgagcccgtgcacgtccaccacgaccagccctgggagcggcgcaacctgctgggcgtccaccgccagcggcaggcaggtctgaactgggtgggtgcttgtgtgcccgctggccgcctgcacgccgctgacttcgaggagatcgccgcggtggccgagaagtatggcgacggcactgtgcgcatcacctgcgaggagaacgtgatcttcaccaacgtccccgatgctaagctggaggcgatgaaggccgagcctctgttccaacggttccccatctttccgggggtgctgctgtcgggcatggtgtcctgcacgggtaaccagttctgcggcttcggtctggcggagacgaaggcgaaggctgtcaaggtggtcgaggctctggacgcccaactggagctgagccgcccggtgcgcatccacttcacgggctgccccaactcctgcggccaggcgcaggtgggcgacatcggcctgatgggcgcacctgccaagcacgagggcaaggccgtggagggctacaagattttcctgggcggcaagatcggcgagaacccggccctcgcgacggagttcgcccagggcgtgcctgccatcgagagcgtgctcgtgccgcgcctgaaggagattctgatttcggagtttggcgcgaaggagcgcgccacggctaccgctaccggttagggatcc

SEQ ID NO: 256 - DNA, 1764 bp, artificial sequence
  codon optimized
caatcccggcagtgtctcaatcgcaaggcttcgggtgcacggccatgcgcaaacagccgctcgctgaccgcccgtgtgctggcaacggctgcccccgtggcgccttccgccacgccggcgagcgccccactgccactgcccgacggcgtgggcgagcactccgggctgaagcatctgcccgaggcggcccgcacacgcgccctggacaagaaggccaacaagttcgagaaggtgaaggtggagaagtgcggctcgcgcgcctggaacgatgtctttgagctgtcctccctgctcaaggagggcaagaccaagtgggaggacctgaacctggacgacgtggacattcgtctgaagtgggctggcctctttcaccgcggcaagcgcacgccagggaagttcatgatgcgtctgaaggtgcccaatggcgagctgaccgcggcacagctgcggttcctggcctcgtcgatcgctccgtacggcgccgacggctgcgccgacatcacaacacgggccaacattcagctgcgcggcgtgacgatggaggactcggagacggtcatcaagggcctgtgggacgtgggtctgacctccttccagagcggcatggactccgtgcgcaacctcactggcaaccccatcgcgggggtggacccccatgagctggtggacactcggcccctcctgcgtgacatggaggccatgctgttcaacaacggcaagggtcgtgaggagttcgccaacctgccgcgcaagctgaacatctgtatcagctccacgcgggatgacttcccccacacccatattaacgacgtgggctacgaggccgtcgcgaagcccaacggcgaggtggtgtacaacgtcgtggtcggcggctacttctccatcaagcgtaacattatgagcattccgctgggctgctcgattacccaggaccagctgatgccctttaccgaggcgctgctccgcgtcttccgcgaccacggccctcggggcgaccgccaacaaacccgcctgatgtggctggtggaggccgtgggcgtggacaagttccggcagctcctcagcgagtacatgggcggcgccacgttcggcgagcccgtgcacgtccaccacgaccagccctgggagcggcgcaacctgctgggcgtccaccgccagcggcaggcaggtctgaactgggtgggtgcttgtgtgcccgctggccgcctgcacgccgctgacttcgaggagatcgccgcggtggccgagaagtatggcgacggcactgtgcgcatcacctgcgaggagaacgtgatcttcaccaacgtccccgatgctaagctggaggcgatgaaggccgagcctctgttccaacggttccccatctttccgggggtgctgctgtcgggcatggtgtcctgcacgggtaaccagttctgcggcttcggtctggcggagacgaaggcgaaggctgtcaaggtggtcgaggctctggacgcccaactggagctgagccgcccggtgcgcatccacttcacgggctgccccaactcctgcggccaggcgcaggtgggcgacatcggcctgatgggcgcacctgccaagcacgagggcaaggccgtggagggctacaagattttcctgggcggcaagatcggcgagaacccggccctcgcgacggagttcgcccagggcgtgcctgccatcgagagcgtgctcgtgccgcgcctgaaggagattctgatttcggagtttggcgcgaaggagcgcgccacggctaccgct

SEQ ID NO: 257 - DNA, 1764 bp, Chlamydomonas reinhardtii
cagtcgcgccagtgcttgaaccgcaaggccagcggcgcgcggccctgcgctaactcgcgcagcctcacagctcgcgtactcgctacggccgcgcctgtcgcgccgtccgccacacccgcctccgcccccctgcccctccccgatggcgttggcgagcacagcggcctgaagcacctgcccgaggccgcccgcactcgtgcgctcgacaagaaggccaacaagtttgagaaggttaaggtcgagaagtgcggctcgcgcgcctggaacgacgtgtttgagctgtcttccctgctgaaggagggcaagaccaagtgggaggaccttaacctcgatgatgtcgacatccgtctcaagtgggccggcctgttccaccgcggcaagcgcacccccggcaagttcatgatgcgtctcaaggtgcccaacggcgagctcaccgccgcgcagctgcgcttcctggcctcctccatcgcgccctacggcgctgacggctgcgccgacatcaccacccgcgccaacatccagctgcgcggcgtcaccatggaggactcggagacggtcatcaaggggctgtgggatgtgggcctgacgtccttccagtcgggcatggactccgtgcgcaacctcaccggcaaccccatcgccggagtcgacccacacgagctggtggacacgcggccgctgctgcgcgacatggaggcgatgctgttcaacaacggcaagggccgcgaggagtttgccaacctgccgcgcaagctgaacatctgcatctcctccacccgcgacgacttcccgcacacccacatcaacgacgttggctacgaggccgtggccaagcccaacggcgaggtggtgtacaatgtggtggtgggcggctacttctccatcaagcgcaacatcatgtccatcccgctgggctgctccatcacccaggaccagctgatgcccttcactgaggccctgctgcgcgtgttccgggatcacggcccgcgcggcgaccggcagcagacgcggctgatgtggctggtggaggcggtgggcgtggacaagttccgccagctgctgtcggagtacatgggcggcgccaccttcggcgagcccgtgcacgttcaccacgaccagccctgggagcggcgcaacctgctgggcgtgcaccggcagaggcaggccggcctgaactgggtcggcgcctgcgtgcccgcgggccgcctgcacgccgccgactttgaggagatcgcggctgtggctgagaagtacggcgacggcacggtgcgcatcacgtgcgaggagaacgtgatcttcaccaacgtgcccgacgccaagctggaggcgatgaaggcggagccgctgttccagcgcttccccatcttccccggcgtgctgctgtcgggcatggtgtcctgcaccggcaaccagttctgcggcttcggtctggctgagaccaaggcgaaggccgtgaaggtggtggaggcgctggacgcgcagctggagctgagccggcccgtgcgcatccacttcaccggctgccccaactcatgcggccaggcgcaggtgggcgacatcgggctgatgggcgcgcccgccaagcacgagggcaaggccgtggagggctacaagatcttcctgggcggcaagatcggcgagaaccccgcgctcgccaccgagttcgcgcagggtgtgccggccattgagagcgtgctggtgcctcggctaaaggagattctgatctccgagttcggtgccaaggagcgcgccaccgccaccgcc

SEQ ID NO: 258 - protein, 588 aa, Chlamydomonas reinhardtii
QSRQCLNRKASGARPCANSRSLTARVLATAAPVAPSATPASAPLPLPDGVGEHSGLKHLPEAARTRALDKKANKFEKVKVEKCGSRAWNDVFELSSLLKEGKTKWEDLNLDDVDIRLKWAGLFHRGKRTPGKFMMRLKVPNGELTAAQLRFLASSIAPYGADGCADITTRANIQLRGVTMEDSETVIKGLWDVGLTSFQSGMDSVRNLTGNPIAGVDPHELVDTRPLLRDMEAMLFNNGKGREEFANLPRKLNICISSTRDDFPHTHINDVGYEAVAKPNGEVVYNVVVGGYFSIKRNIMSIPLGCSITQDQLMPFTEALLRVFRDHGPRGDRQQTRLMWLVEAVGVDKFRQLLSEYMGGATFGEPVHVHHDQPWERRNLLGVHRQRQAGLNWVGACVPAGRLHAADFEEIAAVAEKYGDGTVRITCEENVIFTNVPDAKLEAMKAEPLFQRFPIFPGVLLSGMVSCTGNQFCGFGLAETKAKAVKVVEALDAQLELSRPVRIHFTGCPNSCGQAQVGDIGLMGAPAKHEGKAVEGYKIFLGGKIGENPALATEFAQGVPAIESVLVPRLKEILISEFGAKERATATA

SEQ ID NO: 259 - DNA, 756 bp, Chlamydomonas reinhardtii
atgggcggggccggccatggtacaagcagcacggagggcggcggcggcggcggcggcggcagcagcagcagcagcaacagtgggggtggcggcagtagcttcgacgctgaggtgctcacgctcatcatgcatgcggatgacatcttctgcagcgctgacggcgagcctattcgggtgctgcttcagcagcaagggccgcctgaagtgctcatcaatgaagctgtcgcctgcttcggcacgcgcccgctgctcaaggtgctgaccatcctggtcaacggcctgttggagcggttgcgtggcggcgccagtgccgaccgcactgcagcggcggcagcagtgggcagtggggcagcggcgaagccggcgcccgccggtataacgcctggggtggagccggcggctggtagtgatgctgcggccgtgccagctgccaggactggccgcgtgagcagtgtttattggggtgctgagatgagccttgacaagatccgcgaggtggccagcagccaggagggtcttgagtggcttgtgcagggagggggcggctcgctgcgcccagacatgccgctcattgtagtcatctcggatgacgacgcgacctggcagcagtgccgcagctttgtggagatgaaggcgccagcactgatgctggagggcagaacgccgcgctcgctggtggacctgtggctgagcggcgacacacatgttcgcgccatattggcacagtactcgctcgtacgctcgttcatgccttaa

SEQ ID NO: 260 - protein, 251 aa, Chlamydomonas reinhardtii
MGGAGHGTSSTEGGGGGGGGSSSSSNSGGGGSSFDAEVLTLIMHADDIFCSADGEPIRVLLQQQGPPEVLINEAVACFGTRPLLKVLTILVNGLLERLRGGASADRTAAAAAVGSGAAAKPAPAGITPGVEPAAGSDAAAVPAARTGRVSSVYWGAEMSLDKIREVASSQEGLEWLVQGGGGSLRPDMPLIVVISDDDATWQQCRSFVEMKAPALMLEGRTPRSLVDLWLSGDTHVRAILAQYSLVRSFMP

SEQ ID NO: 261 - DNA, 771 bp, artificial sequence
  codon optimized
catatgctcgagggtggcgcaggtcatggtacaagcagcacagagggtggtggtggtggcggtggcggctcctcgtccagctccaactccggtggcggcggttcctcgttcgacgctgaggtcctgacgctgatcatgcacgctgacgacatcttctgctccgccgacggggagccgatccgcgtcctcctgcaacaacaagggccgcctgaggtgctgattaacgaggcggtggcctgcttcggcacccgtcccctgctgaaggtgctgacgatcctggtgaacggcctgctggagcggctgcggggcggggcgagcgcagaccgcaccgctgccgctgccgcggtgggcagcggtgcggcggctaagcccgcgcctgcgggtatcactccgggcgtggagccggctgccggctcggacgccgctgctgtgcctgccgcacgcacgggtcgcgtctcctccgtctactggggcgcagagatgtcgctggacaagatccgcgaggtggcgagctcccaggagggcctggagtggctggtccagggcggtgggggcagcctccgcccggacatgccactcattgtggtgatttcggacgacgacgcgacgtggcagcagtgccgttcgttcgtcgagatgaaggcacccgcgctgatgctggagggccgtacgccgcgctccctggtggacctgtggctcagcggcgacacccacgtgcgcgcaatcctggcccagtactcgctggtgcgcagcttcatgccataaggatcc

SEQ ID NO: 262 - DNA, 750 bp, artificial sequence
  codon optimized
ggtggcgcaggtcatggtacaagcagcacagagggtggtggtggtggcggtggcggctcctcgtccagctccaactccggtggcggcggttcctcgttcgacgctgaggtcctgacgctgatcatgcacgctgacgacatcttctgctccgccgacggggagccgatccgcgtcctcctgcaacaacaagggccgcctgaggtgctgattaacgaggcggtggcctgcttcggcacccgtcccctgctgaaggtgctgacgatcctggtgaacggcctgctggagcggctgcggggcggggcgagcgcagaccgcaccgctgccgctgccgcggtgggcagcggtgcggcggctaagcccgcgcctgcgggtatcactccgggcgtggagccggctgccggctcggacgccgctgctgtgcctgccgcacgcacgggtcgcgtctcctccgtctactggggcgcagagatgtcgctggacaagatccgcgaggtggcgagctcccaggagggcctggagtggctggtccagggcggtgggggcagcctccgcccggacatgccactcattgtggtgatttcggacgacgacgcgacgtggcagcagtgccgttcgttcgtcgagatgaaggcacccgcgctgatgctggagggccgtacgccgcgctccctggtggacctgtggctcagcggcgacacccacgtgcgcgcaatcctggcccagtactcgctggtgcgcagcttcatgcca

SEQ ID NO: 263 - DNA, 750 bp, Chlamydomonas reinhardtii
ggcggggccggccatggtacaagcagcacggagggcggcggcggcggcggcggcggcagcagcagcagcagcaacagtgggggtggcggcagtagcttcgacgctgaggtgctcacgctcatcatgcatgcggatgacatcttctgcagcgctgacggcgagcctattcgggtgctgcttcagcagcaagggccgcctgaagtgctcatcaatgaagctgtcgcctgcttcggcacgcgcccgctgctcaaggtgctgaccatcctggtcaacggcctgttggagcggttgcgtggcggcgccagtgccgaccgcactgcagcggcggcagcagtgggcagtggggcagcggcgaagccggcgcccgccggtataacgcctggggtggagccggcggctggtagtgatgctgcggccgtgccagctgccaggactggccgcgtgagcagtgtttattggggtgctgagatgagccttgacaagatccgcgaggtggccagcagccaggagggtcttgagtggcttgtgcagggagggggcggctcgctgcgcccagacatgccgctcattgtagtcatctcggatgacgacgcgacctggcagcagtgccgcagctttgtggagatgaaggcgccagcactgatgctggagggcagaacgccgcgctcgctggtggacctgtggctgagcggcgacacacatgttcgcgccatattggcacagtactcgctcgtacgctcgttcatgcct

SEQ ID NO: 264 - protein, 250 aa, Chlamydomonas reinhardtii
GGAGHGTSSTEGGGGGGGGSSSSSNSGGGGSSFDAEVLTLIMHADDIFCSADGEPIRVLLQQQGPPEVLINEAVACFGTRPLLKVLTILVNGLLERLRGGASADRTAAAAAVGSGAAAKPAPAGITPGVEPAAGSDAAAVPAARTGRVSSVYWGAEMSLDKIREVASSQEGLEWLVQGGGGSLRPDMPLIVVISDDDATWQQCRSFVEMKAPALMLEGRTPRSLVDLWLSGDTHVRAILAQYSLVRSFMP

SEQ ID NO: 265 - DNA, 795 bp, Chlamydomonas reinhardtii
atgatacctgcacaaaacgaaaagccccacaagggcgtgttgctgcaggcgggctggctgcatttgtcaagtccggaagaacggaaagccgctgaggttcaggccgaggcgacccggctcatgcctccgccgcgcctgccgggcgccgcctccgccccagctgtggaaatggttcccaccgctgacggcccccccaagtgccccgtgccgtcatgggcaggagagccgccggcggggtcgcggctgcttgtgtacaaggaggggacagtcatccaggacattgcactgggcaaggtggtgactgtgttcggccgtgtgccggagctggcggacgtggtgctggaccacccctccatcagccgccagcacgccaccgccgcctggcacccgggccgcgccgcctggctgctcaccgacctgggctccacacacggcacctgggtgggcgacagccgcctgggcaagaacgagcccgccgagctggtgcctggtgtggaggtccgcttcgccgcctccacccgcagatacaagctggcggcgccgctgggcggcaaggcggcggcggcggcggggaactgtgcccgtggggatgcgggtgacgcagccgccccgggtacggcgccagggtcggggctgatgctgccaccgccgccgaagcggccgcgagtatctttcgccgacgacgactcggccgctgcgggcggcggtggcggcggtgcgccgcggtctgcgctggagactgtcattggcttcacggacggcaaggactttgtgcgagcttcaaagctcatctaa

SEQ ID NO: 266 - protein, 264 aa, Chlamydomonas reinhardtii
MIPAQNEKPHKGVLLQAGWLHLSSPEERKAAEVQAEATRLMPPPRLPGAASAPAVEMVPTADGPPKCPVPSWAGEPPAGSRLLVYKEGTVIQDIALGKVVTVFGRVPELADVVLDHPSISRQHATAAWHPGRAAWLLTDLGSTHGTWVGDSRLGKNEPAELVPGVEVRFAASTRRYKLAAPLGGKAAAAAGNCARGDAGDAAAPGTAPGSGLMLPPPPKRPRVSFADDDSAAAGGGGGGAPRSALETVIGFTDGKDFVRASKLI

SEQ ID NO: 267 - DNA, 810 bp, artificial sequence
  codon optimized
catatgctcgagattcctgctcaaaatgagaagccacacaagggggtgctgctgcaagcagggtggctgcacctgtcctcgcccgaggagcggaaggctgctgaggtgcaagcggaggcgacccgcctgatgccgcctccacgtctgcccggtgctgccagcgcccctgctgtggagatggtcccgactgcggacggtccaccgaagtgccccgtgccgtcctgggctggcgagccgccagcaggctcccgcctgctggtgtacaaggagggcacggtgatccaggacatcgccctgggcaaggtggtgactgtgttcggccgcgtcccagagctggcggacgtggtgctggaccacccctcgatttcgcgccaacacgccaccgcggcctggcaccctggccgcgctgcctggctcctgaccgacctcggctccacacacggcacatgggtgggcgactcgcggctgggcaagaacgagccggccgagctggtgcccggcgtcgaggtccgctttgcggccagcacgcgccgctacaagctcgccgctcccctgggcggcaaggccgctgcggctgctggcaactgcgctcggggcgatgcgggcgacgcggcagcccctggcaccgcgcctgggtccggcctgatgctgcctccgcctcccaagcgccctcgcgtcagcttcgcggacgacgactcggcggcggccggtggtgggggcggcggggctccgcggtcggcgctggagactgtgattggcttcacagacggcaaggacttcgtccgggccagcaagctgatctaaggatcc

SEQ ID NO: 268 - DNA, 789 bp, artificial sequence
  codon optimized
attcctgctcaaaatgagaagccacacaagggggtgctgctgcaagcagggtggctgcacctgtcctcgcccgaggagcggaaggctgctgaggtgcaagcggaggcgacccgcctgatgccgcctccacgtctgcccggtgctgccagcgcccctgctgtggagatggtcccgactgcggacggtccaccgaagtgccccgtgccgtcctgggctggcgagccgccagcaggctcccgcctgctggtgtacaaggagggcacggtgatccaggacatcgccctgggcaaggtggtgactgtgttcggccgcgtcccagagctggcggacgtggtgctggaccacccctcgatttcgcgccaacacgccaccgcggcctggcaccctggccgcgctgcctggctcctgaccgacctcggctccacacacggcacatgggtgggcgactcgcggctgggcaagaacgagccggccgagctggtgcccggcgtcgaggtccgctttgcggccagcacgcgccgctacaagctcgccgctcccctgggcggcaaggccgctgcggctgctggcaactgcgctcggggcgatgcgggcgacgcggcagcccctggcaccgcgcctgggtccggcctgatgctgcctccgcctcccaagcgccctcgcgtcagcttcgcggacgacgactcggcggcggccggtggtgggggcggcggggctccgcggtcggcgctggagactgtgattggcttcacagacggcaaggacttcgtccgggccagcaagctgatc

SEQ ID NO: 269 - DNA, 789 bp, Chlamydomonas reinhardtii
atacctgcacaaaacgaaaagccccacaagggcgtgttgctgcaggcgggctggctgcatttgtcaagtccggaagaacggaaagccgctgaggttcaggccgaggcgacccggctcatgcctccgccgcgcctgccgggcgccgcctccgccccagctgtggaaatggttcccaccgctgacggcccccccaagtgccccgtgccgtcatgggcaggagagccgccggcggggtcgcggctgcttgtgtacaaggaggggacagtcatccaggacattgcactgggcaaggtggtgactgtgttcggccgtgtgccggagctggcggacgtggtgctggaccacccctccatcagccgccagcacgccaccgccgcctggcacccgggccgcgccgcctggctgctcaccgacctgggctccacacacggcacctgggtgggcgacagccgcctgggcaagaacgagcccgccgagctggtgcctggtgtggaggtccgcttcgccgcctccacccgcagatacaagctggcggcgccgctgggcggcaaggcggcggcggcggcggggaactgtgcccgtggggatgcgggtgacgcagccgccccgggtacggcgccagggtcggggctgatgctgccaccgccgccgaagcggccgcgagtatctttcgccgacgacgactcggccgctgcgggcggcggtggcggcggtgcgccgcggtctgcgctggagactgtcattggcttcacggacggcaaggactttgtgcgagcttcaaagctcatc

SEQ ID NO: 270 - protein, 263 aa, Chlamydomonas reinhardtii
IPAQNEKPHKGVLLQAGWLHLSSPEERKAAEVQAEATRLMPPPRLPGAASAPAVEMVPTADGPPKCPVPSWAGEPPAGSRLLVYKEGTVIQDIALGKVVTVFGRVPELADVVLDHPSISRQHATAAWHPGRAAWLLTDLGSTHGTWVGDSRLGKNEPAELVPGVEVRFAASTRRYKLAAPLGGKAAAAAGNCARGDAGDAAAPGTAPGSGLMLPPPPKRPRVSFADDDSAAAGGGGGGAPRSALETVIGFTDGKDFVRASKLI

SEQ ID NO: 271 - DNA, 1011 bp, Chlamydomonas reinhardtii
atgtgccccctcacgtcctccattgagtacctgtgcgcgggtgacgaggtcagcaccgtactgagcggcgacacaggagagccggtccccggcggcattgccgtacagtccaactgggggcccagctcgcgctactgccacgtcaacgtcaccatccagggcctggacgcaatgtgcacgggcccgcgtacgaatcccttcaacggccccgctggctgccaaggcaatgacaatgtcgacaatgagtgcctctggagcatcaaggcgccgcggcccggaggacctggctgggccggcgaggtgtgcacgaaacccccgccgcccgctcctcctcccatgcagcccaatgcgccgtcgaaaaagcgcccgccgcctcgcccagccaagccaggccagcgcgcccccaatgtaccgccatcaccatcaccgccgccgcagcagtacatccccttccccttttgcgcatgcaaaaaacgcaacatcaagaacacaccataccgattcgacttcatctctagcacgccactgcccaccttgtctgatggcaagccgcgtgtgcggcactgcttcaacattgatacggtggcctgtgacgccacccattcctgctgcaacatgggtctcaagaagattgagatcttcgccaacaacgactgccgttcgtccgtcaagctcgccctgcttgctggccagagtattagctgggctttcacccaggacacgtacaacggcaacacgtacaccaccttcaaattccccaacctaatgctgagccgtgcggacgtgggcaagggcatgagcctgtgctttattcttacggatacctgctccaagctggagaatttctgctacgacggcaagaataactcctgccgagtcactttcttcagcgtggacgaaagctgctgccccaccggaccagccagcttggaggcgagcacacccgagtttgagacggcagcaccccctcctgatgcggtcactgtggatccaacaggaggccatcgttaa

SEQ ID NO: 272 - protein, 336 aa, Chlamydomonas reinhardtii
MCPLTSSIEYLCAGDEVSTVLSGDTGEPVPGGIAVQSNWGPSSRYCHVNVTIQGLDAMCTGPRTNPFNGPAGCQGNDNVDNECLWSIKAPRPGGPGWAGEVCTKPPPPAPPPMQPNAPSKKRPPPRPAKPGQRAPNVPPSPSPPPQQYIPFPFCACKKRNIKNTPYRFDFISSTPLPTLSDGKPRVRHCFNIDTVACDATHSCCNMGLKKIEIFANNDCRSSVKLALLAGQSISWAFTQDTYNGNTYTTFKFPNLMLSRADVGKGMSLCFILTDTCSKLENFCYDGKNNSCRVTFFSVDESCCPTGPASLEASTPEFETAAPPPDAVTVDPTGGHR

SEQ ID NO: 273 - DNA, 1026 bp, artificial sequence
  codon optimized
catatgctcgagtgtcctctgacttccagcattgagtatctctgcgcgggtgatgaggtgtccaccgtgctgtccggcgacactggtgagccggtgccgggcggcatcgccgtgcagtcgaactggggcccgtcgagccgctactgccacgtgaacgtgacaatccagggcctcgacgcgatgtgcaccggccctcgtacgaacccgttcaacggccctgccggctgccaggggaacgacaacgtggataacgagtgcctgtggtcgatcaaggcacctcgccctgggggcccaggctgggccggtgaggtgtgcaccaagccccctccgccggctccgcctccgatgcagccgaacgccccctcgaagaagcgcccaccgccacgtccggcgaagcccggccagcgggcacccaacgtgcccccatcgccaagccctccccctcagcagtacattcccttccccttctgcgcttgcaagaagcgcaacatcaagaacaccccctatcggtttgacttcatcagctcgacgccactgccgacgctgtcggacggcaagccccgcgtgcgccactgcttcaacattgacaccgtggcttgcgatgccacccacagctgctgcaacatgggcctgaagaagatcgagattttcgcgaacaacgactgccggtcgtcggtgaagctcgccctgctggcgggccagagcatctcctgggcgttcacccaggacacctacaacggcaacacctacacgacgttcaagttccctaacctgatgctcagccgcgccgacgtgggcaagggtatgagcctgtgtttcatcctgaccgacacatgctccaagctggagaacttctgttacgacggcaagaacaacagctgccgcgtgaccttcttcagcgtggacgagtcgtgctgcccgactggccccgcaagcctggaggcgtccaccccggagttcgagacggctgctccgcctcccgacgcggtgacggtggacccgacaggcggccatcgctaaggatcc

SEQ ID NO: 274 - DNA, 1005 bp, artificial sequence
  codon optimized
tgtcctctgacttccagcattgagtatctctgcgcgggtgatgaggtgtccaccgtgctgtccggcgacactggtgagccggtgccgggcggcatcgccgtgcagtcgaactggggcccgtcgagccgctactgccacgtgaacgtgacaatccagggcctcgacgcgatgtgcaccggccctcgtacgaacccgttcaacggccctgccggctgccaggggaacgacaacgtggataacgagtgcctgtggtcgatcaaggcacctcgccctgggggcccaggctgggccggtgaggtgtgcaccaagccccctccgccggctccgcctccgatgcagccgaacgccccctcgaagaagcgcccaccgccacgtccggcgaagcccggccagcgggcacccaacgtgcccccatcgccaagccctccccctcagcagtacattcccttccccttctgcgcttgcaagaagcgcaacatcaagaacaccccctatcggtttgacttcatcagctcgacgccactgccgacgctgtcggacggcaagccccgcgtgcgccactgcttcaacattgacaccgtggcttgcgatgccacccacagctgctgcaacatgggcctgaagaagatcgagattttcgcgaacaacgactgccggtcgtcggtgaagctcgccctgctggcgggccagagcatctcctgggcgttcacccaggacacctacaacggcaacacctacacgacgttcaagttccctaacctgatgctcagccgcgccgacgtgggcaagggtatgagcctgtgtttcatcctgaccgacacatgctccaagctggagaacttctgttacgacggcaagaacaacagctgccgcgtgaccttcttcagcgtggacgagtcgtgctgcccgactggccccgcaagcctggaggcgtccaccccggagttcgagacggctgctccgcctcccgacgcggtgacggtggacccgacaggcggccatcgc

SEQ ID NO: 275 - DNA, 1005 bp, Chlamydomonas reinhardtii
tgccccctcacgtcctccattgagtacctgtgcgcgggtgacgaggtcagcaccgtactgagcggcgacacaggagagccggtccccggcggcattgccgtacagtccaactgggggcccagctcgcgctactgccacgtcaacgtcaccatccagggcctggacgcaatgtgcacgggcccgcgtacgaatcccttcaacggccccgctggctgccaaggcaatgacaatgtcgacaatgagtgcctctggagcatcaaggcgccgcggcccggaggacctggctgggccggcgaggtgtgcacgaaacccccgccgcccgctcctcctcccatgcagcccaatgcgccgtcgaaaaagcgcccgccgcctcgcccagccaagccaggccagcgcgcccccaatgtaccgccatcaccatcaccgccgccgcagcagtacatccccttccccttttgcgcatgcaaaaaacgcaacatcaagaacacaccataccgattcgacttcatctctagcacgccactgcccaccttgtctgatggcaagccgcgtgtgcggcactgcttcaacattgatacggtggcctgtgacgccacccattcctgctgcaacatgggtctcaagaagattgagatcttcgccaacaacgactgccgttcgtccgtcaagctcgccctgcttgctggccagagtattagctgggctttcacccaggacacgtacaacggcaacacgtacaccaccttcaaattccccaacctaatgctgagccgtgcggacgtgggcaagggcatgagcctgtgctttattcttacggatacctgctccaagctggagaatttctgctacgacggcaagaataactcctgccgagtcactttcttcagcgtggacgaaagctgctgccccaccggaccagccagcttggaggcgagcacacccgagtttgagacggcagcaccccctcctgatgcggtcactgtggatccaacaggaggccatcgt

SEQ ID NO: 276 - protein, 335 aa, Chlamydomonas reinhardtii
CPLTSSIEYLCAGDEVSTVLSGDTGEPVPGGIAVQSNWGPSSRYCHVNVTIQGLDAMCTGPRTNPFNGPAGCQGNDNVDNECLWSIKAPRPGGPGWAGEVCTKPPPPAPPPMQPNAPSKKRPPPRPAKPGQRAPNVPPSPSPPPQQYIPFPFCACKKRNIKNTPYRFDFISSTPLPTLSDGKPRVRHCFNIDTVACDATHSCCNMGLKKIEIFANNDCRSSVKLALLAGQSISWAFTQDTYNGNTYTTFKFPNLMLSRADVGKGMSLCFILTDTCSKLENFCYDGKNNSCRVTFFSVDESCCPTGPASLEASTPEFETAAPPPDAVTVDPTGGHR

SEQ ID NO: 277 - DNA, 1194 bp, Chlamydomonas reinhardtii
atgtttggatacccgcatggttgcagggaggggcagtggtgcaagctgcgcccggatctggacgcgctcggcgtgaagctcgacgccaacggcatcgtcacgcacctcaagtacgccctcgcgccgcccaagccgcccagcgcacacctcgtggaggtgatgcaggctggtgccatccgcgacatcctgatgggcatgggctgggccgcgctgccggagcagcaggttcgggtgttcgacattgaccttgccgctgcggatgcgctcgcgctggaggccctgcaagggtgggcacgcatagcgggggtggcgctgcaaggcgaaggccagaagctggtgcggcgccagcaggggcttagcaccggcggcgacaaaacctcgtggtaccagcagtggttcgcggagtgcccgttcggcctgctggatgtcaccgggcaggacgtgctgggcaggtacgtcaaccagatcatctacgagtgcgaggcggagcattatccattcagcatcgaggaggcgctggaggagatgtgtacggcggtgtgggaggcggcggtgcaggttgcgccgtacctgaccaagtacagggatgagttcttgtctgcctgggggcgccaggcgatgtacggcgacacggcaactaacctcgtgagcatgaccaagaactgcgcggtgtcgttgcacttcgacacaacggacgggccgtacagcatcatgctgtggcgccacaacggtgccggcagcctggacggcgggcatttcttgatgcctggcgcctccatcaaggtgctgccgaccgacatgacgatcgtggtgctggctgctggcatggtcacgcatgggacggcgcccgtgctggagtccactggcgacgcgcggcggtatggctactcgcatttcctgcgtgtgccggccatggagcgtgtggcgcggctgatcaaggcatctggcggaaagaagaagatggaggagctgcaggtacagggcatgaagcgcgtgttggctgcacgtacagcagcggatcggaaggcgcggcgggatgaaatccagaagcagcgggacgagctcctgaagagcgcgctggacggcgaggcgctgcccgagggcgagcatttagcgtttgctgtgcgagggttgaagtggcaccgggacattgtgaagtgcctggtatggcaggacttcaagggcaagtcctga

SEQ ID NO: 278 - protein, 397 aa, Chlamydomonas reinhardtii
MFGYPHGCREGQWCKLRPDLDALGVKLDANGIVTHLKYALAPPKPPSAHLVEVMQAGAIRDILMGMGWAALPEQQVRVFDIDLAAADALALEALQGWARIAGVALQGEGQKLVRRQQGLSTGGDKTSWYQQWFAECPFGLLDVTGQDVLGRYVNQIIYECEAEHYPFSIEEALEEMCTAVWEAAVQVAPYLTKYRDEFLSAWGRQAMYGDTATNLVSMTKNCAVSLHFDTTDGPYSIMLWRHNGAGSLDGGHFLMPGASIKVLPTDMTIVVLAAGMVTHGTAPVLESTGDARRYGYSHFLRVPAMERVARLIKASGGKKKMEELQVQGMKRVLAARTAADRKARRDEIQKQRDELLKSALDGEALPEGEHLAFAVRGLKWHRDIVKCLVWQDFKGKS

SEQ ID NO: 279 - DNA, 1203 bp, artificial sequence
  codon optimized
ctcgagttcggctacccgcacggctgccgcgagggccaatggtgcaagctgcgcccagacctggacgcgctcggcgtgaagctggacgccaacggcattgtcacccacctcaagtacgcgctggccccacccaagccccctagcgcgcacctggtggaggtgatgcaggcgggtgctatccgcgatattctgatggggatgggctgggccgcgctgcccgagcaacaagtgcgcgtgttcgacattgatctggccgctgccgacgccctggccctggaggctctccagggctgggcccgcatcgcgggcgtcgcactccagggcgagggccaaaagctggtgcgtcgccagcaagggctgagcacgggtggtgacaagacctcgtggtatcagcagtggttcgccgagtgcccctttggcctgctggacgtgaccggccaggacgtgctgggccggtacgtgaaccagatcatctacgagtgcgaggccgagcattaccccttctccatcgaggaggcactggaggagatgtgcaccgcggtctgggaggcagctgtccaggtggcaccgtacctgaccaagtaccgtgatgagttcctgagcgcctggggccgtcaggcgatgtacggtgacaccgccacgaatctggtcagcatgaccaagaattgcgcggtgagcctgcacttcgacactacagatggcccctattccatcatgctgtggcgtcacaacggcgccggctcgctggacgggggccatttcctcatgcctggcgcgtccatcaaggtgctgcccacggacatgacgatcgtggtgctcgcggcaggcatggtcactcacggtacggcgccagtgctggagagcaccggggatgcgcgccgctatggttacagccacttcctgcgtgtccccgcaatggagcgggtggcccgcctgattaaggcctcgggcggcaagaagaagatggaggagctgcaagtgcaggggatgaagcgggtgctggcggctcgcacggcagccgaccgcaaggctcgccgcgatgagatccagaagcagcgtgacgagctgctgaagagcgcactggacggcgaggccctgcccgagggcgagcacctggcgttcgcggtgcgcggcctgaagtggcaccgcgacatcgtgaagtgtctggtgtggcaggacttcaagggcaagtcgtaaggatcc

SEQ ID NO: 280 - DNA, 1188 bp, artificial sequence
  codon optimized
ttcggctacccgcacggctgccgcgagggccaatggtgcaagctgcgcccagacctggacgcgctcggcgtgaagctggacgccaacggcattgtcacccacctcaagtacgcgctggccccacccaagccccctagcgcgcacctggtggaggtgatgcaggcgggtgctatccgcgatattctgatggggatgggctgggccgcgctgcccgagcaacaagtgcgcgtgttcgacattgatctggccgctgccgacgccctggccctggaggctctccagggctgggcccgcatcgcgggcgtcgcactccagggcgagggccaaaagctggtgcgtcgccagcaagggctgagcacgggtggtgacaagacctcgtggtatcagcagtggttcgccgagtgcccctttggcctgctggacgtgaccggccaggacgtgctgggccggtacgtgaaccagatcatctacgagtgcgaggccgagcattaccccttctccatcgaggaggcactggaggagatgtgcaccgcggtctgggaggcagctgtccaggtggcaccgtacctgaccaagtaccgtgatgagttcctgagcgcctggggccgtcaggcgatgtacggtgacaccgccacgaatctggtcagcatgaccaagaattgcgcggtgagcctgcacttcgacactacagatggcccctattccatcatgctgtggcgtcacaacggcgccggctcgctggacgggggccatttcctcatgcctggcgcgtccatcaaggtgctgcccacggacatgacgatcgtggtgctcgcggcaggcatggtcactcacggtacggcgccagtgctggagagcaccggggatgcgcgccgctatggttacagccacttcctgcgtgtccccgcaatggagcgggtggcccgcctgattaaggcctcgggcggcaagaagaagatggaggagctgcaagtgcaggggatgaagcgggtgctggcggctcgcacggcagccgaccgcaaggctcgccgcgatgagatccagaagcagcgtgacgagctgctgaagagcgcactggacggcgaggccctgcccgagggcgagcacctggcgttcgcggtgcgcggcctgaagtggcaccgcgacatcgtgaagtgtctggtgtggcaggacttcaagggcaagtcg

SEQ ID NO: 281 - DNA, 1188 bp, Chlamydomonas reinhardtii
tttggatacccgcatggttgcagggaggggcagtggtgcaagctgcgcccggatctggacgcgctcggcgtgaagctcgacgccaacggcatcgtcacgcacctcaagtacgccctcgcgccgcccaagccgcccagcgcacacctcgtggaggtgatgcaggctggtgccatccgcgacatcctgatgggcatgggctgggccgcgctgccggagcagcaggttcgggtgttcgacattgaccttgccgctgcggatgcgctcgcgctggaggccctgcaagggtgggcacgcatagcgggggtggcgctgcaaggcgaaggccagaagctggtgcggcgccagcaggggcttagcaccggcggcgacaaaacctcgtggtaccagcagtggttcgcggagtgcccgttcggcctgctggatgtcaccgggcaggacgtgctgggcaggtacgtcaaccagatcatctacgagtgcgaggcggagcattatccattcagcatcgaggaggcgctggaggagatgtgtacggcggtgtgggaggcggcggtgcaggttgcgccgtacctgaccaagtacagggatgagttcttgtctgcctgggggcgccaggcgatgtacggcgacacggcaactaacctcgtgagcatgaccaagaactgcgcggtgtcgttgcacttcgacacaacggacgggccgtacagcatcatgctgtggcgccacaacggtgccggcagcctggacggcgggcatttcttgatgcctggcgcctccatcaaggtgctgccgaccgacatgacgatcgtggtgctggctgctggcatggtcacgcatgggacggcgcccgtgctggagtccactggcgacgcgcggcggtatggctactcgcatttcctgcgtgtgccggccatggagcgtgtggcgcggctgatcaaggcatctggcggaaagaagaagatggaggagctgcaggtacagggcatgaagcgcgtgttggctgcacgtacagcagcggatcggaaggcgcggcgggatgaaatccagaagcagcgggacgagctcctgaagagcgcgctggacggcgaggcgctgcccgagggcgagcatttagcgtttgctgtgcgagggttgaagtggcaccgggacattgtgaagtgcctggtatggcaggacttcaagggcaagtcc

SEQ ID NO: 282 - protein, 396 aa, Chlamydomonas reinhardtii
FGYPHGCREGQWCKLRPDLDALGVKLDANGIVTHLKYALAPPKPPSAHLVEVMQAGAIRDILMGMGWAALPEQQVRVFDIDLAAADALALEALQGWARIAGVALQGEGQKLVRRQQGLSTGGDKTSWYQQWFAECPFGLLDVTGQDVLGRYVNQIIYECEAEHYPFSIEEALEEMCTAVWEAAVQVAPYLTKYRDEFLSAWGRQAMYGDTATNLVSMTKNCAVSLHFDTTDGPYSIMLWRHNGAGSLDGGHFLMPGASIKVLPTDMTIVVLAAGMVTHGTAPVLESTGDARRYGYSHFLRVPAMERVARLIKASGGKKKMEELQVQGMKRVLAARTAADRKARRDEIQKQRDELLKSALDGEALPEGEHLAFAVRGLKWHRDIVKCLVWQDFKGKS

SEQ ID NO: 283 - DNA, 177 bp, Chlamydomonas reinhardtii
atggtgcaggcattccgcgatcatacggacggcggcattgtgtttggcgatgcgggccccaacaagttgggggtgcgcacagtggccggcaaggtggcctgcgttgccccgttcggggggggatccagcgcgctggtgaatcagggcgcagagtgcacatgtgtcacttgcacgtag

SEQ ID NO: 284 - protein, 58 aa, Chlamydomonas reinhardtii
MVQAFRDHTDGGIVFGDAGPNKLGVRTVAGKVACVAPFGGGSSALVNQGAECTCVTCT

SEQ ID NO: 285 - DNA, 186 bp, artificial sequence
  codon optimized
ctcgaggtgcaagcatttcgtgatcatacagatggtggcattgtgtttggcgacgctggccctaacaagctgggcgtgcgcaccgtcgcgggcaaggtggcgtgcgtggccccctttggcggtggcagcagcgctctcgtgaaccagggcgcggagtgcacgtgcgtgacttgcacctaaggatcc

SEQ ID NO: 286 - DNA, 171 bp, artificial sequence
  codon optimized
gtgcaagcatttcgtgatcatacagatggtggcattgtgtttggcgacgctggccctaacaagctgggcgtgcgcaccgtcgcgggcaaggtggcgtgcgtggccccctttggcggtggcagcagcgctctcgtgaaccagggcgcggagtgcacgtgcgtgacttgcacc

SEQ ID NO: 287 - DNA, 171 bp, Chlamydomonas reinhardtii
gtgcaggcattccgcgatcatacggacggcggcattgtgtttggcgatgcgggccccaacaagttgggggtgcgcacagtggccggcaaggtggcctgcgttgccccgttcggggggggatccagcgcgctggtgaatcagggcgcagagtgcacatgtgtcacttgcacg

SEQ ID NO: 288 - protein, 57 aa, Chlamydomonas reinhardtii
VQAFRDHTDGGIVFGDAGPNKLGVRTVAGKVACVAPFGGGSSALVNQGAECTCVTCT

SEQ ID NO: 289 - DNA, 459 bp, Chlamydomonas reinhardtii
atgcaggatggggaccggcgtcactggttttgggactgcactgttgcgctgtcactgcgggaaagtatggggatggctatgggtttcctgccggaggaggctctaagtgccttctctcgtgaggagttgtggttagtgcgcccgcctgcggggcttgcgccacctgtgtgggatgtggtgtgtctcgctgctatgtctgccctggactttggtcggcagcgtatggttatggccgggttggcggcgcgagcgaagctgccgtcggcccgggtgctgagcattggacttgccgtcgtagctgacttctggggtcgtctccagacgtttgtgactctgggtatcaggccaaagggttgggacactgtgccgtctgcgcatcctttcatctctcgggctgttggtgacggcatggttttgcgcttgccgtatgacgctgattacccgcctccctcgccgtga

SEQ ID NO: 290 - protein, 152 aa, Chlamydomonas reinhardtii
MQDGDRRHWFWDCTVALSLRESMGMAMGFLPEEALSAFSREELWLVRPPAGLAPPVWDVVCLAAMSALDFGRQRMVMAGLAARAKLPSARVLSIGLAVVADFWGRLQTFVTLGIRPKGWDTVPSAHPFISRAVGDGMVLRLPYDADYPPPSP

SEQ ID NO: 291 - DNA, 468 bp, artificial sequence
  codon optimized
ctcgagcaagacggggaccggcggcactggtgctgggattgcacggtggcgctgtcgctccgcgagaacatggggctcgcgatgggcttcctccccgaggaggctctgagcgcgttctcccgcgaggagctgtggctggtccgcccgcctgcgggtctggctcccccggtgtgggacgtggtctgcctggccgccatgtccgccctggactttggccggcagcgcatggtcatggctggcctggcggcacgcgcgaagctgccctcggcacgcgtgctgagcattggtctggccgtcgtggctgacttctggggccgtctgcaaacctttgtgactctgggcatccgccctaaggggtgggacaccgtgccgtccgcacacccgttcatctcccgtgccgtgggcgacggcatggtgctgcgcctgccgtacgacgctgattacccaccgccgtccccgtaaggatcc

SEQ ID NO: 292 - DNA, 453 bp, artificial sequence
  codon optimized
caagacggggaccggcggcactggtgctgggattgcacggtggcgctgtcgctccgcgagaacatggggctcgcgatgggcttcctccccgaggaggctctgagcgcgttctcccgcgaggagctgtggctggtccgcccgcctgcgggtctggctcccccggtgtgggacgtggtctgcctggccgccatgtccgccctggactttggccggcagcgcatggtcatggctggcctggcggcacgcgcgaagctgccctcggcacgcgtgctgagcattggtctggccgtcgtggctgacttctggggccgtctgcaaacctttgtgactctgggcatccgccctaaggggtgggacaccgtgccgtccgcacacccgttcatctcccgtgccgtgggcgacggcatggtgctgcgcctgccgtacgacgctgattacccaccgccgtccccg

SEQ ID NO: 293 - DNA, 453 bp, Chlamydomonas reinhardtii
caggatggggaccggcgtcactggttttgggactgcactgttgcgctgtcactgcgggaaagtatggggatggctatgggtttcctgccggaggaggctctaagtgccttctctcgtgaggagttgtggttagtgcgcccgcctgcggggcttgcgccacctgtgtgggatgtggtgtgtctcgctgctatgtctgccctggactttggtcggcagcgtatggttatggccgggttggcggcgcgagcgaagctgccgtcggcccgggtgctgagcattggacttgccgtcgtagctgacttctggggtcgtctccagacgtttgtgactctgggtatcaggccaaagggttgggacactgtgccgtctgcgcatcctttcatctctcgggctgttggtgacggcatggttttgcgcttgccgtatgacgctgattacccgcctccctcgccg

SEQ ID NO: 294 - protein, 151 aa, Chlamydomonas reinhardtii
QDGDRRHWFWDCTVALSLRESMGMAMGFLPEEALSAFSREELWLVRPPAGLAPPVWDVVCLAAMSALDFGRQRMVMAGLAARAKLPSARVLSIGLAVVADFWGRLQTFVTLGIRPKGWDTVPSAHPFISRAVGDGMVLRLPYDADYPPPSP

SEQ ID NO: 295 - DNA, 201 bp, Chlamydomonas reinhardtii
atgggcggccccccgcactgcaaccatccaagtccccgcaccacccgttgcaccgactccgccgtagcactcacgacgtcggccaccgggagcggcagcgcggcggcaggcccccgagccagctgggccgcacgcgcggcggcagcccagccagcgggaagaagggccaccgccgccgcaaaccggtcggccgcctcttga

SEQ ID NO: 296 - protein, 66 aa, Chlamydomonas reinhardtii
MGGPPHCNHPSPRTTRCTDSAVALTTSATGSGSAAAGPRASWAARAAAAQPAGRRATAAANRSAAS

SEQ ID NO: 297 - DNA, 207 bp, artificial sequence
  codon optimized
ctcgagggtggtcctcctcattgtaatcatcctagccctcgtactactcgttgcaccgactccgcggtggctctgaccacgtcggccacgggcagcggctccgctgctgcgggtccgcgggcatcctgggccgctcgcgccgcagcggctcagccagccggtcgccgcgctactgcggctgccaaccgcagcgccgccagcaccggt

SEQ ID NO: 298 - DNA, 195 bp, artificial sequence
  codon optimized
ggtggtcctcctcattgtaatcatcctagccctcgtactactcgttgcaccgactccgcggtggctctgaccacgtcggccacgggcagcggctccgctgctgcgggtccgcgggcatcctgggccgctcgcgccgcagcggctcagccagccggtcgccgcgctactgcggctgccaaccgcagcgccgccagc

SEQ ID NO: 299 - DNA, 195 bp, Chlamydomonas reinhardtii
ggcggccccccgcactgcaaccatccaagtccccgcaccacccgttgcaccgactccgccgtagcactcacgacgtcggccaccgggagcggcagcgcggcggcaggcccccgagccagctgggccgcacgcgcggcggcagcccagccagcgggaagaagggccaccgccgccgcaaaccggtcggccgcctct

SEQ ID NO: 300 - protein, 65 aa, Chlamydomonas reinhardtii
GGPPHCNHPSPRTTRCTDSAVALTTSATGSGSAAAGPRASWAARAAAAQPAGRRATAAANRSAAS

(19) United States
(12) Patent Application Publication
(10) Pub. No.: US 2016/0194649 A1
(43) Pub. Date: Jul. 7, 2016

(54) LIPID AND GROWTH TRAIT GENES

(71) Applicant: Sapphire Energy, Inc., San Diego, CA (US)

(72) Inventor: CHRISTOPHER YOHN, San Diego, CA (US)

(21) Appl. No.: 14/378,931

(22) PCT Filed: Feb. 25, 2013
(86) PCT No.: PCT/US2013/027661

§ 371 Date: Aug. 14, 2014

Related U.S. Application Data
(60) Provisional application No. 61/602,892, filed on Feb. 24, 2012.

(51) Int. Cl. C12N 15/82; C07K 14/405; C12P 7/64

(52) U.S. Cl. CPC C12N 15/8247; C12P 7/6463; C07K 14/405

(57) ABSTRACT

The present disclosure provides novel lipid and growth genes that when over expressed in an organism results in a change in the lipid profile, and/or lipid content, and/or growth of the organism. The present disclosure also describes organisms expressing the genes, and methods of using the novel genes to change the lipid content, lipid profile or growth of an organism.

### CROSS REFERENCE TO RELATED APPLICATIONS

This application claims the benefit of U.S. Provisional Patent Application Ser. No. 61/602,892, filed Feb. 24, 2012, of which is herein incorporated by reference in its entirety for all purposes.

### BACKGROUND

Microalgae represent a diverse group of micro-organisms adapted to various ecological habitats (for example, as described in Hu et al., Plant J (2008) vol. 54 (4) pp. 621-639). Many microalgae have the ability to produce substantial amounts (for example, 20-50% dry cell weight) of lipids, such as triacylglycerols (TAGs) and diacylglycerols (DAGs), as storage lipids under stress conditions, such as nitrogen starvation. Under nitrogen starvation many microalgae exhibit decreased growth rate and break down of photosynthetic components, such as chlorophyll.

Fatty acids, the building blocks for TAGs and all other cellular lipids, are synthesized in the chloroplast using a single set of enzymes, in which acetyl CoA carboxylase (ACCase) is key in regulating fatty acid synthesis rates. However, the expression of genes involved in fatty acid synthesis is poorly understood in microalgae. Synthesis and sequestration of TAGs into cytosolic lipid bodies appears to be a protective mechanism by which algal cells cope with stress conditions.

Little is known about the regulation of lipids, such as TAG formation, at the molecular or cellular level. At the biochemical level, available information about fatty acid and TAG synthetic pathways in algae is still fragmentary. Knowledge regarding both the regulatory and structural genes involved in these pathways and the potential interactions between the pathways is lacking. Because fatty acids are common precursors for the synthesis of both membrane lipids and TAGs, how the algal cell coordinates the distribution of the precursors to the two distinct destinations or the inter-conversion between the two types of lipids needs to be elucidated. Many fundamental biological questions relating to the biosynthesis and regulation of fatty acids and lipids in algae need to be answered.

Much research has been conducted over the last few decades regarding using microalgae as an alternative and renewable source of lipid-rich biomass feedstock for biofuels. Microalgae are an attractive model in that they are capable of producing substantial amounts of lipids such as TAGs and DAGs under stress conditions, such as nitrogen starvation. However, a decrease in growth of the microalgae under nitrogen starvation makes it harder to use microalgae in the large scale production of biofuels. While algae provide the natural raw material in the form of lipid-rich feedstock, our understanding of the details of lipid metabolism in order to enable the manipulation of the process physiologically and genetically is lacking.

Thus, a need exists to better understand the regulation of lipids, such as TAGs and DAGs, in algae at the molecular level. Furthermore, it would be useful to genetically manipulate algae such that the algae are capable of producing substantial amounts of lipids without decreased growth rate and the breakdown of algal components, such as chlorophyll. The present disclosure meets this need by providing novel genes that when used to transform algae results in the desired phenotype.

In addition, microalgae and biofuels hold a promising partnership, but there is a need for an order of magnitude increase in productivity that will require the development of new technologies, for example, the transformation of cells as well as identification of trait genes for improving strains. Improved strains are needed to increase volumetric productivity and to produce desired levels of lipids.

Optimizing the growth of algae in, for example, open ponds is a key component of reaching economic viability and remains a challenge for the industry. Identifying species that grow well under these conditions is a focus of ongoing research. Algae can grow in a wide variety of temperatures, with growth being limited primarily by nutrient availability and light. Growth rates are often limited by light penetration into the ponds from both self-shading and light absorption by the water, and these constraints are major determining factors of pond depth (Mayfield, S., et al., Biofuels (2010) 1 (5): 763-784).

Genetic and metabolic engineering are likely to have the greatest impact on improving the economics of production of microalgae. Molecular engineering of algae can be used, for example, to increase photosynthetic efficiency to increase biomass yield on light, enhance biomass growth/growth rate, and increase oil content in the biomass.

Therefore, it would also be beneficial to genetically manipulate algae such that the algae have increased growth resulting in an increase in algal biomass. The present disclosure meets this need by providing novel genes that when used to transform algae results in the desired phenotype.

### SUMMARY

Provided herein is an isolated polynucleotide, comprising: (a) a nucleic acid sequence of SEQ ID NO: 131, 65, 77, 83, 89, 95, 101, 107, 113, 119, 125, 137, 143, 149, 155, 161, 167 or 173; (b) a nucleotide sequence with at least 80%, at least 85%, at least 90%, at least 95%, at least 98%, or at least 99% sequence identity to the nucleic acid sequence of SEQ ID NO: 131, 65, 77, 83, 89, 95, 101, 107, 113, 119, 125, 137, 143, 149, 155, 161, 167 or 173; (c) a nucleic acid sequence of SEQ ID NO: 130, 64, 76, 82, 88, 94, 100, 106, 112, 118, 124, 136, 142, 148, 154, 160, 166, or 172; or (d) a nucleotide sequence with at least 80%, at least 85%, at least 90%, at least 95%, at least 98%, or at least 99% sequence identity to the nucleic acid sequence of 130, 64, 76, 82, 88, 94, 100, 106, 112, 118, 124, 136, 142, 148, 154, 160, 166, or 172. In one embodiment, an organism is transformed with the isolated polynucleotide. In another embodiment, a vector comprises the isolated polynucleotide. In yet another embodiment, the vector further comprises a 5′ regulatory region. In one embodiment, the 5′ regulatory region further comprises a promoter. In other embodiments, the promoter is a constitutive promoter or the promoter is an inducible promoter. In some embodiments, the inducible promoter is a light inducible promoter, a nitrate inducible promoter, or a heat responsive promoter. In one embodiment, the vector further comprises a 3′ regulatory region.

Also provided herein is an isolated polynucleotide encoding a protein comprising, (a) an amino acid sequence of SEQ ID NO: 132, 66, 78, 84, 90, 96, 102, 108, 114, 120, 126, 138, 144, 150, 156, 162, 168, or 174; or (b) a homolog of the amino acid sequence of (a), wherein the homolog has at least 80%, at least 85%, at least 90%, at least 95%, at least 98%, or at least 99% sequence identity to the amino acid sequence of SEQ ID NO: 132, 66, 78, 84, 90, 96, 102, 108, 114, 120, 126, 138, 144, 150, 156, 162, 168, or 174. In one embodiment, the organism is transformed with the isolated polynucleotide and the protein is expressed.

Also provided is a photosynthetic organism transformed with an isolated polynucleotide comprising: (a) a nucleic acid sequence of SEQ ID NO: 131, 65, 77, 83, 89, 95, 101, 107, 113, 119, 125, 137, 143, 149, 155, 161, 167 or 173; (b) a nucleotide sequence with at least 80%, at least 85%, at least 90%, at least 95%, at least 98%, or at least 99% sequence identity to the nucleic acid sequence of SEQ ID NO: 131, 65, 77, 83, 89, 95, 101, 107, 113, 119, 125, 137, 143, 149, 155, 161, 167 or 173; (c) a nucleic acid sequence of SEQ ID NO: 130, 64, 76, 82, 88, 94, 100, 106, 112, 118, 124, 136, 142, 148, 154, 160, 166, or 172; or (d) a nucleotide sequence with at least 80%, at least 85%, at least 90%, at least 95%, at least 98%, or at least 99% sequence identity to the nucleic acid sequence of 130, 64, 76, 82, 88, 94, 100, 106, 112, 118, 124, 136, 142, 148, 154, 160, 166, or 172; wherein the transformed organism's lipid content or profile is different than an untransformed organism's lipid content or profile or a second transformed organism's lipid content or profile. In some embodiments, the difference is an increase or decrease in one or more of a heme, a polar lipid, a chlorophyll breakdown product, pheophytin, a digalactosyl diacylglycerol (DGDG), a triacylglycerol, a diacylglycerol, a monoacylglycerol, a sterol, a sterol ester, a wax ester, a tocopherol, a fatty acid, phosphatidic acid, lysophosphatidic acid, phosphatidyl glycerol, cardiolipin (diphosphatidylglycerol), phosphatidyl choline, lysophospatidyl choline, phosphatidyl ethanolamine, phosphatidyl serine, phosphatidylinositol, phosphonyl ethanolamine, an ether lipid, monogalactosyl diacylglycerol, digalactosyl diacylglycerol, sulfoquinovosyl diacylglycerol, sphingosine, phytosphingosine, sphingomyelin, glucosylceramide, diacylglyceryl trimethylhomoserine, ricinoleic acid, prostaglandin, jasmonic acid, a-Carotene, b-Carotene, b-cryptoxanthin, astaxanthin, zeaxanthin, chlorophyll a, chlorophyll b, pheophytin a, phylloquinone, plastoquinone, chlorophyllide a, chlorophillide b, pheophorbide a, pyropheophorbide a, pheophorbide b, pheophytin b, hydroxychlorophyll a, hydroxypheophytin a, methoxylactone chlorophyll a, pyrochlorophillide a, pyropheophytin a, diacylglyceryl glucuronide, diacylglyceryl OH methyl carboxy choline, diacylglyceryl OH methyl trimethyl alanine, 2′-O-acyl-sulfoquinovosyldiacylglycerol, phosphatidylinositol-4-phosphate, or phosphatidylinositol-4,5-bisphosphate. In other embodiments, the difference is measured by extraction, gravimetric extraction, or a lipophilic dye. In some embodiments, the extraction is Bligh-Dyer or MTBE. In other embodiments, the difference is an increase or decrease in staining of a cell of the transformed organism using the lipophilic dye. In other embodiments, the lipophilic dye is Bodipy, Nile Red or LipidTOX Green. In one embodiment, the transformed photosynthetic organism is grown in an aqueous environment. In yet another embodiment, the transformed photosynthetic organism is a vascular plant. In another embodiment, the transformed photosynthetic organism is a non-vascular photosynthetic organism. In other embodiments, the transformed photosynthetic organism is an alga or a bacterium. In one embodiment, the bacterium is a cyanobacterium. In other embodiments, the cyanobacterium is a Synechococcus sp., Synechocystis sp., Athrospira sp., Gleocapsa sp., Spinrulina sp., Leptolyngbya sp., Lyngbya sp., Oscillatoria sp., or Pseudoanabaena sp. In another embodiment, the alga is a microalga. In some embodiments, the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. In other embodiments, the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. In yet another embodiment, the C. reinhardtii is wild-type strain CC-1690 21 gr mt+. In one embodiment, the transformed photosynthetic organism's nuclear genome is transformed. In another embodiment, the transformed photosynthetic organism's chloroplast genome is transformed. Yet in another embodiment, the transformed photosynthetic organism's chloroplast genome is transformed and the transformed photosynthetic organism is homoplasmic.

Provided is a method of comparing a first organism's lipid content or profile with a second organism's lipid content or profile, comprising: (a) transforming the first organism with a first polynucleotide, wherein the first polynucleotide comprises: (i) a nucleic acid sequence of SEQ ID NO: 131, 65, 77, 83, 89, 95, 101, 107, 113, 119, 125, 137, 143, 149, 155, 161, 167 or 173; (ii) a nucleotide sequence with at least 80%, at least 85%, at least 90%, at least 95%, at least 98%, or at least 99% sequence identity to the nucleic acid sequence of SEQ ID NO: 131, 65, 77, 83, 89, 95, 101, 107, 113, 119, 125, 137, 143, 149, 155, 161, 167 or 173; (iii) a nucleic acid sequence of SEQ ID NO: 130, 64, 76, 82, 88, 94, 100, 106, 112, 118, 124, 136, 142, 148, 154, 160, 166, or 172; or (iv) a nucleotide sequence with at least 80%, at least 85%, at least 90%, at least 95%, at least 98%, or at least 99% sequence identity to the nucleic acid sequence of 130, 64, 76, 82, 88, 94, 100, 106, 112, 118, 124, 136, 142, 148, 154, 160, 166, or 172; (b) determining the lipid content or profile of the first organism; (c) determining the lipid content or profile of the second organism; and (d) comparing the lipid content or profile of the first organism with the lipid content or profile of the second organism. In another embodiment, the second organism has been transformed with a second polynucleotide. In one embodiment, the lipid content or profile of the first organism is different from the lipid content or profile of the second organism. In some embodiments, the difference is an increase or decrease of one or more of a heme, a polar lipid, a chlorophyll breakdown product, pheophytin, a digalactosyl diacylglycerol (DGDG), a triacylglycerol, a diacylglycerol, a monoacylglycerol, a sterol, a sterol ester, a wax ester, a tocopherol, a fatty acid, phosphatidic acid, lysophosphatidic acid, phosphatidyl glycerol, cardiolipin (diphosphatidylglycerol), phosphatidyl choline, lysophospatidyl choline, phosphatidyl ethanolamine, phosphatidyl serine, phosphatidylinositol, phosphonyl ethanolamine, an ether lipid, monogalactosyl diacylglycerol, digalactosyl diacylglycerol, sulfoquinovosyl diacylglycerol, sphingosine, phytosphingosine, sphingomyelin, glucosylceramide, diacylglyceryl trimethylhomoserine, ricinoleic acid, prostaglandin, jasmonic acid, a-Carotene, b-Carotene, b-cryptoxanthin, astaxanthin, zeaxanthin, chlorophyll a, chlorophyll b, pheophytin a, phylloquinone, plastoquinone, chlorophyllide a, chlorophillide b, pheophorbide a, pyropheophorbide a, pheophorbide b, pheophytin b, hydroxychlorophyll a, hydroxypheophytin a, methoxylactone chlorophyll a, pyrochlorophillide a, pyropheophytin a, diacylglyceryl glucuronide, diacylglyceryl OH methyl carboxy choline, diacylglyceryl OH methyl trimethyl alanine, 2′-O-acyl-sulfoquinovosyldiacylglycerol, phosphatidylinositol-4-phosphate, or phosphatidylinositol-4,5-bisphosphate. In other embodiments, the difference is measured by extraction, gravimetric extraction, or a lipophilic dye. In some embodiments, the extraction is Bligh-Dyer or MTBE. In other embodiments, the difference is an increase or decrease in staining of a cell of the first organism as compared to staining of a cell of the second organism using the lipophilic dye. In yet other embodiments, the lipophilic dye is Bodipy, Nile Red or LipidTOX Green. In one embodiment, the first and second organisms are grown in an aqueous environment. In another embodiment, the first and second organisms are a vascular plant. In yet another embodiment, the first and second organisms are a non-vascular photosynthetic organism. In other embodiments, the first and second organisms are an alga or a bacterium. In one embodiment, the bacterium is a cyanobacterium. In another embodiment, the alga is a microalga. In some embodiments, the microalga is at least one of a Chlamydomonas sp., Volvacales sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. In other embodiments, the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. In one embodiment, the C. reinhardtii is wild-type strain CC-1690 21 gr mt+. In other embodiments, the first and/or second organism's nuclear genome is transformed. In yet other embodiments, the first and/or second organism's chloroplast genome is transformed.

Also provided is a method of increasing production of a lipid, comprising: i) transforming an organism with a polynucleotide comprising a nucleotide sequence encoding a protein that when expressed in the organism results in the increased production of the lipid as compared to an untransformed organism or a second transformed organism, and wherein the nucleotide sequence comprises: (a) a nucleic acid sequence of SEQ ID NO: 131, 65, 77, 83, 89, 95, 101, 107, 113, 119, 125, 137, 143, 149, 155, 161, 167 or 173; (b) a nucleotide sequence with at least 80%, at least 85%, at least 90%, at least 95%, at least 98%, or at least 99% sequence identity to the nucleic acid sequence of SEQ ID NO: 131, 65, 77, 83, 89, 95, 101, 107, 113, 119, 125, 137, 143, 149, 155, 161, 167 or 173; (c) a nucleic acid sequence of SEQ ID NO: 130, 64, 76, 82, 88, 94, 100, 106, 112, 118, 124, 136, 142, 148, 154, 160, 166, or 172; or (d) a nucleotide sequence with at least 80%, at least 85%, at least 90%, at least 95%, at least 98%, or at least 99% sequence identity to the nucleic acid sequence of 130, 64, 76, 82, 88, 94, 100, 106, 112, 118, 124, 136, 142, 148, 154, 160, 166, or 172. In some embodiments, the lipid is stored in a lipid body, a cell membrane, an inter-thylakoid space, and/or a plastoglubuli of the transformed organism. In other embodiments, the method further comprises collecting the lipid from the lipid body of the transformed organism or from the cell membrane of the transformed organism. In some embodiments, the lipid, is any one or more of a heme, a polar lipid, a chlorophyll breakdown product, pheophytin, a digalactosyl diacylglycerol (DGDG), a triacylglycerol, a diacylglycerol, a monoacylglycerol, a sterol, a sterol ester, a wax ester, a tocopherol, a fatty acid, phosphatidic acid, lysophosphatidic acid, phosphatidyl glycerol, cardiolipin (diphosphatidylglycerol), phosphatidyl choline, lysophospatidyl choline, phosphatidyl ethanolamine, phosphatidyl serine, phosphatidylinositol, phosphonyl ethanolamine, an ether lipid, monogalactosyl diacylglycerol, digalactosyl diacylglycerol, sulfoquinovosyl diacylglycerol, sphingosine, phytosphingosine, sphingomyelin, glucosylceramide, diacylglyceryl trimethylhomoserine, ricinoleic acid, prostaglandin, jasmonic acid, a-Carotene, b-Carotene, b-cryptoxanthin, astaxanthin, zeaxanthin, chlorophyll a, chlorophyll b, pheophytin a, phylloquinone, plastoquinone, chlorophyllide a, chlorophillide b, pheophorbide a, pyropheophorbide a, pheophorbide b, pheophytin b, hydroxychlorophyll a, hydroxypheophytin a, methoxylactone chlorophyll a, pyrochlorophillide a, pyropheophytin a, diacylglyceryl glucuronide, diacylglyceryl OH methyl carboxy choline, diacylglyceryl OH methyl trimethyl alanine, 2′-O-acyl-sulfoquinovosyldiacylglycerol, phosphatidylinositol-4-phosphate, or phosphatidylinositol-4,5-bisphosphate. In one embodiment, the transformed organism is grown in an aqueous environment. In another embodiment, the transformed organism is a vascular plant. In another embodiment, the transformed organism is a non-vascular photosynthetic organism. In some embodiments, the transformed organism is an alga or a bacterium. In one embodiment, the bacterium is a cyanobacterium. In other embodiments, the cyanobacterium is a Synechococcus sp., Synechocystis sp., Athrospira sp., Gleocapsa sp., Spirulina sp., Leptolyngbya sp., Lyngbya sp., Oscillatoria sp., or Pseudoanabaena sp. In another embodiment, the alga is a microalga. In some embodiments, the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. In other embodiments, the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. In one embodiment, the C. reinhardtii is wild-type strain CC-1690 21 gr mt+. In one embodiment, the transformed photosynthetic organism's nuclear genome is transformed. In another embodiment, the transformed photosynthetic organism's chloroplast genome is transformed. Yet in another embodiment, the transformed photosynthetic organism's chloroplast genome is transformed and the transformed photosynthetic organism is homoplasmic.

Also provided herein is a method of screening for a protein involved in lipid metabolism in an organism comprising: (a) transforming the organism with a polynucleotide comprising: (i) a nucleic acid sequence of SEQ ID NO: 131, 65, 77, 83, 89, 95, 101, 107, 113, 119, 125, 137, 143, 149, 155, 161, 167 or 173; (ii) a nucleotide sequence with at least 80%, at least 85%, at least 90%, at least 95%, at least 98%, or at least 99% sequence identity to the nucleic acid sequence of SEQ ID NO: 131, 65, 77, 83, 89, 95, 101, 107, 113, 119, 125, 137, 143, 149, 155, 161, 167 or 173; (iii) a nucleic acid sequence of SEQ ID NO: 130, 64, 76, 82, 88, 94, 100, 106, 112, 118, 124, 136, 142, 148, 154, 160, 166, or 172: or (iv) a nucleotide sequence with at least 80%, at least 85%, at least 90%, at least 95%, at least 98%, or at least 99% sequence identity to the nucleic acid sequence of 130, 64, 76, 82, 88, 94, 100, 106, 112, 118, 124, 136, 142, 148, 154, 160, 166, or 172; wherein the transformation of the organism results in expression of a polypeptide encoded by the nucleic acid sequence or nucleotide sequence; and (b) observing a change in expression of an RNA in the transformed organism as compared to an untransformed organism. In one embodiment, the change is an increase in expression of the RNA in the transformed organism as compared to the untransformed organism. In other embodiments, the change is a decrease in expression of the RNA in the transformed organism as compared to the untransformed organism. In other embodiments, the change is measured by microarray, RNA-Seq, or serial analysis of gene expression (SAGE). In other embodiments, the change in expression of an RNA is at least two fold or at least four fold as compared to the untransformed organism. In yet other embodiments, the transformed organism is grown in the presence or absence of nitrogen.

Also provided herein is a higher plant transformed with an isolated polynucleotide comprising: (a) a nucleic acid sequence of SEQ ID NO: 113, 65, 77, 83, 89, 95, 101, 107, 131, 119, 125, 137, 143, 149, 155, 161, 167 or 173: (b) a nucleotide sequence with at least 80%, at least 85%, at least 90%, at least 95%, at least 98%, or at least 99% sequence identity to the nucleic acid sequence of SEQ ID NO: 113, 65, 77, 83, 89, 95, 101, 107, 131, 119, 125, 137, 143, 149, 155, 161, 167 or 173; (c) a nucleic acid sequence of SEQ ID NO: 112, 64, 76, 82, 88, 94, 100, 106, 130, 118, 124, 136, 142, 148, 154, 160, 166, or 172: or (d) a nucleotide sequence with at least 80%, at least 85%, at least 90%, at least 95%, at least 98%, or at least 99% sequence identity to the nucleic acid sequence of 112, 64, 76, 82, 88, 94, 100, 106, 130, 118, 124, 136, 142, 148, 154, 160, 166, or 172; wherein the transformed plant's lipid content or profile is different than an untransformed plant's lipid content or profile or a second transformed plant's lipid content or profile. In some embodiments, the difference is measured by extraction, gravimetric extraction, or a lipophilic dye. In other embodiments, the extraction is Bligh-Dyer or MTBE. In yet other embodiments, the difference is an increase or decrease in staining of a cell of the transformed organism using the lipophilic dye. In other embodiments, the lipophilic dye is Bodipy, Nile Red or LipidTOX Green. In yet other embodiments, the higher plant is Arabidopsis thaliana or a Brassica, Glycine, Gossypium, Medicago, Zea, Sorghum, Oryza, Triticum, or Panicum species.

Provided herein is an isolated polynucleotide, comprising: (a) a nucleic acid sequence of SEQ ID NO: 269, 179, 185, 191, 197, 203, 209, 215, 221, 227, 233, 239, 245, 251, 257, 263, 275, 281, 287, 293, or 299; (b) a nucleotide sequence with at least 80%, at least 85%, at least 90%, at least 95%, at least 98%, or at least 99% sequence identity to the nucleic acid sequence of SEQ ID NO: 269, 179, 185, 191, 197, 203, 209, 215, 221, 227, 233, 239, 245, 251, 257, 263, 275, 281, 287, 293, or 299; (c) a nucleic acid sequence of SEQ ID NO: 268, 178, 184, 190, 196, 202, 208, 214, 220, 226, 232, 238, 244, 250, 256, 262, 274, 280, 286, 292, or 298; or (d) a nucleotide sequence with at least 80%, at least 85%, at least 90%, at least 95%, at least 98%, or at least 99% sequence identity to the nucleic acid sequence of 268, 178, 184, 190, 196, 202, 208, 214, 220, 226, 232, 238, 244, 250, 256, 262, 274, 280, 286, 292, or 298. Also provided herein is organism transformed with the isolated polynucleotide and a vector comprising the isolated polynucleotide. In one embodiment, the vector further comprises a 5′ regulatory region. In another embodiments, the 5′ regulatory region further comprises a promoter. The promoter may be a constitutive promoter or an inducible promoter. In some embodiments, the inducible promoter is a light inducible promoter, a nitrate inducible promoter, or a heat responsive promoter. In another embodiment, the vector further comprises a 3′ regulatory region.

Also provided is an isolated polynucleotide encoding a protein comprising, (a) an amino acid sequence of SEQ ID NO: 270, 180, 186, 192, 198, 204, 210, 216, 222, 228, 234, 240, 246, 252, 258, 264, 276, 282, 288, 294, or 300; or (b) a homolog of the amino acid sequence of (a), wherein the homolog has at least 80%, at least 85%, at least 90%, at least 95%, at least 98%, or at least 99% sequence identity to the amino acid sequence of SEQ ID NO: 270, 180, 186, 192, 198, 204, 210, 216, 222, 228, 234, 240, 246, 252, 258, 264, 276, 282, 288, 294, or 300. Also provided is an organism transformed with the isolated polynucleotide wherein the protein encoded by the polynucleotide is expressed.

Provided herein is a photosynthetic organism transformed with an isolated polynucleotide comprising: (a) a nucleic acid sequence of SEQ ID NO: 269, 179, 185, 191, 197, 203, 209, 215, 221, 227, 233, 239, 245, 251, 257, 263, 275, 281, 287, 293, or 299; (b) a nucleotide sequence with at least 80%, at least 85%, at least 90%, at least 95%, at least 98%, or at least 99% sequence identity to the nucleic acid sequence of SEQ ID NO: 269, 179, 185, 191, 197, 203, 209, 215, 221, 227, 233, 239, 245, 251, 257, 263, 275, 281, 287, 293, or 299; (c) a nucleic acid sequence of SEQ ID NO: 268, 178, 184, 190, 196, 202, 208, 214, 220, 226, 232, 238, 244, 250, 256, 262, 274, 280, 286, 292, or 298; or (d) a nucleotide sequence with at least 80%, at least 85%, at least 90%, at least 95%, at least 98%, or at least 99% sequence identity to the nucleic acid sequence of 268, 178, 184, 190, 196, 202, 208, 214, 220, 226, 232, 238, 244, 250, 256, 262, 274, 280, 286, 292, or 298; wherein the transformed organism's growth is increased as compared to an untransformed organism's growth or a second transformed organism's growth. In one embodiment, the increase in growth is determined by a competition assay between at least the transformed organism and the untransformed organism. In another embodiment, the competition assay comprises an additional organism. In another embodiment, the competition assay is in one or more turbidostats. In some embodiments, the transformed organism's increase in growth is measured by growth rate, carrying capacity, or culture productivity. In other embodiments, the transformed organism has at least a 2%, at least a 4%, at least a 6%, at least a 8%, at least a 10%, at least a 12%, at least a 14%, at least a 16%, at least a 18%, at least a 20%, at least a 22%, at least a 24%, at least a 26%, at least a 28%, at least a 30%, at least a 50%, at least a 100%, at least a 150%, at least a 200%, at least a 250%, at least a 300%, at least a 350%, or at least a 400% increase in growth rate as compared to either the untransformed organism or the second transformed organism. In yet other embodiments, the transformed organism has from a 0.01% to a 2.0%, from a 2% to a 4%, from a 4% to a 6%, from a 6% to a 8%, from a 8% to a 10%, from a 10% to a 12%, from a 12% to a 14%, from a 14% to a 16%, from a 16% to a 18%, from a 18% to a 20%, from a 20% to a 22%, from a 22% to a 24%, from a 24% to a 26%, from a 26% to a 28%, from a 28% to a 30%, from a 30% to a 50%, from a 50% to a 100%, from a 100% to a 150%, from a 150% to a 200%, from a 200% to a 250%, from a 250% to a 300%, from a 300% to a 350%, from a 3500% to a 400%, or a 400% to a 600% increase in growth rate as compared to either the untransformed organism or the second transformed organism. In one embodiment, the increase is shown by the transformed organism having a positive selection coefficient as compared to either the untransformed organism or the second transformed organism. In another embodiment, the transformed organism is grown in an aqueous environment. In one embodiment, the transformed organism is a vascular plant. In another embodiment, the transformed organism is a non-vascular photosynthetic organism. In some embodiments, the transformed organism is an alga or a bacterium. In one embodiment, the bacterium is a cyanobacterium. In other embodiments, the cyanobacterium is a Synechococcus sp., Synechocystis sp., Athrospira sp., Gleocapsa sp., Spirulina sp., Leptolyngbya sp., Lyngbya sp., Oscillatoria sp., or Pseudoanabaena sp. In another embodiment, the alga is a microalga. In other embodiments, the microalga is at least one of a Chlamydomonas sp., Volvacales sp., Desmid sp., Dunaliella sp., Scenedesmus sp., Chlorella sp., Hemataoccus sp., Volvox sp., Nannochloropsis sp., Arthrospira sp., Sprirulina sp., Botryococcus sp., Haematococcus sp., or Desmodesmus sp. In yet other embodiments, the microalga is at least one of Chlamydononar reinhardii, N. oceanica, N. salina, Dunaliella salina, H. plurvalis, S. dimorphus, Dunaliella viridis, N. oculata, Dunaliella ertioleclta, S. Maximus, or A. Fusiformus. In one embodiment, the C. reinhardtii is wild-type strain CC-1690 21 gr mt+.

Also provided herein is a method of comparing the growth of a first organism with a growth of a second organism, comprising: (a) transforming the first organism with a first polynucleotide, wherein the first polynucleotide comprises: (i) a nucleic acid sequence of SEQ ID NO: 269, 179, 185, 191, 197, 203, 209, 215, 221, 227, 233, 239, 245, 251, 257, 263, 275, 281, 287, 293, or 299: (ii) a nucleotide sequence with at least 80%, at least 85%, at least 90%, at least 95%, at least 98%, or at least 99% sequence identity to the nucleic acid sequence of SEQ ID NO: 269, 179, 185, 191, 197, 203, 209, 215, 221, 227, 233, 239, 245, 251, 257, 263, 275, 281, 287, 293, or 299: (iii) a nucleic acid sequence of SEQ ID NO: 268, 178, 184, 190, 196, 202, 208, 214, 220, 226, 232, 238, 244, 250, 256, 262, 274, 280, 286, 292, or 298; or (iv) a nucleotide sequence with at least 80%, at least 85%, at least 90%, at least 95%, at least 98%, or at least 99% sequence identity to the nucleic acid sequence of 268, 178, 184, 190, 196, 202, 208, 214, 220, 226, 232, 238, 244, 250, 256, 262, 274, 280, 286, 292, or 298; (b) measuring the growth of the first organism; (c) measuring the growth of the second organism: and (d) comparing the growth of the first organism with the growth of the second organism. In one embodiment, the second organism has been transformed with a second polynucleotide. In another embodiment, the growth of the first organism is increased as compared to the growth of the second organism. In another embodiment, the growth is determined by a competition assay between at least the first transformed organism and the second organism. In yet another embodiment, the competition assay comprises an additional organism. In one embodiment, the competition assay is in one or more turbidostats. In other embodiments, the first organism's growth and the second organism's growth is measured by growth rate, carrying capacity, or culture productivity. In other embodiments, the first transformed organism has at least a 2%, at least a 4%, at least a 6%, at least a 8%, at least a 10%, at least a 12%, at least a 14%, at least a 16%, at least a 18%, at least a 20%, at least a 22%, at least a 24%, at least a 26%, at least a 28%, at least a 30%, at least a 50%, at least a 100%, at least a 150%, at least a 200%, at least a 250%, at least a 300%, at least a 350%, or at least a 400% increase in growth rate as compared to the second organism. In another embodiment, the first transformed organism has a positive selection coefficient as compared to the second organism. In one embodiment, the organism is grown in an aqueous environment. The organism may be a vascular plant or a non-vascular photosynthetic organism. The organism may be an alga or a bacterium. In one embodiment, the bacterium is a cyanobacterium. In another embodiment, the alga is a microalga. In some embodiments, the microalga is at least one of a Chlamydomonas sp., Volvacales sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp. In other embodiments, the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus. In one embodiment, the C. reinhardtii is wild-type strain CC-1690 21 gr mt+. In one embodiment, the first and or second organism's nuclear genome is transformed. In another embodiment, the first and or second organism's chloroplast genome is transformed.

Also provided is a method of screening for a protein involved in growth of an organism comprising: (a) transforming the organism with a polynucleotide comprising: (i) a nucleic acid sequence of SEQ ID NO: 269, 179, 185, 191, 197, 203, 209, 215, 221, 227, 233, 239, 245, 251, 257, 263, 275, 281, 287, 293, or 299: (ii) a nucleotide sequence with at least 80%, at least 85%, at least 90%, at least 95%, at least 98%, or at least 99% sequence identity to the nucleic acid sequence of SEQ ID NO: 269, 179, 185, 191, 197, 203, 209, 215, 221, 227, 233, 239, 245, 251, 257, 263, 275, 281, 287, 293, or 299; (iii) a nucleic acid sequence of SEQ ID NO: 268, 178, 184, 190, 196, 202, 208, 214, 220, 226, 232, 238, 244, 250, 256, 262, 274, 280, 286, 292, or 298; or (iv) a nucleotide sequence with at least 80%, at least 85%, at least 90%, at least 95%, at least 98%, or at least 99% sequence identity to the nucleic acid sequence of 268, 178, 184, 190, 196, 202, 208, 214, 220, 226, 232, 238, 244, 250, 256, 262, 274, 280, 286, 292, or 298; wherein the transformation of the organism results in expression of a polypeptide encoded by the nucleic acid sequence or nucleotide sequence; and (b) observing a change in expression of an RNA in the transformed organism as compared to an untransformed organism. In one embodiment, the change is an increase in expression of the RNA in the transformed organism as compared to the untransformed organism. In another embodiment, the change is a decrease in expression of the RNA in the transformed organism as compared to the untransformed organism. In other embodiments, the change is measured by microarray, RNA-Seq, or serial analysis of gene expression (SAGE). In still other embodiments, the change is at least two fold or at least four fold as compared to the untransformed organism. In one embodiment, the transformed organism is grown in the absence of nitrogen.

Provided herein is a higher plant transformed with an isolated polynucleotide comprising: (a) a nucleic acid sequence of SEQ ID NO: 269, 179, 185, 191, 197, 203, 209, 215, 221, 227, 233, 239, 245, 251, 257, 263, 275, 281, 287, 293, or 299; (b) a nucleotide sequence with at least 80%, at least 85%, at least 90%, at least 95%, at least 98%, or at least 99% sequence identity to the nucleic acid sequence of SEQ ID NO: 269, 179, 185, 191, 197, 203, 209, 215, 221, 227, 233, 239, 245, 251, 257, 263, 275, 281, 287, 293, or 299; (c) a nucleic acid sequence of SEQ ID NO: 268, 178, 184, 190, 196, 202, 208, 214, 220, 226, 232, 238, 244, 250, 256, 262, 274, 280, 286, 292, or 298; or (d) a nucleotide sequence with at least 80%, at least 85%, at least 90%, at least 95%, at least 98%, or at least 99% sequence identity to the nucleic acid sequence of 268, 178, 184, 190, 196, 202, 208, 214, 220, 226, 232, 238, 244, 250, 256, 262, 274, 280, 286, 292, or 298, wherein the transformed organism's growth is increased as compared to an untransformed organism's growth or a second transformed organism's growth. In some embodiments, the increase in growth is measured by a competition assay, growth rate, carrying capacity, culture productivity, cell proliferation, seed yield, organ growth, or polysome accumulation. In one embodiment, the increase is measured by growth rate. In some embodiments, the transformed organism has at least a 2%, at least a 4%, at least a 6%, at least a 8%, at least a 10%, at least a 12%, at least a 14%, at least a 16%, at least a 18%, at least a 20%, at least a 22%, at least a 24%, at least a 26%, at least a 28%, at least a 30%, at least a 50%, at least a 100%, at least a 150%, at least a 200%, at least a 250%, at least a 300%, at least a 350%, or at least a 400% increase in growth rate as compared to the untransformed organism or the second transformed organism. In yet other embodiments, the transformed higher plant has from a 0.01% to a 2.0%, from a 2% to a 4%, from a 4% to a 6%, from a 6% to a 8%, from a 8% to a 10%, from a 10% to a 12%, from a 12% to a 14%, from a 14% to a 16%, from a 16% to a 18%, from a 18% to a 20%, from a 20% to a 22%, from a 22% to a 24%, from a 24% to a 26%, from a 26% to a 28%, from a 28% to a 30%, from a 30% to a 50%, from a 50% to a 100%, from a 100% to a 150%, from a 150% to a 200%, from a 200% to a 250%, from a 250% to a 300%, from a 300% to a 350%, from a 350% to a 400%, or a 400% to a 600% increase in growth rate as compared to either the untransformed plant or the second transformed plant. In one embodiment, the higher plant is Arabidopsis thaliana. In some embodiments, the higher plant is a Brassica, Glycine, Gossypium, Medicago, Zea, Sorghum, Orjza, Triticum, or Panicum species.

### BRIEF DESCRIPTION OF THE DRAWINGS

These and other features, aspects, and advantages of the present disclosure will become better understood with regard to the following description, appended claims and accompanying figures.

FIG. 1 shows cellular lipid content in various classes of microalgae and cyanobacteria under normal growth (NG) and stress conditions (SC). (a) green microalgae; (b) diatoms; (c) oleaginous species/strains from other eukaryotic algal taxa; and (d) cyanobacteria. Open circles: cellular lipid contents obtained under normal growth or nitrogen-replete conditions. Closed circles: cellular lipid contents obtained under nitrogen-depleted or other stress conditions. The differences in cellular lipid content between cultures under normal growth and stress growth conditions were statistically significant for all three groups (a, b and c) of algae examined using Duncan's multiple range test with the ANOVA procedure.

FIG. 2 shows fatty acid de novo synthesis pathway in chloroplasts. Acetyl CoA enters the pathway as a substrate for acetyl CoA carboxylase (Reaction 1) as well as a substrate for the initial condensation reaction (Reaction 3). Reaction 2, which is catalyzed by malonyl CoA:ACP transferase and transfers malonyl from CoA to form malonyl ACP. Malonyl ACP is the carbon donor for subsequent elongation reactions. After subsequent condensations, the 3-ketoacyl ACP product is reduced (Reaction 4), dehydrated (Reaction 5) and reduced again (Reaction 6), by 3-ketoacyl ACP reductase, 3-hydroxyacyl ACP dehydrase and enoyl ACP reductase, respectively (adapted and modified from Ohlrogge and Browse, 1995, Plant Cell, 7, 957-970).

FIG. 3 is a simplified schematic showing the triacylglycerol (TAG) biosynthesis pathway in algae. (1) Cytosolic glycerol-3-phosphate acyl transferase, (2) lyso-phosphatidic acid acyl transferase, (3) phosphatidic acid phosphatase, and (4) diacylglycerol acyl transferase. Adapted from Roessler et al., 1994, Genetic engineering approaches for enhanced production of biodiesel fuel from microalgae. In Enzymatic Conversion of Biomass for Fuels Production (Himmel, M. E., Baker, J. and Overend, R. P., eds). American Chemical Society, pp. 256-270.

FIG. 4 shows fermentative pathways identified in Chlamydomonas reinhardii following anaerobic incubation (adapted and modified from Mus et al., 2007, J. Biol. Chem. 282, 25475-25486). Under aerobic conditions, pyruvate is metabolized predominantly by the pyruvate dehydrogenase complex to produce NADH and acetyl CoA, the latter of which ties into lipid metabolism (see FIG. 5). ACK, acetate kinase; ADH, alcohol dehydrogenase: ADHE, alcohol aldehyde bifunctional dehydrogenase; H2ase, hydrogenase; PAT, phosphotransacetylase; PDC, pyruvate decarboxylase; PFL, pyruvate formate lyase; PFR, pyruvate ferredoxin oxidoreductase.

FIG. 5 shows pathways of lipid biosynthesis that are known or hypothesized to occur in Chlamydomonas, and their presumed subcellular localizations. Abbreviations: ACP, acyl carrier protein; AdoMet, S-adenosylmethionine; ASQD, 2′-O-acyl sulfoquinovosyldiacylglycerol; CDP, cytidine-5′-diphosphate; CoA, coenzyme A; CTP, cytidine-5′-triphosphate; DAG, diacylglycerol; DGDG, digalactosyldiacylglycerol; DGTS, diacylglyceryl N,N,N-trimethylhomoserine; Etn, ethanolamine: FA, fatty acid; G-3-P, glycerol-3-phosphate; Gic, glucose; Glc-1-P, glucose-1-phosphate; Ins, inositol; Ins-3-P, inositol-3-phosphate; Met, methionine; MGDG, mono-galactosyldiacylglycerol; P-Etnm, phosphoethanolamine; PtdEtn, phosphatidylethanolamine; PtdGro, phosphatidylglycerol; PtdGroP, phosphatidylglycerophosphate; PtdIns, phosphatidylinositol; PtdOH, phosphatidic acid; Ser, serine; SQ, sulfoquinovose; SQDG, sulfoquinovosyldiacylglycerol; UDP, uridine-5-diphosphate (as described in Riekhof, W. R., et al., 2005, Eukaryotic Cell. 4, 242-252).

FIG. 6 shows an exemplary expression vector (SEnuc357) that can be used with the embodiments disclosed herein.

FIG. 7 shows an exemplary expression vector that can be used with the embodiments disclosed herein.

FIGS. 8A, 8B, 8C, and 8D show typical nitrogen stress phenotypes.

FIG. 8A shows percent lipid levels in three algal strains (SE0004 is Scenedesmus dimorphus; SE0043 is Dunaliella Salina; and SE0050 is Chlamydomonas reinhardtii) in the presence and absence of nitrogen.

FIG. 8B shows percent lipid levels in the two algal strains shown in FIG. 8A with the addition of SE0003 (Dunaliella salina).

FIG. 8C shows growth of Chlamydomonas reinhardtii in the presence and absence of nitrogen.

FIG. 8D shows chlorophyll levels in Chlamydomonas reinhardtii in the presence and absence of nitrogen over a 9-day time course.

FIG. 9 shows total fat analysis via HPLC-CAD in the presence and absence of nitrogen (24 hour time point). No significant difference was observed in the two spectra after 24 hours in the absence of nitrogen.

FIG. 10 shows total fat analysis via HPLC-CAD in the presence and absence of nitrogen (48 hour time point). There is an increase in neutral lipid (*) peaks (44 to 54 minute retention time) after 48 hours in the absence of nitrogen.

FIG. 11 shows up regulation of genes by qPCR in Chlamydomonas reinhardtii grown in TAP (Tris-acetate-phosphate) in the absence of nitrogen (24 hour time point).

FIG. 12 shows down regulation of genes by qPCR in Chlamydomonas reinhardtii grown in TAP in the absence of nitrogen (24 hour time point).

FIG. 13 describes the RNA-Seq transcriptomic method.

FIG. 14 shows all Chlamydomonas reinhardtii genes and their expression levels at a six hour time point generated by the method described in FIG. 13 in the presence and absence of nitrogen. White dots represent genes that are up or down regulated at least four fold at the six hour time point.

FIG. 15 shows gene expression levels across a time course of nitrogen starvation (as described in Table 2). Each line represents a different gene.

FIG. 16 shows the expression levels of the 14 target genes that were selected. Gene expression levels are across a time course of nitrogen starvation (as described in Table 2). Each line represents a different gene.

FIG. 17 shows a cloning vector used for cloning SN (stress-nitrogen) targets into algae.

FIG. 18 describes the distribution of Chlamydomonas reinhardii strains overexpressing SN01, SN02, and SN03 after FACS enrichment for high-lipid dye staining.

FIGS. 19A, 19B, 19C, and 19D show flow cytometry (Guava) results for SN03 strains identified from the FACS experiment of FIG. 18. FIGS. 19A and B use Bodipy dye; FIG. 19C uses Lipid TOX green; and FIG. 19D uses Nile Red. Wild type is Chlamydomonas reinhardtii replicates and the numbers represent the various SN03 strains.

FIGS. 20A and 20B show Chlamydomonas reinhardtii strains overexpressing SN03 grown on TAP or high salt media (HSM) and then MTBE extracted for lipid content.

FIG. 21 shows 1D 1H NMR of the MTBE extracted oil from wild type Chlamydomonas reinhardii grown in the presence and absence of nitrogen and a Chlamydomonas reinhardtii strain overexpressing SN03 (SN03-34).

FIGS. 22A and B shows close to of peaks from the experiment described in FIG. 21.

FIGS. 23A, 23B, and 23C show the growth rates of Chlamydomonas reinhardtii strains overexpressing SN03. Gene negative is a control Chlamydomonas reinhardtii transgenic line in which the SN03 open reading frame was truncated. Wild type is Chlamydomonas reinhardtii. FIGS. 23A and B represent strains grown in TAP and FIG. 23C represents strains grown in HSM.

FIG. 24 shows SN03 RNA levels by qPCR in Chlamydomonas reinhardtii strains overexpressing SN03.

FIG. 25 shows SN03 protein expression levels in Chlamydomonas reinhardtii strains overexpressing SN03.

FIG. 26 shows a reference trace for hexane extracted total lipid for Chlamydomonas reinhardtii using HPLC and a charged Aerosol detector (CAD).

FIG. 27 shows HPLC data from MTBE extracted oil from Chlamydomonas reinhardtii strains overexpressing SN03 and MTBE extracted oil from wild type Chlamydomonas reinhardtii grown in the presence and absence of nitrogen.

FIG. 28 shows Flow cytometry results of Chlamydomonas reinhardtii strains overexpressing SN03 confirming a high lipid phenotype using several different lipid dyes. The left hand column of each group represents staining with Bodipy. The middle column of each group represents staining with Nile Red. The right hand column of each group represents staining with LipidTOX Green. Wild type is Chlamydomonas reinhardtii replicates and SN03-2, -3, -15, -32, and -34 represent the various SN03 strains.

FIG. 29 shows Chlamydomonas reinhardtii strains overexpressing SN03 grown on TAP and MTBE extracted for lipid content.

FIG. 30 shows chlorophyll levels in Chlamydomonas reinhardtii wild type and Chlamydomonas reinhardtii strains overexpressing SN03 in the presence and absence of nitrogen.

FIG. 31 shows growth rates of Chlamydomonas reinhardtii wild type and Chlamydomonas reinhardtii strains overexpressing SN03.

FIG. 32 shows induction of endogenous SN03 and stress-induced protein kinase (PK) upon nitrogen starvation in Chlamydomonas reinhardtii wild type and Chlamydomonas reinhardtii expressing a miRNA specific to SN03 (knock-down). The left hand column of each group represents a stressed induced PK and the right hand column of each group represents endogenous SN03 (147817). The x-axis represents the various knock-down lines.

FIG. 33 shows MTBE extraction of wild type Chlamydomonas reinhardtii and a Chlamydomonas reinhardtii strain expressing a miRNA specific to SN03 (knock-down). The two strains are grown in the presence and absence of nitrogen. The knock-down strain demonstrates that SN03 is necessary for lipid accumulation upon nitrogen starvation.

FIG. 34 shows a cloning vector (Ble2A-SN03) used for cloning SN (stress-nitrogen) targets into algae. The vector used the AR4 promoter to drive a bleomycin resistance gene and the SN gene. It has an ampicillin resistance cassette for growth in bacteria.

FIG. 35 shows an exemplary expression vector (SEnuc357_SN03) that can be used with the embodiments disclosed herein.

FIG. 36 shows all Chlamydomonas reinhardtii genes and their expression levels at a six hour time point generated by the method described in FIG. 13 in the presence and absence of nitrogen. White dots represent genes that are up regulated four fold or greater in a Chlamydomonas reinhardii strain overexpressing SN03.

FIG. 37 shows all Chlamydomonas reinhardtii genes and their expression levels at a six hour time point generated by the method described in FIG. 13 in the presence and absence of nitrogen. White dots represent genes that are down regulated four fold or greater in a Chlamydomonas reinhardtii strain overexpressing SN03.

FIG. 38 shows expression levels of endogenous and transgenic SN03 RNA in wild type Chlamydomonas reinhardtii over a time course of nitrogen starvation and expression levels of endogenous and transgenic SN03 RNA in SN03 overexpressing strains. Transgenic (Ble) SN03 is represented by the continuous line and endogenous SN03 is represented by the broken line.

FIG. 39 shows expression levels of endogenous and transgenic SN03 RNA in wild type Chlamydomonas reinhardtii over a time course of nitrogen starvation and expression levels of endogenous and transgenic SN03 RNA in SN03 overexpressing strains. The left hand column of each pair represents Transgenic (Ble) SN03 and the right hand column of each pair represents endogenous SN03.

FIG. 40 shows gene expression levels in wild type Chlamydomonas reinhardtii over a time course of nitrogen starvation and gene expression levels in SN03 overexpressing strains. Each line represents a different gene. The genes shown are upregulated in nitrogen starvation and down regulated in SN03 overexpressing strains.

FIG. 41A shows growth of wild-type Nannochloropsis salina in modified artificial sea water media (MASM) media in the presence and absence of nitrogen. The diamonds represent growth in the presence of nitrogen and squares represent growth in the absence of nitrogen.

FIG. 41B shows chlorophyll levels of wild-type Nannochloropsis salina in modified artificial sea water media (MASM) media in the presence and absence of nitrogen.

FIG. 41C shows MTBE extraction of wild-type Nannochloropsis salina in MASM media in the presence and absence of nitrogen.

FIG. 41D shows growth of wild-type Scenedesmus dimorphus in HSM media in the presence and absence of nitrogen. The diamonds represent growth in the presence of nitrogen and squares represent growth in the absence of nitrogen.

FIG. 41E shows chlorophyll levels of wild-type Scenedesmus dimorphus in HSM media in the presence and absence of nitrogen.

FIG. 42A shows the distribution of Chlamydomonas reinhardtii strains overexpressing SN01, SN02, and SN03 after FACS enrichment for high-lipid dye staining. The solid portion of each bar represents the percentage of lines overexpressing SN03; the striped portion of each bar represents the percentage of lines overexpressing SN02, and the unfilled portion of each bar represents the percentage of lines overexpressing SN01.

FIG. 42B shows flow cytometry (Guava) results for wild-type Chlamydomonas reinhardtii in the presence and absence of nitrogen and an SN03 overexpressing strain. The left hand column of each set is Nile Red: the middle column of each set is LipidTOX green; and the right hand column of each set is Bodipy.

FIG. 42C shows flow cytometry (Guava) results using Bodipy for wild-type Chlamydomonas reinhardtii and several SN03 overexpressing strains.

FIG. 43 shows the genomic integration site of the SN03 vector (as shown in FIG. 34) for two SN03 overexpression cell lines.

FIG. 44A shows SN03 protein expression levels in a Chlamydomonas reinhardtii SN03 overexpressing strain. Bacterial alkaline phosphatase (BAP) was used as a positive control.

FIG. 44B shows SN03 RNA levels by qPCR in Chlamydomonas reinhardtii strains overexpressing SN03. Expression of SN03 RNA in wild-type Chlamydomonas reinhardtii was not detected (N.D.).

FIG. 45A shows wild-type Chlamydomonas reinhardtii in the presence and absence of nitrogen and Chlamydomonas reinhardtii strains overexpressing SN03 MTBE extracted for lipid content.

FIG. 45B shows the growth rates of wild-type Chlamydomonas reinhardii and a Chlamydomonas reinhardtii strain overexpressing SN03 in HSM.

FIG. 45C shows the carrying capacity of wild-type Chlamydomonas reinhardii grown in the presence and absence of nitrogen and an SN03 overexpression line grown in the presence and absence of nitrogen.

FIG. 45D shows the chlorophyll levels of wild-type Chlamydomonas reinhardtii grown in the presence and absence of nitrogen and an SN03 overexpression line grown in the presence and absence of nitrogen.

FIG. 46A shows MTBE extraction of wild type Chlamydomonas reinhardtii and three SN03 knockdown lines in the presence and absence of nitrogen.

FIG. 46B shows upregulation of SN03 RNA and a stress induced protein kinase RNA by qPCR in wild type Chlamydomonas reinhardtii and three SN03 knockdown lines upon nitrogen starvation.

FIG. 47A shows flow cytometry (Guava) results using Nile Red for wild-type Chlamydomonas reinhardtii and several SN03 overexpressing strains. “C” represents the codon-optimized endogenous SN03 sequence (SEQ ID NO: 13) from Chlamydomonas reinhardtii with a nucleotide sequence coding for a FLAG-MAT tag at the 3′ end.

FIG. 47B shows flow cytometry (Guava) results using Nile Red for wild-type Chlamydomonas reinhardtii and several SN03 overexpressing strains. “E” represents the endogenous SN03 sequence (SEQ ID NO: 10) from Chlamydomonas reinhardtii with a nucleotide sequence coding for a FLAG-MAT tag at the 3′ end.

FIG. 48 shows wild-type Chlamydomonas reinhardii and Chlamydomonas reinhardtii strains overexpressing SN03 MTBE extracted for lipid content. “C” represents the codon-optimized endogenous SN03 sequence (SEQ ID NO: 13)) from Chlamydomonas reinhardtii with a nucleotide sequence coding for a FLAG-MAT tag at the 3′ end.

FIG. 49 shows a protein alignment of the U.S. Department of Energy (DOE) Joint Genome Institute (JGI) annotated SN03 sequence (SEQ ID NO: 6) and the endogenous SN03 sequence (SEQ ID NO: 14).

FIG. 50 shows the presence of lipid bodies in wild type Chlamydomonas reinhardtii in the absence of nitrogen, and in an SN03 overexpression line. Top left panel is wild type Chlamydomonas reinhardtii in the presence of nitrogen. Top right panel is wild type Chlamydomonas reinhardtii in the absence of nitrogen. Bottom panels are two images of an SN03 overexpression line. The dye used was Nile Red.

FIG. 51 shows HPLC analyses of wild type and SN03 knock-down line in the presence and absence of nitrogen.

FIG. 52 shows a miRNA expression vector.

FIG. 53 shows analytical flow cytometry (Guava) data for the SN01 over expression cell line. The left-hand column of each set of three columns represents cells stained with Bodipy lipid dye; the middle column represents cells stained with Nile Red lipid dye; and the right-hand column represents cells stained with LipidTOX lipid dye. The x-axis shows 12 independent cell lines and the y-axis shows the fold difference in staining relative to the wild type strain.

FIG. 54 shows analytical flow cytometry (Guava) data for the SN08 over expression cell line. The left-hand column of each set of three columns represents cells stained with Bodipy lipid dye; the middle column represents cells stained with Nile Red lipid dye; and the right-hand column represents cells stained with LipidTOX lipid dye. The x-axis shows 12 independent cell lines and the y-axis shows the fold difference in staining relative to the wild type strain.

FIG. 55 shows analytical flow cytometry (Guava) data for the SN87 over expression cell line. The left-hand column of each set of three columns represents cells stained with Bodipy lipid dye; the middle column represents cells stained with Nile Red lipid dye; and the right-hand column represents cells stained with LipidTOX lipid dye. The x-axis shows 12 independent cell lines and the y-axis shows the fold difference in staining relative to the wild type strain.

FIG. 56 shows analytical flow cytometry (Guava) data for the SN120 over expression cell line. The left-hand column of each set of three columns represents cells stained with Bodipy lipid dye; the middle column represents cells stained with Nile Red lipid dye; and the right-hand column represents cells stained with LipidTOX lipid dye. The x-axis shows 12 independent cell lines and the y-axis shows the fold difference in staining relative to the wild type strain.

FIG. 57 shows the growth rate (on the y-axis) for several SN79 transgenic lines along with a wild type Chlamydomonas reinhardtii line (shown along the x-axis).

FIG. 58 shows the growth rate (on the y-axis) for several SN64 transgenic lines along with a wild type Chlamydomonas reinhardtii line (shown along the x-axis).

FIG. 59 shows the growth rate (on the y-axis) for several SN24 transgenic lines along with a wild type Chlamydomonas reinhardtii line (shown along the x-axis).

FIG. 60 shows the growth rate (on the y-axis) for several SN82 transgenic lines along with a wild type Chlamydomonas reinhardtii line (shown along the x-axis).

FIG. 61 shows the growth rate (on the y-axis) for several SN01 transgenic lines along with a wild type Chlamydomonas reinhardtii line (shown along the x-axis).

FIG. 62 shows the growth rate (on the y-axis) for several SN28 transgenic lines along with a wild type Chlamydomonas reinhardtii line (shown along the x-axis).

FIG. 63 shows a vector SENuc745.

FIG. 64 shows a vector SENuc744.

FIG. 65 shows data from a 96-well micro plate growth assay measuring the growth rate (r) of individual SN gene transformants. 5 transformants were analyzed for SN78. The data were analyzed by Oneway ANOVA of r by transformant (line) using Dunnett's test for multiple comparisons with control.

FIG. 66 shows data from a 96-well micro plate growth assay measuring the theoretical peak productivity (Kr/4) of individual SN gene transformants. 8 transformants were analyzed for SN24, 8 transformants were analyzed for SN26, and 10 transformants were analyzed for SN39. The data was analyzed by Oneway ANOVA of Kr/4 by transformant (line) using Dunnett's test for multiple comparisons with control.

FIG. 67 shows a Logistical Model and the First Derivative of the Model Fit as described in Example 21.

FIG. 68 shows analytical flow cytometry (Guava) data for several SN over expression cell lines stained with Bodipy lipid dye analyzed by Oneway ANOVA of individual SN cell lines using Dunnett's test for multiple comparisons with control.

FIG. 69 shows analytical flow cytometry (Guava) data for several SN over expression cell lines stained with Nile Red lipid dye analyzed by Oneway ANOVA of individual SN cell lines using Dunnett's test for multiple comparisons with control.

FIG. 70 shows analytical flow cytometry (Guava) data for several SN over expression cell lines stained with LipidTox lipid dye analysed by Oneway ANOVA of individual SN cell lines using Dunnett's test for multiple comparisons with control.

FIG. 71 shows analytical flow cytometry (Guava) data for several SN over expression cell lines stained with Bodipy lipid dye analysed by Oneway ANOVA of individual SN cell lines using Dunnett's test for multiple comparisons with control.

FIG. 72 shows analytical flow cytometry (Guava) data for several SN over expression cell lines stained with Nile Red lipid dye analysed by Oneway ANOVA of individual SN cell lines using Dunnett's test for multiple comparisons with control.

FIG. 73 shows analytical flow cytometry (Guava) data for several SN over expression cell lines stained with LipidTox lipid dye analysed by Oneway ANOVA of individual SN cell lines using Dunnett's test for multiple comparisons with control.

### DETAILED DESCRIPTION

The following detailed description is provided to aid those skilled in the art in practicing the present disclosure. Even so, this detailed description should not be construed to unduly limit the present disclosure as modifications and variations in the embodiments discussed herein can be made by those of ordinary skill in the art without departing from the spirit or scope of the present disclosure.

As used in this specification and the appended claims, the singular forms “a”, “an” and “the” include plural reference unless the context clearly dictates otherwise.

Endogenous

An endogenous nucleic acid, nucleotide, polypeptide, or protein as described herein is defined in relationship to the host organism. An endogenous nucleic acid, nucleotide, polypeptide, or protein is one that naturally occurs in the host organism.

Exogenous

An exogenous nucleic acid, nucleotide, polypeptide, or protein as described herein is defined in relationship to the host organism. An exogenous nucleic acid, nucleotide, polypeptide, or protein is one that does not naturally occur in the host organism or is a different location in the host organism.

Nucleic Acid and Protein Sequences

The following nucleic acid and amino acid sequences are useful in the disclosed embodiments.

If an initial start codon (Met) is not present in any of the amino acid sequences disclosed herein, including sequences contained in the sequence listing, one of skill in the art would be able to include, at the nucleotide level, an initial ATG, so that the translated polypeptide would have the initial Met. If a start and/or stop codon is not present at the beginning and/or end of a coding sequence, one of skill in the art would know to insert an “ATG” at the beginning of the coding sequence and nucleotides encoding for a stop codon (any one of TAA, TAG, or TGA) at the end of the coding sequence. Several of the nucleotide sequences disclosed herein are missing an initial “ATG” and/or are missing a stop codon. Any of the disclosed nucleotide sequences can be, if desired, fused to another nucleotide sequence that when operably linked to a “control element” results in the proper translation of the encoded amino acids (for example, a fusion protein). In addition, two or more nucleotide sequences can be linked by a short peptide, for example, a viral peptide.

SEQ ID NO: 1 is the nucleotide sequence of SN03 annotated in the Chlamydomonas reinhardtii wild-type strain CC-1690 21 gr mt+ genome (JGI protein ID #147817).

SEQ ID NO: 2 is the sequence of SEQ ID NO: 1 without an initial “atg” and a stop codon.

SEQ ID NO: 3 is the nucleotide sequence of SEQ ID NO: 1 codon optimized for expression in the nucleus of Chlamydomonas reinhardtii. There is no stop codon.

SEQ ID NO: 4 is the sequence of SEQ ID NO: 3 without an initial “atg”.

SEQ ID NO: 5 is the nucleotide sequence of SEQ ID NO: 3 with the addition at the 3′ end of an AgeI restriction site, a nucleotide sequence coding for a FLAG tag, a nucleotide sequence coding for a MAT tag, another AgeI restriction site, and a stop codon.

SEQ ID NO: 6 is the translated protein sequence of SEQ ID NO: 1.

SEQ ID NO: 7 is the translated protein sequence of SEQ ID NO: 5.

SEQ ID NO: 8 is the nucleotide sequence of the endogenous SN03 cDNA taken from Chlamydomonas reinhardii wild-type strain CC-1690 21 gr mt+.

SEQ ID NO: 9 is the sequence of SEQ ID NO: 8 without an initial “atg” and a stop codon.

SEQ ID NO: 10 is the sequence of SEQ ID NO: 8 with an XhoI restriction site in place of the ATG at the 5′ end, an AgeI restriction site after the final codon, a nucleotide sequence coding for a FLAG tag, a nucleotide sequence coding for a MAT tag, a six base pair sequence corresponding to the joining of XmaI and AgeI restriction sites, and a STOP codon at the 3′ end.

SEQ ID NO: 11 is the sequence of SEQ ID NO: 8 codon optimized for expression in the nucleus of Chlamydomonas reinhardtii.

SEQ ID NO: 12 is the sequence of SEQ ID NO: 11 without an initial “atg” and a stop codon.

SEQ ID NO: 13 is the sequence of SEQ ID NO: 11 with an XhoI restriction site in place of the ATG at the 5′ end, an AgeI restriction site after the final codon, a nucleotide sequence coding for a FLAG tag, a nucleotide sequence coding for a MAT tag, a six base pair sequence corresponding to the joining of XmaI and AgeI restriction sites, and a STOP codon at the 3′ end.

SEQ ID NO: 14 is the translated protein of SEQ ID NO: 8.

SEQ ID NO: 15 is the translated protein sequence of SEQ ID NO: 13.

SEQ ID NO: 16 is the nucleotide sequence of SEQ ID NO: 50 with the codons for two of the histidine residues that make up the putative zinc finger domain altered to code for threonine; specifically nucleic acid numbers 982 and 983 are changed from a CA to an AC, and nucleic acids numbers 988 and 989 are changed from a CA to an AC.

SEQ ID NO: 17 is the nucleotide sequence of SEQ ID NO: 50 with the codons for one of the histidine residues that make up the putative zinc finger domain altered to code for threonine; specifically nucleic acid numbers 1024 and 1025 are changed from a CA to an AC.

SEQ ID NO: 18 is the nucleotide sequence of SEQ ID NO: 50 with the codons for three of the histidine residues that make up the putative zinc finger domain altered to code for threonine; specifically nucleic acid numbers 982 and 983 are changed from a CA to an AC, nucleic acids numbers 988 and 989 are changed from a CA to an AC, and nucleic acid numbers 1024 and 1025 are changed from a CA to an AC.

SEQ ID NO: 19 is the translated protein of SEQ ID NO: 16.

SEQ ID NO: 20 is the translated protein of SEQ ID NO: 17.

SEQ ID NO: 21 is the translated protein of SEQ ID NO: 18.

SEQ ID NOs: 22 to 37 are primer sequences.

SEQ ID NOs: 38-41 are miRNA target nucleotide sequences.

SEQ ID NOs: 42-47 are primer sequences.

SEQ ID NO: 48 is the nucleotide sequence of BD11.

SEQ ID NO: 49 is a primer sequence.

SEQ ID NO: 50 is the sequence of SEQ ID NO: 3 with an XhoI restriction site in place of the ATG at the 5′ end, an AgeI restriction site after the final codon, a nucleotide sequence coding for a FLAG tag, a nucleotide sequence coding for a MAT tag, a six base pair sequence encoding an AgeI restriction site, and a STOP codon at the 3′ end.

SEQ ID NO: 51 is the protein sequence of SEQ ID NO: 6 without the initial “M”.

SEQ ID NO: 52 is the protein sequence of SEQ ID NO: 14 without the initial “M”.

SEQ ID NO: 53 is a nucleotide sequence comprising a mutated putative zinc finger domain.

SEQ ID NO: 54 is a nucleotide sequence comprising a mutated putative zinc finger domain.

SEQ ID NO: 55 is a nucleotide sequence comprising a mutated putative zinc finger domain.

SEQ ID NO: 56 is the translated protein sequence of SEQ ID NO: 53.

SEQ ID NO: 57 is the translated protein sequence of SEQ ID NO: 54.

SEQ ID NO: 58 is the translated protein sequence of SEQ ID NO: 55.

SEQ ID NO: 59 is a 5′ untranslated (UTR) region.

SEQ ID NO: 60 is a 3′ untranslated (UTR) region.

Lipid Trait Genes.

SEQ ID NO: 61 is the endogenous nucleotide sequence of SN02.

SEQ ID NO: 62 is the translated protein sequence of SEQ ID NO: 61.

SEQ ID NO: 63 is the codon-optimized nucleotide sequence of SN02 with additional nucleic acid sequences at both the 5′ and 3′ ends.

SEQ ID NO: 64 is SEQ ID NO: 63 without the additional nucleic acid sequences at both the 5′ and 3′ ends.

SEQ ID NO: 65 is SEQ ID NO: 61 minus the initial “ATG” and the stop codon.

SEQ ID NO: 66 is SEQ ID NO: 62 minus the initial “M”.

SEQ ID NO: 67 is the endogenous nucleotide sequence of SN03.

SEQ ID NO: 68 is the translated protein sequence of SEQ ID NO: 67.

SEQ ID NO: 69 is the codon-optimized nucleotide sequence of SN03 with additional nucleic acid sequences at both the 5′ and 3′ ends.

SEQ ID NO: 70 is SEQ ID NO: 69 without the additional nucleic acid sequences at both the 5′ and 3′ ends.

SEQ ID NO: 71 is SEQ ID NO: 67 minus the initial “ATG” and the stop codon.

SEQ ID NO: 72 is SEQ ID NO: 68 minus the initial “M”.

SEQ ID NO: 73 is the endogenous nucleotide sequence of SN08.

SEQ ID NO: 74 is the translated protein sequence of SEQ ID NO: 73.

SEQ ID NO: 75 is the codon-optimized nucleotide sequence of SN08 with additional nucleic acid sequences at both the 5′ and 3′ ends.

SEQ ID NO: 76 is SEQ ID NO: 75 without the additional nucleic acid sequences at both the 5′ and 3′ ends.

SEQ ID NO: 77 is SEQ ID NO: 73 minus the initial “ATG” and the stop codon.

SEQ ID NO: 78 is SEQ ID NO: 74 minus the initial “M”.

SEQ ID NO: 79 is the endogenous nucleotide sequence of SN09.

SEQ ID NO: 80 is the translated protein sequence of SEQ ID NO: 79.

SEQ ID NO: 81 is the codon-optimized nucleotide sequence of SN09 with additional nucleic acid sequences at both the 5′ and 3′ ends.

SEQ ID NO: 82 is SEQ ID NO: 81 without the additional nucleic acid sequences at both the 5′ and 3′ ends.

SEQ ID NO: 83 is SEQ ID NO: 79 minus the initial “ATG” and the stop codon.

SEQ ID NO: 84 is SEQ ID NO: 80 minus the initial “M”.

SEQ ID NO: 85 is the endogenous nucleotide sequence of SN11.

SEQ ID NO: 86 is the translated protein sequence of SEQ ID NO: 85.

SEQ ID NO: 87 is the codon-optimized nucleotide sequence of SN11 with additional nucleic acid sequences at both the 5′ and 3′ ends.

SEQ ID NO: 88 is SEQ ID NO: 87 without the additional nucleic acid sequences at both the 5′ and 3′ ends.

SEQ ID NO: 89 is SEQ ID NO: 85 minus the initial “ATG” and the stop codon.

SEQ ID NO: 90 is SEQ ID NO: 86 minus the initial “M”.

SEQ ID NO: 91 is the endogenous nucleotide sequence of SN21.

SEQ ID NO: 92 is the translated protein sequence of SEQ ID NO: 91.

SEQ ID NO: 93 is the codon-optimized nucleotide sequence of SN21 with additional nucleic acid sequences at both the 5′ and 3′ ends.

SEQ ID NO: 94 is SEQ ID NO: 93 without the additional nucleic acid sequences at both the 5′ and 3′ ends.

SEQ ID NO: 95 is SEQ ID NO: 91 minus the initial “ATG” and the stop codon.

SEQ ID NO: 96 is SEQ ID NO: 92 minus the initial “M”.

SEQ ID NO: 97 is the endogenous nucleotide sequence of SN26.

SEQ ID NO: 98 is the translated protein sequence of SEQ ID NO: 97.

SEQ ID NO: 99 is the codon-optimized nucleotide sequence of SN26 with additional nucleic acid sequences at both the 5′ and 3′ ends.

SEQ ID NO: 100 is SEQ ID NO: 99 without the additional nucleic acid sequences at both the 5′ and 3′ ends.

SEQ ID NO: 101 is SEQ ID NO: 97 minus the initial “ATG” and the stop codon.

SEQ ID NO: 102 is SEQ ID NO: 98 minus the initial “M”.

SEQ ID NO: 103 is the endogenous nucleotide sequence of SN39.

SEQ ID NO: 104 is the translated protein sequence of SEQ ID NO: 103.

SEQ ID NO: 105 is the codon-optimized nucleotide sequence of SN39 with additional nucleic acid sequences at both the 5′ and 3′ ends.

SEQ ID NO: 106 is SEQ ID NO: 105 without the additional nucleic acid sequences at both the 5′ and 3′ ends.

SEQ ID NO: 107 is SEQ ID NO: 103 minus the initial “ATG” and the stop codon.

SEQ ID NO: 108 is SEQ ID NO: 104 minus the initial “M”.

SEQ ID NO: 109 is the endogenous nucleotide sequence of SN71.

SEQ ID NO: 110 is the translated protein sequence of SEQ ID NO: 109.

SEQ ID NO: 111 is the codon-optimized nucleotide sequence of SN71 with additional nucleic acid sequences at both the 5′ and 3′ ends.

SEQ ID NO: 112 is SEQ ID NO: 111 without the additional nucleic acid sequences at both the 5′ and 3′ ends.

SEQ ID NO: 113 is SEQ ID NO: 109 minus the initial “ATG” and the stop codon.

SEQ ID NO: 114 is SEQ ID NO: 110 minus the initial “M”.

SEQ ID NO: 115 is the endogenous nucleotide sequence of SN75.

SEQ ID NO: 116 is the translated protein sequence of SEQ ID NO: 115.

SEQ ID NO: 117 is the codon-optimized nucleotide sequence of SN75 with additional nucleic acid sequences at both the 5′ and 3′ ends.

SEQ ID NO: 118 is SEQ ID NO: 117 without the additional nucleic acid sequences at both the 5′ and 3′ ends.

SEQ ID NO: 119 is SEQ ID NO: 115 minus the initial “ATG” and the stop codon.

SEQ ID NO: 120 is SEQ ID NO: 116 minus the initial “M”.

SEQ ID NO: 121 is the endogenous nucleotide sequence of SN80.

SEQ ID NO: 122 is the translated protein sequence of SEQ ID NO: 121.

SEQ ID NO: 123 is the codon-optimized nucleotide sequence of SN80 with additional nucleic acid sequences at both the 5′ and 3′ ends.

SEQ ID NO: 124 is SEQ ID NO: 123 without the additional nucleic acid sequences at both the 5′ and 3′ ends.

SEQ ID NO: 125 is SEQ ID NO: 121 minus the initial “ATG” and the stop codon.

SEQ ID NO: 126 is SEQ ID NO: 122 minus the initial “M”.

SEQ ID NO: 127 is the endogenous nucleotide sequence of SN81.

SEQ ID NO: 128 is the translated protein sequence of SEQ ID NO: 127.

SEQ ID NO: 129 is the codon-optimized nucleotide sequence of SN81 with additional nucleic acid sequences at both the 5′ and 3′ ends.

SEQ ID NO: 130 is SEQ ID NO: 129 without the additional nucleic acid sequences at both the 5′ and 3′ ends.

SEQ ID NO: 131 is SEQ ID NO: 127 minus the initial “ATG” and the stop codon.

SEQ ID NO: 132 is SEQ ID NO: 128 minus the initial “M”.

SEQ ID NO: 133 is the endogenous nucleotide sequence of SN84.

SEQ ID NO: 134 is the translated protein sequence of SEQ ID NO: 133.

SEQ ID NO: 135 is the codon-optimized nucleotide sequence of SN84 with additional nucleic acid sequences at both the 5′ and 3′ ends.

SEQ ID NO: 136 is SEQ ID NO: 135 without the additional nucleic acid sequences at both the 5′ and 3′ ends.

SEQ ID NO: 137 is SEQ ID NO: 133 minus the initial “ATG” and the stop codon.

SEQ ID NO: 138 is SEQ ID NO: 134 minus the initial “M”.

SEQ ID NO: 139 is the endogenous nucleotide sequence of SN87.

SEQ ID NO: 140 is the translated protein sequence of SEQ ID NO: 139.

SEQ ID NO: 141 is the codon-optimized nucleotide sequence of SN87 with additional nucleic acid sequences at both the 5′ and 3′ ends.

SEQ ID NO: 142 is SEQ ID NO: 141 without the additional nucleic acid sequences at both the 5′ and 3′ ends.

SEQ ID NO: 143 is SEQ ID NO: 139 minus the initial “ATG” and the stop codon.

SEQ ID NO: 144 is SEQ ID NO: 140 minus the initial “M”.

SEQ ID NO: 145 is the endogenous nucleotide sequence of SN91.

SEQ ID NO: 146 is the translated protein sequence of SEQ ID NO: 145.

SEQ ID NO: 147 is the codon-optimized nucleotide sequence of SN91 with additional nucleic acid sequences at both the 5′ and 3′ ends.

SEQ ID NO: 148 is SEQ ID NO: 147 without the additional nucleic acid sequences at both the 5′ and 3′ ends.

SEQ ID NO: 149 is SEQ ID NO: 145 minus the initial “ATG” and the stop codon.

SEQ ID NO: 150 is SEQ ID NO: 146 minus the initial “M”.

SEQ ID NO: 151 is the endogenous nucleotide sequence of SN108.

SEQ ID NO: 152 is the translated protein sequence of SEQ ID NO: 151.

SEQ ID NO: 153 is the codon-optimized nucleotide sequence of SN108 with additional nucleic acid sequences at both the 5′ and 3′ ends.

SEQ ID NO: 154 is SEQ ID NO: 153 without the additional nucleic acid sequences at both the 5′ and 3′ ends.

SEQ ID NO: 155 is SEQ ID NO: 151 minus the initial “ATG” and the stop codon.

SEQ ID NO: 156 is SEQ ID NO: 152 minus the initial “M”.

SEQ ID NO: 157 is the endogenous nucleotide sequence of SN110.

SEQ ID NO: 158 is the translated protein sequence of SEQ ID NO: 157.

SEQ ID NO: 159 is the codon-optimized nucleotide sequence of SN110 with additional nucleic acid sequences at both the 5′ and 3′ ends.

SEQ ID NO: 160 is SEQ ID NO: 159 without the additional nucleic acid sequences at both the 5′ and 3′ ends.

SEQ ID NO: 161 is SEQ ID NO: 157 minus the initial “ATG” and the stop codon.

SEQ ID NO: 162 is SEQ ID NO: 158 minus the initial “M”.

SEQ ID NO: 163 is the endogenous nucleotide sequence of SN120.

SEQ ID NO: 164 is the translated protein sequence of SEQ ID NO: 163.

SEQ ID NO: 165 is the codon-optimized nucleotide sequence of SN120 with additional nucleic acid sequences at both the 5′ and 3′ ends.

SEQ ID NO: 166 is SEQ ID NO: 165 without the additional nucleic acid sequences at both the 5′ and 3′ ends.

SEQ ID NO: 167 is SEQ ID NO: 163 minus the initial “ATG” and the stop codon.

SEQ ID NO: 168 is SEQ ID NO: 164 minus the initial “M”.

SEQ ID NO: 169 is the endogenous nucleotide sequence of SN124.

SEQ ID NO: 170 is the translated protein sequence of SEQ ID NO: 169.

SEQ ID NO: 171 is the codon-optimized nucleotide sequence of SN124 with additional nucleic acid sequences at both the 5′ and 3′ ends.

SEQ ID NO: 172 is SEQ ID NO: 171 without the additional nucleic acid sequences at both the 5′ and 3′ ends.

SEQ ID NO: 173 is SEQ ID NO: 169 minus the initial “ATG” and the stop codon.

SEQ ID NO: 174 is SEQ ID NO: 170 minus the initial “M”.

Growth Trait Genes.

SEQ ID NO: 175 is the endogenous nucleotide sequence of SN01.

SEQ ID NO: 176 is the translated protein sequence of SEQ ID NO: 175.

SEQ ID NO: 177 is the codon-optimized nucleotide sequence of SN01 with additional nucleic acid sequences at both the 5′ and 3′ ends.

SEQ ID NO: 178 is SEQ ID NO: 177 without the additional nucleic acid sequences at both the 5′ and 3′ ends.

SEQ ID NO: 179 is SEQ ID NO: 175 minus the initial “ATG” and the stop codon.

SEQ ID NO: 180 is SEQ ID NO: 176 minus the initial “M”.

SEQ ID NO: 181 is the endogenous nucleotide sequence of SN06.

SEQ ID NO: 182 is the translated protein sequence of SEQ ID NO: 181.

SEQ ID NO: 183 is the codon-optimized nucleotide sequence of SN06 with additional nucleic acid sequences at both the 5′ and 3′ ends.

SEQ ID NO: 184 is SEQ ID NO: 183 without the additional nucleic acid sequences at both the 5′ and 3′ ends.

SEQ ID NO: 185 is SEQ ID NO: 181 minus the initial “ATG” and the stop codon.

SEQ ID NO: 186 is SEQ ID NO: 182 minus the initial “M”.

SEQ ID NO: 187 is the endogenous nucleotide sequence of SN24.

SEQ ID NO: 188 is the translated protein sequence of SEQ ID NO: 187.

SEQ ID NO: 189 is the codon-optimized nucleotide sequence of SN24 with additional nucleic acid sequences at both the 5′ and 3′ ends.

SEQ ID NO: 190 is SEQ ID NO: 189 without the additional nucleic acid sequences at both the 5′ and 3′ ends.

SEQ ID NO: 191 is SEQ ID NO: 187 minus the initial “ATG” and the stop codon.

SEQ ID NO: 192 is SEQ ID NO: 188 minus the initial “M”.

SEQ ID NO: 193 is the endogenous nucleotide sequence of SN25.

SEQ ID NO: 194 is the translated protein sequence of SEQ ID NO: 193.

SEQ ID NO: 195 is the codon-optimized nucleotide sequence of SN25 with additional nucleic acid sequences at both the 5′ and 3′ ends.

SEQ ID NO: 196 is SEQ ID NO: 195 without the additional nucleic acid sequences at both the 5′ and 3′ ends.

SEQ ID NO: 197 is SEQ ID NO: 193 minus the initial “ATG” and the stop codon.

SEQ ID NO: 198 is SEQ ID NO: 194 minus the initial “M”.

SEQ ID NO: 199 is the endogenous nucleotide sequence of SN28.

SEQ ID NO: 200 is the translated protein sequence of SEQ ID NO: 199.

SEQ ID NO: 201 is the codon-optimized nucleotide sequence of SN28 with additional nucleic acid sequences at both the 5′ and 3′ ends.

SEQ ID NO: 202 is SEQ ID NO: 201 without the additional nucleic acid sequences at both the 5′ and 3′ ends.

SEQ ID NO: 203 is SEQ ID NO: 199 minus the initial “ATG” and the stop codon.

SEQ ID NO: 204 is SEQ ID NO: 200 minus the initial “M”.

SEQ ID NO: 205 is the endogenous nucleotide sequence of SN42.

SEQ ID NO: 206 is the translated protein sequence of SEQ ID NO: 205.

SEQ ID NO: 207 is the codon-optimized nucleotide sequence of SN42 with additional nucleic acid sequences at both the 5′ and 3′ ends.

SEQ ID NO: 208 is SEQ ID NO: 207 without the additional nucleic acid sequences at both the 5′ and 3′ ends.

SEQ ID NO: 209 is SEQ ID NO: 205 minus the initial “ATG” and the stop codon.

SEQ ID NO: 210 is SEQ ID NO: 206 minus the initial “M”.

SEQ ID NO: 211 is the endogenous nucleotide sequence of SN46.

SEQ ID NO: 212 is the translated protein sequence of SEQ ID NO: 211.

SEQ ID NO: 213 is the codon-optimized nucleotide sequence of SN46 with additional nucleic acid sequences at both the 5′ and 3′ ends.

SEQ ID NO: 214 is SEQ ID NO: 213 without the additional nucleic acid sequences at both the 5′ and 3′ ends.

SEQ ID NO: 215 is SEQ ID NO: 211 minus the initial “ATG” and the stop codon.

SEQ ID NO: 216 is SEQ ID NO: 212 minus the initial “M”.

SEQ ID NO: 217 is the endogenous nucleotide sequence of SN47.

SEQ ID NO: 218 is the translated protein sequence of SEQ ID NO: 217.

SEQ ID NO: 219 is the codon-optimized nucleotide sequence of SN47 with additional nucleic acid sequences at both the 5′ and 3′ ends.

SEQ ID NO: 220 is SEQ ID NO: 219 without the additional nucleic acid sequences at both the 5′ and 3′ ends.

SEQ ID NO: 221 is SEQ ID NO: 217 minus the initial “ATG” and the stop codon.

SEQ ID NO: 222 is SEQ ID NO: 218 minus the initial “M”.

SEQ ID NO: 223 is the endogenous nucleotide sequence of SN55.

SEQ ID NO: 224 is the translated protein sequence of SEQ ID NO: 223.

SEQ ID NO: 225 is the codon-optimized nucleotide sequence of SN55 with additional nucleic acid sequences at both the 5′ and 3′ ends.

SEQ ID NO: 226 is SEQ ID NO: 225 without the additional nucleic acid sequences at both the 5′ and 3′ ends.

SEQ ID NO: 227 is SEQ ID NO: 223 minus the initial “ATG” and the stop codon.

SEQ ID NO: 228 is SEQ ID NO: 224 minus the initial “M”.

SEQ ID NO: 229 is the endogenous nucleotide sequence of SN57.

SEQ ID NO: 230 is the translated protein sequence of SEQ ID NO: 229.

SEQ ID NO: 231 is the codon-optimized nucleotide sequence of SN57 with additional nucleic acid sequences at both the 5′ and 3′ ends.

SEQ ID NO: 232 is SEQ ID NO: 231 without the additional nucleic acid sequences at both the 5′ and 3′ ends.

SEQ ID NO: 233 is SEQ ID NO: 229 minus the initial “ATG” and the stop codon.

SEQ ID NO: 234 is SEQ ID NO: 230 minus the initial “M”.

SEQ ID NO: 235 is the endogenous nucleotide sequence of SNS9.

SEQ ID NO: 236 is the translated protein sequence of SEQ ID NO: 235.

SEQ ID NO: 237 is the codon-optimized nucleotide sequence of SNS9 with additional nucleic acid sequences at both the 5′ and 3′ ends.

SEQ ID NO: 238 is SEQ ID NO: 237 without the additional nucleic acid sequences at both the 5′ and 3′ ends.

SEQ ID NO: 239 is SEQ ID NO: 235 minus the initial “ATG” and the stop codon.

SEQ ID NO: 240 is SEQ ID NO: 236 minus the initial “M”.

SEQ ID NO: 241 is the endogenous nucleotide sequence of SN64.

SEQ ID NO: 242 is the translated protein sequence of SEQ ID NO: 241.

SEQ ID NO: 243 is the codon-optimized nucleotide sequence of SN64 with additional nucleic acid sequences at both the 5′ and 3′ ends.

SEQ ID NO: 244 is SEQ ID NO: 243 without the additional nucleic acid sequences at both the 5′ and 3′ ends.

SEQ ID NO: 245 is SEQ ID NO: 241 minus the initial “ATG” and the stop codon.

SEQ ID NO: 246 is SEQ ID NO: 242 minus the initial “M”.

SEQ ID NO: 247 is the endogenous nucleotide sequence of SN69.

SEQ ID NO: 248 is the translated protein sequence of SEQ ID NO: 247.

SEQ ID NO: 249 is the codon-optimized nucleotide sequence of SN69 with additional nucleic acid sequences at both the 5′ and 3′ ends.

SEQ ID NO: 250 is SEQ ID NO: 249 without the additional nucleic acid sequences at both the 5′ and 3′ ends.

SEQ ID NO: 251 is SEQ ID NO: 247 minus the initial “ATG” and the stop codon.

SEQ ID NO: 252 is SEQ ID NO: 248 minus the initial “M”.

SEQ ID NO: 253 is the endogenous nucleotide sequence of SN76.

SEQ ID NO: 254 is the translated protein sequence of SEQ ID NO: 253.

SEQ ID NO: 255 is the codon-optimized nucleotide sequence of SN76 with additional nucleic acid sequences at both the 5′ and 3′ ends.

SEQ ID NO: 256 is SEQ ID NO: 255 without the additional nucleic acid sequences at both the 5′ and 3′ ends.

SEQ ID NO: 257 is SEQ ID NO: 253 minus the initial “ATG” and the stop codon.

SEQ ID NO: 258 is SEQ ID NO: 254 minus the initial “M”.

SEQ ID NO: 259 is the endogenous nucleotide sequence of SN78.

SEQ ID NO: 260 is the translated protein sequence of SEQ ID NO: 259.

SEQ ID NO: 261 is the codon-optimized nucleotide sequence of SN78 with additional nucleic acid sequences at both the 5′ and 3′ ends.

SEQ ID NO: 262 is SEQ ID NO: 261 without the additional nucleic acid sequences at both the 5′ and 3′ ends.

SEQ ID NO: 263 is SEQ ID NO: 259 minus the initial “ATG” and the stop codon.

SEQ ID NO: 264 is SEQ ID NO: 260 minus the initial “M”.

SEQ ID NO: 265 is the endogenous nucleotide sequence of SN79.

SEQ ID NO: 266 is the translated protein sequence of SEQ ID NO: 265.

SEQ ID NO: 267 is the codon-optimized nucleotide sequence of SN79 with additional nucleic acid sequences at both the 5′ and 3′ ends.

SEQ ID NO: 268 is SEQ ID NO: 267 without the additional nucleic acid sequences at both the 5′ and 3′ ends.

SEQ ID NO: 269 is SEQ ID NO: 265 minus the initial “ATG” and the stop codon.

SEQ ID NO: 270 is SEQ ID NO: 266 minus the initial “M”.

SEQ ID NO: 271 is the endogenous nucleotide sequence of SN82.

SEQ ID NO: 272 is the translated protein sequence of SEQ ID NO: 271.

SEQ ID NO: 273 is the codon-optimized nucleotide sequence of SN82 with additional nucleic acid sequences at both the 5′ and 3′ ends.

SEQ ID NO: 274 is SEQ ID NO: 273 without the additional nucleic acid sequences at both the 5′ and 3′ ends.

SEQ ID NO: 275 is SEQ ID NO: 271 minus the initial “ATG” and the stop codon.

SEQ ID NO: 276 is SEQ ID NO: 272 minus the initial “M”.

SEQ ID NO: 277 is the endogenous nucleotide sequence of SN111.

SEQ ID NO: 278 is the translated protein sequence of SEQ ID NO: 277.

SEQ ID NO: 279 is the codon-optimized nucleotide sequence of SN111 with additional nucleic acid sequences at both the 5′ and 3′ ends.

SEQ ID NO: 280 is SEQ ID NO: 279 without the additional nucleic acid sequences at both the 5′ and 3′ ends.

SEQ ID NO: 281 is SEQ ID NO: 277 minus the initial “ATG” and the stop codon.

SEQ ID NO: 282 is SEQ ID NO: 278 minus the initial “M”.

SEQ ID NO: 283 is the endogenous nucleotide sequence of SN118.

SEQ ID NO: 284 is the translated protein sequence of SEQ ID NO: 283.

SEQ ID NO: 285 is the codon-optimized nucleotide sequence of SN118 with additional nucleic acid sequences at both the 5′ and 3′ ends.

SEQ ID NO: 286 is SEQ ID NO: 285 without the additional nucleic acid sequences at both the 5′ and 3′ ends.

SEQ ID NO: 287 is SEQ ID NO: 283 minus the initial “ATG” and the stop codon.

SEQ ID NO: 288 is SEQ ID NO: 284 minus the initial “M”.

SEQ ID NO: 289 is the endogenous nucleotide sequence of SN122.

SEQ ID NO: 290 is the translated protein sequence of SEQ ID NO: 289.

SEQ ID NO: 291 is the codon-optimized nucleotide sequence of SN122 with additional nucleic acid sequences at both the 5′ and 3′ ends.

SEQ ID NO: 292 is SEQ ID NO: 291 without the additional nucleic acid sequences at both the 5′ and 3′ ends.

SEQ ID NO: 293 is SEQ ID NO: 289 minus the initial “ATG” and the stop codon.

SEQ ID NO: 294 is SEQ ID NO: 290 minus the initial “M”.

SEQ ID NO: 295 is the endogenous nucleotide sequence of SN128.

SEQ ID NO: 296 is the translated protein sequence of SEQ ID NO: 295.

SEQ ID NO: 297 is the codon-optimized nucleotide sequence of SN128 with additional nucleic acid sequences at both the 5′ and 3′ ends.

SEQ ID NO: 298 is SEQ ID NO: 297 without the additional nucleic acid sequences at both the 5′ and 3′ ends.

SEQ ID NO: 299 is SEQ ID NO: 295 minus the initial “ATG” and the stop codon.

SEQ ID NO: 300 is SEQ ID NO: 296 minus the initial “M”.

Media's Used and Levels of Ammonium

Tris-acetate-phosphate (TAP) media contains a final concentration of 7.5 mM NH₄Cl. High-salt-media (HSM) contains a final concentration of 7.5 mM NH₄Cl (for example, as described in Harris (2009) The Chlamydomonas Sourcebook, Academic Press, San Diego, Calif.) Modified artificial seawater media (MASM) contains a final concentration of 11.8 mM NaNO₃ and 0.5 mM NH₄Cl. The final NH₄Cl concentration in TAP or HSM media can be varied, for example, so that the final NH₄Cl concentration is about 0.5 mM to about 7.5 mM.

The interrelation between the different nitrogen limitation phenotypes in algae (i.e., increased lipid, breakdown of photosystem, decreased growth, and mating induction) has long been assumed to be directly linked. Efforts to separate, for example, the lipid increase from reduced growth have met with failure, leading to the accepted hypothesis that nutrient flux is fixed and increasing usage for one pathway (e.g., lipid) always leads to a concomitant reduction in another pathway (e.g., growth). Under environmental stress, many algae modify their biosynthetic pathways to accumulate higher levels of lipid, with concurrent changes in the profile of accumulated lipids as well.

We have identified an mRNA encoding a protein (SN03) in Chlamydomonas reinhardtii wild-type strain CC-1690 21 gr mt+ whose expression is up regulated upon nitrogen starvation (stress conditions). SN03 acts as a lipid trigger; over expression of this protein in algae leads to increases in lipid levels with little impact on other nitrogen limitation phenotypes. Over-expression of this protein in algae results in an increase in total extractable fats and a change in the lipid profile that is similar to the change in profile induced by nitrogen starvation. Thus, we have triggered stress-induced lipid accumulation in the absence of external stress.

Algae were analyzed for total gravimetric lipids by methanol/methyl-tert-butyl ether (MTBE) extraction according to a modified Bligh Dyer method (as described in Matyash V., et al. (2008) Journal of Lipid Research 49:1137-1146) or by the original Bligh Dyer method (as described in BLIGH and DYER. (1959) Can J Biochem Physiol vol. 37 (8) pp. 911-7). These total extractable fats are analyzed by HPLC or NMR to determine the distribution of lipids among various lipid classes (lipid profile).

Overexpression of SN03 in a host will allow for an increased level of extractable lipids to make, for example, biofuels. The identification of SN03 will allow one skilled in the art to determine the various pathways affected by changes in nitrogen levels that are responsible for the various downstream phenotypes. In addition, the methods described herein will allow for the identification of proteins that are homologous to SN03.

In addition, we have identified a number of mRNAs encoding proteins in Chlamydomonas reinhardtii wild-type strain CC-1690 21 gr mt+ whose expression is up or down regulated upon nitrogen starvation (stress conditions). Some of these mRNAs are also up or down regulated in a Chlamydomonas strain overexpressing the SN03 protein. Individual overexpression of these proteins in algae result in phenotypes related to those induced by nitrogen stress in algae. These phenotypes include an increase in total extractable fats, a change in the lipid content or profile and/or a change in the growth or productivity of the transformed organism. Thus, we have triggered stress related phenotypes in the absence of external stress.

Algae

Oxygenic photosynthetic microalgae and cyanobacteria (for simplicity, algae) represent an extremely diverse, yet highly specialized group of micro-organisms that live in diverse ecological habitats such as freshwater, brackish, marine, and hyper-saline, with a range of temperatures and pH, and unique nutrient availabilities (for example, as described in Falkowski, P. G., and Raven. J. A. Aquatic Photosynthesis, Malden, Mass.: Blackwell Science). With over 40,000 species already identified and with many more yet to be identified, algae are classified in multiple major groupings as follows: cyanobacteria (Cyanophyceae), green algae (Chlorophyceae), diatoms (Bacillariophyceae), yellow-green algae (Xanthophyceae), golden algae (Chrysophyceae), red algae (Rhodophyceae), brown algae (Phaeophyceae), dinoflagellates (Dinophyceae), and ‘pico-plankton’ (Prasinophyceae and Eustigmatophyceae). Several additional divisions and classes of unicellular algae have been described, and details of their structure and biology are available (for example, as described in Van den Hoek et al., 1995). Thousands of species and strains of these algal taxa are currently maintained in culture collections throughout the world (http://www.utex.org; http://ccmp.bigelow.org; http://www.ccap.ac.uk; http://www.marine.csiro.au/microalgae; http://wdcm.nig.ac.jp/hpcc.html). In addition, there are many species of macroalgae, for example, Cladophora glomerata and Fucus vesiculosus.

The ability of algae to survive or proliferate over a wide range of environmental conditions is, to a large extent, reflected in the tremendous diversity and sometimes unusual pattern of cellular lipids that algae can produce as well as the ability to modify lipid metabolism efficiently in response to changes in environmental conditions (for example, as described in Guschina, I. A. and Harwood, J. L. (2006) Prog. Lipid Res. 45, 160-186; Thompson, G. A. (1996) Biochim. Biophys. Acta, 1302, 17-45; and Wada, H. and Murata, N. (1998) Membrane lipids in cyanobacteria. In Lipids in Photosynthesis: Structure, Function and Genetics (Siegenthaler, P. A. and Murata, N., eds). Dordrecht, The Netherlands: Kluwer Academic Publishers, pp. 65-81). The lipids that algae produce may include, but are not limited to, neutral lipids, polar lipids, wax esters, sterols and hydrocarbons, as well as prenyl derivatives such as tocopherols, carotenoids, terpenes, quinines, and phytylated pyrrole derivatives such as the chlorophylls.

Under optimal conditions of growth, algae synthesize fatty acids principally for esterification into glycerol-based membrane lipids, which constitute about 5-20% of their dry cell weight (DCW). Fatty acids include medium-chain (C10-C14), long-chain (C16-18), and very-long-chain (C20 or more) species and fatty acid derivatives. The major membrane lipids are the glycosylglycerides (e.g. monogalactosyldiacylglycerol, digalactosyldiacylglycerol and sulfoquinovosyldiacylglycerol), which are enriched in the chloroplast, together with significant amounts of phosphoglycerides (e.g. phosphatidylethanolamine, PE, and phosphatidylglycerol, PG), which mainly reside in the plasma membrane and many endoplasmic membrane systems (for example, as described in Guckert, J. B. and Cooksey. K. E. (1990) J. Phycol. 26, 72-79; Harwood, J. L. (1998) Membrane lipids in algae. In Lipids in Photosynthesis: Structure, Function and Genetics (Siegenthaler, P. A. and Murata, N., eds). Dordrecht. The Netherlands: Kluwer Academic Publishers, pp. 53-64: Pohl, P. and Zurheide, F. (1979) Fatty acids and lipids of marine algae and the control of their biosynthesis by environmental factors. In Marine Algae in Pharmaceutical Science (Hoppe, H. A. Levring, T. and Tanaka, Y., eds). Berlin: Walter de Gruyter, pp. 473-523; Pohl, P. and Zurheide, F. (1979) Control of fatty acid and lipid formation in Baltic marine algae by environmental factors. In Advances in the Biochemistry and Physiology of Plant Lipids (Appelqvist, L. A. and Liljenberg, C., eds). Amsterdam: Elsevier, pp. 427-432; and Wada, H. and Murata, N. (1998) Membrane lipids in cyanobacteria. In Lipids in Photosynthesis: Structure, Function and Genetics (Siegenthaler, P. A. and Murata, N., eds). Dordrecht, The Netherlands: Kluwer Academic Publishers, pp. 65-81). The major constituents of the membrane glycerolipids are various kinds of fatty acids that are polyunsaturated and derived through aerobic desaturation and chain elongation from the ‘precursor’ fatty acids palmitic (16:0) and oleic (18:1ω9) acids (for example, as described in Erwin, J. A. (1973) Comparative biochemistry of fatty acids in eukaryotic microorganisms. In Lipids and Biomembranes of Eukaryotic Microorganisms (Erwin, J. A., ed.) New York: Academic Press, pp. 141-143).

Under unfavorable environmental or stress conditions for growth, however, many algae alter their lipid biosynthetic pathways towards the formation and accumulation of neutral lipids (20-50% DCW), mainly in the form of triacylglycerol (TAG). Unlike the glycerolipids found in membranes, TAGs do not perform a structural role but instead serve primarily as a storage form of carbon and energy. However, there is some evidence suggesting that, in algae, the TAG biosynthesis pathway may play a more active role in the stress response, in addition to functioning as a carbon and energy storage under environmental stress conditions. Unlike higher plants where individual classes of lipid may be synthesized and localized in a specific cell, tissue or organ, many of these different types of lipids occur in a single algal cell. After being synthesized, TAGs are deposited in densely packed lipid bodies located in the cytoplasm of the algal cell, although formation and accumulation of lipid bodies also occurs in the inter-thylakoid space of the chloroplast in certain green algae, such as Dunaliella bardawil (for example, as described in Ben-Amotz, A., et al. (1989) Plant Physiol. 91, 1040-1043). In the latter case, the chloroplastic lipid bodies are referred to as plastoglobuli. Hydrocarbons are another type of neutral lipid that can be found in algae at quantities generally <5% DCW (for example, as described in Lee, R. F. and Loeblich, A. R. III (1971) Phytochemistry, 10, 593-602). The colonial green alga, Botryococcus braunii, has been shown to produce, under adverse environmental conditions, large quantities (up to 80% DCW) of very-long-chain (C23-C40) hydrocarbons, similar to those found in petroleum.

Lipid and Triacylglycerol Content

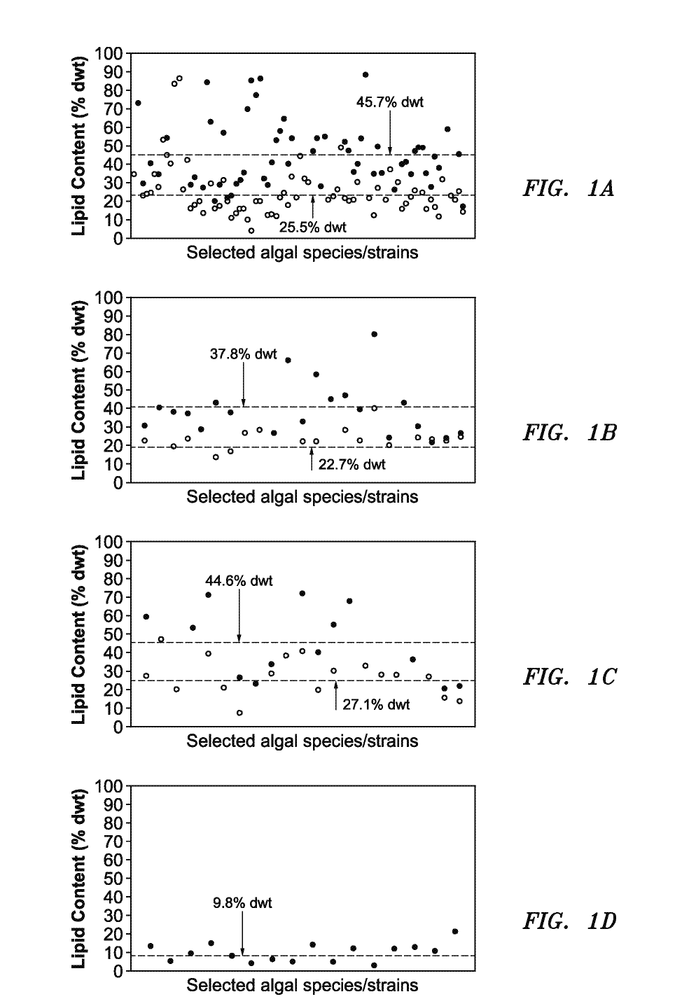

The majority of photosynthetic micro-organisms routinely used in the laboratory (e.g. Chlamydomonas reinhardtii) were selected because of ease of cultivation, or as genetic model systems for studying photosynthesis (for example, as described in Grossman et al., 2007, Curr. Opin. Plant Biol. 10, 190-198; and Merchant et al., 2007, Science, 318, 245-251). These few organisms were not selected for optimal lipid production. Therefore, examination of lipid synthesis and accumulation in diverse organisms has the potential for insights into new mechanisms to enhance lipid production. Over the past few decades, several thousand algae, and cyanobacterial species, have been screened for high lipid content, of which several hundred oleaginous species have been isolated and characterized under laboratory and/or outdoor culture conditions. Oleaginous algae can be found among diverse taxonomic groups, and the total lipid content may vary noticeably among individual species or strains within and between taxonomic groups. Of the strains examined, green algae represent the largest taxonomic group from which oleaginous candidates have been identified. This may not be because green algae naturally contain considerably more lipids than other algal taxa, but rather because many green algae are ubiquitous in diverse natural habitats, can easily be isolated, and generally grow faster than species from other taxonomic groups under laboratory conditions. FIG. 1(a) summarizes the total lipid contents of oleaginous green algae reported in the literature. Each data point represents the total lipid of an individual species or strain grown under optimal culture conditions. Oleaginous green algae show an average total lipid content of 25.5% DCW. The lipid content increases considerably (doubles or triples) when the cells are subjected to unfavorable culture conditions, such as photo-oxidative stress or nutrient starvation. On average, an increase in total lipids to 45.7% DCW was obtained from an oleaginous green algae grown under stress conditions. An effort was made to determine whether green algae at the genus level exhibit different capacities to synthesize and accumulate lipids. Statistical analysis of various oleaginous green algae indicated no significant differences. The intrinsic ability to produce large quantities of lipid and oil is species/strain-specific, rather than genus-specific (for example, as described in Hu et al., 2006, Biodiesel from Algae: Lessons Learned Over the Past 60 Years and Future Perspectives. Juneau, Ak.: Annual Meeting of the Phycological Society of America, July 7-12, pp. 40-41 (Abstract)).

FIG. 1(b) illustrates the lipid content of oleaginous diatoms of freshwater and marine origin grown under normal and stress culture conditions (for example, as described in Hu et al., 2006, Biodiesel from Algae: Lessons Learned Over the Past 60 Years and Future Perspectives. Juneau. Ak.: Annual Meeting of the Phycological Society of America, July 7-12, pp. 40-41 (Abstract)). Statistical analysis indicated that the average lipid content of an oleaginous diatom was 22.7% DCW when maintained under normal growth conditions, whereas a total lipid content of 44.6% DCW was achievable under stress conditions.

FIG. 1(c) shows the lipid content of oleaginous algae identified as chrysophytes, haptophytes, eustigmatophytes, dinophytes, xanthophytes, or rhodophytes (for example, as described in Hu et al., 2006, Biodiesel from Algae: Lessons Learned Over the Past 60 Years and Future Perspectives. Juneau, Ak.: Annual Meeting of the Phycological Society of America, July 7-12, pp. 40-41 (Abstract)). Similar to oleaginous green algae and diatoms, these species/strains show average total lipid contents of 27.1% and 44.6% DCW under normal and stress culture conditions, respectively.

The increase in total lipids in aging algal cells or cells maintained under various stress conditions consisted primarily of neutral lipids, mainly TAGs. This was due to the shift in lipid metabolism from membrane lipid synthesis to the storage of neutral lipids. De novo biosynthesis and conversion of certain existing membrane polar lipids into triacylglycerols may contribute to the overall increase in TAG. As a result, TAGs may account for as much as 80% of the total lipid content in the cell (for example, as described in Kathen, 1949, Arch. Mikrobiol. 14, 602-634; Klyachko-Gurvich, 1974, Soviet Plant Physiol. 21, 611-618; Suen et al., 1987, J. Phycol. 23, 289-297; Tonon et al., 2002, Phytochemistry 61, 15-24; and Tornabene et al., 1983, Enzyme Microbiol. Technol. 5, 435-440).

Cyanobacteria have also been subjected to screening for lipid production (for example, as described in Basova. 2005. Int. J. Algae. 7, 33-57; and Cobelas and Lechado, 1989, Grasas y Aceites, 40, 118-145). Unfortunately, considerable amounts of total lipids have not been found in cyanophycean organisms examined in the laboratory (FIG. 1d), and the accumulation of neutral lipid triacylglycerols has not been observed in naturally occurring cyanobacteria.

Fatty Acid Composition

Algae synthesize fatty acids as building blocks for the formation of various types of lipids. The most commonly synthesized fatty acids have chain lengths that range from C16 to C18, similar to those of higher plants (for example, as described in Ohlrogge and Browse, 1995, Plant Cell. 7, 957-970). Fatty acids are either saturated or unsaturated, and unsaturated fatty acids may vary in the number and position of double bonds on the carbon chain backbone. In general, saturated and mono-unsaturated fatty acids are predominant in most algae examined (for example, as described in Borowitzka. 1988. Fats, oils and hydrocarbons. In Microalgal Biotechnology (Borowitzka, M. A. and Borowitzka, L. J., eds). Cambridge, UK: Cambridge University Press, pp. 257-287). Specifically, the major fatty acids are C16:0 and C16:1 in the Bacillariophyceae, C16:0 and C18:1 in the Chlorophyceae (Chlamydomonas sp., Dunelialla sp., and Scenedesmus sp.), C16:0 and C18:1 in the Euglenophyceae, C16:0, C16:1 and C18:1 in the Chrysophyceae, C16:0 and C20:1 in the Cryptophyceae, C16:0 and C18:1 in the Eustigmatophyceae, C16:0 and C18:1 in the Prasinophyceae, C16:0 in the Dinophyceae, C16:0, C16:1 and C18:1 in the Prymnesiophyceae, C16:0 in the Rhodophyceae, C14:0, C16:0 and C16:1 in the Xanthophyceae, and C16:0, C16:1 and C18:1 in cyanobacteria (for example, as described in Cobelas and Lechado, 1989, Grasas y Aceites, 40, 118-145.

Polyunsaturated fatty acids (PUFAs) contain two or more double bonds. Based on the number of double bonds, individual fatty acids are named dienoic, trienoic, tetraenoic, pentaenoic, and hexaenoic fatty acids. Also, depending on the position of the first double bond from the terminal methyl end (x) of the carbon chain, a fatty acid may be either an ×3 PUFA (i.e. the third carbon from the end of the fatty acid) or an ×6 PUFAs (i.e. the sixth carbon from the end of the fatty acid). The major PUFAs are C20:5×3 and C22:6×3 in Bacillarilophyceae, C18:2 and C18:3×3 in green algae, C18:2 and C18:3×3 in Euglenophyceae, C20:5, C22:5 and C22:6 in Chrysophyceae, C18:3×3, 18:4 and C20:5 in Cryptophyceae, C20:3 and C20:4×3 in Eustigmatophyceae, C18: 3×3 and C20:5 in Prasinophyceae, C18:5×3 and C22:6×3 in Dinophyceae, C18:2, C18:3×3 and C22:6×3 in Prymnesiophyceae, C18:2 and C20:5 in Rhodophyceae, C16:3 and C20:5 in Xanthophyceae, and C16:0, C18:2 and C18:3×3 in cyanobacteria (for example, as described in Basova, 2005, Int. J. Algae, 7, 33-57; and Cobelas and Lechado, 1989, Grasas y Aceites, 40, 118-145).

In contrast to higher plants, greater variation in fatty acid composition is found in algal taxa. Some algae and cyanobacteria possess the ability to synthesize medium-chain fatty acids (e.g. C10, C12 and C14) as predominant species, whereas others produce very-long-chain fatty acids (>C20). For instance, a C10 fatty acid comprising 27-50% of the total fatty acids was found in the filamentous cyanobacterium Trichodesmium erythraeum (for example, as described in Parker et al., 1967, Science, 155, 707-708), and a C14 fatty acid makes up nearly 70% of the total fatty acids in the golden alga Prymnesium parvum (for example, as described in Lee and Loeblich, 1971, Phytochemistry, 10, 593-602). Another distinguishing feature of some algae is the large amounts of very-long-chain PUFAs. For example, in the green alga Parietochloris incise (as described in Bigogno et al., 2002, Phytochemistry, 60, 497-503), the diatom Phaeodactylum tricornutum and the dinoflagellate Crypthecodinium cohnii (as described in De Swaaf et al., 1999, J. Biotechnol. 70, 185-192), the very-long-chain fatty acids arachidonic acid (C20:4×6), eicosapentaenoic acid (C20:5×3), or docosahexaenoic acid (C22:6×3), are the major fatty acid species accounting for 33.6-42.5%, approximately 30%, and 30-50%, of the total fatty acid content of the three species, respectively.

It should be noted that much of the data provided previously comes from the limited number of species of algae that have been examined to date, and most of the analyses of fatty acid composition from algae have used total lipid extracts rather than examining individual lipid classes. Therefore, these data represent generalities, and deviations should be expected. This may explain why some fatty acids seem to occur almost exclusively in an individual algal taxon. In addition, the fatty acid composition of algae can vary both quantitatively and qualitatively with their physiological status and culture conditions.

Biosynthesis of Fatty Acids and Triacylglycerols

Lipid metabolism, particularly the biosynthetic pathways of fatty acids and TAG, has been poorly studied in algae in comparison to higher plants. Based upon the sequence homology and some shared biochemical characteristics of a number of genes and/or enzymes isolated from algae and higher plants that are involved in lipid metabolism, it is generally believed that the basic pathways of fatty acid and TAG biosynthesis in algae are directly analogous to those demonstrated in higher plants.

Fatty Acid Biosynthesis

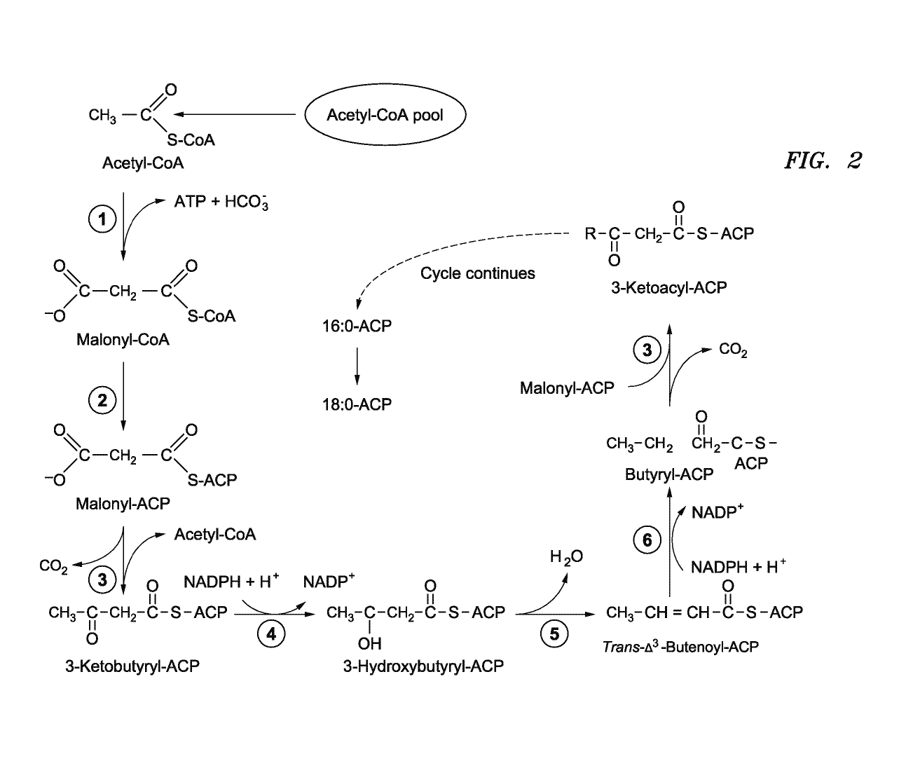

In algae, the de novo synthesis of fatty acids occurs primarily in the chloroplast. A generalized scheme for fatty acid biosynthesis is shown in FIG. 2. The pathway produces a 16- or 18-carbon fatty acid or both. These are then used as the precursors for the synthesis of chloroplast and other cellular membranes as well as for the synthesis of neutral storage lipids, mainly TAGs, which can accumulate under adverse environmental or sub-optimal growth conditions.

The committed step in fatty acid synthesis is the conversion of acetyl CoA to malonyl CoA, catalyzed by acetyl CoA carboxylase (ACCase). In the chloroplast, photosynthesis provides an endogenous source of acetyl CoA, and more than one pathway may contribute to maintaining the acetyl CoA pool. In oil seed plants, a major route of carbon flux to fatty acid synthesis may involve cytosolic glycolysis to phosphoenolpyruvate (PEP), which is then preferentially transported from the cytosol to the plastid, where it is converted to pyruvate and consequently to acetyl CoA (for example, as described in Baud et al., 2007, Plant J., 52, 405-419; Ruuska et al., 2002, Plant Cell, 14, 1191-1206; and Schwender and Ohlrogge, 2002, Plant Physiol. 130, 347-361). In green algae, glycolysis and pyruvate kinase (PK), which catalyze the irreversible synthesis of pyruvate from PEP, are present in the chloroplast in addition to the cytosol (for example, as described in Andre et al., 2007, Plant Cell, 19, 2006-2022). Therefore, it is possible that glycolysis-derived pyruvate is the major photosynthate to be converted to acetyl CoA for de novo fatty acid synthesis. An ACCase is generally considered to catalyze the first reaction of the fatty acid biosynthetic pathway—the formation of malonyl CoA from acetyl CoA and CO₂. This reaction takes place in two steps and is catalyzed by a single enzyme complex. In the first step, which is ATP-dependent, CO₂ (from HCO₃⁻) is transferred by the biotin carboxylase prosthetic group of ACCase to a nitrogen of a biotin prosthetic group attached to the ε-amino group of a lysine residue. In the second step, catalyzed by carboxyltransferase, the activated CO₂ is transferred from biotin to acetyl CoA to form malonyl CoA (for example, as described in Ohlrogge and Browse, 1995, Plant Cell, 7, 957-970).

According to Ohlrogge and Browse (1995, Plant Cell, 7, 957-970), malonyl CoA, the product of the carboxylation reaction, is the central carbon donor for fatty acid synthesis. The malonyl group is transferred from CoA to a protein co-factor on the acyl carrier protein (ACP; FIG. 2). All subsequent reactions of the pathway involve ACP until the finished products are ready for transfer to glycerolipids or export from the chloroplast. The malonyl group of malonyl ACP participates in a series of condensation reactions with acyl ACP (or acetyl CoA) acceptors. The first condensation reaction forms a four-carbon product, and is catalyzed by the condensing enzyme, 3-ketoacyl ACP synthase III (KAS III) (for example, as described in Jaworski et al., 1989, Plant Physiol., 90, 41-44). Another condensing enzyme, KAS I, is responsible for producing varying chain lengths (6-16 carbons). Three additional reactions occur after each condensation. To form a saturated fatty acid the 3-ketoacyl ACP product is reduced by the enzyme 3-ketoacyl ACP reductase, dehydrated by hydroxyacyl ACP dehydratase and then reduced by the enzyme enoyl ACP reductase (FIG. 2). These four reactions lead to a lengthening of the precursor fatty acid by two carbons. The fatty acid biosynthesis pathway produces saturated 16:0- and 18:0-ACP. To produce an unsaturated fatty acid, a double bond is introduced by the soluble enzyme stearoyl ACP desaturase. The elongation of fatty acids is terminated either when the acyl group is removed from ACP by an acyl-ACP thioesterase that hydrolyzes the acyl ACP and releases free fatty acid, or acyltransferases in the chloroplast transfer the fatty acid directly from ACP to glycerol-3-phosphate or monoacylglycerol-3-phosphate (for example, as described in Ohlrogge and Browse. 1995, Plant Cell, 7, 957-970). The final fatty acid composition of individual algae is determined by the activities of enzymes that use these acyl ACPs at the termination phase of fatty acid synthesis.

ACCases have been purified and kinetically characterized from two unicellular algae, the diatom Cyclotella cryptic (for example, as described in Roessler, 1990, Plant Physiol. 92, 73-78) and the prymnesiophyte Isochrysis galbana (for example, as described in Livne and Sukenik. 1990. Plant Cell Physiol. 31, 851-858). Native ACCase isolated from Cyclotella cryptica has a molecular mass of approximately 740 kDa, and appears to be composed of four identical biotin-containing subunits. The molecular mass of the native ACCase from I. galbana was estimated at 700 kDa. This suggests that ACCases from algae and the majority of ACCases from higher plants are similar in that they are composed of multiple identical subunits, each of which are multi-functional peptides containing domains responsible for both biotin carboxylation and subsequent carboxyl transfer to acetyl CoA (for example, as described in Roessler, 1990, Plant Physiol. 92, 73-78).

Roessler (1988, Arch. Biochem. Biophys. 267, 521-528) investigated changes in the activities of various lipid and carbohydrate biosynthetic enzymes in the diatom Cyclotella cryptica in response to silicon deficiency. The activity of ACCase increased approximately two and four fold after 4 hours and 15 hours of silicon-deficient growth, respectively, suggesting that the higher enzymatic activity may partially result from a covalent modification of the enzyme. As the increase in enzymatic activity can be blocked by the addition of protein synthesis inhibitors, it was suggested that the enhanced ACCase activity could also be the result of an increase in the rate of enzyme synthesis (for example, as described in Roessler, 1988, Arch. Biochem. Biophys. 267, 521-528; and Roessler et al., 1994, Ann. N. Y. Acad. Sci. 721, 250-256).

The gene that encodes ACCase in Cyclotella cryptica has been isolated and cloned (for example, as described in Roessler and Ohlrogge, 1993, J. Biol. Chem. 268, 19254-19259). The gene was shown to encode a polypeptide composed of 2089 amino acids, with a molecular mass of 230 kDa. The deduced amino acid sequence exhibited strong similarity to the sequences of animal and yeast ACCases in the biotin carboxylase and carboxyltransferase domains. Less sequence similarity was observed in the biotin carboxyl carrier protein domain, although the highly conserved Met-Lys-Met sequence of the biotin binding site was present. The N-terminus of the predicted ACCase sequence has characteristics of a signal sequence, indicating that the enzyme may be imported into chloroplasts via the endoplasmic reticulum.

Triacylglycerol Biosynthesis

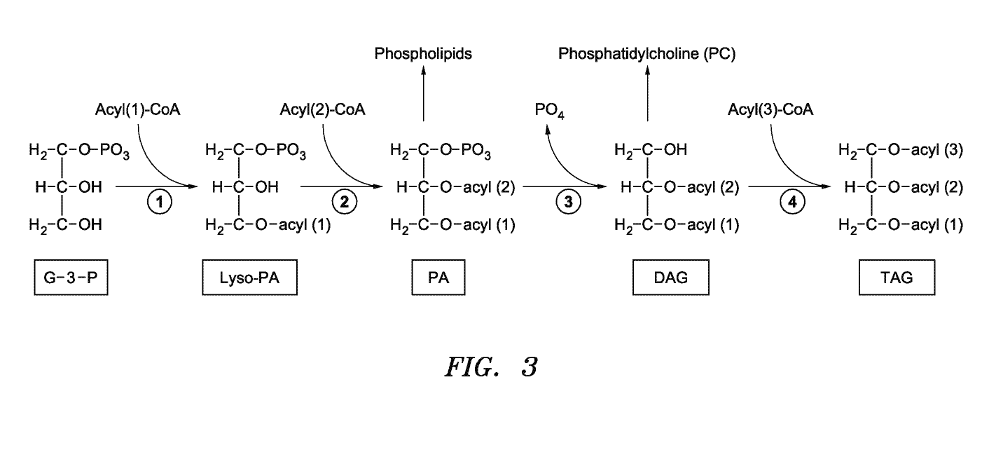

Triacylglycerol biosynthesis in algae has been proposed to occur via the direct glycerol pathway (FIG. 3) (for example, as described in Ratledge, 1988, An overview of microbial lipids. In Microbial Lipids, Vol. 1 (Ratledge, C. and Wilkerson. S. G., eds). New York: Academic Press, pp. 3-21). Fatty acids produced in the chloroplast are sequentially transferred from CoA to positions 1 and 2 of glycerol-3-phosphate, resulting in formation of the central metabolite phosphatidic acid (PA) (for example, as described in Ohlrogge and Browse, 1995, Plant Cell, 7, 957-970). Dephosphorylation of PA catalyzed by a specific phosphatase releases diacylglycerol (DAG). In the final step of TAG synthesis, a third fatty acid is transferred to the vacant position 3 of DAG, and this reaction is catalyzed by diacylglycerol acyltransferase, an enzymatic reaction that is unique to TAG biosynthesis. PA and DAG can also be used directly as a substrate for synthesis of polar lipids, such as phosphatidylcholine (PC) and galactolipids. The acyltransferases involved in TAG synthesis may exhibit preferences for specific acyl CoA molecules, and thus may play an important role in determining the final acyl composition of TAG. For example, Roessler et al. (1994, Genetic engineering approaches for enhanced production of biodiesel fuel from microalgae. In Enzymatic Conversion of Biomass for Fuels Production (Himmel, M. E., Baker, J. and Overend, R. P., eds). American Chemical Society, pp. 256-270)) reported that, in Nannochloropsis cells, the lyso-PA acyltransferase that acylates the second position (sn-2) of the glycerol backbone has a high substrate specificity, whereas glycerol-3-phosphate acyltransferase and DAG acyltransferase are less discriminating. It was also determined that lyso-PC acyltransferase prefers 18:1-CoA over 16:0-CoA.

Although the three sequential acyl transfers from acyl CoA to a glycerol backbone described above are believed to be the main pathway for TAG synthesis, Dahlqvist et al. (2000, Proc. Natl Acad. Sci. USA, 97, 6487-6492) reported an acyl CoA-independent mechanism for TAG synthesis in some plants and yeast. This pathway uses phospholipids as acyl donors and DAG as the acceptor, and the reaction is catalyzed by the enzyme phospholipid:diacylglycerol acyltransferase (PDAT). In an in vitro reaction system, the PDAT enzyme exhibited high substrate specificity for the ricinoleoyl or the vemoloyl group of PC, and it was suggested that PDAT could play an important role in the specific channeling of bilayer-disturbing fatty acids, such as ricinoleic and vernolic acids, from PC into the TAG pool (for example, as described in Dahlqvist et al., 2000. Proc. Natl Acad. Sci. USA, 97, 6487-6492). Under various stress conditions, algae usually undergo rapid degradation of the photosynthetic membrane with concomitant occurrence and accumulation of cytosolic TAG-enriched lipid bodies. If a PDAT orthologue were identified in an algal cell, especially in the chloroplast, then it is conceivable that that orthologue could use PC, PE or even galactolipids derived from the photosynthetic membrane as acyl donors in the synthesis of TAG. As such, the acyl CoA-independent synthesis of TAG could play an important role in the regulation of membrane lipid composition in response to various environmental and growth conditions, not only in plants and yeast but also in algae.

In most of the algal species/strains examined, TAGs are composed primarily of C14-C18 fatty acids that are saturated or mono-unsaturated (for example, as described in Harwood, 1998, Membrane lipids in algae. In Lipids in Photosynthesis: Structure, Function and Genetics (Siegenthaler, P. A. and Murata, N., eds). Dordrecht, The Netherlands: Kluwer Academic Publishers, pp. 53-64: and Roessler, 1990, J. Phycol. 26, 393-399). As exceptions, very-long-chain (>C20) PUFA synthesis and partitioning of such fatty acids into TAGs have been observed in the green alga Parietochloris incise (Trebouxiophyceae) (for example, as described in Bigogno et al., 2002, Phytochemistry, 60, 497-503), the freshwater red microalga Porphyridium cruentum (for example, as described in Cohen et al., 2000, Biochem. Soc. Trans. 28, 740-743), marine microalgae Nannochloropsis oculata (Eustigmatophyceae), P. tricornutum and Thalassiosira pseudonana (Bacillariophyceae), and the thraustochytrid Thraustochytrium aureum (for example, as described in Iida et al., 1996, J. Ferment. Bioeng. 81, 76-78). A strong positional preference of C22:6 in TAG for the sn-1 and sn-3 positions of the glycerol backbone was reported in the marine microalga Crypthecodinium cohnii (for example, as described in Kyle et al., 1992, Bioproduction of docoshexaenoic acid (DHA) by microalgae. In Industrial Applications of Single Cell Oils (Kyle, D. J. and Ratledge. C., eds). Champaign, Ill.: American Oil Chemists' Society, pp. 287-300). It has been proposed that very long PUFA-rich TAGs may occur as the result of ‘acyl shuttle’ between diacyl glycerol and/or TAG and phospholipid in situations where PUFAs are formed (for example, as described in Kamisaka et al., 1999, Biochim. Biophys. Acta, 1438, 185-198). The biosynthesis of very long PUFAs has been reviewed in detail elsewhere (for example, as described in Certik and Shimizu, 1999, J. Biosci. Bioeng. 87, 1-14; and Guschina and Harwood, 2006, Prog. Lipid Res. 45, 160-186).

Comparison of Lipid Metabolism in Algae and Higher Plants

Although algae generally share similar fatty acid and TAG synthetic pathways with higher plants, there is some evidence that differences in lipid metabolism do occur. In algae, for example, the complete pathway from carbon dioxide fixation to TAG synthesis and sequestration takes place within a single cell, whereas the synthesis and accumulation of TAG only occurs in special tissues or organs (e.g. seeds or fruits) of oil crop plants. In addition, very long PUFAs above C18 cannot be synthesized in significant amounts by naturally occurring higher plants, whereas many algae (especially marine species) have the ability to synthesize and accumulate large quantities of very long PUFAs, such as eicosapentaenoic acid (C20:5×3), docosahexaenoic acid (C22:6×3), and arachidonic acid (C20:4×6). Annotation of the genes involved in lipid metabolism in the green alga C. reinhardtii has revealed that algal lipid metabolism may be less complex than in Arabidopsis, and this is reflected in the presence and/or absence of certain pathways and the apparent sizes of the gene families that represent the various activities (for example, as described in Riekhof et al., 2005, Eukaryotic Cell, 4, 242-252).

Factors Affecting Triacylglycerol Accumulation and Fatty Acid Composition

Although the occurrence and the extent to which TAG is produced appear to be species/strain-specific, and are ultimately controlled by the genetic make-up of individual organisms, oleaginous algae produce only small quantities of TAG under optimal growth or favorable environmental conditions (for example, as described in Hu, 2004, Environmental effects on cell composition. In Handbook of Microalgal Culture (Richmond, A., ed.). Oxford: Blackwell, pp. 83-93). Synthesis and accumulation of large amounts of TAG accompanied by considerable alterations in lipid and fatty acid composition occur in the cell when oleaginous algae are placed under stress conditions imposed by chemical or physical environmental stimuli, either acting individually or in combination. The major chemical stimuli are nutrient starvation, salinity, and growth-medium pH. The major physical stimuli are temperature and light intensity. In addition to chemical and physical factors, growth phase and/or aging of the culture also affects TAG content and fatty acid composition.

Nutrients

Of all the nutrients evaluated, nitrogen limitation is the single most critical nutrient affecting lipid metabolism in algae. A general trend towards accumulation of lipids, particularly TAG, in response to nitrogen deficiency has been observed in numerous species or strains of various algal taxa, as shown in FIG. 1 (for example, as described in Basova. 2005. Int. J. Algae. 7, 33-57; Beijerinck, 1904, Rec. Trav. Bot. Neerl. 1, 28-40; Cobelas and Lechado, 1989, Grasas y Aceites, 40, 118-145; Merzlyak et al., 2007, J. Phycol. 43, 833-843; Roessler, 1990, J. Phycol. 26, 393-399; Shifrin and Chisholm, 1981, J. Phycol. 17, 374-384; Spoehr and Milner, 1949, Plant Physiol. 24, 120-149; and Thompson, 1996, Biochim. Biophys. Acta, 1302, 17-45).

In diatoms, silicon is an equally important nutrient that affects cellular lipid metabolism. For example, silicon-deficient Cyclotella cryptica cells have been shown to have higher levels of neutral lipids (primarily TAG) and higher proportions of saturated and mono-unsaturated fatty acids than silicon-replete cells (for example, as described in Roessler, 1988, Arch. Biochem. Biophys. 267, 521-528).

Other types of nutrient deficiency that promote lipid accumulation include phosphate limitation and sulfate limitation. For example, phosphorus limitation results in increased lipid content, mainly TAG, in Monodus subterraneus (Eustigmatophyceae) (for example, as described in Khozin-Goldberg and Cohen. 2006. Phytochemistry, 67, 696-701), P. tricornutum and Chaetoceros sp. (Bacillariophyceae), and I. galbana and Pavlova lutheri (Prymnesiophyceae), but decreased lipid content in Nannochloris atomus (Chlorophyceae) and Tetraselmis sp. (Prasinophyceae) (for example, as described in Reitan et al., 1994. J. Phycol. 30, 972-979). Of marine species examined (for example, as described in Reitan et al., 1994, J. Phycol. 30, 972-979), increased phosphorus deprivation was found to result in a higher relative content of 16:0 and 18:1, and a lower relative content of 18:4×3, 20:5×3, and 22:6×3. Studies have also shown that sulfur deprivation enhances the total lipid content in the green algae Chlorella sp. (for example, as described in Otsuka, 1961. J. Gen. Appl. Microbiol. 7, 72-77) and C. reinhardtii (for example, as described in Sato et al., 2000, Environmental effects on acidic lipids of thylakoid membranes. In Recent Advances in the Biochemistry of Plant Lipids (Harwood, J. L. and Quinn, P. J., eds). London: Portland Press Ltd, pp. 912-914).

Cyanobacteria appear to react to nutrient deficiency differently to eukaryotic algae. Piorreck and Pohl (1984, Phytochemistry, 23, 217-233) investigated the effects of nitrogen deprivation on the lipid metabolism of the cyanobacteria Anacystis nidulans, Microcystis aeruginosa, Oscillatoria rubescens and Spirulina platensis, and reported that either lipid content or fatty acid composition of these organisms was changed significantly under nitrogen-deprivation conditions. When changes in fatty acid composition occur in an individual species or strain in response to nutrient deficiency, the C18:2 fatty acid levels decreased, whereas those of both C16:0 and C18:1 fatty acids increased, similar to what occurs in eukaryotic algae (for example, as described in Olson and Ingram, 1975, J. Bacteriol. 124, 373-379). In some cases, nitrogen starvation resulted in reduced synthesis of lipids and fatty acids (for example, as described in Saha et al., 2003, FEMS Microbiol. Ecol. 45, 263-272).

Temperature

Temperature has been found to have a major effect on the fatty acid composition of algae. A general trend towards increasing fatty acid unsaturation with decreasing temperature and increasing saturated fatty acids with increasing temperature has been observed in many algae and cyanobacteria (for example, as described in Lynch and Thompson, 1982, Plant Physiol. 69, 1369-1375; Murata et al., 1975, Plant Physiol. 56, 508-517; Raison, 1986, Alterations in the physical properties and thermal responses of membrane lipids: correlations with acclimation to chilling and high temperature. In Frontiers of Membrane Research in Agriculture (St John, J. B., Berlin, E. and Jackson, P. G., eds) Totowa, N.J.: Rowman and Allanheld, pp. 383-401; Renaud et al., 2002. Aquaculture, 211, 195-214; and Sato and Murata. 1980. Biochim. Biophys. Acta. 619, 353-366). It has been generally speculated that the ability of algae to alter the physical properties and thermal responses of membrane lipids represents a strategy for enhancing physiological acclimatization over a range of temperatures, although the underlying regulatory mechanism is unknown (for example, as discussed in Somerville. 1995. Proc. Natl Acad. Sci. USA, 92, 6215-6218). Temperature also affects the total lipid content in algae. For example, the lipid content in the chrysophytan Ochromonas danica (for example, as described in Aaronson, 1973, J. Phycol. 9, 111-113) and the eustigmatophyte Nannochloropsis salina (for example, as described in Boussiba et al., 1987, Biomass, 12, 37-47) increases with increasing temperature. In contrast, no significant change in the lipid content was observed in Chlorella sorokiniana grown at various temperatures (for example, as described in Patterson, 1970, Lipids, 5, 597-600).

Light Intensity

Algae grown at various light intensities exhibit remarkable changes in their gross chemical composition, pigment content and photosynthetic activity (for example, as described in Falkowski and Owens. 1980. Plant Physiol. 66, 592-595; Post et al., 1985, Mar. Ecol. Prog. Series, 25, 141-149; Richardson et al., 1983, New Phytol. 93, 157-191; and Sukenik et al., 1987, Nature, 327, 704-707). Typically, low light intensity induces the formation of polar lipids, particularly the membrane polar lipids associated with the chloroplast, whereas high light intensity decreases total polar lipid content with a concomitant increase in the amount of neutral storage lipids, mainly TAGs (for example, as described in Brown et al., 1996, J. Phycol. 32, 64-73; Khotimchenko and Yakovleva, 2005, Phytochemistry, 66, 73-79; Napolitano, 1994, J. Phycol. 30, 943-950: Orcutt and Patterson, 1974, Lipids, 9, 1000-1003; Spoehr and Milner, 1949, Plant Physiol. 24, 120-149; and Sukenik et al., 1989, J. Phycol. 25, 686-692).

The degree of fatty acid saturation can also be altered by light intensity. In Nannochloropsis sp., for example, the percentage of the major PUFA C20:5×3 remained fairly stable (approximately 35% of the total fatty acids) under light-limited conditions. However, it decreased approximately threefold under light-saturated conditions, concomitant with an increase in the proportion of saturated and mono-unsaturated fatty acids (i.e. C14, C16:0 and C16:1×7) (Fabregas et al., 2004). Based upon the algal species/strains examined (for example, as described in Orcutt and Patterson, 1974, Lipids, 9, 1000-1003; and Sukenik et al., 1993, J. Phycol. 29, 620-626), it appears, with a few exceptions, that low light favors the formation of PUFAs, which in turn are incorporated into membrane structures. On the other hand, high light alters fatty acid synthesis to produce more of the saturated and mono-unsaturated fatty acids that mainly make up neutral lipids.

Growth Phase and Physiological Status

Lipid content and fatty acid composition are also subject to variability during the growth cycle. In many algal species examined, an increase in TAGs is often observed during stationary phase. For example, in the chlorophyte Parietochloris incise, TAGs increased from 43% (total fatty acids) in the logarithmic phase to 77% in the stationary phase (for example, as described in Bigogno et al., 2002, Phytochemistry, 60, 497-503), and in the marine dinoflagellate Gymnodinium sp., the proportion of TAGs increased from 8% during the logarithmic growth phase to 30% during the stationary phase (for example, as described in Mansour et al., 2003, Phytochemistry, 63, 145-153). Coincident increases in the relative proportions of both saturated and mono-unsaturated 16:0 and 18:1 fatty acids and decreases in the proportion of PUFAs in total lipid were also associated with growth-phase transition from the logarithmic to the stationary phase. In contrast to these decreases in PUFAs, however, the PUFA arachidonic acid (C20:4×6) is the major constituent of TAG produced in Parietochloris incise cells (for example, as described in Bigogno et al., 2002, Phytochemistry. 60, 497-503), while docosahexaenoic acid (22:6×3) and eicosapentacnoic acid (20:5×3) are partitioned to TAG in the Eustigmatophyceac N. oculata, the diatoms P. tricornutum and T. pseudonana, and the haptophyte Pavlova lutheri (for example, as described in Tonon et al., 2002, Phytochemistry 61, 15-24).

Culture aging or senescence also affects lipid and fatty acid content and composition. The total lipid content of cells increased with age in the green alga Chlorococcum macrostigma (for example, as described in Collins and Kalnins, 1969. Phyton, 26, 47-50), and the diatoms Nitzschia palea (for example, as described in von Denffer, 1949, Arch. Mikrobiol. 14, 159-202). Thalassiosira fluviatillis (for example, as described in Conover, 1975, Mar. Biol. 32, 231-246) and Coscinodiscus eccentricus (for example, as described in Pugh, 1971, Mar. Biol. 11, 118-124). An exception to this was reported in the diatom P. tricornutum, where culture age had almost no influence on the total fatty acid content, although TAGs were accumulated and the polar lipid content was reduced (for example, as described in Alonso et al., 2000, Phytochemistry. 54, 461-471). Analysis of fatty acid composition in the diatoms P. tricornutum and Chaetoceros muelleri revealed a marked increase in the levels of saturated and monounsaturated fatty acids (e.g. 16:0, 16:1×7 and 18:1×9), with a concomitant decrease in the levels of PUFAs (e.g. 16:3×4 and 20:5×3) with increasing culture age (for example, as described in Liang et al., 2006. Bot. Mar. 49, 165-173). Most studies on algal lipid metabolism have been carried out in a batch culture mode. Therefore, the age of a given culture may or may not be associated with nutrient depletion, making it difficult to separate true aging effects from nutrient deficiency-induced effects on lipid metabolism.

Physiological Roles of Triacylglycerol Accumulation

Synthesis of TAG and deposition of TAG into cytosolic lipid bodies may be, with few exceptions, the default pathway in algae under environmental stress conditions. In addition to the obvious physiological role of TAG serving as carbon and energy storage, particularly in aged algal cells or under stress, the TAG synthesis pathway may play more active and diverse roles in the stress response. The de novo TAG synthesis pathway serves as an electron sink under photo-oxidative stress. Under stress, excess electrons that accumulate in the photosynthetic electron transport chain may induce over-production of reactive oxygen species, which may in turn cause inhibition of photosynthesis and damage to membrane lipids, proteins and other macromolecules. The formation of a C18 fatty acid consumes approximately 24 NADPH derived from the electron transport chain, which is twice that required for synthesis of a carbohydrate or protein molecule of the same mass, and thus relaxes the over reduced electron transport chain under high light or other stress conditions. The TAG synthesis pathway is usually coordinated with secondary carotenoid synthesis in algae (for example, as described in Rabbani et al., 1998, Plant Physiol. 116, 1239-1248; and Zhekisheva et al., 2002, J. Phycol. 38, 325-331). The molecules (e.g. b-carotene, lutein or astaxanthin) produced in the carotenoid pathway are esterified with TAG and sequestered into cytosolic lipid bodies. The peripheral distribution of carotenoid-rich lipid bodies serve as a ‘sunscreen’ to prevent or reduce excess light striking the chloroplast under stress. TAG synthesis may also utilize PC, PE, and galactolipids or toxic fatty acids excluded from the membrane system as acyl donors, thereby serving as a mechanism to detoxify membrane lipids and deposit them in the form of TAG.

Role of Algal Genomics and Model Systems in Biofuel Production

Because of the potential for photosynthetic micro-organisms to produce 8-24 times more lipids per unit area for biofuel production than the best land plants (for example, as described in Sheehan et al., 1998. A Look Back at the US Department of Energy's Aquatic Species Program—Biodiesel from Algae, Close Out Report TP-580-24190. Golden. Colo.: National Renewable Energy Laboratory), these microbes are in the forefront as future biodiesel producers. Cyanobacteria, for which over 20 completed genome sequences are available (http://genome.jgi-psf.org/mic_cur1.html) (over 30 are in progress), produce some lipids. In addition, the nuclear genomes of eight microalgae, some of which can produce significant quantities of storage lipids, have also been sequenced (http://genome.jgipsf.org/euk_cur1.html). These eukaryotes include C. reinhardtii (Plant Physiol. (2003) Vol. 131, pp. 401-408), Volvox carteri (green alga)(BMC Genomics (2009) 10:132), Cyanidioschizon merolae (red alga) (DNA Research (2003) 10(2):67-77). Osteococcus lucimarinus (Proc Natl Acad Sci U.S.A. (2007) 104, 7705-7710). Osteococcus tauris (marine pico-eukaryotes)(Trends in Genetics, Vol. 23, Issue 4 (2007) pp. 151-154), Aureococcus annophageferrens (a harmful algal bloom component; http://genome.jgi-psf.org/Auran1/Auran1.info.html; sequence not yet published). P. tricornutum (Nature (2008) 456(7219):239-44; and Plant Physiol. (2002) Vol. 129, p. 993-1002), and T. pseudonana (diatoms) (Nature (2008) 456 (7219):239-44; and Science (2004) October 1; 306:5693).

Chlamydomonas reinhardtii is a single celled chlorophyte. Highly adaptable, these green algae live in many different environments throughout the world. Normally deriving energy from photosynthesis, with an alternative carbon source, C. reinhardtii can also thrive in total darkness.

The relative adaptability and quick generation time has made Chlamydomonas an important model for biological research. The C. reinhardtii genome is described in Science (2007) 318(5848):245-50.

Volvox carteri is a multicellular chlorophyte alga, closely related to the single-celled Chlamydomonas reinhardtii. Volvox normally reproduces as an asexual haploid, but can be induced to undergo sexual differentiation and reproduction. The 48-hour life cycle allows easy laboratory culture and includes an embryogenesis program that features many of the hallmarks of animal and plant development. These features include embryonic axis formation, asymmetric cell division, a gastrulation-like inversion, and differentiation of germ and somatic cells. The ˜2000 somatic cells in a Volvox spheroid are biflagellate and adapted for motility, while the ˜16 large germ cells contained within the spheroid are non-motile and specialized for growth and reproduction. Volvox embryogensis generates the coordinated arrangement of somatic flagella and photosensing eye spots needed for the organism's characteristic forward rolling motion. The Volvocales family includes single-celled Chlamydomonas (whose genome sequence is available) and Volvox, also includes several multicellular or colonial species with intermediate cell numbers and less complex developmental programming.

Ostreococcus belongs to the Prasinophyceae, an early-diverging class within the green plant lineage, and is reported as a globally abundant, single-celled alga thriving in the upper (illuminated) water column of the oceans. The most striking feature of O. lucimarinus and related species is their minimal cellular organization: a naked, nearly 1-micron cell, lacking flagella, with a single chloroplast and mitochondrion. The Ostreococcus genome is described in Proc Natl Acad Sci U.S.A. (2007) 104, 7705-7710.

Three different ecotypes or potential species have been defined, based on their adaptation to light intensity. One (O. lucimarinus) is adapted to high light intensities and corresponds to surface-isolated strains. The second (RCC141) has been defined as low-light and includes strains from deeper in the water column. The third (O. tauri) corresponds to strains isolated from a coastal lagoon and can be considered light-polyvalent. Comparative analysis of Ostreococcus sp will help to understand niche differentiation in unicellular eukaryotes and evolution of genome size in eukaryotes.

Aureococcus anophageffrens is a 2-3 um spherical, non-motile pelagophyte which has caused destructive ‘brown tide’ blooms in northeast and mid-Atlantic US estuaries for two decades. A coastal microalgae species, A. anophagefferens is capable of growing to extremely high densities (>10E9 cells L-1) and can enzymatically degrade complex forms of dissolved organic matter as a source of cellular carbon and nitrogen. This species is also known to be well adapted to low light, is associated with annually elevated water temperatures, can rapidly reduce trace metals, and sequesters substantial amounts of carbon during bloom events. The Aureococcus is a Harmful Algal Bloom (HAB) species. HABs are blooms of phytoplankton cells resulting in conditions that are unhealthy for humans, animals or ecosystems causing by decrease in light attenuation or oxygen levels, or by production of toxins. HABs may cause marine life poisoning and/or death.

P. tricornutum and T. pseudononan are both diatoms. Diatoms are eukaryotic, photosynthetic microorganisms found throughout marine and freshwater ecosystems that are responsible for around 20% of global primary productivity. A defining feature of diatoms is their ornately patterned silicified cell wall (known as frustule), which display species-specific nanoscale-structures. These organisms therefore play major roles in global carbon and silicon cycles.

The marine pennate diatom Phaeodactylum tricornutum is the second diatom for which a whole genome sequence has been generated. It was chosen primarily because of the superior genetic resources available for this diatom (eg, genetic transformation, 100,000 ESTs), and because it has been used in laboratory-based studies of diatom physiology for several decades. Although not considered to be of great ecological significance, it has been found in several locations around the world, typically in coastal areas with wide fluctuations in salinity. Unlike other diatoms it can exist in different morphotypes, and changes in cell shape can be stimulated by environmental conditions. This feature can be used to explore the molecular basis of cell shape control and morphogenesis. Furthermore the species can grow in the absence of silicon, and the biogenesis of silicified frustules is facultative, thereby providing opportunities for experimental exploration of silicon-based nanofabrication in diatoms. The sequence is 30 mega base pairs and, together with the sequence from the centric diatom Thalassiosira pseudonana (34 Mbp; the first diatom whole genome sequence), it provides the basis for comparative genomics studies of diatoms with other eukaryotes and will provide a foundation for interpreting the ecological success of these organisms.

The clone of P. tricormutum that was sequenced is CCAP1055/1 and is available from the Culture Collection of Algae and Protozoa (CCAP). This clone represents a monoclonal culture derived from a fusiform cell in May 2003 from strain CCMP632, which was originally isolated in 1956 off Blackpool (U.K.). It has been maintained in culture continuously in F/2 medium. The Phaeodactylum genome is described in Nature (2008) 456(7219):239-44.

Extensive genomic, biological and physiological data exist for C. reinhardtii, a unicellular, water-oxidizing green alga (for example, as described in Grossman, 2005, Plant Physiol. 137, 410-427; Merchant et al., 2007, Science, 318, 245-251; and Mus et al., 2007, J. Biol. Chem. 282, 25475-25486). For these reasons, Chlamydomonas has emerged recently as a model eukaryote microbe for the study of many processes, including photosynthesis, phototaxis, flagellar function, nutrient acquisition, and the biosynthesis and functions of lipids.

The recent availability of the Chlamydomonas genome sequence and biochemical studies indicate that this versatile, genetically malleable eukaryote has an extensive network of diverse metabolic pathways that are unprecedented in other eukaryotes for which whole-genome sequence information is available. Chlamydomonas is of particular interest to renewable energy efforts because its metabolism can be manipulated by nutrient stress to accumulate various energy-yielding reduced compounds.

The advantage of C. reinhardtii as a model for oxygenic photosynthesis derives mainly from its ability to grow either photo-, mixo- or heterotrophically (in the dark and in the presence of acetate) while maintaining an intact, functional photosynthetic apparatus. This property has allowed researchers to study photosynthetic mutations that are lethal in other organisms. Moreover, C. reinhardtii spends most of its life cycle as a haploid organism of either mating type + or) (Harris, 1989, The Chlamydomonas Sourcebook. A Comprehensive Guide to Biology and Laboratory Use. San Diego, Calif.: Academic Press). Gametogenesis is triggered by environmental stresses, particularly nitrogen deprivation (Sager and Granick, 1954, J. Gen. Physiol. 37, 729-742), and its occurrence can be synchronized by light/dark periods of growth (Kates and Jones, 1964, Biochim. Biophys. Acta, 86, 438-447). During its haploid stage, C. reinhardtii can be genetically engineered and single genotypes easily generated. Additionally, different phenotypes can be obtained by crossing two haploid mutants of different mating types carrying different genotypes. Conversely, single-mutant genotypes can be unveiled by back-crossing mutants carrying multiple mutations with the wild-type strain of the opposite mating type.

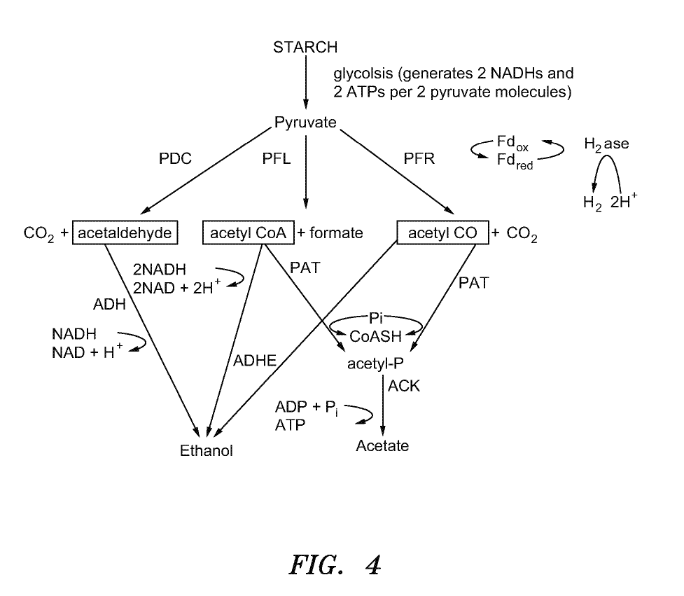

Chlamydomonas reinhardtii can also be used as a model organism for fermentation, given the number of pathways identified under anaerobic conditions biochemically (for example, as described in Gfeller and Gibbs, 1984, Plant Physiol. 75, 212-218; and Ohta et al., 1987, Plant Physiol. 83, 1022-1026) or by microarray analysis (for example, as described in Mus et al., 2007, J. Biol. Chem. 282, 25475-25486). The results, summarized in FIG. 4, suggest that both the pyruvate formate lyase (PFL) and the pyruvate ferredoxin oxidoreductase (PFR) pathways are functional in C. reinhardtii under anaerobiosis, as well as the pyruvate decarboxylase (PDC) pathway. The former two pathways generate acetyl CoA (a precursor for lipid metabolism) and either formate (PFL) or H2 (PFR), and the latter can generate ethanol through the alcohol dehydrogenase (ADH)-catalyzed reduction of acetaldehyde. Finally, acetyl CoA can be further metabolized by C. reinhardtii to ethanol, through the alcohol/aldehyde bifunctional dehydrogenase (ADHE) activity, or to acetate, through the sequential activity of two enzymes, phosphotransacetylase (PAT) and acetate kinase (ACK). The last reaction releases ATP. Mus et al. (2007, J. Biol. Chem. 282, 25475-25486) and Hemschemeier and Happe (2005, Chem. Soc. Trans. 33, 39-41) proposed that the unprecedented presence of all these pathways endows C. reinhardtii with a higher flexibility to adapt to environmental conditions. Finally, fermentative lactate production has been detected under certain conditions (Kreuzberg, 1984, Physiol. Plant, 61, 87-94).

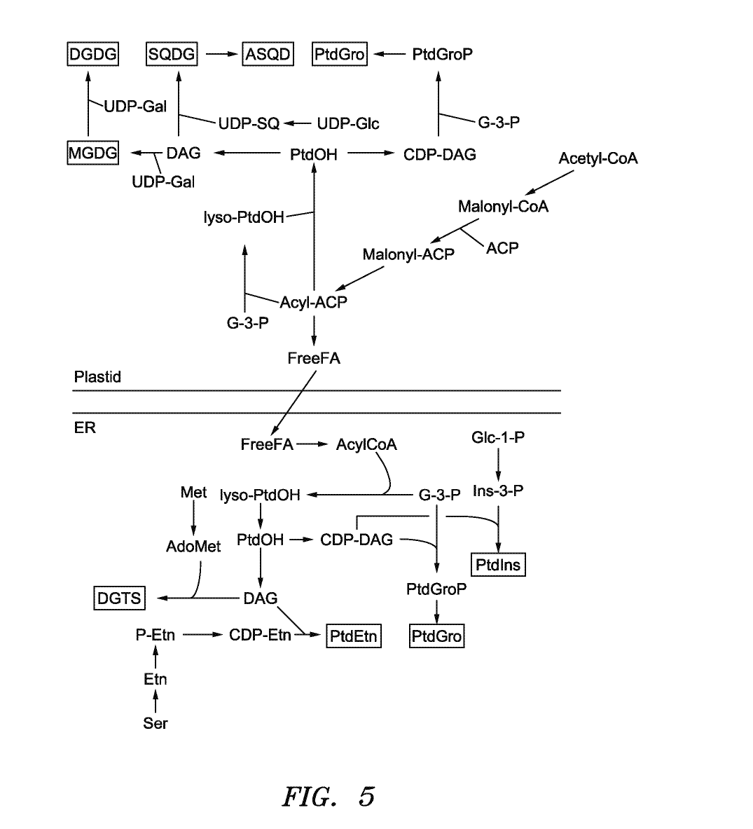

Although pathways for fatty acid biosynthesis are present in C. reinhardtii (FIG. 5), they are not known to be over expressed under normal photo-autotrophic or mixotrophic growth (for example, as described in Harris, 1989, The Chlamydomonas Sourcebook. A Comprehensive Guide to Biology and Laboratory Use. San Diego, Calif.: Academic Press). However, these pathways could be artificially over-expressed in C. reinhardtii.

Global expression profiling of Chlamydomonas under conditions that produce biofuels (H2 in this case) (for example, as described in Mus et al., 2007, J. Biol. Chem. 282, 25475-25486) has been reported using second-generation microarrays with 10,000 genes of the over 15,000 genes predicted (for example, as described in Eberhard et al., 2006, Curr. Genet. 49, 106-124; and Merchant et al., 2007, Science, 318, 245-251). However, much of the information that was reported involves fermentative metabolism, as discussed above. Little or no research has been conducted to characterize the up- and down regulation of genes associated with lipid metabolism when Chlamydomonas is exposed to nutrient stress. N-deprived C. reinhardtii will over-accumulate starch and lipids that can be used for formate, alcohol and biodiesel production (for example, as described in Mus et al., 2007, J. Biol. Chem. 282, 25475-25486; and Riekhof et al., 2005, Eukaryotic Cell, 4, 242-252).

Other organisms, for example, those listed in the “Host Cells or Host Organisms” section of the disclosure can be used as a system for the production of useful products, for example, fatty acids, glycerol lipids or biofuels.

Lipid Accumulation by Microalgae.

Under certain growth conditions, many microalgae can produce lipids that are suitable for conversion to liquid transportation fuels. In the late 1940s, nitrogen limitation was reported to significantly influence microalga lipid storage. Spoehr and Milner (1949, Plant Physiol. 24, 120-149) published detailed information on the effects of environmental conditions on algal composition, and described the effect of varying nitrogen supply on the lipid and chlorophyll content of Chlorella and some diatoms. Investigations by Collyer and Fogg (1955, J. Exp. Bot. 6, 256-275) demonstrated that the fatty acid content of most green algae was between 10 and 30% DCW. Werner (1966, Arch. Mikrobiol. 55, 278-308) reported an increase in the cellular lipids of a diatom during silicon starvation. Coombs et al. (1967, Plant Physiol. 42, 1601-1606) reported that the lipid content of the diatom Navicula pelliculosa increased by about 60% during a 14 h silicon starvation period. In addition to nutrition, fatty acid and lipid composition and content were also found to be influenced by a number of other factors such as light (for example, as described in Constantopolous and Bloch, 1967, J. Biol. Chem. 242, 3538-3542; Nichols, 1965, Biochim. Biophys. Acta, 106, 274-279; Pohl and Wagner, 1972, Z. Naturforsch. 27, 53-61; and Rosenberg and Gouaux, 1967, J. Lipid Res. 8, 80-83) and low temperatures (for example, as described in Ackman et al., 1968, J. Fisheries Res. Board Canada. 25, 1603-1620).

Microalgal Physiology and Biochemistry.

Studies on algal physiology under the Aquatic Species Program (ASP) centered on the ability of many species to induce lipid biosynthesis under conditions of nutrient stress (for example, as described in Dempster and Sommerfeld, 1998, J. Phycol. 34, 712-721; and McGinnis et al., 1997, J. Appl. Phycol. 9, 19-24). Focusing on the diatom Cyclotella cryptica, biochemical studies indicated that silicon deficiency led to increased activity of the enzyme ACCase, which catalyzes the conversion of acetyl CoA to malonyl CoA, the substrate for fatty acid synthase (Roessler, 1988, Arch. Biochem. Biophys. 267, 521-528). The ACCase enzyme was extensively characterized (Roessler. 1990. Plant Physiol. 92, 73-78). Additional studies focused on the pathway for production of the storage carbohydrate chrysolaminarin, which is hypothesized to compete with the lipid pathway for fixed carbon. UDPglucose pyrophosphorylase (UGPase) and chrysolaminarin synthase activities from Cyclotella cryptica were also characterized (for example, as described in Roessler, 1987, J. Phycol. 23, 494-498; and 1988, Arch. Biochem. Biophys. 267, 521-528).

Microalgal Molecular Biology and Genetic Engineering.

In the latter years of the ASP, the research at the National Renewable Research Laboratory focused on the genetic engineering of green algae and diatoms for enhanced lipid production. Genetic transformation of microalgae was a major barrier to overcome. The first successful transformation of a microalga strain with potential for biodiesel production was achieved in 1994, with successful transformation of the diatoms Cyclotella cryptica and Navicula saprophila (Dunahay et al., 1995, J. Phycol. 31, 1004-1012). The technique utilized particle bombardment and an antibiotic resistance selectable marker under the control of the ACCase promoter and terminator elements. The second major accomplishment was the isolation and characterization of genes from Cyclotella cryptica that encoded the ACCase and UGPase enzymes (Jarvis and Roessler. 1999. U.S. Pat. No. 5,928,932; Roessler and Ohlrogge, 1993, J. Biol. Chem. 268, 19254-19259). Attempts to alter the expression level of the ACCase and UGPase genes in Cyclotella cryptica using this transformation system met with some success, but effects on lipid production were not observed in these preliminary experiments (Sheehan et al., 1998, US Department of Energy's Office of Fuels Development, July 1998. A Look Back at the US Department of Energy's Aquatic Species Program—Biodiesel from Algae, Close Out Report TP-580-24190. Golden, Colo.: National Renewable Energy Laboratory).

New tag-sequencing methodologies such as 454 (Roche, USA) and Solexa (Illumina, USA), can give an accurate whole-genome picture of expression data, and can be used to provide a quantitative picture of the mRNAs in algal samples.

Procedures for metabolite profiling of C. reinhardtii CC-125 cells, which quickly inactivate enzymatic activity, optimize extraction capacity, and are amenable to large sample sizes, were reported by Bolling and Fiehn, (2005, Plant Physiol. 139, 1995-2005). The study explored profiles of Tris-acetate/phosphate-grown cells as well as cells that were deprived of sulfate. Nitrogen-, phosphate- and iron-deprivation profiles were also examined, and each metabolic profile was different. Sulfur depletion leads to the anaerobic conditions required for induction of the hydrogenase enzyme and H2 production (for example, as described in Ghirardi et al., 2007, Annu. Rev. Plant Biol. 58, 71-91; and Hemschemeier et al., 2008. Planta, 227, 397-407). Rapidly sampled cells (cell leakage controls were determined by 14C-labeling techniques) were analyzed by gas chromatography coupled to time-of-flight mass spectrometry, and more than 100 metabolites (e.g. amino acids, carbohydrates, phosphorylated intermediates, nucleotides and organic acids) out of about 800 detected could be identified. The concentrations of a number of phosphorylated glycolysis intermediates increase significantly during sulfur stress (for example, as described in Bolling and Fiehn, 2005, Plant Physiol. 139, 1995-2005), consistent with the upregulation of many genes associated with starch degradation and fermentation observed in anaerobic Chlamydomonas cells (for example, as described in Mus et al., 2007, J. Biol. Chem. 282, 25475-25486). Lipid metabolism was not studied.

There are a number of relevant studies of Chlamydomonas proteomics, as reviewed by Stauber and Hippler (2004, Plant Physiol. Biochem. 42, 989-1001). However, no proteomics research has yet been reported in algae under biofuel-producing conditions.

Host Cells or Host Organisms

Biomass containing fatty acids and/or glycerol lipids that is useful in the methods and systems described herein can be obtained from host cells or host organisms.

A host cell can contain a polynucleotide encoding an SN protein of the present disclosure. In some embodiments, a host cell is part of a multicellular organism. In other embodiments, a host cell is cultured as a unicellular organism.

Host organisms can include any suitable host, for example, a microorganism. Microorganisms which are useful for the methods described herein include, for example, photosynthetic bacteria (e.g., cyanobacteria), non-photosynthetic bacteria (e.g., E. coli), yeast (e.g., Saccharomyces cerevisiae), and algae (e. g., microalgae such as Chlamydomonas reinhardtii).

Examples of host organisms that can be transformed with a polynucleotide of interest (for example, a polynucleotide that encodes for an SN protein) include vascular and non-vascular organisms. The organism can be prokaryotic or eukaryotic. The organism can be unicellular or multicellular. A host organism is an organism comprising a host cell. In other embodiments, the host organism is photosynthetic. A photosynthetic organism is one that naturally photosynthesizes (e.g., an alga) or that is genetically engineered or otherwise modified to be photosynthetic. In some instances, a photosynthetic organism may be transformed with a construct or vector of the disclosure which renders all or part of the photosynthetic apparatus inoperable.

By way of example, a non-vascular photosynthetic microalga species (for example, C. reinhardtii, Nannochloropsis oceania, N. salina, D. salina. H. pluvalis, S. dimorphus, D. viridis, Chlorella sp., and D. tertiolecta) can be genetically engineered to produce a polypeptide of interest, for example an SN protein. Production of the protein in these microalgae can be achieved by engineering the microalgae to express the protein in the algal chloroplast or nucleus.

In other embodiments the host organism is a vascular plant. Non-limiting examples of such plants include various monocots and dicots, including high oil seed plants such as high oil seed Brassica (e.g., Brassica nigra, Brassica napus, Brassica hirta, Brassica rapa, Brassica campestris, Brassica carinalta, and Brassica juncea), soybean (Glycine max), castor bean (Ricinus commmis), cotton, safflower (Carthamus tinctorius), sunflower (Helianthus annuus), flax (Linum usitatissimum), corn (Zea mays), coconut (Cocos nucifera, palm (Elaeis guineensis), oil nut trees such as olive (Olea europaea), sesame, and peanut (Arachis hypogaea), as well as Arabidopsis, tobacco, wheat, barley, oats, amaranth, potato, rice, tomato, and legumes (e.g., peas, beans, lentils, alfalfa, etc.).

The host organism or cell can be prokaryotic. Examples of some prokaryotic organisms of the present disclosure include, but are not limited to, cyanobacteria (e.g., Synechococcus, Synechocystis, Athrospira, Gleocapsa, Spirulina, Leptolyngbya, Lyngbya, Oscillatoria, and, Pseudoanabaena). Suitable prokaryotic cells include, but are not limited to, any of a variety of laboratory strains of Escherichia coli, Lactobacillus sp., Salmonella sp., and Shigella sp. (for example, as described in Carrier et al. (1992) J. Immunol. 148:1176-1181, U.S. Pat. No. 6,447,784; and Sizemore et al. (1995) Science 270:299-302). Examples of Salmonella strains which can be employed in the present disclosure include, but are not limited to, Salmonella typhi and S. typhimurium. Suitable Shigella strains include, but are not limited to, Shigella flexneri, Shigella sonnei, and Shigella disenteriae. Typically, the laboratory strain is one that is non-pathogenic. Non-limiting examples of other suitable bacteria include, but are not limited to, Pseudomonas pudita, Pseudomonas aeruginosa, Pseudomonas mevalonii, Rhodobacter sphaeroides, Rhodobacter capsulatus, Rhodospirillum rubrum, and Rhodococcus sp.

In some embodiments, the host organism or cell is eukaryotic (e.g. green algae, red algae, brown algae). In some embodiments, the alga is a green algae, for example, a Chlorophycean. The algae can be unicellular or multicellular. Suitable eukaryotic host cells include, but are not limited to, yeast cells, insect cells, plant cells, fungal cells, and algal cells. Suitable eukaryotic host cells include, but are not limited to, Pichia pastoris, Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis. Pichia methanolica, Pichia sp., Saccharomyces cerevisiae, Saccharomyces sp., Hansenula polymorpha, Kluyveromyces sp., Kluyveromyces lactis. Candida albicans, Aspergillus nidulans, Aspergillus niger, Aspergillus oryzae, Trichoderma reesci, Chrysosporium lucknowense, Fusarium sp., Fusarium gramineum, Fusarium venenatum, Neurospora crassa, and Chlamydomonas reinhardtii.

In some embodiments, eukaryotic microalgae, such as for example, a Chlamydomonas, Volvacales, Dunaliella, Nannochloropsis, Desmid, Desmodesmus, Scenedesmus, Volvox, Chlorella, Arthrospira, Sprirulina, Botryococcus, Desmodesmus, or Hematococcus species, can be used in the disclosed methods.

In other embodiments, the host cell is Chlamydomonas reinhardtii, Dunaliella salina, Haematococcus pluvialis, Nannochloropsis oceania, Nannochloropsis salina, Scenedesmus dimorphus, a Chlorella species, a Spirulina species, a Desmid species. Spirulina maximus, Arthrospira fusiformis, Dunaliella viridis, N. oculata, S. maximus, A. Fusiformis, or Dunaliella tertolecta.

In some instances the organism is a rhodophyte, chlorophyte, heterokontophyte, tribophyte, glaucophyte, chlorarachniophyte, euglenoid, haptophyte, cryptomonad, dinoflagellum, or phytoplankton.

In some instances a host organism is vascular and photosynthetic. Examples of vascular plants include, but are not limited to, angiosperms, gymnosperms, rhyniophytes, or other tracheophytes.

In some instances a host organism is non-vascular and photosynthetic. As used herein, the term “non-vascular photosynthetic organism,” refers to any macroscopic or microscopic organism, including, but not limited to, algae, cyanobacteria and photosynthetic bacteria, which does not have a vascular system such as that found in vascular plants.

Examples of non-vascular photosynthetic organisms include bryophtyes, such as marchantiophytes or anthocerotophytes.

In some instances the organism is a cyanobacteria. In some instances, the organism is algae (e.g., macroalgae or microalgae). The algae can be unicellular or multicellular algae. For example, the microalgae Chlamydomonas reinhardtii may be transformed with a vector, or a linearized portion thereof, encoding one or more proteins of interest (e.g., an SN protein).

Methods for algal transformation are described in U.S. Provisional Patent Application No. 60/142,091. The methods of the present disclosure can be carried out using algae, for example, the microalga, C. reinhardtii. The use of microalgae to express a polypeptide according to a method of the disclosure provides the advantage that large populations of the microalgae can be grown, including commercially (Cyanotech Corp.: Kailua-Kona Hi.), thus allowing for production and, if desired, isolation of large amounts of a desired product.

The vectors of the present disclosure may be capable of stable or transient transformation of multiple photosynthetic organisms, including, but not limited to, photosynthetic bacteria (including cyanobacteria), cyanophyta, prochlorophyta, rhodophyta, chlorophyta, heterokontophyta, tribophyta, glaucophyta, chlorarachniophytes, euglenophyta, euglenoids, haptophyta, chrysophyta, cryptophyta, cryptomonads, dinophyta, dinoflagellata, pyrmnesiophyta, bacillariophyta, xanthophyta, eustignmatophyta, raphidophyta, phaeophyta, and phytoplankton. Other vectors of the present disclosure are capable of stable or transient transformation of, for example, C. reinhardtii, N. oceania, N. salina, D. salina, H. pluvalis, S. dimorphus, D. viridis, or D. tertiolecta.

Examples of appropriate hosts, include but are not limited to: bacterial cells, such as E. coli, Streptomyces, Salmonella typhimurium; fungal cells, such as yeast; insect cells, such as Drosophila S2 and Spodoptera Sf9; animal cells, such as CHO, COS or Bowes melanoma: adenovinises; and plant cells. The selection of an appropriate host is deemed to be within the scope of those skilled in the art.

A polynucleotide selected and isolated as described herein is introduced into a suitable host cell. A suitable host cell is any cell which is capable of promoting recombination and/or reductive reassortment. The selected polynucleotides can be, for example, in a vector which includes appropriate control sequences. The host cell can be, for example, a higher eukaryotic cell, such as a mammalian cell, or a lower eukaryotic cell, such as a yeast cell, or the host cell can be a prokaryotic cell, such as a bacterial cell. Introduction of a construct (vector) into the host cell can be effected by, for example, calcium phosphate transfection, DEAE-Dextran mediated transfection, or electroporation.

Recombinant polypeptides can be expressed in plants, allowing for the production of crops of such plants and, therefore, the ability to conveniently produce large amounts of a desired product, such as a fatty acid or glycerol lipid. Accordingly, the methods of the disclosure can be practiced using any plant, including, for example, microalga and macroalgae, (such as marine algae and seaweeds), as well as plants that grow in soil.

In one embodiment, the host cell is a plant. The term “plant” is used broadly herein to refer to a eukaryotic organism containing plastids, such as chloroplasts, and includes any such organism at any stage of development, or to part of a plant, including a plant cutting, a plant cell, a plant cell culture, a plant organ, a plant seed, and a plantlet. A plant cell is the structural and physiological unit of the plant, comprising a protoplast and a cell wall. A plant cell can be in the form of an isolated single cell or a cultured cell, or can be part of higher organized unit, for example, a plant tissue, plant organ, or plant. Thus, a plant cell can be a protoplast, a gamete producing cell, or a cell or collection of cells that can regenerate into a whole plant. As such, a seed, which comprises multiple plant cells and is capable of regenerating into a whole plant, is considered plant cell for purposes of this disclosure. A plant tissue or plant organ can be a seed, protoplast, callus, or any other groups of plant cells that is organized into a structural or functional unit. Particularly useful parts of a plant include harvestable parts and parts useful for propagation of progeny plants. A harvestable part of a plant can be any useful part of a plant, for example, flowers, pollen, seedlings, tubers, leaves, stems, fruit, seeds, and roots. A part of a plant useful for propagation includes, for example, seeds, fruits, cuttings, seedlings, tubers, and rootstocks.

The genes of the present disclosure can be expressed in a higher plant. For example, Arabidopsis thaliana. The SN genes can also be expressed in a Brassica, Glycine, Gossypium, Medicago, Zea, Sorghum, Oryza, Triticum, or Panicum species.

A method of the disclosure can generate a plant containing genomic DNA (for example, a nuclear and/or plastid genomic DNA) that is genetically modified to contain a stably integrated polynucleotide (for example, as described in Hager and Bock, Appl. Microbiol. Biotechnol. 54:302-310, 2000). Accordingly, the present disclosure further provides a transgenic plant, e.g. C. reinhardtii, which comprises one or more chloroplasts containing a polynucleotide encoding one or more exogenous or endogenous polypeptides, including polypeptides that can allow for secretion of fuel products and/or fuel product precursors (e.g., isoprenoids, fatty acids, lipids, triglycerides). A photosynthetic organism of the present disclosure comprises at least one host cell that is modified to generate, for example, a fuel product or a fuel product precursor.

Some of the host organisms useful in the disclosed embodiments are, for example, are extremophiles, such as hyperthermophiles, psychrophiles, psychrotrophs, halophiles, barophiles and acidophiles. Some of the host organisms which may be used to practice the present disclosure are halophilic (e.g., Dunaliella salina, D. viridis, or D. tertiolecta). For example, D. salina can grow in ocean water and salt lakes (for example, salinity from 30-300 parts per thousand) and high salinity media (e.g., artificial seawater medium, seawater nutrient agar, brackish water medium, and seawater medium). In some embodiments of the disclosure, a host cell expressing a protein of the present disclosure can be grown in a liquid environment which is, for example, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3 molar or higher concentrations of sodium chloride. One of skill in the art will recognize that other salts (sodium salts, calcium salts, potassium salts, or other salts) may also be present in the liquid environments.

Where a halophilic organism is utilized for the present disclosure, it may be transformed with any of the vectors described herein. For example, D. salina may be transformed with a vector which is capable of insertion into the chloroplast or nuclear genome and which contains nucleic acids which encode a protein (e.g., an SN protein). Transformed halophilic organisms may then be grown in high-saline environments (e.g., salt lakes, salt ponds, and high-saline media) to produce the products (e.g., lipids) of interest. Isolation of the products may involve removing a transformed organism from a high-saline environment prior to extracting the product from the organism. In instances where the product is secreted into the surrounding environment, it may be necessary to desalinate the liquid environment prior to any further processing of the product.

The present disclosure further provides compositions comprising a genetically modified host cell. A composition comprises a genetically modified host cell; and will in some embodiments comprise one or more further components, which components are selected based in part on the intended use of the genetically modified host cell. Suitable components include, but are not limited to, salts; buffers; stabilizers; protease-inhibiting agents; cell membrane- and/or cell wall-preserving compounds, e.g., glycerol and dimethylsulfoxide; and nutritional media appropriate to the cell.

A host cell or host organism can be genetically modified, thus becoming a transgenic host cell or transgenic host organism. The plastid of a host cell or host organism can be genetically modified, thus becoming a transgenic plastid.

Culturing of Cells or Organisms

An organism may be grown under conditions which permit photosynthesis, however, this is not a requirement (e.g., a host organism may be grown in the absence of light). In some instances, the host organism may be genetically modified in such a way that its photosynthetic capability is diminished or destroyed. In growth conditions where a host organism is not capable of photosynthesis (e.g., because of the absence of light and/or genetic modification), typically, the organism will be provided with the necessary nutrients to support growth in the absence of photosynthesis. For example, a culture medium in (or on) which an organism is grown, may be supplemented with any required nutrient, including an organic carbon source, nitrogen source, phosphorous source, vitamins, metals, lipids, nucleic acids, micronutrients, and/or an organism-specific requirement. Organic carbon sources include any source of carbon which the host organism is able to metabolize including, but not limited to, acetate, simple carbohydrates (e.g., glucose, sucrose, and lactose), complex carbohydrates (e.g., starch and glycogen), proteins, and lipids. One of skill in the art will recognize that not all organisms will be able to sufficiently metabolize a particular nutrient and that nutrient mixtures may need to be modified from one organism to another in order to provide the appropriate nutrient mix.

Optimal growth of organisms occurs usually at a temperature of about 20° C. to about 25° C., although some organisms can still grow at a temperature of up to about 35° C. Active growth is typically performed in liquid culture. If the organisms are grown in a liquid medium and are shaken or mixed, the density of the cells can be anywhere from about 1 to 5×10⁸ cells/ml at the stationary phase. For example, the density of the cells at the stationary phase for

Chlamydomonas sp. can be about 1 to 5×10⁷ cells/ml; the density of the cells at the stationary phase for Nannochloropsis sp. can be about 1 to 5×10⁸ cells/ml; the density of the cells at the stationary phase for Scenedesmus sp. can be about 1 to 5×10⁷ cells/ml: and the density of the cells at the stationary phase for Chlorella sp. can be about 1 to 5×10⁸ cells/ml. Exemplary cell densities at the stationary phase are as follows: Chlamydomonas sp. can be about 1×10⁷ cells/ml: Nannochloropsis sp. can be about 1×10⁸ cells/ml; Scenedesmus sp. can be about 1×10⁷ cells/ml; and Chlorella sp. can be about 1×10⁸ cells/ml. An exemplary growth rate may yield, for example, a two to twenty fold increase in cells per day, depending on the growth conditions. In addition, doubling times for organisms can be, for example, 5 hours to 30 hours.

The organism can also be grown on solid media, for example, media containing about 1.5% agar, in plates or in slants.

One source of energy is fluorescent light that can be placed, for example, at a distance of about 1 inch to about two feet from the organism. Examples of types of fluorescent lights includes, for example, cool white and daylight. Bubbling with air or CO₂ improves the growth rate of the organism. Bubbling with CO₂ can be, for example, at 1% to 5% CO₂. If the lights are turned on and off at regular intervals (for example, 12:12 or 14:10 hours of light:dark) the cells of some organisms will become synchronized.

Long term storage of organisms can be achieved by streaking them onto plates, sealing the plates with, for example, Parafilm™, and placing them in dim light at about 10° C. to about 18° C. Alternatively, organisms may be grown as streaks or stabs into agar tubes, capped, and stored at about 10° C. to about 18° C. Both methods allow for the storage of the organisms for several months.

For longer storage, the organisms can be grown in liquid culture to mid to late log phase and then supplemented with a penetrating cryoprotective agent like DMSO or MeOH, and stored at less than −130° C. An exemplary range of DMSO concentrations that can be used is 5 to 8%. An exemplary range of MeOH concentrations that can be used is 3 to 9%.

Organisms can be grown on a defined minimal medium (for example, high salt medium (HSM), modified artificial sea water medium (MASM), or F/2 medium) with light as the sole energy source. In other instances, the organism can be grown in a medium (for example, tris acetate phosphate (TAP) medium), and supplemented with an organic carbon source.

Organisms, such as algae, can grow naturally in fresh water or marine water. Culture media for freshwater algae can be, for example, synthetic media, enriched media, soil water media, and solidified media, such as agar. Various culture media have been developed and used for the isolation and cultivation of fresh water algae and are described in Watanabe, M. W. (2005). Freshwater Culture Media. In R. A. Andersen (Ed.), Algal Culturing Techniques (pp. 13-20). Elsevier Academic Press. Culture media for marine algae can be, for example, artificial seawater media or natural seawater media. Guidelines for the preparation of media are described in Harrison, P. J. and Berges, J. A. (2005). Marine Culture Media. In R. A. Andersen (Ed.), Algal Culturing Techniques (pp. 21-33). Elsevier Academic Press.

Organisms may be grown in outdoor open water, such as ponds, the ocean, seas, rivers, waterbeds, marshes, shallow pools, lakes, aqueducts, and reservoirs. When grown in water, the organism can be contained in a halo-like object comprised of lego-like particles. The halo-like object encircles the organism and allows it to retain nutrients from the water beneath while keeping it in open sunlight.

In some instances, organisms can be grown in containers wherein each container comprises one or two organisms, or a plurality of organisms. The containers can be configured to float on water. For example, a container can be filled by a combination of air and water to make the container and the organism(s) in it buoyant. An organism that is adapted to grow in fresh water can thus be grown in salt water (i.e., the ocean) and vice versa. This mechanism allows for automatic death of the organism if there is any damage to the container.

Culturing techniques for algae are well know to one of skill in the art and are described, for example, in Freshwater Culture Media. In R. A. Andersen (Ed.), Algal Culturing Techniques. Elsevier Academic Press.

Because photosynthetic organisms, for example, algae, require sunlight, CO₂ and water for growth, they can be cultivated in, for example, open ponds and lakes. However, these open systems are more vulnerable to contamination than a closed system. One challenge with using an open system is that the organism of interest may not grow as quickly as a potential invader. This becomes a problem when another organism invades the liquid environment in which the organism of interest is growing, and the invading organism has a faster growth rate and takes over the system.

In addition, in open systems there is less control over water temperature. CO₂ concentration, and lighting conditions. The growing season of the organism is largely dependent on location and, aside from tropical areas, is limited to the warmer months of the year. In addition, in an open system, the number of different organisms that can be grown is limited to those that are able to survive in the chosen location. An open system, however, is cheaper to set up and/or maintain than a closed system.

Another approach to growing an organism is to use a semi-closed system, such as covering the pond or pool with a structure, for example, a “greenhouse-type” structure. While this can result in a smaller system, it addresses many of the problems associated with an open system. The advantages of a semi-closed system are that it can allow for a greater number of different organisms to be grown, it can allow for an organism to be dominant over an invading organism by allowing the organism of interest to out compete the invading organism for nutrients required for its growth, and it can extend the growing season for the organism. For example, if the system is heated, the organism can grow year round.

A variation of the pond system is an artificial pond, for example, a raceway pond. In these ponds, the organism, water, and nutrients circulate around a “racetrack.” Paddlewheels provide constant motion to the liquid in the racetrack, allowing for the organism to be circulated back to the surface of the liquid at a chosen frequency. Paddlewheels also provide a source of agitation and oxygenate the system. These raceway ponds can be enclosed, for example, in a building or a greenhouse, or can be located outdoors.

Raceway ponds are usually kept shallow because the organism needs to be exposed to sunlight, and sunlight can only penetrate the pond water to a limited depth. The depth of a raceway pond can be, for example, about 4 to about 12 inches. In addition, the volume of liquid that can be contained in a raceway pond can be, for example, about 200 liters to about 600,000 liters.

The raceway ponds can be operated in a continuous manner, with, for example, CO₂ and nutrients being constantly fed to the ponds, while water containing the organism is removed at the other end.

If the raceway pond is placed outdoors, there are several different ways to address the invasion of an unwanted organism. For example, the pH or salinity of the liquid in which the desired organism is in can be such that the invading organism either slows down its growth or dies.

Also, chemicals can be added to the liquid, such as bleach, or a pesticide can be added to the liquid, such as glyphosate. In addition, the organism of interest can be genetically modified such that it is better suited to survive in the liquid environment. Any one or more of the above strategies can be used to address the invasion of an unwanted organism.

Alternatively, organisms, such as algae, can be grown in closed structures such as photobioreactors, where the environment is under stricter control than in open systems or semi-closed systems. A photobioreactor is a bioreactor which incorporates some type of light source to provide photonic energy input into the reactor. The term photobioreactor can refer to a system closed to the environment and having no direct exchange of gases and contaminants with the environment. A photobioreactor can be described as an enclosed, illuminated culture vessel designed for controlled biomass production of phototrophic liquid cell suspension cultures. Examples of photobioreactors include, for example, glass containers, plastic tubes, tanks, plastic sleeves, and bags. Examples of light sources that can be used to provide the energy required to sustain photosynthesis include, for example, fluorescent bulbs, LEDs, and natural sunlight. Because these systems are closed everything that the organism needs to grow (for example, carbon dioxide, nutrients, water, and light) must be introduced into the bioreactor.

Photobioreactors, despite the costs to set up and maintain them, have several advantages over open systems, they can, for example, prevent or minimize contamination, permit axenic organism cultivation of monocultures (a culture consisting of only one species of organism), offer better control over the culture conditions (for example, pH, light, carbon dioxide, and temperature), prevent water evaporation, lower carbon dioxide losses due to out gassing, and permit higher cell concentrations.

On the other hand, certain requirements of photobioreactors, such as cooling, mixing, control of oxygen accumulation and biofouling, make these systems more expensive to build and operate than open systems or semi-closed systems.

Photobioreactors can be set up to be continually harvested (as is with the majority of the larger volume cultivation systems), or harvested one batch at a time (for example, as with polyethlyene bag cultivation). A batch photobioreactor is set up with, for example, nutrients, an organism (for example, algae), and water, and the organism is allowed to grow until the batch is harvested. A continuous photobioreactor can be harvested, for example, either continually, daily, or at fixed time intervals.

High density photobioreactors are described in, for example, Lee, et al., Biotech. Bioengineering 44:1161-1167, 1994. Other types of bioreactors, such as those for sewage and waste water treatments, are described in, Sawayama. et al., Appl. Micro. Biotech., 41:729-731, 1994. Additional examples of photobioreactors are described in, U.S. Appl. Publ. No. 2005/0260553, U.S. Pat. No. 5,958,761, and U.S. Pat. No. 6,083,740. Also, organisms, such as algae may be mass-cultured for the removal of heavy metals (for example, as described in Wilkinson, Biotech. Letters, 11:861-864, 1989), hydrogen (for example, as described in U.S. Patent Application Publication No. 2003/0162273), and pharmaceutical compounds from a water, soil, or other source or sample. Organisms can also be cultured in conventional fermentation bioreactors, which include, but are not limited to, batch, fed-batch, cell recycle, and continuous fermentors. Additional methods of culturing organisms and variations of the methods described herein are known to one of skill in the art.

Organisms can also be grown near ethanol production plants or other facilities or regions (e.g., cities and highways) generating CO₂. As such, the methods herein contemplate business methods for selling carbon credits to ethanol plants or other facilities or regions generating CO₂ while making fuels or fuel products by growing one or more of the organisms described herein near the ethanol production plant, facility, or region.

The organism of interest, grown in any of the systems described herein, can be, for example, continually harvested, or harvested one batch at a time.

CO₂ can be delivered to any of the systems described herein, for example, by bubbling in CO₂ from under the surface of the liquid containing the organism. Also, sparges can be used to inject CO₂ into the liquid. Spargers are, for example, porous disc or tube assemblies that are also referred to as Bubblers, Carbonators, Aerators, Porous Stones and Diffusers.

Nutrients that can be used in the systems described herein include, for example, nitrogen (in the form of NO₃⁻ or NH₄⁺), phosphorus, and trace metals (Fe, Mg, K, Ca, Co, Cu, Mn, Mo, Zn, V, and B). The nutrients can come, for example, in a solid form or in a liquid form. If the nutrients are in a solid form they can be mixed with, for example, fresh or salt water prior to being delivered to the liquid containing the organism, or prior to being delivered to a photobioreactor.

Organisms can be grown in cultures, for example large scale cultures, where large scale cultures refers to growth of cultures in volumes of greater than about 6 liters, or greater than about 10 liters, or greater than about 20 liters. Large scale growth can also be growth of cultures in volumes of 50 liters or more, 100 liters or more, or 200 liters or more. Large scale growth can be growth of cultures in, for example, ponds, containers, vessels, or other areas, where the pond, container, vessel, or area that contains the culture is for example, at lease 5 square meters, at least 10 square meters, at least 200 square meters, at least 500 square meters, at least 1,500 square meters, at least 2,500 square meters, in area, or greater.

Chlamydomonas sp., Nannochloropsis sp., Scenedesmus sp., and Chlorella sp. are exemplary algae that can be cultured as described herein and can grow under a wide array of conditions. One organism that can be cultured as described herein is a commonly used laboratory species C. reinhardtii. Cells of this species are haploid, and can grow on a simple medium of inorganic salts, using photosynthesis to provide energy. This organism can also grow in total darkness if acetate is provided as a carbon source. C. reinhardtii can be readily grown at room temperature under standard fluorescent lights. In addition, the cells can be synchronized by placing them on a light-dark cycle. Other methods of culturing C. reinhardtii cells are known to one of skill in the art.

Polynucleotides and Polypeptides

Also provided are isolated polynucleotides encoding a protein, for example, an SN protein described herein. As used herein “isolated polynucleotide” means a polynucleotide that is free of one or both of the nucleotide sequences which flank the polynucleotide in the naturally-occurring genome of the organism from which the polynucleotide is derived. The term includes, for example, a polynucleotide or fragment thereof that is incorporated into a vector or expression cassette; into an autonomously replicating plasmid or virus; into the genomic DNA of a prokaryote or eukaryote; or that exists as a separate molecule independent of other polynucleotides. It also includes a recombinant polynucleotide that is part of a hybrid polynucleotide, for example, one encoding a polypeptide sequence.

The novel proteins of the present disclosure can be made by any method known in the art. The protein may be synthesized using either solid-phase peptide synthesis or by classical solution peptide synthesis also known as liquid-phase peptide synthesis. Using Val-Pro-Pro, Enalapril and Lisinopril as starting templates, several series of peptide analogs such as X-Pro-Pro. X-Ala-Pro, and X-Lys-Pro, wherein X represents any amino acid residue, may be synthesized using solid-phase or liquid-phase peptide synthesis. Methods for carrying out liquid phase synthesis of libraries of peptides and oligonucleotides coupled to a soluble oligomeric support have also been described. Bayer, Ernst and Mutter, Manfred, Nature 237:512-513 (1972); Bayer, Ernst, et al., J. Am. Chem. Soc. 96:7333-7336 (1974); Bonora, Gian Maria, et al., Nucleic Acids Res. 18:3155-3159 (1990). Liquid phase synthetic methods have the advantage over solid phase synthetic methods in that liquid phase synthesis methods do not require a structure present on a first reactant which is suitable for attaching the reactant to the solid phase. Also, liquid phase synthesis methods do not require avoiding chemical conditions which may cleave the bond between the solid phase and the first reactant (or intermediate product). In addition, reactions in a homogeneous solution may give better yields and more complete reactions than those obtained in heterogeneous solid phase/liquid phase systems such as those present in solid phase synthesis.

In oligomer-supported liquid phase synthesis the growing product is attached to a large soluble polymeric group. The product from each step of the synthesis can then be separated from unreacted reactants based on the large difference in size between the relatively large polymer-attached product and the unreacted reactants. This permits reactions to take place in homogeneous solutions, and eliminates tedious purification steps associated with traditional liquid phase synthesis. Oligomer-supported liquid phase synthesis has also been adapted to automatic liquid phase synthesis of peptides. Bayer, Ernst, et al., Peptides: Chemistry, Structure, Biology, 426-432.

For solid-phase peptide synthesis, the procedure entails the sequential assembly of the appropriate amino acids into a peptide of a desired sequence while the end of the growing peptide is linked to an insoluble support. Usually, the carboxyl terminus of the peptide is linked to a polymer from which it can be liberated upon treatment with a cleavage reagent. In a common method, an amino acid is bound to a resin particle, and the peptide generated in a stepwise manner by successive additions of protected amino acids to produce a chain of amino acids. Modifications of the technique described by Merrifield are commonly used. See, e.g., Merrifield, J. Am. Chem. Soc. 96: 2989-93 (1964). In an automated solid-phase method, peptides are synthesized by loading the carboxy-terminal amino acid onto an organic linker (e.g., PAM, 4-oxymethylphenylacetamidomethyl), which is covalently attached to an insoluble polystyrene resin cross-linked with divinyl benzene. The terminal amine may be protected by blocking with t-butyloxycarbonyl. Hydroxyl- and carboxyl-groups are commonly protected by blocking with O-benzyl groups. Synthesis is accomplished in an automated peptide synthesizer, such as that available from Applied Biosystems (Foster City, Calif.). Following synthesis, the product may be removed from the resin. The blocking groups are removed by using hydrofluoric acid or trifluoromethyl sulfonic acid according to established methods. A routine synthesis may produce 0.5 mmole of peptide resin. Following cleavage and purification, a yield of approximately 60 to 70% is typically produced. Purification of the product peptides is accomplished by, for example, crystallizing the peptide from an organic solvent such as methyl-butyl ether, then dissolving in distilled water, and using dialysis (if the molecular weight of the subject peptide is greater than about 500 daltons) or reverse high pressure liquid chromatography (e.g., using a C¹⁸ column with 0.1% trifluoroacetic acid and acetonitrile as solvents) if the molecular weight of the peptide is less than 500 daltons. Purified peptide may be lyophilized and stored in a dry state until use. Analysis of the resulting peptides may be accomplished using the common methods of analytical high pressure liquid chromatography (HPLC) and electrospray mass spectrometry (ES-MS).

In other cases, a protein, for example, an SN protein, is produced by recombinant methods. For production of any of the proteins described herein, host cells transformed with an expression vector containing the polynucleotide encoding such a protein can be used. The host cell can be a higher eukaryotic cell, such as a mammalian cell, or a lower eukaryotic cell such as a yeast or algal cell, or the host can be a prokaryotic cell such as a bacterial cell. Introduction of the expression vector into the host cell can be accomplished by a variety of methods including calcium phosphate transfection. DEAE-dextran mediated transfection, polybrene, protoplast fusion, liposomes, direct microinjection into the nuclei, scrape loading, biolistic transformation and electroporation. Large scale production of proteins from recombinant organisms is a well established process practiced on a commercial scale and well within the capabilities of one skilled in the art.

The polynucleotide sequence can comprise at least one mutation comprising one or more nucleotide additions, deletions or substitutions. The at least one mutation can be in a coding region, can result in one or more amino acid additions, deletions or substitutions in a protein encoded by the coding region, can be in a regulatory region, can be in a 5′ UTR, can be in a 3′ UTR, and/or can be in a promoter.

It should be recognized that the present disclosure is not limited to transgenic cells, organisms, and plastids containing a protein or proteins as disclosed herein, but also encompasses such cells, organisms, and plastids transformed with additional nucleotide sequences encoding enzymes involved in fatty acid synthesis. Thus, some embodiments involve the introduction of one or more sequences encoding proteins involved in fatty acid synthesis in addition to a protein disclosed herein. For example, several enzymes in a fatty acid production pathway may be linked, either directly or indirectly, such that products produced by one enzyme in the pathway, once produced, are in close proximity to the next enzyme in the pathway. These additional sequences may be contained in a single vector either operatively linked to a single promoter or linked to multiple promoters, e.g. one promoter for each sequence. Alternatively, the additional coding sequences may be contained in a plurality of additional vectors. When a plurality of vectors are used, they can be introduced into the host cell or organism simultaneously or sequentially.

Additional embodiments provide a plastid, and in particular a chloroplast, transformed with a polynucleotide encoding a protein of the present disclosure. The protein may be introduced into the genome of the plastid using any of the methods described herein or otherwise known in the art. The plastid may be contained in the organism in which it naturally occurs. Alternatively, the plastid may be an isolated plastid, that is, a plastid that has been removed from the cell in which it normally occurs. Methods for the isolation of plastids are known in the art and can be found, for example, in Maliga et al., Methods in Plant Molecular Biology, Cold Spring Harbor Laboratory Press. 1995: Gupta and Singh, J. Biosci., 21:819 (1996); and Camara et al., Plant Physiol., 73:94 (1983). The isolated plastid transformed with a protein of the present disclosure can be introduced into a host cell. The host cell can be one that naturally contains the plastid or one in which the plastid is not naturally found.

Also within the scope of the present disclosure are artificial plastid genomes, for example chloroplast genomes, that contain nucleotide sequences encoding any one or more of the proteins of the present disclosure. Methods for the assembly of artificial plastid genomes can be found in co-pending U.S. patent application Ser. No. 12/287,230 filed Oct. 6, 2008, published as U.S. Publication No. 2009/0123977 on May 14, 2009, and U.S. patent application Ser. No. 12/384,893 filed Apr. 8, 2009, published as U.S. Publication No. 2009/0269816 on Oct. 29, 2009, each of which is incorporated by reference in its entirety.

One or more nucleotides of the present disclosure can also be modified such that the resulting amino acid is “substantially identical” to the unmodified or reference amino acid.

A “substantially identical” amino acid sequence is a sequence that differs from a reference sequence by one or more conservative or non-conservative amino acid substitutions, deletions, or insertions, particularly when such a substitution occurs at a site that is not the active site (catalytic domains (CDs)) of the molecule and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine).

The disclosure provides alternative embodiments of the polypeptides of the invention (and the nucleic acids that encode them) comprising at least one conservative amino acid substitution, as discussed herein (e.g., conservative amino acid substitutions are those that substitute a given amino acid in a polypeptide by another amino acid of like characteristics). The invention provides polypeptides (and the nucleic acids that encode them) wherein any, some or all amino acids residues are substituted by another amino acid of like characteristics, e.g., a conservative amino acid substitution.

Conservative substitutions are those that substitute a given amino acid in a polypeptide by another amino acid of like characteristics. Examples of conservative substitutions are the following replacements: replacements of an aliphatic amino acid such as Alanine, Valine, Leucine and Isoleucine with another aliphatic amino acid; replacement of a Serine with a Threonine or vice versa; replacement of an acidic residue such as Aspartic acid and Glutamic acid with another acidic residue; replacement of a residue bearing an amide group, such as Asparagine and Glutamine, with another residue bearing an amide group; exchange of a basic residue such as Lysine and Arginine with another basic residue; and replacement of an aromatic residue such as Phenylalanine, Tyrosine with another aromatic residue. In alternative aspects, these conservative substitutions can also be synthetic equivalents of these amino acids.

Introduction of Polynucleotide into a Host Organism or Cell

To generate a genetically modified host cell, a polynucleotide, or a polynucleotide cloned into a vector, is introduced stably or transiently into a host cell, using established techniques, including, but not limited to, electroporation, calcium phosphate precipitation, DEAE-dextran mediated transfection, and liposome-mediated transfection. For transformation, a polynucleotide of the present disclosure will generally further include a selectable marker, e.g., any of several well-known selectable markers such as neomycin resistance, ampicillin resistance, tetracycline resistance, chloramphenicol resistance, and kanamycin resistance.

A polynucleotide or recombinant nucleic acid molecule described herein, can be introduced into a cell (e.g., alga cell) using any method known in the art. A polynucleotide can be introduced into a cell by a variety of methods, which are well known in the art and selected, in part, based on the particular host cell. For example, the polynucleotide can be introduced into a cell using a direct gene transfer method such as electroporation or microprojectile mediated (biolistic) transformation using a particle gun, or the “glass bead method,” or by pollen-mediated transformation, liposome-mediated transformation, transformation using wounded or enzyme-degraded immature embryos, or wounded or enzyme-degraded embryogenic callus (for example, as described in Potrykus, Ann. Rev. Plant. Physiol. Plant Mol. Biol. 42:205-225, 1991).

As discussed above, microprojectile mediated transformation can be used to introduce a polynucleotide into a cell (for example, as described in Klein et al., Nature 327:70-73, 1987).

This method utilizes microprojectiles such as gold or tungsten, which are coated with the desired polynucleotide by precipitation with calcium chloride, spermidine or polyethylene glycol. The microprojectile particles are accelerated at high speed into a cell using a device such as the BIOLISTIC PD-1000 particle gun (BioRad; Hercules Calif.). Methods for the transformation using biolistic methods are well known in the art (for example, as described in Christou, Trends in Plant Science 1:423-431, 1996). Microprojectile mediated transformation has been used, for example, to generate a variety of transgenic plant species, including cotton, tobacco, corn, hybrid poplar and papaya. Important cereal crops such as wheat, oat, barley, sorghum and rice also have been transformed using microprojectile mediated delivery (for example, as described in Duan et al., Nature Biotech. 14:494-498, 1996; and Shimamoto. Curr. Opin. Biotech. 5:158-162, 1994). The transformation of most dicotyledonous plants is possible with the methods described above. Transformation of monocotyledonous plants also can be transformed using, for example, biolistic methods as described above, protoplast transformation, electroporation of partially permeabilized cells, introduction of DNA using glass fibers, and the glass bead agitation method.

The basic techniques used for transformation and expression in photosynthetic microorganisms are similar to those commonly used for E. coli, Saccharomyces cerevisiae and other species. Transformation methods customized for a photosynthetic microorganisms, e.g., the chloroplast of a strain of algae, are known in the art. These methods have been described in a number of texts for standard molecular biological manipulation (see Packer & Glaser, 1988, “Cyanobacteria”, Meth. Enzymol., Vol. 167; Weissbach & Weissbach. 1988. “Methods for plant molecular biology,” Academic Press, New York, Sambrook, Fritsch & Maniatis, 1989, “Molecular Cloning: A laboratory manual,” 2nd edition Cold Spring Harbor Laboratory Press, Cold Spring Harbor, N.Y.; and Clark M S, 1997, Plant Molecular Biology, Springer, N.Y.). These methods include, for example, biolistic devices (See, for example, Sanford, Trends In Biotech. (1988) 6: 299-302, U.S. Pat. No. 4,945,050; electroporation (Fromm et al., Proc. Nat'l. Acad. Sci. (USA) (1985) 82: 5824-5828); use of a laser beam, electroporation, microinjection or any other method capable of introducing DNA into a host cell.

Plastid transformation is a routine and well known method for introducing a polynucleotide into a plant cell chloroplast (see U.S. Pat. Nos. 5,451,513, 5,545,817, and 5,545,818; WO 95/16783; McBride et al., Proc. Natl. Acad Sci., USA 91:7301-7305, 1994). In some embodiments, chloroplast transformation involves introducing regions of chloroplast DNA flanking a desired nucleotide sequence, allowing for homologous recombination of the exogenous DNA into the target chloroplast genome. In some instances one to 1.5 kb flanking nucleotide sequences of chloroplast genomic DNA may be used. Using this method, point mutations in the chloroplast 16S rRNA and rps12 genes, which confer resistance to spectinomycin and streptomycin, can be utilized as selectable markers for transformation (Svab et al., Proc. Natl. Acad. Sci., USA 87:8526-8530, 1990), and can result in stable homoplasmic transformants, at a frequency of approximately one per 100 bombardments of target leaves.

A further refinement in chloroplast transformation/expression technology that facilitates control over the timing and tissue pattern of expression of introduced DNA coding sequences in plant plastid genomes has been described in PCT International Publication WO 95/16783 and U.S. Pat. No. 5,576,198. This method involves the introduction into plant cells of constructs for nuclear transformation that provide for the expression of a viral single subunit RNA polymerase and targeting of this polymerase into the plastids via fusion to a plastid transit peptide. Transformation of plastids with DNA constructs comprising a viral single subunit RNA polymerase-specific promoter specific to the RNA polymerase expressed from the nuclear expression constructs operably linked to DNA coding sequences of interest permits control of the plastid expression constructs in a tissue and/or developmental specific manner in plants comprising both the nuclear polymerase construct and the plastid expression constructs.

Expression of the nuclear RNA polymerase coding sequence can be placed under the control of either a constitutive promoter, or a tissue- or developmental stage-specific promoter, thereby extending this control to the plastid expression construct responsive to the plastid-targeted, nuclear-encoded viral RNA polymerase.

When nuclear transformation is utilized, the protein can be modified for plastid targeting by employing plant cell nuclear transformation constructs wherein DNA coding sequences of interest are fused to any of the available transit peptide sequences capable of facilitating transport of the encoded enzymes into plant plastids, and driving expression by employing an appropriate promoter. Targeting of the protein can be achieved by fusing DNA encoding plastid, e.g., chloroplast, leucoplast, amyloplast, etc., transit peptide sequences to the 5′ end of DNAs encoding the enzymes. The sequences that encode a transit peptide region can be obtained, for example, from plant nuclear-encoded plastid proteins, such as the small subunit (SSU) of ribulose bisphosphate carboxylase, EPSP synthase, plant fatty acid biosynthesis related genes including fatty acyl-ACP thioesterases, acyl carrier protein (ACP), stearoyl-ACP desaturase, β-ketoacyl-ACP synthase and acyl-ACP thioesterase, or LHCPII genes, etc. Plastid transit peptide sequences can also be obtained from nucleic acid sequences encoding carotenoid biosynthetic enzymes, such as GGPP synthase, phytoene synthase, and phytoene desaturase. Other transit peptide sequences are disclosed in Von Heijne et al. (1991) Plant Mol. Biol. Rep. 9: 104; Clark et al. (1989) J. Biol. Chem. 264: 17544; della-Cioppa et al. (1987) Plant Physiol. 84: 965; Romer et al. (1993) Biochem. Biophys. Res. Commun. 196: 1414; and Shah et al. (1986) Science 233: 478. Another transit peptide sequence is that of the intact ACCase from Chlamydomonas (genbank EDO96563, amino acids 1-33). The encoding sequence for a transit peptide effective in transport to plastids can include all or a portion of the encoding sequence for a particular transit peptide, and may also contain portions of the mature protein encoding sequence associated with a particular transit peptide. Numerous examples of transit peptides that can be used to deliver target proteins into plastids exist, and the particular transit peptide encoding sequences useful in the present disclosure are not critical as long as delivery into a plastid is obtained. Proteolytic processing within the plastid then produces the mature enzyme. This technique has proven successful with enzymes involved in polyhydroxyalkanoate biosynthesis (Nawrath et al. (1994) Proc. Natl. Acad Sci. USA 91: 12760), and neomycin phosphotransferase II (NPT-II) and CP4 EPSPS (Padgette et al. (1995) Crop Sci. 35: 1451), for example.

Of interest are transit peptide sequences derived from enzymes known to be imported into the leucoplasts of seeds. Examples of enzymes containing useful transit peptides include those related to lipid biosynthesis (e.g., subunits of the plastid-targeted dicot acetyl-CoA carboxylase, biotin carboxylase, biotin carboxyl carrier protein, α-carboxy-transferase, and plastid-targeted monocot multifunctional acetyl-CoA carboxylase (Mw, 220,000); plastidic subunits of the fatty acid synthase complex (e.g., acyl carrier protein (ACP), malonyl-ACP synthase, KASI, KASII, and KASIII); steroyl-ACP desaturase; thioesterases (specific for short, medium, and long chain acyl ACP); plastid-targeted acyl transferases (e.g., glycerol-3-phosphate and acyl transferase): enzymes involved in the biosynthesis of aspartate family amino acids; phytoene synthase; gibberellic acid biosynthesis (e.g., ent-kaurene synthases 1 and 2); and carotenoid biosynthesis (e.g., lycopene synthase).

In some embodiments, an alga is transformed with a nucleic acid which encodes a protein of interest, for example, an SN protein.

In one embodiment, a transformation may introduce a nucleic acid into a plastid of the host alga (e.g., chloroplast). In another embodiment, a transformation may introduce a nucleic acid into the nuclear genome of the host alga. In still another embodiment, a transformation may introduce nucleic acids into both the nuclear genome and into a plastid.

Transformed cells can be plated on selective media following introduction of exogenous nucleic acids. This method may also comprise several steps for screening. A screen of primary transformants can be conducted to determine which clones have proper insertion of the exogenous nucleic acids. Clones which show the proper integration may be propagated and re-screened to ensure genetic stability. Such methodology ensures that the transformants contain the genes of interest. In many instances, such screening is performed by polymerase chain reaction (PCR); however, any other appropriate technique known in the art may be utilized. Many different methods of PCR are known in the art (e.g., nested PCR, real time PCR). For any given screen, one of skill in the art will recognize that PCR components may be varied to achieve optimal screening results. For example, magnesium concentration may need to be adjusted upwards when PCR is performed on disrupted alga cells to which (which chelates magnesium) is added to chelate toxic metals. Following the screening for clones with the proper integration of exogenous nucleic acids, clones can be screened for the presence of the encoded protein(s) and/or products. Protein expression screening can be performed by Western blot analysis and/or enzyme activity assays. Transporter and/or product screening may be performed by any method known in the art, for example ATP turnover assay, substrate transport assay, HPLC or gas chromatography.

The expression of the protein or enzyme can be accomplished by inserting a polynucleotide sequence (gene) encoding the protein or enzyme into the chloroplast or nuclear genome of a microalgae. The modified strain of microalgae can be made homoplasmic to ensure that the polynucleotide will be stably maintained in the chloroplast genome of all descendents. A microalga is homoplasmic for a gene when the inserted gene is present in all copies of the chloroplast genome, for example. It is apparent to one of skill in the art that a chloroplast may contain multiple copies of its genome, and therefore, the term “homoplasmic” or “homoplasmy” refers to the state where all copies of a particular locus of interest are substantially identical. Plastid expression, in which genes are inserted by homologous recombination into all of the several thousand copies of the circular plastid genome present in each plant cell, takes advantage of the enormous copy number advantage over nuclear-expressed genes to permit expression levels that can readily exceed 10% or more of the total soluble plant protein. The process of determining the plasmic state of an organism of the present disclosure involves screening transformants for the presence of exogenous nucleic acids and the absence of wild-type nucleic acids at a given locus of interest.

Vectors

Construct, vector and plasmid are used interchangeably throughout the disclosure. Nucleic acids encoding the proteins described herein, can be contained in vectors, including cloning and expression vectors. A cloning vector is a self-replicating DNA molecule that serves to transfer a DNA segment into a host cell. Three common types of cloning vectors are bacterial plasmids, phages, and other viruses. An expression vector is a cloning vector designed so that a coding sequence inserted at a particular site will be transcribed and translated into a protein. Both cloning and expression vectors can contain nucleotide sequences that allow the vectors to replicate in one or more suitable host cells. In cloning vectors, this sequence is generally one that enables the vector to replicate independently of the host cell chromosomes, and also includes either origins of replication or autonomously replicating sequences.

In some embodiments, a polynucleotide of the present disclosure is cloned or inserted into an expression vector using cloning techniques know to one of skill in the art. The nucleotide sequences may be inserted into a vector by a variety of methods. In the most common method the sequences are inserted into an appropriate restriction endonuclease site(s) using procedures commonly known to those skilled in the art and detailed in, for example, Sambrook et al., Molecular Cloning. A Laboratory Manual. 2nd Ed., Cold Spring Harbor Press, (1989) and Ausubel et al., Short Protocols in Molecular Biology, 2nd Ed., John Wiley & Sons (1992).

Suitable expression vectors include, but are not limited to, baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, and herpes simplex virus), PI-based artificial chromosomes, yeast plasmids, yeast artificial chromosomes, and any other vectors specific for specific hosts of interest (such as E. coli and yeast). Thus, for example, a polynucleotide encoding an SN protein, can be inserted into any one of a variety of expression vectors that are capable of expressing the protein. Such vectors can include, for example, chromosomal, nonchromosomal and synthetic DNA sequences.

Suitable expression vectors include chromosomal, non-chromosomal and synthetic DNA sequences, for example, SV 40 derivatives; bacterial plasmids: phage DNA; baculovirus; yeast plasmids; vectors derived from combinations of plasmids and phage DNA; and viral DNA such as vaccinia, adenovirus, fowl pox virus, and pseudorabies. In addition, any other vector that is replicable and viable in the host may be used. For example, vectors such as Ble2A, Arg7/2A, and SEnuc357 can be used for the expression of a protein.

Numerous suitable expression vectors are known to those of skill in the art. The following vectors are provided by way of example; for bacterial host cells: pQE vectors (Qiagen), pBluescript plasmids, pNH vectors, lambda-ZAP vectors (Stratagene), pTrc99a, pKK223-3, pDR540, and pRIT2T (Pharmacia); for eukaryotic host cells: pXT1, pSGS (Stratagene), pSVK3, pBPV, pMSG, pET21a-d(+) vectors (Novagen), and pSVLSV40 (Pharmacia). However, any other plasmid or other vector may be used so long as it is compatible with the host cell.

The expression vector, or a linearized portion thereof, can encode one or more exogenous or endogenous nucleotide sequences. Examples of exogenous nucleotide sequences that can be transformed into a host include genes from bacteria, fungi, plants, photosynthetic bacteria or other algae. Examples of other types of nucleotide sequences that can be transformed into a host, include, but are not limited to, SN genes, transporter genes, isoprenoid producing genes, genes which encode for proteins which produce isoprenoids with two phosphates (e.g., GPP synthase and/or FPP synthase), genes which encode for proteins which produce fatty acids, lipids, or triglycerides, for example, ACCases, endogenous promoters, and 5′ UTRs from the psbA, atpA, or rbcL genes. In some instances, an exogenous sequence is flanked by two homologous sequences.

Homologous sequences are, for example, those that have at least 50%, at least 60%, at least 70%, at least 80%, at least 90%, at least 95%, at least 98%, or at least 99% sequence identity to a reference amino acid sequence or nucleotide sequence, for example, the amino acid sequence or nucleotide sequence that is found in the host cell from which the protein is naturally obtained from or derived from.

A nucleotide sequence can also be homologous to a codon-optimized gene sequence. For example, a nucleotide sequence can have, for example, at least 50%, at least 60%, at least 70%, at least 80%, at least 90%, at least 95%, at least 98%, or at least 99% nucleic acid sequence identity to the codon-optimized gene sequence.

The first and second homologous sequences enable recombination of the exogenous or endogenous sequence into the genome of the host organism. The first and second homologous sequences can be at least 100, at least 200, at least 300, at least 400, at least 500, or at least 1500 nucleotides in length.

In some embodiments, about 0.5 to about 1.5 kb flanking nucleotide sequences of chloroplast genomic DNA may be used. In other embodiments about 0.5 to about 1.5 kb flanking nucleotide sequences of nuclear genomic DNA may be used, or about 2.0 to about 5.0 kb may be used.

In some embodiments, the vector may comprise nucleotide sequences that are codon-biased for expression in the organism being transformed. In another embodiment, a gene of interest, for example, an SN gene, may comprise nucleotide sequences that are codon-biased for expression in the organism being transformed. In addition, the nucleotide sequence of a tag may be codon-biased or codon-optimized for expression in the organism being transformed.

A polynucleotide sequence may comprise nucleotide sequences that are codon biased for expression in the organism being transformed. The skilled artisan is well aware of the “codon-bias” exhibited by a specific host cell in usage of nucleotide codons to specify a given amino acid. Without being bound by theory, by using a host cell's preferred codons, the rate of translation may be greater. Therefore, when synthesizing a gene for improved expression in a host cell, it may be desirable to design the gene such that its frequency of codon usage approaches the frequency of preferred codon usage of the host cell. In some organisms, codon bias differs between the nuclear genome and organelle genomes, thus, codon optimization or biasing may be performed for the target genome (e.g., nuclear codon biased or chloroplast codon biased). In some embodiments, codon biasing occurs before mutagenesis to generate a polypeptide. In other embodiments, codon biasing occurs after mutagenesis to generate a polynucleotide. In yet other embodiments, codon biasing occurs before mutagenesis as well as after mutagenesis. Codon bias is described in detail herein.

In some embodiments, a vector comprises a polynucleotide operably linked to one or more control elements, such as a promoter and/or a transcription terminator. A nucleic acid sequence is operably linked when it is placed into a functional relationship with another nucleic acid sequence. For example, DNA for a presequence or secretory leader is operatively linked to DNA for a polypeptide if it is expressed as a preprotein which participates in the secretion of the polypeptide; a promoter is operably linked to a coding sequence if it affects the transcription of the sequence; or a ribosome binding site is operably linked to a coding sequence if it is positioned so as to facilitate translation. Generally, operably linked sequences are contiguous and, in the case of a secretory leader, contiguous and in reading phase. Linking is achieved by ligation at restriction enzyme sites. If suitable restriction sites are not available, then synthetic oligonucleotide adapters or linkers can be used as is known to those skilled in the art. Sambrook et al., Molecular Cloning, A Laboratory Manual, 2^{nd} Ed., Cold Spring Harbor Press, (1989) and Ausubel et al., Short Protocols in Molecular Biology, 2^{nd} Ed., John Wiley & Sons (1992).

A vector in some embodiments provides for amplification of the copy number of a polynucleotide. A vector can be, for example, an expression vector that provides for expression of an SN protein in a host cell. e.g., a prokaryotic host cell or a eukaryotic host cell.

A polynucleotide or polynucleotides can be contained in a vector or vectors. For example, where a second (or more) nucleic acid molecule is desired, the second nucleic acid molecule can be contained in a vector, which can, but need not be, the same vector as that containing the first nucleic acid molecule. The vector can be any vector useful for introducing a polynucleotide into a genome and can include a nucleotide sequence of genomic DNA (e.g., nuclear or plastid) that is sufficient to undergo homologous recombination with genomic DNA, for example, a nucleotide sequence comprising about 400 to about 1500 or more substantially contiguous nucleotides of genomic DNA.

A regulatory or control element, as the term is used herein, broadly refers to a nucleotide sequence that regulates the transcription or translation of a polynucleotide or the localization of a polypeptide to which it is operatively linked. Examples include, but are not limited to, an RBS, a promoter, enhancer, transcription terminator, an initiation (start) codon, a splicing signal for intron excision and maintenance of a correct reading frame, a STOP codon, an amber or ochre codon, and an IRES. A regulatory element can include a promoter and transcriptional and translational stop signals. Elements may be provided with linkers for the purpose of introducing specific restriction sites facilitating ligation of the control sequences with the coding region of a nucleotide sequence encoding a polypeptide. Additionally, a sequence comprising a cell compartmentalization signal (i.e., a sequence that targets a polypeptide to the cytosol, nucleus, chloroplast membrane or cell membrane) can be attached to the polynucleotide encoding a protein of interest. Such signals are well known in the art and have been widely reported (see, e.g., U.S. Pat. No. 5,776,689).

In a vector, a nucleotide sequence of interest is operably linked to a promoter recognized by the host cell to direct mRNA synthesis. Promoters are untranslated sequences located generally 100 to 1000 base pairs (bp) upstream from the start codon of a structural gene that regulate the transcription and translation of nucleic acid sequences under their control.

Promoters useful for the present disclosure may come from any source (e.g., viral, bacterial, fungal, protist, and animal). The promoters contemplated herein can be specific to photosynthetic organisms, non-vascular photosynthetic organisms, and vascular photosynthetic organisms (e.g., algae, flowering plants). In some instances, the nucleic acids above are inserted into a vector that comprises a promoter of a photosynthetic organism, e.g., algae. The promoter can be a constitutive promoter or an inducible promoter. A promoter typically includes necessary nucleic acid sequences near the start site of transcription, (e.g., a TATA element).

Common promoters used in expression vectors include, but are not limited to, LTR or SV40 promoter, the E. coli lac or trp promoters, and the phage lambda PL promoter. Non-limiting examples of promoters are endogenous promoters such as the psbA and atpA promoter. Other promoters known to control the expression of genes in prokaryotic or eukaryotic cells can be used and are known to those skilled in the art. Expression vectors may also contain a ribosome binding site for translation initiation, and a transcription terminator. The vector may also contain sequences useful for the amplification of gene expression.

A “constitutive” promoter is, for example, a promoter that is active under most environmental and developmental conditions. Constitutive promoters can, for example, maintain a relatively constant level of transcription.

An “inducible” promoter is a promoter that is active under controllable environmental or developmental conditions. For example, inducible promoters are promoters that initiate increased levels of transcription from DNA under their control in response to some change in the environment, e.g. the presence or absence of a nutrient or a change in temperature.

Examples of inducible promoters/regulatory elements include, for example, a nitrate-inducible promoter (for example, as described in Bock et al, Plant Mol. Biol. 17:9 (1991)), or a light-inducible promoter, (for example, as described in Feinbaum et al, Mol Gen. Genet. 226:449 (1991); and Lam and Chua, Science 248:471 (1990)), or a heat responsive promoter (for example, as described in Muller et al., Gene 111: 165-73 (1992)).

In many embodiments, a polynucleotide of the present disclosure includes a nucleotide sequence encoding a protein or enzyme of the present disclosure, where the nucleotide sequence encoding the polypeptide is operably linked to an inducible promoter. Inducible promoters are well known in the art. Suitable inducible promoters include, but are not limited to, the pL of bacteriophage λ; Placo; Ptrp; Ptac (Ptrp-lac hybrid promoter); an isopropyl-beta-D-thiogalactopyranoside (IPTG)-inducible promoter, e.g., a lacZ promoter; a tetracycline-inducible promoter; an arabinose inducible promoter, e.g., P_{BAD} (for example, as described in Guzman et al. (1995) J. Bacteriol. 177:4121-4130); a xylose-inducible promoter, e.g., Pxyl (for example, as described in Kim et al. (1996) Gene 181:71-76); a GAL1 promoter; a tryptophan promoter; a lac promoter; an alcohol-inducible promoter, e.g., a methanol-inducible promoter, an ethanol-inducible promoter; a raffinose-inducible promoter; and a heat-inducible promoter, e.g., heat inducible lambda P_{L} promoter and a promoter controlled by a heat-sensitive repressor (e.g., C1857-repressed lambda-based expression vectors; for example, as described in Hoffmann et al. (1999) FEMS Microbiol Lett. 177(2):327-34).

In many embodiments, a polynucleotide of the present disclosure includes a nucleotide sequence encoding a protein or enzyme of the present disclosure, where the nucleotide sequence encoding the polypeptide is operably linked to a constitutive promoter. Suitable constitutive promoters for use in prokaryotic cells are known in the art and include, but are not limited to, a sigma70 promoter, and a consensus sigma70 promoter.

Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a trc promoter; a tac promoter; an araBAD promoter; in vivo regulated promoters, such as an ssaG promoter or a related promoter (for example, as described in U.S. Patent Publication No. 20040131637), a pagC promoter (for example, as described in Pulkkinen and Miller, J. Bacteriol., 1991: 173(1): 86-93; and Alpuche-Aranda et al., PNAS, 1992; 89(21): 10079-83), a nirB promoter (for example, as described in Harborne et al. (1992) Mol. Micro. 6:2805-2813: Dunstan et al. (1999) Infect. Immun. 67:5133-5141; McKelvie et al. (2004) Vaccine 22:3243-3255; and Chatfield et al. (1992) Biotechnol. 10:888-892): a sigma70 promoter, e.g., a consensus sigma70 promoter (for example, GenBank Accession Nos. AX798980. AX798961, and AX798183); a stationary phase promoter. e.g., a dps promoter, an spy promoter; a promoter derived from the pathogenicity island SPI-2 (for example, as described in WO96/17951); an actA promoter (for example, as described in Shetron-Rama et al. (2002) Infect. Immun. 70:1087-1096); an rpsM promoter (for example, as described in Valdivia and Falkow (1996). Mol. Microbiol. 22:367-378); a tet promoter (for example, as described in Hillen, W. and Wissmann, A. (1989) In Saenger, W, and Heinemann, U. (eds), Topics in Molecular and Structural Biology, Protein-Nucleic Acid Interaction. Macmillan, London, UK, Vol. 10, pp. 143-162); and an SP6 promoter (for example, as described in Melton et al. (1984) Nucl. Acids Res. 12:7035-7056).

In yeast, a number of vectors containing constitutive or inducible promoters may be used. For a review of such vectors see, Current Protocols in Molecular Biology, Vol. 2, 1988, Ed. Ausubel. et al., Greene Publish. Assoc. & Wiley Interscience, Ch. 13: Grant, et al., 1987, Expression and Secretion Vectors for Yeast, in Methods in Enzymology, Eds. Wu & Grossman, 31987, Acad. Press, N.Y., Vol. 153, pp. 516-544: Glover, 1986, DNA Cloning. Vol. II, IRL Press, Wash., D.C., Ch. 3: Bitter, 1987, Heterologous Gene Expression in Yeast, Methods in Enzymology, Eds. Berger & Kimmel, Acad. Press, N.Y., Vol. 152, pp. 673-684; and The Molecular Biology of the Yeast Saccharomyces, 1982, Eds. Strathern et al., Cold Spring Harbor Press, Vols. I and II. A constitutive yeast promoter such as ADH or LEU2 or an inducible promoter such as GAL may be used (for example, as described in Cloning in Yeast, Ch. 3, R. Rothstein In: DNA Cloning Vol. 11, A Practical Approach, Ed. D M Glover, 1986, IRL Press, Wash., D.C.). Alternatively, vectors may be used which promote integration of foreign DNA sequences into the yeast chromosome.

Non-limiting examples of suitable eukaryotic promoters include CMV immediate early, HSV thymidine kinase, early and late SV40, LTRs from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression.

A vector utilized in the practice of the disclosure also can contain one or more additional nucleotide sequences that confer desirable characteristics on the vector, including, for example, sequences such as cloning sites that facilitate manipulation of the vector, regulatory elements that direct replication of the vector or transcription of nucleotide sequences contain therein, and sequences that encode a selectable marker. As such, the vector can contain, for example, one or more cloning sites such as a multiple cloning site, which can, but need not, be positioned such that a exogenous or endogenous polynucleotide can be inserted into the vector and operatively linked to a desired element.

The vector also can contain a prokaryote origin of replication (ori), for example, an E. coli ori or a cosmid ori, thus allowing passage of the vector into a prokaryote host cell, as well as into a plant chloroplast. Various bacterial and viral origins of replication are well known to those skilled in the art and include, but are not limited to the pBR322 plasmid origin, the 2u plasmid origin, and the SV40, polyoma, adenovirus, VSV, and BPV viral origins.

A regulatory or control element, as the term is used herein, broadly refers to a nucleotide sequence that regulates the transcription or translation of a polynucleotide or the localization of a polypeptide to which it is operatively linked. Examples include, but are not limited to, an RBS, a promoter, enhancer, transcription terminator, an initiation (start) codon, a splicing signal for intron excision and maintenance of a correct reading frame, a STOP codon, an amber or ochre codon, an IRES. Additionally, an element can be a cell compartmentalization signal (i.e., a sequence that targets a polypeptide to the cytosol, nucleus, chloroplast membrane or cell membrane). In some aspects of the present disclosure, a cell compartmentalization signal (e.g., a cell membrane targeting sequence) may be ligated to a gene and/or transcript, such that translation of the gene occurs in the chloroplast. In other aspects, a cell compartmentalization signal may be ligated to a gene such that, following translation of the gene, the protein is transported to the cell membrane. Cell compartmentalization signals are well known in the art and have been widely reported (see, e.g., U.S. Pat. No. 5,776,689).

A vector, or a linearized portion thereof, may include a nucleotide sequence encoding a reporter polypeptide or other selectable marker. The term “reporter” or “selectable marker” refers to a polynucleotide (or encoded polypeptide) that confers a detectable phenotype.

A reporter generally encodes a detectable polypeptide, for example, a green fluorescent protein or an enzyme such as luciferase, which, when contacted with an appropriate agent (a particular wavelength of light or luciferin, respectively) generates a signal that can be detected by eye or using appropriate instrumentation (for example, as described in Giacomin, Plant Sci. 116:59-72, 1996; Scikantha, J. Bacteriol. 178:121, 1996: Gerdes, FEBS Lett. 389:44-47, 1996; and Jefferson, EMBO J. 6:3901-3907, 1997, fl-glucuronidase).

A selectable marker (or selectable gene) generally is a molecule that, when present or expressed in a cell, provides a selective advantage (or disadvantage) to the cell containing the marker, for example, the ability to grow in the presence of an agent that otherwise would kill the cell. The selection gene can encode for a protein necessary for the survival or growth of the host cell transformed with the vector.

A selectable marker can provide a means to obtain, for example, prokaryotic cells, eukaryotic cells, and/or plant cells that express the marker and, therefore, can be useful as a component of a vector of the disclosure. The selection gene or marker can encode for a protein necessary for the survival or growth of the host cell transformed with the vector. One class of selectable markers are native or modified genes which restore a biological or physiological function to a host cell (e.g., restores photosynthetic capability or restores a metabolic pathway). Other examples of selectable markers include, but are not limited to, those that confer antimetabolite resistance, for example, dihydrofolate reductase, which confers resistance to methotrexate (for example, as described in Reiss, Plant Physiol. (Life Sci. Adv.) 13:143-149, 1994); neomycin phosphotransferase, which confers resistance to the aminoglycosides neomycin, kanamycin and paromycin (for example, as described in Herrera-Estrella, EMBO J. 2:987-995, 1983), hygro, which confers resistance to hygromycin (for example, as described in Marsh, Gene 32:481-485, 1984), trpB, which allows cells to utilize indole in place of tryptophan; hisD, which allows cells to utilize histinol in place of histidine (for example, as described in Hartman, Proc. Natl. Acad Sci., USA 85:8047, 1988); mannose-6-phosphate isomerase which allows cells to utilize mannose (for example, as described in PCT Publication Application No. WO 94/20627); ornithine decarboxylase, which confers resistance to the ornithine decarboxylase inhibitor, 2-(difluoromethyl)-DL-ornithine (DFMO; for example, as described in McConlogue, 1987, In: Current Communications in Molecular Biology, Cold Spring Harbor Laboratory ed.); and deaminase from Aspergillus terreus, which confers resistance to Blasticidin S (for example, as described in Tamura, Biosci. Biotechnol. Biochem. 59:2336-2338, 1995). Additional selectable markers include those that confer herbicide resistance, for example, phosphinothricin acetyltransferase gene, which confers resistance to phosphinothricin (for example, as described in White et al., Nucl. Acids Res. 18:1062, 1990; and Spencer et al., Theor. Appl. Genet. 79:625-631, 1990), a mutant EPSPV-synthase, which confers glyphosate resistance (for example, as described in Hinchee et al., BioTechnology 91:915-922, 1998), a mutant acetolactate synthase, which confers imidazolione or sulfonylurea resistance (for example, as described in Lee et al., EMBO J. 7:1241-1248, 1988), a mutant psbA, which confers resistance to atrazine (for example, as described in Smeda et al., Plant Physiol. 103:911-917, 1993), or a mutant protoporphyrinogen oxidase (for example, as described in U.S. Pat. No. 5,767,373), or other markers conferring resistance to an herbicide such as glufosinate. Selectable markers include polynucleotides that confer dihydrofolate reductase (DHFR) or neomycin resistance for eukaryotic cells; tetramycin or ampicillin resistance for prokaryotes such as E. coli; and bleomycin, gentamycin, glyphosate, hygromycin, kanamycin, methotrexate, phleomycin, phosphinotricin, spectinomycin, streptomycin, streptomycin, sulfonamide and sulfonylurea resistance in plants (for example, as described in Maliga et al., Methods in Plant Molecular Biology, Cold Spring Harbor Laboratory Press, 1995, page 39). The selection marker can have its own promoter or its expression can be driven by a promoter driving the expression of a polypeptide of interest. The promoter driving expression of the selection marker can be a constitutive or an inducible promoter.

Reporter genes greatly enhance the ability to monitor gene expression in a number of biological organisms. Reporter genes have been successfully used in chloroplasts of higher plants, and high levels of recombinant protein expression have been reported. In addition, reporter genes have been used in the chloroplast of C. reinhardtii. In chloroplasts of higher plants, β-glucuronidase (uidA, for example, as described in Staub and Maliga, EMBO J. 12:601-606, 1993), neomycin phosphotransferase (nptII, for example, as described in Carrer et al., Mol. Gen. Genet. 241:49-56, 1993), adenosyl-3-adenyltransf-erase (aadA, for example, as described in Svab and Maliga, Proc. Natl. Acad. Sci., USA 90:913-917, 1993), and the Aequorea victoria GFP (for example, as described in Sidorov et al., Plant J. 19:209-216, 1999) have been used as reporter genes (for example, as described in Heifetz, Biochemie 82:655-666, 2000). Each of these genes has attributes that make them useful reporters of chloroplast gene expression, such as ease of analysis, sensitivity, or the ability to examine expression in situ. Based upon these studies, other exogenous proteins have been expressed in the chloroplasts of higher plants such as Bacillus thuringiensis Cry toxins, conferring resistance to insect herbivores (for example, as described in Kota et al., Proc. Natl. Acad. Sci., USA 96:1840-1845, 1999), or human somatotropin (for example, as described in Staub et al., Nat. Biotechnol. 18:333-338, 2000), a potential biopharmaceutical. Several reporter genes have been expressed in the chloroplast of the eukaryotic green alga, C. reinhardtii, including aadA (for example, as described in Goldschmidt-Clermont, Nucl. Acids Res. 19:4083-4089 1991; and Zerges and Rochaix, Mol. Cell Biol. 14:5268-5277, 1994), uidA (for example, as described in Sakamoto et al., Proc. Natl. Acad. Sci., USA 90:477-501, 1993; and Ishikura et al., J. Biosci. Bioeng. 87:307-314 1999), Renilla luciferase (for example, as described in Minko et al., Mol. Gen. Genet. 262:421-425, 1999) and the amino glycoside phosphotransferase from Acinetobacter baumanii, aphA6 (for example, as described in Bateman and Purton, Mol. Gen. Genet 263:404-410, 2000).

In one embodiment the protein described herein is modified by the addition of an N-terminal strep-tag epitope to aid in the detection of protein expression. In another embodiment, the protein described herein is modified at the C-terminus by the addition of a Flag-tag epitope to aid in the detection of protein expression, and to facilitate protein purification.

Affinity tags can be appended to proteins so that they can be purified from their crude biological source using an affinity technique. These include, for example, chitin binding protein (CBP), maltose binding protein (MBP), and glutathione-S-transferase (GST). The poly(His) tag is a widely-used protein tag: it binds to metal matrices. Some affinity tags have a dual role as a solubilization agent, such as MBP, and GST. Chromatography tags are used to alter chromatographic properties of the protein to afford different resolution across a particular separation technique. Often, these consist of polyanionic amino acids, such as FLAG-tag. Epitope tags are short peptide sequences which are chosen because high-affinity antibodies can be reliably produced in many different species. These are usually derived from viral genes, which explain their high immunoreactivity. Epitope tags include, but are not limited to, V5-tag, c-myc-tag, and HA-tag. These tags are particularly useful for western blotting and immunoprecipitation experiments, although they also find use in antibody purification.

Fluorescence tags are used to give visual readout on a protein. GFP and its variants are the most commonly used fluorescence tags. More advanced applications of GFP include using it as a folding reporter (fluorescent if folded, colorless if not).

In one embodiment, the proteins described herein can be fused at the amino-terminus to the carboxy-terminus of a highly expressed protein (fusion partner). These fusion partners may enhance the expression of the gene. Engineered processing sites, for example, protease, proteolytic, or tryptic processing or cleavage sites, can be used to liberate the protein from the fusion partner, allowing for the purification of the intended protein. Examples of fusion partners that can be fused to the gene are a sequence encoding the mammary-associated serum amyloid (M-SAA) protein, a sequence encoding the large and/or small subunit of ribulose bisphosphate carboxylase, a sequence encoding the glutathione S-transferase (GST) gene, a sequence encoding a thioredoxin (TRX) protein, a sequence encoding a maltose-binding protein (MBP), a sequence encoding any one or more of E. coli proteins NusA, NusB, NusG, or NusE, a sequence encoding a ubiqutin (Ub) protein, a sequence encoding a small ubiquitin-related modifier (SUMO) protein, a sequence encoding a cholera toxin B subunit (CTB) protein, a sequence of consecutive histidine residues linked to the 3′ end of a sequence encoding the MBP-encoding malE gene, the promoter and leader sequence of a galactokinase gene, and the leader sequence of the ampicillinase gene.

In some instances, the vectors of the present disclosure will contain elements such as an E. coli or S. cerevisiae origin of replication. Such features, combined with appropriate selectable markers, allows for the vector to be “shuttled” between the target host cell and a bacterial and/or yeast cell. The ability to passage a shuttle vector of the disclosure in a secondary host may allow for more convenient manipulation of the features of the vector. For example, a reaction mixture containing the vector and inserted polynucleotide(s) of interest can be transformed into prokaryote host cells such as E. coli, amplified and collected using routine methods, and examined to identify vectors containing an insert or construct of interest. If desired, the vector can be further manipulated, for example, by performing site directed mutagenesis of the inserted polynucleotide, then again amplifying and selecting vectors having a mutated polynucleotide of interest. A shuttle vector then can be introduced into plant cell chloroplasts, wherein a polypeptide of interest can be expressed and, if desired, isolated according to a method of the disclosure.

Knowledge of the chloroplast or nuclear genome of the host organism, for example, C. reinhardtii, is useful in the construction of vectors for use in the disclosed embodiments. Chloroplast vectors and methods for selecting regions of a chloroplast genome for use as a vector are well known (see, for example, Bock, J. Mol. Biol. 312:425-438, 2001: Staub and Maliga, Plant Cell 4:39-45, 1992; and Kavanagh et al., Genetics 152:1111-1122, 1999, each of which is incorporated herein by reference). The entire chloroplast genome of C. reinhardtii is available to the public on the world wide web, at the URL “biology.duke.edu/chlamy_genome/-chloro.html” (see “view complete genome as text file” link and “maps of the chloroplast genome” link; J. Maul. J. W. Lilly, and D. B. Stem, unpublished results; revised Jan. 28, 2002: to be published as GenBank Ace. No. AF396929; and Maul. J. E., et al. (2002) The Plant Cell, Vol. 14 (2659-2679)). Generally, the nucleotide sequence of the chloroplast genomic DNA that is selected for use is not a portion of a gene, including a regulatory sequence or coding sequence. For example, the selected sequence is not a gene that if disrupted, due to the homologous recombination event, would produce a deleterious effect with respect to the chloroplast. For example, a deleterious effect on the replication of the chloroplast genome or to a plant cell containing the chloroplast.

In this respect, the website containing the C. reinhardtii chloroplast genome sequence also provides maps showing coding and non-coding regions of the chloroplast genome, thus facilitating selection of a sequence useful for constructing a vector (also described in Maul, J. E., et al. (2002) The Plant Cell, Vol. 14 (2659-2679)). For example, the chloroplast vector, p322, is a clone extending from the Eco (Eco RI) site at about position 143.1 kb to the Xho (Xho I) site at about position 148.5 kb (see, world wide web, at the URL “biology.duke.edu/chlamy_genome/chloro.html”, and clicking on “maps of the chloroplast genome” link, and “140-150 kb” link; also accessible directly on world wide web at URL “biology.duke.edu/chlam-y/chloro/chlorol40.html”).

In addition, the entire nuclear genome of C. reinhardtii is described in Merchant, S. S., et al., Science (2007), 318(5848):245-250, thus facilitating one of skill in the art to select a sequence or sequences useful for constructing a vector.

For expression of the polypeptide in a host, an expression cassette or vector may be employed. The expression vector will comprise a transcriptional and translational initiation region, which may be inducible or constitutive, where the coding region is operably linked under the transcriptional control of the transcriptional initiation region, and a transcriptional and translational termination region. These control regions may be native to the gene, or may be derived from an exogenous source. Expression vectors generally have convenient restriction sites located near the promoter sequence to provide for the insertion of nucleic acid sequences encoding exogenous or endogenous proteins. A selectable marker operative in the expression host may be present.

The nucleotide sequences may be inserted into a vector by a variety of methods. In the most common method the sequences are inserted into an appropriate restriction endonuclease site(s) using procedures commonly known to those skilled in the art and detailed in, for example, Sambrook et al., Molecular Cloning, A Laboratory Manual, 2^{nd} Ed., Cold Spring Harbor Press, (1989) and Ausubel et al., Short Protocols in Molecular Biology, 2^{nd} Ed., John Wiley & Sons (1992).

The description herein provides that host cells may be transformed with vectors. One of skill in the art will recognize that such transformation includes transformation with circular vectors, linearized vectors, linearized portions of a vector, or any combination of the above.

Thus, a host cell comprising a vector may contain the entire vector in the cell (in either circular or linear form), or may contain a linearized portion of a vector of the present disclosure.

Codon Optimization

One or more codons of an encoding polynucleotide can be “biased” or “optimized” to reflect the codon usage of the host organism. These two terms can be used interchangeably throughout the disclosure. For example, one or more codons of an encoding polynucleotide can be “biased” or “optimized” to reflect chloroplast codon usage (Table A) or nuclear codon usage (Table B) in Chlamydomonas reinhardtii. Most amino acids are encoded by two or more different (degenerate) codons, and it is well recognized that various organisms utilize certain codons in preference to others. Generally, the codon bias selected reflects codon usage of the plant (or organelle therein) which is being transformed with the nucleic acid or acids of the present disclosure. However, the codon bias need not be selected based on a particular organism in which a polynucleotide is to be expressed.

One or more codons can be modified, for example, by a method such as site directed mutagenesis, PCR using a primer that is mismatched for the nucleotide(s) to be changed such that the amplification product is biased to reflect the selected (chloroplast or nuclear) codon usage, or by the de novo synthesis of a polynucleotide sequence such that the change (bias) is introduced as a consequence of the synthesis procedure.

When codon-optimizing a specific gene sequence for expression, factors other than the codon usage may also be taken into consideration. For example, it is typical to avoid restrictions sites, repeat sequences, and potential methylation sites. Most gene synthesis companies utilize computational algorithms to optimize a DNA sequence taking into consideration these and other factors whilst maintaining the codon usage (as defined in the codon usage table) above a user-defined threshold. For example, this threshold may be set such that a codon that is used less than 10% of the time that the corresponding amino acid is present in the proteome would be avoided in the final DNA sequence.

Table A (below) shows the chloroplast codon usage for C. reinhardtii (see U.S. Patent Application Publication No.: 2004/0014174, published Jan. 22, 2004).

TABLE A
Chloroplast Codon Usage in Chlamydomonas reinhardtii

| | | | |
|---|---|---|---|
| UUU 34.1*(348**) | UCU 19.4(198) | UAU 23.7(242) | UGU 8.5(87) |
| UUC 14.2(145) | UCC 4.9(50) | UAC 10.4(106) | UGC 2.6(27) |
| UUA 72.8(742) | UCA 20.4(208) | UAA 2.7(28) | UGA 0.1(1) |
| UUG 5.6(57) | UCG 5.2(53) | UAG 0.7(7) | UGG 13.7(140) |
| CUU 14.8(151) | CCU 14.9(152) | CAU 11.1(113) | CGU 25.5(260) |
| CUC 1.0(10) | CCC 5.4(55) | CAC 8.4(86) | CGC 5.1(52) |
| CUA 6.8(69) | CCA 19.3(197) | CAA 34.8(355) | CGA 3.8(39) |
| CUG 7.2(73) | CCG 3.0(31) | CAG 5.4(55) | CGG 0.5(5) |
| AUU 44.6(455) | ACU 23.3(237) | AAU 44.0(449) | AGU 16.9(172) |
| AUC 9.7(99) | ACC 7.8(80) | AAC 19.7(201) | AGC 6.7(68) |
| AUA 8.2(84) | ACA 29.3(299) | AAA 61.5(627) | AGA 5.0(51) |
| AUG 23.3(238) | ACG 4.2(43) | AAG 11.0(112) | AGG 1.5(15) |
| GUU 27.5(280) | GCU 30.6(312) | GAU 23.8(243) | GGU 40.0(408) |
| GUC 4.6(47) | GCC 11.1(113) | GAC 11.6(118) | GGC 8.7(89) |
| GUA 26.4(269) | GCA 19.9(203) | GAA 40.3(411) | GGA 9.6(98) |
| GUG 7.1(72) | GCG 4.3(44) | GAG 6.9(70) | GGG 4.3(44) |

*Frequency of codon usage per 1,000 codons.
**Number of times observed in 36 chloroplast coding sequences (10,193 codons).

The C. reinhardtii chloroplast genome shows a high AT content and noted codon bias (for example, as described in Franklin S., et al. (2002) Plant J 30:733-744; Mayfield S. P, and Schultz J. (2004) Plant J 37:449-458).

Table B exemplifies codons that are preferentially used in Chlamydomonas nuclear genes.

TABLE B
fields: [triplet] [frequency: per thousand] ([number])
Coding GC 66.30% 1^{st} letter GC 64.80% 2^{nd} letter GC 47.90% 3^{rd} letter GC 86.21%
Nuclear Codon Usage in Chlamydomonas reinhardtii

| | | | |
|---|---|---|---|
| UUU 5.0 (2110) | UCU 4.7 (1992) | UAU 2.6 (1085) | UGU 1.4 (601) |
| UUC 27.1 (11411) | UCC 16.1 (6782) | UAC 22.8 (9579) | UGC 13.1 (5498) |
| UUA 0.6 (247) | UCA 3.2 (1348) | UAA 1.0 (441) | UGA 0.5 (227) |
| UUG 4.0 (1673) | UCG 16.1 (6763) | UAG 0.4 (183) | UGG 13.2 (5559) |
| CUU 4.4 (1869) | CCU 8.1 (3416) | CAU 2.2 (919) | CGU 4.9 (2071) |
| CUC 13.0 (5480) | CCC 29.5 (12409) | CAC 17.2 (7252) | CGC 34.9 (14676) |
| CUA 2.6 (1086) | CCA 5.1 (2124) | CAA 4.2 (1780) | CGA 2.0 (841) |
| CUG 65.2 (27420) | CCG 20.7 (8684) | CAG 36.3 (15283) | CGG 11.2 (4711) |
| AUU 8.0 (3360) | ACU 5.2 (2171) | AAU 2.8 (1157) | AGU 2.6 (1089) |
| AUC 26.6 (11200) | ACC 27.7 (11663) | AAC 28.5 (11977) | AGC 22.8 (9590) |
| AUA 1.1 (443) | ACA 4.1 (1713) | AAA 2.4 (1028) | AGA 0.7 (287) |
| AUG 25.7 (10796) | ACG 15.9 (6684) | AAG 43.3 (18212) | AGG 2.7 (1150) |
| GUU 5.1 (2158) | GCU 16.7 (7030) | GAU 6.7 (2805) | GGU 9.5 (3984) |
| GUC 15.4 (6496) | GCC 54.6 (22960) | GAC 41.7 (17519) | GGC 62.0 (26064) |
| GUA 2.0 (857) | GCA 10.6 (4467) | GAA 2.8 (1172) | GGA 5.0 (2084) |
| GUG 46.5 (19558) | GCG 44.4 (18688) | GAG 53.5 (22486) | GGG 9.7 (4087) |

Generally, the nuclear codon bias selected for purposes of the present disclosure, including, for example, in preparing a synthetic polynucleotide as disclosed herein, can reflect nuclear codon usage of an algal nucleus and includes a codon bias that results in the coding sequence containing greater than 60% G/C content.

Re-Engineering the Genome.

In addition to utilizing codon bias as a means to provide efficient translation of a polypeptide, it will be recognized that an alternative means for obtaining efficient translation of a polypeptide in an organism is to re-engineer the genome (e.g., a C. reinhardtii chloroplast or nuclear genome) for the expression of tRNAs not otherwise expressed in the genome. Such an engineered algae expressing one or more exogenous tRNA molecules provides the advantage that it would obviate a requirement to modify every polynucleotide of interest that is to be introduced into and expressed from an algal genome; instead, algae such as C. reinhardtii that comprise a genetically modified genome can be provided and utilized for efficient translation of a polypeptide. Correlations between tRNA abundance and codon usage in highly expressed genes is well known (for example, as described in Franklin et al., Plant J. 30:733-744, 2002; Dong et al., J. Mol. Biol. 260:649-663, 1996; Duret, Trends Genet. 16:287-289, 2000; Goldman et. al., J. Mol. Biol. 245:467-473, 1995; and Komar et. al., Biol. Chem. 379:1295-1300, 1998). In E. coli, for example, re-engineering of strains to express underutilized tRNAs resulted in enhanced expression of genes which utilize these codons (see Novy et al., in Novations 12:1-3, 2001). Utilizing endogenous tRNA genes, site directed mutagenesis can be used to make a synthetic tRNA gene, which can be introduced into the genome of the host organism to complement rare or unused tRNA genes in the genome, such as a C. reinhardtii chloroplast genome.

Another Way to Codon Optimize a Sequence for Expression.

An alternative way to optimize a nucleic acid sequence for expression is to use the most frequently utilized codon (as determined by a codon usage table) for each amino acid position. This type of optimization may be referred to as ‘hot codon’ optimization. Should undesirable restriction sites be created by such a method then the next most frequently utilized codon may be substituted in a position such that the restriction site is no longer present. Table C lists the codon that would be selected for each amino acid when using this method for optimizing a nucleic acid sequence for expression in the chloroplast of C. reinhardtii.

TABLE C

| Amino acid | Codon utilized |
|---|---|
| F | TTC |
| L | TTA |
| I | ATC |
| V | GTA |
| S | TCA |
| P | CCA |
| T | ACA |
| A | GCA |
| Y | TAC |
| H | CAC |
| Q | CAA |
| N | AAC |
| K | AAA |
| D | GAC |
| E | GAA |
| C | TGC |
| R | CGT |
| G | GGC |
| W | TGG |
| M | ATG |
| STOP | TAA |

Codon Optimization for the Nucleus of a Desmodesmus, Chlamydomonas, Nannochloropsis, or Scenedesmus Species.

To create a codon usage table that can be used to express a gene in the nucleus of several different species, the codon usage frequency of a number of species were analyzed. 30,000 base pairs of DNA sequence corresponding to nuclear protein coding regions for the each of the algal species Scenedesmus sp. (S. dimorphus), Desmodesmus sp. (an unknown Desmodesmus sp.), and Nannochloropsis sp. (N. salina) were used to create a unique nuclear codon usage table for each species. These tables were then compared to each other and to that of Chlamydomonas reinhardtii; the codon table for the nuclear genome of Chlamydomonas reinhardii was used as a standard. Any codons that had very low codon usage for the other algal species but not in Chlamydomonas reinhardii were fixed at 0 and thus should be avoided in a DNA sequence designed using this codon table (Table D). The following codons should be avoided CGG, CAT, CCG, and TCG. The codon usage table generated is shown in Table D.

TABLE D
Nuclear Codon usage in a Chlamydomonas sp., Scenedesmus sp.,
Desmodesmus sp., and Nannochloropsis sp.
For example, in the first row, the fraction (0.16) is the percentage (16%)
of times that a codon (UUU) is used to code for F (phenylalanine).

| Triplet | a.a. | Fraction | Triplet | a.a. | Fraction | Triplet | a.a. | Fraction | Triplet | a.a. | Fraction |
|---|---|---|---|---|---|---|---|---|---|---|---|
| UUU | F | 0.16 | UCU | S | 0.1 | UAU | Y | 0.1 | UGU | C | 0.1 |
| UUC | F | 0.84 | UCC | S | 0.33 | UAC | Y | 0.9 | UGC | C | 0.9 |
| UUA | L | 0.01 | UCA | S | 0.06 | UAA | * | 0.52 | UGA | * | 0.27 |
| UUG | L | 0.04 | UCG | S | 0 | UAG | * | 0.22 | UGG | W | 1 |
| CUU | L | 0.05 | CCU | P | 0.19 | CAU | H | 0 | CGU | R | 0.11 |
| CUC | L | 0.15 | CCC | P | 0.69 | CAC | H | 1 | CGC | R | 0.77 |
| CUA | L | 0.03 | CCA | P | 0.12 | CAA | Q | 0.1 | CGA | R | 0.04 |
| CUG | L | 0.73 | CCG | P | 0 | CAG | Q | 0.9 | CGG | R | 0 |
| AUU | I | 0.22 | ACU | T | 0.1 | AAU | N | 0.09 | AGU | S | 0.05 |
| AUC | I | 0.75 | ACC | T | 0.52 | AAC | N | 0.91 | AGC | S | 0.46 |
| AUA | I | 0.03 | ACA | T | 0.08 | AAA | K | 0.05 | AGA | R | 0.02 |
| AUG | M | 1 | ACG | T | 0.3 | AAG | K | 0.95 | AGG | R | 0.06 |
| GUU | V | 0.07 | GCU | A | 0.13 | GAU | D | 0.14 | GGU | G | 0.11 |
| GUC | V | 0.22 | GCC | A | 0.43 | GAC | D | 0.86 | GGC | G | 0.72 |
| GUA | V | 0.03 | GCA | A | 0.08 | GAA | E | 0.05 | GGA | G | 0.06 |
| GUG | V | 0.67 | GCG | A | 0.35 | GAG | E | 0.95 | GGG | G | 0.11 |

(* represents stop codons)
(a.a. is amino acid)

Percent Sequence Identity

One example of an algorithm that is suitable for determining percent sequence identity or sequence similarity between nucleic acid or polypeptide sequences is the BLAST algorithm, which is described, e.g., in Altschul et al., J. Mol. Biol. 215:403-410 (1990). Software for performing BLAST analysis is publicly available through the National Center for Biotechnology Information. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (as described, for example, in Henikoff & Henikoff (1989) Proc. Natl. Acad. Sci. USA, 89:10915). In addition to calculating percent sequence identity, the BLAST algorithm also can perform a statistical analysis of the similarity between two sequences (for example, as described in Karlin & Altschul. Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, less than about 0.01, or less than about 0.001.

General Lipid Classes

A lipid is defined herein as a cellular component that is not soluble in water and is soluble in a non-polar solvent. Examples of lipids are acyl lipids, isoprenoids, porphyrins, or a cellular component that is derived from an acyl lipid.

Other exemplary lipids include a heme, a polar lipid, a chlorophyll breakdown product, pheophytin, a digalactosyl diacylglycerol (DGDG), a triacylglycerol, a diacylglycerol, a monoacylglycerol, a sterol, a sterol ester, a wax ester, a tocopherol, a fatty acid, phosphatidic acid, lysophosphatidic acid, phosphatidyl glycerol, cardiolipin (diphosphatidylglycerol), phosphatidyl choline, lysophospatidyl choline, phosphatidyl ethanolamine, phosphatidyl serine, phosphatidylinositol, phosphonyl ethanolamine, an ether lipid, monogalactosyl diacylglycerol, digalactosyl diacylglycerol, sulfoquinovosyl diacylglycerol, sphingosine, phytosphingosine, sphingomyelin, glucosylceramide, diacylglyceryl trimethylhomoserine, ricinoleic acid, prostaglandin, jasmonic acid, a-Carotene, b-Carotene, b-cryptoxanthin, astaxanthin, zeaxanthin, chlorophyll a, chlorophyll b, pheophytin a, phylloquinone, plastoquinone, chlorophyllide a, chlorophillide b, pheophorbide a, pyropheophorbide a, pheophorbide b, pheophytin b, hydroxychlorophyll a, hydroxypheophytin a, methoxylactone chlorophyll a, pyrochlorophillide a, pyropheophytin a, diacylglyceryl glucuronide, diacylglyceryl OH methyl carboxy choline, diacylglyceryl OH methyl trimethyl alanine, 2′-O-acyl-sulfoquinovosyldiacylglycerol, phosphatidylinositol-4-phosphate, or phosphatidylinositol-4,5-bisphosphate.

“Content” is the total amount of any one or more of the above-mentioned lipids. A “profile” is the relative amount of any one or more of the above-mentioned lipids.

For example, a transformed organism's lipid content can be different than that of an untransformed organism's lipid content in that expression of a particular lipid is increased in the transformed organism as compared to the untransformed organism therefore increasing the total amount of lipid in the organism.

Also, for example, a transformed organism's lipid profile can be different than that of an untransformed organism's lipid profile in that expression of several lipids are either increased or decreased in the transformed organism as compared to the untransformed organism.

A transformed organism's lipid content or profile can also be compared to any other organism, for example, another transformed organism.

### EXAMPLES

The following examples are intended to provide illustrations of the application of the present disclosure. The following examples are not intended to completely define or otherwise limit the scope of the disclosure.

One of skill in the art will appreciate that many other methods known in the art may be substituted in lieu of the ones specifically described or referenced herein.

Several of the methods described below have been previously described in U.S. Provisional Patent Application No. 61/301,141 filed Feb. 3, 2010, and International Publication No. WO 2011/097261, with an international filing date of Feb. 1, 2011 and published on Aug. 11, 2011.

### Example 1

### Nitrogen Starvation Phenotypes in Wild Type Algae

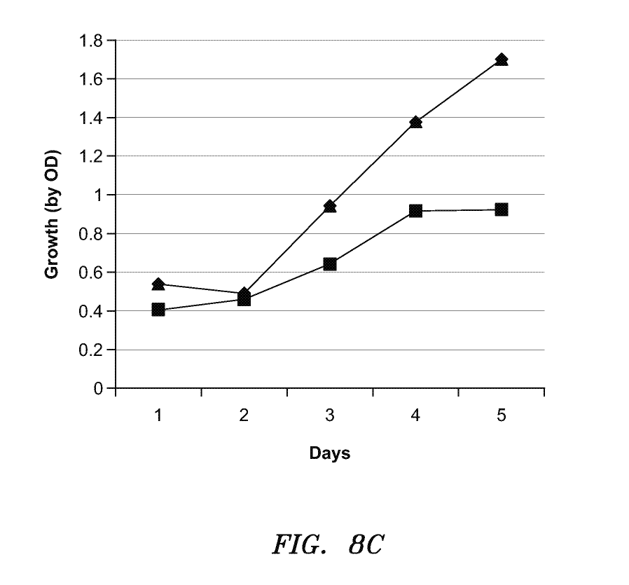

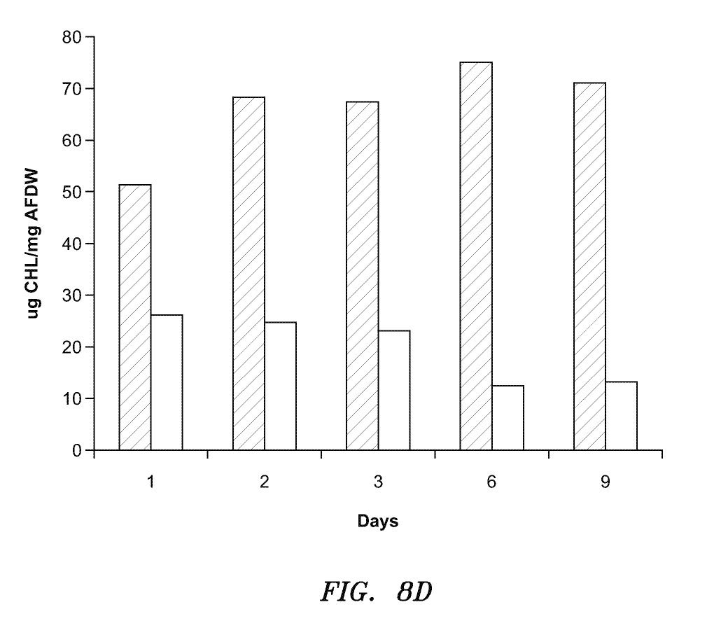

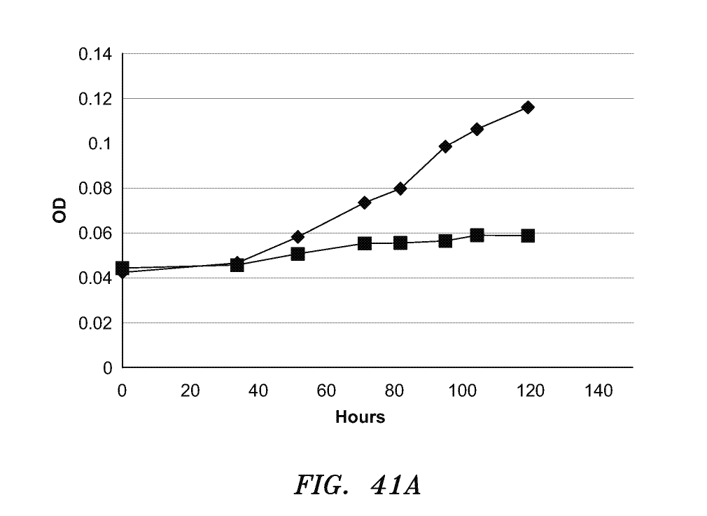

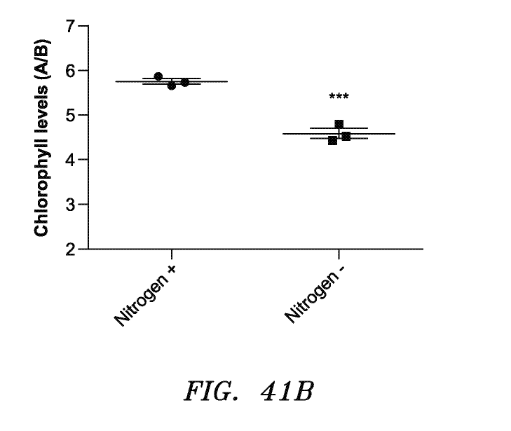

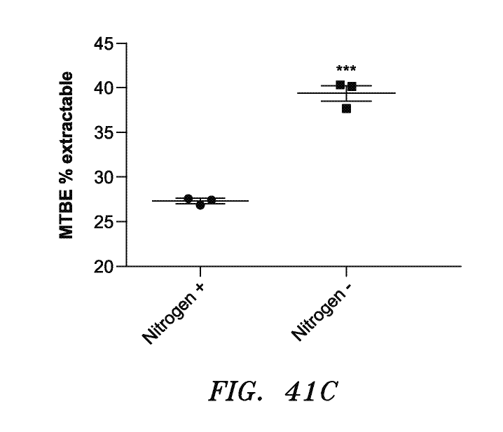

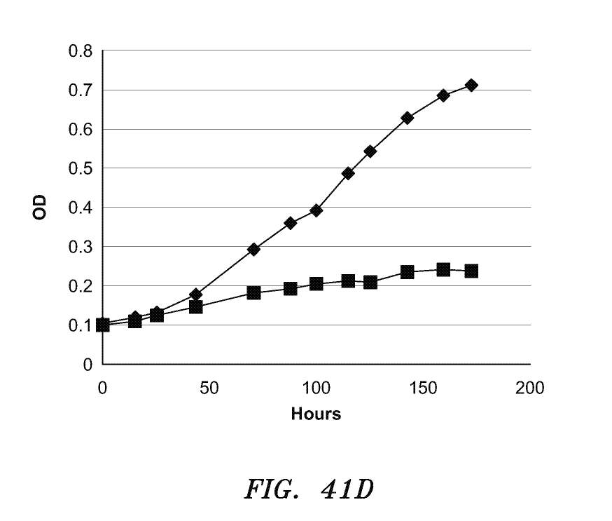

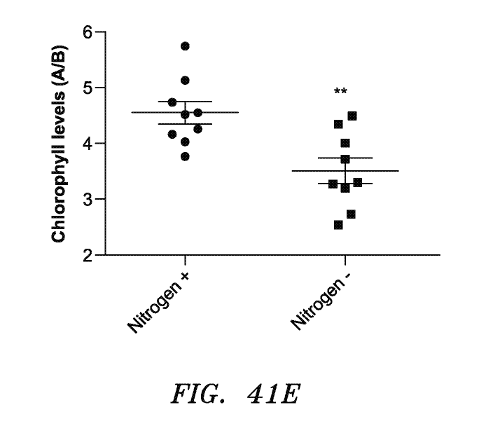

Nitrogen starvation in many wild type algae species (for example, Dunaliella salina, Scenedesmus dimorphus, Dunaliella viridis, Chlamydomonas reinhardtii and Nannochloropsis salina) is known to cause several phenotypes, among them an increase in total lipids (FIGS. 8A and 8B, FIG. 41C), reduced growth (FIG. 8C, FIGS. 41A and 41D), and a breakdown of chlorophyll (FIG. 8D and FIGS. 41B and 41E). It would be desirable to separate these phenotypic pathways at the molecular level. For example, it would be desirable to obtain an increased lipid phenotype that does not have decreased growth and the breakdown of algal components.

FIG. 8A shows gravimetric fats analyses (hexane extractables). The left hand column of each group of two is percent lipids by hexane extractable (% DW) after growth in minimal media containing 7.5 mM NH4Cl, and the right hand column of each group of two is percent lipids by hexane extractable (% DW) after growth in minimal media in the absence of nitrogen. Three different strains are identified: SE0004 (Scenedesmus dimorphus). SE0043 (Dunaliella viridis) and SE0050 (Chlamydomas reinhardtii). These strains represent three different orders of the Class Chlorophyceae.

FIG. 8B shows gravimetric fats analyses (hexane extractables). The left hand column of each group of two is percent lipids by hexane extractable (% DW) after growth in minimal media containing 7.5 mM NH4Cl, and the right hand column of each group of two is percent lipids by hexane extractable (% DW) after growth in minimal media in the absence of nitrogen. Three different strains are identified: SE0003 (Dunaliella salina), SE0004 (Scenedesmus dimorphus) and SE0043 (Dunaliella viridis). These strains represent three different orders of the Class Chlorophyceae.

FIG. 41C shows extractable lipid in algae grown under nitrogen stress. Wild type Nannochloropsis salina was grown in MASM containing 11.8 mM NaNO3, 0.5 mM NH4Cl and 16 ppt NaCl in a 5% carbon dioxide in an air environment under constant light to early log phase. 2-3 L of the culture was centrifuged at 3000 to 5000×g for 5-10 minutes and one half of the culture was washed with 300-500 mL MASM, the other half with 300-500 mL MASM containing no nitrogen. After re-centrifugation, the two cultures were resuspended in a volume of media (MASM or MASM containing no nitrogen) equivalent to the starting culture volume. After two days, samples were collected and centrifuged. The cells were analyzed for total gravimetric lipids by methanolimethyl-tert-butyl ether extraction according to a modified Bligh Dyer method (as described in Matyash V., et al. (2008) Journal of Lipid Research 49:1137-1146). The percent extractable is shown on the y axis and the sample in the presence and absence of nitrogen are indicated on the x axis.

FIG. 8C shows algal growth under nitrogen stress. Chlamydomonas reinhardtii wild type was grown in 50-100 mL HSM containing 7.5 mM NH4Cl in a 5% carbon dioxide in an air environment under constant light to early log phase. The culture was centrifuged at 3000 to 5000×g for 5-10 minutes and one half of the culture was washed with 20-50 mL of HSM, the other half with 20-50 mL HSM containing no nitrogen. After re-centrifugation, the two cultures were resuspended in a volume of media (HSM or HSM containing no nitrogen) equivalent to the starting culture volume. This point was recorded as day 0. Optical density (OD) as 750 nm was taken each day over a time course of 5 days and is shown on the y axis. The x-axis represents the time course of nitrogen starvation over 5 days. The triangle represents growth in the presence of nitrogen and the square represents growth in the absence of nitrogen.

FIG. 41A shows growth of Nannochloropsis salina under nitrogen stress. Wild type Nannochloropsis salina was grown in 50-100 mL of MASM containing 11.8 mM NaNO3, 0.5 mM NH4Cl and 16 ppt NaCl in a 5% carbon dioxide in an air environment under constant light to early log phase. The culture was centrifuged at 3000 to 5000×g for 5-10 minutes and one half of the culture was washed with 20-50 mL of MASM, the other half with 20-50 mL of MASM containing no nitrogen. After re-centrifugation, the two cultures were resuspended in a volume of media (MASM or MASM containing no nitrogen) equivalent to the starting culture volume. This point was recorded as time 0. Optical density (OD) as 750 nm was taken each day over a time course of 120 hours and is shown on the y axis. The x-axis represents the time course of nitrogen starvation over 5 days. The diamond represents growth in the presence of nitrogen and the square represents growth in the absence of nitrogen.

FIG. 41D shows growth of Scenedesmus dimorphus under nitrogen stress. Wild type Scenedesmus dimorphus was grown in 50-100 mL of HSM containing 7.5 mM NH4Cl in a 5% carbon dioxide in an air environment under constant light to early log phase. The culture was centrifuged at 3000 to 5000×g for 5-10 minutes and one half of the culture was washed with 30-50 mL of HSM, the other half with 20-50 mL of HSM containing no nitrogen. After re-centrifugation, the two cultures were resuspended in a volume of media (HSM or HSM containing no nitrogen) equivalent to the starting culture volume. This point was recorded as time 0. Optical density (OD) as 750 nm was taken 1-2 times a day over a time course of 180 hours and is shown on the y axis. The x-axis represents the time course of nitrogen starvation over 7.5 days. The diamond represents growth in the presence of nitrogen and the square represents growth in the absence of nitrogen.

FIG. 8D shows chlorophyll (μg chlorophyll/mg ash free dry weight (AFDW)) under nitrogen stress. Chlamydomonas reinhardtii wild type was grown in 50-100 mL HSM containing 7.5 mM NH4Cl in a 5% carbon dioxide in an air environment under constant light to early log phase. The culture was centrifuged at 3000 to 5000×g for 5-10 minutes and one half of the culture was washed with 20-50 mL HSM, the other half with 20-50 mL HSM containing no nitrogen. After re-centrifugation, the two cultures were resuspended in a volume of media (HSM or HSM containing no nitrogen) equivalent to the starting culture volume. This point was recorded as day 0. Samples were collected and centrifuged. Cells were extracted in methanol and chlorophyll levels were determined spectroscopically as described in (LICHTENTHALER. Chlorophylls and Carotenoids: Pigments of Photosynthetic Biomembranes. Meth Enzymol (1987) vol. 148 pp. 350-382). Optical density (OD) of the culture at 750 nm was used to normalize to cell density and to approximate AFDW. Measurements were taken over a time course of 9 days. The left hand column of each group of two is chlorophyll content in the presence of nitrogen and the right hand column of each group of two is chlorophyll content in the absence of nitrogen.

FIG. 41B shows chlorophyll levels under nitrogen stress. Wild type Nannochloropsis salina was grown in 50-100 mL of MASM containing 11.8 mM NaNO3, 0.5 mM NH4Cl and 16 ppt NaCl in a 5% carbon dioxide in an air environment under constant light to early log phase. The culture was centrifuged at 3000 to 5000×g for 5-10 minutes and one half of the culture was washed with 20-50 mL MASM, the other half with 20-50 mL MASM containing no nitrogen. After re-centrifugation, the two cultures were resuspended in a volume of media (MASM or MASM containing no nitrogen) equivalent to the starting culture volume. After two days, samples were collected and centrifuged. Cells were extracted in methanol and chlorophyll levels we determined spectroscopically as described in (LICHTENTHALER. Chlorophylls and Carotenoids: Pigments of Photosynthetic Biomembranes. Meth Enzymol (1987) vol. 148 pp. 350-382). Calculations of chlorophyll A and chlorophyll B were added and optical density (OD) of the culture at 750 nm was used to normalize to cell density. This value is plotted on the y axis and the sample in the presence and absence of nitrogen are indicated on the x axis.

FIG. 41E shows chlorophyll levels under nitrogen stress. Wild type Scenedesmus dimorphus was grown in 50-100 mL of HSM containing 7.5 mM NH4Cl in a 5% carbon dioxide in an air environment under constant light to early log phase. The culture was centrifuged at 3000 to 5000×g for 5-10 minutes and one half of the culture was washed with 20-50 mL HSM, the other half with 20-50 mL HSM containing no nitrogen. After re-centrifugation, the two cultures were resuspended in a volume of media (HSM or HSM containing no nitrogen) equivalent to the starting culture volume. After two days, samples were collected and centrifuged. Cells were extracted in methanol and chlorophyll levels we determined spectroscopically as described in (LICHTENTHALER. Chlorophylls and Carotenoids: Pigments of Photosynthetic Biomembranes. Meth Enzymol (1987) vol. 148 pp. 350-382). Calculations of chlorophyll A and chlorophyll B were added and optical density (OD) of the culture at 750 nm was used to normalize to cell density. This value is plotted on the y axis and the sample in the presence and absence of nitrogen are indicated on the x axis.

### Example 2

### Timing of the Stress Response in Wild Type Chlamydomonas reinhardtii at the Biochemical and Molecular Level

In this example, the timing of the biochemical and molecular responses of wild type Chlamydomonas reinhardtii was investigated. Wild-type Chlamydomonas reinhardtii cells were grown in 5-10 L of HSM media in a 5% carbon dioxide in an air environment under constant light, until cells reached early log phase. The culture was centrifuged at 3000 to 5000×g for 5-10 minutes and one half of the culture was washed with 500-1000 mL HSM, the other half with 500-1000 mL HSM containing no nitrogen. After re-centrifugation, the two cultures were resuspended in a volume of media (HSM or HSM containing no nitrogen) equivalent to the starting culture volume. At the time points listed in Table 2, 0.5-2 L of the cells were harvested by centrifugation and analyzed for total gravimetric lipids by the Bligh Dyer method (as described in BLIGH and DYER, A rapid method of total lipid extraction and purification. Can J Biochem Physiol (1959) vol. 37 (8) pp. 911-7). The percent extractables was calculated using the ash free dry weight of the sample.

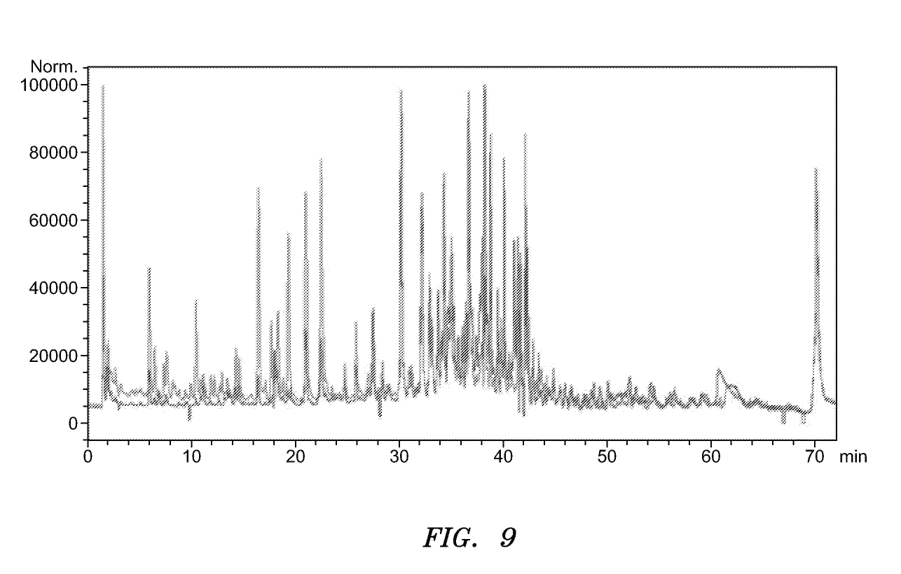

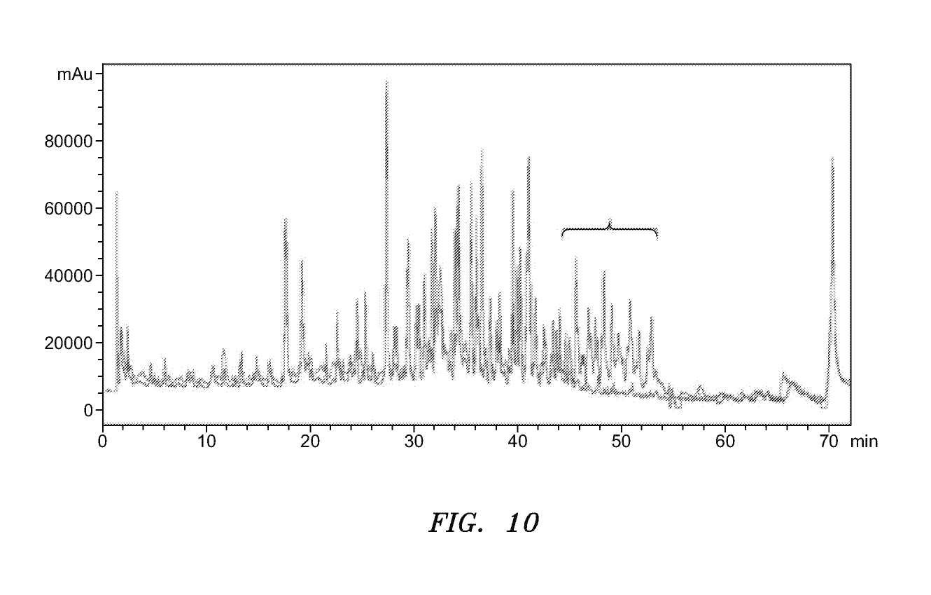

Bligh-Dyer extracted oils from SE0050 were run on reverse-phase HPLC on a C18 column. Mobile phase A was MeOH/water/HOAc (750:250:4). Mobile phase B was CAN/MeOH/THF/HOAc (500:375:125:4) with a gradient between A and B over 72 minutes and flow rate of 0.8 mL/min. Detection was via a Charged Aerosol Detector (CAD). Differences in the lipid phenotype of SE0050 were observed at 24 and 48 hours after nitrogen starvation. This assay is a qualitative assay for total lipid profile in nitrogen replete and nitrogen starved conditions. The y-axis is the CAD signal which represents abundance and the x axis is HPLC column retention time (in minutes). As shown in FIG. 9, some minor differences (in the lipid profile) are seen at the 24 hour time point. In contrast, a major shift (as shown in FIG. 10) is seen 48 hours after the removal of nitrogen from the HSM media. TAGs are detected between 44 and 54 minutes retention time, demonstrating that there is a large increase in TAGs by 48 hours of nitrogen starvation. These differences indicate that the lipid phenotype is seen (in this strain under this starvation regime) between 24 and 48 hours after nitrogen starvation.

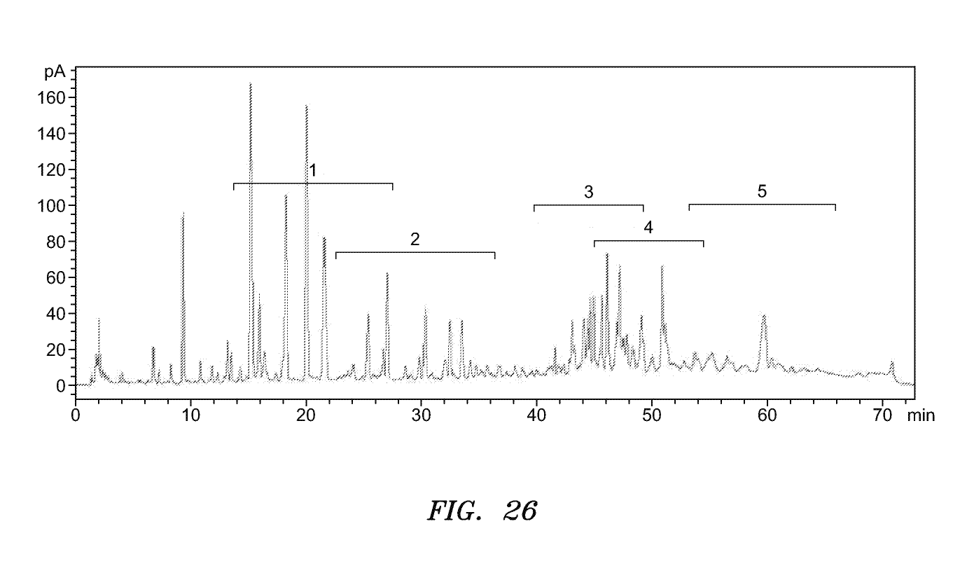

FIG. 26 shows a reference trace for an algal hexane extract on HPLC/CAD as produced by the CAD vendor (ESA—A Dionex Company). This reference was used to interpret the data in FIGS. 9 and 10. 1=free fatty acids; 2=fatty alcohols, 3=phospholipids, 4=diacylglycerides; and 5=triacylglycerides.

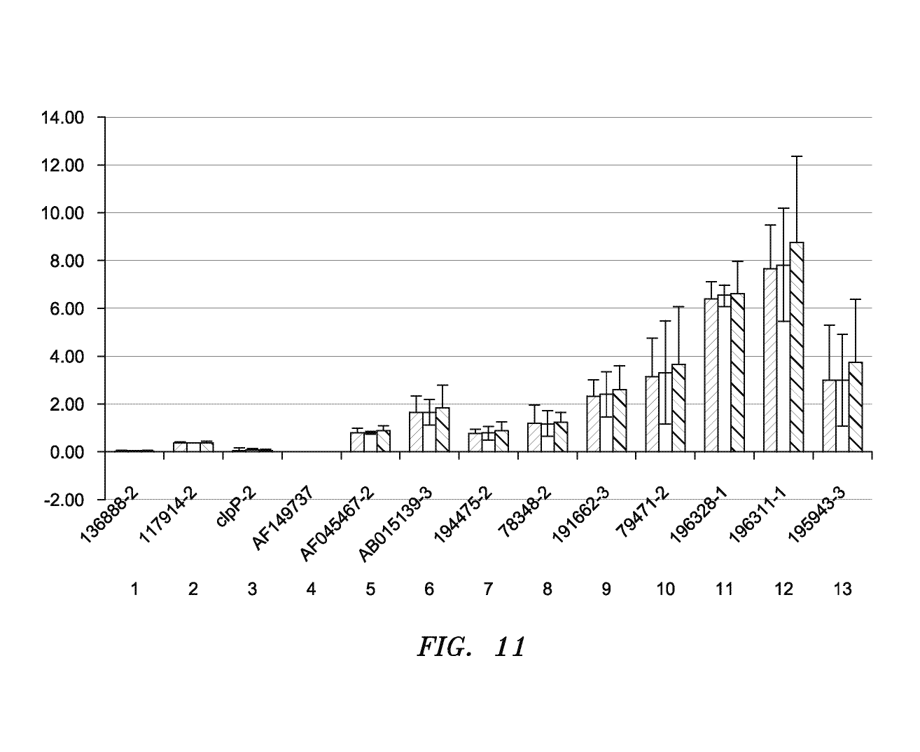

A range finding experiment was performed at the molecular level using qPCR on nitrogen replete and nitrogen starved samples (24 hour time point shown in FIG. 11). This experiment was conducted in order to find the molecular cues involved in the nitrogen starvation phenotypes. Target genes (listed along the X-axis and in Table 1) were selected based on expectations derived from the literature or pathways involved in nitrogen response. Wild-type Chlamydomonas reinhardtii cells were grown in 5-10 L of HSM media in a 5% carbon dioxide in an air environment under constant light, until cells reached early log phase. The culture was centrifuged at 3000 to 5000×g for 5-10 minutes and one half of the culture was washed with 500-1000 mL HSM, the other half with 500-1000 mL HSM containing no nitrogen. After re-centrifugation, the two cultures were resuspended in a volume of media (HSM or HSM containing no nitrogen) equivalent to the starting culture volume. At the time points listed in Table 2, 50-100 mL of the cells were harvested by centrifugation and RNA was purified from the cultures. 0.25-1.0 ug of RNA was combined with 0.25 ug human brain RNA (Biochain, Hayward, Calif.) as normalization control and used for iScript cDNA synthesis (BioRad, USA) and standard qPCR using iQ SybrGreen (BioRad, USA) detection. Significant upregulation (as shown by fold upregulation on the Y-axis) of 5 genes is seen within 24 hours of nitrogen starvation (as shown in FIG. 11). Triplicate qPCR reactions were run versus three human brain control genes (control gene in left hand column is PGAM1 (UniGene Hs.632918), middle column is BASP1 (UniGene Hs.201641), and right hand column is SLC25A14 (UniGene Hs.194686)).

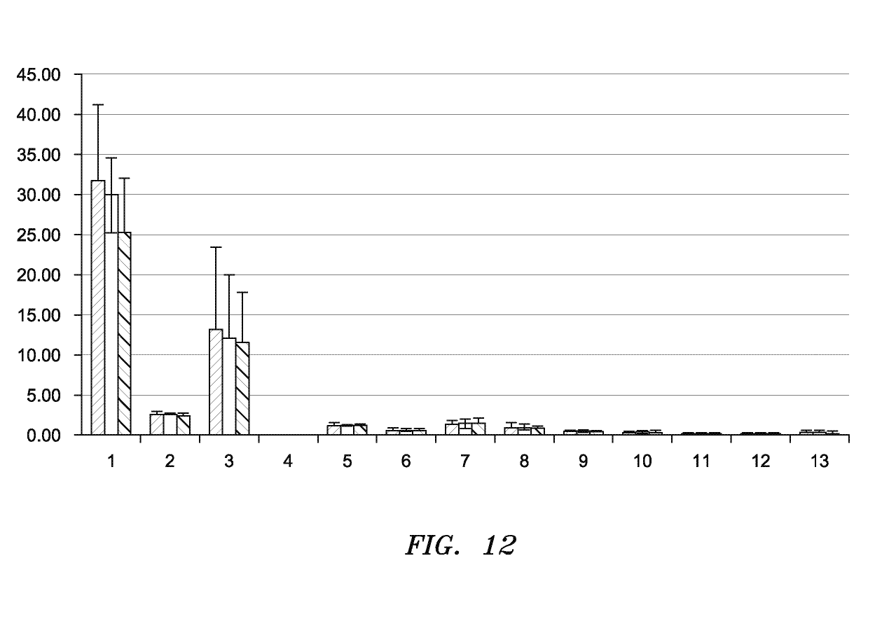

FIG. 12 shows gene expression changes (fold down regulation) in the same set of genes in Table 1 after 24 hours of nitrogen starvation. FIG. 12 contains the same data as FIG. 11, with FIG. 12 showing up regulation and FIG. 11 showing down regulation. Significant downregulation (as shown by fold downregulation on the Y-axis) of 3 genes is seen within 24 hours of nitrogen starvation. Similar changes (utip and down regulation) were also seen at the 6 hour time point. Triplicate qPCR reactions were run versus three control genes (control gene in left hand column is PGAM1 (UniGene Hs.632918), middle column is BASP1 (UniGene Hs.201641), and right hand column is SLC25A4 (UniGene Hs.194686)). These results indicate that molecular changes (as shown by qPCR in FIGS. 11 and 12) occur early and are seen prior to the lipid changes seen at 48 hours (as shown in FIGS. 9 and 10)

A key for the target genes used in the qPCR data shown in FIGS. 11 and 12 is provided below in Table 1. The below-listed genes are known Chlamydomonas reinhardtii genes. The first column indicates the fold up or down regulation at 24 hours. The second column indicates the fold up or down regulated at 48 hours. In the first and second columns, down regulation is indicated by (−) following the number and up regulation is indicated by (+) following the number.

These experiments show that the lipid accumulation and profile changes induced by nitrogen starvation begin primarily between 24 and 48 hours. The molecular changes (i.e. RNA expression) that are associated with nitrogen starvation begin earlier, with RNA expression level changes as early as 6 hours after nitrogen starvation.

TABLE 1

| 24 H | | 48 H | | # on x-axis | Gene |
|---|---|---|---|---|---|
| 29.0 | (−) | 19.1 | (−) | (1) 136888-2 | Glutamate synthase, NADH-dependent |
| | | | | (2) 117914-2 | Heat shock transcription factor 1 |
| 12.3 | (−) | 2.5 | (−) | (3) clpP-2 | L28803.1\|CRECLPP Chlamydomonas reinhardtii |
| | | | | | chloroplast Clp protease (clpP) gene |
| 4000 | (−) | 4000 | (−) | (4) AF149737 | Chlamydomonas reinhardtii |
| | | | | | nitrite transport NAR1 |
| | | | | (5) AF045467-2 | Chlamydomonas reinhardtii |
| | | | | | Ac115p (AC115) nuclear gene |
| | | | | | encoding chloroplast protein |
| 1.7 | (+) | 8.9 | (+) | (6) AB015139-3 | Chlamydomonas reinhardtii |
| | | | | | mRNA for chlorophyll a oxygenase |
| 0.8 | (+) | 25.0 | (+) | (7) 194475-2 | Porphobilinogen deaminase |
| | | | | (8) 78348-2 | beta subunit of mitochondrial ATP synthase |
| | | | | (9) 191662-3 | soluble starch synthase III |
| 3.4 | (+) | 2.6 | (+) | (10) 79471-2 | 2-oxoglutarate dehydrogenase, E1 subunit |
| 6.5 | (+) | 9.5 | (+) | (11) 196328-1 | malate synthase |
| 8.1 | (+) | 7.5 | (+) | (12) 196311-1 | Acetyl CoA synthetase |
| 3.3 | (+) | 5.9 | (+) | (13) 195943-3 | Uroporphyrinogen-III synthase |

### Example 3

### RNA-Sea Transcriptomic Method

In this example, an exemplary method used to identify the gene encoding SN03 is described. The method described herein can be used to identify other proteins, polypeptides, or transcription factors, for example, those involved in the regulation or control of different nitrogen deficient phenotypes found in an organism, for example, an alga. Such nitrogen deficient phenotypes include, for example, increased lipid production and/or accumulation, breakdown of photosystem, decreased growth, and mating induction. Genes identified as involved in regulation or control of different nitrogen deficient phenotypes could have positive or negative impacts on those phenotypes, for example, increased or decreased lipid production or increased or decreased growth rate.

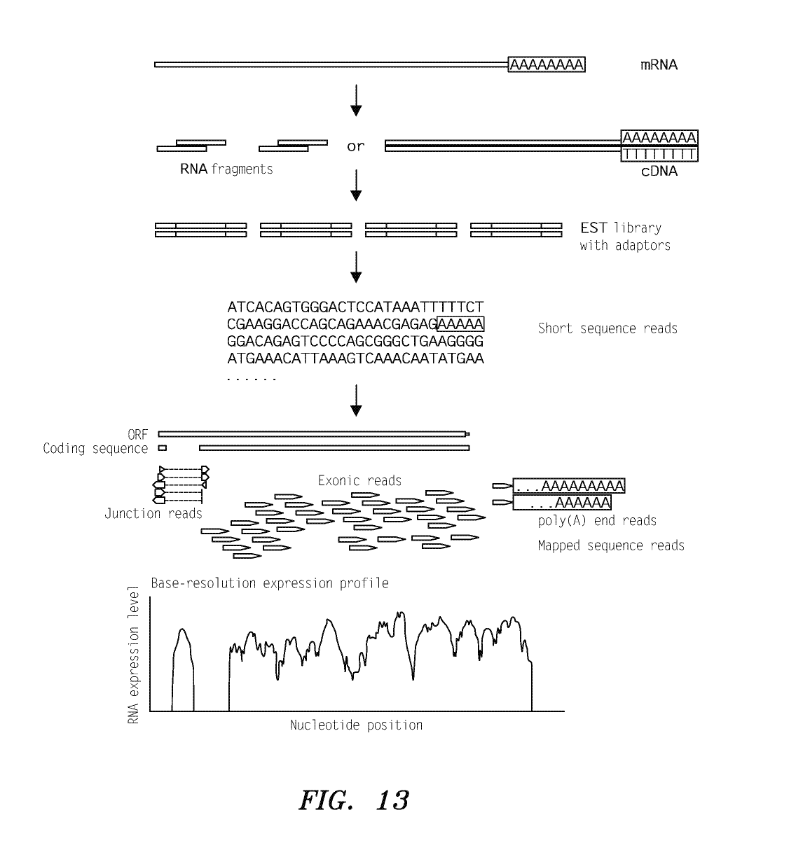

In order to identify genes/proteins involved in the nitrogen starvation induced lipid phenotype, the RNA-Seq transcriptomic method (FIG. 13; Wang, et al., Nat. Rev. Genet. (2009) vol. 10 (1) pp. 57-63) was used to determine expression levels of all genes in algae grown under six different conditions (listed in Table 2). These conditions were established based on the range finding experiments described in FIGS. 9, 10, 11 and 12. The RNA-Seq transcriptomic method is described below.

Briefly, mRNAs are first converted into a library of cDNA fragments through either RNA fragmentation or DNA fragmentation (see FIG. 13). Sequencing adaptors are subsequently added to each cDNA fragment (EST library with adapters) and a short sequence read is obtained from each cDNA fragment using high-throughput sequencing technology (Solexa). The resulting sequence reads are aligned with the reference transcriptome, and can be classified as three types: exonic reads, junction reads and poly(A) end-reads. These alignments are used to generate an expression profile for each gene, as illustrated at the bottom of FIG. 13; a yeast ORF with one intron is shown.

SE0050 RNA from six different conditions (exponential growth: +nitrogen; exponential growth: 6 hours−nitrogen; exponential growth: 24 hours−nitrogen; exponential growth: 48 hours−nitrogen; stationary phase: +nitrogen; and stationary phase: −nitrogen (approximately 11 days)) was prepared. Wild-type Chlamydomonas reinhardtii cells were grown in 5-10 L of HSM media in a 5% carbon dioxide in an air environment under constant light, until cells reached early log phase. The culture was centrifuged at 3000 to 5000×g for 5-10 minutes and one half of the culture was washed with 500-1000 mL HSM, the other half with 500-1000 mL HSM containing no nitrogen. After re-centrifugation, the two cultures were resuspended in a volume of media (HSM or HSM containing no nitrogen) equivalent to the starting culture volume. At the time points listed in Table 2, 50-100 mL of the cells were harvested by centrifugation and RNA was purified from the cultures. This RNA was sequenced using standard Solexa methodologies (Sequensys, Inc, La Jolla, Calif.) for use in the RNA-Seq analysis method. Between 3.8 million to 17.8 million 36-mer reads were generated per sample (see Table 2).

This RNA-Seq transcriptomic data was mapped against version 3.0 of the Department of Energy (DOE) Joint Genome Institute's (JGI) Chlamydomonas reinhardtii genome using Arraystar software (DNASTAR, USA). The set of genes used for the mapping included 16,824 annotated nuclear genes. JGI's functional annotations (version 3.0) were also used and imported into the Arraystar software. Most of these annotations are based on prediction algorithms and do not have supporting experimental evidence. A small fraction have supporting experimental evidence. Approximately 7,500 have functional annotations of some kind. The JGI functional annotations used included KOG (clusters of orthologous genes), EC (Enzyme Commission numeric assignments), and GO (Gene Ontology).

SE0050 Solexa data mapped to version 3.0 transcripts. 4-18 million reads were generated for each sample and mapped to the genome, representing over 2GBases of data—2 billion+ nucleotides. Presented below in Table 2 are the total number of Solexa 36 bp reads generated for each of the six RNA samples. Also shown for each sample are the number of those reads that successfully mapped to the Chlamydomonas reinhardtii v3.0 transcriptome (total reads with mer hits) and the percentage of total hits mapped to the transcriptome.

TABLE 2

Exp +N
Total Sample reads: 10,071,444
Total reads with mer hits: 6,468,875
Percentage mapped: 64.2
Stationary +N
Total Sample reads: 3,871,450
Total reads with mer hits: 2,523,731
Percentage mapped: 65.2
6 H −N
Total Sample reads: 7,606,940
Total reads with mer hits: 4,965,650
Percentage mapped: 65.3
24 H −N
Total Sample reads: 7,709,562
Total reads with mer hits: 5,021,348
Percentage mapped: 65.1
48 H −N
Total Sample reads: 10,644,517
Total reads with mer hits: 6,691,219
Percentage mapped: 62.9
Stationary −N
Total Sample reads: 17,799,413
Total reads with mer hits: 8,761,230
Percentage mapped: 49.2

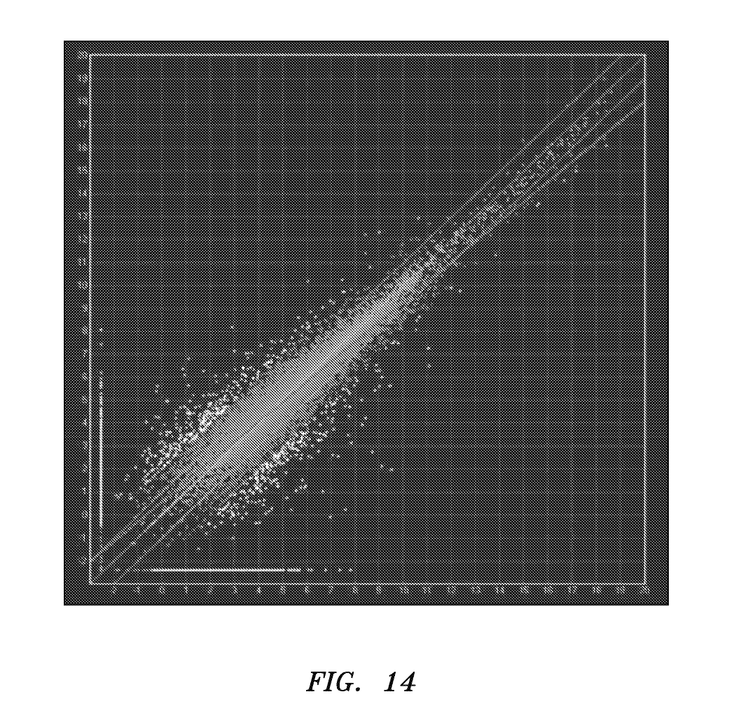

The transcriptomic data was then analyzed by looking at changes in expression levels between the six samples and across the time course of nitrogen starvation. FIG. 14 shows a plot of all 16,000+ genes in SE0050 with expression levels from a different sample on each axis. Shown here are Exponential growth +Nitrogen (x-axis) versus Exponential growth 6 H −Nitrogen (y-axis). Genes with no change in expression level are on the diagonal. The white data points represent at least 4-fold change in expression, those above the diagonal are upregulated after 6 hours of nitrogen starvation and those below the diagonal are down regulated after 6 hours of nitrogen starvation. These plots can be generated for any pair wise comparison of the six sequenced samples. These expression profiles were used in selecting target genes.

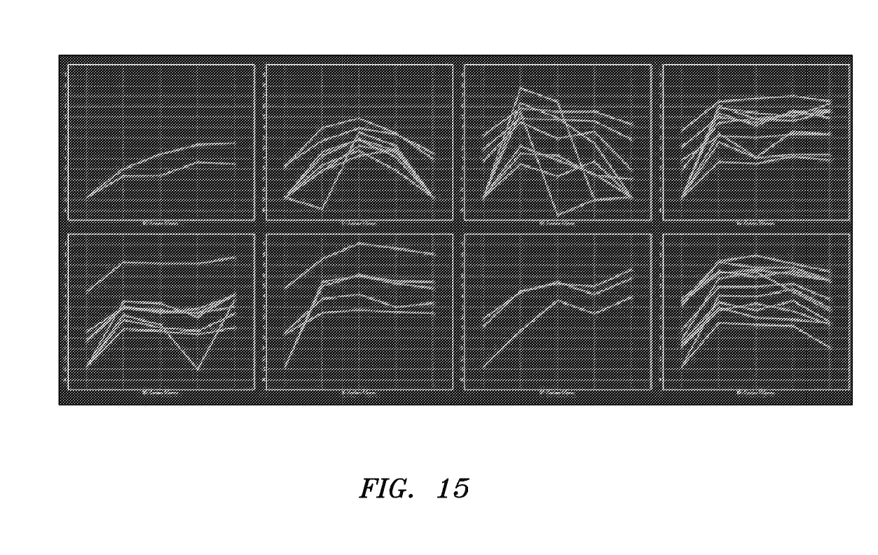

Example of time course of expression (as mentioned above regarding FIG. 14). FIG. 15 shows how the dynamics of gene expression during nitrogen starvation (6 H, 24 H, 48 H, stationary) were used to further refine the target gene list. Each line represents one gene, with the y axis in each case being the level of expression and the x axis representing the 6 samples sequenced. The eight graphs represent genes that have similar expression patterns across the conditions represented by the 6 samples. These patterns and groupings can be used to further refine target gene lists.

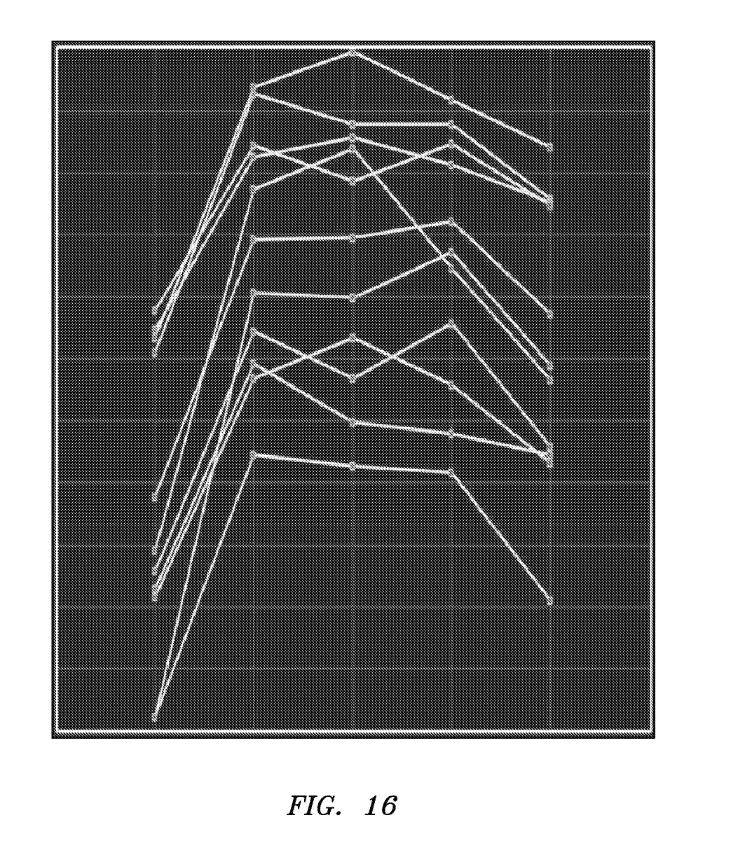

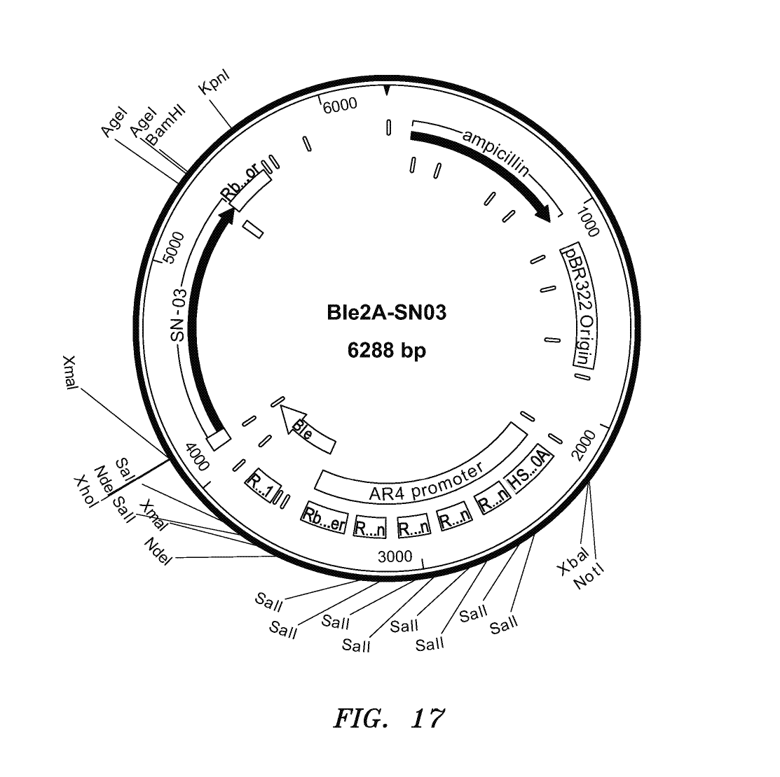

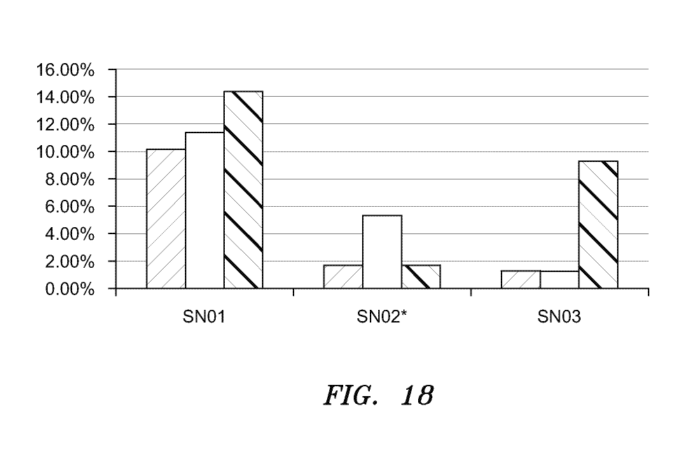

FIG. 16 shows the expression pattern for 14 genes that had expression patterns indicating that the genes were turned on quickly after nitrogen starvation and stayed on. The 14 genes represent the lower right hand box of FIG. 15. This set of 14 was selected because the functional annotations from JGI indicated that these genes were expected to be involved in transcription and/or gene regulation. Genes that potentially control the nitrogen starvation response and are expected to be regulatory genes were selected as targets. The completeness of the JGI gene annotation at the molecular level also determines the usability of potential targets. For example, many of the annotated genes do not have start and/or stop codons, and therefore the complete open reading frame (ORF) is unknown. The initial 14 targets were limited to 5 due to poor annotation. 3 of the 14 did not have start codons, 3 did not have stop codons, 2 had neither start nor stop codons, and 1 had an inappropriate stop codon. The five selected targets were full length ORFs with start and stop codons.

### Example 4

### Cloning of SN03 into Ble2A

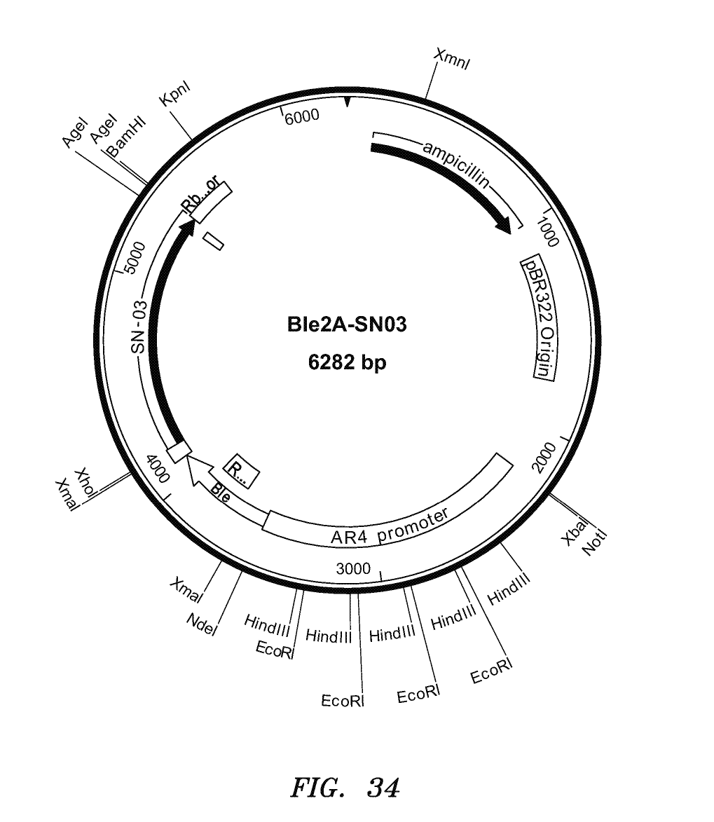

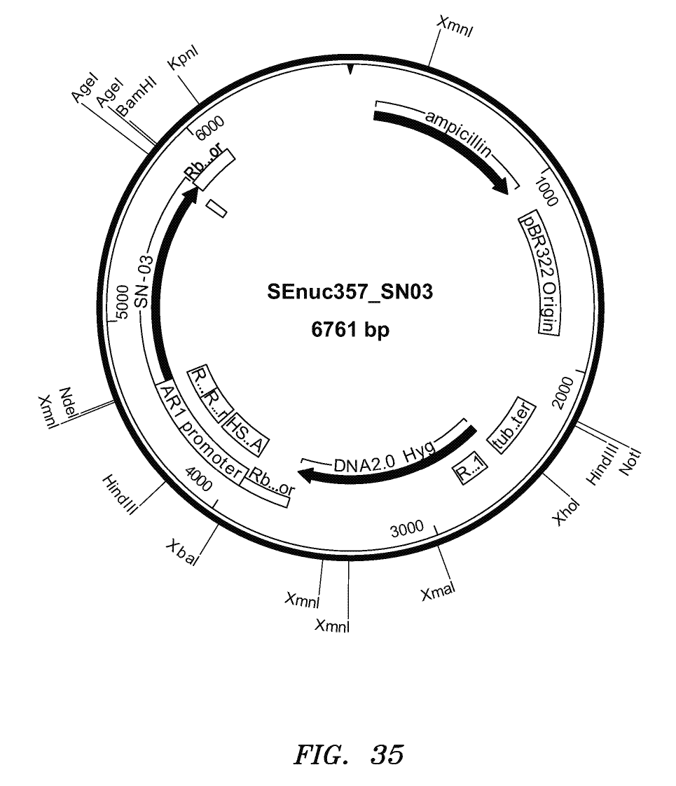

The ORFs for SN03 was codon optimized for the nuclear genome of Chlamydomonas reinhardtii using Chlamydomonas reinhardtii codon usage tables, and synthesized. The DNA constructs for SN03 was cloned into nuclear overexpression vector Ble2A (as shown in FIG. 34) and transformed into SE0050. This construct produces one RNA with a nucleotide sequence encoding a selection protein (Ble) and a nucleotide sequence encoding a protein of interest. The expression of the two proteins are linked by the viral peptide 2A (for example, as described in Donnelly et al., J Gen Virol (2001) vol. 82 (Pt 5) pp. 1013-25). This protein sequence facilitates expression of two polypeptides from a single mRNA.

TABLE 3

| | |
|---|---|
| SN03 | CREB binding protein/P300 and related TAZ Zn-finger |
| | proteins JGI Chlre v3 protein ID # 147817 |

Transforming DNA, the Ble2A-SN03 plasmid shown in FIG. 34, was created by using pBluescript II SK(−) (Agilent Technologies, CA) as a vector backbone. The segment labeled “AR4 Promoter” indicates a fused promoter region beginning with the C. reinhardtii Hsp70A promoter, C. reinhardtii rbcS2 promoter, and the four copies of the first intron from the C. reinhardtii rbcS2 gene (Sizova et al. Gene. 277:221-229 (2001)). The gene encoding bleomycin binding protein was fused to the 2A region of foot-and-mouth disease virus and the SN ORF with a FLAG-MAT tag cloned in with XhoI and BamHI. This was followed by the Chlamydomonas reinhardtii rbcS2 terminator.

Transformation DNA was prepared by digesting the Ble2A-SN vector with the restriction enzyme KpnI, XbaI or PsiI followed by heat inactivation of the enzyme. For these experiments, all transformations were carried out on C. reinhardtii cc1690 (mt+). Cells were grown and transformed via electroporation. Cells were grown to mid-log phase (approximately 2-6×10⁶ cells/ml) in TAP media. Cells were spun down at between 2000×g and 5000×g for 5 min. The supernatant was removed and the cells were resuspended in TAP media+40 mM sucrose. 250-1000 ng (in 1-5 μL H₂O) of transformation DNA was mixed with 250 μL of 3×10⁸ cells/mL on ice and transferred to 0.4 cm electroporation cuvettes. Electroporation was performed with the capacitance set at 25 uF, the voltage at 800 V to deliver 2000 V/cm resulting in a time constant of approximately 10-14 ms. Following electroporation, the cuvette was returned to room temperature for 5-20 min. For each transformation, cells were transferred to 10 ml of TAP media+40 mM sucrose and allowed to recover at room temperature for 12-16 hours with continuous shaking. Cells were then harvested by centrifugation at between 2000×g and 5000×g, the supernatant was discarded, and the pellet was resuspended in 0.5 ml TAP media+40 mM sucrose. The resuspended cells were then plated on solid TAP media+20 μg/mL zeocin. As a result, overexpression lines for SN03 were created.

### Example 5

### Lipid Dye/Flow Cytometry Analysis on SN03

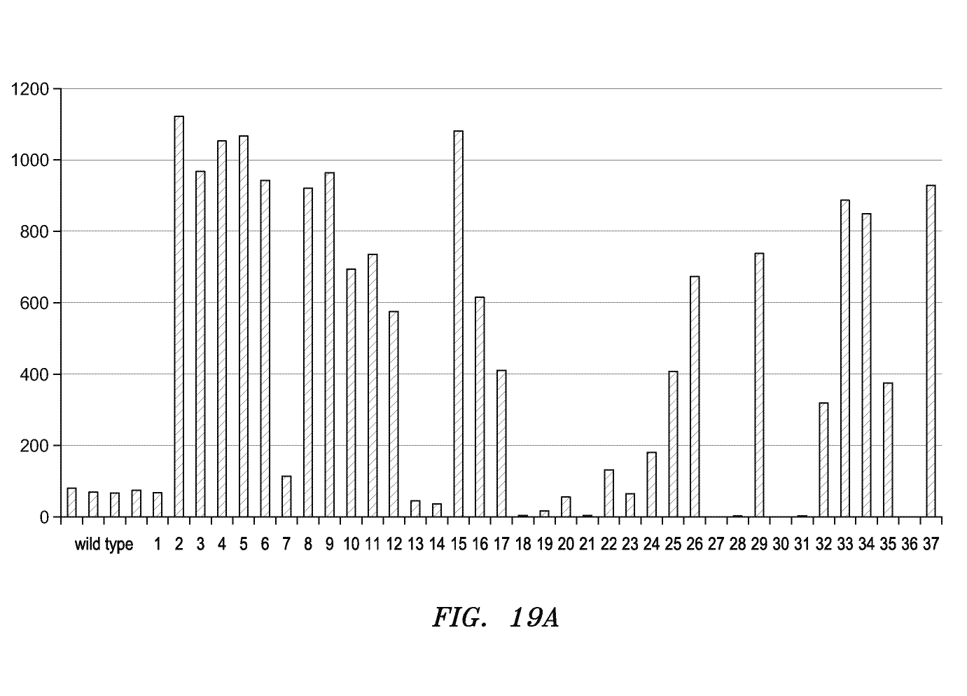

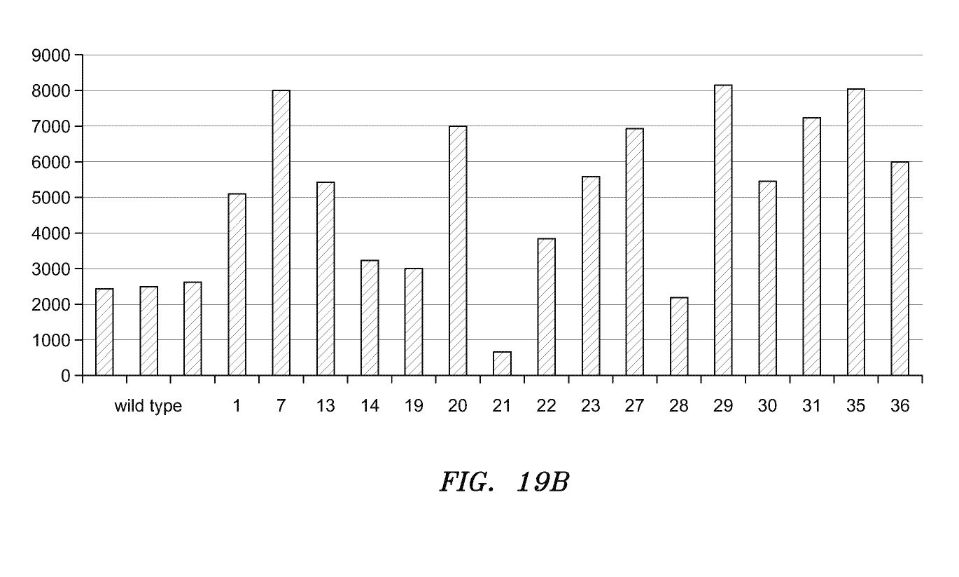

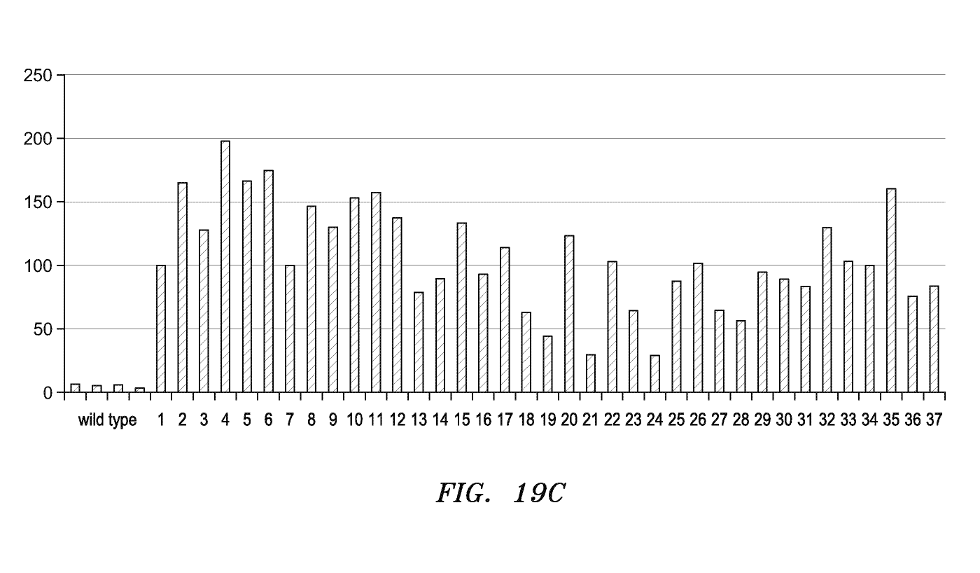

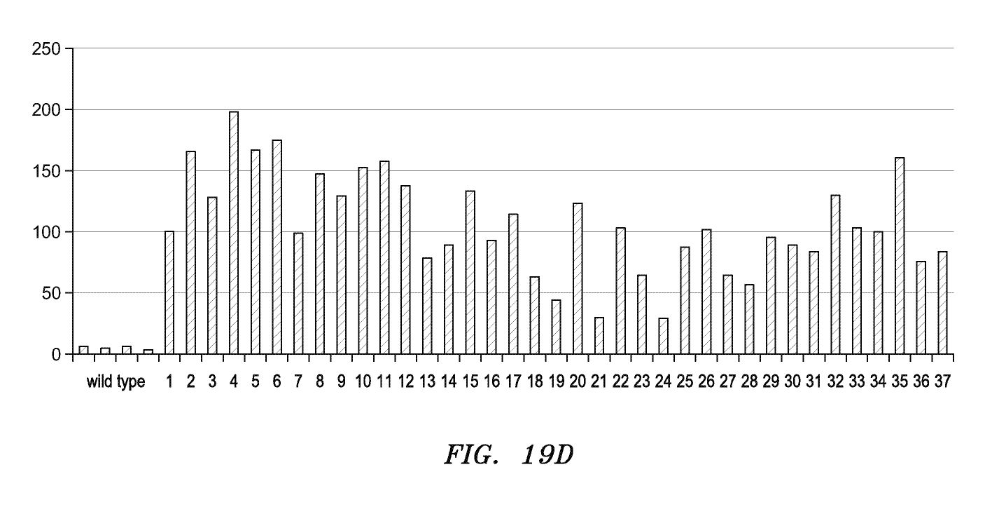

37 individual SN03 colonies were screened by flow cytometry (Guava) using three lipid dyes. Cells were grown in 1-5 mL of TAP to mid-log phase, then diluted into media containing the lipid dyes before analysis on the flow cytometer (Guava). Overall, the SN03 lines show higher lipid dye staining than wild type (wt 1-4 are biological replicates of wild type), again suggesting that they have more lipid. FIG. 19A shows Bodipy staining. FIG. 19B shows a repeated Bodipy staining; FIG. 19C shows LipidTOX staining; and FIG. 19D shows Nile Red staining. The x-axis represents individual strains, whether wild type or the 37 SN03 overexpressing lines (named SN03-1 to SN03-37) while the y-axis represents relative fluorescence units.

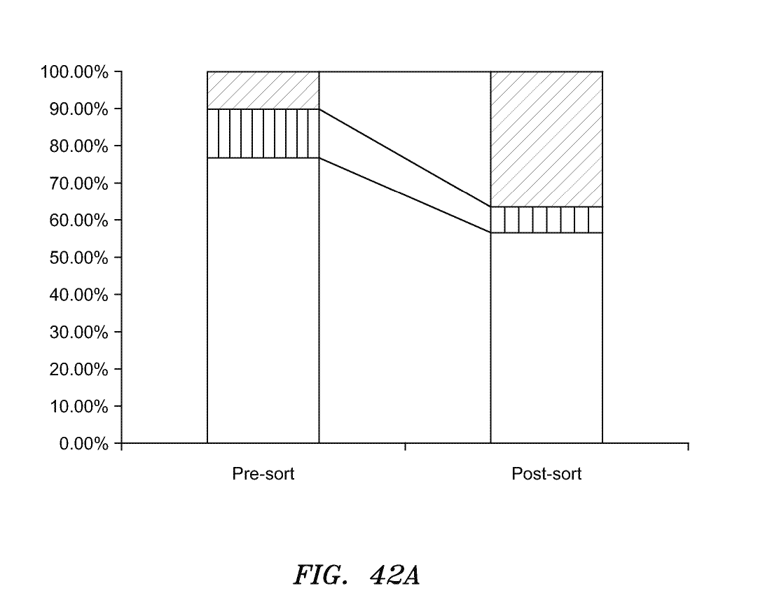

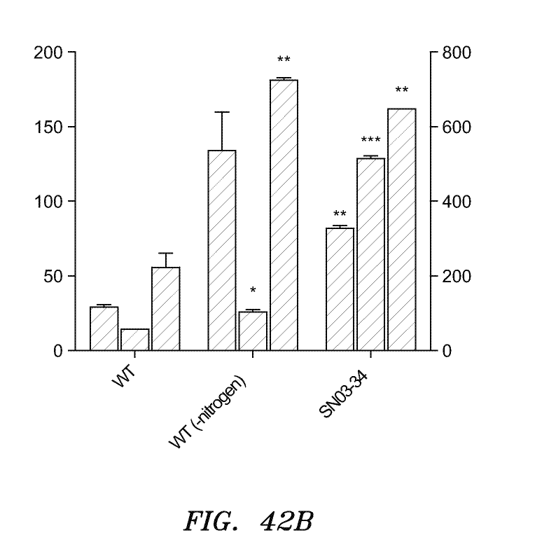

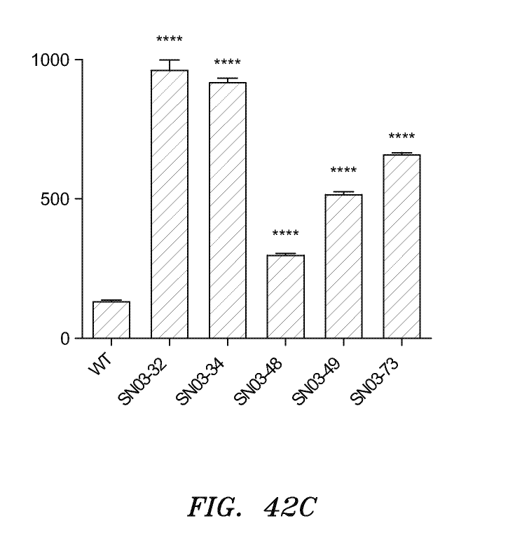

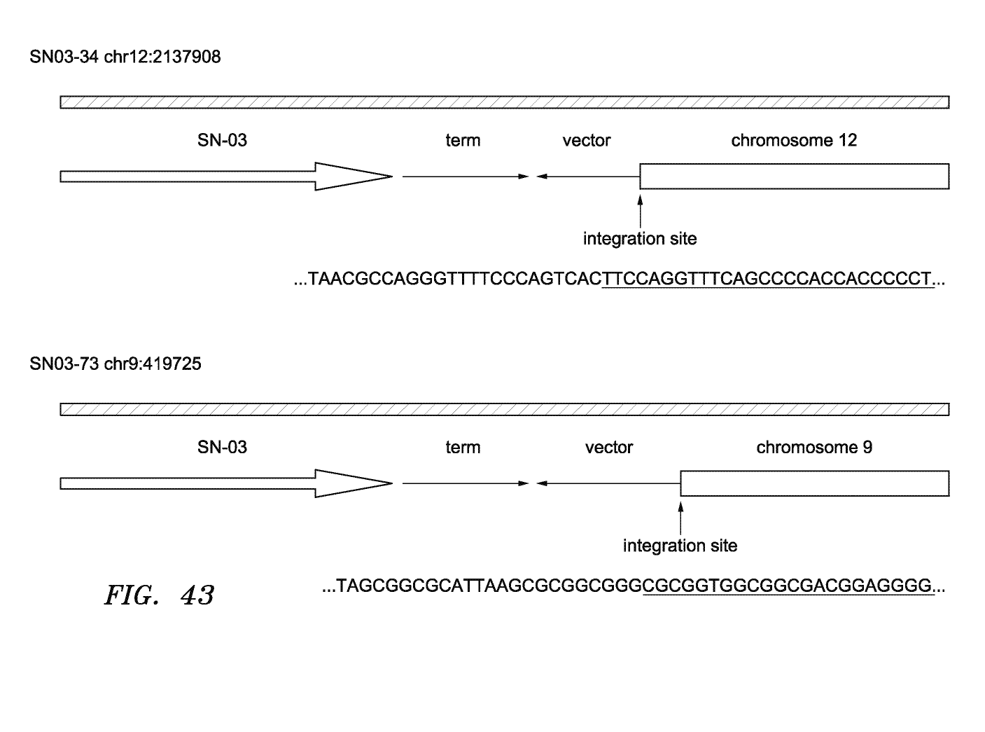

FIG. 42B shows the lipid content as determined by lipid dyes and flow cytometry (Guava) in wild type Chlamydomonas reinhardtii grown in the presence and absence of nitrogen and an SN03 overexpression line. Wild-type Chlamydomonas reinhardtii cells were grown in 10-100 mL of TAP media containing 7.5 mM NH4Cl in an air environment under constant light, until cells reached early log phase. The culture was centrifuged at 3000 to 5000×g for 5-10 minutes and one half of the culture was washed with 5-100 mL TAP, the other half with 5-100 mL TAP containing no nitrogen. After re-centrifugation, the two cultures were resuspended in a volume equivalent to the starting culture volume. Additionally, one SN03 overexpression line was grown in 10-100 mL of TAP media containing 7.5 mM NH4Cl in an air environment under constant light, until cells reached early log phase. After 2-3 days of nitrogen starvation for the wild type culture, the cultures were diluted into media containing lipid dye before analysis on the flow cytometer (Guava). Three dyes were used independently. In FIG. 42B, the x axis indicates the sample for each set of three dyes represented by the columns. In each set of three columns, the left column represents Nile Red, the middle column represents LipidTOX Green and the right column represents Bodipy. The left y axis shows relative fluorescence units (RFU) for Nile Red and LipidTOX Green (NR, LT), while the right y axis shows RFU for Bodipy. The SN03 overexpression line shows lipid staining higher than wild type in the presence of nitrogen and comparable to wild type in the absence of nitrogen.

FIG. 42C shows the lipid content of several independent SN03 overexpression lines. Wild type Chlamydomonas reinhardtii and five SN03 overexpression line were grown in 10-100 mL of TAP media containing 7.5 mM NH4Cl in an air environment under constant light, until cells reached early log phase. The cultures were diluted into media containing Bodipy before analysis on the flow cytometer (Guava). The x axis indicates wild type (wt) or the SN03 overexpression line, while the y axis indicates relative fluorescence units (RFU). All five SN03 overexpression lines show lipid staining higher than wild type.

### Example 6

### Phenotypic Analysis of SN03 Overexpression Lines

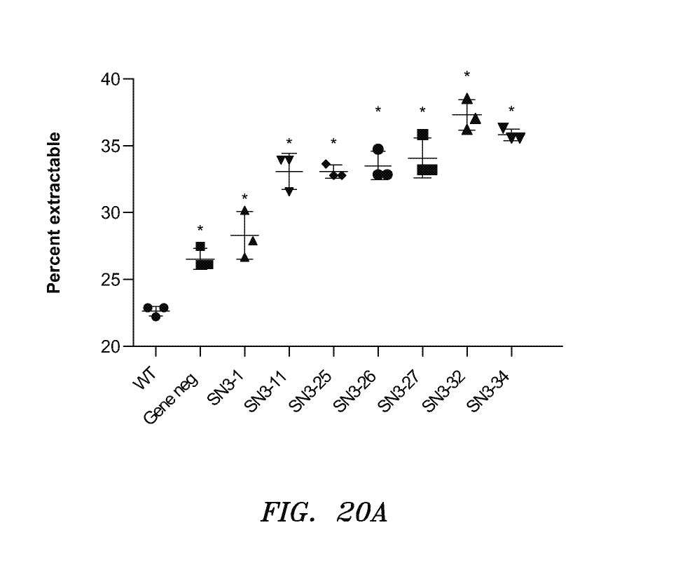

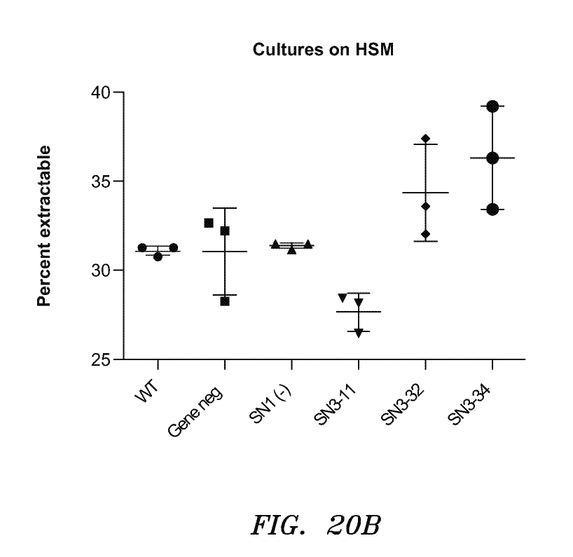

Seven of the SN03 transgenic lines along with the wild-type cells (FIG. 20A) were grown in TAP media in an air environment under constant light, until cells reached late log phase. Separately, three of the SN03 transgenic lines along with a transgenic line that does not contain an SN gene (gene neg), one SN01 transgenic line and wild type (FIG. 20B) were grown in HSM media in a 5% carbon dioxide in an air environment under constant light, until cells reached late log phase. 1-2 L of cells were harvested by centrifugation and analyzed for total gravimetric lipids by methanol/methyl-tert-butyl ether extraction according to a modified Bligh Dyer method (as described in Matyash V., et al. (2008) Journal of Lipid Research 49:1137-1146).

Specifically, biomass was pelleted and excess water removed. After the addition of methanol, samples were vortexed vigorously to lyse cells. MTBE was added and samples were vortexed again for an extended period of time (approximately 1 hr). Addition of water to samples after vortexing gave a ratio of 4:1.2:1; MTBE:MeOH:water respectively. Samples were centrifuged to aid in phase separation. The organic layer was removed and the process repeated a second time. Samples were extracted a third time adding only MTBE; the samples were vortexed, centrifuged, and phase separated as described above. The organic layers were combined, dried with magnesium sulfate, filtered and concentrated into tared vials. The percent extractables was calculated using the ash free dry weight of the sample.

FIGS. 20A and B show data points with error bars at mean+/−standard deviation. The y-axis represents percent extractables and the x-axis represents the strains as described above. The samples were different at p<0.05 from wild type marked with star. SN03 lines have significantly more lipid than the wild type line.

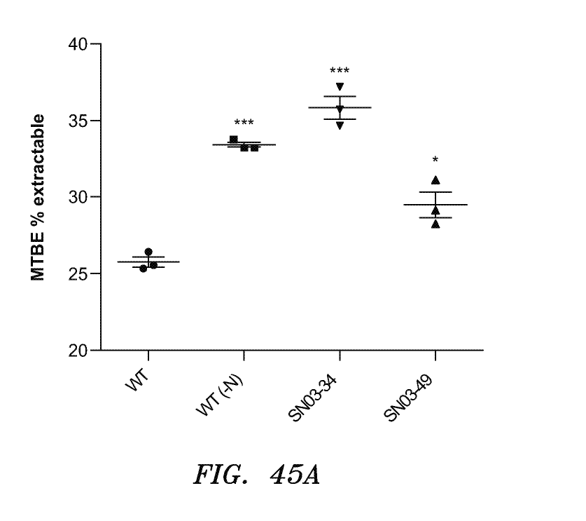

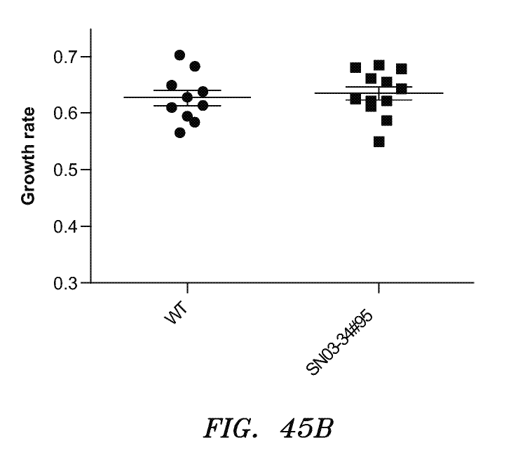

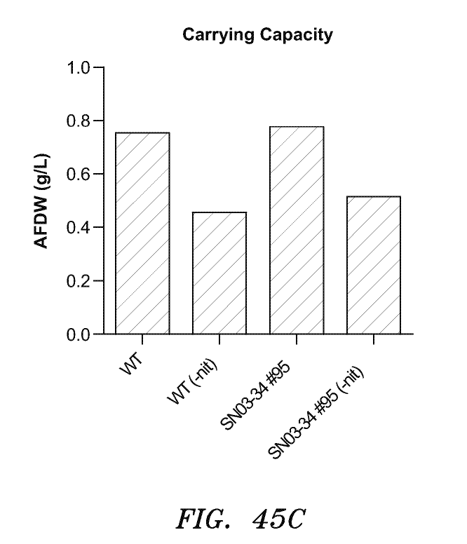

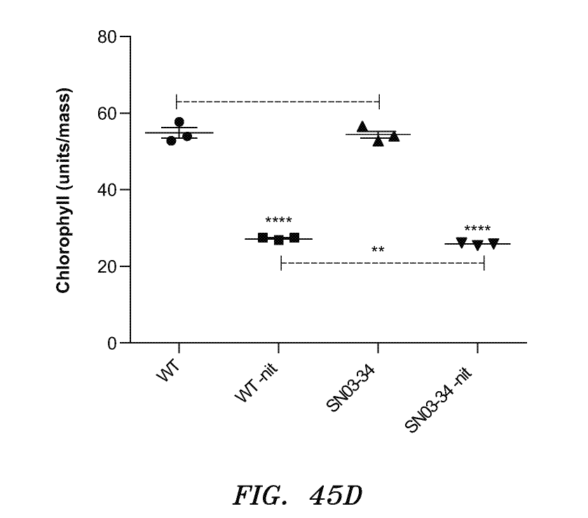

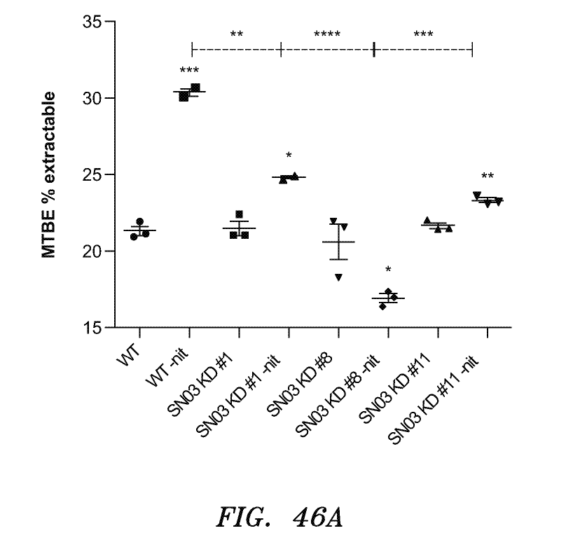

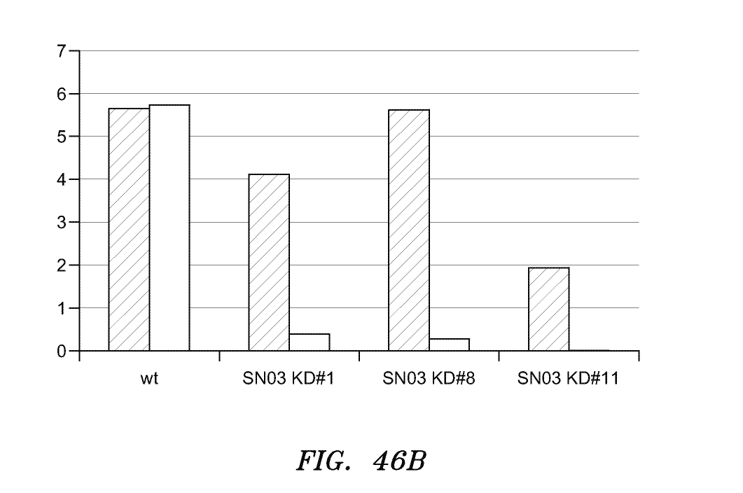

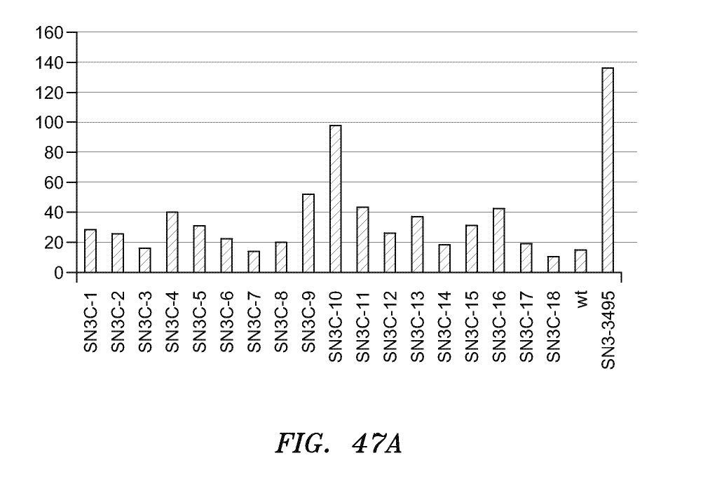

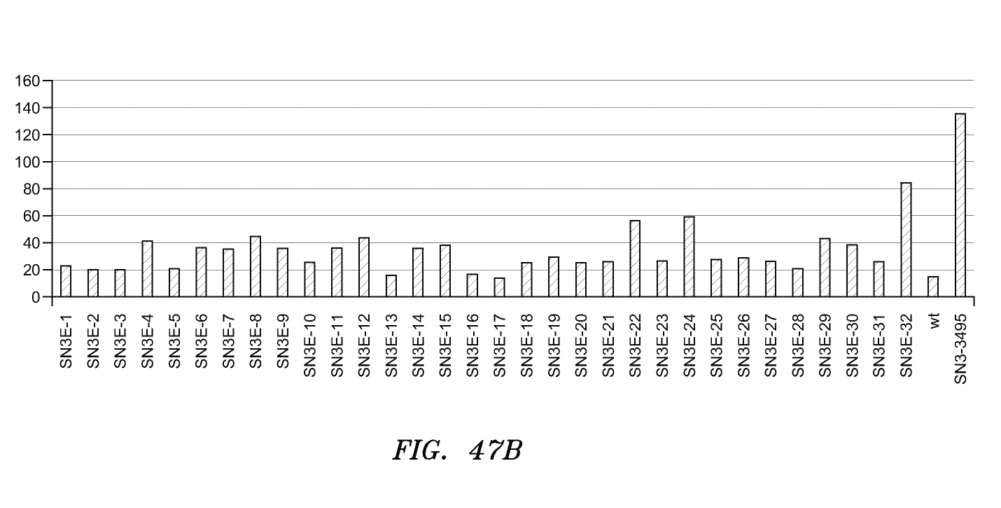

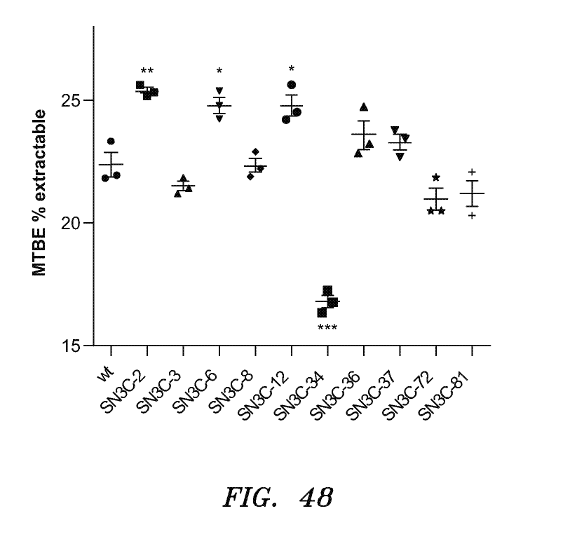

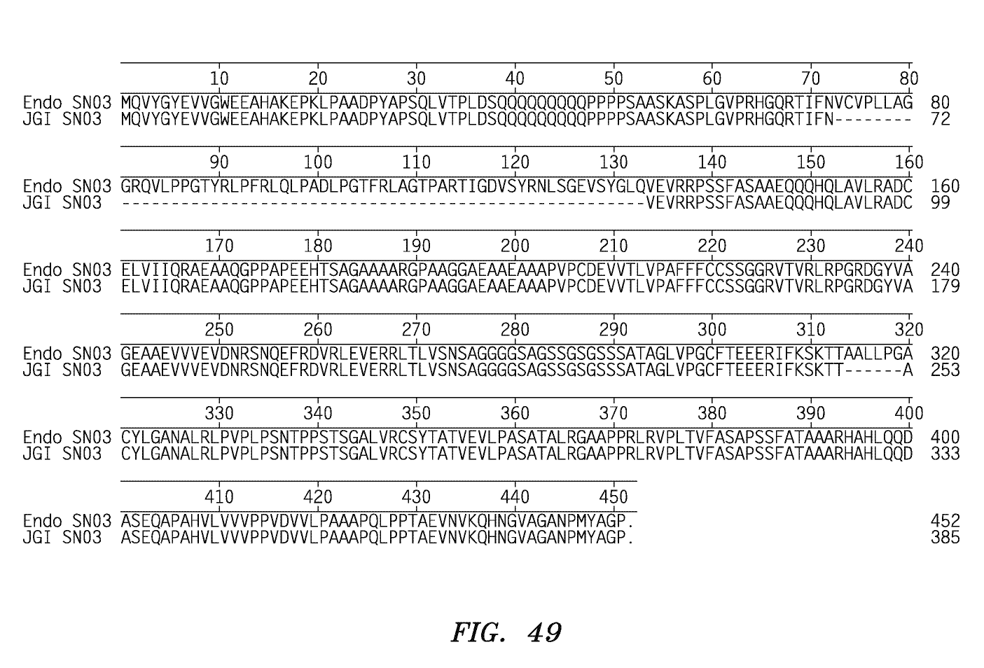

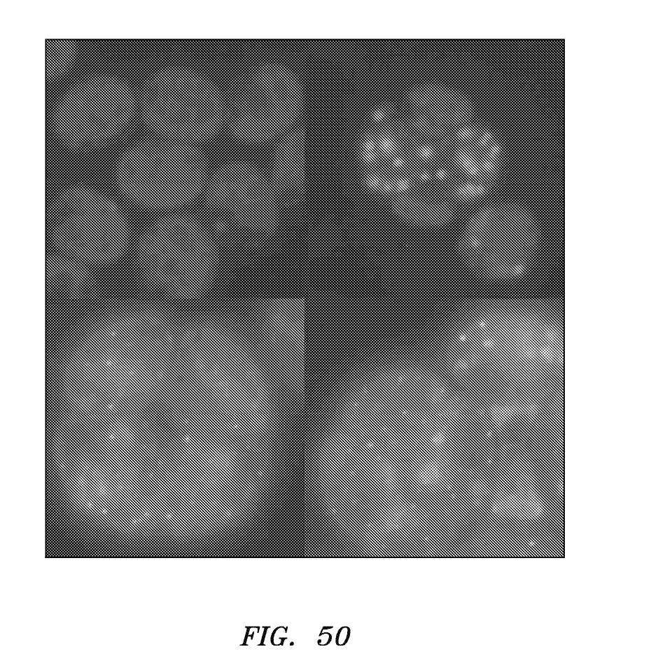

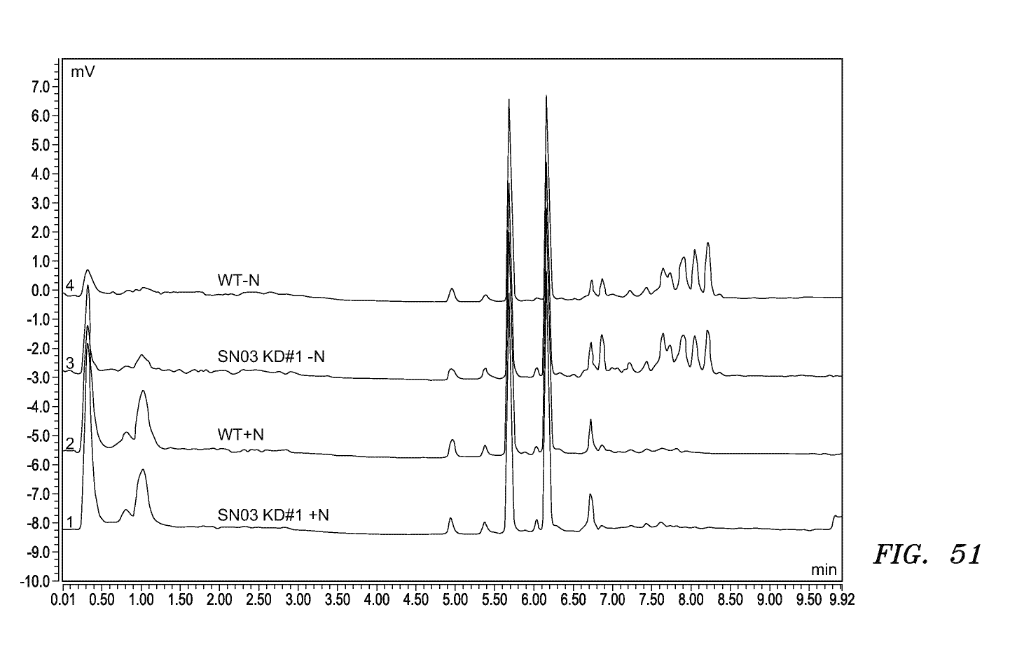

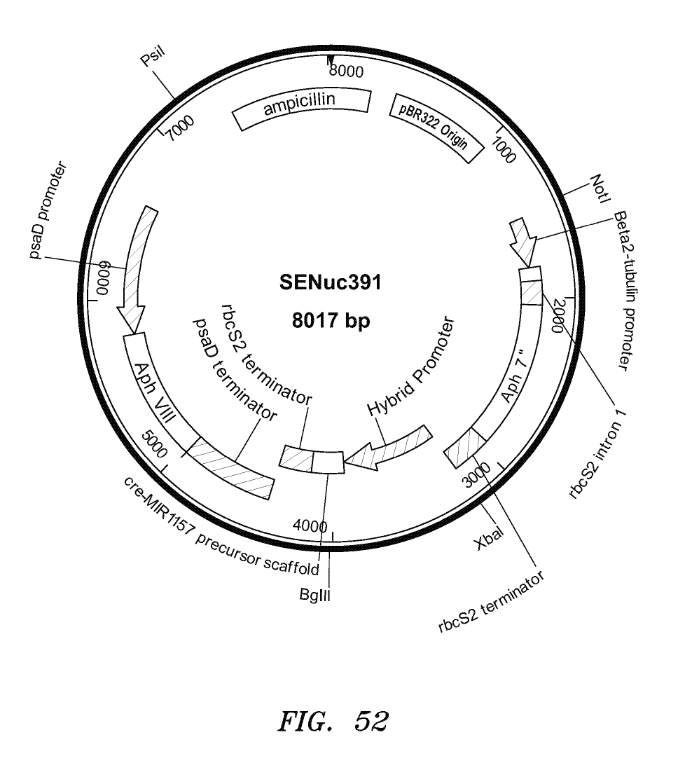

FIG. 45A is an additional example showing that SN03 overexpression lines accumulate more lipids than wild type. Wild-type Chlamydomonas reinhardtii cells were grown in 1-2 L of TAP media containing 7.5 mM NH4Cl in an air environment under constant light, until cells reached early log phase. The culture was centrifuged at 3000 to 5000×g for 5-10 minutes and one half of the culture was washed with 100-500 mL TAP, the other half with 100-500 mL TAP containing no nitrogen. After re-centrifugation, the two cultures were resuspended in a volume equivalent to the starting culture volume. Additionally, two SN03 overexpression lines were grown in 1-2 L of TAP media containing 7.5 mM NH4Cl in an air environment under constant light, until cells reached early log phase. After 2-3 days of nitrogen starvation for the wild type culture, cells were harvested by centrifugation and analyzed for total gravimetric lipids by methanol/methyl-tert-butyl ether extraction according to a modified Bligh Dyer method (as described in Matyash V., et al. (2008) Journal of Lipid Research 49:1137-1146). FIG. 45A shows data points with error bars at mean+/−standard deviation. The y-axis represents percent extractables and the x-axis represents the strains as described above. The samples were different at p<0.05 from wild type marked with star. SN03 lines have significantly more lipid than the wild type line and levels comparable to wild type in the absence of nitrogen.

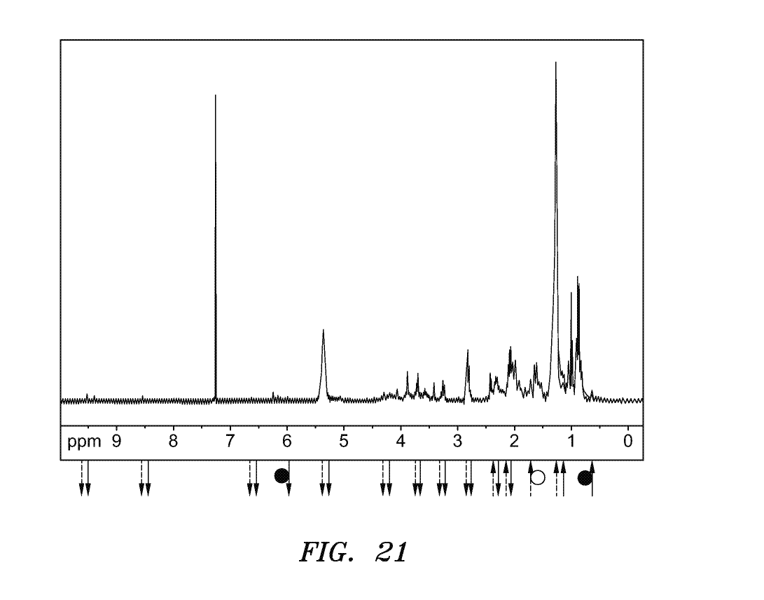

FIG. 21 is a comparison of 1-D 1H NMR spectra of MTBE:MeOH extracts (wild-type, SN3 gene positive, and nitrogen starved) taken from the samples described in FIG. 20a. Samples were dissolved in CDCl₃ prior to collection of NMR spectra.

Comparison of 1D proton NMR spectra of MTBE:methanol extracts of nitrogen replete wild type. SN3-34, and nitrogen starved wild type cultures. Peaks with differences in relative integrals marked with arrows. Direction of change of integral area from nitrogen replete wild type to SN3-34 is shown by the left arrow for each peak. Direction of change of integral area from nitrogen replete wild type to nitrogen starved wild type is shown by the right arrow for each peak. For most peaks, the direction of change in peak area (relative increase or decrease in component concentration) is the same for wild type undergoing nitrogen stress and SN3-34 overexpression.

These figures show that the SN03 lipid profile is similar to the profile of oil from nitrogen starved cultures, while both are different as compared to oil from wild type cultures. This shows that the nitrogen stress response has been turned on by over expressing SN03.

For most peaks, the direction of change in peak area is the same for cells expressing SN3 or for cells undergoing nitrogen stress.

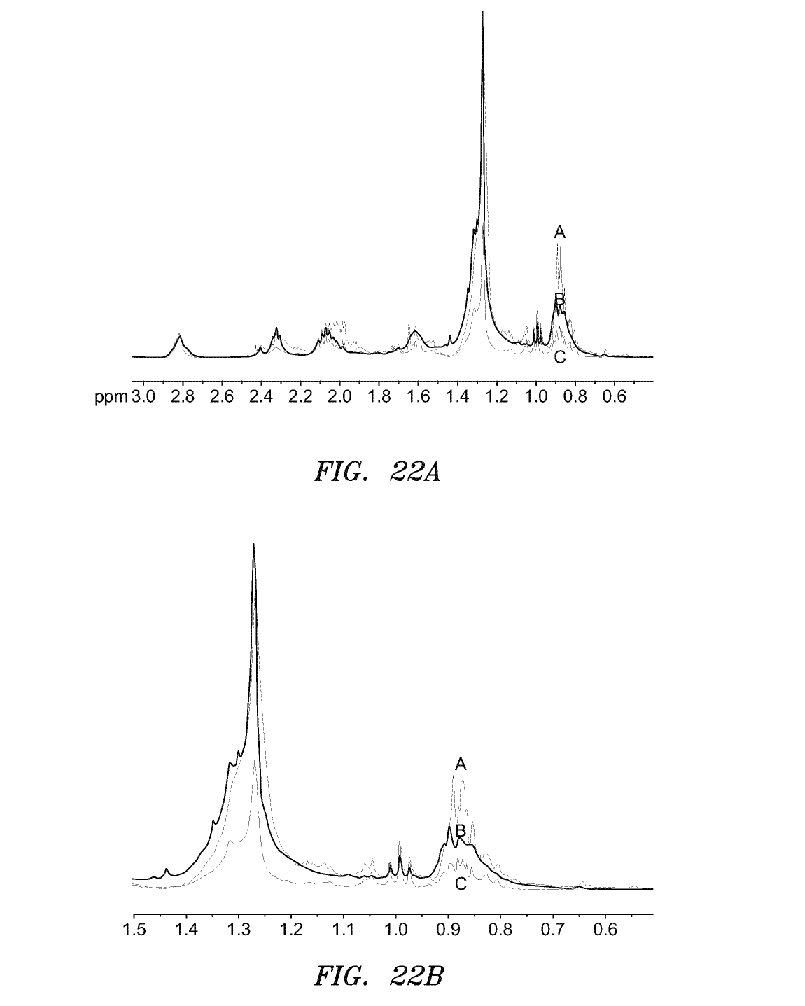

FIGS. 22A and B are close ups of the NMR peaks from FIG. 21. The SN03 and starved oil samples are similar and both are distinct from wild type oil. Again the SN03 lines mimic the stress response. Saturated methylene peaks appear at 1.27 ppm and terminal methyl peaks appear at 0.88 ppm. Starved wild type and SN03-34 spectra are similar to each other (relative to unstarved wild type). Normalized to peak at 2.8 ppm, wild type starved (B), wild type replete (C), and SN 3-34 replete (A). Comparison of nitrogen replete wild type, nitrogen starved wild-type, and SN03-34 MTBE:Methanol extract proton NMR spectra in CDCl₃. The SN3-34 spectrum (A) and wild-type starved (B) are similar at most peak positions, while wild-type replete (C) is different.

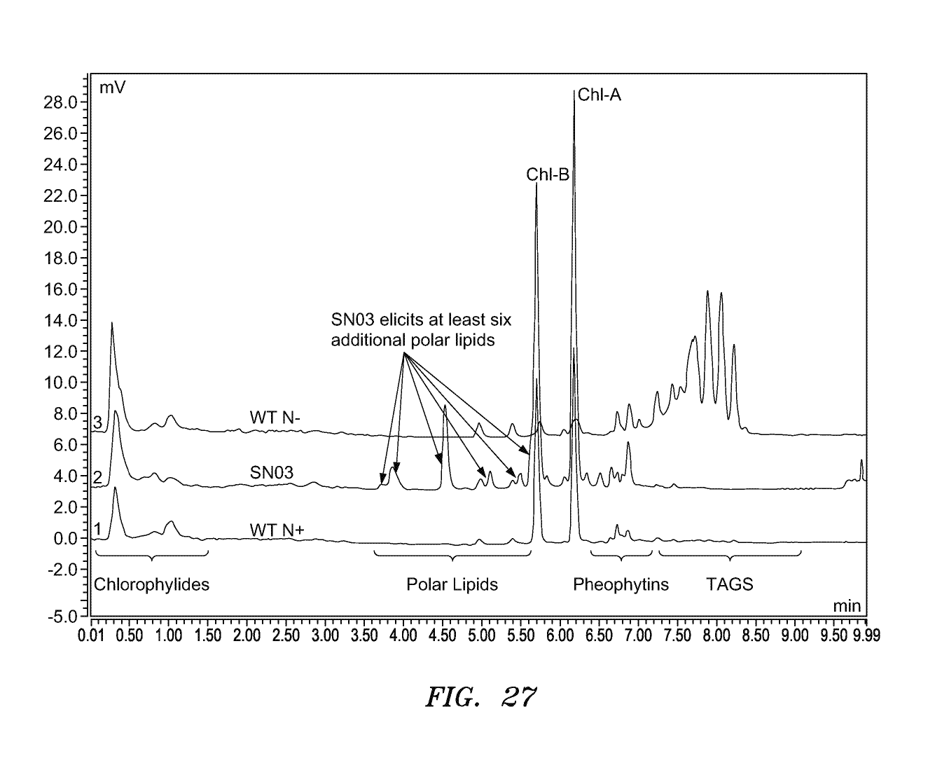

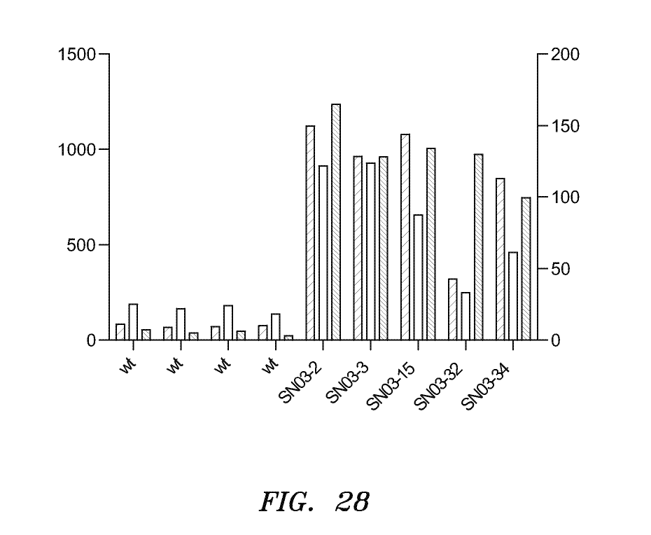

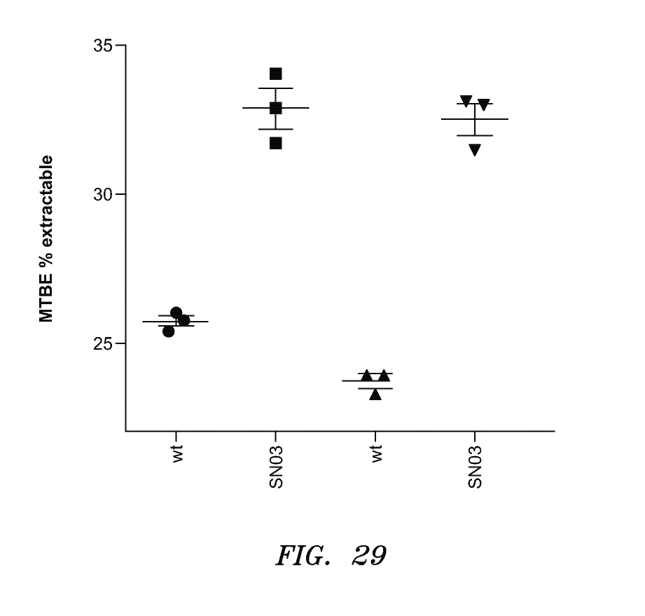

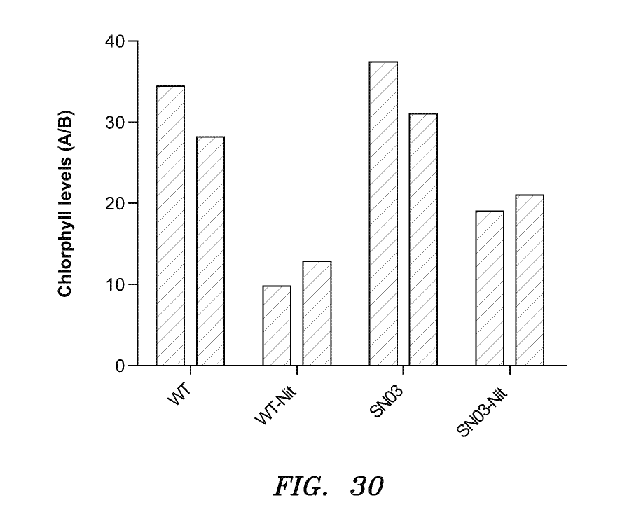

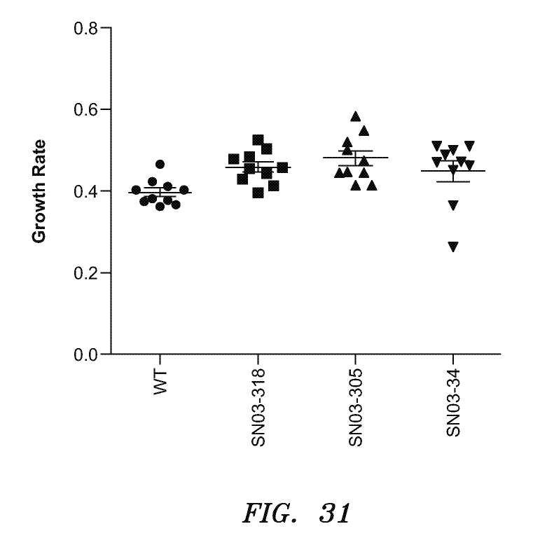

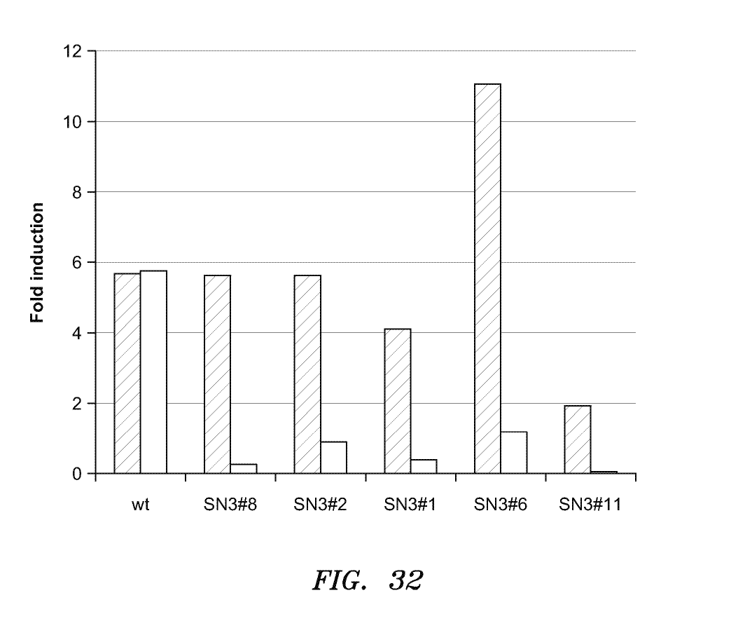

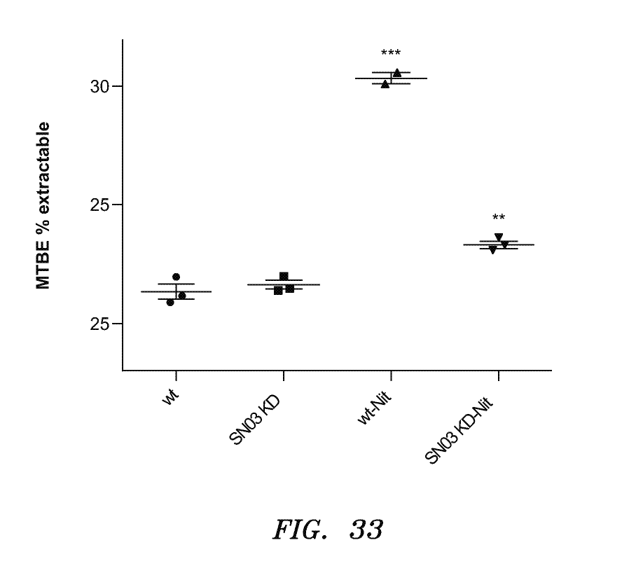

FIG. 27 is HPLC data showing the differences seen between MTBE extracted oil from an SN03 overexpression line and from Chlamydomonas reinhardtii wild type grown in the presence or absence of nitrogen. MTBE extracted oils were run on reverse-phase HPLC on a C18 column. Mobile phase was Acetonitrile/water/THF run over 10 minutes and flow rate of 0.9 mL/min. Detection was via an Evaporative Light Scattering Detector (ELSD). The three chromatograms are labeled with sample names for wild type grown in the presence of nitrogen (WT N+), an SN03 overexpression line (SN03), and wild type grown in the absence of nitrogen (WT N−). Groups of peaks representing classes of molecules are labeled at the bottom of the traces (Chlorphylides, Polar Lipids, Pheophytins and TAGs) and the chlorophyll-A (Chl-A) and chlorophyll B (Chl-B) peaks are labeled at top. The y-axis is the ELSD signal representing abundance and the x axis is HPLC column retention time (in minutes).

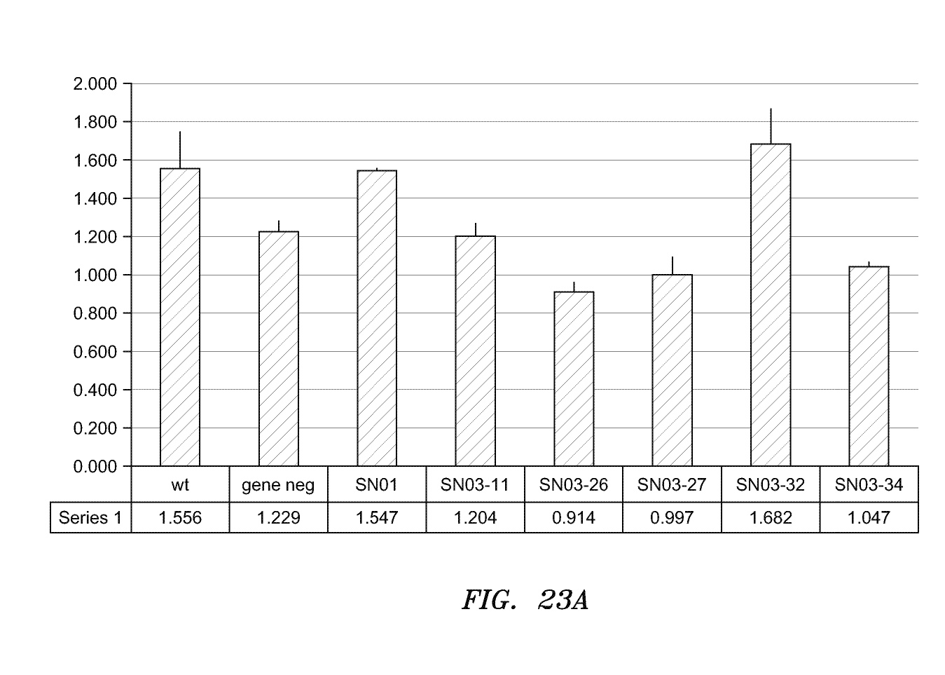

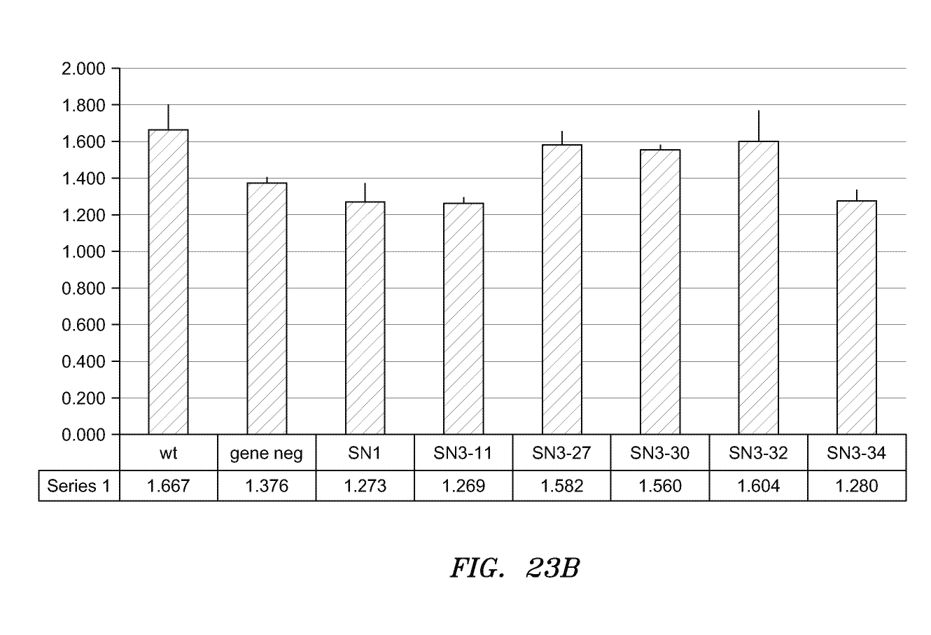

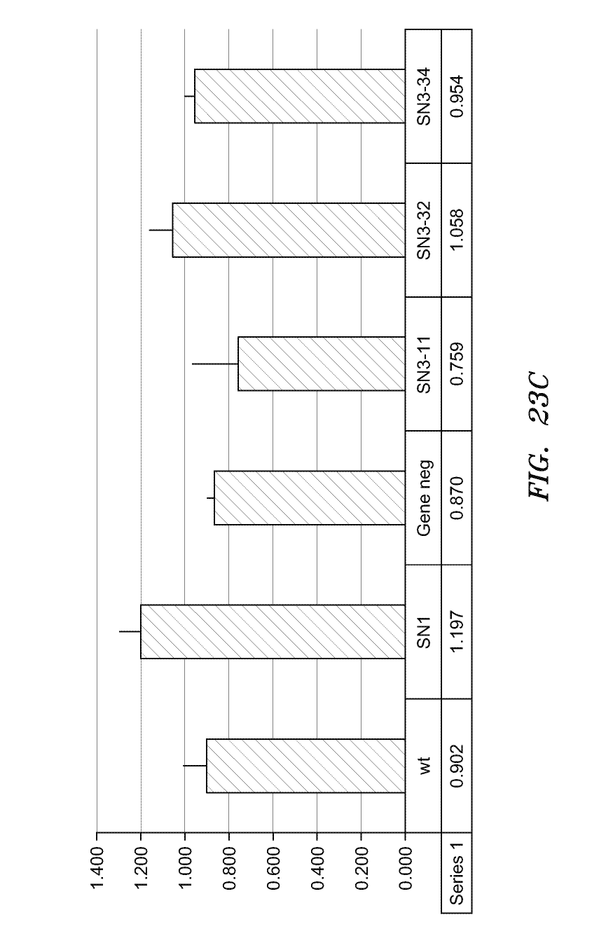

Growth rates in three SN03 over expression lines do not show notable differences relative to wild type, whether grown in TAP or HSM media. FIGS. 23A and B show growth rates of five different SN03 over expression lines grown in TAP media in an air environment under constant light as compared to a transgenic line that does not contain an SN gene (gene neg), one SN01 transgenic line and wild type. FIG. 23C shows the growth rate of three SN03 over expression lines grown in HSM media in a 5% carbon dioxide in air environment under constant light as compared to a transgenic line that does not contain an SN gene (gene neg), one SN01 transgenic line and wild type. Triplicates were grown for 4 to 5 days in 5 ml tubes on a rotating shaker. Optical density at 750 nm was taken 1-2 times a day and the growth rate was calculated as the slope of the linear portion of the growth curve based on the natural logarithm of the measured OD. This growth rate is shown on the y axis. The x axis represents the different lines used.

FIG. 45B is an additional example showing that growth rates in SN03 overexpression lines are comparable to wild type. Wild type Chlamydomonas reinhardtii and one SN03 over expression line were grown in 10-100 mL HSM media in a 5% carbon dioxide in air environment under constant light to mid log phase. Cells were diluted 1:100 into 12 to 24 wells of a 96-well plate containing 200 uL of HSM. The cells were grown in a 5% carbon dioxide in air environment under constant light to mid log phase. Optical density at 750 nm was taken 1-2 times a day and the growth rate was calculated as the slope of the linear portion of the growth curve based on the natural logarithm of the measured OD. This growth rate is shown on the y axis. The x axis represents the different strains used.

FIG. 45C shows that the carrying capacity of an SN03 overexpression line is similar to wild type. Wild-type Chlamydomonas reinhardtii cells and an SN03 overexpression line were grown in 0.5-2.0 L of HSM media in a 5% carbon dioxide in an air environment under constant light, until cells reached early log phase. The culture was centrifuged at 3000 to 5000×g for 5-10 minutes and one half of the culture was washed with 100-500 mL HSM, the other half with 100-500 mL HSM containing no nitrogen. After re-centrifugation, the two cultures were resuspended in a volume of media (HSM or HSM containing no nitrogen) equivalent to the starting culture volume. Cells were then grown in a 5% carbon dioxide in an air environment under constant light, until cells reached early stationary phase. 15 mL of culture was harvested by centrifugation and ash-free dry weight (AFDW) was determined. The AFDW in g/L is shown on the y-axis and the x-axis represents the lines used. Carrying capacity of the SN03 line is similar to wild type in the presence of nitrogen, and is reduced for both wild type and the SN03 overexpression line when grown in the absence of nitrogen.

FIG. 45D shows that total chlorophyll levels are comparable in wild type and an SN03 overexpression line, and that both wild type and the SN03 overexpression line have decreased chlorophyll when grown in the absence of nitrogen. Wild-type Chlamydomonas reinhardtii cells and an SN03 overexpression line were grown in 50-500 mL of HSM media in a 5% carbon dioxide in an air environment under constant light, until cells reached early log phase. The culture was centrifuged at 3000 to 5000×g for 5-10 minutes and one half of the culture was washed with 10-100 mL HSM, the other half with 10-100 mL HSM containing no nitrogen. After re-centrifugation, the two cultures were resuspended in a volume of media (HSM or HSM containing no nitrogen) equivalent to the starting culture volume. Cells were then grown in a 5% carbon dioxide in an air environment under constant light for an additional two days. 1-2 mL of culture was harvested by centrifugation. Cells were extracted in methanol and chlorophyll levels were determined spectroscopically as described in (LICHTENTHALER. Chlorophylls and Carotenoids: Pigments of Photosynthetic Biomembranes. Meth Enzymol (1987) vol. 148 pp. 350-382). Optical density (OD) of the culture at 750 nm was used to normalize to cell density. Chlorophyll levels are shown on the y axis and the x-axis represents the lines used.

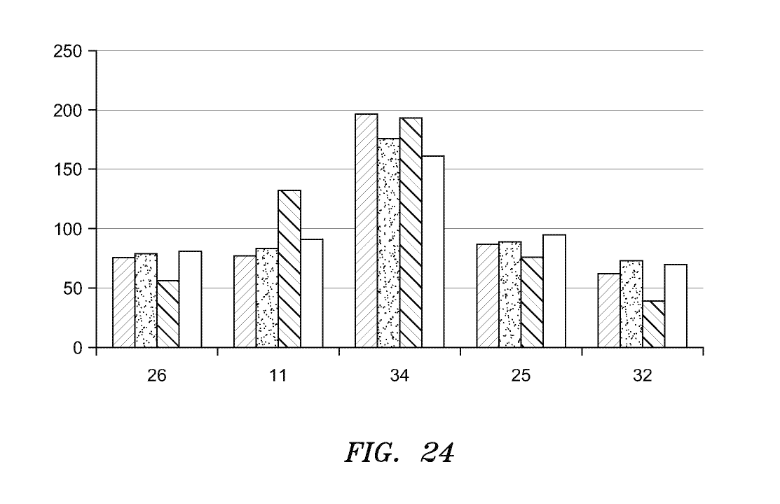

FIG. 24 shows that RNA is transcribed from the SN03 transgene. Wild-type Chlamydomonas reinhardtii cells as well as 5 SN03 overexpression lines were grown in 100-500 mL of TAP media in an air environment under constant light, until cells reached early log phase. Total RNA was prepared from wild type and 5 SN03 overexpression lines. 0.25-1.0 ug of RNA was used for iScript eDNA synthesis (BioRad, USA) and standard qPCR using iQ SybrGreen (BioRad, USA) detection was performed. Relative RNA levels were determined by qPCR using primers that amplify the SN03 transgene (four separate primer sets: SN03-1,2,3,4, represented by the four columns of each set in FIG. 24 (SEQ ID NOs: 24-31). Standard qPCR using SybrGreen detection was performed using Chlamydomonas reinhardtii ribosomal protein L11 for normalization between samples. Primers specific for the L11 RNA are SEQ ID NOs: 22 and 23. RNA levels on the y axis are relative to the average SN03 expression (levels in each of the five lines are normalized to an average of 100). The transgene was codon optimized for nuclear expression in Chlamydomonas reinhardtii so the endogenous gene was not detected. There is some variation amongst the different transgenic lines, but overall the absolute level of expression is high across the board (based on subjective assessment of Ct value in qPCR). The x-axis represents the SN03 overexpression strains (i.e. 26=SN03-26, 11=SN03-11, etc).

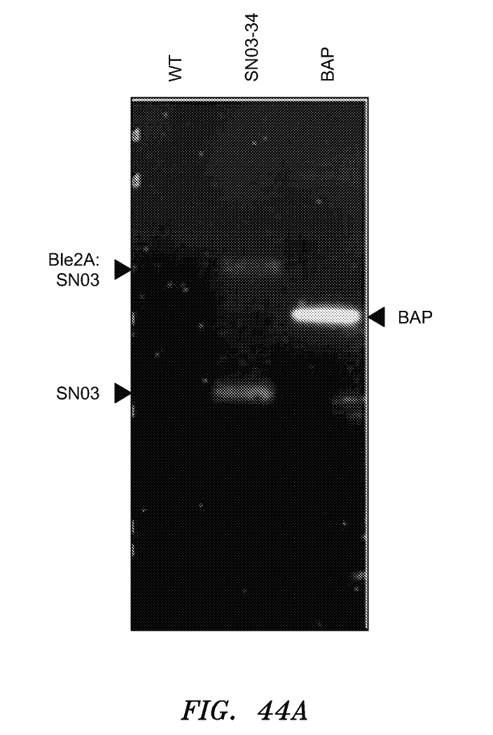

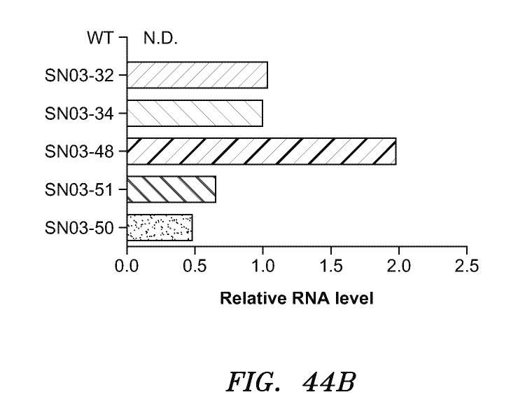

FIG. 44B is an additional example showing that RNA is transcribed from the SN03 transgene. Wild-type Chlamydomonas reinhardtii cells as well as 5 SN03 overexpression lines were grown in 100-500 mL of TAP media in an air environment under constant light, until cells reached early log phase. Total RNA was prepared from wild type and 5 SN03 overexpression lines. 0.25-1.0 ug of RNA was used for iScript cDNA synthesis (BioRad, USA) and standard qPCR using iQ SybrGreen (BioRad, USA) detection was performed. Relative RNA levels were determined by qPCR using primers that amplify the SN03 transgene. Standard qPCR using SybrGreen detection was performed using Chlamydomonas reinhardtii ribosomal protein L11 for normalization between samples. RNA levels on the x axis are relative to the expression of an average SN03 line (levels in each of the five lines are normalized to the level in line SN03-34 which was set to 1.0). The transgene was codon optimized for nuclear expression in Chlamydomonas reinhardtii so the endogenous gene was not detected. There is some variation amongst the different transgenic lines, but overall the absolute level of expression is high across the board (based on subjective assessment of Ct value in qPCR). The y-axis represents the SN03 overexpression strains.

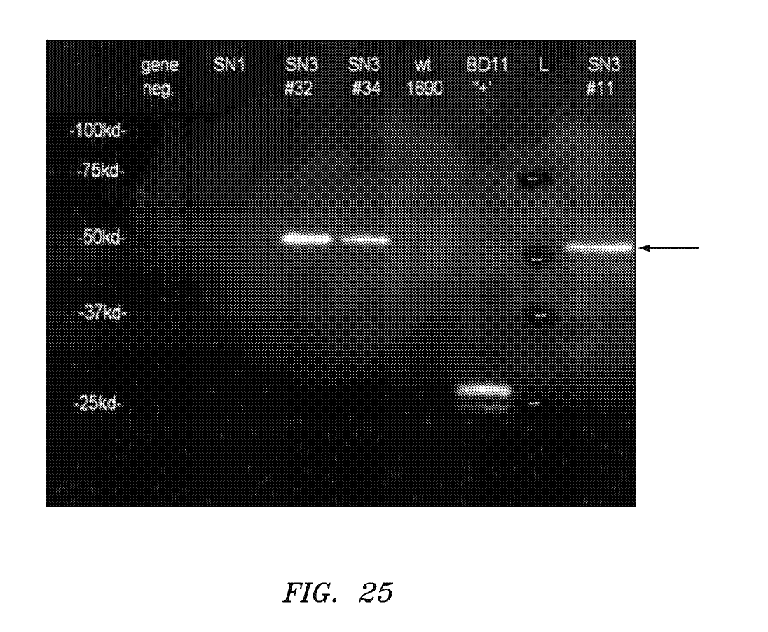

FIG. 25 shows that the SN03 protein (42 kDa) is detected in SN03 overexpression lines. Three of the SN03 transgenic lines along with a transgenic line that does not contain an SN gene (gene neg), one SN01 transgenic line and wild type were grown in 50-200 mL of TAP, centrifuged at 3000 to 5000×g for 5-10 minutes and prepared for Western immunoblotting. The SN03 protein has a FLAG-MAT tag attached. A strain overexpressing BD11 (xylanase) with a FLAG-MAT tag attached was used as a positive control. An antibody against FLAG was used to detect the tagged proteins after the samples were pulled down with a nickel column, run on SDS-PAGE and transferred to a nylon membrane. SN3 #32, SN3 #34, and SN3 #11 show a band at the correct size for the SN03 protein. The BD11 positive control is detected as well.

FIG. 44A is an additional example showing that the SN03 protein (42 kDa) is detected in an SN03 overexpression line. One SN03 overexpression line along with wild type was grown in 50-200 mL of TAP, centrifuged at 3000 to 5000×g for 5-10 minutes and prepared for Western immunoblotting. The SN03 protein has a FLAG-MAT tag attached. A bacterial alkaline phosphatase protein (BAP) with a FLAG-MAT tag attached was used as a positive control. An antibody against FLAG was used to detect the tagged proteins after the samples were pulled down with a nickel column, run on SDS-PAGE and transferred to a nylon membrane. The SN03-34 line shows two bands. The upper band is a fusion of bleomycin binding protein with SN03 protein connected by the 2A peptide. The lower band is the SN03 protein alone. The presence of the 2A mediated fusion protein has been described previously (Donnelly et al. Analysis of the aphthovirus 2A/2B polyprotein ‘cleavage’ mechanism indicates not a proteolytic reaction, but a novel translational effect: a putative ribosomal ‘skip’. J Gen Virol (2001) vol. 82 (Pt 5) pp. 1013-25). The BAP positive control is detected as well.

### Example 7

### RNA Transcriptomics of SN03 Transgenic Lines and Identification of Additional Nitrogen Stress Related Genes

Nitrogen starvation results in gene expression changes in Chlamydomonas, some subset of which is responsible for the increased lipid phenotype observed. SN03, as a putative transcription factor, is upregulated upon nitrogen starvation, and is likely involved in controlling some of the gene expression changes. Over expression of SN03 resulted in the increased lipid phenotype. Therefore, we are investigating the corresponding gene expression levels in transgenic cell lines over expressing SN03. We expect that the genes whose expression is modified by over expression of the SN03 transgene will be a subset of the genes affected by nitrogen starvation. This data will help us understand what downstream pathways the SN03 protein is acting upon to produce more lipid.

Three Chlamydomonas reinhardtii lines overexpressing SN03 were grown in 0.5-2 L of HSM media in a 5% carbon dioxide in an air environment under constant light, until cells reached early log phase. 50-100 mL of the cells were harvested by centrifugation at 3000 to 5000×g for 5-10 minutes and RNA was purified from the cultures. This RNA was sequenced using standard Solexa methodologies (Sequensys, Inc, La Jolla, Calif.) for use in the RNA-Seq analysis method. Sequences were mapped to the JGI Chlamydomonas reinhardtii version 3.0 or version 4.0 transcriptome using Arraystar software (DNASTAR, USA). Presented below in Table 4 is the total number of Solexa 36 bp reads generated for each of the three RNA samples. Also shown for each sample are the number of those reads that successfully mapped to the Chlamydomonas reinhardtii transcriptome (total reads with mer hits) and the percentage of total hits mapped to the transcriptome.

TABLE 4

SN03-41
Total Sample reads: 17,308,430
Total reads with mer hits: 13,204,180
Percentage mapped: 76.3
SN03-48
Total Sample reads: 14,256,269
Total reads with mer hits: 10,669,978
Percentage mapped: 74.8
SN03-34
Total Sample reads: 11,885,067
Total reads with mer hits: 8,637,432
Percentage mapped: 72.7

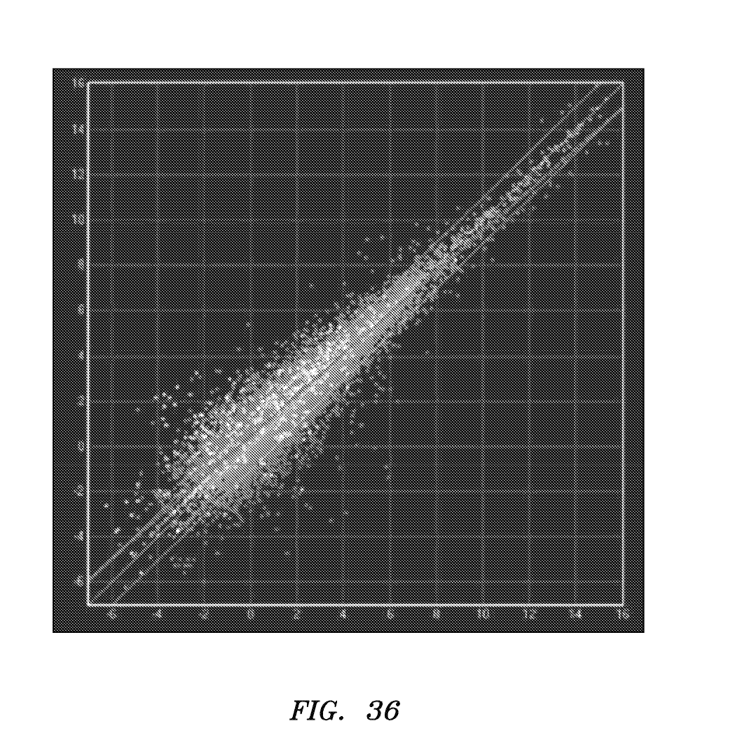

FIG. 36 shows a plot of all 16,000+ genes in SE0050 with expression levels from a different sample on each axis. Shown here are Exponential growth +Nitrogen (x-axis) versus Exponential growth 6 H −Nitrogen (y-axis). Genes with no change in expression level are on the diagonal; those above the diagonal are upregulated after 6 hours of nitrogen starvation and those below the diagonal are down regulated after 6 hours of nitrogen starvation. The white data points represent at least 4-fold increase in expression in one SN03 overexpression line relative to wild type. Many of the genes that are upregulated in the SN03 overexpression line are also upregulated after 6 hours of nitrogen starvation (shown by the white dots above the diagonal). However, there are some genes that are up regulated in the SN03 overexpression line while also down regulated after 6 hours of nitrogen starvation (shown by white dots below the diagonal).

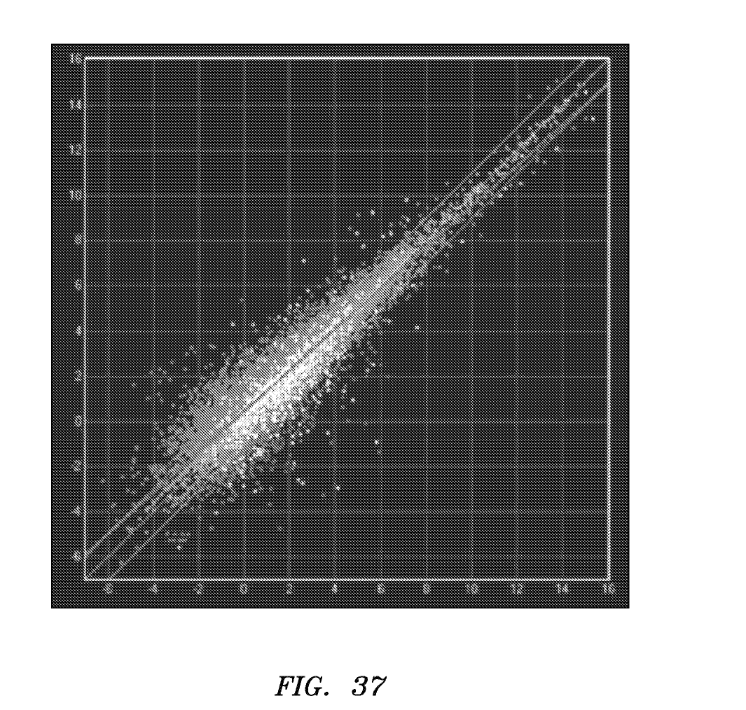

FIG. 37 shows a plot of all 16,000+ genes in SE0050 with expression levels from a different sample on each axis. Shown here are Exponential growth +Nitrogen (x-axis) versus Exponential growth 6 H −Nitrogen (y-axis). Genes with no change in expression level are on the diagonal; those above the diagonal are upregulated after 6 hours of nitrogen starvation and those below the diagonal are down regulated after 6 hours of nitrogen starvation. The white data points represent at least 4-fold decrease in expression in one SN03 overexpression line relative to wild type. Many of the genes that are down regulated in the SN03 overexpression line are also down regulated after 6 hours of nitrogen starvation (shown by the white dots below the diagonal). However, there are some genes that are down regulated in the SN03 overexpression line while also up regulated after 6 hours of nitrogen starvation (shown by white dots above the diagonal).

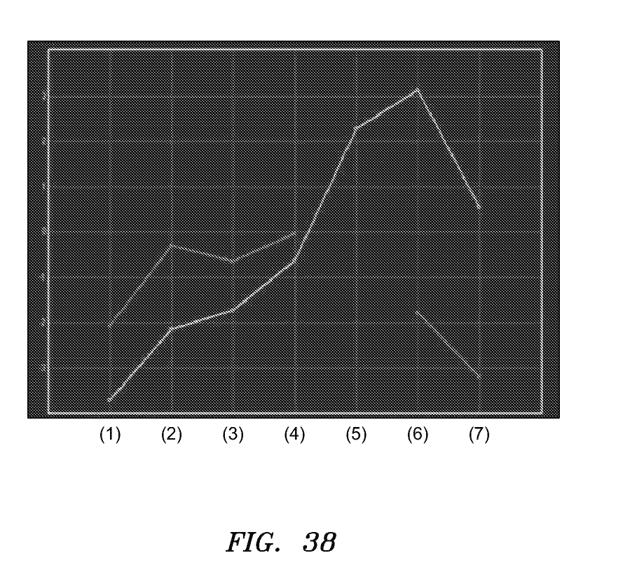

FIG. 38 shows RNA levels for the endogenous SN03 transcript and the transgenic SN03 transcript. Expression level (shown on y axis in log 2 scale) was determined by the DNASTAR Arraystar software from the RNA-Seq data on a time course of nitrogen starved wild type Chlamydomonas reinhardtii and three SN03 overexpression lines (strains and conditions indicated on x axis). Because the endogenous and transgenic SN03 sequences are similar but not identical (due to codon optimization), the Arraystar software cannot assign reads to the transcripts with 100% accuracy. The transgenic SN03 transcript is not present in the wild type samples as shown by the low expression levels indicated for the wild type samples and the high levels in the SN03 overexpression lines. Induction of endogenous SN03 expression upon nitrogen starvation is demonstrated here in the nitrogen starved wild type samples.

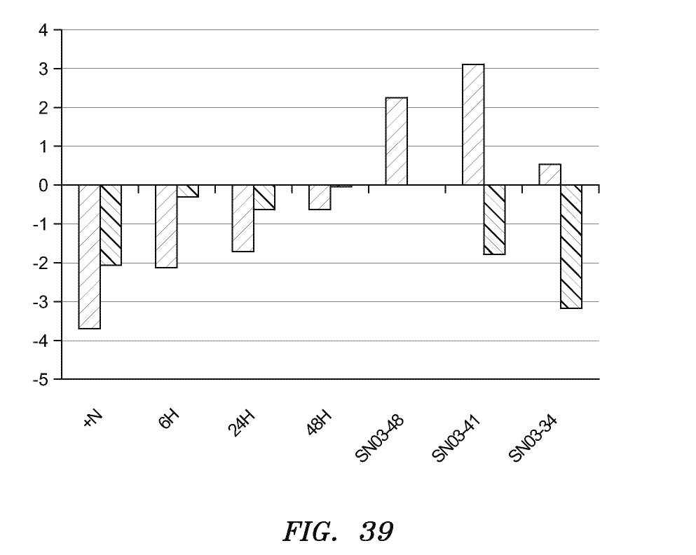

FIG. 39 shows RNA levels for the endogenous SN03 transcript and the transgenic SN03 transcript, as in FIG. 38. The y axis shows the RNA expression level (log 2 scale) and each set of two columns represents the strains and conditions used. The left column in each set is the expression level of the transgenic SN03 RNA and the right column in each set is the expression level of the endogenous SN03 RNA. The transgenic SN03 transcript is not present in the wild type samples as shown by the low expression levels indicated for the wild type samples and the high levels in the SN03 overexpression lines. Induction of endogenous SN03 expression upon nitrogen starvation is demonstrated here in the nitrogen starved wild type samples.

This RNA-Seq data is used to identify candidate gene lists for further understanding the impact of SN03 overexpression and for additional target gene identification. Solexa sequenced RNA from a nitrogen starved time course of wild type Chlamydomonas reinhardtii, described above in EXAMPLE 3, and from three SN03 overexpression lines was mapped to the JGI Chlamydomonas reinhardtii transcriptome using DNASTAR Arraystar.

Using Arraystar software, sets of genes with relevant expression patterns were identified. 235 genes were identified that were at least 4 fold up regulated in one or more nitrogen starvation sample as well as at least 4 fold up regulated in at least one SN03 overexpression strain. 191 genes were identified that were at least 4 fold down regulated in one or more nitrogen starvation sample as well as at least 4 fold down regulated in at least one SN03 overexpression strain. 134 genes were identified that were at least 4 fold up regulated in one or more nitrogen starvation sample as well as at least 4 fold down regulated in at least one SN03 overexpression strain. 38 genes were identified that were at least 4 fold down regulated in one or more nitrogen starvation sample as well as at least 4 fold up regulated in at least one SN03 overexpression strain.

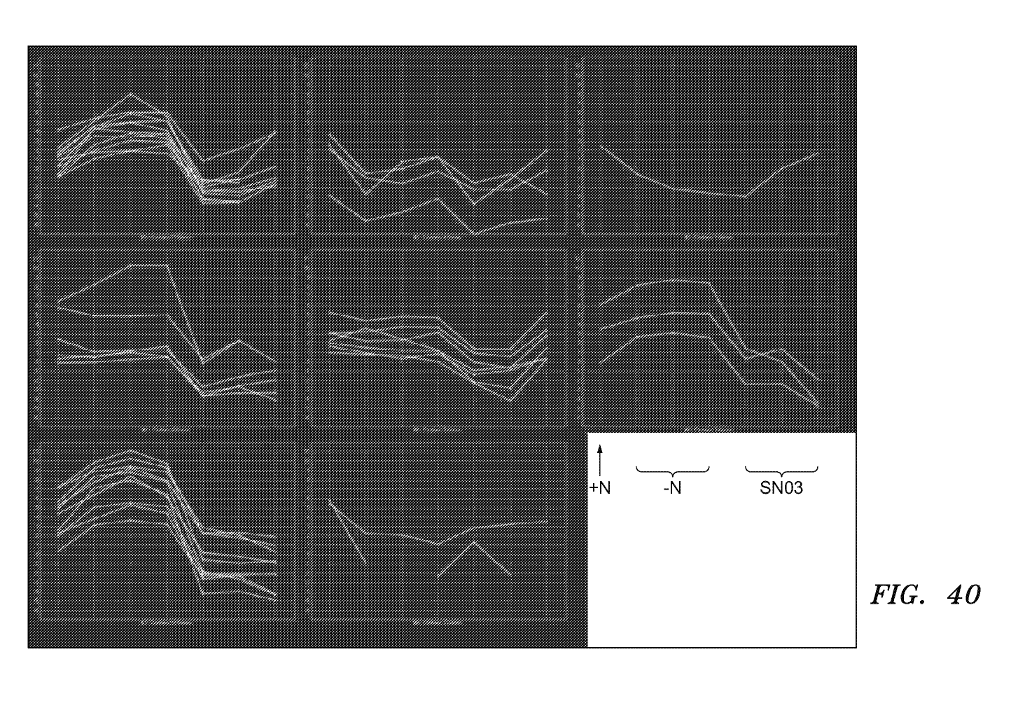

An additional way to analyze the RNA-Seq data is shown in FIG. 40. This figure shows the dynamics of gene expression during nitrogen starvation (Exponential +nitrogen and 6 H, 24 H, 48 H-nitrogen) and in three SN03 overexpression strains. Each line represents one gene, with the y axis in each case being the level of expression and the x axis representing the 7 sequenced samples. The eight graphs represent genes that have similar expression patterns across the conditions represented by the 7 samples. Most of the graphs here represent sets of genes that are upregulated by nitrogen starvation but that are not upregulated by SN03 overexpression.

As examples of the genes that can be identified by this approach, at least five known genes with a KOG functional annotation of Histone protein (either Histone H2B or Histone H3 and H4) are shown to be up and/or down regulated by both nitrogen starvation and SN03 overexpression. These are examples of expression patterns derived from SN03 overexpression lines that can be used to understand the nitrogen starvation pathways. These genes and their expression patterns are as follows: JGI protein ID 97703: 9 fold up in nitrogen starvation, 82 fold up in SN03 overexpression line; JGI protein ID 170323: 89 fold up in nitrogen starvation, 40 fold up in SN03 overexpression line; JGI protein ID 115268: 5 fold down in nitrogen starvation, 45 fold down in SN03 overexpression line; JGI protein ID 167094: 79 fold down in nitrogen starvation, 22 fold down in SN03 overexpression line; and JGI protein ID 100008: 4 fold up in nitrogen starvation, 9 fold down in SN03 overexpression line.

One hundred and one genes (including SN03) were identified as candidates for overexpression in Chlamydomonas reinhardtii, based on expression patterns in nitrogen starvation. The genes selected showed at least a four-fold increase in expression in one or more of the nitrogen starvation time points. These expression patterns are shown in Table 5.

| | | | |
|---|---|---|---|
| Gene | Nitrogen 6 H | Nitrogen 24 H | Nitrogen 48 H |

| | | | | | | |
|---|---|---|---|---|---|---|
| SN01 | 88.752 | up | 15.531 | up | 62.340 | up |
| SN02 | 41.497 | up | 37.269 | up | 36.091 | up |
| SN03 | 41.264 | up | 30.110 | up | 29.339 | up |
| SN04 | 31.458 | up | 11.010 | up | 17.677 | up |
| SN05 | 52.070 | up | 67.896 | up | 51.691 | up |
| SN06 | 287.371 | up | 441.829 | up | 259.971 | up |
| SN07 | 18.037 | up | 12.886 | up | 12.791 | up |
| SN08 | 7.309 | up | 5.075 | up | 10.000 | up |
| SN09 | 5.066 | up | 11.644 | up | 7.857 | up |
| SN10 | 6.966 | up | 8.677 | up | 6.383 | up |
| SN11 | 5.913 | up | 31.364 | up | 20.842 | up |
| SN12 | 14.575 | up | 8.589 | up | 16.036 | up |
| SN13 | 13.173 | up | 25.081 | up | 9.285 | up |
| SN14 | 17.778 | up | 17.915 | up | 21.579 | up |
| SN15 | 30.605 | up | 12.024 | up | 4.794 | up |
| SN16 | 11.456 | up | 18.052 | up | 10.770 | up |
| SN17 | 5.066 | up | 4.478 | up | 5.714 | up |
| SN18 | 15.940 | up | 49.319 | up | 22.473 | up |
| SN19 | 7.853 | up | 7.263 | up | 6.517 | up |
| SN20 | 114.541 | up | 108.572 | up | 178.571 | up |
| SN21 | 6.920 | up | 8.556 | up | 10.075 | up |
| SN22 | 57.203 | up | 90.071 | up | 23.653 | up |
| SN23 | 7.245 | up | 6.454 | up | 6.456 | up |
| SN24 | 1474.950 | up | 593.660 | up | 1.179 | down |
| SN25 | 216.831 | up | 460.015 | up | 305.683 | up |
| SN26 | 291.979 | up | 3.249 | down | 1.179 | down |
| SN27 | 5.991 | up | 11.728 | up | 5.190 | up |
| SN28 | 12.447 | up | 11.003 | up | 8.774 | up |
| SN29 | 11.202 | up | 83.572 | up | 34.765 | up |
| SN30 | 13.173 | up | 4.478 | up | 7.142 | up |
| SN31 | 9.119 | up | 8.061 | up | 6.428 | up |
| SN32 | 6.789 | up | 18.005 | up | 33.501 | up |
| SN33 | 16.603 | up | 24.461 | up | 14.230 | up |
| SN34 | 12.499 | up | 6.443 | up | 5.714 | up |
| SN35 | 18.642 | up | 16.479 | up | 4.380 | up |
| SN36 | 23.312 | up | 13.738 | up | 10.955 | up |
| SN37 | 545.960 | up | 202.386 | up | 37.242 | up |
| SN38 | 5.964 | up | 4.853 | up | 4.919 | up |
| SN39 | 23.306 | up | 31.351 | up | 37.857 | up |
| SN40 | 7.093 | up | 20.026 | up | 14.285 | up |
| SN41 | 6.305 | up | 4.279 | up | 6.428 | up |
| SN42 | 274.981 | up | 121.538 | up | 323.051 | up |
| SN43 | 454.842 | up | 185.401 | up | 165.816 | up |
| SN44 | 9.119 | up | 12.540 | up | 5.312 | up |
| SN45 | 10.900 | up | 9.635 | up | 15.366 | up |
| SN46 | 70.277 | up | 14.671 | up | 81.893 | up |
| SN47 | 8.673 | up | 23.000 | up | 6.113 | up |
| SN48 | 395.398 | up | 279.617 | up | 222.969 | up |
| SN49 | 21.115 | up | 46.663 | up | 14.884 | up |
| SN50 | 6.055 | up | 16.059 | up | 25.611 | up |
| SN51 | 4.190 | up | 4.310 | up | 10.541 | up |
| SN52 | 9.292 | up | 4.117 | up | 11.058 | up |
| SN53 | 18.773 | up | 16.594 | up | 15.438 | up |
| SN54 | 4.053 | up | 4.926 | up | 4.285 | up |
| SN55 | 9.307 | up | 6.270 | up | 7.857 | up |
| SN56 | 10.639 | up | 17.019 | up | 14.285 | up |
| SN57 | 2.154 | down | 78.354 | up | 31.240 | up |
| SN58 | 6.810 | up | 7.804 | up | 4.051 | up |
| SN59 | 11.667 | up | 3.249 | down | 1.179 | down |
| SN60 | 153.284 | up | 27.734 | up | 7.496 | up |
| SN61 | 10.745 | up | 21.220 | up | 44.479 | up |
| SN62 | 4.693 | up | 1.791 | up | 2.515 | up |
| SN63 | 2.154 | down | 15.987 | up | 12.748 | up |
| SN64 | 2.020 | up | 5.778 | up | 3.952 | up |
| SN65 | 2.364 | up | 3.390 | up | 9.523 | up |
| SN66 | 5.066 | up | 3.583 | up | 7.142 | up |
| SN67 | 23.051 | up | 12.422 | up | 13.675 | up |
| SN68 | 8.106 | up | 10.338 | up | 10.386 | up |
| SN69 | 13.582 | up | 13.037 | up | 9.835 | up |
| SN70 | 180.585 | up | 212.843 | up | 127.292 | up |
| SN71 | 2.154 | down | 14.433 | up | 11.509 | up |
| SN72 | 14.630 | up | 25.865 | up | 61.875 | up |
| SN73 | 162.405 | up | 239.269 | up | 76.318 | up |
| SN74 | 20.629 | up | 9.117 | up | 1.179 | down |
| SN75 | 7.600 | up | 1.343 | up | 1.071 | up |
| SN76 | 4.446 | up | 11.433 | up | 4.714 | up |
| SN77 | 4.867 | up | 10.732 | up | 4.271 | up |
| SN78 | 180.813 | up | 3.249 | down | 1.179 | down |
| SN79 | 72.681 | up | 107.626 | up | 64.366 | up |
| SN80 | 57.203 | up | 90.071 | up | 23.653 | up |
| SN81 | 51.267 | up | 60.425 | up | 24.092 | up |
| SN82 | 47.870 | up | 3.249 | down | 8.435 | up |
| SN83 | 41.743 | up | 34.061 | up | 1.179 | down |
| SN84 | 34.438 | up | 14.433 | up | 13.134 | up |
| SN85 | 33.749 | up | 52.208 | up | 11.894 | up |
| SN86 | 30.210 | up | 3.249 | down | 3.549 | up |
| SN87 | 21.092 | up | 11.184 | up | 1.179 | down |
| SN88 | 13.173 | up | 9.853 | up | 2.857 | up |
| SN89 | 11.724 | up | 41.454 | up | 8.264 | up |
| SN90 | 11.711 | up | 5.151 | up | 8.216 | up |
| SN91 | 11.146 | up | 1.116 | down | 1.428 | up |
| SN92 | 11.146 | up | 9.853 | up | 2.142 | up |
| SN93 | 10.421 | up | 3.249 | down | 1.179 | down |
| SN94 | 8.444 | up | 5.075 | up | 8.809 | up |
| SN95 | 8.294 | up | 4.360 | up | 1.463 | up |
| SN96 | 7.155 | up | 5.862 | up | 2.516 | up |
| SN97 | 7.093 | up | 1.116 | down | 1.428 | up |
| SN98 | 7.061 | up | 10.690 | up | 8.524 | up |
| SN99 | 6.966 | up | 8.677 | up | 6.383 | up |
| SN100 | 6.766 | up | 5.981 | up | 1.179 | down |
| SN101 | 6.079 | up | 1.194 | up | 1.377 | down |

In addition, thirty genes were identified as candidates for overexpression in Chlamydomonas reinhardtii, based on the expression patterns in nitrogen starvation and SN03 overexpression. The genes selected showed at least a four-fold increase in expression in both of the SN03 overexpression Lines (SN03-4 and SN3-41). These expression levels are shown in Table 6.

| Gene | Nitrogen 6 H | | Nitrogen 24 H | | Nitrogen 48 H | | SN03-48 | | SN03-41 | |
|---|---|---|---|---|---|---|---|---|---|---|
| SN108 | 9.261 | up | 2.877 | up | 1.931 | up | 16.278 | up | 17.199 | up |
| SN109 | 6.615 | up | 15.740 | up | 17.379 | up | 10.359 | up | 14.826 | up |
| SN110 | 14.904 | up | 11.820 | up | 9.426 | up | 6.668 | up | 13.361 | up |
| SN111 | 4.145 | up | 26.234 | up | 3.862 | up | 76.718 | up | 5.930 | up |
| SN112 | 17.861 | up | 7.870 | up | 8.689 | up | 1.479 | up | 8.006 | up |
| SN113 | 10.617 | up | — | | 4.827 | up | 13.505 | up | 11.861 | up |
| SN114 | 24.279 | up | 1.899 | up | 72.957 | up | 70.989 | up | 54.366 | up |
| SN115 | 5.953 | up | 7.214 | up | 4.344 | up | 13.689 | up | 13.047 | up |
| SN116 | 34.257 | up | — | | 13.490 | up | 11.551 | up | 8.690 | up |
| SN117 | 29.699 | up | 22.489 | up | 2.071 | down | 28.646 | up | 16.775 | up |
| SN118 | 10.066 | up | 15.523 | up | 8.978 | up | 77.593 | up | 41.444 | up |
| SN119 | 3.806 | up | 6.343 | up | 3.621 | up | 6.894 | up | 12.803 | up |
| SN120 | 3.528 | up | 12.242 | up | 5.149 | up | 14.799 | up | 14.233 | up |
| SN121 | 11.311 | up | 90.343 | up | 1.989 | up | 33.617 | up | 8.820 | up |
| SN122 | 9.468 | up | 1.750 | up | 2.416 | up | 40.808 | up | 25.817 | up |
| SN123 | 5.292 | up | 7.870 | up | 5.793 | up | 8.139 | up | 7.710 | up |
| SN124 | 6.363 | up | 5.996 | up | 5.149 | up | 4.263 | up | 5.140 | up |
| SN125 | 10.584 | up | 6.558 | up | 3.247 | up | 12.126 | up | 21.426 | up |
| SN126 | 5.292 | up | 13.773 | up | 11.586 | up | 8.509 | up | 8.006 | up |
| SN127 | 7.817 | up | 1.475 | up | 7.016 | up | 21.317 | up | 48.514 | up |
| SN128 | 5.408 | up | 113.889 | up | 71.350 | up | 105.014 | up | 106.190 | up |
| SN129 | 2.667 | up | 7.836 | up | 5.287 | up | 9.475 | up | 6.685 | up |
| SN130 | 3.969 | up | 5.246 | up | 6.758 | up | 18.683 | up | 22.536 | up |
| SN131 | 65.608 | up | 164.232 | up | 125.693 | up | 549.544 | up | 281.672 | up |
| SN132 | 7.938 | up | 3.935 | up | 1.931 | up | 13.319 | up | 13.640 | up |
| SN133 | 44.134 | up | 1.543 | up | — | | 40.422 | up | 38.763 | up |
| SN134 | 9.261 | up | 1.311 | up | 1.931 | up | 13.319 | up | 24.909 | up |
| SN135 | 1.323 | up | — | | 4.352 | up | 82.500 | up | 55.156 | up |
| SN136 | 7.274 | up | 6.198 | up | 5.790 | up | 7.728 | up | 22.525 | up |

| | | | | | | | | | |
|---|---|---|---|---|---|---|---|---|---|
| SN137 | 5.139 | up | 5.199 | up | 3.835 | up | 22.281 | up | 17.276 |

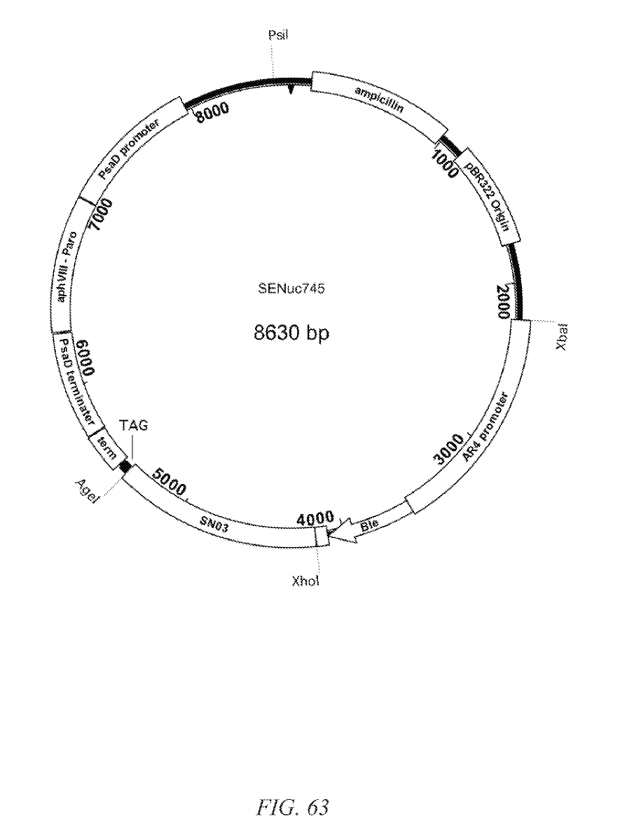

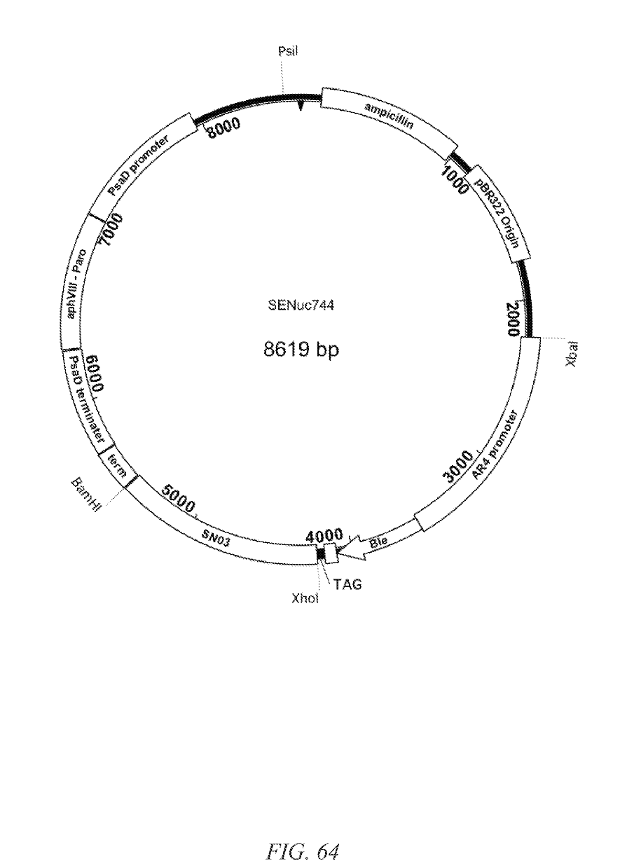

The ORFs for these one hundred and thirty one stress response targets (described in the table below) were each codon optimized using Chlamydomonas reinhardtii nuclear codon usage tables, and synthesized. The DNA constructs for the 131 targets were individually cloned into nuclear overexpression vector Ble2A (as shown in FIG. 34, FIG. 63, or FIG. 64) and transformed into SE0050. This construct results in the production of one RNA with a nucleotide sequence encoding a selection protein (Ble) and a nucleotide sequence encoding a protein of interest (any one of SN01 to SN137). The expression of the two proteins are linked by the viral peptide 2A (for example, as described in Donnelly et al., J Gen Virol (2001) vol. 82 (Pt 5) pp. 1013-25). This protein sequence facilitates expression of two polypeptides from a single mRNA. The 131 genes are described below in Table 7. A sequence identifier is also provided for several of the genes.

TABLE 7

| | | Vector | |
|---|---|---|---|
| Gene | JGI PID | Used | KOG define |
| SN01 | 179214 | FIG. 34 | Translation initiation factor 4F, |
| | | | ribosome/mRNA-bridging subunit |
| | | | (eIF-4G) |
| SN02 | 151215 | FIG. 34 | HMG box-containing protein |
| SN03 | 147817 | FIG. 34 | CREB binding protein/P300 and |
| | | | related TAZ Zn-finger proteins |
| SN04 | 141971 | FIG. 34 | Transcription factor CHX10 and |
| | | | related HOX domain proteins |
| SN05 | 168511 | FIG. 34 | |
| SN06 | 295492 | FIG. 63 | |
| SN07 | 152866 | FIG. 64 | Chitinase |
| SN08 | 149064 | FIG. 63 | HMG-box transcription factor |
| SN09 | 286781 | FIG. 64 | Nuclear receptor coregulator |
| | | | SMRT/SMRTER, contains Myb-like |
| | | | domains |
| SN10 | 148696 | FIG. 64 | Nuclear pore complex, Nup98 |
| | | | component (sc |
| | | | Nup145/Nup100/Nup116) |
| SN11 | 289473 | FIG. 64 | CREB binding protein/P300 and |
| | | | related TAZ Zn-finger proteins |
| SN12 | 287564 | FIG. 63 | Nuclear receptor coregulator |
| | | | SMRT/SMRTER, contains Myb-like |
| | | | domains |
| SN13 | 152791 | FIG. 63 | Nuclear receptor coregulator |
| | | | SMRT/SMRTER, contains Myb-like |
| | | | domains |
| SN14 | 426054 | FIG. 64 | Nuclear receptor coregulator |
| | | | SMRT/SMRTER, contains Myb-like |
| | | | domains |
| SN15 | 150878 | FIG. 64 | Nuclear receptor coregulator |
| | | | SMRT/SMRTER, contains Myb-like |
| | | | domains |
| SN16 | 282597 | FIG. 63 | Transcription initiation, factor TFIID, |
| | | | subunit BDF1 and related bromodomain |
| | | | proteins |
| SN17 | 174292 | FIG. 63 | E3 ubiquitin-protein ligase/Putative |
| | | | upstream regulatory element binding |
| | | | protein |
| SN18 | 169885 | FIG. 64 | Transcription initiation factor TFIID, |
| | | | subunit BDF1 and related bromodomain |
| | | | proteins |
| SN19 | 327993 | FIG. 64 | Nuclear receptor coregulator |
| | | | SMRT/SMRTER, contains Myb-like |
| | | | domains |
| SN20 | 405949 | FIG. 64 | Nuclear receptor coregulator |
| | | | SMRT/SMRTER, contains Myb-like |
| | | | domains |
| SN21 | 169264 | FIG. 64 | Xanthine/uracil transporters |
| SN22 | 196335 | FIG. 63 | Na+/Pi symporter |
| SN23 | 195838 | FIG. 63 | Nuclear receptor coregulator |
| | | | SMRT/SMRTER, contains Myb-like |
| | | | domains |
| SN24 | 285589 | FIG. 64 | |
| SN25 | 393275 | FIG. 64 | |
| SN26 | 382107 | FIG. 63 | |
| SN27 | 403062 | FIG. 64 | FOG: Zn-finger |
| SN28 | 291009 | FIG. 63 | Nuclear receptor coregulator |
| | | | SMRT/SMRTER, contains Myb-like |
| | | | domains |
| SN29 | 409462 | FIG. 63 | TATA box binding protein (TBP)- |
| | | | associated factor, RNA polymerase II |
| SN30 | 289999 | FIG. 64 | Nuclear receptor coregulator |
| | | | SMRT/SMRTER, contains Myb-like |
| | | | domains |
| SN31 | 390376 | FIG. 63 | C-type lectin |
| SN32 | 151559 | FIG. 64 | Transcription initiation factor TFIID, |
| | | | subunit BDF1 and related bromodomain |
| | | | proteins |
| SN33 | 406853 | FIG. 64 | Choline transporter |
| SN34 | 404335 | FIG. 64 | Nuclear receptor coregulator |
| | | | SMRT/SMRTER, contains Myb-like |
| | | | domains |
| SN35 | 286994 | FIG. 64 | Nuclear receptor coregulator |
| | | | SMRT/SMRTER, contains Myb-like |
| | | | domains |
| SN36 | 296096 | FIG. 63 | Triglyceride lipase-cholesterol esterase |
| SN37 | 338073 | FIG. 64 | Predicted alpha-helical protein, |
| | | | potentially involved in |
| | | | replication/repair |
| SN38 | 418372 | FIG. 63 | Signaling protein SWIFT and related |
| | | | BRCT domain proteins |
| SN39 | 303091 | FIG. 63 | Predicted membrane protein, contains |
| | | | DoH and Cytochrome b-561/ferric |
| | | | reductase transmembrane domains |
| SN40 | 205508 | FIG. 64 | Pyrazinamidase/nicotinamidase PNC1 |
| SN41 | 177225 | FIG. 64 | |
| SN42 | 297943 | FIG. 63 | |
| SN43 | 407911 | FIG. 63 | |
| SN44 | 342055 | FIG. 64 | |
| SN45 | 148736 | FIG. 64 | Runt and related transcription factors |
| SN46 | 293583 | FIG. 64 | Nuclear receptor coregulator |
| | | | SMRT/SMRTER, contains Myb-like |
| | | | domains |
| SN47 | 324824 | FIG. 63 | Transcription regulator dachshund, |
| | | | contains SKI/SNO domain |
| SN48 | 149352 | FIG. 63 | |
| SN49 | 393575 | FIG. 64 | Transcription initiation factor TFIID, |
| | | | subunit BDF1 and related bromodomain |
| | | | proteins |
| SN50 | 293934 | FIG. 63 | Transcription coactivator |
| SN51 | 291744 | FIG. 63 | Nuclear receptor coregulator |
| | | | SMRT/SMRTER, contains Myb-like |
| | | | domains |
| SN52 | 397925 | FIG. 64 | Nuclear receptor coregulator |
| | | | SMRT/SMRTER, contains Myb-like |
| | | | domains |
| SN53 | 289237 | FIG. 63 | Nuclear receptor coregulator |
| | | | SMRT/SMRTER, contains Myb-like |
| | | | domains |
| SN54 | 422537 | FIG. 63 | Transcription initiation factor TFIID, |
| | | | subunit BDF1 and related bromodomain |
| | | | proteins |
| SN55 | 338285 | FIG. 63 | Acetylglucosaminyltransferase |
| | | | EXT1/exostosin 1 |
| SN56 | 141561 | FIG. 64 | Membrane protein Patched/PTCH |
| SN57 | 121702 | FIG. 64 | Molecular chaperone (DnaJ |
| | | | superfamily) |
| SN58 | 182549 | FIG. 63 | |
| SN59 | 143030 | FIG. 63 | Conserved Zn-finger protein |
| SN60 | 283406 | FIG. 63 | |
| SN61 | 149068 | FIG. 64 | Conserved Zn-finger protein |
| SN62 | 144787 | FIG. 63 | CREB binding protein/P300 and |
| | | | related TAZ Zn-finger proteins |
| SN63 | 145290 | FIG. 63 | FOG: Zn-finger |
| SN64 | 289771 | FIG. 64 | CREB binding protein/P300 and |
| | | | related TAZ Zn-finger proteins |
| SN65 | 152247 | FIG. 63 | FOG: Zn-finger |
| SN66 | 290187 | FIG. 64 | FOG: Zn-finger |
| SN67 | 416754 | FIG. 63 | FOG: Zn-finger |
| SN68 | 191432 | FIG. 63 | Uroporphyrin III methyltransferase |
| SN69 | 158745 | FIG. 64 | Ammonia permease |
| SN70 | 147414 | FIG. 63 | |
| SN71 | 153527 | FIG. 64 | Nuclear receptor coregulator |
| | | | SMRT/SMRTER, contains Myb-like |
| | | | domains |
| SN72 | 422638 | FIG. 64 | Conserved Zn-finger protein |
| SN73 | 410505 | FIG. 64 | |
| SN74 | 296873 | FIG. 64 | FOG: Zn-finger |
| SN75 | 149959 | FIG. 64 | Transcription factor containing |
| | | | C2HC type Zn finger |
| SN76 | 192085 | FIG. 63 | Sulfite reductase (ferredoxin) |
| SN77 | 184660 | FIG. 63 | |
| SN78 | 295739 | FIG. 64 | SWI/SNF-related matrix-associated |
| | | | actin-dependent regulator of |
| | | | chromatin |
| SN79 | 423635 | FIG. 64 | Nuclear inhibitor of phosphatase-1 |
| SN80 | 196335 | FIG. 63 | Na+/Pi symporter |
| SN81 | 405943 | FIG. 64 | Predicted E3 ubiquitin ligase |
| SN82 | 337172 | FIG. 64 | Rho GTPase effector BNI1 and |
| | | | related formins |
| SN83 | 420539 | FIG. 63 | Histone acetyltransferase |
| | | | SAGA/ADA, catalytic subunit |
| | | | PCAF/GCN5 and related proteins |
| SN84 | 151805 | FIG. 63 | Uncharacterized conserved protein, |
| | | | contains BTB/POZ domain |
| SN85 | 20444 | FIG. 64 | Ankyrin |
| SN86 | 294811 | FIG. 64 | Dystonin, GAS (Growth-arrest- |
| | | | specific protein), and related |
| | | | proteins |
| SN87 | 333839 | FIG. 64 | Defense-related protein containing |
| | | | SCP domain |
| SN88 | 407214 | FIG. 64 | Reductases with broad range of |
| | | | substrate specificities |
| SN89 | 151874 | FIG. 63 | FOG: Leucine rich repeat |
| SN90 | 296678 | FIG. 63 | K+-channel ERG and related |
| | | | proteins, contain PAS/PAC sensor |
| | | | domain |
| SN91 | 399766 | FIG. 64 | von Willebrand factor and related |
| | | | coagulation proteins |
| SN92 | 327945 | FIG. 63 | Putative transcription factor |
| | | | HALR/MLL3, involved in embryonic |
| | | | development |
| SN93 | 158019 | FIG. 64 | Calcium-responsive transcription |
| | | | coactivator |
| SN94 | 291531 | FIG. 63 | ATP-dependent RNA helicase |
| SN95 | 285435 | FIG. 64 | Calcium-responsive transcription |
| | | | coactivator |
| SN96 | 411176 | FIG. 63 | Rac1 GTPase effector FRL |
| SN97 | 149339 | FIG. 63 | Fibrillarin and related nucleolar |
| | | | RNA-binding proteins |
| SN98 | 392604 | FIG. 63 | Sulfatases |
| SN99 | 148696 | FIG. 64 | Nuclear pore complex, Nup98 |
| | | | component (sc |
| | | | Nup145/Nup100/Nup116) |
| SN100 | 395078 | FIG. 63 | Transcription factor containing |
| | | | C2HC type Zn finger |
| SN101 | 417527 | FIG. 64 | GATA-4/5/6 transcription factors |
| SN108 (SEQ | 147679 | FIG. 64 | |
| ID NO: 151) | | | |
| SN109 | 148069 | FIG. 64 | |
| SN110 (SEQ | 150109 | FIG. 64 | |
| ID NO: 157) | | | |
| SN111 (SEQ | 179132 | FIG. 64 | |
| ID NO: 277) | | | |
| SN112 | 184005 | FIG. 64 | |
| SN113 | 282732 | FIG. 64 | Circadian clock protein period |
| SN114 | 293639 | FIG. 64 | |
| SN115 | 294269 | FIG. 64 | Triglyceride lipase-cholesterol |
| | | | esterase |
| SN116 | 298910 | FIG. 64 | |
| SN117 | 306674 | FIG. 64 | FOG: Reverse transcriptase |
| SN118 (SEQ | 311910 | FIG. 64 | |
| ID NO: 283) | | | |
| SN119 | 316556 | FIG. 64 | Transcription factor NERF and |
| | | | related proteins, contain ETS domain |
| SN120 (SEQ | 390379 | FIG. 64 | |
| ID NO: 163) | | | |
| SN121 | 394711 | FIG. 64 | |
| SN122 (SEQ | 413890 | FIG. 64 | |
| ID NO: 289) | | | |
| SN123 | 419587 | FIG. 64 | Oxidoreductase |
| SN124 (SEQ | 183755 | FIG. 63 | |
| ID NO: 169) | | | |
| SN125 | 334004 | FIG. 63 | |
| SN126 | 378057 | FIG. 63 | |
| SN127 | 404363 | FIG. 63 | |
| SN128 (SEQ | 417505 | FIG. 63 | |
| ID NO: 295) | | | |
| SN129 | 154760 | FIG. 63 | |
| SN130 | 311088 | FIG. 63 | |
| SN131 | 311909 | FIG. 63 | |
| SN132 | 379145 | FIG. 63 | |
| SN133 | 406782 | FIG. 63 | |
| SN134 | 147935 | FIG. 63 | |
| SN135 | 177356 | FIG. 63 | |
| SN136 | 301553 | FIG. 63 | |
| SN137 | 322323 | FIG. 63 | |

### Example 8

### Cloning of SN Genes and Creation of Transgenic Lines

Because of the importance of the nitrogen utilization pathways not only in lipid production but also in growth, photosynthesis and productivity, the nitrogen stress pathways have been studied further. Over 100 additional genes were selected based on the nitrogen starvation and SN03 overexpression transcriptomics and each of these genes were engineered as an overexpression cell line in Chlamydomonas, as described above. The vector used for cloning and transformation was nuclear transformation vector Ble2a (as shown in FIG. 34). Additionally, other vectors used were based on the vector of FIG. 34 with the addition of a second selection cassette for paromomycin and the addition of a FLAG-Mat protein tag (FIG. 63 and FIG. 64). Table 7 above lists the vectors that were used for each SN gene. As a result, at least 12 independent transgenic lines for each of the SN genes were created.

### Example 9

### Lipid Phenotype Screening

131 target genes were identified from the nitrogen starvation and SN03 overexpression transcriptomics. Multiple lines for each transgene were screened for changes in lipid content and/or profile. Screening by lipid dyes (Guava Screening Data) and by chemical extraction (Lipid Screening Data) was used to identify an initial set of transgenic lines with potential lipid phenotypes. A more rigorous chemical extraction (Lipid Extraction Data) was conducted with these putative winners.

The genes that impact lipid accumulation, content and/or profile in C. reinhardtii are listed in the Table 8 along with the Joint Genome Institute (JGI) protein ID and functional annotation. Also included in Table 8 are the sequence identification numbers for the genes.

TABLE 8
Lipid Trait Genes.

| | JGI | |
|---|---|---|
| SN | Protein ID | Functional Annotation |
| SN02 (SEQ | 151215 | HMG box-containing protein |
| ID NO: 61) | | |
| SN03 (SEQ | 147817 | CREB binding protein/P300 and related |
| ID NO: 67) | | TAZ Zn-finger proteins |
| SN08 (SEQ | 149064 | HMG-box transcription factor |
| ID NO: 73) | | |
| SN09 (SEQ | 286781 | Nuclear receptor coregulator SMRT/SMRTER, |
| ID NO: 79) | | contains Myb-like domains |
| SN11 (SEQ | 289473 | CREB binding protein/P300 and related |
| ID NO: 85) | | TAZ Zn-finger proteins |
| SN21 (SEQ | 169264 | Xanthine/uracil transporters |
| ID NO: 91) | | |
| SN26 (SEQ | 382107 | hypothetical protein |
| ID NO: 97) | | |
| SN39 (SEQ | 303091 | Predicted membrane protein, contains DoH |
| ID NO: 103) | | and Cytochrome b-561/ferric reductase |
| | | transmembrane domains |
| SN71 (SEQ | 153527 | Nuclear receptor coregulator SMRT/SMRTER, |
| ID NO: 109) | | contains Myb-like domains |
| SN75 (SEQ | 149959 | Transcription factor containing C2HC |
| ID NO: 115) | | type Zn finger |
| SN80 (SEQ | 196335 | Na+/Pi symporter |
| ID NO: 121) | | |
| SN81 (SEQ | 405943 | Predicted E3 ubiquitin ligase |
| ID NO: 127) | | |
| SN84 (SEQ | 151805 | Uncharacterized conserved protein, |
| ID NO: 133) | | contains BTB/POZ domain |
| SN87 (SEQ | 333839 | Defense-related protein containing SCP |
| ID NO: 139) | | domain |
| SN91 (SEQ | 399766 | von Willebrand factor and related |
| ID NO: 145) | | coagulation proteins |
| SN108 (SEQ | 147679 | hypothetical protein |
| ID NO: 151) | | |
| SN110 (SEQ | 150109 | hypothetical protein |
| ID NO: 157) | | |
| SN120 (SEQ | 390379 | hypothetical protein |
| ID NO: 163) | | |
| SN124 (SEQ | 183755 | hypothetical protein |
| ID NO: 169) | | |

A list of the codon-optimized gene sequences (represented by SEQ ID NOs.) that were each cloned into a Ble2A expression construct is provided below in Table 9.

SN02 (SEQ ID NO: 63)
SN03 (SEQ ID NO: 69)
SN08 (SEQ ID NO: 75)
SN09 (SEQ ID NO: 81)
SN11 (SEQ ID NO: 87)
SN21 (SEQ ID NO: 93)
SN26 (SEQ ID NO: 99)
SN39 (SEQ ID NO: 105)
SN71 (SEQ ID NO: 111)
SN75 (SEQ ID NO: 117)
SN80 (SEQ ID NO: 123)
SN81 (SEQ ID NO: 129)
SN84 (SEQ ID NO: 135)
SN87 (SEQ ID NO: 141)
SN91 (SEQ ID NO: 147)
SN108 (SEQ ID NO: 153)
SN110 (SEQ ID NO: 159)
SN120 (SEQ ID NO: 165)
SN124 (SEQ ID NO: 171)

### Example 10

### Microextraction-Lipid Screening Data

All lines were screened using a quick microextraction method. Cultures were grown in 96 well blocks and were pelleted by centrifugation. Each 8×12 block represents a series of 12 transgenic lines of 8 individual SN genes. The pelleted biomass was extracted by sonicating in a solvent mixture consisting of acetonitrile (35%), methanol (26%), tetrahydrofuran (9%) and methyl-tert-butyl ether (30%). The extraction mixture was centrifuged and the supernatant was analyzed by HPLC using ELSD to screen for changes in lipid accumulation and chlorophyll production relative to a wild-type control.

Shown below are the data for candidate winners. Classes of molecules were binned for analysis, with the values in the tables representing summed area under the curve on the HPLC chromatogram. Rows represent individual transgenic lines. Any increase in a molecule class is underlined, starting at 2× the average value over the entire plate containing 96 strains representing up to 8 SN genes (listed on the first line of each set as “Pool avg”). The classes of molecules represented in the columns are: Heme (chlorophylides and related polar breakdown products), Polar (Polar lipids), Chlor b (Chlorophyll b). Chlor a (Chlorophyll a), Pheophytin and TAG (triacylglycerol, including diacylglycerols as well).

Gene Mix #1

| Sample | Heme | Polar | Chlor b | Chlor a | Pheophytin | TAG |
|---|---|---|---|---|---|---|
| Pool Avg. | 3.319 | 3.821 | 2.439 | 0.013 | 0.059 | 0.007 |
| SN26.1 | 3.690 | 7.210 | 2.901 | 0.017 | 0.139 | 0.000 |
| SN26.2 | 2.895 | 6.409 | 3.198 | 0.000 | 0.147 | 0.015 |
| SN26.3 | 6.839 | 4.283 | 1.890 | 0.000 | 0.038 | 0.000 |
| SN26.4 | 1.087 | 2.376 | 1.712 | 0.006 | 0.063 | 0.004 |
| SN26.5 | 6.797 | 2.829 | 0.754 | 0.000 | 0.007 | 0.000 |
| SN26.6 | 25.662 | 0.752 | 0.138 | 0.000 | 0.000 | 0.000 |
| SN26.7 | 3.707 | 5.691 | 5.431 | 0.017 | 0.055 | 0.000 |
| SN26.8 | 3.291 | 4.006 | 4.110 | 0.004 | 0.047 | 0.000 |
| SN26.9 | 4.646 | 4.674 | 4.063 | 0.007 | 0.021 | 0.000 |
| SN26.10 | 5.607 | 4.878 | 3.740 | 0.003 | 0.020 | 0.000 |
| SN26.11 | 7.210 | 4.864 | 5.263 | 0.018 | 0.067 | 0.007 |
| SN26.12 | 3.534 | 7.320 | 8.287 | 0.020 | 0.250 | 0.014 |
| SN71.1 | 1.788 | 3.947 | 1.699 | 0.000 | 0.084 | 0.018 |
| SN71.2 | 1.405 | 2.828 | 1.282 | 0.000 | 0.073 | 0.018 |
| SN71.3 | 1.181 | 2.331 | 0.859 | 0.000 | 0.038 | 0.000 |
| SN71.4 | 0.762 | 1.741 | 1.349 | 0.000 | 0.058 | 0.000 |
| SN71.5 | 1.003 | 2.127 | 1.412 | 0.000 | 0.028 | 0.002 |
| SN71.6 | 1.446 | 3.037 | 1.064 | 0.000 | 0.119 | 0.053 |
| SN71.7 | 2.013 | 4.366 | 1.799 | 0.000 | 0.046 | 0.015 |
| SN71.8 | 1.929 | 3.931 | 1.656 | 0.000 | 0.090 | 0.002 |
| SN71.9 | 2.094 | 3.961 | 1.350 | 0.000 | 0.102 | 0.038 |
| SN71.10 | 1.735 | 3.848 | 1.160 | 0.000 | 0.129 | 0.000 |
| SN71.11 | 2.363 | 4.841 | 1.464 | 0.000 | 0.104 | 0.000 |
| SN71.12 | 2.360 | 5.930 | 2.781 | 0.000 | 0.117 | 0.000 |
| SN75.1 | 3.020 | 6.308 | 2.458 | 0.000 | 0.032 | 0.018 |
| SN75.2 | 2.306 | 4.835 | 1.469 | 0.000 | 0.135 | 0.005 |
| SN75.3 | 2.211 | 3.934 | 2.147 | 0.000 | 0.044 | 0.007 |
| SN75.4 | 1.091 | 3.100 | 1.964 | 0.000 | 0.080 | 0.000 |
| SN75.5 | 1.319 | 2.555 | 1.641 | 0.000 | 0.065 | 0.014 |
| SN75.6 | 1.977 | 4.034 | 1.789 | 0.000 | 0.083 | 0.014 |
| SN75.7 | 2.536 | 4.954 | 1.335 | 0.040 | 0.165 | 0.021 |
| SN75.8 | 2.442 | 5.158 | 2.840 | 0.128 | 0.000 | 0.013 |
| SN75.9 | 2.558 | 4.852 | 2.349 | 0.074 | 0.043 | 0.004 |
| SN75.10 | 2.108 | 1.402 | 1.700 | 0.119 | 0.073 | 0.008 |
| SN75.11 | 2.428 | 4.401 | 2.047 | 0.164 | 0.097 | 0.004 |
| SN75.12 | 2.533 | 5.835 | 2.012 | 0.000 | 0.115 | 0.019 |

Gene Mix #2

| Sample | Heme | Polar | Chlor b | Chlor a | Pheophytin | TAG |
|---|---|---|---|---|---|---|
| Pool Avg. | 1.595 | 1.844 | 0.932 | 1.270 | 0.142 | 0.016 |
| SN02.1 | 0.244 | 0.915 | 0.681 | 0.981 | 0.168 | 0.105 |
| SN02.2 | 0.198 | 0.348 | 0.441 | 0.806 | 0.103 | 0.064 |
| SN02.3 | 0.701 | 0.924 | 1.147 | 1.659 | 0.606 | 0.000 |
| SN02.4 | 1.143 | 1.274 | 0.988 | 1.212 | 0.249 | 0.122 |
| SN02.5 | 2.023 | 1.811 | 0.658 | 0.661 | 0.237 | 0.096 |
| SN02.6 | 0.918 | 0.271 | 0.444 | 0.588 | 0.143 | 0.089 |
| SN02.7 | 0.402 | 0.742 | 0.512 | 0.783 | 0.113 | 0.048 |
| SN02.8 | 1.150 | 1.363 | 1.059 | 1.298 | 0.370 | 0.112 |
| SN02.9 | 0.590 | 1.104 | 0.818 | 0.977 | 0.130 | 0.007 |
| SN02.10 | 0.590 | 1.771 | 0.964 | 1.536 | 0.204 | 0.124 |
| SN02.11 | 0.362 | 0.589 | 0.512 | 1.059 | 0.119 | 0.081 |
| SN02.12 | 1.574 | 1.377 | 0.256 | 0.396 | 0.052 | 0.037 |
| SN21.1 | 0.858 | 1.185 | 1.076 | 1.441 | 0.363 | 0.089 |
| SN21.2 | 0.669 | 1.121 | 0.963 | 1.420 | 0.330 | 0.104 |
| SN21.3 | 0.392 | 0.678 | 0.619 | 0.978 | 0.152 | 0.033 |
| SN21.4 | 1.370 | 1.974 | 1.317 | 1.765 | 0.457 | 0.131 |
| SN21.5 | 1.093 | 1.768 | 1.034 | 1.438 | 0.252 | 0.107 |
| SN21.6 | 1.940 | 1.074 | 0.416 | 0.345 | 0.106 | 0.031 |
| SN21.7 | 1.071 | 0.585 | 0.906 | 1.273 | 0.326 | 0.202 |
| SN21.8 | 1.543 | 1.810 | 1.443 | 1.628 | 0.511 | 0.220 |
| SN21.9 | 0.681 | 0.185 | 0.415 | 0.597 | 0.128 | 0.070 |
| SN21.10 | 0.280 | 0.370 | 0.440 | 0.809 | 0.125 | 0.049 |
| SN21.11 | 0.702 | 0.957 | 0.855 | 1.270 | 0.313 | 0.112 |
| SN21.12 | 1.270 | 2.226 | 1.296 | 1.520 | 0.458 | 0.168 |

Gene Mix #7

| Sample | Heme | Polar | Chlor b | Chlor a | Pheophytin | TAG |
|---|---|---|---|---|---|---|
| Pool Avg. | 3.792 | 4.841 | 2.624 | 2.678 | 0.247 | 0.067 |
| SN39.1 | 4.825 | 7.806 | 4.832 | 4.415 | 0.664 | 0.049 |
| SN39.2 | 5.348 | 7.787 | 4.253 | 4.477 | 0.361 | 0.184 |
| SN39.3 | 4.775 | 4.235 | 2.776 | 3.099 | 0.123 | 0.019 |
| SN39.4 | 4.747 | 10.428 | 0.000 | 4.927 | 0.586 | 0.074 |
| SN39.5 | 4.297 | 4.292 | 1.697 | 2.314 | 0.157 | 0.006 |
| SN39.6 | 4.643 | 5.641 | 2.764 | 2.804 | 0.164 | 0.016 |
| SN39.7 | 4.466 | 3.973 | 1.763 | 1.637 | 0.083 | 0.012 |
| SN39.8 | 5.085 | 3.792 | 1.808 | 1.517 | 0.038 | 0.006 |
| SN39.9 | 3.817 | 6.120 | 4.186 | 3.471 | 0.375 | 0.126 |
| SN39.10 | 4.315 | 6.563 | 3.744 | 3.687 | 0.330 | 0.085 |
| SN39.11 | 3.761 | 5.564 | 3.227 | 2.993 | 0.202 | 0.025 |
| SN39.12 | 10.702 | 0.000 | 0.000 | 0.263 | 0.263 | 0.000 |
| SN80.1 | 3.884 | 5.683 | 2.627 | 2.883 | 0.343 | 0.205 |
| SN80.2 | 4.185 | 5.974 | 3.959 | 3.821 | 0.375 | 0.094 |
| SN80.3 | 3.194 | 5.808 | 2.452 | 2.658 | 0.399 | 0.147 |
| SN80.4 | 3.766 | 4.095 | 1.837 | 2.201 | 0.255 | 0.035 |
| SN80.5 | 4.025 | 4.562 | 2.522 | 2.520 | 0.130 | 0.021 |
| SN80.6 | 3.311 | 4.681 | 2.593 | 2.874 | 0.298 | 0.116 |
| SN80.7 | 3.347 | 4.785 | 2.437 | 2.693 | 0.372 | 0.112 |
| SN80.8 | 3.284 | 5.248 | 3.420 | 3.364 | 0.413 | 0.174 |
| SN80.9 | 3.945 | 6.264 | 3.345 | 3.664 | 0.286 | 0.041 |
| SN80.10 | 2.469 | 3.986 | 2.362 | 1.966 | 0.178 | 0.025 |
| SN80.11 | 3.950 | 2.396 | 1.553 | 0.417 | 0.009 | 0.000 |
| SN80.12 | 4.024 | 3.922 | 1.495 | 1.126 | 0.060 | 0.003 |
| SN81.1 | 3.770 | 1.835 | 0.727 | 0.096 | 0.002 | 0.002 |
| SN81.2 | 4.529 | 4.337 | 2.310 | 0.979 | 0.134 | 0.000 |
| SN81.3 | 3.636 | 4.620 | 2.368 | 2.508 | 0.246 | 0.031 |
| SN81.4 | 4.452 | 5.886 | 2.860 | 2.891 | 0.192 | 0.109 |
| SN81.5 | 4.723 | 6.974 | 4.556 | 4.781 | 0.545 | 0.596 |
| SN81.6 | 2.901 | 4.151 | 2.230 | 2.826 | 0.264 | 0.018 |
| SN81.7 | 2.826 | 3.912 | 2.522 | 2.692 | 0.147 | 0.030 |
| SN81.8 | 3.287 | 5.108 | 3.097 | 3.122 | 0.411 | 0.083 |
| SN81.9 | 3.029 | 4.251 | 2.133 | 2.206 | 0.270 | 0.152 |
| SN81.10 | 3.624 | 5.011 | 3.125 | 3.272 | 0.238 | 0.060 |
| SN81.11 | 2.780 | 3.765 | 3.192 | 2.434 | 0.268 | 0.030 |
| SN81.12 | 2.806 | 3.200 | 1.760 | 1.554 | 0.265 | 0.025 |

Gene Mix #9

| Sample | Heme | Polar | Chlor b | Chlor a | Pheophytin | TAG |
|---|---|---|---|---|---|---|
| Pool Avg. | 3.784 | 2.166 | 1.776 | 2.488 | 0.272 | 0.008 |
| SN08-1 | 2.455 | 2.088 | 1.606 | 2.377 | 0.181 | 0.000 |
| SN08-2 | 3.042 | 1.566 | 1.709 | 2.492 | 0.354 | 0.000 |
| SN08-3 | 3.162 | 1.560 | 2.037 | 2.495 | 0.352 | 0.000 |
| SN08-4 | 3.301 | 0.221 | 0.681 | 0.624 | 0.038 | 0.000 |
| SN08-5 | 2.607 | 1.868 | 2.466 | 3.505 | 0.451 | 0.011 |
| SN08-6 | 1.528 | 0.448 | 0.977 | 1.595 | 0.090 | 0.000 |
| SN08-7 | 2.277 | 0.490 | 0.912 | 1.417 | 0.126 | 0.000 |
| SN08-8 | 2.419 | 0.248 | 0.688 | 0.941 | 0.091 | 0.000 |
| SN08-9 | 3.239 | 1.411 | 1.161 | 2.122 | 0.339 | 0.000 |
| SN08-10 | 3.317 | 2.158 | 2.252 | 3.005 | 0.332 | 0.015 |
| SN08-11 | 2.563 | 1.680 | 2.058 | 3.174 | 0.558 | 0.013 |
| SN08-12 | 1.464 | 0.227 | 1.251 | 2.353 | 0.314 | 0.000 |
| SN09-1 | 6.896 | 2.145 | 1.327 | 2.080 | 0.231 | 0.000 |
| SN09-2 | 2.736 | 1.665 | 1.558 | 2.061 | 0.182 | 0.005 |
| SN09-3 | 1.190 | 0.190 | 0.521 | 0.908 | 0.086 | 0.000 |
| SN09-4 | 1.884 | 0.523 | 0.763 | 1.286 | 0.160 | 0.000 |
| SN09-5 | 1.985 | 1.897 | 1.951 | 2.778 | 0.453 | 0.000 |
| SN09-6 | 2.771 | 1.595 | 0.000 | 0.000 | 0.000 | 0.000 |
| SN09-6 | 1.778 | 2.764 | 3.032 | 0.000 | 0.658 | 0.000 |
| SN09-7 | 2.504 | 0.626 | 0.964 | 0.988 | 0.272 | 0.000 |
| SN09-8 | 1.485 | 2.164 | 2.125 | 2.457 | 0.458 | 0.000 |
| SN09-9 | 1.708 | 2.117 | 1.942 | 2.398 | 0.363 | 0.000 |
| SN09-10 | 1.890 | 2.030 | 1.808 | 1.646 | 0.280 | 0.000 |
| SN09-11 | 18.052 | 2.876 | 1.495 | 0.378 | 0.057 | 0.000 |
| SN09-12 | 3.671 | 3.957 | 3.279 | 3.605 | 1.140 | 0.000 |
| SN87-1 | 9.955 | 3.795 | 2.607 | 3.486 | 0.252 | 0.010 |
| SN87-2 | 0.876 | 0.000 | 0.000 | 0.000 | 0.000 | 0.000 |
| SN87-3 | 3.075 | 3.874 | 3.035 | 4.399 | 0.447 | 0.009 |
| SN87-4 | 7.170 | 0.446 | 1.125 | 1.393 | 0.036 | 0.000 |
| SN87-5 | 5.386 | 5.498 | 3.864 | 5.486 | 0.464 | 0.019 |
| SN87-6 | 5.445 | 3.567 | 2.882 | 4.436 | 0.235 | 0.024 |
| SN87-7 | 3.513 | 1.449 | 1.678 | 2.014 | 0.102 | 0.004 |
| SN87-8 | 4.734 | 4.793 | 2.935 | 4.426 | 0.338 | 0.015 |
| SN87-9 | 4.203 | 5.097 | 3.170 | 5.184 | 0.546 | 0.015 |
| SN87-10 | 2.460 | 2.770 | 2.244 | 3.097 | 0.358 | 0.017 |
| SN87-11 | 6.682 | 1.403 | 1.294 | 2.254 | 0.164 | 0.010 |
| SN87-12 | 3.839 | 0.297 | 0.362 | 0.601 | 0.033 | 0.016 |
| SN91-1 | 19.524 | 1.885 | 1.941 | 2.691 | 0.214 | 0.017 |
| SN91-2 | 3.246 | 0.594 | 1.314 | 1.897 | 0.131 | 0.000 |
| SN91-3 | 4.680 | 3.879 | 3.776 | 4.550 | 0.738 | 0.025 |
| SN91-4 | 2.703 | 2.151 | 1.721 | 2.500 | 0.277 | 0.012 |
| SN91-5 | 3.691 | 3.570 | 2.779 | 3.808 | 0.296 | 0.018 |
| SN91-6 | 2.741 | 2.517 | 2.054 | 2.794 | 0.531 | 0.015 |
| SN91-7 | 4.950 | 1.391 | 1.266 | 2.034 | 0.146 | 0.013 |
| SN91-8 | 4.644 | 3.338 | 2.575 | 3.455 | 0.435 | 0.022 |
| SN91-9 | 2.690 | 2.986 | 2.426 | 3.374 | 0.502 | 0.021 |
| SN91-10 | 1.908 | 1.728 | 1.697 | 2.425 | 0.345 | 0.013 |
| SN91-11 | 4.391 | 3.446 | 2.716 | 3.938 | 0.528 | 0.021 |
| SN91-12 | 3.157 | 3.684 | 3.130 | 4.037 | 0.950 | 0.029 |

Gene Mix #10

| Sample | Heme | Polar | Chlor b | Chlor a | Pheophytin | TAG |
|---|---|---|---|---|---|---|
| Pool Avg. | 7.180 | 1.361 | 1.985 | 2.502 | 0.480 | 0.023 |
| SN11-1 | 7.111 | 0.913 | 2.180 | 2.764 | 0.350 | 0.000 |
| SN11-2 | 13.400 | 1.286 | 1.495 | 1.935 | 0.151 | 0.000 |
| SN11-3 | 9.900 | 1.448 | 1.632 | 2.469 | 0.341 | 0.006 |
| SN11-4 | 57.685 | 0.000 | 0.000 | 0.000 | 0.000 | 0.000 |
| SN11-5 | 3.632 | 2.313 | 2.172 | 2.896 | 0.579 | 0.014 |
| SN11-6 | 6.534 | 2.249 | 2.141 | 2.885 | 0.564 | 0.026 |
| SN11-7 | 7.514 | 1.907 | 2.083 | 2.762 | 0.359 | 0.018 |
| SN11-8 | 6.139 | 0.996 | 1.121 | 1.758 | 0.377 | 0.027 |
| SN11-9 | 6.958 | 1.855 | 1.834 | 2.701 | 0.519 | 0.011 |
| SN11-10 | 6.710 | 1.694 | 1.652 | 2.141 | 0.449 | 0.007 |
| SN11-11 | 5.553 | 1.321 | 1.728 | 2.434 | 0.522 | 0.025 |
| SN11-12 | 11.832 | 0.094 | 0.272 | 0.581 | 0.065 | 0.000 |
| SN84-1 | 10.585 | 0.109 | 1.200 | 1.842 | 0.194 | 0.014 |
| SN84-2 | 18.751 | 5.455 | 5.612 | 6.010 | 1.161 | 0.032 |
| SN84-3 | 12.374 | 4.939 | 5.513 | 6.133 | 1.365 | 0.081 |
| SN84-4 | 8.568 | 2.835 | 4.747 | 5.264 | 1.096 | 0.046 |
| SN84-5 | 13.382 | 0.800 | 2.785 | 3.814 | 0.659 | 0.055 |
| SN84-6 | 14.271 | 10.090 | 8.090 | 9.942 | 2.297 | 0.124 |
| SN84-7 | 6.811 | 1.596 | 2.933 | 4.135 | 0.720 | 0.035 |
| SN84-8 | 6.974 | 0.309 | 1.587 | 1.952 | 0.213 | 0.009 |
| SN84-9 | 6.949 | 2.773 | 4.990 | 5.785 | 1.156 | 0.031 |
| SN84-10 | 9.680 | 2.535 | 4.705 | 5.508 | 0.896 | 0.045 |
| SN84-11 | 10.477 | 0.228 | 2.861 | 3.234 | 0.340 | 0.033 |
| SN84-12 | 10.240 | 0.993 | 3.461 | 3.751 | 0.537 | 0.033 |

Gene Mix #11

| Sample | Heme | Polar | Chlor b | Chlor a | Pheophytin | TAG |
|---|---|---|---|---|---|---|
| Pool Avg. | 5.235 | 0.324 | 1.331 | 1.456 | 0.349 | 0.030 |
| SN108-1 | 3.869 | 0.144 | 0.925 | 0.914 | 0.376 | 0.038 |
| SN108-2 | 6.517 | 1.369 | 3.393 | 3.103 | 1.123 | 0.101 |
| SN108-3 | 8.186 | 0.590 | 2.683 | 2.588 | 0.801 | 0.070 |
| SN108-4 | 6.771 | 0.076 | 1.225 | 1.129 | 0.304 | 0.043 |
| SN108-5 | 5.406 | 1.092 | 2.600 | 2.672 | 0.859 | 0.019 |
| SN108-6 | 6.298 | 0.821 | 2.266 | 2.488 | 0.858 | 0.096 |
| SN108-7 | 6.428 | 0.264 | 1.670 | 1.662 | 0.362 | 0.048 |
| SN108-8 | 3.854 | 0.277 | 1.481 | 1.565 | 0.389 | 0.023 |
| SN108-9 | 5.169 | 0.625 | 2.150 | 2.392 | 0.636 | 0.063 |
| SN108-10 | 8.021 | 0.758 | 2.950 | 3.187 | 0.942 | 0.100 |
| SN108-11 | 8.851 | 0.622 | 2.671 | 2.666 | 0.783 | 0.068 |
| SN108-12 | 9.666 | 1.062 | 3.088 | 3.134 | 0.826 | 0.079 |
| SN110-1 | 6.759 | 0.265 | 1.986 | 1.951 | 0.421 | 0.048 |
| SN110-2 | 3.989 | 0.078 | 1.342 | 1.114 | 0.285 | 0.031 |
| SN110-3 | 3.406 | 0.040 | 0.707 | 0.797 | 0.233 | 0.034 |
| SN110-4 | 14.932 | 0.082 | 0.171 | 0.029 | 0.012 | 0.000 |
| SN110-5 | 0.000 | 0.000 | 0.000 | 0.000 | 0.000 | 0.000 |
| SN110-6 | 6.672 | 0.140 | 1.280 | 1.855 | 0.365 | 0.032 |
| SN110-7 | 3.022 | 0.000 | 0.359 | 0.302 | 0.101 | 0.019 |
| SN110-8 | 15.469 | 0.197 | 0.489 | 0.799 | 0.142 | 0.006 |
| SN110-9 | 11.941 | 0.552 | 1.090 | 1.531 | 0.260 | 0.005 |
| SN110-10 | 14.271 | 0.305 | 0.517 | 0.842 | 0.136 | 0.006 |
| SN110-11 | 22.520 | 0.064 | 0.070 | 0.020 | 0.009 | 0.000 |
| SN110-12 | 5.877 | 0.968 | 2.264 | 2.455 | 0.788 | 0.065 |
| SN120-1 | 5.649 | 0.267 | 1.721 | 1.556 | 0.397 | 0.028 |
| SN120-2 | 5.340 | 0.195 | 1.113 | 1.280 | 0.245 | 0.029 |
| SN120-3 | 3.429 | 0.029 | 0.602 | 0.639 | 0.128 | 0.015 |
| SN120-4 | 4.739 | 0.082 | 1.312 | 1.065 | 0.266 | 0.027 |
| SN120-5 | 3.868 | 0.083 | 1.099 | 0.982 | 0.250 | 0.016 |
| SN120-6 | 4.122 | 0.060 | 0.903 | 0.813 | 0.379 | 0.012 |
| SN120-7 | 3.265 | 0.155 | 1.271 | 1.251 | 0.253 | 0.034 |
| SN120-8 | 4.209 | 0.119 | 1.116 | 1.132 | 0.234 | 0.019 |
| SN120-9 | 4.267 | 0.183 | 1.279 | 1.246 | 0.283 | 0.027 |
| SN120-10 | 6.206 | 0.225 | 1.240 | 1.277 | 0.287 | 0.026 |
| SN120-11 | 2.416 | 0.013 | 0.528 | 0.609 | 0.124 | 0.021 |
| SN120-12 | 5.449 | 0.014 | 0.972 | 0.736 | 0.156 | 0.014 |

Gene Mix #12

| Sample | Heme | Polar | Chlor b | Chlor a | Pheophytin | TAG |
|---|---|---|---|---|---|---|
| Pool Avg. | 6.159 | 1.051 | 1.828 | 2.790 | 0.388 | 0.027 |
| SN124-1 | 6.160 | 1.200 | 1.938 | 3.021 | 0.489 | 0.040 |
| SN124-2 | 5.355 | 0.843 | 0.070 | 2.241 | 0.322 | 0.023 |
| SN124-3 | 7.056 | 1.314 | 2.665 | 3.962 | 0.531 | 0.044 |
| SN124-4 | 8.573 | 1.732 | 2.596 | 3.978 | 0.586 | 0.046 |
| SN124-5 | 8.476 | 2.244 | 2.820 | 4.536 | 0.651 | 0.049 |
| SN124-6 | 8.201 | 2.438 | 3.430 | 4.664 | 0.735 | 0.053 |
| SN124-7 | 6.637 | 1.331 | 3.053 | 3.896 | 0.591 | 0.040 |
| SN124-8 | 8.936 | 5.405 | 4.530 | 6.311 | 0.642 | 0.052 |
| SN124-9 | 5.927 | 1.604 | 2.269 | 3.535 | 0.541 | 0.041 |
| SN124-10 | 8.693 | 0.738 | 2.045 | 3.107 | 0.410 | 0.033 |
| SN124-11 | 10.107 | 0.750 | 1.858 | 2.936 | 0.433 | 0.032 |
| SN124-12 | 6.085 | 1.841 | 2.837 | 3.601 | 0.780 | 0.042 |

### Example 11

### Guava Screening Data

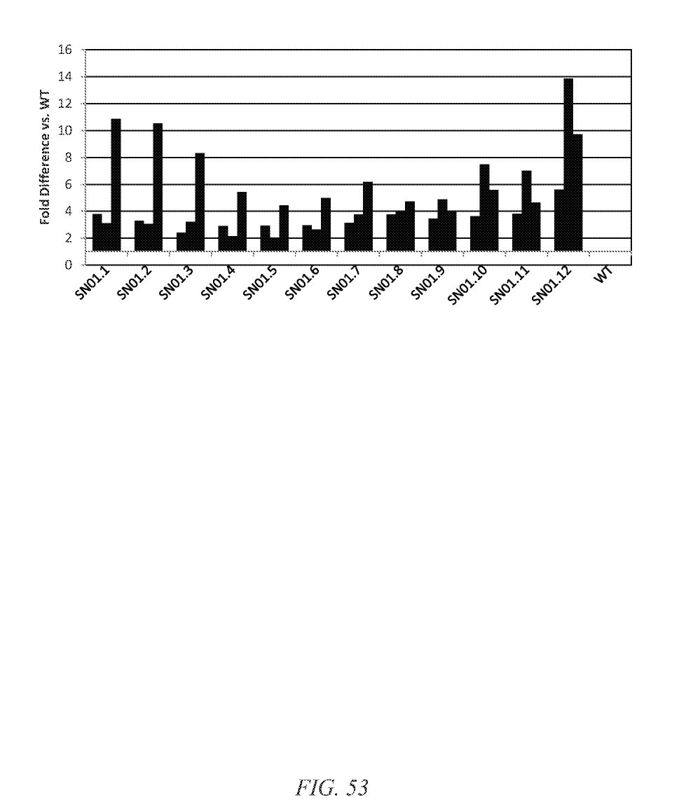

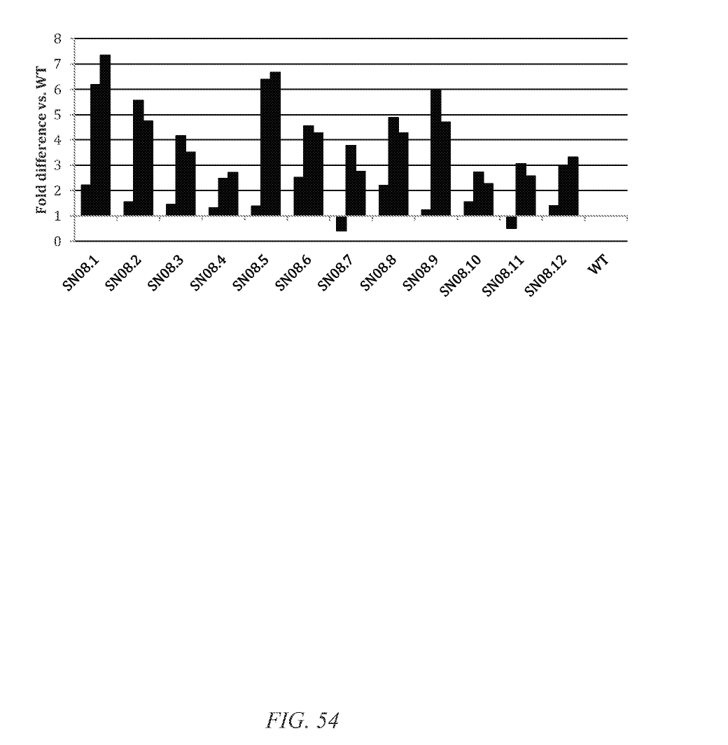

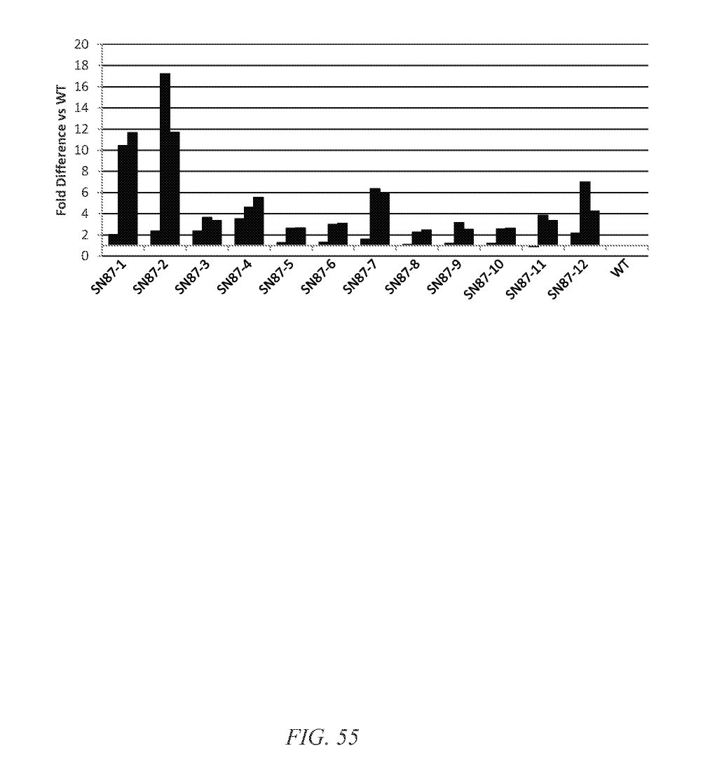

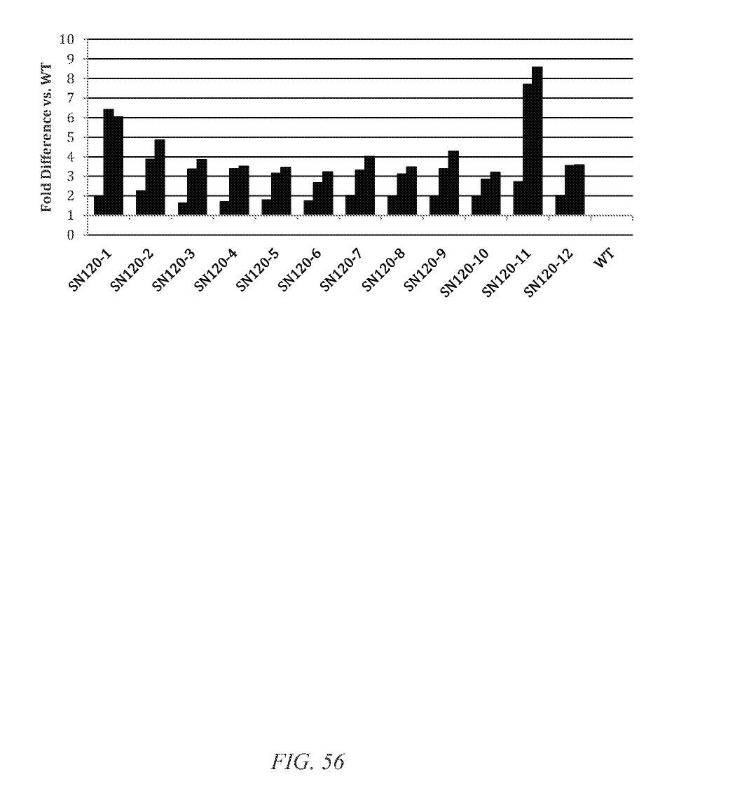

A lipid dye-based assay was also used to screen the SN gene lines for lipid content. Analytical flow cytometry (Guava) is a direct measurement of fluorescence used when cultures are stained separately with three lipid dyes: Bodipy, Nile Red and LipidTOX Green. All three dyes are lipophilic, with specific, but ill-defined, affinities for different lipid components in the cell. Use of three different dyes gives a wider range of possible lipid phenotypes that can be observed. Of interest are genes that change the overall amount of lipid, but also in those that modify the lipid profile by affecting a subset of lipids. Each individual line was measured and compared to a wild-type C. reinhardii sample. Winners were determined based on their performance relative to the wild-type control in the Guava screen. Representative data is shown in FIG. 53. FIG. 54, FIG. 55, and FIG. 56.

### Example 12

### Lipid Extraction Data

Potential winners from the Guava Screening Data and quick microextractions (Lipid Screening Data) were selected for an additional extraction-based assay. Of the transgenic lines selected after the two screens, 20 were selected for a more in-depth analysis using a small-scale extraction in conjunction with LC-MS/MS to identify major lipids as well as chlorophyll and its breakdown products. Approximately 1 L of culture was grown and harvested biomass was dried and extracted by sonicating in a solvent mixture consisting of acetonitrile (35%), methanol (26%), tetrahydrofuran (9%) and methyl-tert-butyl ether (30%). The lipid yields were determined gravimetrically after evaporation of solvent under a stream of nitrogen. The extracted oils were analyzed by HPLC-MS/MS for changes in lipid production relative to the wild-type control.

In comparing the wild-type control to a nitrogen starved wild-type sample, it can readily be seen that triacylglycerols (TAG's) increase significantly, whereas both chlorophyll a and chlorophyll b production are decreased as expected. Two of the lines with the highest TAG's (more than 2-fold that of wild type), SN120 and SN91 both have decreased levels of chlorophyll a and b which is consistent with a nitrogen starved phenotype. In addition, SN91, SN120, SN03 and the nitrogen starved wild type control all exhibit decreased levels of DGDG (digalactosyl diacylglycerol).

Of the SN genes analyzed by LC-MS/MS, several show a significant increase in the production of diacylglyceryl trimethylhomoserine (DGTS) a membrane lipid which is used in place of phospholipids when phosphate levels are limited. Lines exhibiting increased levels of DGTS in a 2-fold or more excess of the wild type control include: SN08, SN75 and SN108. These lines also had an increase in extractable material versus the wild type control.

Several of the lines with the highest extractables including SN28 and SN124, show a decrease in the level of chlorophyll a with no apparent change in the accumulation of lipids analyzed in this study.

Data is presented below in Table 10 and Table 11 for the twenty genes and wild type controls (nitrogen starved and nitrogen replete). Total gravimetric lipid yield is listed in the first row (% Yield) with the component molecules of this extracted oil listed with their respective percent of the total yield. Some minor components are not listed so totals do not equal 100%.

TABLE 10

| Type | SN02 | SN08 | SN09 | SN11 | SN21 | SN26 |
|---|---|---|---|---|---|---|
| % Yield | 25.98 | 27.46 | 26.09 | 27.39 | 25.13 | 26.17 |
| Carotene | 0.7 | 0.3 | 0.6 | 0.6 | 0.3 | 0.7 |
| Chlorophyll a | 12.0 | 10.8 | 8.3 | — | 7.9 | 8.3 |
| Chlorophyll b | — | 3.1 | 0.8 | — | 2.3 | 3.7 |
| DAG | 17.6 | 7.3 | 14.0 | 14.9 | 5.4 | 17.3 |
| DGDG | 4.8 | 1.0 | 4.1 | 3.9 | 1.0 | 3.4 |
| DGTS | 10.7 | 20.2 | 9.4 | 16.8 | 17.4 | 10.0 |
| LPC | 0.3 | 1.0 | 0.9 | 0.6 | — | 0.3 |
| MGDG | 3.1 | 6.9 | 2.9 | 2.5 | 8.6 | — |
| MAG | — | 0.7 | — | — | 1.1 | — |
| PG | — | — | — | 0.1 | — | — |
| Pheophytin a | 12.9 | 10.2 | 13.2 | 15.5 | 4.7 | 21.0 |
| Pheophytin b | — | — | — | — | — | 0.1 |
| TAG | 1.4 | 2.9 | 4.7 | 1.3 | 6.4 | 4.4 |
| Unknown | 25.7 | 30.3 | 24.4 | 29.4 | 38.9 | 21.1 |

| Type | SN28 | SN39 | SN71 | SN75 | WT-Nit | WT |
|---|---|---|---|---|---|---|
| % Yield | 33.17 | 30.25 | 26.99 | 30.17 | 25.90 | 26.67 |
| Carotene | 0.7 | 0.7 | 0.7 | 0.3 | 0.3 | 0.9 |
| Chlorophyll a | — | 12.4 | 7.3 | 9.8 | 1.4 | 6.1 |
| Chlorophyll b | 2.9 | 3.8 | 3.8 | 3.3 | 0.4 | 5.3 |
| DAG | 19.6 | 14.0 | 8.0 | 6.3 | 3.4 | 15.2 |
| DGDG | 4.7 | 4.6 | 6.5 | 1.2 | 0.6 | 7.0 |
| DGTS | 9.7 | 6.9 | 9.6 | 23.1 | 11.7 | 6.9 |
| LPC | — | 0.4 | 1.1 | 1.3 | 0.2 | — |
| MGDG | 2.4 | — | 2.5 | 7.6 | 6.5 | — |
| MAG | — | — | — | 1.0 | 0.7 | 2.5 |
| PG | — | — | — | — | — | — |
| Pheophytin a | 8.9 | 12.8 | 11.1 | 8.3 | 10.8 | 11.3 |
| Pheophytin b | — | — | — | — | — | — |
| TAG | 1.5 | 1.1 | 11.4 | 3.6 | 43.6 | 4.4 |
| unknown | 32.0 | 28.4 | 22.8 | 29.4 | 18.1 | 27.5 |

Key: DAG (diacylglycerols); DGDG (digalactosyl diacylglycerol); DGTS (Diacylglyceryl trimethylhomoserine); LPC (lysophosphatidylcholine); MGDG (monogalactosyl diacylglycerol); MAG (monoacylglycerols); PG (Phosphatidylglycerols); and TAG (triacylglycerols).

TABLE 11

| Type | SN80 | SN81 | SN84 | SN87 | SN91 | SN108 |
|---|---|---|---|---|---|---|
| % Yield | 26.60 | 32.81 | 25.94 | 24.57 | 28.85 | 27.33 |
| Carotene | 0.7 | 0.5 | 0.6 | 0.8 | 0.6 | 0.3 |
| Chlorophyll a | 5.5 | 6.3 | 10.6 | 1.6 | 3.1 | 11.1 |
| Chlorophyll b | — | 0.4 | 3.1 | 1.9 | 2.1 | 2.8 |
| DAG | 20.4 | 11.1 | 20.3 | 22.1 | 13.1 | 5.0 |
| DGDG | 3.8 | 5.5 | 3.8 | 1.4 | 2.0 | 1.1 |
| DGTS | 5.6 | 4.4 | 5.9 | 16.8 | 5.3 | 23.9 |
| LPC | 0.9 | 0.2 | 0.3 | 0.4 | 1.0 | 0.5 |
| MGDG | — | 1.6 | 1.9 | 1.7 | 1.7 | 11.6 |
| MAG | 0.9 | — | 0.3 | — | — | 1.1 |
| PG | — | — | — | — | — | — |
| Pheophytin a | 12.0 | 27.4 | 10.5 | — | 13.1 | 6.0 |
| Pheophytin b | — | — | — | — | — | — |
| TAG | 1.9 | 2.3 | 1.6 | 3.8 | 10.2 | 4.7 |
| Unknown | 32.9 | 30.3 | 22.1 | 31.2 | 32.4 | 26.1 |

| Type | SN110 | SN120 | SN124 | SN03 | WT-Nit | WT |
|---|---|---|---|---|---|---|
| % Yield | 21.74 | 23.10 | 35.63 | 35.72 | 25.90 | 26.67 |
| Carotene | 0.8 | 0.3 | 0.7 | 0.6 | 0.3 | 0.9 |
| Chlorophyll a | 6.0 | 2.5 | — | 5.4 | 1.4 | 6.1 |
| Chlorophyll b | 2.0 | 0.9 | 5.6 | 3.3 | 0.4 | 5.3 |
| DAG | 13.8 | — | 16.0 | 8.1 | 3.4 | 15.2 |
| DGDG | 6.2 | 0.1 | 3.5 | 1.0 | 0.6 | 7.0 |
| DGTS | 14.9 | 15.7 | 0.4 | 11.4 | 11.7 | 6.9 |
| LPC | — | 1.4 | 0.4 | 0.4 | 0.2 | — |
| MGDG | 0.8 | 5.9 | 4.1 | 2.2 | 6.5 | — |
| MAG | — | — | — | — | 0.7 | 2.5 |
| PG | — | — | — | — | — | — |
| Pheophytin a | 13.7 | 19.5 | 18.6 | 15.9 | 10.8 | 11.3 |
| Pheophytin b | — | — | — | — | — | — |
| TAG | 2.6 | 10.7 | 2.1 | 2.6 | 43.6 | 4.4 |
| unknown | 24.8 | 31.6 | 33.1 | 27.9 | 18.1 | 27.5 |

Key: DAG (diacylglycerols); DGDG (digalactosyl diacylglycerol); DGTS (Diacylglyceryl trimethylhomoserine); LPC (lysophosphatidylcholine); MGDG (monogalactosyl diacylglycerol); MAG (monoacylglycerols); PG (Phosphatidylglycerols); and TAG (triacylglycerols).

Experimental Details:

Lipids Extraction: Approximately 30 mg of lyophilized biomass was weighed into a glass test tube (16 mL). 100 mL of a 5000 ppm internal standard (IS) solution (perfluoroheptanoic acid—C₇HF₁₃O₂ in MeOH) was added into the test tube. 9.9 ml of extraction solvent was then added into the tube to suspend the biomass. The tube was then capped and sonicated at 50% power for 20 min, with an 80% duty cycle (20 sec on/5 s off). The extracted tubes were centrifuged at 4000 rpm/4° C. for 15 min. The supernatant was removed and transferred to an appropriate amber vial for LC/MS/MS analysis. The extraction solvent consisted of acetonitrile (35%), methanol (26%), tetrahydrofuran (9%) and methyl-tert-butyl ether (30%). The lipid yields were determined gravimetrically after evaporation of solution aliquots to dryness under a stream of nitrogen.

HPLC: A Gemini NX column (C18, 3 mm, 2.0×150 mm, s/n: 540676-12) was used for the analysis. The solvent system included: A. 85/15 MTBE/MeOH (1% 1 M NH₄Ac, 0.1% HCOOH), and B. 90/10 MeOH/Water (1% 1 M NH₄Ac, 0.1% HCOOH). The starting conditions were 5% A/95% B. After 1 minute, the gradient started and dropped to 65% B at 3 min, then 15% B at 15 minutes. It was then programmed to drop back to starting conditions (5% A/95% B) in 0.1 min, and held for 2.9 min to ensure re-equilibration. The total run time was 18 min. The flow rate was 0.3 mL/min. The column temperature was 30° C. 10 mL was injected into the system.

MS/MS: The Agilent Technologies ESI-L/Low Concentration tuning mix (Part #G1969-85000) was used to calibrate the MaXis Bruker qTOF mass spectrometer covering the range m/z 50 to 2000. The mass of the C₂₄H₁₉F₃₆N₃O₆P₃ ion structure was used as a lock mass. The instrument was tuned to a resolution of approximately 30,000.

### Example 13

### Growth Trait Genes

The complete set (131) of SN transgenic lines were also screened for growth related phenotypes. As these genes are likely involved in the nitrogen utilization pathways, the strains were screened as pools in limiting nitrogen and selected for higher levels of growth in competitive turbidostats. A turbidostat is a continuous culture device that has feedback between the turbidity of the culture vessel and the dilution rate (for example, as described in Bryson, V., & Szybalski, W. (1952). Microbial Selection. Science (New York, N.Y.), 116(3003), 45-51, doi:10.1126/science. 116.3003.45). As the turbidity increases, the media feed rate is increased to dilute the turbidity back to its set point. When the turbidity falls, the feed rate is lowered so that growth can restore the turbidity to its set point. This allows the culture to be held in an exponentially growing state for long periods, facilitating identification of specific algae lines within a population that have increased growth or a higher growth rate.

The turbidostat competition assay consists of a normalized 8×12 pool of SN genes. Each 8×12 pool represents a normalized population of 12 transgenic lines of 8 individual SN genes. Starter blocks were inoculated in 96 deep-well blocks, grown to mid to late log phase, and pooled by gene (normalized to OD). The 8 pools of transgenic strains were then combined in equal amounts in HSM media with a final concentration of 1.5 mM NH₄Cl. Growth competition assays were performed in biological triplicate in standard growth turbidostats. A baseline sample was taken at the time of turbidostat setup for sorting and calculation of the gene distribution for the starting population. The turbidostats were maintained for 2 weeks, ending with each turbidostat being sorted and screened by PCR and sequencing for final gene composition of the population. Lines that possess a competitive advantage over the other transgenic lines in the pool will increase their representation in the turbidostat relative to the starting distribution.

The Existing Genes that impact growth in C. reinhardtii are listed in Table 12 along with the Joint Genome Institute (JGI) protein ID and functional annotation. Also included below are the sequence identifier numbers for the genes.

TABLE 12

| | JGI | |
|---|---|---|
| SN | Protein ID | Functional Annotation |
| SN01 (SEQ | 179214 | Translation initiation factor 4F, |
| ID NO: 175) | | ribosome/mRNA-bridging subunit (eIF-4G) |
| SN06 (SEQ | 295492 | hypothetical protein |
| ID NO: 181) | | |
| SN24 (SEQ | 285589 | hypothetical protein |
| ID NO: 187) | | |
| SN25 (SEQ | 393275 | hypothetical protein |
| ID NO: 193) | | |
| SN28 (SEQ | 291009 | Nuclear receptor coregulator SMRT/SMRTER, |
| ID NO: 199) | | contains Myb-like domains |
| SN42 (SEQ | 297943 | hypothetical protein |
| ID NO: 205) | | |
| SN46 (SEQ | 293583 | Nuclear receptor coregulator SMRT/SMRTER, |
| ID NO: 211) | | contains Myb-like domains |
| SN47 (SEQ | 324824 | Transcription regulator dachshund, contains |
| ID NO: 217) | | SKI/SNO domain |
| SN55 (SEQ | 338285 | Acetylglucosaminyltransferase |
| ID NO: 223) | | EXT1/exostosin 1 |
| SN57 (SEQ | 121702 | Molecular chaperone (DnaJ superfamily) |
| ID NO: 229) | | |
| SN59 (SEQ | 143030 | Conserved Zn-finger protein |
| ID NO: 235) | | |
| SN64 (SEQ | 289771 | CREB binding protein/P300 and related |
| ID NO: 241) | | TAZ Zn-finger proteins |
| SN69 (SEQ | 158745 | Ammonia permease |
| ID NO: 247) | | |
| SN76 (SEQ | 192085 | Sulfite reductase (ferredoxin) |
| ID NO: 253) | | |
| SN78 (SEQ | 295739 | SWI/SNF-related matrix-associated actin- |
| ID NO: 259) | | dependent regulator of chromatin |
| SN79 (SEQ | 423635 | Nuclear inhibitor of phosphatase-1 |
| ID NO: 265) | | |
| SN82 (SEQ | 337172 | Rho GTPase effector BNI1 and related |
| ID NO: 271) | | formins |
| SN111 (SEQ | 179132 | hypothetical protein |
| ID NO: 277) | | |
| SN118 (SEQ | 311910 | hypothetical protein |
| ID NO: 283) | | |
| SN122 (SEQ | 413890 | hypothetical protein |
| ID NO: 289) | | |
| SN128 (SEQ | 417505 | hypothetical protein |
| ID NO: 295) | | |

A list of the codon-optimized gene sequences (represented by SEQ ID NOs.) that were each cloned into a Ble2A expression construct is provided below in Table 13.

SN01 (SEQ ID NO: 177)
SN06 (SEQ ID NO: 183)
SN24 (SEQ ID NO: 189)
SN25 (SEQ ID NO: 195)
SN28 (SEQ ID NO: 201)
SN42 (SEQ ID NO: 207)
SN46 (SEQ ID NO: 213)
SN47 (SEQ ID NO: 219)
SN55 (SEQ ID NO: 225)
SN57 (SEQ ID NO: 231)
SN59 (SEQ ID NO: 237)
SN64 (SEQ ID NO: 243)
SN69 (SEQ ID NO: 249)
SN76 (SEQ ID NO: 255)
SN78 (SEQ ID NO: 261)
SN79 (SEQ ID NO: 267)
SN82 (SEQ ID NO: 273)
SN111 (SEQ ID NO: 279)
SN118 (SEQ ID NO: 285)
SN122 (SEQ ID NO: 291)
SN128 (SEQ ID NO: 297)

The growth screening data is presented below in Table 14. The data below shows the frequency for each specific transgene in a population of transgenic algae strains. Baseline represents the starting population, with a target of equal representation (12.5%) of each of the 8 genes in a mix (based on OD of the starting cultures). Triplicate turbidostats (A, B, C) were run and the frequency of each transgene after two weeks in the turbidostats is shown. Those genes that increase in frequency are selected as “growth winners.”

TABLE 14

| | | | Turb A - | | Turb B - | | Turb C - | |
|---|---|---|---|---|---|---|---|---|
| | Baseline | | 2 week | | 2 week | | 2 week | |
| #1 | | | | | | | | |
| SN01 | 15 | 7.46% | 42 | 29.58% | 16 | 14.55% | 9 | 7.96% |
| SN26 | 18 | 8.96% | 6 | 4.23% | 2 | 1.82% | 2 | 1.77% |
| SN37 | 21 | 10.45% | 8 | 5.63% | 13 | 11.82% | 2 | 1.77% |
| SN43 | 36 | 17.91% | 17 | 11.97% | 20 | 18.18% | 20 | 17.70% |
| SN46 | 23 | 11.44% | 15 | 10.56% | 25 | 22.73% | 31 | 27.43% |
| SN48 | 46 | 22.89% | 9 | 6.34% | 1 | 0.91% | 15 | 13.27% |
| SN57 | 25 | 12.44% | 34 | 23.94% | 33 | 30.00% | 33 | 29.20% |
| SN68 | 17 | 8.46% | 11 | 7.75% | 0 | 0.00% | 1 | 0.88% |
| Totals | 201 | | 142 | | 110 | | 113 | |
| #2 | | | | | | | | |
| SN02 | 20 | 18.87% | 7 | 12.07% | 5 | 7.94% | 4 | 4.88% |
| SN21 | 2 | 1.89% | 4 | 6.90% | 5 | 7.94% | 3 | 3.66% |
| SN28 | 19 | 17.92% | 27 | 46.55% | 36 | 57.14% | 44 | 53.66% |
| SN30 | 3 | 2.83% | 2 | 3.45% | 1 | 1.59% | 2 | 2.44% |
| SN58 | 20 | 18.87% | 9 | 15.52% | 8 | 12.70% | 14 | 17.07% |
| SN60 | 0 | 0.00% | 4 | 6.90% | 2 | 3.17% | 2 | 2.44% |
| SN63 | 25 | 23.58% | 1 | 1.72% | 3 | 4.76% | 9 | 10.98% |
| SN70 | 17 | 16.04% | 4 | 6.90% | 3 | 4.76% | 4 | 4.88% |
| Totals | 106 | | 58 | | 63 | | 82 | |
| | | | Turb A - | | Turb B - | | Turb C - | Turb A - |
| #3 | Baseline | | 2 week | | 2 week | | 2 week | 2 week |
| SN05 | — | 0.00% | 3 | 7.50% | 1 | 1.79% | 3 | 7.14% |
| SN10 | — | 0.00% | 0 | 0.00% | 1 | 1.79% | 1 | 2.38% |
| SN15 | — | 0.00% | 3 | 7.50% | 3 | 5.36% | 0 | 0.00% |
| SN17 | — | 0.00% | 0 | 0.00% | 11 | 19.64% | 3 | 7.14% |
| SN18 | — | 0.00% | 0 | 0.00% | 2 | 3.57% | 8 | 19.05% |
| SN25 | — | 0.00% | 29 | 72.50% | 30 | 53.57% | 24 | 57.14% |
| SN73 | — | 0.00% | 3 | 7.50% | 6 | 10.71% | 3 | 7.14% |
| SN95 | — | 0.00% | 2 | 5.00% | 2 | 3.57% | 0 | 0.00% |
| Totals | — | | 40 | | 56 | | 42 | |

| | | | Turb A - | | Turb B - | | Turb C - | |
|---|---|---|---|---|---|---|---|---|
| | Baseline | | 2 week | | 2 week | | 2 week | |
| #4 | | | | | | | | |
| SN06 | 18 | 13.85% | 57 | 37.01% | 40 | 26.85% | 61 | 36.31% |
| SN16 | 17 | 13.08% | 10 | 6.49% | 4 | 2.68% | 10 | 5.95% |
| SN22 | 19 | 14.62% | 17 | 11.04% | 15 | 10.07% | 9 | 5.36% |
| SN36 | 19 | 14.62% | 6 | 3.90% | 4 | 2.68% | 11 | 6.55% |
| SN40 | 24 | 18.46% | 13 | 8.44% | 20 | 13.42% | 11 | 6.55% |
| SN45 | 6 | 4.62% | 14 | 9.09% | 10 | 6.71% | 13 | 7.74% |
| SN65 | 9 | 6.92% | 21 | 13.64% | 27 | 18.12% | 27 | 16.07% |
| SN88 | 18 | 13.85% | 16 | 10.39% | 29 | 19.46% | 26 | 15.48% |
| Totals | 130 | | 154 | | 149 | | 168 | |
| #5 | | | | | | | | |
| SN12 | 10 | 10.31% | 1 | 0.72% | 0 | 0.00% | 0 | 0.00% |
| SN14 | 10 | 10.31% | 0 | 0.00% | 0 | 0.00% | 0 | 0.00% |
| SN19 | 5 | 5.15% | 0 | 0.00% | 0 | 0.00% | 0 | 0.00% |
| SN41 | 4 | 4.12% | 3 | 2.17% | 11 | 7.24% | 8 | 6.02% |
| SN47 | 10 | 10.31% | 13 | 9.42% | 9 | 5.92% | 20 | 15.04% |
| SN76 | 14 | 14.43% | 43 | 31.16% | 85 | 55.92% | 58 | 43.61% |
| SN27 | 22 | 22.68% | 13 | 9.42% | 7 | 4.61% | 11 | 8.27% |
| SN42 | 22 | 22.68% | 65 | 47.10% | 40 | 26.32% | 36 | 27.07% |
| Totals | 97 | | 138 | | 152 | | 133 | |
| #6 | | | | | | | | |
| SN13 | 18 | 11.61% | 10 | 7.75% | 10 | 6.25% | 7 | 4.73% |
| SN23 | 17 | 10.97% | 14 | 10.85% | 7 | 4.38% | 7 | 4.73% |
| SN24 | 26 | 16.77% | 38 | 29.46% | 34 | 21.25% | 68 | 45.95% |
| SN32 | 16 | 10.32% | 12 | 9.30% | 24 | 15.00% | 11 | 7.43% |
| SN49 | 1 | 0.65% | 10 | 7.75% | 21 | 13.13% | 13 | 8.78% |
| SN66 | 32 | 20.65% | 20 | 15.50% | 25 | 15.63% | 24 | 16.22% |
| SN72 | 27 | 17.42% | 10 | 7.75% | 28 | 17.50% | 9 | 6.08% |
| SN77 | 18 | 11.61% | 15 | 11.63% | 11 | 6.88% | 9 | 6.08% |
| Totals | 155 | | 129 | | 160 | | 148 | |
| #7 | | | | | | | | |
| SN 35 | 0 | 0.00% | 3 | 1.90% | 7 | 6.09% | 0 | 0.00% |
| SN 39 | 0 | 0.00% | 1 | 0.63% | 2 | 1.74% | 1 | 0.94% |
| SN 47 | 0 | 0.00% | 1 | 0.63% | 33 | 28.70% | 58 | 54.72% |
| SN 59 | 0 | 0.00% | 119 | 75.32% | 31 | 26.96% | 11 | 10.38% |
| SN 80 | 0 | 0.00% | 4 | 2.53% | 21 | 18.26% | 3 | 2.83% |
| SN 81 | 0 | 0.00% | 18 | 11.39% | 10 | 8.70% | 1 | 0.94% |
| SN 94 | 0 | 0.00% | 6 | 3.8% | 3 | 2.61% | 28 | 26.42% |
| SN 97 | 0 | 0.00% | 6 | 3.8% | 8 | 6.96% | 4 | 3.77% |
| Totals | 0 | | 158 | | 115 | | 106 | |
| #8 | | | | | | | | |
| SN61 | 2 | 1.29% | 2 | 2.35% | 1 | 0.85% | 0 | 0.00% |
| SN71 | 17 | 10.97% | 9 | 10.59% | 4 | 3.42% | 11 | 16.92% |
| SN75 | 23 | 14.84% | 9 | 10.59% | 11 | 9.40% | 15 | 23.08% |
| SN79 | 39 | 25.16% | 46 | 54.12% | 67 | 57.26% | 12 | 18.46% |
| SN86 | 9 | 5.81% | 6 | 7.06% | 8 | 6.84% | 8 | 12.31% |
| SN93 | 30 | 19.35% | 6 | 7.06% | 18 | 15.38% | 12 | 18.46% |
| SN99 | 12 | 7.74% | 5 | 5.88% | 3 | 2.56% | 2 | 3.08% |
| SN101 | 23 | 14.84% | 2 | 2.35% | 5 | 4.27% | 5 | 7.69% |
| Totals | 155 | | 85 | | 117 | | 65 | |
| #9 | | | | | | | | |
| SN08 | 14 | 6.39% | 11 | 22.45% | 2 | 1.92% | 17 | 15.18% |
| SN09 | 24 | 10.96% | 2 | 4.08% | 7 | 6.73% | 17 | 15.18% |
| SN38 | 4 | 1.83% | 1 | 2.04% | 2 | 1.92% | 0 | 0.00% |
| SN64 | 17 | 7.76% | 7 | 14.29% | 44 | 42.31% | 4 | 3.57% |
| SN69 | 23 | 10.50% | 5 | 10.20% | 18 | 17.31% | 31 | 27.68% |
| SN87 | 20 | 9.13% | 5 | 10.20% | 12 | 11.54% | 10 | 8.93% |
| SN88 | 47 | 21.46% | 13 | 26.53% | 9 | 8.65% | 21 | 18.75% |
| SN91 | 70 | 31.96% | 5 | 10.20% | 10 | 9.62% | 12 | 10.71% |
| Totals | 219 | | 49 | | 104 | | 112 | |
| #10 | | | | | | | | |
| SN 07 | 20 | 13.70% | 10 | 7.04% | 4 | 2.82% | 8 | 5.76% |
| SN11 | 20 | 13.70% | 5 | 3.52% | 5 | 3.52% | 2 | 1.44% |
| SN34 | 8 | 5.48% | 0 | 0.00% | 2 | 1.41% | 0 | 0.00% |
| SN62 | 29 | 19.86% | 3 | 2.11% | 5 | 3.52% | 4 | 2.88% |
| SN67 | 13 | 8.90% | 1 | 0.70% | 3 | 2.11% | 2 | 1.44% |
| SN82 | 28 | 19.18% | 80 | 56.34% | 78 | 54.93% | 63 | 45.32% |
| SN84 | 15 | 10.27% | 26 | 18.31% | 28 | 19.72% | 33 | 23.74% |
| SN85 | 13 | 8.90% | 17 | 11.97% | 17 | 11.97% | 27 | 19.42% |
| Totals | 146 | | 142 | | 142 | | 139 | |
| #11 | | | | | | | | |
| SN 108 | 23 | 10.00% | 27 | 16.36% | 11 | 7.01% | 12 | 10.81% |
| SN 110 | 15 | 6.52% | 17 | 10.30% | 36 | 22.93% | 23 | 20.72% |
| SN 112 | 44 | 19.13% | 10 | 6.06% | 5 | 3.18% | 10 | 9.01% |
| SN 115 | 27 | 11.74% | 4 | 2.42% | 5 | 3.18% | 1 | 0.90% |
| SN 117 | 40 | 17.39% | 13 | 7.88% | 16 | 10.19% | 11 | 9.91% |
| SN 118 | 28 | 12.17% | 25 | 15.15% | 40 | 25.48% | 32 | 28.83% |
| SN 120 | 33 | 14.35% | 0 | 0.00% | 3 | 1.91% | 1 | 0.90% |
| SN 128 | 20 | 8.70% | 69 | 41.82% | 41 | 26.11% | 21 | 18.92% |
| Totals | 230 | 1 | 165 | 1 | 157 | 1 | 111 | 1 |
| #12 | | | | | | | | |
| SN 109 | 22 | 13.33% | 29 | 20.14% | 19 | 13.57% | 42 | 28.77% |
| SN 113 | 16 | 9.70% | 26 | 18.06% | 9 | 6.43% | 19 | 13.01% |
| SN 116 | 28 | 16.97% | 9 | 6.25% | 13 | 9.29% | 2 | 1.37% |
| SN 121 | 13 | 7.88% | 26 | 18.06% | 8 | 5.71% | 18 | 12.33% |
| SN 123 | 21 | 12.73% | 11 | 7.64% | 6 | 4.29% | 33 | 22.60% |
| SN 130 | 20 | 12.12% | 9 | 6.25% | 18 | 12.86% | 5 | 3.42% |
| SN 136 | 12 | 7.27% | 22 | 15.28% | 6 | 4.29% | 15 | 10.27% |
| SN 124 | 33 | 20.00% | 12 | 8.33% | 61 | 43.57% | 12 | 8.22% |
| Totals | 165 | 1 | 144 | 1 | 140 | 1 | 146 | 1 |
| #13 | | | | | | | | |
| SN 122 | 61 | 29.90% | 141 | 78.77% | 167 | 98.82% | 72 | 69.23% |
| SN 131 | 34 | 16.67% | 5 | 2.79% | 0 | 0.00% | 7 | 6.73% |
| SN 137 | 27 | 13.24% | 5 | 2.79% | 0 | 0.00% | 3 | 2.88% |
| SN 132 | 34 | 16.67% | 8 | 4.47% | 0 | 0.00% | 1 | 0.96% |
| SN 135 | 27 | 13.24% | 5 | 2.79% | 1 | 0.59% | 13 | 12.50% |
| SN 119 | 6 | 2.94% | 4 | 2.23% | 0 | 0.00% | 2 | 1.92% |
| SN 125 | 15 | 7.35% | 11 | 6.15% | 1 | 0.59% | 6 | 5.77% |
| SN 126 | 0 | 0.00% | 0 | 0.00% | 0 | 0.00% | 0 | 0.00% |
| Totals | 204 | 1 | 179 | 1 | 169 | 1 | 104 | 1 |
| #14 | | | | | | | | |
| SN 55 | 35 | 32.41% | 54 | 62.79% | 40 | 62.50% | 77 | 89.53% |
| SN 100 | 14 | 12.96% | 3 | 3.49% | 4 | 6.25% | 0 | 0.00% |
| SN 44 | 11 | 10.19% | 0 | 0.00% | 1 | 1.56% | 0 | 0.00% |
| SN 52 | 13 | 12.04% | 9 | 10.47% | 5 | 7.81% | 4 | 4.65% |
| SN 89 | 15 | 13.89% | 14 | 16.28% | 6 | 9.38% | 0 | 0.00% |
| SN 04 | 6 | 5.56% | 3 | 3.49% | 3 | 4.69% | 0 | 0.00% |
| SN 29 | 14 | 12.96% | 3 | 3.49% | 5 | 7.81% | 5 | 5.81% |
| SN 83 | 0 | 0.00% | 0 | 0.00% | 0 | 0.00% | 0 | 0.00% |
| Totals | 108 | 1 | 86 | 1 | 64 | 1 | 86 | 1 |
| #15 | | | | | | | | |
| SN 111 | 3 | 1.94% | 18 | 33.33% | 0 | 0.00% | 1 | 1.85% |
| SN 134 | 45 | 29.03% | 3 | 5.56% | 0 | 0.00% | 10 | 18.52% |
| SN 33 | 16 | 10.32% | 7 | 12.96% | 0 | 0.00% | 0 | 0.00% |
| SN 54 | 33 | 21.29% | 2 | 3.70% | 0 | 0.00% | 1 | 1.85% |
| SN 56 | 1 | 0.65% | 0 | 0.00% | 0 | 0.00% | 0 | 0.00% |
| SN 96 | 18 | 11.61% | 0 | 0.00% | 0 | 0.00% | 0 | 0.00% |
| SN 78 | 39 | 25.16% | 24 | 44.44% | 2 | 100.00% | 42 | 77.78% |
| SN 92 | 0 | 0.00% | 0 | 0.00% | 0 | 0.00% | 0 | 0.00% |
| SN 20 | 0 | 0.00% | 0 | 0.00% | 0 | 0.00% | 0 | 0.00% |
| Totals | 155 | 1 | 54 | 1 | 2 | 1 | 54 | 1 |

Genes nominated as “wroth winners” from each Gene Mix are presented below in Table 15.

| Gene mix no. | winners |
|---|---|
| 1 | SN01, SN46, SN57 |
| 2 | SN28 |
| 3 | SN25 |
| 4 | SN06 |
| 5 | SN42, SN76 |
| 6 | SN24 |
| 7 | SN47, SN59 |
| 8 | SN79 |
| 9 | SN64, SN69 |
| 10 | SN82 |
| 11 | SN118, SN128 |
| 12 | none |
| 13 | SN122 |
| 14 | SN55 |
| 15 | SN78, SN111 |

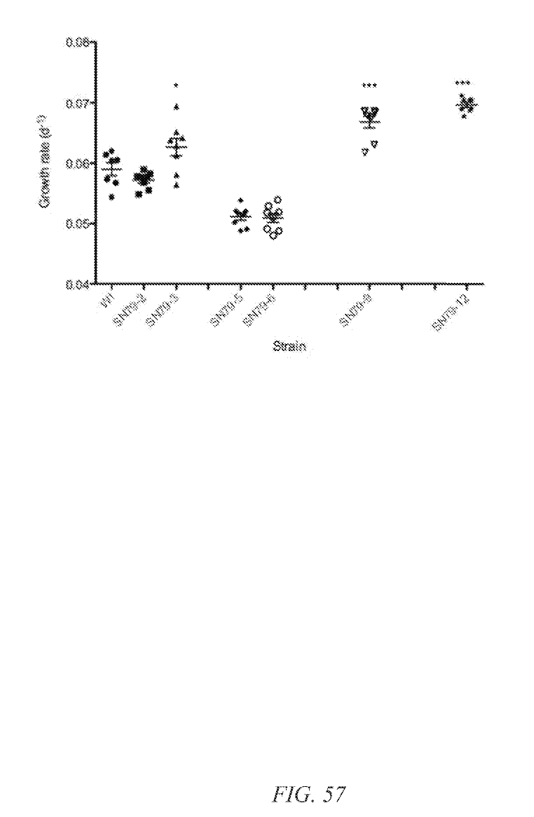

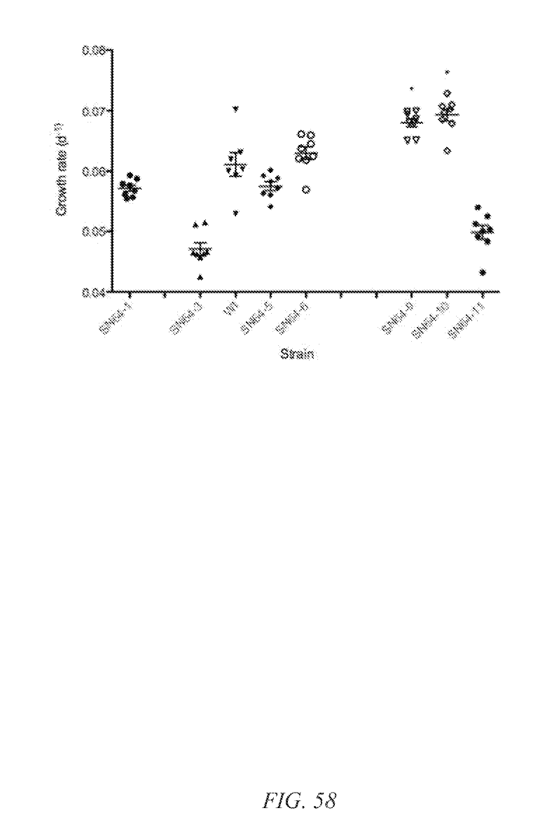

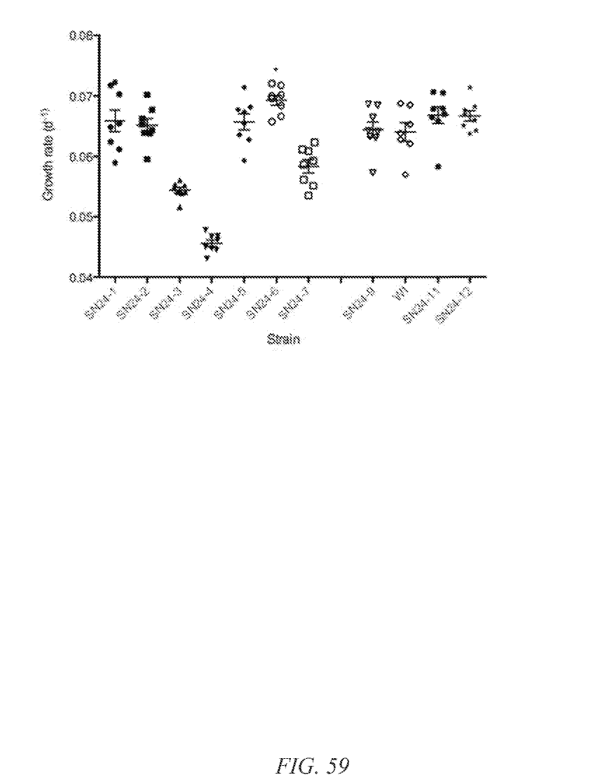

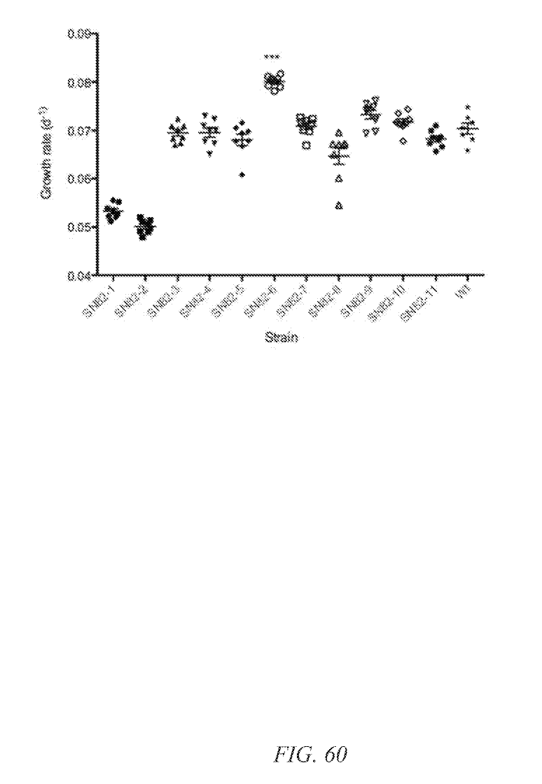

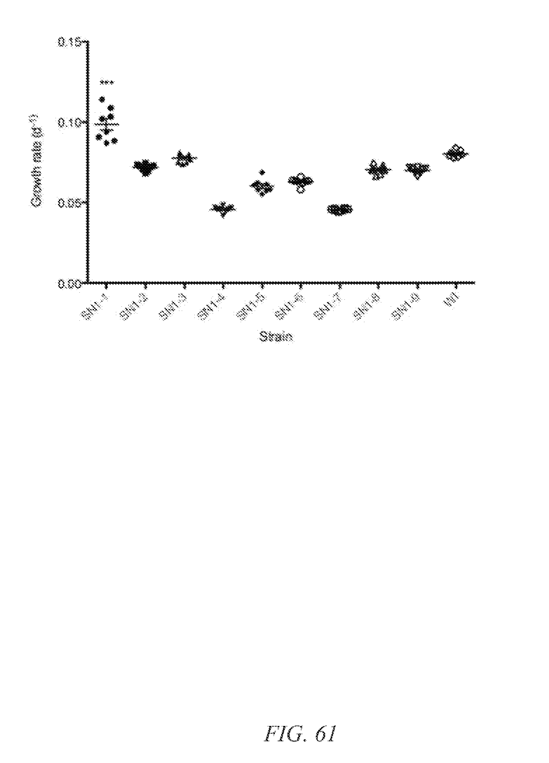

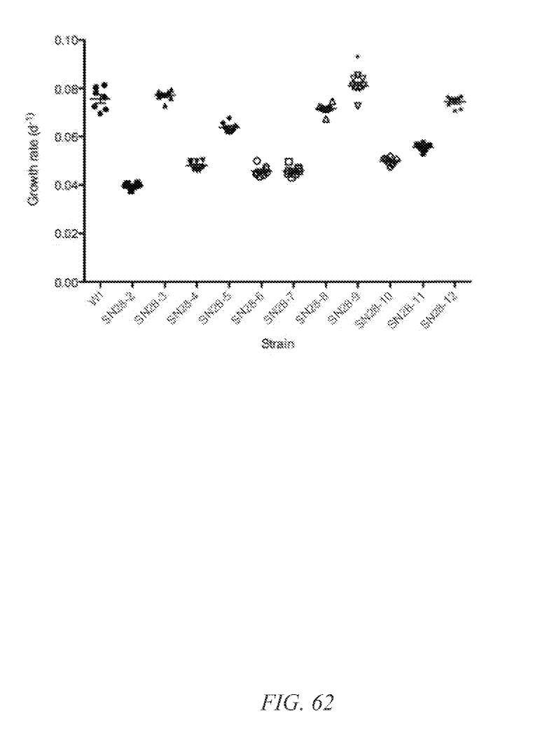

In addition to the competition growth assays described above, growth rates on up to 12 independent transgenic lines for six of the genes (SN79, 64, 24, 82, 1, and 28) were determined in growth assays. Cells were grown in a 96 well plate to full saturation. Cells were then diluted into HSM media and grown overnight. From this culture, replicates of each line were diluted into HSM media in microtiter plates at OD₇₅₀=0.02. Plates were grown under light in a 5% CO₂ environment and OD750 readings were taken every 8-16 hours. Data is plotted based on the natural log of the OD. Growth rate is taken from the slope of the curve over a period of time. Growth rates for SN79, 64, 24, 82, 1, and 28) transgenic lines along with a wild type control are shown in FIG. 57-62.

### Example 14

### Identification of Homologous Protein(s) in Other Strains of Algae

As nitrogen starvation induces lipid increases and growth changes in many species of algae, it can be expected that the SN proteins may have a conserved mechanism for inducing these changes, and therefore identifying homologous proteins in other algae strains is desirable. Bioinformatics tools such as BLAST can be used to query the published genome and transcriptome sequences of algae and other organisms. The published functional annotations of algae and other organisms for annotations similar to those of any SN gene can be searched. Candidate sequences can be aligned using ClustalW to determine identity and similarity to any SN gene. These sequences can then be expressed in any algal strain and, where applicable, in the species from which they are derived, to determine their effect on lipid accumulation and/or growth.

### Example 15

### Transcriptomics Using Additional Algae Species Under Nitrogen Starved Conditions

The approaches described in EXAMPLE 3 for SE0050 (Chlamydomonas reinhardtii) can be applied to the algae Scenedesmus dimorphus (SE0004). A reference transcriptome was generated by sequencing a normalized cDNA library using 454 technology. The library was generated from 10 different algae cultures all grown under varying treatments in order to maximize representation of all transcripts in the organism. RNA was sequenced using Solexa technology from a set of SE0004 samples grown under five nitrogen starvation and replete conditions (1:nitrogen replete, exponential growth; 2:nitrogen replete; stationary growth; 3: nitrogen starvation, 6 H; 4: nitrogen starvation, 24 H: 5: nitrogen starvation, 48 H). This RNA-Seq data has been mapped against the SE0004 reference transcriptome and genes are being identified that are involved in the nitrogen starvation pathways, including the lipid increase pathway. These genes will be over expressed and/or knocked down in SE0050 and SE0004 to determine their effect on lipid accumulation.

Table 7 shows the details of the SE0004 reference transcriptome. Under the heading RAW is listed the number of 454 sequencing reads, their average length and the total amount of sequence generated. Under the Assembled heading is listed the number of sequence contigs, their average length and the total nucleotide bases represented by the assembled reference transcriptome.

TABLE 7

| | RAW | | | Assembled | | |
|---|---|---|---|---|---|---|
| | # | average | total | # | average | total |
| | reads | length | bases | contigs | length | bases |
| SE0004 | 1,295,297 | 330 | 427.6 | 17,672 | 753 | 13.3 |
| Reference | | base | mega | | base | mega |
| | | pairs | bases | | pairs | bases |

### Example 16

### Expression of a Set of Nitrogen Starvation Induced Genes in Other Algae Species

Genes from SE0004 have been identified that show an upregulated expression pattern under nitrogen starvation, as identified by RNA-Seq transcriptomics. These genes are being cloned into expression vectors specific for SE0004, which will then be transformed into SE0004 algae. We are using SE0050 expression vectors (Ble2A, SEnuc357, and Arg7/2A) to over express in SE0050 (Chlamydomonas), genes from SE0004 identified as upregulated under nitrogen starvation. We are using SE0004 vectors to over express SN03 from SE0050 in SE0004 strains.

### Example 17

### Use of an SN DNA, RNA or Protein to Identify Interacting Molecules or Other Genes Involved in the Nitrogen Starvation Pathways

This example describes a method to use the DNA or RNA encoding an SN gene or an SN protein to identify other DNAs, RNAs or proteins and/or their corresponding genes that are involved in the nitrogen starvation pathways, whose knowledge and use can lead to manipulations of the lipid accumulation and profile in algae.

One method would be to use the SN protein expressed in vitro or from cell culture to probe high density DNA microarrays, as in (Berger et al. Compact, universal DNA microarrays to comprehensively determine transcription-factor binding site specificities. Nature Biotechnology (2006) vol. 24 (11) pp. 1429-35). This could be used to identify DNA binding sites that could then be mapped to the genome to indicate genes whose transcription is controlled by the SN protein. These genes could then be used to understand and modify the phenotypes caused by nitrogen starvation.

Another method would be to use the SN protein in a two-hybrid assay, as in (for example, as described in Miller and Stagljar. Using the yeast two-hybrid system to identify interacting proteins. Methods Mol Biol (2004) vol. 261 pp. 247-62). The SN protein can be used in this yeast system to identify other algal proteins that bind to the SN protein. The genes for these proteins could then be used to understand and modify the phenotypes caused by nitrogen starvation.

### Example 18

### Overexpression of an SN Gene in Other Organisms

Expression of Lipid or Growth Genes in Other Algal Strains.

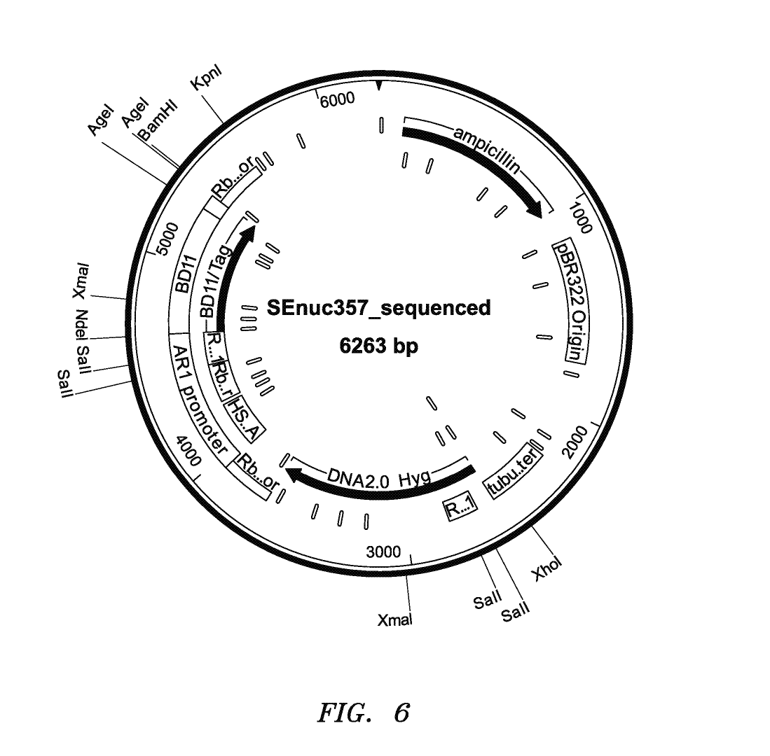

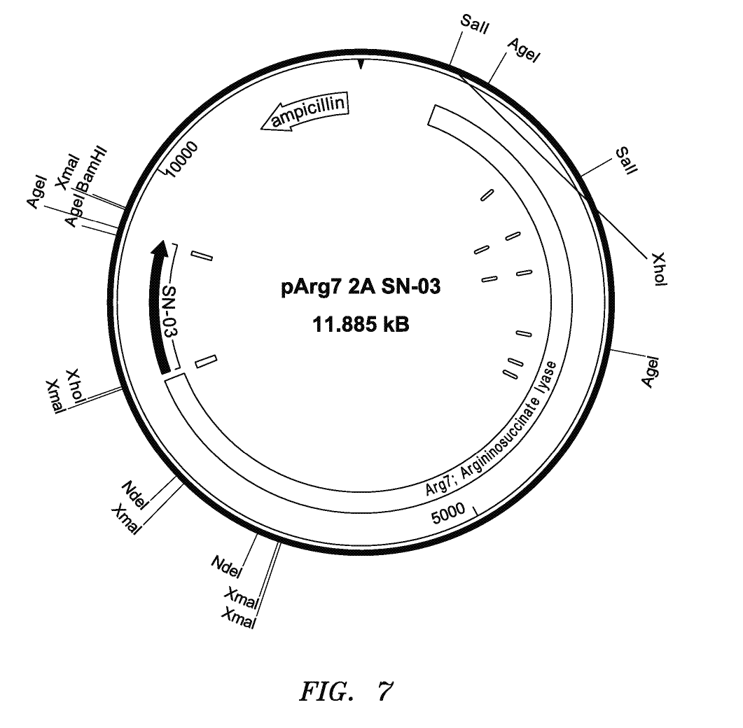

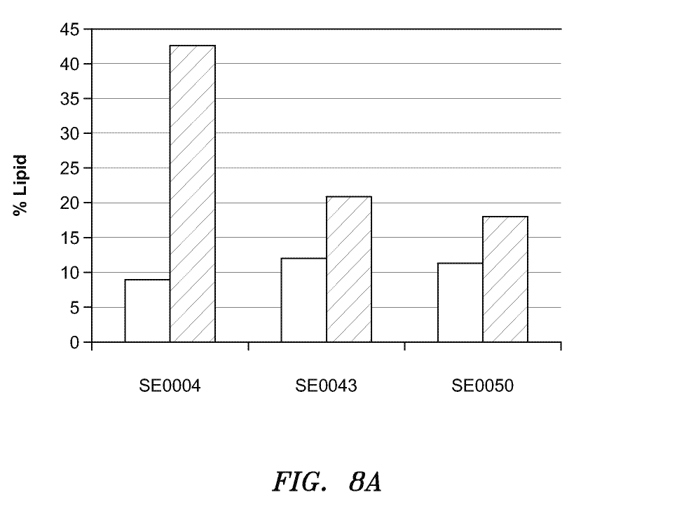

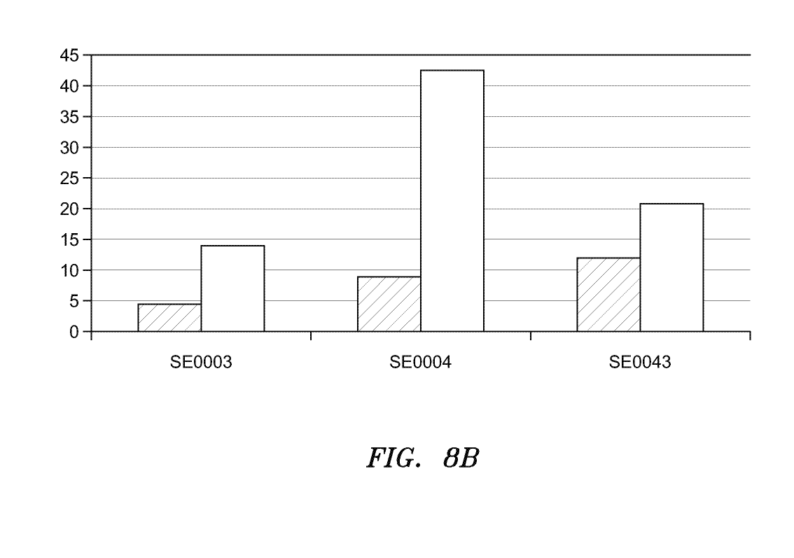

This example describes a method to overexpress an SN gene in another algae species in order to change the lipid content, lipid profile, or growth of the algal species. The SN ORF (with or without modifications and/or codon optimization) can be cloned into a transformation vector, for example, as described in FIG. 6, 7, 18, 34, 35, 63, or 64 and the protein expressed in another algal species (e.g. a Dunaliella sp., Scenedesmus sp., Desmodesmus sp., Nannochloropsis sp., Chlorella sp., Botryococcus sp., or Haematococcus sp.). Alternatively, a transformation vector with nucleotide sequence elements (for example, promoter, terminator, and/or UTR) specific to a host algae species can be used with the SN ORF. This alternate vector can also be transformed into an algae species (e.g. a Dunaliella sp. Scenedesmus sp., Desmodesmus sp., Nannochloropsis sp., Chlorella sp., Botryococcus sp., or Haematococcus sp.). Overexpression of a lipid or growth gene in any of the species described herein can be used to produce the desired phenotype.

Expression of a Lipid or Growth Gene in a Higher Plant.

This section describes a method to over express a lipid or growth gene in a higher plant, such as Arabidopsis thaliana in order to change the lipid content, lipid profile, or increase the growth of an organism.

The ORF (with or without modifications and/or codon optimization) can be cloned into a transformation vector, for example, as described in FIG. 63 or FIG. 64, a pBS SK-2×myc vector (as described in Magyar, Z. (2005) THE PLANT CELL ONLINE, 17(9), 2527-2541; doi:10.1105/tpc.105.033761), or a pMAXY4384 vector (as described in Kurek, I., et al. (2007) The Plant Cell, 19(10), 3230-3241, doi:10.1105/tpc.107.054171), and the protein expressed in, for example, a Brassica, Glycine, Gossypium, Medicago, Zea, Sorghum, Oryza, Triticum, or Panicum species.

Alternatively, a transformation vector with nucleotide sequence elements (for example, a promoter, a terminator, and/or a UTR) specific to a host plant species can be used with the lipid or growth gene ORF. This alternate vector can also be transformed into higher plant species such as Brassica, Glycine, Gossypium, Medicago, Zea, Sorghun, Oryza, Triticum, or Panicum species.

Overexpression of a lipid or growth gene in any of the species disclosed herein can be used to produce an organism with a desired phenotype (change in lipid content or lipid profile, or increased growth, for example).

### Example 19

### Combining the Effects of an SN with Other Traits or Combining Multiple SN Genes Together

This example describes multiple methods to combine SN overexpression with other transgenic lines and/or modified strains that have phenotypes different from a wild type strain.

For example, one or more additional overexpression genes could be combined with SN overexpression, either by transforming the vector containing the SN gene into a transgenic strain that already contains one or more overexpression genes, or by transforming one or more genes into a strain overexpressing the SN gene.

Another exemplary combination could be one or more knockdown or knockout genes combined with SN gene overexpression, either by transforming the vector containing the SN gene into a transgenic strain that already contains one or more knockdown or knockouts, or by transforming one or more knockout or knockdown constructs into a strain overexpressing an SN gene.

Another method would be to transform an SN gene into a strain that has been modified through mutagenesis or evolution to have a particular phenotype. Alternatively, a strain overexpressing an SN gene could be mutagenized or evolved to produce an additional phenotype.

In these approaches, the additional phenotype that is combined with the SN phenotype could be, for example, a lipid phenotype that produces additional lipid accumulation or additional lipid profile changes. Alternatively, the additional phenotype could be other than a lipid phenotype, such as a change in growth, a change in chlorophyll metabolism, resistance to some biotic or abiotic stress, or another phenotype.

One of skill in the art would be able to make numerous additional combinations, regarding the methods described above, in order to study the effects of combining the expression of an SN gene with other traits.

### Example 20

### Using SN Gene Knockdown to Identify Additional Gene(s) Involved in Nitrogen Starvation Pathway(s)

This example describes a method to identify genes involved in the nitrogen starvation phenotype using a transgenic line in which an SN gene is knocked down or knocked out. We expect that the genes whose expression is modified by knockdown of the endogenous SN gene will be a subset of the genes affected by nitrogen starvation. This data will help us understand what downstream pathways the SN protein is acting upon to produce more lipid and to alter the lipid profile.

One way to identify such genes is to grow wild type and an SN knockdown/out transgenic line in the presence and absence of nitrogen. An analysis of gene expression, protein levels and/or metabolic products could then be performed. One method to use for this analysis is the RNA-Seq methodology, which would produce lists of candidate genes based on which genes are up or down regulated in the samples.

There are many useful approaches to generating knockdown or knockouts of an SN gene. The expression of an artificial miRNA can lead to a decrease in transcript levels. Other methods of RNA silencing involve the use of a tandem inverted repeat system (Rohr et al., Plant J, 40:611-621 (2004)) where a 100-500 bp region of the targeted gene transcript is expressed as an inverted repeat. The advantage of silencing is that there can be varying degrees in which the target transcript is knocked down. Oftentimes, expression of the transcript is necessary for the viability of the cell. Thus, there can exist an intermediate level of expression that allows for both viability and also the desired phenotype (e.g. lipid induction). Finding the specific level of expression that is necessary to produce the phenotype is possible through silencing.

Homologous recombination can be carried out by a number of methods and has been demonstrated in green algae (Zorin et al., Gene, 423:91-96 (2009); Mages et al., Protist 158:435-446 (2007)). A knock out can be obtained through homologous recombination where the gene product (e.g. mRNA transcript) is eliminated by gene deletion or an insertion of exogenous DNA that disrupts the gene.

### Example 21

### Microtiter Growth Assays for SN Genes

The growth rates of multiple independent transgenic lines for several of the SN genes were determined in microtiter (microplate) growth assays. SN strains for evaluation were acclimated to a media in shaker flasks prior to starting the growth assay. Each of the SN strains were grown to mid to late log phase in 250-ml shaker flasks containing 100 ml of culture under 2-3% CO₂ and ˜65 μE/m²/s fluorescent lighting on a New Brunswick Scientific Innova 2100 Platform rotary shaker at ˜120 rpm.

After overnight growth, the cultures were transferred and normalized in the media to 3.5 ml at OD_{750 nm}=0.2 in a 24-well deep block using a Beckman Biomek fX robotic liquid handling system. Diluting back the cultures in fresh media helps maintain the nominal concentration of nutrients for the required media, since nutrient depletion may occur during media acclimation stages. The deep block was covered with a gas permeable membrane and allowed to grow under 2-3% CO₂ and ˜50 μE/m²/s fluorescent lights on a Thermo Scientific Titer Plate Shaker (model #4625) at 40% shaking speed. The shaking speed was determined by the minimal amount of speed required to maintain a suspended culture.

The following day, the cultures were normalized to 3.5 ml at OD_{750 nm}=0.02 with the media in a 24-well deep block. The normalized cultures were then randomly transferred to Costar 96-well microtiter plates (model#3903) with replication using 200 μl per well. The 96-well microtiter plates used in this assay were chosen with opaque sides to minimize position effects from light exposure across the surface and sides of the plate, and a transparent bottom to allow passage of 750 nm light during OD_{750 nm} acquisition in a 96-well microtiter plate reader. Plates were covered with a PDMS (poly dimethyl siloxane) membrane lid which allows gas exchange between the covered algae culture in each well and the chamber environment while minimizing culture volume loss to evaporation over time.

During the growth experiment, the covered plates were set into customized microtiter plate shakers in a growth chamber supplied with 5% CO₂ and incident light on the surface of the lid that can be set in the range of 50-180 μE/m²/s. Intermittent shaking was applied throughout the experiment for 15 seconds at 1700 rpm, 1 sec in each rotational direction (CW/CCW), followed by 60 seconds of no shaking. This motivation protocol is the minimal amount of agitation required to maintain sufficient suspension of the cells during the growth assay. OD_{750 nm} was acquired at ˜6 hour intervals for 96-134 hours. This is sufficient time for the cultures to reach carrying capacity at stationary phase. The resulting OD_{750 nm} data from each acquisition time point was compiled and plotted as time series.

The resulting data can be modeled in one of two ways.

The exponential growth model is based on the assumption that the rate of change of cell number is proportional to the number of cells present in the culture.

$$\frac{d N}{d t} = rN$$

which solution provides the exponential growth function,

N(t)=Nₒe^{rt}

where,
N(t)=amount of biomass at time t, measured by OD_{750 nm}
Nₒ=Initial amount of biomass, measured by OD_{750 nm}
r=specific growth rate

When modeling the data with the exponential model, only the initial data points are used as the culture only approaches unbounded exponential growth very early in the growth phase. Modeling the data in this way provides one descriptive parameter, r.

The logistic model can also be used to represent the data set. In this model, the growth rate is assumed to vary linearly with the amount of biomass, with the maximum rate being at the (relatively low) initial density and decreasing with increasing number of cells. The governing differential equation for logistic growth is

$$\frac{d N}{d t} = rN ( 1 - \frac{N}{R} )$$

The parameters are the same as previously noted, with addition of K, the carrying capacity of the system. Notice that the above equation demands that the rate of change of number of cells will approach zero as the number of cells, N, approaches the carrying capacity, K.

The solution to the above differential equation can be solved using partial fraction decomposition followed by separation of variable to obtain the logistic curve equation with the form

$$N ( t ) = \frac{K}{1 + ( \frac{K}{{N}_{0}} - 1 ) {e}^{- rt}}$$

The compiled OD_{750 nm} versus time data from each plate are imported into curve-fitting software packages and fit to the appropriate function. If the exponential fit is utilized, then the rates of the test subjects are compared. If the logistic fit is used, then an additional compound parameter is examined.

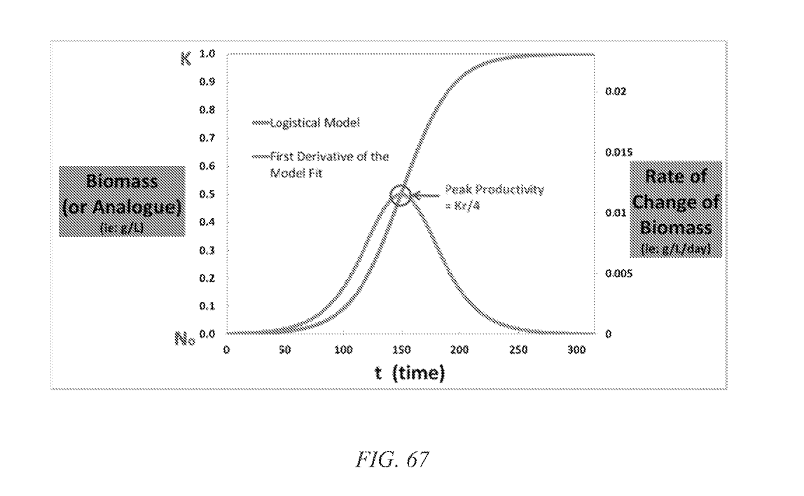

The logistic function has its maximum rate of change where the first time derivative is maximized. At this point, it can be shown that the maximum rate of change equals the compound quantity Kr/4. This ratio (Kr/4) is referred to as the peak theoretical productivity (see FIG. 67), as it represents the maximum rate of biomass accumulation for the assay conditions.

If logistic modeling is used to represent the data, all the data collected to the point at which the culture reaches stationary phase are used. Strains are compared not only by their rates (as with the exponential model), but also by their carrying capacities and peak productivities.

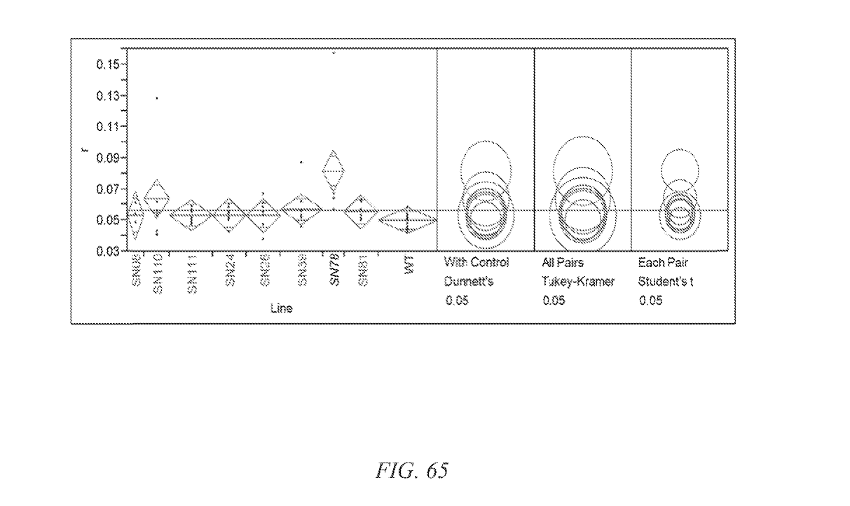

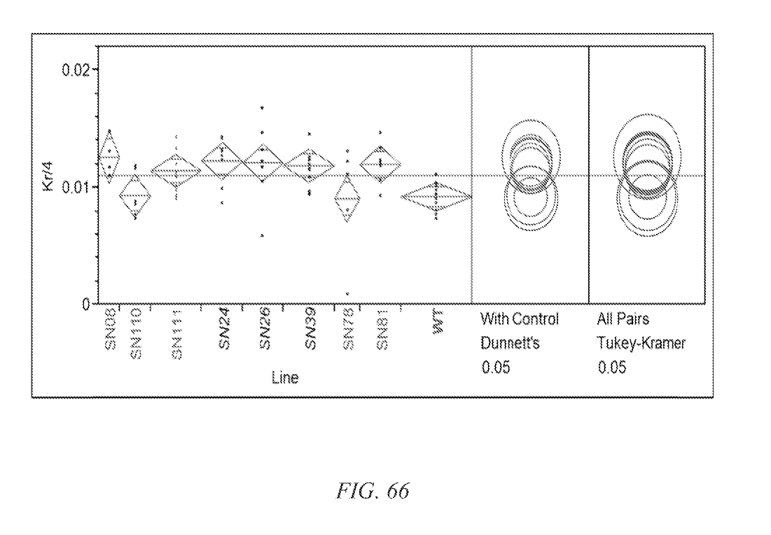

Growth rates for several of the SN transgenic lines along with a wild type control were determined and the data analyzed by Oneway ANOVA of “r” (growth rate) of individual SN gene transformants (FIG. 65), or by Oneway ANOVA of “Kr/4” of individual SN gene transformants (FIG. 66). SN78 was analyzed in FIG. 65, and SN24, SN26, and SN39 were analyzed in FIG. 66. Regarding FIG. 65, the Mean for Oneway ANOVA of SN78 was 0.081800 with a Standard Deviation of 0.00684. For SN78, the means comparison with a control (wild type) using Dunnett's Method yielded a p-Value of 0.0014. Regarding FIG. 66, the Mean for Oneway ANOVA of SN24, SN26, and SN39 was 0.012291, 0.012138, and 0.011896 respectively, with a Standard Deviation of 0.00079, 0.00079, and 0.00071 respectively. For SN24, SN26, and SN39, the means comparison with a control (wild type) using Dunnett's Method yielded a p-Value of 0.0235, 0.0358, and 0.0415 respectively.

Analysis of Variance (ANOVA) is a statistical test used to determine if more than two population means are equal. The test uses the F-distribution (probability distribution) function and information about the variances of each population (within) and grouping of populations (between) to help decide if variability between and within each population are significantly different.

Dunnett's test (method) is a statistical tool known to one skilled in the art and is described, for example, in Dunnett, C. W. (1955) “A multiple comparison procedure for comparing several treatments with a control”, Journal of the American Statistical Association, 50:1096-1121, and Dunnett, C. W. (1964) “New tables for multiple comparisons with a control”, Biometrics, 20:482-491. Dunnett's test compares group means. It is specifically designed for situations where all groups are to be pitted against one “Reference” group. It is commonly used after ANOVA has rejected the hypothesis of equality of the means of the distributions (although this is not necessary from a strictly technical standpoint). The goal of Dunnett's test is to identify groups whose means are significantly different from the mean of this reference group. It tests the null hypothesis that no group has its mean significantly different from the mean of the reference group.

### Example 22

### Lipid Analyses for SN Genes

The lipid content of multiple independent transgenic lines for several of the SN genes was determined. A lipid dye-based assay (as discussed above) was used to screen the SN transgenic lines for lipid content. Analytical flow cytometry (Guava) is a direct measurement of fluorescence that can be used when cultures are stained separately with three lipid dyes; Bodipy, Nile Red and LipidTOX Green. All three dyes are lipophilic, with specific, but ill-defined, affinities for different lipid components in a cell. Use of three different dyes provides a wider range of possible lipid phenotypes that can be observed. Of interest are SN genes that change the overall amount of lipid, but also in those that modify the lipid profile by affecting a subset of lipids. Each individual SN line was measured and compared to a wild-type C. reinhardtii line. Winners were determined based on their performance relative to the wild-type control in the Guava screen. Winners include at least one or more transformant of: SN1, SN9, SN11, SN21, SN26, SN39, SN71, SN80, SN110, SN120, and SN124.

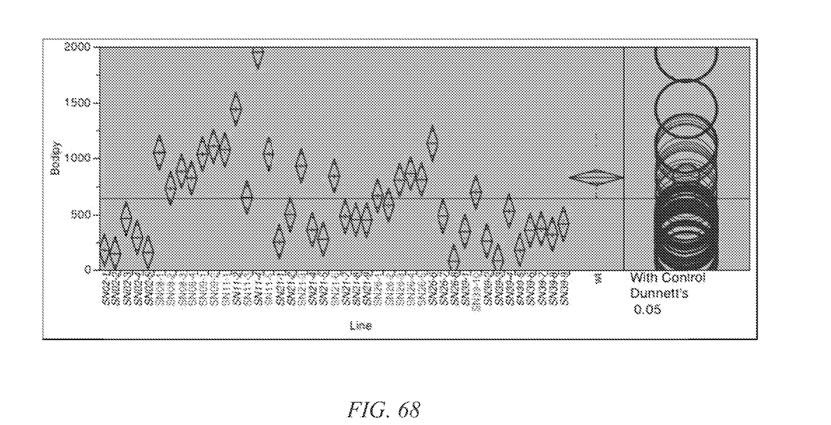

The data was analysed by Oneway ANOVA of Bodipy, Oneway ANOVA of Nile Red, and Oneway ANOVA of LipidTox staining as shown in FIG. 68 to FIG. 72. The means comparisons with a control group (wild type) using Dunnett's Method for the data presented in FIG. 68 to FIG. 72 is presented in Table 16 below.

Abs(Dif)−LSD=Absolute(Difference)−Least Significant Difference.

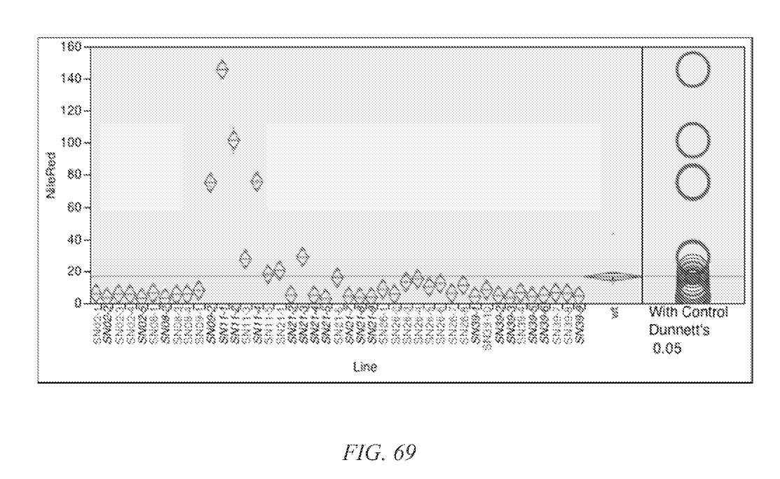

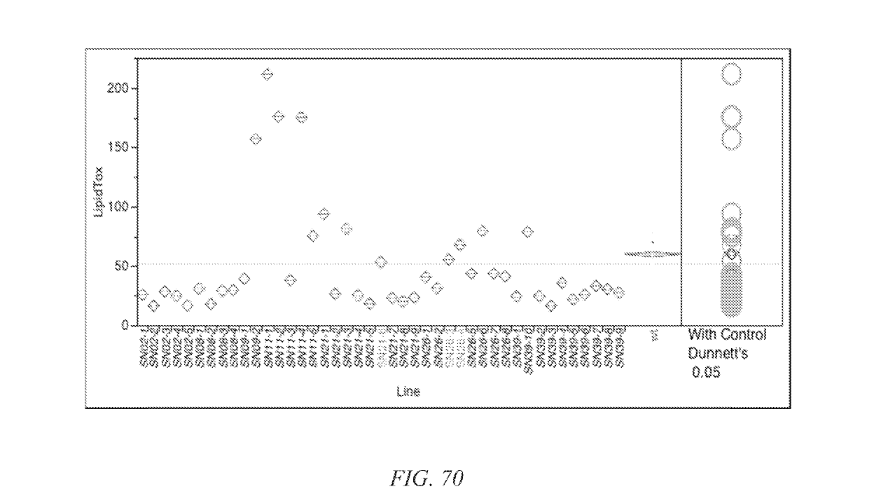

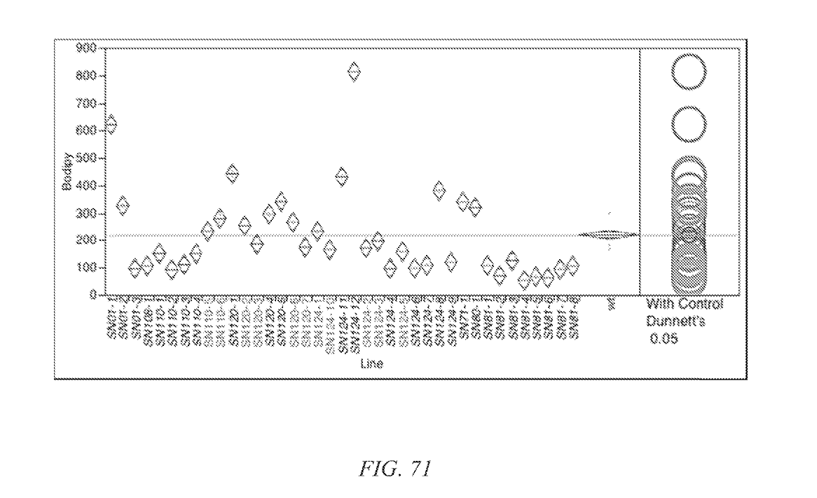

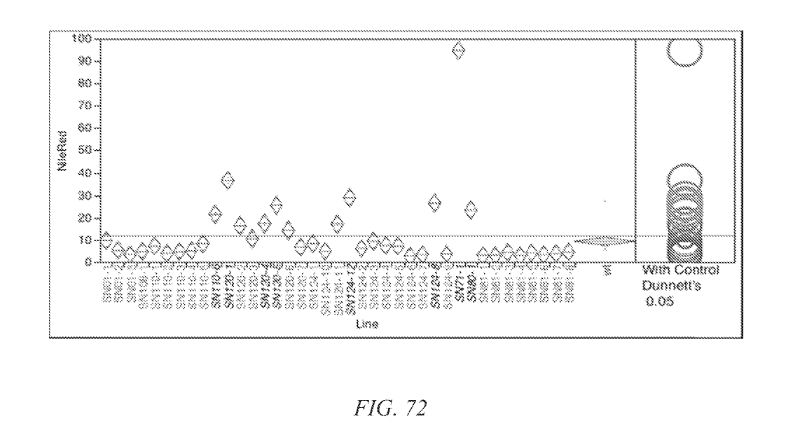

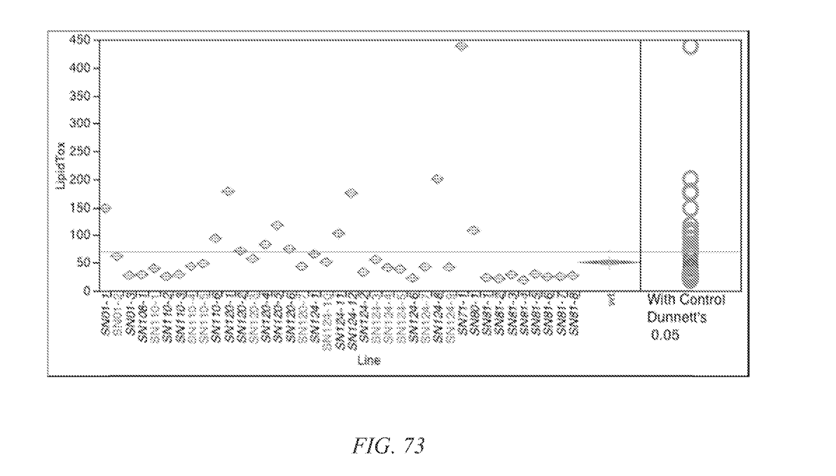

TABLE 16

| SN transgenic line | Abs(Dif) − LSD | p-Value |
|---|---|---|
| FIG. 68 | | |
| SN11-4 | 832.9 | <.0001 |
| SN11-2 | 326.6 | <.0001 |
| SN26-6 | 17.68 | 0.0275 |
| FIG. 69 | | |
| SN11-1 | 117.8 | <.0001 |
| SN11-2 | 73.71 | <.0001 |
| SN11-4 | 47.93 | <.0001 |
| SN09-2 | 47.32 | <.0001 |
| SN21-3 | 0.8 | 0.0254 |
| FIG. 70 | | |
| SN11-1 | 142 | <.0001 |
| SN11-2 | 106.2 | <.0001 |
| SN11-4 | 105.5 | <.0001 |
| SN09-2 | 87.5 | <.0001 |
| SN21-1 | 24.34 | <.0001 |
| SN21-3 | 11.81 | <.0001 |
| SN26-6 | 10.02 | <.0001 |
| SN39-10 | 8.972 | <.0001 |
| SN11-5 | 5.817 | <.0001 |
| FIG. 71 | | |
| SN124-12 | 527 | <.0001 |
| SN01-1 | 335.8 | <.0001 |
| SN120-1 | 156 | <.0001 |
| SN124-11 | 144.7 | <.0001 |
| SN124-8 | 94.92 | <.0001 |
| SN120-5 | 54.6 | <.0001 |
| SN71-1 | 53.37 | <.0001 |
| SN01-2 | 39.2 | <.0001 |
| SN80-1 | 33.36 | 0.0003 |
| SN120-4 | 8.645 | 0.0144 |
| FIG. 72 | | |
| SN71-1 | 77.55 | <.0001 |
| SN120-1 | 19.36 | <.0001 |
| SN124-12 | 11.6 | <.0001 |
| SN124-8 | 9.222 | <.0001 |
| SN120-5 | 8.277 | <.0001 |
| SN80-1 | 6.082 | <.0001 |
| SN110-6 | 4.272 | 0.0001 |
| SN120-4 | 0.152 | 0.0416 |
| FIG. 73 | | |
| SN71-1 | 372.4 | <.0001 |
| SN124-8 | 134.9 | <.0001 |
| SN120-1 | 112.7 | <.0001 |
| SN124-12 | 109.6 | <.0001 |
| SN01-1 | 82.68 | <.0001 |
| SN120-5 | 51.95 | <.0001 |
| SN80-1 | 42.98 | <.0001 |
| SN124-11 | 37.63 | <.0001 |
| SN110-6 | 29.04 | <.0001 |
| SN120-4 | 17.89 | <.0001 |
| SN120-6 | 9.737 | <.0001 |
| SN120-2 | 6.172 | 0.0006 |
| SN124-1 | 0.497 | 0.0362 |

Gene Deletion

One such way is to PCR amplify two non-contiguous regions (from several hundred DNA base pairs to several thousand DNA base pairs) of the gene. These two non-contiguous regions are referred to as Homology Region 1 and Homology Region 2 are cloned into a plasmid. The plasmid can then be used to transform the host organism to create a knockout.

Gene Insertion

Another way is to PCR amplify two contiguous or two non-contiguous regions (from several hundred DNA base pairs to several thousand DNA base pairs) of the gene. A third sequence is ligated between the first and second regions, and the resulting construct is cloned into a plasmid. The plasmid can then be used to transform the host organism to create a knockout. The third sequence can be, for example, an antibiotic selectable marker cassette, an auxotrophic marker cassette, a protein expression cassette, or multiple cassettes.

How to Measure an Increase in Growth of a Cell Line.

This section describes exemplary methods that can be used to determine an increase in the growth of a cell line.

An increase in the growth of a cell line can be measured by a competition assay, growth rate, carrying capacity, measuring culture productivity, cell proliferation, seed yield, organ growth, or polysome accumulation. These types of measurements are known to one of skill in the art.

The growth of the organism can be measured by optical density, dry weight, by total organic carbon, or by other methods known to one of skill in the art. These measurements can be, for example, fit to a growth curve to determine the maximal growth rate, the carrying capacity, and the culture productivity (for example, g/m2/day; a measurement of biomass produced per unit area/volume per unit time). These values can be compared to an untransformed cell line or another transformed cell line, to calculate the increase in growth in the overexpressing cell line of interest.

Carrying capacity can be measured, for example, as grams per liter, grams per meter cubed, grams per meter squared, or kilograms per acre. One of skill in the art would be able to choose the most appropriate units. Any mass per unit of volume or area can be measured.

Culture productivity can be measured, for example, as grams per meter squared per day, grams per liter per day, kilograms per acre per day, or grams per meter cubed per day. One of skill in the art would be able to choose the most appropriate units.

Growth rate can be measured, for example, as per hour, per day, per generation or per week. One of skill in the art would be able to choose the most appropriate units. Any per unit time can be measured.

Growth Rate

A increase in the growth rate of an organism transformed with an SN gene as compared to an untransformed or wild type organism or to another transformed organism can be, for example, about 2%, about 4%, about 6%, about 8%, about 10%, about 12%, about 14%, about 16%, about 18%, about 20%, about 22%, about 24%, about 26%, about 28%, about 30%, about 50%, about 100%, about 150%, about 200%, about 250%, about 300%, about 350%, or about 400%.

A increase in the growth rate of an organism transformed with an SN gene as compared to an untransformed or wild type organism or to another transformed organism can be, for example, at least 2%, at least 4%, at least 6%, at least 8%, at least 10%, at least 12%, at least 14%, at least 16%, at least 18%, at least 20%, at least 22%, at least 24%, at least 26%, at least 28%, at least 30%, at least 50%, at least 100%, at least 150%, at least 200%, at least 250%, at least 300%, at least 350%, or at least 400%.

While certain embodiments have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1-4. (canceled)

5. A photosynthetic organism transformed with an isolated polynucleotide comprising:
(a) a nucleic acid sequence of SEQ ID NO: 113, 65, 77, 83, 89, 95, 101, 107, 131, 119, 125, 137, 143, 149, 155, 161, 167 or 173;
(b) a nucleotide sequence with at least 80%, at least 85%, at least 90%, at least 95%, at least 98%, or at least 99% sequence identity to the nucleic acid sequence of SEQ ID NO: 113, 65, 77, 83, 89, 95, 101, 107, 131, 119, 125, 137, 143, 149, 155, 161, 167 or 173;
(c) a nucleic acid sequence of SEQ ID NO: 112, 64, 76, 82, 88, 94, 100, 106, 130, 118, 124, 136, 142, 148, 154, 160, 166, or 172; or
(d) a nucleotide sequence with at least 80%, at least 85%, at least 90%, at least 95%, at least 98%, or at least 99% sequence identity to the nucleic acid sequence of 112, 64, 76, 82, 88, 94, 100, 106, 130, 118, 124, 136, 142, 148, 154, 160, 166, or 172;
wherein the transformed organism's lipid content or profile is different than an untransformed organism's lipid content or profile.

6-10. (canceled)

11. The transformed photosynthetic organism of claim 5, wherein the transformed organism is grown in an aqueous environment.

12. The transformed photosynthetic organism of claim 5, wherein the transformed organism is a vascular plant.

13. The transformed photosynthetic organism of claim 5, wherein the transformed organism is a non-vascular photosynthetic organism.

14. The transformed photosynthetic organism of claim 5, wherein the transformed organism is an alga or a bacterium.

15. The transformed photosynthetic organism of claim 14, wherein the bacterium is a cyanobacterium.

16. The transformed photosynthetic organism of claim 15, wherein the cyanobacterium is a Synechococcus sp., Synechocystis sp., Athrospira sp., Gleocapsa sp., Spirulina sp., Leptolyngbya sp., Lyngbya sp., Oscillatoria sp., or Pseudoanabaena sp.

17. The transformed photosynthetic organism of claim 14, wherein the alga is a microalga.

18. The transformed photosynthetic organism of claim 17, wherein the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp.

19. The transformed photosynthetic organism of claim 17, wherein the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus.

20. The transformed photosynthetic organism of claim 5, wherein the transformed photosynthetic organism's nuclear genome is transformed.

21. The transformed photosynthetic organism of claim 5, wherein the transformed photosynthetic organism's chloroplast genome is transformed.

22. (canceled)

23. A method of increasing production of a lipid, comprising: i) transforming an organism with a polynucleotide comprising a nucleotide sequence encoding a protein that when expressed in the organism results in the increased production of the lipid as compared to an untransformed organism, and wherein the nucleotide sequence comprises:
(a) a nucleic acid sequence of SEQ ID NO: 113, 65, 77, 83, 89, 95, 101, 107, 131, 119, 125, 137, 143, 149, 155, 161, 167 or 173;
(b) a nucleotide sequence with at least 80%, at least 85%, at least 90%, at least 95%, at least 98%, or at least 99% sequence identity to the nucleic acid sequence of SEQ ID NO: 113, 65, 77, 83, 89, 95, 101, 107, 131, 119, 125, 137, 143, 149, 155, 161, 167 or 173;
(c) a nucleic acid sequence of SEQ ID NO: 112, 64, 76, 82, 88, 94, 100, 106, 130, 118, 124, 136, 142, 148, 154, 160, 166, or 172; or
(d) a nucleotide sequence with at least 80%, at least 85%, at least 90%, at least 95%, at least 98%, or at least 99% sequence identity to the nucleic acid sequence of 112, 64, 76, 82, 88, 94, 100, 106, 130, 118, 124, 136, 142, 148, 154, 160, 166, or 172.

24-27. (canceled)

28. The method of claim 23, wherein the transformed organism is grown in an aqueous environment.

29. The method of claim 23, wherein the transformed organism is a vascular plant.

30. The method of claim 23, wherein the transformed organism is a non-vascular photosynthetic organism.

31. The method of claim 23, wherein the transformed organism is an alga or a bacterium.

32. The method of claim 31, wherein the bacterium is a cyanobacterium.

33. The method of claim 32, wherein the cyanobacterium is a Synechococcus sp., Synechocystis sp., Athrospira sp., Gleocapsa sp., Spirulina sp., Leptolyngbya sp., Lyngbya sp., Oscillatoria sp., or Pseudoanabaena sp.

34. The method of claim 31, wherein the alga is a microalga.

35. The method of claim 34, wherein the microalga is at least one of a Chlamydomonas sp., Volvacales sp., Desmid sp., Dunaliella sp., Scenedesmus sp., Chlorella sp., Hematococcus sp., Volvox sp., Nannochloropsis sp., Arthrospira sp., Sprirulina sp., Botryococcus sp., Haematococcus sp., or Desmodesmus sp.

36. The method of claim 34, wherein the microalga is at least one of Chlamydomonas reinhardtii, N. oceanica, N. salina, Dunaliella salina, H. pluvalis, S. dimorphus, Dunaliella viridis, N. oculata, Dunaliella tertiolecta, S. Maximus, or A. Fusiformus.

37. The method of claim 23, wherein the transformed organism's nuclear genome is transformed.

38. The method of claim 23, wherein the transformed organism's chloroplast genome is transformed.

39. (canceled)

40. A higher plant transformed with an isolated polynucleotide comprising:
(a) a nucleic acid sequence of SEQ ID NO: 113, 65, 77, 83, 89, 95, 101, 107, 131, 119, 125, 137, 143, 149, 155, 161, 167 or 173;
(b) a nucleotide sequence with at least 80%, at least 85%, at least 90%, at least 95%, at least 98%, or at least 99% sequence identity to the nucleic acid sequence of SEQ ID NO: 113, 65, 77, 83, 89, 95, 101, 107, 131, 119, 125, 137, 143, 149, 155, 161, 167 or 173;
(c) a nucleic acid sequence of SEQ ID NO: 112, 64, 76, 82, 88, 94, 100, 106, 130, 118, 124, 136, 142, 148, 154, 160, 166, or 172; or
(d) a nucleotide sequence with at least 80%, at least 85%, at least 90%, at least 95%, at least 98%, or at least 99% sequence identity to the nucleic acid sequence of 112, 64, 76, 82, 88, 94, 100, 106, 130, 118, 124, 136, 142, 148, 154, 160, 166, or 172;
wherein the transformed plant's lipid content or profile is different than an untransformed plant's lipid content or profile.

41-44. (canceled)

45. The transformed higher plant of claim 40, wherein the higher plant is Arabidopsis thaliana.

46. The transformed higher plant of claim 40, wherein the higher plant is a Brassica, Glycine, Gossypium, Medicago, Zea, Sorghum, Oryza, Triticum, or Panicum species.

47-73. (canceled)
